(19) United States
(12) Patent Application Publication
(10) Pub. No.: US 2002/0151028 A1
(43) Pub. Date: Oct. 17, 2002

(54) Structure-based drug design for Ulp1 protease substrates

(75) Inventors: Christopher D. Lima, New York, NY (US); Elena Mossessova, New York, NY (US)

Correspondence Address: DARBY & DARBY P.C., 805 Third Avenue, New York, NY, 10022, US

(73) Assignee: Cornell Research Foundation, Inc.

(21) Appl. No.: 09/856,247

(22) Filed: May 17, 2001

Related U.S. Application Data
(60) Provisional application No. 60/205,336, filed on May 18, 2000.

(51) Int. Cl. C12N 9/64; G06F 19/00; G01N 33/48; G01N 33/50

(52) U.S. Cl. 435/226; 435/7.1; 702/19

(57) ABSTRACT

The present invention provides for a composition that is comprised of a polypeptide that comprises the catalytic domain of a SUMO protease and its substrate. The invention also discusses a method of trapping a protease with its substrate in a deacylation intermediate. A method of identifying substrates of proteases by rational drug design is further discussed and described. Sequences, vectors, and host cells of mutant Ulp1 are also provided.

This application claims priority under 35 U.S.C. §119 from provisional patent application Ser. No. 60/205,336, filed May 18, 2000; which is hereby incorporated by reference in its entirety.

### FIELD OF THE INVENTION

The present invention relates to rational drug design of cysteine protease inhibitors based on the crystal structure of a Ulp1 protease trapped with its substrate in a covalent intermediate.

### BACKGROUND OF THE INVENTION

Ubiquitin (Ub) and ubiquitin-like (Ubl) proteins modulate protein function in the cell through covalent modification of the target protein. Two proteins that have been identified as belonging to this family are ubiquitin and Small Ubiquitin-related Modifier (SUMO-1). The Ub/Ubl conjugated state of cellular proteins has been associated with several critical pathways, including cellular differation, apoptosis, the cell-cycle, and cellular responses to stress (Hershko and Ciehanover, 1998, Annu. Rev. Biochem., 67:425; Hochstrasser, 1998, Genes and Develop., 12:901; Laney and Hochstrasser, 1999; Cell, 97:427, Saitoh, et al., 1997, TIBS, 22:374; Johnson and Hochstrasser, 1997, Trends. Cell. Biol., 7:408). Alterations in the regulation of the Ub/Ubl pathway has been implicated in several disease states, including tumorogenesis and acute promyelocytic leukemia (Hershko and Ciehanover, 1998, Annu. Rev. Biochem., 67:425; Kamitani et al., 1998, J. Biol. Chem., 273:26675).

Ub and Ubl proteins are covalently attached to their cellular targets via a reversible amide linkage between a lysine ε-amino group on the target and the C-terminus of the Ub/Ubl protein. The fate of the modified protein depends on if it is conjugated to ubiquitin or SUMO-1. Ubiquinated and poly-ubiquinated proteins are generally targeted to the 26S proteosome for degradation in cell-cycle dependent and cell-cycle independent pathways. Sumoylated protein targets appear to modulate the activity of the targeted system either directly or indirectly by altering cellular localization. Cellular pathways that appear to be modulated by the SUMO pathway include activation of RanGAPl, p53 transcriptional regulation, and lκbα protection from ubiquitination (Matunis,et al., 1996;J. Cell. Biol., 135:1457,Gostissa, et al., 1999,EMBO J., 18:6462; Rodriguez, et al., 1999, EMBO J., 18:6455, Desterro, et al., 1998; Mol. Cell., 2:233). Ub/Ubl modifiers are generally deconjugated from their cellular targets by a class of cysteine proteases, known as deubiquinating (DUB) enzymes.

Studies in yeast show that Smt3, a yeast member of the Ubl protein family, conjugation and deconjugation to proteins appears critical to several yeast functions, including septin ring formation, chromosomal segregation, and progression of the cell cycle. Smt3 shares 47% sequence identity with mammalian SUMO-1 and 17% sequence identity with ubiquitin. Recently, studies showed the identification of a novel Saccharomyces cerevisiae gene product termed Ubl-specific protease 1 (Ulp1). In contrast to the ubiquitin DUB system, Ulp1 catalyzes Smt3 processing and Smt3-protein deconjugation.

There is a continuing need in the art to develop methods that enable one to evaluate interactions between proteases and their substrates. The development of an efficient method of trapping the protease and substrate in a complex, which would allow for the study of these interactions is needed. Such a method could allow for the development of compounds that could specifically interact with the amino acids necessary for protease function.

U.S. Pat. No. 5,834,228 discloses the use of rational drug design for developing inhibitors of the apopain that will form non-covalent interactions with active site, as based on the crystal structure of the protease and substrate. The reference, however, does not indicate how to trap the protease and substrate in a reaction intermediate state or use this method for identifying compounds that produce covalent interactions with the protease. Thus, there is a need in the art to define active site structures of cystine proteases complexed with their substrates.

### SUMMARY OF THE INVENTION

The present invention provides for a composition that is comprised of a polypeptide that comprises the catalytic domain of a SUMO protease and its substrate. In the composition, the SUMO protease is trapped in a deacylation intermediate complex with the substrate.

The invention further provides for a method of forming a complex between a polypeptide that comprises the catalytic domain of a protease with its substrate by (1) combining the protease and substrate in a molar ratio; (2) adding a reducing agent, which is capable of trapping a proteolytic deacylation intermediate complex of the protease and substrate, in an amount that is effective to trap the protease and substrate and (3) adjusting the pH of the mixture to about 7.0.

The present invention provides for polynucleotide sequence that encodes a mutant Ulp1. The mutant Ulp1 may contain an amino acid substitution at position 432, 448, 451 472, 474, 489, 490, 493, or 515. The invention also provides for vectors containing polynucleotide sequences of the mutant Ulp1, cells with the vectors containing the mutant Ulp1 polynucleotide sequences, and the mutant Ulp1 polypeptides.

The present invention also advantageously provides a method of identifying potential substrates of cystine proteases by rational drug design. The method is comprised of designing candidate substrates that form interactions with catalytic amino acids which are identified from computer modeling studies based on the crystal structure of the complex of the protease and substrate.

### BRIEF DESCRIPTION OF DRAWINGS

FIG. 1A and 1B. Stereo images of the Ulp1 active site in complex with Smt3. Hydrogen bonds are represented as black spheres. Cys580, His541, and Asp351 represent the catalytic triad in the protease fold.

### DETAILED DESCRIPTION OF THE INVENTION

The present invention advantageously provides an isolated, preferably purified, trapped protease-substrate complex, preferably a cysteine protease. In particular, protease-substrate complexes of the invention are those of the sumo pathway, involved in many important cellular processes.

The invention is based, in part, on trapping and crystallization of a Ulp1-Smt3 covalent complex, from which the Ulp1 crystal structure was obtained (coordinates are attached as Table 1 (after the specification and before the claims) and deposited in the Protein Data Bank with accession no. NC-001 148). This crystal structure permits the rational drug design of Ulp1 and Ulp1 ortholog inhibitions, which function to promote ubiquitin and ubiquitin-like protein activities in cells.

General Definitions

As used herein, the term “isolated” means that the referenced material is removed from the environment in which it is normally found. Thus, an isolated biological material can be free of cellular components, i.e., components of the cells in which the material is found or produced. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e., contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well-known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting [FACS]). Other purification methods are possible. A purified material may contain less than about 50%, preferably less than about 75%, and most preferably less than about 90%, of the cellular components with which it was originally associated. The “substantially pure” indicates the highest degree of purity which can be achieved using conventional purification techniques known in the art. In a specific embodiment, the term “about” or “approximately” means within 20%, preferably within 10%, and more preferably within 5% of a given value or range.

A “sample” as used herein refers to a biological material which can be tested for the presence of Ulp1 protein or Ulp1 nucleic acids. Such samples can be obtained from animal subjects, such as humans and non-human animals, and include tissue, biopsies, blood and blood products; plural effusions; cerebrospinal fluid (CSF); ascites fluid; and cell culture.

The term “about” or “approximately” means within an acceptable error range for a given measurement. In one aspect, the error range can be within 25% of a value, preferably within 10%, and more preferably within 5%. Alternatively, particularly in biological systems, an acceptable error is one order of magnitude, preferably 2-fold.

The use of italics indicates a nucleic acid molecule (e.g., Ulp1 cDNA, gene, etc.); normal text indicates the polypeptide or protein.

Cloning and Expression of mutant Ulp1

The present invention contemplates generation of a gene encoding wild-type or a mutant Ulp1, including a full length and any antigenic fragments thereof from any source, preferably human. It further contemplates detection of mutant Ulp1 protein for evaluation, diagnosis, or therapy.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984), F. M. Ausubel et al. (eds.), Current Protocols in Molecular Biology, John Wiley & Sons, Inc. (1994).

Molecular Biology—Definitions

“Amplification” of DNA as used herein denotes the use of polymerase chain reaction (PCR) to increase the concentration of a particular DNA sequence within a mixture of DNA sequences. For a description of PCR see Saiki et al., Science, 239:487, 1988.

The polynucleotides encoding Ulp1 herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra.

Proteins and enzymes are made in the host cell using instructions in DNA and RNA, according to the genetic code. Generally, a DNA sequence having instructions for a particular protein or enzyme is “transcribed” into a corresponding sequence of RNA. The RNA sequence in turn is “translated” into the sequence of amino acids which form the protein or enzyme. An “amino acid sequence” is any chain of two or more amino acids. Each amino acid is represented in DNA or RNA by one or more triplets of nucleotides. Each triplet forms a codon, corresponding to an amino acid. For example, the amino acid lysine (Lys) can be coded by the nucleotide triplet or codon AAA or by the codon AAG. (The genetic code has some redundancy, also called degeneracy, meaning that most amino acids have more than one corresponding codon.) Because the nucleotides in DNA and RNA sequences are read in groups of three for protein production, it is important to begin reading the sequence at the correct amino acid, so that the correct triplets are read. The way that a nucleotide sequence is grouped into codons is called the “reading frame.”

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” or “operatively associated with” of transcriptional and translational control sequences in a cell when RNA polymerase transcribes the coding sequence into mRNA, which is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i. e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence which initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is a such an element operatively associated with a different gene than the one it is operatively associated with in nature. In the context of the present invention, a Ulp1 gene is heterologous to the vector DNA in which it is inserted for cloning or expression, and it is heterologous to a host cell containing such a vector, in which it is expressed, e.g., a yeast cell.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e., any kind of mutant.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667). Such proteins (and their encoding genes) have sequence homology, as reflected by their sequence similarity, whether in terms of percent similarity or the presence of specific residues or motifs at conserved positions.

Accordingly, the term “sequence similarity” in all its grammatical forms refers to the degree of identity or correspondence between nucleic acid or amino acid sequences of proteins that may or may not share a common evolutionary origin (see Reeck et al., supra). However, in common usage and in the instant application, the term “homologous,” when modified with an adverb such as “highly,” may refer to sequence similarity and may or may not relate to a common evolutionary origin.

In a specific embodiment, two DNA sequences are “substantially homologous” or “substantially similar” when at least about 80%, and most preferably at least about 90 or 95%) of the nucleotides match over the defined length of the DNA sequences, as determined by sequence comparison algorithms, such as BLAST, FASTA, DNA Strider, etc. An example of such a sequence is an allelic or species variant of the specific Ulp1 genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc)

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SCC. High stringency hybridization conditions correspond to the highest Tₘ, e.g., 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2XSSC, at 42° C. in 50% formamide, 4XSSC, or under conditions that afford levels of hybridization equivalent to those observed under either of these two conditions.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of Ulp1, or to detect the presence of nucleic acids encoding Ulp1. In a further embodiment, an oligonucleotide of the invention can form a triple helix with a Ulp1 DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

Ulp1 Nucleic Acids

A gene encoding mutant Ulp1, whether genomic DNA or cDNA, can be isolated from any source, particularly from a human cDNA or genomic library. Methods for obtaining Ulp1 gene are well known in the art, as described above (see, e.g., Sambrook et al., 1989, supra). The DNA may be obtained by standard procedures known in the art from cloned DNA (e.g., a DNA “library”), and preferably is obtained from a cDNA library prepared from tissues with high level expression of the protein, by chemical synthesis, by cDNA cloning, or by the cloning of genomic DNA, or fragments thereof, purified from the desired cell (See, for example, Sambrook et al., 1989, supra; Glover, D. M. (ed.), 1985, DNA Cloning: A Practical Approach, MRL Press, Ltd., Oxford, U.K. Vol. I, II). Clones derived from genomic DNA may contain regulatory and intron DNA regions in addition to coding regions; clones derived from cDNA will not contain intron sequences. Whatever the source, the gene should be molecularly cloned into a suitable vector for propagation of the gene. Identification of the specific DNA fragment containing the desired Ulp1 gene may be accomplished in a number of ways. For example, a portion of an Ulp1 gene exemplified infra can be purified and labeled to prepare a labeled probe, and the generated DNA may be screened by nucleic acid hybridization to the labeled probe (Benton and Davis, Science 196:180, 1977; Grunstein and Hogness, Proc. Natl. Acad. Sci. U.S.A. 72:3961, 1975). Those DNA fragments with substantial homology to the probe, such as an allelic variant from another individual, will hybridize.

Further selection can be carried out on the basis of the properties of the gene, e.g., if the gene encodes a protein product having the isoelectric, electrophoretic, amino acid composition, partial or complete amino acid sequence, antibody binding activity, or ligand binding profile of Ulp1 protein as disclosed herein. Thus, the presence of the gene may be detected by assays based on the physical, chemical, immunological, or functional properties of its expressed product.

Other DNA sequences which encode substantially the same amino acid sequence as an Ulp1 gene may be used in the practice of the present invention. These include but are not limited to allelic variants, species variants, sequence conservative variants, and functional variants.

Amino acid substitutions may also be introduced to substitute an amino acid with a particularly preferable property. For example, a Cys may be introduced a potential site for disulfide bridges with another Cys.

The genes encoding Ulp1 derivatives and analogs of the invention can be produced by various methods known in the art. The manipulations which result in their production can occur at the gene or protein level. For example, the cloned Ulp1 gene sequence can be modified by any of numerous strategies known in the art (Sambrook et al., 1989, supra). The sequence can be cleaved at appropriate sites with restriction endonuclease(s), followed by further enzymatic modification if desired, isolated, and ligated in vitro. In the production of the gene encoding a derivative or analog of Ulp1, care should be taken to ensure that the modified gene remains within the same translational reading frame as the Ulp1 gene, uninterrupted by translational stop signals, in the gene region where the desired activity is encoded.

Additionally, the Ulp1 encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Such modifications can be made to introduce restriction sites and facilitate cloning the Ulp1 gene into an expression vector. Any technique for mutagenesis known in the art can be used, including but not limited to, in vitro site-directed mutagenesis (Hutchinson, C., et al, J. Biol. Chem. 253:6551, 1978; Zoller and Smith, DNA 3:479-488, 1984; Oliphant et al., Gene 44:177, 1986; Hutchinson et al., Proc. Natl. Acad. Sci. U.S.A. 83:710, 1986), use of TAB″ linkers (Pharmacia), etc. PCR techniques are preferred for site directed mutagenesis (see Higuchi, 1989, “Using PCR to Engineer DNA”, in PCR Technology: Principles and Applications for DNA Amplification, H. Erlich, ed., Stockton Press, Chapter 6, pp. 61-70).

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences.

Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences form the yeast 2μ plasmid.

Expression of Ulp1 Polypeptides

The nucleotide sequence coding for Ulp1, or antigenic fragment, derivative or analog thereof, or a functionally active derivative, including a chimeric protein, thereof, can be inserted into an appropriate expression vector, i.e., a vector which contains the necessary elements for the transcription and translation of the inserted protein-coding sequence. Thus, a nucleic acid encoding Ulp1 of the invention can be operationally associated with a promoter in an expression vector of the invention. Both cDNA and genomic sequences can be cloned and expressed under control of such regulatory sequences. Such vectors can be used to express functional or functionally inactivated Ulp1 polypeptides.

The necessary transcriptional and translational signals can be provided on a recombinant expression vector, or they may be supplied by the native gene encoding Ulp1 and/or its flanking regions.

Potential host-vector systems include but are not limited to mammalian cell systems transfected with expression plasmids or infected with virus (e.g., vaccinia virus, adenovirus, adeno-associated virus, herpes virus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

Expression of Ulp1 protein may be controlled by any promoter/enhancer element known in the art, but these regulatory elements must be functional in the host selected for expression. Promoters which may be used to control Ulp1 gene expression include, but are not limited to, cytomegalovirus (CMV) promoter, the SV40 early promoter region (Benoist and Chambon, 1981, Nature 290:304-310), the promoter contained in the 3′ long terminal repeat of Rous sarcoma virus (Yamamoto, et al., Cell 22:787-797, 1980), the herpes thymidine kinase promoter (Wagner et al., Proc. Natl. Acad. Sci. U.S.A. 78:1441-1445, 1981), the regulatory sequences of the metallothionein gene (Brinster et al, Nature 296:39-42, 1982); prokaryotic expression vectors such as the β-lactamase promoter (Villa-Komaroff, et al., Proc. Natl. Acad. Sci. U.S.A. 75:3727-3731, 1978), or the tac promoter (DeBoer, et al., Proc. Natl. Acad. Sci. U.S.A. 80:21-25, 1983); see also “Useful proteins from recombinant bacteria” in Scientific American, 242:74-94, 1980; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit tissue specificity, particularly endothelial cell-specific promoters.

Soluble forms of the protein can be obtained by collecting culture fluid, or solubilizing inclusion bodies, e.g., by treatment with detergent, and if desired sonication or other mechanical processes, as described above. The solubilized or soluble protein can be isolated using various techniques, such as polyacrylamide gel electrophoresis (PAGE), isoelectric focusing, 2-dimensional gel electrophoresis, chromatography (e.g., ion exchange, affinity, immunoaffinity, and sizing column chromatography), centrifugation, differential solubility, immunoprecipitation, or by any other standard technique for the purification of proteins.

Vectors

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMal-C2, pET, pGEX (Smith et al., Gene 67:31-40, 1988), pMB9 and their derivatives, plasmids such as RP4; phage DNAS, e.g., the numerous derivatives of phage 1, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Preferred vectors for introducing Ulp1 coding sequences into mammalian or insect cells are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant Ulp1 protein or polypeptide domain fragment thereof can be introduced using a viral vector or through direct introduction of DNA.

Viral vectors commonly used for targeting procedures are DNA-based vectors and retroviral vectors. Methods for constructing and using viral vectors are known in the art (see, e.g., Miller and Rosman, BioTechniques, 7:980-990, 1992). Preferably, the viral vectors are replication defective, that is, they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome which are necessary for encapsidating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV 1) vector (Kaplitt et al., Molec. Cell. Neurosci.2:320-330,1991), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 90:626-630, 1992; see also La Salle et al., Science 259:988-990, 1993); and a defective adeno-associated virus vector (Samulski et al., J. Virol. 61:3096-3101, 1987; Samulski et al., J. Virol. 63:3822-3828, 1989; Lebkowski et al., Mol. Cell. Biol. 8:3988-3996, 1988).

Various companies produce viral vectors commercially, including but by no means limited to Avigen, Inc. (Alameda, Calif; AAV vectors), Cell Genesys (Foster City, Calif; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

In another embodiment, the vector can be introduced by lipofection, as naked DNA, or with other transfection facilitating agents (peptides, polymers, etc.). Synthetic cationic lipids can be used to prepare liposomes for in vivo transfection of a gene encoding a marker (Felgner, et. al., Proc. Natl. Acad. Sci. U.S.A. 84:7413-7417, 1987; Felgner and Ringold, Science 337:387-388, 1989; see Mackey, et al., Proc. Natl. Acad. Sci. U.S.A. 85:8027-8031, 1988; Ulmer, et al., Science 259:1745-1748, 1993). Useful lipid compounds and compositions for transfer of nucleic acids are described in International Patent Publications WO95/18863 and WO96/17823, and in U.S. Pat. No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically.

Other molecules are also useful for facilitating transfection of a nucleic acid, such as a cationic oligopeptide (e.g., International Patent Publication WO95/21931), peptides derived from DNA binding proteins (e.g., International Patent Publication WO96/25508), or a cationic polymer (e.g, International Patent Publication WO95/21931).

Alternatively, non-viral DNA vectors can be introduced into the desired host cells by methods known in the art, e.g., electroporation, microinjection, cell fusion, DEAE dextran, calcium phosphate precipitation, use of a gene gun (ballistic transfection; see, e.g., U.S. Pat. Nos. 5,204,253, 5,853,663, 5,885,795, and 5,702,384 and see Sanford, TIB-TECH, 6:299-302, 1988; Fynan et al.,Proc. Natl. Acad. Sci. U.S.A., 90:11478-11482,1993; and Yang et al., Proc. Natl. Acad. Sci. U.S.A., 87:1568-9572, 1990), or use of a DNA vector transporter (see, e.g., Wu, et al., J. Biol. Chem. 267:963-967, 1992; Wu and Wu, J. Biol. Chem. 263:14621-14624, 1988; Hartmut, et al., Canadian Patent Application No. 2,012,31 1, filed Mar. 15, 1990; Williams, et al., Proc. Natl. Acad. Sci. USA 88:2726-2730, 1991). Receptor-mediated DNA delivery approaches can also be used (Curiel, et al., Hum. Gene Ther. 3:147-154, 1992; Wu and Wu, J. Biol. Chem. 262:4429-4432, 1987).

Protease-Substrate Complex Formation

The present invention describes a composition comprising a polypeptide of a SUMO protease catalytic domain in a trapped proteolytic deacylation intermediate complex with its substrate. The composition may then be treated with methods known in the art to produce a crystalline structure of the complex. The present invention contemplates characterization of the structure of any protease that produces deacylation intermediates upon interaction with a substrate, particularly any cysteine protease, e.g., by trapping the protease-substrate covalent intermediate. Specifically, the present invention contemplates a composition where the SUMO protease is Ulp1. Ulp1 may be derived from various sources, including, but not limited to, mammal, yeast, insects, and plants. In one preferred embodiment, the Ulp1 is derived from mammal, specifically human. The composition may include any substrate of Ulp1, such as SUMO and Smt3. In a specific embodiment, the substrate is Smt3.

The complexed molecules in the composition are preferably obtained in a crystalline structure. The complexed molecules may be formed into a crystalline structure using techniques that are well known in the art, where the crystallizing conditions are modified and optimized for crystal formation of specific proteases and their substrate. Alternatively, other spectroscopic techniques for structure evaluation, such as nuclear magnetic resonance, Raman spectroscopy, circular dichroism, and neutron diffraction can be used to study trapped protease-substrate complexes.

The coordinates of atoms and structure of the protease and substrate in the crystal structure may be defined using any method and computational method needed. For example, if the protease and substrate complex are in a liquid form, then nuclear magnetic resonance can be used to determine the coordinates and structure of the complex. For complexes that are in a crystalline structure x-ray crystallography may be used to elucidate the structure.

The invention also describes a method of preparatively trapping and purifying large amounts of the proteolytic deacylation intermediate using a reducing agent. The method of trapping the proteolytic deacylation intermediate involves (a) combining the protease and substrate in a molar ratio, (b) adding a reducing agent that is capable of trapping the substrate in an intermediate state with the protease, and (c) then adjusting the pH of the solution to about 7.0. The invention allows for performing steps (a) and (b) in any order or simultaneously. In a specific embodiment the protease is a SUMO protease, and more specifically the SUMO protease is the catalytic domain of Ulp1. Ulp1 may be derived from various sources, including, but not limited to, mammal, yeast, insects, and plants. In one preferred embodiment, the Ulp1 is derived from mammal, specifically human. The complex includes any substrate of Ulp1, such as SUMO and Smt3. In a specific embodiment, the substrate is Smt3.

The pH of the solution may be adjusted to about 7.0 by various methods known in the art, such as adding the required amount of an acid or base to the solution to adjust the pH to about 7.0. Other methods that may be used to adjust the pH of a solution, as appropriate for the system, include, but not limited to, chromatography and dialysis. In one embodiment of the invention, the pH of the solution is adjusted by dialysis. The protease-substrate complex may then be isolated from the solution after the pH is adjusted by dialysis. The protease-substrate complex may be isolated from the solution using any methods known in the art, including high performance liquid chromatography, size exclusion chromatography, dialysis, fast protein liquid chromatography, and crystallization. Other method of isolating the complex may be used, depending on the protease and substrate.

The molar ratio of protease to substrate may encompass any ratio where the concentration of substrate is in excess to insure that the protease may form a complex with it. Theoretically, the substrate may be in infinite excess to the protease, however that would lead to significant waste of substrate that remains uncomplexed to the protease. In the present invention the molar ratio of the protease to substrate ranges from 1:1 to 1:5. More preferred, the Ulp1 and Smt3 are in a 1:3 molar ratio.

The protease and substrate are trapped in a deacylation complex by addition of a reducing agent in an amount that is effective to trap the proteins. An effective amount of reducing agent is defined as the amount needed to trap an isolatable amount of the complex in the solution. Any reducing agent is contemplated by the present invention. However, several reducing agents, such as lithium aluminum hydride, could reduce other portions of the protease and substrate in the complex. In one specific embodiment the reducing agent is sodium borohydride.

Screening and Chemistry

According to the present invention, the structure of a Ulp1-Smt3 complex, or other trapped protease-substrate complexes, are useful to identify drugs that are effective in treating disorders associated with Ub/Ubl pathway, including tumorogenesis and acute promyelocytic leukemia.

Rational Drug Design

The invention also defines a method of identifying novel substrates for cysteine proteases by using rational drug design methods. The invention allows for designing substrates that would interact with amino acids in the protease catalytic site as defined by computer molecular modeling methods based on the crystal structure of the protease and other substrates. A substrate may include a competitive inhibitor, analog of the native substrate, or a suicide inhibitor. The designed substrates would be designed so that they interact with amino acid residues that are defined to be (i) necessary for interaction with a substrate and (ii) protease activity.

The present invention contemplates evaluating potential compounds for covalent and non-covalent interactions between the protease and substrate. Computer modeling methods that may be used to evaluate these interactions include, but are not limited to, SYBYL and Monte Carlo computer programs. The present invention contemplates computer algorithms that evaluate bonded and non-bonded interactions between the protease and the substrate. Bonded interactions that may be evaluated include, but are not limited to, bond stretching, rotational strain, and torsional strain. Non-bonded interactions that may be evaluated include van Der Waals forces, hydrogen bonds and dipole-dipole interaction.

In particular, identification of putative Ulp binding compounds provides for development of screening assays, particularly for high throughput screening of molecules that up-or down-regulate the activity of Ulp1, e.g., by permitting expression of Ulp1 in quantities greater than can be isolated from natural sources, or in indicator cells that are specially engineered to indicate the activity of Ulp1 expressed after transfection or transformation of the cells.

Any screening technique known in the art can be used to screen for Ulp1 agonists or antagonists. The present invention contemplates screens for small molecule ligands or ligand analogs and mimics, as well as screens for natural ligands that bind to and agonize or antagonize Ulp1 expression activity in vivo. For example, natural products libraries can be screened using assays of the invention for molecules that agonize or antagonize Ulp1 expression or activity.

Another approach uses recombinant bacteriophage to produce large libraries. Using the “phage method” (Scott and Smith, Science 249:386-390, 1990; Cwirla, et al., Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In another aspect, synthetic libraries (Needels et al., Proc. Natl. Acad. Sci. USA 90:10700-4, 1993; Ohlmeyer et al., Proc. Natl. Acad. Sci. USA 90:10922-10926, 1993; Lam et al., International Patent Publication No. WO92/00252; Kocis et al., International Patent Publication No. WO9428028) and the like can be used to screen for Ulp1 ligands according to the present invention.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, NH), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., Tib Tech, 14:60, 1996).

Knowledge of the crystal structure of Ulp1, particularly when trapped with a substrate, can provide an initial clue as the inhibitors or antagonists of the protein. Moreover, based on sequence and enzymatic activity similarities of Ulp1 to other cysteine proteases, particularly cell-cycle associated cysteine proteases, the crystal structure of Ulp1 permits development of classes of cysteine protease inhibitors. Identification and screening of antagonists is further facilitated by determining structural features of the protein, e.g., using X-ray crystallography, neutron diffraction, nuclear magnetic resonance spectrometry, and other techniques for structure determination. These techniques provide for the rational design or identification of agonists and antagonists.

In vivo Screening Methods

Intact cells or whole animals expressing a gene encoding Ulp1 can be used in screening methods to identify candidate drugs.

In one series of embodiments, a permanent cell line is established. Alternatively, cells (including without limitation mammalian, insect, yeast, or bacterial cells) are transiently programmed to express an Ulp1 gene by introduction of appropriate DNA or mRNA. Identification of candidate compounds can be achieved using any suitable assay, including without limitation (i) assays that measure selective binding of test compounds to Ulp1 (ii) assays that measure the ability of a test compound to modify (i.e., inhibit or enhance) a measurable activity or function of Ulp1 and (iii) assays that measure the ability of a compound to modify (i.e., inhibit or enhance) the transcriptional activity of sequences derived from the promoter (i.e., regulatory) regions the Ulp1 gene.

High-Throughput Screen

Agents according to the invention may be identified by screening in high-throughput assays, including without limitation cell-based or cell-free assays. It will be appreciated by those skilled in the art that different types of assays can be used to detect different types of agents. Several methods of automated assays have been developed in recent years so as to permit screening of tens of thousands of compounds in a short period of time. Such high-throughput screening methods are particularly preferred. The use of high-throughput screening assays to test for agents is greatly facilitated by the availability of large amounts of purified polypeptides, as provided by the invention.

### EXAMPLES

The present invention will be better understood by reference to the following Examples, which are provided as exemplary of the invention, and not by way of limitation.

Materials and Methods

Protein Purification

A Ulp1 amino acid fragment, from amino acids 403-621 (Ulp1 (403-621)), was amplified by polymerase chain reaction. The PCR fragment was digested with Nhe1 and Xho1 restriction endonucleases and then ligated into a pET-28b vector (Novagen). E. coli BL21 (DE3)pLysS bacterial cells were transformed with Ulp1 (403-621). Cells were grown at 37° C. for 20-24 hours. Cell cultures then were grown at 37° C. in superbroth, containing 50 mg/ml kanamycin and 25 mg/ml chloamphenicol, by fermentation. When the A₆₀₀ of the culture reached about 2.0 the culture was cooled to 30° C., adjusted to 1.0 mM IPTG, and fermented for about 4 hours at 30° C. Remaining steps were performed at 4° C. The culture was centrifuged and the cell pellet was resuspended in 200 mL lysis buffer (50 mM Tris-HCl, 0.15 mM NaCl, 20% (w/v) sucrose; pH=8.0) and sonicated. Insoluble material was removed by centrifugation and the soluble fraction was placed over 10 mL of Ni-NTA-agarose resin (Qiagen) and washed with buffer A (50 mM Tris-HCl, 0.5 M NaCl, 20 mM imidazole). The protein was eluted from the resin with a gradient (20-300 mM imidazole) in buffer A. Peak fractions of His-tagged Ulp1 (403-621)p were pooled and dialyzed against 50 mM Tris-HCl (pH=8.0) containing 200 mM NaCl, 2 mM β-mercaptoethanol, and 100 units bovine thrombin (Sigma) for about 20-24 hours.

A Smt3 PCR fragment (Smt3(13-101)p) was digested with Nco1 and Xho1 restriction endonucleases and then ligated into a pET-28b vector. E. coli BL21 (DE3)pLysS bacterial cells were transformed with Smt3(13-101)p. Expression and purification of the protein was performed following the same protocol as described for Ulp1 (403-621)p, with the exception that the protein was dialyzed against 50 mM Tris-HCl (pH=8.0) containing 200 mM NaCl.

Sumo (??-101)p was prepared in a similar manner to that described for Smt3(13-101)p.

Selenomethionyl-containing Ulp1 (403-621) was expressed in DL41 cells (methionine auxotrophe) that contained the bacteriophage T7 polymerase (Hendrickson et al., EMBO J., 1990, 9:1665-1672).

The Smt3-GFPuv fusion protein was constructed by ligating SMT3 into the pGFPuv vector (Clontech). Smt3-GFPuv was overexpressed in JM109 E. coli strain. Purification was attained by standard chromatographic techniques and included passes over size exclusion, anion-exchange resins, and finally, application to a MonoQ column (Pharmacia). The Smt3-GFP moiety was followed during purification by analyzing fluorescence over a standard UV transilluminator.

His6-ubiquitin-Smt3-HA was expressed from pQE30 (Li and Hochstrasser) in JM109 E. coli. The histidine-tagged fusion protein (His6-ubiquitin-Smt3-HA) was placed over Ni-agarose resin, washed and eluted as previously described. The pooled fractions were then placed onto a gel filtration column (Superdex 75 prep-grade) for size exclusion. These fractions were pooled and placed onto a MonoQ column (Pharmacia) and eluted with a 0.02-1M gradient of NaCl. Smt3-GFP, Sumo-His, and His6-ubiquitin-Smt3-HA were concentrated to ˜10 mg/ml and frozen at −80° C.

Proteolysis Assays

Proteolysis assays were prepared by incubating a solution at about 37° C. that contained about 1.0 mg/ml substrate and about 10⁻³-10⁻⁶ mg/ml Ulp1 (403-621)p in buffer containing 20 mM Tris-HCl (pH=8.0) and 150 mM NaCl. The Smt3-GFP reaction was subsequently analyzed by native-gel electrophoresis. The reactions containing Sumo-His and His6-ubiquitin-Smt3-HA were analyzed by SDS-PAGE.

Synthesis of Covalent Adduct Between Ulp1 (403-621)p and Smt3(13-101)p

Ulp1(403-621)p and Smt3(13-101)p were placed in a beaker at a 1:3 molar ratio with stirring. Then 5 aliquots of sodium borohydride were stirred into the beaker over 30 minutes to a final concentration of 50 mM. The mixture was dialyzed against 20 mM Tris-HCl (pH=8.0) containing 20 mM NaCl at 4° C. The dialyzed solution was then loaded onto a Mono-Q column (Qiagen). The Ulp1(403-621)p-Smt3(13-101)p complex was eluted from the column with 0.02-0.5 M NaCl gradient. Fractions were analyzed by native gel electrophoresis. Ulp1 (403-621)p-Smt3(13-101)p was purified further by size exclusion chromatography on a Superdex 75 prep-grade column (Pharmacia). Fractions were pooled, concentrated to about 3.5 mg/mL, and stored in 20 mM Tris-HCl containing 50 mM NaCl at −80° C.

Crystallization and Data Collection

Crystals were grown at 21° C. by the hanging drop vapor diffusion method. Ulp1 (403-621)p-Smt3(13-101)p complex was mixed with an equal volume of reservoir buffer containing 0.1 M MES (pH=6.5), 10% (w/v) polyethylene glycol 20000, and 3% (w/v) 1,6-hexandiol. Crystals were grown over several days to a size of 0.05×0.1×0.3 mm. Crystals were stabilized in reservoir solution for heavy atoms soaks and pre-incubated in reservoir solution plus 17% ethylene glycol. X-ray data used to collect native and heavy atom sets were collected using crystals at a laboratory copper Kα source (Rigaku RU200) equipped with a confocal optics and a Raxis-IV imaging plate detector system. The high-resolution native data set and a four-wavelength MAD data set were collected at the National Synchrotron Light Source (Brookhaven, N.Y.) at beamline X4A on an ADSC Quantum-4 detector. Data were processed using DENZO and SCALEPACK. Ulp1 (403-621)p-Smt3(13-101)p were crystallized in space group P21212 (a=125.8 Å, b=53.4 Å, c=54.3 Å, α=β=γ=90°).

Structure Determination and Refinement

2.6 Åelectron density maps for the Ulp1 (403-621)p-Smt3(13-101)p complex were initially derived using multiple isomorphous replacement (MIR) methods and solvent flattening (DM). Mercury sites were identified using Patterson methods and placed into MLPHARE for phase refinement. An initial trace into these maps was accomplished using the program O and the model was not refined. A selenomethionyl-derived Ulp1 (403-625)p and native Smt3(11-97)p crystal was subjected to a four-wavelength MAD experiment (Hendrickson, et al., EMBO J., 1991, 9:1665-1672). The 1.8 Å MAD data was input into the program SOLVE (Terwilliger and Berendzen, Acta Crystallogr, 1999, D55:849-861). Six selenium sites were located and used to generate 1.8 Å phases. Solvent flattening was performed using the program DM (Cowtan, Joint CCP4 and ESF-EACBM Newsletter on Protein Crystallography, 1994, 31:34-38). The initial model derived from the MIR electron density was placed into the MAD electron density maps for manual building. Refinement was accomplished using the programs REFMAC (Murshudov et al., Acta. Crystallogr., 1997, A50:157-163) and wARP/ARP (Lamzin and Wilson, Acta. Crystallogr., 1993, D49:129-149) for automated building and placement of waters. The model underwent three rounds of refinement at 1.8 Å with manual rebuilding between each round. Refinement was extended to 1.6 Å for another round. The current model contains 735 waters and 303 amino acids (Rfree=25.3, R=17.9) and has excellent geometry; 92.9% of residues are in the most favored region of the Ramachandran plot with none occurring in disallowed regions. The model contained all 219 amino acid residues of Ulp1 (403-621)p plus two additional N-terminal amino acids that correspond to residues left behind after thrombin cleavage of the N-terminally HIS-tagged protein. Ulp1p Arg422 did not have adequate density to model its side chain. The model for Smt3(13-98)p contains 79 of the possible 87 amino acid residues. Electron density for N-terminal Smt3 residues 12 through 19 could not be observed and were left out of the model.

Yeast Plasmids and Strains

Standard techniques were used to grow and maintain yeast strains (Guthrie and Fink, Methods Enzymol., 1991; 194). Yeast strain W303-1A (MATa ura3-1 ade2-1 trp1-1 his3, -1115, leu2-3, -112 can1-100) was used to generate a Ulp1 null strain by one-step gene replacement procedure (Rothstein, Methods Enzymol., 1991; 194:281-301). The resulting strain was termed Ulp1 Δ strain W303ULP1 (MATa ura3-1 ade2-1 tr1-1 his3, -1115, leu 2-3, -112 can1-100, Ulp1 ::LEU2, pSE360-ULP1). In vivo complementation analysis was conducted using plasmid shaffle technique (Boeke et al., Methods Enzymol., 1987; 154,164). W303ULP1 was transformed with pSE358 plasmid containing either wildtype or mutant Ulp1 under Ulp1 natural promoter. Yeast cells were streaked onto agar plates that lack tryptophan and incubated at about 30° C. until yeast colonies were visible. Colonies were then restreaked onto agar plates containing and 5-fluoro-orotic acid to induce the loss of pSE360-ULP1.

Glacatose Inducible Expression

Wildtype or deletion mutant Ulp1 fragments were amplified by PCR. The fragments were digested with EcoR1 and BamH1 restriction endonucleases and ligated into PYX133 (CEN TRP1 GAL promoter; Ingenius). W303-1A was transformed with the plasmid. Yeast strains were grown on plates lacking tryptophan, to select for cells that contain the plasmid of interest, at about 30° C. Positive yeast colonies were then streaked onto SD(-Trp) agar plates, containing 2% (w/v) raffinose, with and without 0.01% (w/v) galactose. Cells were incubated at 30° C. until a color change in the yeast colonies was observed.

Point mutations were introduced to subcloned fragments using QuikChange Site-Directed Mutagenesis Kit (Qiagen).

Results

Ulp1 (403-621)p displayed full catalytic activity in cleavage reactions with human SUMO-1 and yeast Smt3 to produce their respective mature protein forms (data not shown). Ulp1 (403-621)p also showed full catalytic activity in deconjugation reactions with Smt3-GFPuv and His6-ubiquitin-Smt3-HA (data not shown), suggesting that the C-terminal fragment of Ulp1 (Ulp1 (403-621)p) is able to (i) deconjugate large proteins and (ii) produce the mature forms of Smt3 and SUMO.

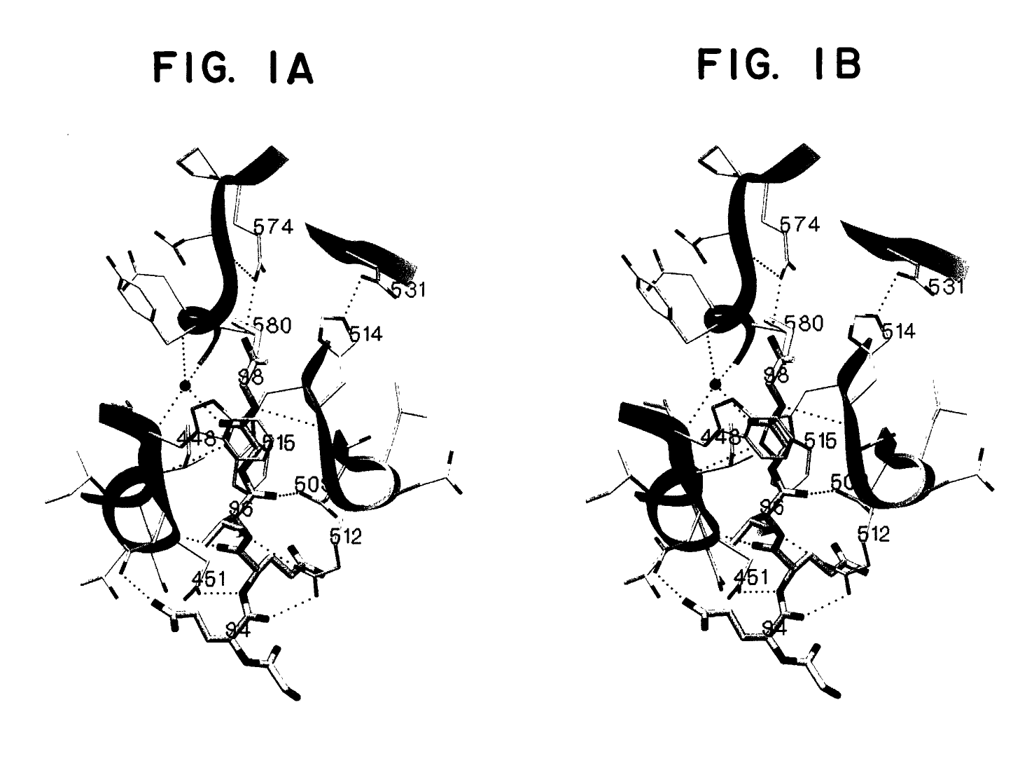

The secondary structure of Ulp1 (403-621)p includes 7α helices and 7β strands. Ulp1 (403-621)p exhibits structural similarities to other papain-like cysteine proteases in the active site, which includes the central a helix, 3β strands, and the catalytic triad (Cys-His-Glu/Asp) (FIG. 1).

The Ulp1(403-621)p-Smt3(13-98)p structure is currently refined to 1.6 angstroms and includes all 219 amino acid residues of Ulp1(403-621)p and 79 of the possible 87 amino acid residues of Smt3(13-98)p. Coordinates of the Ulp1(403-621)p-Smt3(13-98)p structure are shown in Table 1 (present before the claims). The interaction between Ulp1 and Smt3 is described below in the context of six conserved Ulp1 motifs.

The interactions of motif 1 of Ulp1 with Smt3 include side-chain to main-chain hydrogen bonding, Van der Waals (VDW) contacts, and one salt bridge between Arg438 of Ulp1 and the Glu94 of Smt3.

Motif 2 of Ulp1 is involved in the recognition of residues at the C terminus of Smt3, including VDW contacts with the Gly-Gly motif of Smt3 by Trp448. Motif 2 also contributes (i) 3 hydrogen bonds to the main-chain of Smt3 strand 5 and (ii) 2 acidic residues (Glu455 and Asp451) that participate in salt-bridging interactions with 2 Smt3 basic residues (Arg64 and Arg71).

Motif 3 of Ulp1 provides direct hydrogen bonds to the main-chain of Smt3, Gly69, by a conserved Asn472. Hydrogen bonds are also made between Ser473 and Thr477 to an invariant Smt3 residue Gln95 in strand 5. VDW contacts in the Smt3-Ulp1 interface is contributed by Phe474.

Motif 4 of Ulp1 contains salt bridging moieties (Arg489 and Arg493) that make contact with two acidic residues in Smt3 (Asp68 and Asp82). Positions 489 and 493 also produce hydrogen bonds through water to make interactions with Asp87 in Smt3. Motif 4 contributes VDW contacts in the Ulp1-Smt3 interface through Trp490.

Motif 5 of Ulp1 contains His514, which is the general base of the catalytic triad. Motif 5 also participates in several interactions with the C-terminal tail of Smt3. Motif 5 contains Asn509 and Gln512, which participate in hydrogen bonds with main-chain atoms of Smt3 residues 94 and 96. The carbonyl of Ser513 is involved in a main-chain to main-chain hydrogen bond with Smt3 Gly98.

Motif 6 of Ulp1 contains active site amino acid residues Cys580 and Gln574, which are involved in the function of the protein.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that values are approximate, and are provided for description.

Patents, patent applications, publications, procedures, and the like are cited throughout this application, the disclosures of which are incorporated herein by reference in their entireties.

TABLE 1

| | | | |
|---|---|---|---|
| HEADER | HYDROLASE | 17-APR-00 | 1EUV |

| TITLE | X-RAY STRUCTURE OF THE C-TERMINAL ULP1 PROTEASE DOMAIN IN | |
|---|---|---|
| TITLE | 2 COMPLEX WITH SMT3, THE YEAST ORTHOLOG OF SUMO. | |
| COMPND | MOL_ID: 1; | |
| COMPND | 2 MOLECULE: ULP1 PROTEASE; | |
| COMPND | 3 CHAIN: A; | |
| COMPND | 4 FRAGMENT: C-TERMINAL PROTEASE DOMAIN; | |
| COMPND | 5 ENGINEERED: YES; | |
| COMPND | 6 MOL_ID: 2; | |
| COMPND | 7 MOLECULE: UBITQUTIN-LIKE PROTEIN SMT3; | |
| COMPND | 8 CHAIN: B; | |
| COMPND | 9 FRAGMENT: SMT3 RESIDUES 13-98; | |
| COMPND | 10 ENGINEERED: YES | |
| SOURCE | MOL_ID: 1; | |
| SOURCE | 2 ORGANISM_SCIENTIFIC: SACCHAROMYCES CEREVISIAE; | |
| SOURCE | 3 ORGANISM_COMMON: YEAST; | |
| SOURCE | 4 EXPRESSION_SYSTEM: ESCHERICHIA COLI; | |
| SOURCE | 5 EXPRESSION_SYSTEM_COMMON: BACTERIA; | |
| SOURCE | 6 EXPRESSION_SYSTEM_PLASMID: PET28B; | |
| SOURCE | 7 MOL_ID: 2; | |
| SOURCE | 8 ORGANISM_SCIENTIFIC: SACCHAROMYCES CEREVISIAE; | |
| SOURCE | 9 ORGANISM_COMMON: YEAST; | |
| SOURCE | 10 EXPRESSION_SYSTEM: ESCHERICHIA COLI; | |
| SOURCE | 11 EXPRESSION_SYSTEM_COMMON: BACTERIA; | |
| SOURCE | 12 EXPRESSION_SYSTEM_PLASMID: PET28B | |
| KEYWDS | SUMO HYDROLASE, UBIQUITIN-LIKE PROTEASE 1, SMT3 HYDROLASE | |
| KEYWDS | 2 DESUMOYLATING ENZYME, CYSTEINE PROTEASE, SUMO PROCESSING | |
| KEYWDS | 3 ENZYME, SMT3 PROCESSING ENZYME, NABH4, THIOHEMIACETAL, | |
| KEYWDS | 4 COVALENT PROTEASE ADDUCT | |
| EXPDTA | X-RAY DIFFRACTION | |
| AUTHOR | E.MOSSESSOVA,C.O.LIMA | |
| REVDAT | 1 07-JUN-00 1EUV | 0 |

| JRNL | AUTH | E.MOSSESSOVA,C.D.LIMA | | |
|---|---|---|---|---|
| JRNL | TITL | ULP1-SUMO CRYSTAL STRUCTURE AND GENETIC ANALYSIS | | |
| JRNL | TITL 2 REVEAL CONSERVED INTERACTIONS AND A REGULATORY | | | |
| JRNL | TITL 3 ELEMENT ESSENTIAL FOR CELL GROWTH IN YEAST | | | |
| JRNL | REF | MOL. CELL | V. | 5 865 2000 |
| JRNL | REFN | ASTM MOCEFL US ISSN 1097-2765 | | |
| REMARK | 1 | | | |
| REMARK | 1 REFERENCE 1 | | | |
| REMARK | 1 AUTH | S.J.LI,M.HOCHSTRASSER | | |
| REMARK | 1 TITL | A NEW PROTEASE REQUIRED FOR CELL-CYCLE PROGRESSION | | |
| REMARK | 1 TITL 2 IN YEAST. | | | |
| REMARK | 1 REF | NATURE | V. | 398 246 1999 |

| | | |
|---|---|---|
| REMARK | 1 REFN | ‘ASTM NATUAS UK ISSN 0028-0836 |

| REMARK | 2 | |
|---|---|---|
| REMARK | 2 RESOLUTION. 1.6 ANGSTROMS. | |
| REMARK | 3 | |
| REMARK | 3 REFINEMENT. | |
| REMARK | 3 PROGRAM | : REFMAC |
| REMARK | 3 AUTHORS | : MURSHUDOV,VAGIN,DODSON |
| REMARK | 3 | |

| REMARK | 3 DATA USED IN REFINEMENT. | | |
|---|---|---|---|
| REMARK | 3 RESOLUTION RANGE HIGH | (ANGSTROMS) | : 1.60 |
| REMARK | 3 RESOLUTION RANGE LOW | (ANGSTROMS) | : 25.0 |
| REMARK | 3 DATA CUTOFF | (SIGMA(F)) | : 1.000 |
| REMARK | 3 COMPLETENESS FOR RANGE | (%) | : 96.6 |
| REMARK | 3 NUMBER OF REFLECTIONS | | : 47560 |
| REMARK | 3 | | |

| REMARK | 3 FIT TO DATA USED IN REFINEMENT. | | |
|---|---|---|---|
| REMARK | 3 CROSS-VALIDATION METHOD | | : NULL |
| REMARK | 3 FREE R VALUE TEST SET SELECTION | | : 5% OF THE OBSERVED |
| REMARK | 3 | | DATA |
| REMARK | 3 R VALUE | (WORKING + TEST SET) | 0.193 |
| REMARK | 3 R VALUE | (WORKING SET) | : 0.190 |
| REMARK | 3 FREE R VALUE | | 0.251 |

| | | | |
|---|---|---|---|
| REMARK | 3 FREE R VALUE TEST SET SIZE | (%) | : NULL |
| REMARK | 3 FREE R VALUE TEST SET COUNT | | : 2378 |
| REMARK | 3 | | |

| REMARK | 3 NUMBER OF NON-HYDROGEN ATOMS USED IN REFINEMENT. | |
|---|---|---|
| REMARK | 3 PROTEIN ATOMS | : 2417 |
| REMARK | 3 NUCLEIC ACID ATOMS | : 0 |
| REMARK | 3 HETEROGEN ATOMS | : 0 |
| REMARK | 3 SOLVENT ATOMS | : 432 |
| REMARK | 3 | |
| REMARK | 3 B VALUES. | |

| | | | |
|---|---|---|---|
| REMARK | 3 FROM WILSON PLOT | (A**2) | : 20.69 |

| | | | |
|---|---|---|---|
| REMARK | 3 MEAN B VALUE | (OVERALL, A**2) | : NULL |

| REMARK | 3 OVERALL ANISOTROPIC B VALUE. | |
|---|---|---|
| REMARK | 3 | B11 (A**2) : NULL |
| REMARK | 3 | B22 (A**2) : NULL |
| REMARK | 3 | B33 (A**2) : NULL |
| REMARK | 3 | B12 (A**2) : NULL |
| REMARK | 3 | B13 (A**2) : NULL |
| REMARK | 3 | B23 (A**2) : NULL |
| REMARK | 3 | |

| | |
|---|---|
| REMARK | 3 ESTIMATED OVERALL COORDINATE ERROR. |

| | | | |
|---|---|---|---|
| REMARK | 3 ESU BASED ON R VALUE | (A) : NULL | |
| REMARK | 3 ESU BASED ON FREE R VALUE | (A) : NULL | |
| REMARK | 3 ESU BASED ON MAXIMUM LIKELIHOOD | (A) : NULL | |
| REMARK | 3 | ESU FOR B VALUES BASED ON MAXIMUM LIKELIHOOD | (A**2) : NULL |
| REMARK | 3 | | |
| REMARK | 3 RMS DEVIATIONS FROM IDEAL VALUES. | | |

| | | | |
|---|---|---|---|
| REMARK | 3 DISTANCE RESTRAINTS. | RMS | SIGMA |

| REMARK | 3 | BOND LENGTH | (A) : 0.013 ; NULL |
|---|---|---|---|
| REMARK | 3 | ANGLE DISTANCE | (A) : 1.600 ; NULL |
| REMARK | 3 | INTRAPLANAR 1-4 DISTANCE | (A) : NULL ; NULL |
| REMARK | 3 | H-BOND OR METAL COORDINATION | (A) : NULL ; NULL |
| REMARK | 3 | | |
| REMARK | 3 PLANE RESTRAINT | | (A) : NULL ; NULL |
| REMARK | 3 CHIRAL-CENTER RESTRAINT | | (A**3) : NULL ; NULL |
| REMARK | 3 | | |
| REMARK | 3 NON-BONDED CONTACT RESTRAINTS. | | |
| REMARK | 3 | SINGLE TORSION | (A) : NULL ; NULL |
| REMARK | 3 | MULTIPLE TORSION | (A) : NULL ; NULL |
| REMARK | 3 | H-BOND (X...Y) | (A) : NULL ; NULL |
| REMARK | 3 | H-BOND (X-H...Y) | (A) : NULL ; NULL |
| REMARK | 3 | | |
| REMARK | 3 CONFORMATIONAL TORSION ANGLE RESTRAINTS. | | |
| REMARK | TL,6 3 | SPECIFIED | (DEGREES) : NULL ; NULL |
| REMARK | 3 | PLANAR | (DEGREES) : NULL ; NULL |
| REMARK | 3 | STAGGERED | (DEGREES) : NULL ; NULL |
| REMARK | 3 | TRANSVERSE | (DEGREES) : NULL ; NULL |
| REMARK | 3 | | |

| | | | |
|---|---|---|---|
| REMARK | 3 ISOTROPIC THERMAL FACTOR RESTRAINTS. | RMS | SIGMA |

| | | | |
|---|---|---|---|
| REMARK | 3 MAIN-CHAIN BOND | (A**2) NULL ; NULL | |
| REMARK | 3 MAIN-CHAIN ANGLE | (A**2) : NULL | ; NULL |
| REMARK | 3 SIDE-CHAIN BOND | (A**2) : NULL | ; NULL |
| REMARK | 3 SIDE-CHAIN ANGLE | (A**2) : NULL | ; NULL |
| REMARK | 3 | | |

| REMARK | 3 OTHER REFINEMENT REMARKS: USED A -LOGLIKELIHOOD RESIDUAL | | |
|---|---|---|---|
| REMARK | 3 DERIVED FROM RICE DISTRIBUTION FOR CENTRIC AND ACENTRIC | | |
| REMARK | 3 CASES OF FS SPARSE MATRIX PROCEDURE WITH ANISOTROPIC B- | | |
| REMARK | 3 FACTOR REFINEMENT. | | |
| REMARK | 4 | | |
| REMARK | 4 1EUV COMPLIES WITH FORMAT V. 2.3, 09-JULY-1998 | | |
| REMARK | 6 | | |
| REMARK | 6 COVALENT ADDUCT FORMED BETWEEN THE PROTEOLYTIC | | |
| REMARK | 6 ACTIVE SITE THIOL AND THE C-TERMINAL GLYCINE OF | | |
| REMARK | 6 SMT3 USING THE REDUCING AGENT NABH4. | | |
| REMARK | 100 | | |
| REMARK | 100 THIS ENTRY HAS BEEN PROCESSED BY RCSB ON 20-APR-2000 | | |
| REMARK | 100 THE RCSB ID CODE IS RCSB010911. | | |
| REMARK | 200 | | |
| REMARK | 200 EXPERIMENTAL DETAILS | | |
| REMARK | 200 EXPERIMENT TYPE | | : X-RAY DIFFRACTION |
| REMARK | 200 DATE OF DATA COLLECTION | | : 21-OCT-1999 |
| REMARK | 200 TEMPERATURE | (KELVIN) | : 100.0 |
| REMARK | 200 PH | | : 6.50 |
| REMARK | 200 NUMBER OF CRYSTALS USED | | : 1 |
| REMARK | 200 | | |
| REMARK | 200 SYNCHROTRON | (Y/N) | : Y |
| REMARK | 200 RADIATION SOURCE | | : NSLS |
| REMARK | 200 BEAMLINE | | : X4A |
| REMARK | 200 X-RAY GENERATOR MODEL | | : NULL |
| REMARK | 200 MONOCHROMATIC OR LAUE | (M/L) | : M |
| REMARK | 200 WAVELENGTH OR RANGE | (A) | : 0.9791 |
| REMARK | 200 MONOCHROMATOR | | : NULL |
| REMARK | 200 OPTICS | | : NULL |
| REMARK | 200 | | |
| REMARK | 200 DETECTOR TYPE | | : CCD |
| REMARK | 200 DETECTOR MANUFACTURER | | : ADSC QUANTUM 4 |
| REMARK | 200 INTENSITY-INTEGRATION SOFTWARE | | : DENZO |
| REMARK | 200 DATA SCALING SOFTWARE | | : SCALEPACK |
| REMARK | 200 | | |
| REMARK | 200 NUMBER OF UNIQUE REFLECTIONS | | : 47875 |
| REMARK | 200 RESOLUTION RANGE HIGH | (A) | : 1.600 |
| REMARK | 200 RESOLUTION RANGE LOW | (A) | : 25.000 |
| REMARK | 200 REJECTION CRITERIA | (SIGMA(I)) | : 0.000 |
| REMARK | 200 | | |
| REMARK | 200 OVERALL. | | |
| REMARK | 200 COMPLETENESS FOR RANGE | (%) | : 96.6 |
| REMARK | 200 DATA REDUNDANCY | | : 9.400 |
| REMARK | 200 R MERGE | (I) | : 0.04300 |
| REMARK | 200 R SYM | (I) | : NULL |

| | | |
|---|---|---|
| REMARK | 200 <I/SIGMA(I)> FOR THE DATA SET | : 16.4000 |
| REMARK | 200 | |
| REMARK | 200 IN THE HIGHEST RESOLUTION SHELL. | |

| | | |
|---|---|---|
| REMARK | 200 HIGHEST RESOLUTION SHELL, RANGE HIGH | (A) : 1.60 |
| REMARK | 200 HIGHEST RESOLUTION SHELL, RANGE LOW | (A) : 1.66 |

| | | | |
|---|---|---|---|
| REMARK | 200 COMPLETENESS FOR SHELL | (%) | : 86.0 |
| REMARK | 200 DATA REDUNDANCY IN SHELL | | : 4.30 |
| REMARK | 200 R MERGE FOR SHELL | (I) | 0.29000 |
| REMARK | 200 R SYM FOR SHELL | (I) | : NULL |
| REMARK | 200 <I/SIGMA(I)> FOR SHELL | | : NULL |
| REMARK | 200 | | |

| REMARK | 200 DIFFRACTION PROTOCOL: SINGLE WAVELENGTH | |
|---|---|---|
| REMARK | 200 METHOD USED TO DETERMINE THE STRUCTURE: NULL | |
| REMARK | 200 SOFTWARE USED: SHARP | |
| REMARK | 200 STARTING MODEL: NULL | |
| REMARK | 200 | |
| REMARK | 200 REMARK: NULL | |
| REMARK | 280 | |
| REMARK | 280 CRYSTAL | |
| REMARK | 280 SOLVENT CONTENT, VS | (%) : NULL |

| REMARK | 280 MATTHEWS COEFFICIENT, VM (ANGSTROMS**3/DA): NULL | | |
|---|---|---|---|
| REMARK | 280 | | |
| REMARK | 280 CRYSTALLIZATION CONDITIONS: 0.1M MES PH6.5, 10% W/V | | |
| REMARK | 280 POLYETHYLENE GLYCOL 20000, 3% W/V 1,6-HEXANDIOL | | |
| REMARK | 290 | | |
| REMARK | 290 CRYSTALLOGRAPHIC SYMMETRY | | |
| REMARK | 290 SYMMETRY OPERATORS FOR SPACE GROUP: P 21 21 2 | | |
| REMARK | 290 | | |
| REMARK | 290 | SYMOP | SYMMETRY |
| REMARK | 290 | NNNMMM | OPERATOR |
| REMARK | 290 | 1555 | X,Y,Z |
| REMARK | 290 | 2555 | −X,−Y,Z |
| REMARK | 290 | 3555 | 1/2−X,1/2+Y,−Z |
| REMARK | 290 | 4555 | 1/2+X,1/2−Y,−Z |
| REMARK | 290 | | |

| | | | |
|---|---|---|---|
| REMARK | 290 | WHERE NNN | −> OPERATOR NUMBER |
| REMARK | 290 | MMM | −> TRANSLATION VECTOR |
| REMARK | 290 | | |

| REMARK | 290 CRYSTALLOGRAPHIC SYMMETRY TRANSFORMATIONS | | | | | | |
|---|---|---|---|---|---|---|---|
| REMARK | 290 THE FOLLOWING TRANSFORMATIONS OPERATE ON THE ATOM/HETATM | | | | | | |
| REMARK | 290 RECORDS IN THIS ENTRY TO PRODUCE CRYSTALLOGRAPHICALLY | | | | | | |
| REMARK | 290 RELATED MOLECULES. | | | | | | |
| REMARK | 290 | SMTRY1 | 1 | 1.000000 | 0.000000 | 0.000000 | 0.00000 |
| REMARK | 290 | SMTRY2 | 1 | 0.000000 | 1.000000 | 0.000000 | 0.00000 |
| REMARK | 290 | SMTRY3 | 1 | 0.000000 | 0.000000 | 1.000000 | 0.00000 |
| REMARK | 290 | SMTRY1 | 2 | −1.000000 | 0.000000 | 0.000000 | 0.00000 |
| REMARK | 290 | SMTRY2 | 2 | 0.000000 | −1.000000 | 0.000000 | 0.00000 |
| REMARK | 290 | SMTRY3 | 2 | 0.000000 | 0.000000 | 1.000000 | 0.00000 |
| REMARK | 290 | SMTRY1 | 3 | −1.000000 | 0.000000 | 0.000000 | 62.89700 |
| REMARK | 290 | SMTRY2 | 3 | 0.000000 | 1.000000 | 0.000000 | 26.58350 |
| REMARK | 290 | SMTRY3 | 3 | 0.000000 | 0.000000 | −1.000000 | 0.00000 |
| REMARK | 290 | SMTRY1 | 4 | 1.000000 | 0.000000 | 0.000000 | 62.88700 |
| REMARK | 290 | SMTRY2 | 4 | 0.000000 | −1.000000 | 0.000000 | 26.58350 |
| REMARK | 290 | SMTRY3 | 4 | 0.000000 | 0.000000 | −1.000000 | 0.00000 |
| REMARK | 290 | | | | | | |
| REMARK | 290 REMARK: NULL | | | | | | |
| REMARK | 300 | | | | | | |
| REMARK | 300 BIOMOLECULE: 1 | | | | | | |
| REMARK | 300 THIS ENTRY CONTAINS THE CRYSTALLOGRAPHIC ASYMMETRIC UNIT | | | | | | |
| REMARK | 300 WHICH CONSISTS OF 2 CHAIN(S). SEE REMARK 35 C. FOR | | | | | | |
| REMARK | 300 INFORMATION ON GENERATING THE BIOLOGICAL MOLECULE(S). | | | | | | |
| REMARK | 350 | | | | | | |
| REMARK | 350 GENERATING THE BIOMOLECULE | | | | | | |
| REMARK | 350 COORDINATES FOR A COMPLETE MULTIMER REPRESENTING THE KNOWN | | | | | | |
| REMARK | 350 BIOLOGICALLY SIGNIFICANT OLIGOMERIZATION STATE OF THE | | | | | | |
| REMARK | 350 MOLECULE CAN BE GENERATED BY APPLYING BIOMT TRANSFORMATIONS | | | | | | |
| REMARK | 350 GIVEN BELOW. BOTH NON-CRYSTALLOGRAPHIC AND | | | | | | |
| REMARK | 350 CRYSTALLOGRAPHIC OPERATIONS ARE GIVEN. | | | | | | |
| REMARK | 350 | | | | | | |
| REMARK | 350 BIOMOLECULE: 1 | | | | | | |
| REMARK | 350 APPLY THE FOLLOWING TO CHAINS: A, B | | | | | | |
| REMARK | 350 | BIOMT1 | 1 | 1.000000 | 0.000000 | 0.000000 | 0.00000 |
| REMARK | 350 | BIOMT2 | 1 | 0.000000 | 1.000000 | 0.000000 | 0.00000 |
| REMARK | 350 | BIOMT3 | 1 | 0.000000 | 0.000000 | 1.000000 | 0.00000 |
| REMARK | 465 | | | | | | |

| REMARK | 465 MISSING RESIDUES | | | | | | | |
|---|---|---|---|---|---|---|---|---|
| REMARK | 465 THE FOLLOWING RESIDUES WERE NOT LOCATED IN THE | | | | | | | |
| REMARK | 465 EXPERIMENT. (M=MODEL NUMBER; RES=RESIDUE NAME; C=CHAIN | | | | | | | |
| REMARK | 465 IDENTIFIER; SSSEQ=SEQUENCE NUMBER; I=INSERTION CODE.) | | | | | | | |
| REMARK | 465 | | | | | | | |
| REMARK | 465 | M RES C SSSEQI | | | | | | |
| REMARK | 465 | GLU B 13 | | | | | | |
| REMARK | 465 | VAL B 14 | | | | | | |
| REMARK | 465 | LYS B 15 | | | | | | |
| REMARK | 465 | PRO B 16 | | | | | | |
| REMARK | 465 | GLU B 17 | | | | | | |
| REMARK | 465 | VAL B 18 | | | | | | |
| REMARK | 465 | LYS B 19 | | | | | | |
| REMARK | 470 | | | | | | | |
| REMARK | 470 MISSING ATOM | | | | | | | |
| REMARK | 470 THE FOLLOWING RESIDUES HAVE MISSING ATOMS (M=MODEL NUMBER; | | | | | | | |
| REMARK | 470 RES=RESIDUE NAME; C=CHAIN IDENTIFIER; SSEQ=SEQUENCE NUMBER; | | | | | | | |
| REMARK | 470 I=INSERTION CODE) : | | | | | | | |
| REMARK | 470 | M RES CSSEQI | | ATOMS | | | | |
| REMARK | 470 | PRO B | 20 | CB | CG | CD | | |
| REMARK | 470 | GLU B | 21 | CB | CG | CD | OE1 | 0E2 |
| REMARK | 470 | LYS B | 54 | CG | CD | CE | NZ | |
| REMARK | 470 | GLU B | 59 | CG | CD | OE1 | OE2 | |
| REMARK | 470 | ASP B | 75 | CB | CG | OD1 | OD2 | |
| REMARK | 470 | GLU B | 84 | CG | CD | OE1 | OE2 | |
| REMARK | 500 | | | | | | | |

| REMARK | 500 GEOMETRY AND STEREOCHEMISTRY | | | | | | | |
|---|---|---|---|---|---|---|---|---|
| REMARK | 500 SUBTOPIC: CLOSE CONTACTS IN SAME ASYMMETRIC UNIT | | | | | | | |
| REMARK | 500 | | | | | | | |
| REMARK | 500 THE FOLLOWING ATOMS ARE IN CLOSE CONTACT. | | | | | | | |
| REMARK | 500 | | | | | | | |
| REMARK | 500 | ATM1 | RES C | SSEQI | ATM2 | RES C | SSEQI | |
| REMARK | 500 | NE | ARG A | 422 | O | HOH | 377 | 1.12 |
| REMARK | 500 | CD | ARG A | 422 | O | HOH | 377 | 2.02 |
| REMARK | 500 | | | | O | HOH | 272 | |
| | | NE | ARG A | 422 | | | | 2.14 |
| REMARK | 500 | CZ | ARG A | 422 | O | HOH | 377 | 2.17 |
| REMARK | 500 | O | HOH | 457 | O | HOH | 718 | 2.17 |
| REMARK | 500 | | | | | | | |

| REMARK | 500 GEOMETRY AND STEREOCHEMISTRY | | | | | | | | |
|---|---|---|---|---|---|---|---|---|---|
| REMARK | 500 SUBTOPIC: CLOSE CONTACTS | | | | | | | | |
| REMARK | 500 | | | | | | | | |
| REMARK | 500 THE FOLLOWING ATOMS THAT ARE RELATED BY CRYSTALLOGRAPHIC | | | | | | | | |
| REMARK | 500 SYMMETRY ARE IN CLOSE CONTACT. AN ATOM LOCATED WITHIN 0.15 | | | | | | | | |
| REMARK | 500 ANGSTROMS OF A SYMMETRY RELATED ATOM IS ASSUMED TO BE ON A | | | | | | | | |
| REMARK | 500 SPECIAL POSITION AND IS, THEREFORE, LISTED IN REMARK 375 | | | | | | | | |
| REMARK | 500 INSTEAD OF REMARK 500. ATOMS WITH NON-BLANK ALTERNATE | | | | | | | | |
| REMARK | 500 LOCATION INDICATORS ARE NOT INCLUDED IN THE CALCULATIONS. | | | | | | | | |
| REMARK | 500 | | | | | | | | |
| REMARK | 500 DISTANCE CUTOFF: | | | | | | | | |
| REMARK | 500 2.2 ANGSTROMS FOR CONTACTS NOT INVOLVING HYDROGEN ATOMS | | | | | | | | |
| REMARK | 500 1.6 ANGSTROMS FOR CONTACTS INVOLVING HYDROGEN ATOMS | | | | | | | | |
| REMARK | 500 | | | | | | | | |
| REMARK | 500 | ATM1 | RES C | SSEQI | ATM2 | RES C | SSEQI | SSYMOP | DISTANCE |
| REMARK | 500 | O | HOH | 419 | O | HOH | 4 | 3558 | 1.28 |
| REMARK | 500 | O | HOH | 426 | O | HOH | 4 | 3558 | 1.62 |
| REMARK | 500 | O | HOH | 61 | O | HOH | 473 | 3558 | 1.68 |
| REMARK | 500 | O | HOH | 578 | O | HOH | 49 | 3548 | 2.07 |
| REMARK | 500 | O | HOH | 578 | O | HOH | 21 | 3548 | 2.18 |
| REMARK | 500 | O | HOH | 24 | O | HOH | 473 | 3558 | 2.18 |
| REMARK | 500 | | | | | | | | |

| | |
|---|---|
| REMARK | 500 GEOMETRY AND STEREOCHEMISTRY |
| REMARK | 500 SUBTOPIC: COVALENT BOND LENGTHS |
| REMARK | 500 |
| REMARK | 500 THE STEREOCHEMICAL PARAMETERS OF THE FOLLOWING RESIDUES |
| REMARK | 500 HAVE VALUES WHICH DEVIATE FROM EXPECTED VALUES BY MORE REMARK 500 THAN 6*RMSD |

(M=MODEL NUMBER; RES=RESIDUE NAME; C=CHAIN

| REMARK | 500 IDENTIFIER; SSEQ=SEQUENCE NUMBER; I=INSERTION CODE). | | | | | | |
|---|---|---|---|---|---|---|---|
| REMARK | 500 | | | | | | |
| REMARK | 500 STANDARD TABLE: | | | | | | |
| REMARK | 500 FORMAT: (10X,13,1X,2(A3,1X,A1,I4,A1,1X,A4,3X),F6.3) | | | | | | |
| REMARK | 500 | | | | | | |
| REMARK | 500 EXPECTED VALUES: ENGE AND HUBER, 1991 | | | | | | |
| REMARK | 500 | | | | | | |
| REMARK | 500 | M | RES CSSEQI | ATM1 | RES CSSEQI | ATM2 | DEVIATION |

| | | | | | | | | |
|---|---|---|---|---|---|---|---|---|
| REMARK | 500 | ARG A | 422 | CB | ARG A | 422 | CA | −0.335 |
| REMARK | 500 | GLY B | 98 | ^{O} | GLY B | 98 | C | 0.125 |

| REMARK | 500 | | | | | |
|---|---|---|---|---|---|---|
| REMARK | 500 GEOMETRY AND STEREOCHEMISTRY | | | | | |
| REMARK | 500 SUBTOPIC: COVALENT BOND ANGLES | | | | | |
| REMARK | 500 | | | | | |
| REMARK | 500 THE STEREOCHEMICAL PARAMETERS OF THE FOLLOWING RESIDUES | | | | | |
| REMARK | 500 HAVE VALUES WHICH DEVIATE FROM EXPECTED VALUES BY MORE | | | | | |
| REMARK | 500 THAN 6*RMSD (M=MODEL NUMBER; RES=RESIDUE NAME; C=CHAIN | | | | | |
| REMARK | 500 IDENTIFIER; SSEQ=SEQUENCE NUMBER; I=INSERTION CODE). | | | | | |
| REMARK | 500 | | | | | |
| REMARK | 500 STANDARD TABLE: | | | | | |
| REMARK | 500 FORMAT: (10X,I3,1X,A3,1X,A1,I4,A1,3(1X,A4,2X),12X,F5.1) | | | | | |
| REMARK | 500 | | | | | |
| REMARK | 500 EXPECTED VALUES: ENGH AND HUBER, 1991 | | | | | |
| REMARK | 500 | | | | | |
| REMARK | 500 | M | RES CSSEQI | ATM1 | ATM2 | ATM3 |

| | | | | | | | | | |
|---|---|---|---|---|---|---|---|---|---|
| REMARK | 500 | ARG A | 422 | CB | − | CA | − | C | ANGL. DEV. = 21.6 DEGREES |
| REMARK | 500 | GLU A | 423 | CB | − | CG | − | CD | ANGL. DEV. = 27.4 DEGREES |
| REMARK | 500 | | | | | | | | |

| REMARK | 500 GEOMETRY AND STEREOCHEMISTRY | | | | |
|---|---|---|---|---|---|
| REMARK | 500 SUBTOPIC: TORSION ANGLES | | | | |
| REMARK | 500 | | | | |
| REMARK | 500 TORSION ANGLES OUTSIDE THE EXPECTED RAMACHANDRAN REGIONS: | | | | |
| REMARK | 500 (M=MODEL NUMBER; RES=RESIDUE NAME; C=CHAIN IDENTIFIER; | | | | |
| REMARK | 500 SSEQ=SEQUENCE NUMBER; I=INSERTION CODE). | | | | |
| REMARK | 500 | | | | |
| REMARK | 500 STANDARD TABLE: | | | | |
| REMARK | 500 FORMAT: (10X,I3,1X,A3,1X,A1,I4,A1,4X, F7.2, 3X,F7.2) | | | | |
| REMARK | 500 | | | | |
| REMARK | 500 | M | RES CSSEQI | PSI | PHI |
| REMARK | 500 | | GLU A 423 | 112.62 | 151.65 |
| REMARK | 525 | | | | |

| REMARK | 525 SOLVENT | | | | |
|---|---|---|---|---|---|
| REMARK | 525 THE FOLLOWING SOLVENT MOLECULES LIE FARTHER THAN EXPECTED | | | | |
| REMARK | 525 FROM THE PROTEIN OR NUCLEIC ACID MOLECULE AND MAY BE | | | | |
| REMARK | 525 ASSOCIATED WITH A SYMMETRY RELATED MOLECULE (M=MODEL | | | | |
| REMARK | 525 NUMBER; RES=RESIDUE NAME; C=CHAIN IDENTIFIER; SSEQ=SEQUENCE | | | | |
| REMARK | 525 NUMBER; I=INSERTION CODE): | | | | |
| REMARK | 525 | | | | |
| REMARK | 525 | M | RES | CSSEQI | |
| REMARK | 525 | O | HOH | 322 | DISTANCE = 6.11 ANGSTROMS |
| REMARK | 525 | O | HOH | 420 | DISTANCE = 6.85 ANGSTROMS |
| REMARK | 525 | O | HOH | 457 | DISTANCE = 7.05 ANGSTROMS |
| REMARK | 525 | O | HOH | 544 | DISTANCE = 7.02 ANGSTROMS |
| REMARK | 525 | O | HOH | 627 | DISTANCE = 7.46 ANGSTROMS |
| REMARK | 525 | O | HOH | 673 | DISTANCE = 8.63 ANGSTROMS |
| REMARK | 525 | O | HOH | 718 | DISTANCE = 5.47 ANGSTROMS |
| REMARK | 525 | O | HOH | 746 | DISTANCE = 5.09 ANGSTROMS |
| REMARK | 999 | | | | |

| REMARK | 999 SEQUENCE | | | | | | | |
|---|---|---|---|---|---|---|---|---|
| REMARK | 999 RESIDUES 13-19 OF CHAIN B WERE NOT SEEN IN THE ELECTRON | | | | | | | |
| REMARK | 999 DENSITY. | | | | | | | |
| REMARK | 999 THE SIDE CHAINS OF RESIDUES 20, 21, 54, 59, 75, AND 84 | | | | | | | |
| REMARK | 999 IN CHAIN B WERE NOT SEEN IN THE ELECTRON DENSITY. | | | | | | | |
| DBREF | 1EUV A | 401 | 621 | GB | 1039457 | AAB68167 | 401 | 621 |
| DBREF | 1EUV B | 13 | 98 | SWS | Q12306 | SMT3_YEAST | 13 | 98 |

| | | | | | | |
|---|---|---|---|---|---|---|
| SEQADV | 1EUV GLY A | 401 | GB | 1039457 | LYS | 401 CLONING ARTIFACTS |
| SEQADV | 1EUV SER A | 402 | GB | 1039457 | LYS | 402 CLONING ARTIFACTS |

| | | | | |
|---|---|---|---|---|
| SEQRES | 1 | A | 221 | GLY SER LEU VAL PRO GLU LEU ASN GLU LYS ASP ASP ASP |
| SEQRES | 2 | A | 221 | GLN VAL GLN LYS ALA LEU ALA SER ARG GLU ASH THR GLN |
| SEQRES | 3 | A | 221 | LEU MET ASN ARG ASP ASN ILE GLU ILE THR VAL ARG ASP |
| SEQRES | 4 | A | 221 | PHE LYS THR LEU ALA PRO ARG ARG TRP LEU ASN ASP THR |
| SEQRES | 5 | A | 221 | ILE ILE GLU PHE PHE MET LYS TYR ILE GLU LYS SER THR |
| SEQRES | 6 | A | 221 | PRO ASN THR VAL ALA PHE ASN SER PHE PHE TYR THR ASN |
| SEQRES | 7 | A | 221 | LEU SER GLU ARG GLY TYR GLN GLY VAL ARG ARG TRP MET |
| SEQRES | 8 | A | 221 | LYS ARG LYS LYS THR GLN ILE ASP LYS LEU ASP LYS ILE |
| SEQRES | 9 | A | 221 | PHE THR PRO ILE ASN LEU ASN GLN SER HIS TRP ALA LEU |
| SEQRES | 10 | A | 221 | GLY ILE ILE ASP LEU LYS LYS LYS THR ILE GLY TYR VAL |
| SEQRES | 11 | A | 221 | ASP SER LEU SER ASN GLY PRO ASN ALA MET SER PHE ALA |
| SEQRES | 12 | A | 221 | ILE LEU THR ASP LEU GLN LYS TYR VAL MET GLU GLU SER |
| SEQRES | 13 | A | 221 | LYS HIS THR ILE GLY GLU ASP PHE ASP LEU ILE HIS LEU |
| SEQRES | 14 | A | 221 | ASP CYS PRO GLN GLN PRO ASN GLY TYR ASP CYS GLY ILE |
| SEQRES | 15 | A | 221 | TYR VAL CYS MET ASN THR LEU TYR GLY SER ALA ASP ALA |
| SEQRES | 16 | A | 221 | PRO LEU ASP PHE ASP TYR LYS ASP ALA ILE ARG MET ARG |
| SEQRES | 17 | A | 221 | ARG PHE ILE ALA HIS LEU ILE LEU THR ASP ALA LEU LYS |
| SEQRES | 1 | B | 86 | GLU VAL LYS PRO GLU VAL LYS PRO GLU THR HIS ILE ASN |
| SEQRES | 2 | B | 86 | LEU LYS VAL SER ASP GLY SER SER GLU ILE PHE PHE LYS |
| SEQRES | 3 | B | 86 | ILE LYS LYS THR THR PRO LEU ARG ARG LEU MET GLU ALA |
| SEQEES | 4 | B | 86 | PHE ALA LYS ARG GLN GLY LYS GLU MET ASP SER LEU ARG |
| SEQRES | 5 | B | 86 | PHE LEU TYR ASP GLY ILE ARG ILE GLN ALA ASP GLN THR |
| SEQRES | 6 | B | 86 | PRO GLU ASP LEU ASP MET GLU ASP ASN ASP ILE ILE GLU |
| SEQEES | 7 | B | 86 | ALA HIS ARG GLU GLN ILE GLY GLY |

| | | | |
|---|---|---|---|
| FORMUL | 3 | HOH | *432 (H2 O1) |

| | | | | | | | |
|---|---|---|---|---|---|---|---|
| HELIX | 1 | 1 ASN A | 408 | ALA A | 420 | 1 | 13 |
| HELIX | 2 | 2 VAL A | 437 | LYS A | 441 | 1 | 5 |
| HELIX | 3 | 3 THR A | 442 | ALA A | 444 | 5 | 3 |
| HELIX | 4 | 4 ASN A | 450 | THR A | 465 | 1 | 16 |
| HELIX | 5 | 5 SER A | 473 | GLY A | 483 | 1 | 11 |
| HELIX | 6 | 6 TYR A | 484 | VAL A | 487 | 5 | 4 |
| HELIX | 7 | 7 MET A | 491 | LYS A | 495 | 5 | 5 |
| HELIX | 8 | 8 GLN A | 497 | LEU A | 501 | 5 | 5 |
| HELIX | 9 | 9 ASN A | 538 | SER A | 556 | 1 | 19 |
| HELIX | 10 | 10 ASP A | 579 | ALA A | 583 | 1 | 15 |
| HELIX | 11 | 11 ASP A | 600 | THR A | 617 | 1 | 18 |
| HELIX | 12 | 12 LEU B | 45 | GLN B | 56 | 1 | 12 |
| HELIX | 13 | 13 GLU B | 59 | ASP B | 61 | 5 | 3 |

| SHEET | 1 | A 2 GLN A | 426 | ARG A | 430 | 0 | | | | | | |
|---|---|---|---|---|---|---|---|---|---|---|---|---|
| SHEET | 2 | A 2 ILE A | 433 | THR A | 436 | −1 | O | ILE A | 433 | N | ARG A | 430 |
| SHEET | 1 | B 5 THR A | 468 | ALA A | 470 | 0 | | | | | | |
| SHEET | 2 | B 5 LYS A | 503 | LEU A | 510 | 1 | O | LYS A | 503 | N | VAL A | 469 |
| SHEET | 3 | B 5 HIS A | 514 | ASP A | 521 | −1 | O | HIS A | 514 | N | LEU A | 510 |
| SHEET | 4 | B 5 THR A | 526 | VAL A | 530 | −1 | O | THR A | 526 | N | ASP A | 521 |
| SHEET | 5 | B 5 ASP A | 565 | LEU A | 569 | 1 | O | ASP A | 565 | N | ILE A | 527 |
| SHEET | 1 | C 5 GLU B | 34 | LYS B | 40 | 0 | | | | | | |
| SHEET | 2 | C 5 HIS B | 23 | SER B | 29 | −1 | N | ILE B | 24 | O | ILE B | 39 |
| SHEET | 3 | C 5 ASP B | 87 | ARG B | 93 | 1 | O | ASP B | 87 | N | LYS B | 27 |
| SHEET | 4 | C 5 LEU B | 63 | TYR B | 67 | −1 | N | ARG B | 64 | O | HIS B | 92 |
| SHEET | 5 | C 5 ILE B | 70 | ARG B | 71 | −1 | O | ILE B | 70 | N | TYR B | 67 |

| | | | | | |
|---|---|---|---|---|---|
| LIHK | SG CYS A | 580 | C | GLY B | 98 |

| | | | | | | | |
|---|---|---|---|---|---|---|---|
| CRYST1 | 125.774 | 53.167 | 54.262 | 90.00 | 90.00 | 90.00 P 21 21 2 | 4 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.007950 | 0.000000 | 0.000000 | 0.00000 |
| SCALE2 | 0.000000 | 0.018810 | 0.000000 | 0.00000 |
| SCALE3 | 0.000000 | 0.000000 | 0.018430 | 0.00000 |

| | | | | | | | | | | |
|---|---|---|---|---|---|---|---|---|---|---|
| ATOM | 1 | N | GLY A | 401 | 33.908 | 16.972 | 92.970 | 1.00 | 40.83 | N |

| | | | | | | | | | | | |
|---|---|---|---|---|---|---|---|---|---|---|---|
| ANISOU | 1 | N | GLY A | 401 | 5308 | 5152 | 5054 | 34 | −142 | 134 | N |

| | | | | | | | | | | |
|---|---|---|---|---|---|---|---|---|---|---|
| ATOM | 2 | CA | GLY A | 401 | 34.959 | 17.863 | 92.389 | 1.00 | 40.16 | C |

| | | | | | | | | | | | |
|---|---|---|---|---|---|---|---|---|---|---|---|
| ANISOU | 2 | CA | GLY A | 401 | 5199 | 5101 | 4959 | 86 | −131 | 93 | C |

| | | | | | | | | | | |
|---|---|---|---|---|---|---|---|---|---|---|
| ATOM | 3 | C | GLY A | 401 | 34.276 | 18.853 | 91.429 | 1.00 | 39.45 | C |

| | | | | | | | | | | | |
|---|---|---|---|---|---|---|---|---|---|---|---|
| ANISOU | 3 | C | GLY A | 401 | 5132 | 5117 | 4741 | 96 | −84 | 29 | C |

| | | | | | | | | | | |
|---|---|---|---|---|---|---|---|---|---|---|
| ATOM | 4 | O | GLY A | 401 | 33.765 | 19.898 | 91.836 | 1.00 | 41.56 | O |

| | | | | | | | | | | | |
|---|---|---|---|---|---|---|---|---|---|---|---|
| ANISOU | 4 | O | GLY A | 401 | 5289 | 5159 | 5342 | 113 | −1 | 8 | O |

| | | | | | | | | | | |
|---|---|---|---|---|---|---|---|---|---|---|
| ATOM | 5 | N | SER A | 402 | 34.308 | 18.564 | 90.144 | 1.00 | 36.00 | N |

| | | | | | | | | | | | |
|---|---|---|---|---|---|---|---|---|---|---|---|
| ANISOU | 5 | N | SER A | 402 | 4567 | 4596 | 4517 | 206 | −292 | 237 | N |

| | | | | | | | | | | |
|---|---|---|---|---|---|---|---|---|---|---|
| ATOM | 6 | CA | SER A | 402 | 33.593 | 19.410 | 89.176 | 1.00 | 31.44 | C |

| | | | | | | | | | | | |
|---|---|---|---|---|---|---|---|---|---|---|---|
| ANISOU | 6 | CA | SER A | 402 | 4023 | 3888 | 4035 | −16 | −105 | 48 | C |

| | | | | | | | | | | |
|---|---|---|---|---|---|---|---|---|---|---|
| ATOM | 7 | C | SER A | 402 | 33.490 | 18.568 | 87.917 | 1.00 | 28.88 | C |

| | | | | | | | | | | | |
|---|---|---|---|---|---|---|---|---|---|---|---|
| ANISOU | 7 | C | SER A | 402 | 3021 | 3603 | 3750 | 118 | −167 | 272 | C |

| | | | | | | | | | | |
|---|---|---|---|---|---|---|---|---|---|---|
| ATOM | 8 | O | SER A | 402 | 34.398 | 17.833 | 87.545 | 1.00 | 30.31 | O |

| | | | | | | | | | | | |
|---|---|---|---|---|---|---|---|---|---|---|---|
| ANISOU | 8 | O | SER A | 402 | 3857 | 3763 | 3896 | 281 | −148 | 218 | O |

| | | | | | | | | | | |
|---|---|---|---|---|---|---|---|---|---|---|
| ATOM | 9 | CB | SER A | 402 | 34.236 | 20.759 | 88.859 | 1.00 | 30.60 | C |

| | | | | | | | | | | | |
|---|---|---|---|---|---|---|---|---|---|---|---|
| ANISOU | 9 | CB | SER A | 402 | 3984 | 3744 | 3898 | 194 | −217 | 195 | C |

| | | | | | | | | | | |
|---|---|---|---|---|---|---|---|---|---|---|
| ATOM | 10 | OG | SER A | 402 | 33.428 | 21.458 | 87.901 | 1.00 | 28.46 | O |

| | | | | | | | | | | | |
|---|---|---|---|---|---|---|---|---|---|---|---|
| ANISOU | 10 | OG | SER A | 402 | 3763 | 3367 | 3684 | 85 | −148 | 70 | O |

| | | | | | | | | | | |
|---|---|---|---|---|---|---|---|---|---|---|
| ATOM | 11 | N | LEU A | 403 | 32.358 | 18.715 | 87.232 | 1.00 | 24.48 | N |

| | | | | | | | | | | | |
|---|---|---|---|---|---|---|---|---|---|---|---|
| ANISOU | 11 | N | LEU A | 403 | 3228 | 2729 | 3344 | 20 | −68 | 91 | N |

| | | | | | | | | | | |
|---|---|---|---|---|---|---|---|---|---|---|
| ATOM | 12 | CA | LEU A | 403 | 32.156 | 18.045 | 85.960 | 1.00 | 22.66 | C |

| | | | | | | | | | | | |
|---|---|---|---|---|---|---|---|---|---|---|---|
| ANISOU | 12 | CA | LEU A | 403 | 2907 | 2577 | 3127 | 65 | 11 | 223 | C |

| | | | | | | | | | | |
|---|---|---|---|---|---|---|---|---|---|---|
| ATOM | 13 | C | LEU A | 403 | 32.648 | 18.911 | 84.813 | 1.00 | 21.40 | C |

| | | | | | | | | | | | |
|---|---|---|---|---|---|---|---|---|---|---|---|
| ANISOU | 13 | C | LEU A | 403 | 2696 | 2438 | 2996 | 85 | −85 | 152 | C |

| | | | | | | | | | | |
|---|---|---|---|---|---|---|---|---|---|---|
| ATOM | 14 | O | LEU A | 403 | 32.533 | 18.491 | 83.671 | 1.00 | 21.43 | O |

| | | | | | | | | | | | |
|---|---|---|---|---|---|---|---|---|---|---|---|
| ANISOU | 14 | O | LEU A | 403 | 3033 | 2035 | 3075 | 125 | 44 | 179 | O |

| | | | | | | | | | | |
|---|---|---|---|---|---|---|---|---|---|---|
| ATOM | 15 | CB | LEU A | 403 | 30.654 | 17.761 | 85.763 | 1.00 | 21.94 | C |

| | | | | | | | | | | | |
|---|---|---|---|---|---|---|---|---|---|---|---|
| ANISOU | 15 | CB | LEU A | 403 | 2866 | 2470 | 3000 | 15 | 85 | 214 | C |

| | | | | | | | | | | |
|---|---|---|---|---|---|---|---|---|---|---|
| ATOM | 16 | CG | LEU A | 403 | 30.070 | 16.687 | 86.706 | 1.00 | 22.13 | C |

| | | | | | | | | | | | |
|---|---|---|---|---|---|---|---|---|---|---|---|
| ANISOU | 16 | CG | LEU A | 403 | 2787 | 2618 | 3003 | 68 | 171 | 266 | C |

| | | | | | | | | | | |
|---|---|---|---|---|---|---|---|---|---|---|
| ATOM | 17 | CD1 | LEU A | 403 | 28.607 | 16.437 | 86.413 | 1.00 | 23.95 | C |

| | | | | | | | | | | | |
|---|---|---|---|---|---|---|---|---|---|---|---|
| ANISOU | 17 | CD1 | LEU A | 403 | 3074 | 2778 | 3249 | −32 | −70 | 20 | C |

| | | | | | | | | | | |
|---|---|---|---|---|---|---|---|---|---|---|
| ATOM | 18 | CD2 | LEU A | 403 | 30.897 | 15.404 | 86.513 | 1.00 | 23.40 | C |

| | | | | | | | | | | | |
|---|---|---|---|---|---|---|---|---|---|---|---|
| ANISOU | 18 | CD2 | LEU A | 403 | 3198 | 2409 | 3283 | −36 | 69 | 60 | C |

| | | | | | | | | | | |
|---|---|---|---|---|---|---|---|---|---|---|
| ATOM | 19 | N | VAL A | 404 | 33.143 | 20.116 | 85.092 | 1.00 | 22.43 | N |

| | | | | | | | | | | | |
|---|---|---|---|---|---|---|---|---|---|---|---|
| ANISOU | 19 | N | VAL A | 404 | 2884 | 2486 | 3153 | 114 | −106 | 152 | N |

| | | | | | | | | | | |
|---|---|---|---|---|---|---|---|---|---|---|
| ATOM | 20 | CA | VAL A | 404 | 33.683 | 20.963 | 84.021 | 1.00 | 22.06 | C |

| | | | | | | | | | | | |
|---|---|---|---|---|---|---|---|---|---|---|---|
| ANISOU | 20 | CA | VAL A | 404 | 2880 | 2453 | 3050 | 80 | −22 | 91 | C |

| | | | | | | | | | | |
|---|---|---|---|---|---|---|---|---|---|---|
| ATOM | 21 | C | VAL A | 404 | 35.197 | 20.906 | 84.138 | 1.00 | 22.63 | C |

| | | | | | | | | | | | |
|---|---|---|---|---|---|---|---|---|---|---|---|
| ANISOU | 21 | C | VAL A | 404 | 3081 | 2515 | 3001 | 138 | −108 | 249 | C |

| | | | | | | | | | | |
|---|---|---|---|---|---|---|---|---|---|---|
| ATOM | 22 | O | VAL A | 404 | 35.785 | 21.433 | 85.067 | 1.00 | 24.07 | O |

| | | | | | | | | | | | |
|---|---|---|---|---|---|---|---|---|---|---|---|
| ANISOU | 22 | O | VAL A | 404 | 2846 | 3020 | 3279 | 128 | −221 | 178 | O |

| | | | | | | | | | | |
|---|---|---|---|---|---|---|---|---|---|---|
| ATOM | 23 | CB | VAL A | 404 | 33.147 | 22.416 | 84.244 | 1.00 | 21.98 | C |

| | | | | | | | | | | | |
|---|---|---|---|---|---|---|---|---|---|---|---|
| ANISOU | 23 | CB | VAL A | 404 | 2895 | 2505 | 2951 | 158 | −13 | 131 | C |

| | | | | | | | | | | |
|---|---|---|---|---|---|---|---|---|---|---|
| ATOM | 24 | CG1 | VAL A | 404 | 33.678 | 23.330 | 83.137 | 1.00 | 21.57 | C |

| | | | | | | | | | | | |
|---|---|---|---|---|---|---|---|---|---|---|---|
| ANISOU | 24 | CG1 | VAL A | 404 | 2633 | 2638 | 2926 | 59 | −25 | 132 | C |

| | | | | | | | | | | |
|---|---|---|---|---|---|---|---|---|---|---|
| ATOM | 25 | CG2 | VAL A | 404 | 31.636 | 22.469 | 84.333 | 1.00 | 20.47 | C |

| | | | | | | | | | | | |
|---|---|---|---|---|---|---|---|---|---|---|---|
| ANISOU | 25 | CG2 | VAL A | 404 | 2813 | 2171 | 2794 | −47 | −76 | 44 | C |

| | | | | | | | | | | |
|---|---|---|---|---|---|---|---|---|---|---|
| ATOM | 26 | N | PRO A | 405 | 35.860 | 20.317 | 83.172 | 1.00 | 23.66 | N |

| | | | | | | | | | | | |
|---|---|---|---|---|---|---|---|---|---|---|---|
| ANISOU | 26 | N | PRO A | 405 | 3008 | 2745 | 3235 | 89 | 31 | 168 | N |

| | | | | | | | | | | |
|---|---|---|---|---|---|---|---|---|---|---|
| ATOM | 27 | CA | PRO A | 405 | 37.313 | 20.171 | 83.266 | 1.00 | 26.77 | O |

| | | | | | | | | | | | |
|---|---|---|---|---|---|---|---|---|---|---|---|
| ANISOU | 27 | CA | PRO A | 405 | 3176 | 3461 | 3535 | 116 | −32 | 99 | C |

| | | | | | | | | | | |
|---|---|---|---|---|---|---|---|---|---|---|
| ATOM | 28 | C | PRO A | 405 | 38.073 | 21.419 | 82.867 | 1.00 | 30.16 | C |

| | | | | | | | | | | | |
|---|---|---|---|---|---|---|---|---|---|---|---|
| ANISOU | 28 | C | PRO A | 405 | 3742 | 3725 | 3992 | −99 | −6 | 186 | C |

| | | | | | | | | | | |
|---|---|---|---|---|---|---|---|---|---|---|
| ATOM | 29 | O | PRO A | 405 | 37.569 | 22.254 | 82.115 | 1.00 | 31.05 | O |

| | | | | | | | | | | | |
|---|---|---|---|---|---|---|---|---|---|---|---|
| ANISOU | 29 | O | PRO A | 405 | 3572 | 4012 | 4213 | 133 | −42 | 218 | O |

| | | | | | | | | | | |
|---|---|---|---|---|---|---|---|---|---|---|
| ATOM | 30 | CB | PRO A | 405 | 37.602 | 19.013 | 82.310 | 1.00 | 27.12 | C |

| | | | | | | | | | | | |
|---|---|---|---|---|---|---|---|---|---|---|---|
| ANISOU | 30 | CB | PRO A | 405 | 3320 | 3480 | 3506 | 35 | −40 | 78 | C |

| | | | | | | | | | | |
|---|---|---|---|---|---|---|---|---|---|---|
| ATOM | 31 | CG | PRO A | 405 | 36.575 | 19.165 | 81.253 | 1.00 | 26.08 | C |

| | | | | | | | | | | | |
|---|---|---|---|---|---|---|---|---|---|---|---|
| ANISOU | 31 | CG | PRO A | 405 | 3445 | 3222 | 3241 | 76 | 27 | 100 | C |

| | | | | | | | | | | |
|---|---|---|---|---|---|---|---|---|---|---|
| ATOM | 32 | CD | PRO A | 405 | 35.322 | 19.665 | 81.962 | 1.00 | 24.17 | C |

| | | | | | | | | | | | |
|---|---|---|---|---|---|---|---|---|---|---|---|
| ANISOU | 32 | CD | PRO A | 405 | 3120 | 2926 | 3135 | 87 | −29 | 248 | C |

| | | | | | | | | | | |
|---|---|---|---|---|---|---|---|---|---|---|
| ATOM | 33 | N | GLU A | 406 | 39.331 | 21.452 | 83.308 | 1.00 | 32.15 | N |

| | | | | | | | | | | | |
|---|---|---|---|---|---|---|---|---|---|---|---|
| ANISOU | 33 | N | GLU A | 406 | 3743 | 4267 | 4205 | 62 | −82 | 84 | N |

| | | | | | | | | | | |
|---|---|---|---|---|---|---|---|---|---|---|
| ATOM | 34 | CA | GLU A | 406 | 40.213 | 22.546 | 82.896 | 1.00 | 34.41 | C |

| | | | | | | | | | | | |
|---|---|---|---|---|---|---|---|---|---|---|---|
| ANISOU | 34 | CA | GLU A | 406 | 4328 | 4297 | 4449 | −26 | 60 | 111 | C |

| | | | | | | | | | | |
|---|---|---|---|---|---|---|---|---|---|---|
| ATOM | 35 | C | GLU A | 406 | 40.962 | 22.009 | 81.678 | 1.00 | 33.20 | C |

| | | | | | | | | | | | |
|---|---|---|---|---|---|---|---|---|---|---|---|
| ANISOU | 35 | C | GLU A | 406 | 4044 | 4240 | 4329 | −11 | −78 | 122 | C |

| | | | | | | | | | | |
|---|---|---|---|---|---|---|---|---|---|---|
| ATOM | 36 | O | GLU A | 406 | 41.302 | 20.813 | 81.564 | 1.00 | 34.60 | O |

| | | | | | | | | | | | |
|---|---|---|---|---|---|---|---|---|---|---|---|
| ANISOU | 36 | O | GLU A | 406 | 4388 | 4326 | 4431 | 23 | 75 | 21 | O |

| | | | | | | | | | | |
|---|---|---|---|---|---|---|---|---|---|---|
| ATOM | 37 | CD | GLU A | 406 | 41.151 | 22.996 | 84.009 | 1.00 | 38.62 | C |

| | | | | | | | | | | | |
|---|---|---|---|---|---|---|---|---|---|---|---|
| ANISOU | 37 | CB | GLU A | 406 | 4774 | 5087 | 4814 | 72 | −213 | −63 | C |

| | | | | | | | | | | |
|---|---|---|---|---|---|---|---|---|---|---|
| ATOM | 38 | CG | GLU A | 406 | 42.199 | 24.009 | 83.657 | 1.00 | 42.90 | C |

| | | | | | | | | | | | |
|---|---|---|---|---|---|---|---|---|---|---|---|
| ANISOU | 38 | CG | GLU A | 406 | 5265 | 5561 | 5476 | −144 | 28 | 90 | C |

| | | | | | | | | | | |
|---|---|---|---|---|---|---|---|---|---|---|
| ATOM | 39 | CD | GLU A | 406 | 42.099 | 25.492 | 83.825 | 1.00 | 46.53 | C |

| | | | | | | | | | | | |
|---|---|---|---|---|---|---|---|---|---|---|---|
| ANISOU | 39 | CD | GLU A | 406 | 5937 | 5757 | 5985 | 39 | 55 | −58 | C |

| | | | | | | | | | | |
|---|---|---|---|---|---|---|---|---|---|---|
| ATOM | 40 | OE1 | GLU A | 406 | 41.014 | 26.121 | 83.840 | 1.00 | 47.63 | O |

| | | | | | | | | | | | |
|---|---|---|---|---|---|---|---|---|---|---|---|
| ANISOU | 40 | OE1 | GLU A | 406 | 5807 | 6185 | 6105 | 64 | −66 | −3 | O |

| | | | | | | | | | | |
|---|---|---|---|---|---|---|---|---|---|---|
| ATOM | 41 | OE2 | GLU A | 406 | 43.201 | 26.138 | 83.932 | 1.00 | 48.05 | O |

| | | | | | | | | | | | |
|---|---|---|---|---|---|---|---|---|---|---|---|
| ANISOU | 41 | OE2 | GLU A | 406 | 5984 | 6029 | 6244 | −25 | −38 | −141 | O |

| | | | | | | | | | | |
|---|---|---|---|---|---|---|---|---|---|---|
| ATOM | 42 | N | LEU A | 407 | 41.204 | 22.874 | 90.717 | 1.00 | 30.75 | N |

| | | | | | | | | | | | |
|---|---|---|---|---|---|---|---|---|---|---|---|
| ANISOU | 42 | N | LEU A | 407 | 3597 | 4095 | 3993 | 67 | −165 | −26 | N |

| | | | | | | | | | | |
|---|---|---|---|---|---|---|---|---|---|---|
| ATOM | 43 | CA | LEU A | 407 | 41.903 | 22.531 | 79.517 | 1.00 | 30.07 | C |

| | | | | | | | | | | | |
|---|---|---|---|---|---|---|---|---|---|---|---|
| ANISOU | 43 | CA | LEU A | 407 | 3494 | 3990 | 3943 | 115 | −134 | 65 | C |

| | | | | | | | | | | |
|---|---|---|---|---|---|---|---|---|---|---|
| ATOM | 44 | C | LEU A | 407 | 43.435 | 22.550 | 79.725 | 1.00 | 31.27 | C |

| | | | | | | | | | | | |
|---|---|---|---|---|---|---|---|---|---|---|---|
| ANISOU | 44 | C | LEU A | 407 | 3497 | 4217 | 4169 | 2 | −112 | 20 | C |

| | | | | | | | | | | |
|---|---|---|---|---|---|---|---|---|---|---|
| ATOM | 45 | O | LEU A | 407 | 43.881 | 23.391 | 80.490 | 1.00 | 30.61 | O |

| | | | | | | | | | | | |
|---|---|---|---|---|---|---|---|---|---|---|---|
| ANISOU | 45 | O | LEU A | 407 | 3259 | 4132 | 4240 | 66 | −101 | −21 | O |

| | | | | | | | | | | |
|---|---|---|---|---|---|---|---|---|---|---|
| ATOM | 46 | CB | LEU A | 407 | 41.649 | 23.568 | 78.415 | 1.00 | 28.26 | C |

| | | | | | | | | | | | |
|---|---|---|---|---|---|---|---|---|---|---|---|
| ANISOU | 46 | CB | LEU A | 407 | 3228 | 3746 | 3763 | 29 | −113 | −33 | C |

| | | | | | | | | | | |
|---|---|---|---|---|---|---|---|---|---|---|
| ATOM | 47 | CG | LEU A | 407 | 40.187 | 23.605 | 77.902 | 1.00 | 26.89 | C |

| | | | | | | | | | | | |
|---|---|---|---|---|---|---|---|---|---|---|---|
| ANISOU | 47 | CG | LEU A | 407 | 3178 | 3570 | 3469 | 149 | −77 | 3 | C |

| | | | | | | | | | | |
|---|---|---|---|---|---|---|---|---|---|---|
| ATOM | 48 | CD1 | LEU A | 407 | 40.175 | 24.443 | 76.618 | 1.00 | 26.19 | C |

| | | | | | | | | | | | |
|---|---|---|---|---|---|---|---|---|---|---|---|
| ANISOU | 48 | CD1 | LEU A | 407 | 3100 | 3351 | 3499 | 61 | 37 | 19 | C |

| | | | | | | | | | | |
|---|---|---|---|---|---|---|---|---|---|---|
| ATOM | 49 | CD2 | LEU A | 407 | 39.686 | 22.202 | 77.667 | 1.00 | 26.73 | C |

| | | | | | | | | | | | |
|---|---|---|---|---|---|---|---|---|---|---|---|
| ANISOU | 49 | CD2 | LEU A | 407 | 2994 | 3575 | 3589 | 89 | −56 | 128 | C |

| | | | | | | | | | | |
|---|---|---|---|---|---|---|---|---|---|---|
| ATOM | 50 | N | ASN A | 408 | 44.132 | 21.677 | 79.008 | 1.00 | 33.58 | N |

| | | | | | | | | | | | |
|---|---|---|---|---|---|---|---|---|---|---|---|
| ANISOU | 50 | N | ASN A | 408 | 4138 | 4293 | 4328 | 106 | −18 | −20 | N |

| | | | | | | | | | | |
|---|---|---|---|---|---|---|---|---|---|---|
| ATOM | 51 | CA | ASN A | 408 | 45.598 | 21.797 | 79.140 | 1.00 | 35.58 | C |

| | | | | | | | | | | | |
|---|---|---|---|---|---|---|---|---|---|---|---|
| ANISOU | 51 | CA | ASN A | 408 | 4173 | 4655 | 4692 | 55 | −49 | −36 | C |

| | | | | | | | | | | |
|---|---|---|---|---|---|---|---|---|---|---|
| ATOM | 52 | C | ASN A | 408 | 46.002 | 23.026 | 78.339 | 1.00 | 36.95 | C |

| | | | | | | | | | | | |
|---|---|---|---|---|---|---|---|---|---|---|---|
| ANISOU | 52 | C | ASN A | 408 | 4461 | 4781 | 4796 | 35 | 1 | 52 | C |

| | | | | | | | | | | |
|---|---|---|---|---|---|---|---|---|---|---|
| ATOM | 53 | O | ASN A | 408 | 45.213 | 23.564 | 77.556 | 1.00 | 36.50 | O |

| | | | | | | | | | | | |
|---|---|---|---|---|---|---|---|---|---|---|---|
| ANISOU | 53 | O | ASN A | 408 | 4277 | 4887 | 4704 | −25 | 25 | 6 | O |

| | | | | | | | | | | |
|---|---|---|---|---|---|---|---|---|---|---|
| ATOM | 54 | CB | ASN A | 408 | 46.331 | 20.558 | 78.694 | 1.00 | 36.63 | C |

| | | | | | | | | | | | |
|---|---|---|---|---|---|---|---|---|---|---|---|
| ANISOU | 54 | CB | ASN A | 408 | 4492 | 4717 | 4708 | 200 | −6 | −31 | C |

| | | | | | | | | | | |
|---|---|---|---|---|---|---|---|---|---|---|
| ATOM | 55 | CG | ASN A | 408 | 46.048 | 20.146 | 77.271 | 1.00 | 36.32 | C |

| | | | | | | | | | | | |
|---|---|---|---|---|---|---|---|---|---|---|---|
| ANISOU | 55 | CG | ASN A | 408 | 4346 | 4754 | 4701 | 89 | 15 | −13 | C |

| | | | | | | | | | | |
|---|---|---|---|---|---|---|---|---|---|---|
| ATOM | 56 | OD1 | ASN A | 408 | 45.999 | 21.002 | 76.388 | 1.00 | 36.87 | O |

| | | | | | | | | | | | |
|---|---|---|---|---|---|---|---|---|---|---|---|
| ANISOU | 56 | OD1 | ASN A | 408 | 4442 | 4779 | 4790 | 159 | 114 | −1 | O |

| | | | | | | | | | | |
|---|---|---|---|---|---|---|---|---|---|---|
| ATOM | 57 | ND2 | ASN A | 408 | 45.870 | 18.847 | 77.051 | 1.00 | 37.25 | N |

| | | | | | | | | | | | |
|---|---|---|---|---|---|---|---|---|---|---|---|
| ANISOU | 57 | ND2 | ASN A | 408 | 4626 | 4785 | 4744 | 155 | 54 | −41 | N |

| | | | | | | | | | | |
|---|---|---|---|---|---|---|---|---|---|---|
| ATOM | 58 | N | GLU A | 409 | 47.255 | 23.461 | 78.456 | 1.00 | 38.07 | N |

| | | | | | | | | | | | |
|---|---|---|---|---|---|---|---|---|---|---|---|
| ANISOU | 58 | N | GLU A | 409 | 4528 | 4910 | 5025 | 46 | −64 | −21 | N |

| | | | | | | | | | | |
|---|---|---|---|---|---|---|---|---|---|---|
| ATOM | 59 | CA | GLU A | 409 | 47.684 | 24.665 | 77.761 | 1.00 | 39.20 | C |

| | | | | | | | | | | | |
|---|---|---|---|---|---|---|---|---|---|---|---|
| ANISOU | 59 | CA | GLU A | 409 | 4768 | 5065 | 5059 | −6 | 49 | 40 | C |

| | | | | | | | | | | |
|---|---|---|---|---|---|---|---|---|---|---|
| ATOM | 60 | C | GLU A | 409 | 47.541 | 24.565 | 76.260 | 1.00 | 37.19 | C |

| | | | | | | | | | | | |
|---|---|---|---|---|---|---|---|---|---|---|---|
| ANISOU | 60 | C | GLU A | 409 | 4244 | 4835 | 5052 | −106 | −66 | −31 | C |

| | | | | | | | | | | |
|---|---|---|---|---|---|---|---|---|---|---|
| ATOM | 61 | O | GLU A | 409 | 47.235 | 25.552 | 75.594 | 1.00 | 36.14 | O |

| | | | | | | | | | | | |
|---|---|---|---|---|---|---|---|---|---|---|---|
| ANISOU | 61 | O | GLU A | 409 | 3935 | 4839 | 4957 | −85 | −99 | −104 | O |

| | | | | | | | | | | |
|---|---|---|---|---|---|---|---|---|---|---|
| ATOM | 62 | CB | GLU A | 409 | 49.144 | 24.975 | 78.132 | 1.00 | 42.63 | C |

| | | | | | | | | | | | |
|---|---|---|---|---|---|---|---|---|---|---|---|
| ANISOU | 62 | CB | GLU A | 409 | 4977 | 5672 | 5550 | 27 | −114 | −47 | C |

| | | | | | | | | | | |
|---|---|---|---|---|---|---|---|---|---|---|
| ATOM | 63 | CG | GLU A | 409 | 49.702 | 26.229 | 77.505 | 1.00 | 46.82 | C |

| | | | | | | | | | | | |
|---|---|---|---|---|---|---|---|---|---|---|---|
| ANISOU | 63 | CG | GLU A | 409 | 5820 | 5939 | 6031 | −76 | 34 | 161 | C |

| | | | | | | | | | | |
|---|---|---|---|---|---|---|---|---|---|---|
| ATOM | 64 | CD | GLU A | 409 | 49.707 | 27.455 | 78.393 | 1.00 | 49.99 | C |

| | | | | | | | | | | | |
|---|---|---|---|---|---|---|---|---|---|---|---|
| ANISOU | 64 | CD | GLU A | 409 | 6365 | 6256 | 6375 | 1 | 19 | −79 | C |

| | | | | | | | | | | |
|---|---|---|---|---|---|---|---|---|---|---|
| ATOM | 65 | OE1 | GLU A | 409 | 49.064 | 27.396 | 79.471 | 1.00 | 52.03 | O |

| | | | | | | | | | | | |
|---|---|---|---|---|---|---|---|---|---|---|---|
| ANISOU | 65 | OE1 | GLU A | 409 | 6556 | 6681 | 6531 | −7 | 129 | 30 | O |

| | | | | | | | | | | |
|---|---|---|---|---|---|---|---|---|---|---|
| ATOM | 66 | OE2 | GLU A | 409 | 50.356 | 28.472 | 78.036 | 1.00 | 51.15 | O |

| | | | | | | | | | | | |
|---|---|---|---|---|---|---|---|---|---|---|---|
| ANISOU | 66 | OE2 | GLU A | 409 | 6452 | 6422 | 6560 | −59 | 78 | 69 | O |

| | | | | | | | | | | |
|---|---|---|---|---|---|---|---|---|---|---|
| ATOM | 67 | N | LYS A | 410 | 47.851 | 23.413 | 75.679 | 1.00 | 36.76 | N |

| | | | | | | | | | | | |
|---|---|---|---|---|---|---|---|---|---|---|---|
| ANISOU | 67 | N | LYS A | 410 | 4172 | 4828 | 4967 | 47 | −73 | 41 | N |

| | | | | | | | | | | |
|---|---|---|---|---|---|---|---|---|---|---|
| ATOM | 68 | CA | LYS A | 410 | 47.782 | 23.220 | 74.238 | 1.00 | 38.50 | C |

| | | | | | | | | | | | |
|---|---|---|---|---|---|---|---|---|---|---|---|
| ANISOU | 68 | CA | LYS A | 410 | 4491 | 5115 | 5022 | 47 | −84 | 8 | C |

| | | | | | | | | | | |
|---|---|---|---|---|---|---|---|---|---|---|
| ATOM | 69 | C | LYS A | 410 | 46.335 | 23.398 | 73.741 | 1.00 | 36.66 | C |

| | | | | | | | | | | | |
|---|---|---|---|---|---|---|---|---|---|---|---|
| ANISOU | 69 | C | LYS A | 410 | 4361 | 4727 | 4840 | 81 | 48 | 46 | C |

| | | | | | | | | | | |
|---|---|---|---|---|---|---|---|---|---|---|
| ATOM | 70 | O | LYS A | 410 | 46.059 | 24.076 | 72.753 | 1.00 | 35.34 | O |

| | | | | | | | | | | | |
|---|---|---|---|---|---|---|---|---|---|---|---|
| ANISOU | 70 | O | LYS A | 410 | 3997 | 4611 | 4820 | 113 | 2 | −50 | O |

| | | | | | | | | | | |
|---|---|---|---|---|---|---|---|---|---|---|
| ATOM | 71 | CB | LYS A | 410 | 48.361 | 21.841 | 73.927 | 1.00 | 41.63 | C |

| | | | | | | | | | | | |
|---|---|---|---|---|---|---|---|---|---|---|---|
| ANISOU | 71 | CB | LYS A | 410 | 5150 | 5213 | 5454 | 181 | 25 | 18 | C |

| | | | | | | | | | | |
|---|---|---|---|---|---|---|---|---|---|---|
| ATOM | 72 | CG | LYS A | 410 | 47.458 | 20.740 | 73.457 | 1.00 | 44.64 | C |

| | | | | | | | | | | | |
|---|---|---|---|---|---|---|---|---|---|---|---|
| ANISOU | 72 | CG | LYS A | 410 | 5565 | 5594 | 5803 | −20 | −68 | −91 | C |

| | | | | | | | | | | |
|---|---|---|---|---|---|---|---|---|---|---|
| ATOM | 73 | CD | LYS A | 410 | 47.498 | 19.428 | 74.240 | 1.00 | 48.31 | C |

| | | | | | | | | | | | |
|---|---|---|---|---|---|---|---|---|---|---|---|
| ANISOU | 73 | CD | LYS A | 410 | 5878 | 5778 | 5938 | 6 | 16 | 48 | C |

| | | | | | | | | | | |
|---|---|---|---|---|---|---|---|---|---|---|
| ATOM | 74 | CE | LYS A | 410 | 46.695 | 18.362 | 73.510 | 1.00 | 47.17 | C |

| | | | | | | | | | | | |
|---|---|---|---|---|---|---|---|---|---|---|---|
| ANISOU | 74 | CE | LYS A | 410 | 6037 | 5854 | 6030 | −11 | 2 | −47 | C |

| | | | | | | | | | | |
|---|---|---|---|---|---|---|---|---|---|---|
| ATOM | 75 | NZ | LYS A | 410 | 45.610 | 17.732 | 74.308 | 1.00 | 47.79 | N |

| | | | | | | | | | | | |
|---|---|---|---|---|---|---|---|---|---|---|---|
| ANISOU | 75 | NZ | LYS A | 410 | 6050 | 6032 | 6076 | 36 | 63 | −48 | N |

| | | | | | | | | | | |
|---|---|---|---|---|---|---|---|---|---|---|
| ATOM | 76 | N | ASP A | 411 | 45.423 | 22.744 | 74.439 | 1.00 | 35.18 | N |

| | | | | | | | | | | | |
|---|---|---|---|---|---|---|---|---|---|---|---|
| ANISOU | 76 | N | ASP A | 411 | 4135 | 4526 | 4707 | 148 | −61 | 18 | N |

| | | | | | | | | | | |
|---|---|---|---|---|---|---|---|---|---|---|
| ATOM | 77 | CA | ASP A | 411 | 43.998 | 22.806 | 74.077 | 1.00 | 32.85 | C |

| | | | | | | | | | | | |
|---|---|---|---|---|---|---|---|---|---|---|---|
| ANISOU | 77 | CA | ASP A | 411 | 3975 | 4136 | 4370 | 174 | 39 | 7 | C |

| | | | | | | | | | | |
|---|---|---|---|---|---|---|---|---|---|---|
| ATOM | 78 | C | ASP A | 411 | 43.466 | 24.227 | 74.250 | 1.00 | 32.37 | C |

| | | | | | | | | | | | |
|---|---|---|---|---|---|---|---|---|---|---|---|
| ANISOU | 78 | C | ASP A | 411 | 3909 | 4131 | 4259 | 142 | 3 | 46 | C |

| | | | | | | | | | | |
|---|---|---|---|---|---|---|---|---|---|---|
| ATOM | 79 | O | ASP A | 411 | 42.678 | 24.657 | 73.393 | 1.00 | 29.80 | O |

| | | | | | | | | | | | |
|---|---|---|---|---|---|---|---|---|---|---|---|
| ANISOU | 79 | O | ASP A | 411 | 3381 | 3767 | 4173 | 396 | 227 | −138 | O |

| | | | | | | | | | | |
|---|---|---|---|---|---|---|---|---|---|---|
| ATOM | 80 | CB | ASP A | 411 | 43.197 | 21.774 | 74.857 | 1.00 | 32.86 | C |

| | | | | | | | | | | | |
|---|---|---|---|---|---|---|---|---|---|---|---|
| ANISOU | 80 | CB | ASP A | 411 | 4022 | 4211 | 4254 | 108 | −15 | 22 | C |

| | | | | | | | | | | |
|---|---|---|---|---|---|---|---|---|---|---|
| ATOM | 81 | CG | ASP A | 411 | 43.488 | 20.337 | 74.494 | 1.00 | 33.23 | C |

| | | | | | | | | | | | |
|---|---|---|---|---|---|---|---|---|---|---|---|
| ANISOU | 81 | CG | ASP A | 411 | 4008 | 4275 | 4344 | 135 | −59 | 11 | C |

| | | | | | | | | | | |
|---|---|---|---|---|---|---|---|---|---|---|
| ATOM | 82 | OD1 | ASP A | 411 | 44.035 | 20.081 | 73.411 | 1.00 | 35.03 | O |

| | | | | | | | | | | | |
|---|---|---|---|---|---|---|---|---|---|---|---|
| ANISOU | 82 | OD1 | ASP A | 411 | 4439 | 4490 | 4382 | 246 | −69 | 26 | O |

| | | | | | | | | | | |
|---|---|---|---|---|---|---|---|---|---|---|
| ATOM | 83 | OD2 | ASP A | 411 | 43.189 | 19.384 | 75.244 | 1.00 | 33.96 | O |

| | | | | | | | | | | | |
|---|---|---|---|---|---|---|---|---|---|---|---|
| ANISOU | 83 | OD2 | ASP A | 411 | 4179 | 4148 | 4576 | 132 | −130 | −3 | O |

| | | | | | | | | | | |
|---|---|---|---|---|---|---|---|---|---|---|
| ATOM | 84 | N | ASP A | 412 | 43.863 | 24.920 | 75.299 | 1.00 | 32.19 | N |

| | | | | | | | | | | | |
|---|---|---|---|---|---|---|---|---|---|---|---|
| ANISOU | 84 | N | ASP A | 412 | 3713 | 4205 | 4314 | 87 | 46 | 33 | N |

| | | | | | | | | | | |
|---|---|---|---|---|---|---|---|---|---|---|
| ATOM | 85 | CA | ASP A | 412 | 43.434 | 26.311 | 75.507 | 1.00 | 33.69 | C |

| | | | | | | | | | | | |
|---|---|---|---|---|---|---|---|---|---|---|---|
| ANISOU | 85 | CA | ASP A | 412 | 3959 | 4210 | 4631 | 40 | 98 | 88 | C |

| | | | | | | | | | | |
|---|---|---|---|---|---|---|---|---|---|---|
| ATOM | 86 | C | ASP A | 412 | 43.948 | 27.194 | 74.377 | 1.00 | 33.14 | C |

| | | | | | | | | | | | |
|---|---|---|---|---|---|---|---|---|---|---|---|
| ANISOU | 86 | C | ASP A | 412 | 3915 | 4285 | 4392 | 36 | 52 | 9 | C |

| | | | | | | | | | | |
|---|---|---|---|---|---|---|---|---|---|---|
| ATOM | 87 | O | ASP A | 412 | 43.258 | 28.083 | 73.893 | 1.00 | 32.19 | O |

| | | | | | | | | | | | |
|---|---|---|---|---|---|---|---|---|---|---|---|
| ANISOU | 87 | O | ASP A | 412 | 3706 | 4220 | 4306 | −124 | 86 | 76 | O |

| | | | | | | | | | | |
|---|---|---|---|---|---|---|---|---|---|---|
| ATOM | 88 | CB | ASP A | 412 | 43.889 | 26.811 | 76.859 | 1.00 | 37.61 | C |

| | | | | | | | | | | | |
|---|---|---|---|---|---|---|---|---|---|---|---|
| ANISOU | 88 | CB | ASP A | 412 | 4614 | 4918 | 4758 | −20 | −31 | 11 | C |

| | | | | | | | | | | |
|---|---|---|---|---|---|---|---|---|---|---|
| ATOM | 89 | CG | ASP A | 412 | 43.634 | 28.229 | 77.299 | 1.00 | 42.22 | C |

| | | | | | | | | | | | |
|---|---|---|---|---|---|---|---|---|---|---|---|
| ANISOU | 89 | CG | ASP A | 412 | 5493 | 5127 | 5420 | 76 | −11 | −79 | C |

| | | | | | | | | | | |
|---|---|---|---|---|---|---|---|---|---|---|
| ATOM | 90 | OD1 | ASP A | 412 | 43.240 | 29.189 | 76.599 | 1.00 | 41.69 | O |

| | | | | | | | | | | | |
|---|---|---|---|---|---|---|---|---|---|---|---|
| ANISOU | 90 | OD1 | ASP A | 412 | 5302 | 5222 | 5316 | 61 | −25 | −61 | O |

| | | | | | | | | | | |
|---|---|---|---|---|---|---|---|---|---|---|
| ATOM | 91 | OD2 | ASP A | 412 | 43.693 | 28.409 | 78.555 | 1.00 | 45.86 | O |

| | | | | | | | | | | | |
|---|---|---|---|---|---|---|---|---|---|---|---|
| ANISOU | 91 | OD2 | ASP A | 412 | 6083 | 5826 | 5515 | 31 | 37 | −123 | O |

| | | | | | | | | | | |
|---|---|---|---|---|---|---|---|---|---|---|
| ATOM | 92 | N | ASP A | 413 | 45.186 | 26.985 | 73.919 | 1.00 | 33.23 | N |

| | | | | | | | | | | | |
|---|---|---|---|---|---|---|---|---|---|---|---|
| ANISOU | 92 | N | ASP A | 413 | 3947 | 4297 | 4380 | 21 | 128 | −36 | N |

| | | | | | | | | | | |
|---|---|---|---|---|---|---|---|---|---|---|
| ATOM | 93 | CA | ASP A | 413 | 45.726 | 27.835 | 72.859 | 1.00 | 33.18 | C |

| | | | | | | | | | | | |
|---|---|---|---|---|---|---|---|---|---|---|---|
| ANISOU | 93 | CA | ASP A | 413 | 3965 | 4321 | 4322 | 33 | 143 | −40 | C |

| | | | | | | | | | | |
|---|---|---|---|---|---|---|---|---|---|---|
| ATOM | 94 | C | ASP A | 413 | 44.980 | 27.675 | 71.552 | 1.00 | 31.01 | C |

| | | | | | | | | | | | |
|---|---|---|---|---|---|---|---|---|---|---|---|
| ANISOU | 94 | C | ASP A | 413 | 3436 | 4091 | 4253 | 101 | 290 | 1 | C |

| | | | | | | | | | | |
|---|---|---|---|---|---|---|---|---|---|---|
| ATOM | 95 | O | ASP A | 413 | 44.794 | 28.636 | 70.810 | 1.00 | 29.88 | O |

| | | | | | | | | | | | |
|---|---|---|---|---|---|---|---|---|---|---|---|
| ANISOU | 95 | O | ASP A | 413 | 3073 | 4219 | 4060 | 21 | 307 | 40 | O |

| | | | | | | | | | | |
|---|---|---|---|---|---|---|---|---|---|---|
| ATOM | 96 | CB | ASP A | 413 | 47.209 | 27.472 | 72.638 | 1.00 | 36.39 | C |

| | | | | | | | | | | | |
|---|---|---|---|---|---|---|---|---|---|---|---|
| ANISOU | 96 | CB | ASP A | 413 | 4115 | 4850 | 4860 | 115 | 172 | −14 | C |

| | | | | | | | | | | |
|---|---|---|---|---|---|---|---|---|---|---|
| ATOM | 97 | CG | ASP A | 413 | 48.100 | 28.137 | 73.665 | 1.00 | 39.77 | C |

| | | | | | | | | | | | |
|---|---|---|---|---|---|---|---|---|---|---|---|
| ANISOU | 97 | CG | ASP A | 413 | 4814 | 5190 | 5107 | −49 | −46 | −75 | C |

| | | | | | | | | | | |
|---|---|---|---|---|---|---|---|---|---|---|
| ATOM | 98 | OD1 | ASP A | 413 | 47.634 | 29.045 | 74.401 | 1.00 | 41.39 | O |

| | | | | | | | | | | | |
|---|---|---|---|---|---|---|---|---|---|---|---|
| ANISOU | 98 | OD1 | ASP A | 413 | 4961 | 5376 | 5390 | 84 | 25 | −129 | O |

| | | | | | | | | | | |
|---|---|---|---|---|---|---|---|---|---|---|
| ATOM | 99 | OD2 | ASP A | 413 | 49.289 | 27.737 | 73.710 | 1.00 | 40.98 | O |

| | | | | | | | | | | | |
|---|---|---|---|---|---|---|---|---|---|---|---|
| ANISOU | 99 | OD2 | ASP A | 413 | 4919 | 5314 | 5337 | 34 | −4 | −36 | O |

| | | | | | | | | | | |
|---|---|---|---|---|---|---|---|---|---|---|
| ATOM | 100 | N | GLN A | 414 | 44.505 | 26.453 | 71.311 | 1.00 | 30.31 | N |

| | | | | | | | | | | | |
|---|---|---|---|---|---|---|---|---|---|---|---|
| ANISOU | 100 | N | GLN A | 414 | 3359 | 4105 | 4050 | 75 | 261 | −112 | N |

| | | | | | | | | | | |
|---|---|---|---|---|---|---|---|---|---|---|
| ATOM | 101 | CA | GLN A | 414 | 43.725 | 26.129 | 70.124 | 1.00 | 30.58 | C |

| | | | | | | | | | | | |
|---|---|---|---|---|---|---|---|---|---|---|---|
| ANISOU | 101 | CA | GLN A | 414 | 3461 | 4162 | 3996 | 104 | 183 | 27 | C |

| | | | | | | | | | | |
|---|---|---|---|---|---|---|---|---|---|---|
| ATOM | 102 | C | GLN A | 414 | 42.429 | 26.940 | 70.167 | 1.00 | 28.35 | C |

| | | | | | | | | | | | |
|---|---|---|---|---|---|---|---|---|---|---|---|
| ANISOU | 102 | C | GLN A | 414 | 3389 | 3698 | 3685 | −51 | 97 | −26 | C |

| | | | | | | | | | | |
|---|---|---|---|---|---|---|---|---|---|---|
| ATOM | 103 | O | GLN A | 414 | 42.049 | 27.542 | 69.168 | 1.00 | 27.07 | O |

| | | | | | | | | | | | |
|---|---|---|---|---|---|---|---|---|---|---|---|
| ANISOU | 103 | O | GLN A | 414 | 2863 | 3826 | 3597 | 16 | 297 | −56 | O |

| | | | | | | | | | | |
|---|---|---|---|---|---|---|---|---|---|---|
| ATOM | 104 | CB | GLN A | 414 | 43.437 | 24.628 | 70.097 | 1.00 | 33.29 | C |

| | | | | | | | | | | | |
|---|---|---|---|---|---|---|---|---|---|---|---|
| ANISOU | 104 | CB | GLN A | 414 | 3960 | 4231 | 4459 | 68 | 140 | −46 | C |

| | | | | | | | | | | |
|---|---|---|---|---|---|---|---|---|---|---|
| ATOM | 105 | CG | GLN A | 414 | 42.767 | 24.142 | 68.831 | 1.00 | 36.44 | C |

| | | | | | | | | | | | |
|---|---|---|---|---|---|---|---|---|---|---|---|
| ANISOU | 105 | CG | GLN A | 414 | 4523 | 4811 | 4510 | 57 | 2 | −86 | C |

| | | | | | | | | | | |
|---|---|---|---|---|---|---|---|---|---|---|
| ATOM | 106 | CD | GLN A | 414 | 42.332 | 22.694 | 68.807 | 1.00 | 38.97 | C |

| | | | | | | | | | | | |
|---|---|---|---|---|---|---|---|---|---|---|---|
| ANISOU | 106 | CD | GLN A | 414 | 4948 | 4869 | 4990 | 37 | 17 | −30 | C |

| | | | | | | | | | | |
|---|---|---|---|---|---|---|---|---|---|---|
| ATOM | 107 | OE1 | GLN A | 414 | 42.176 | 21.994 | 69.813 | 1.00 | 39.33 | O |

| | | | | | | | | | | | |
|---|---|---|---|---|---|---|---|---|---|---|---|
| ANISOU | 107 | OE1 | GLN A | 414 | 4907 | 5059 | 4978 | 145 | 130 | −10 | O |

| | | | | | | | | | | |
|---|---|---|---|---|---|---|---|---|---|---|
| ATOM | 108 | NE2 | GLN A | 414 | 42.089 | 22.204 | 67.581 | 1.00 | 40.14 | N |

| | | | | | | | | | | | |
|---|---|---|---|---|---|---|---|---|---|---|---|
| ANISOU | 108 | NE2 | GLN A | 414 | 5108 | 5146 | 4995 | 97 | 38 | −99 | N |

| | | | | | | | | | | |
|---|---|---|---|---|---|---|---|---|---|---|
| ATOM | 109 | N | VAL A | 415 | 41.776 | 26.933 | 71.336 | 1.00 | 27.93 | N |

| | | | | | | | | | | | |
|---|---|---|---|---|---|---|---|---|---|---|---|
| ANISOU | 109 | N | VAL A | 415 | 3254 | 3688 | 3669 | 24 | 120 | −105 | N |

| | | | | | | | | | | |
|---|---|---|---|---|---|---|---|---|---|---|
| ATOM | 110 | CA | VAL A | 415 | 40.577 | 27.777 | 71.508 | 1.00 | 27.64 | C |

| | | | | | | | | | | | |
|---|---|---|---|---|---|---|---|---|---|---|---|
| ANISOU | 110 | CA | VAL A | 415 | 3407 | 3565 | 3531 | 33 | 157 | 56 | C |

| | | | | | | | | | | |
|---|---|---|---|---|---|---|---|---|---|---|
| ATOM | 111 | C | VAL A | 415 | 40.894 | 29.248 | 71.236 | 1.00 | 28.30 | C |

| | | | | | | | | | | | |
|---|---|---|---|---|---|---|---|---|---|---|---|
| ANISOU | 111 | C | VAL A | 415 | 3498 | 3560 | 3696 | 50 | 97 | −32 | C |

| | | | | | | | | | | |
|---|---|---|---|---|---|---|---|---|---|---|
| ATOM | 112 | O | VAL A | 415 | 40.207 | 29.882 | 70.438 | 1.00 | 26.80 | O |

| | | | | | | | | | | | |
|---|---|---|---|---|---|---|---|---|---|---|---|
| ANISOU | 112 | O | VAL A | 415 | 3141 | 3682 | 3361 | −139 | 318 | 40 | O |

| | | | | | | | | | | |
|---|---|---|---|---|---|---|---|---|---|---|
| ATOM | 113 | CB | VAL A | 415 | 40.021 | 27.623 | 72.938 | 1.00 | 26.21 | C |

| | | | | | | | | | | | |
|---|---|---|---|---|---|---|---|---|---|---|---|
| ANISOU | 113 | CB | VAL A | 415 | 3199 | 3290 | 3470 | 81 | 169 | −84 | C |

| | | | | | | | | | | |
|---|---|---|---|---|---|---|---|---|---|---|
| ATOM | 114 | CG1 | VAL A | 415 | 38.926 | 28.661 | 73.191 | 1.00 | 24.26 | C |

| | | | | | | | | | | | |
|---|---|---|---|---|---|---|---|---|---|---|---|
| ANISOU | 114 | CG1 | VAL A | 415 | 3073 | 3127 | 3018 | 55 | 100 | −31 | C |

| | | | | | | | | | | |
|---|---|---|---|---|---|---|---|---|---|---|
| ATOM | 115 | CG2 | VAL A | 415 | 39.500 | 26.210 | 73.170 | 1.00 | 26.40 | C |

| | | | | | | | | | | | |
|---|---|---|---|---|---|---|---|---|---|---|---|
| ANISOU | 115 | CG2 | VAL A | 415 | 3219 | 3299 | 3513 | 87 | 187 | −135 | C |

| | | | | | | | | | | |
|---|---|---|---|---|---|---|---|---|---|---|
| ATOM | 116 | N | GLN A | 416 | 41.937 | 29.807 | 71.834 | 1.00 | 30.21 | N |

| | | | | | | | | | | | |
|---|---|---|---|---|---|---|---|---|---|---|---|
| ANISOU | 116 | N | GLN A | 416 | 3553 | 3958 | 3968 | −40 | 20 | −38 | N |

| | | | | | | | | | | |
|---|---|---|---|---|---|---|---|---|---|---|
| ATOM | 117 | CA | GLN A | 416 | 42.305 | 31.223 | 71.647 | 1.00 | 32.66 | C |

| | | | | | | | | | | | |
|---|---|---|---|---|---|---|---|---|---|---|---|
| ANISOU | 117 | CA | GLN A | 416 | 3944 | 4116 | 4350 | −84 | 87 | 5 | C |

| | | | | | | | | | | |
|---|---|---|---|---|---|---|---|---|---|---|
| ATOM | 118 | C | GLN A | 416 | 42.582 | 31.547 | 70.189 | 1.00 | 32.92 | C |

| | | | | | | | | | | | |
|---|---|---|---|---|---|---|---|---|---|---|---|
| ANTSOU | 118 | C | GLN A | 416 | 3868 | 4255 | 4385 | −173 | 37 | 64 | C |

| | | | | | | | | | | |
|---|---|---|---|---|---|---|---|---|---|---|
| ATOM | 119 | O | GLN A | 416 | 42.093 | 32.561 | 69.677 | 1.00 | 32.32 | O |

| | | | | | | | | | | | |
|---|---|---|---|---|---|---|---|---|---|---|---|
| ANISOU | 119 | O | GLN A | 416 | 3600 | 4158 | 4521 | −304 | 242 | 144 | O |

| | | | | | | | | | | |
|---|---|---|---|---|---|---|---|---|---|---|
| ATOM | 120 | CB | GLN A | 416 | 43.441 | 31.586 | 72.610 | 1.00 | 34.16 | C |

| | | | | | | | | | | | |
|---|---|---|---|---|---|---|---|---|---|---|---|
| ANISOU | 120 | CB | GLN A | 416 | 4088 | 4399 | 4493 | −71 | 6 | −42 | C |

| | | | | | | | | | | |
|---|---|---|---|---|---|---|---|---|---|---|
| ATOM | 121 | CG | GLN A | 416 | 43.730 | 33.065 | 72.837 | 1.00 | 35.92 | C |

| | | | | | | | | | | | |
|---|---|---|---|---|---|---|---|---|---|---|---|
| ANISOU | 121 | CG | GLN A | 416 | 4471 | 4456 | 4721 | −68 | 78 | −51 | C |

| | | | | | | | | | | |
|---|---|---|---|---|---|---|---|---|---|---|
| ATOM | 122 | CD | GLN A | 416 | 42.815 | 33.729 | 73.852 | 1.00 | 36.77 | C |

| | | | | | | | | | | | |
|---|---|---|---|---|---|---|---|---|---|---|---|
| ANISON | 122 | CD | GLN A | 416 | 4614 | 4558 | 4798 | −51 | 112 | −100 | C |

| | | | | | | | | | | |
|---|---|---|---|---|---|---|---|---|---|---|
| ATOM | 123 | OE1 | GLN A | 416 | 41.740 | 33.219 | 74.178 | 1.00 | 35.20 | O |

| | | | | | | | | | | | |
|---|---|---|---|---|---|---|---|---|---|---|---|
| ANISOU | 123 | OE1 | GLN A | 416 | 4475 | 4258 | 4641 | −6 | 99 | −75 | O |

| | | | | | | | | | | |
|---|---|---|---|---|---|---|---|---|---|---|
| ATOM | 124 | NE2 | GLN A | 416 | 43.257 | 34.888 | 74.359 | 1.00 | 36.70 | N |

| | | | | | | | | | | | |
|---|---|---|---|---|---|---|---|---|---|---|---|
| ANISOU | 124 | NE2 | GLN A | 416 | 4667 | 4464 | 4813 | −11 | 3 | 1 | N |

| | | | | | | | | | | |
|---|---|---|---|---|---|---|---|---|---|---|
| ATOM | 125 | N | LYS A | 417 | 43.251 | 30.665 | 69.434 | 1.00 | 34.04 | N |

| | | | | | | | | | | | |
|---|---|---|---|---|---|---|---|---|---|---|---|
| ANISOU | 125 | N | LYS A | 417 | 4038 | 4303 | 4592 | −131 | 97 | −37 | N |

| | | | | | | | | | | |
|---|---|---|---|---|---|---|---|---|---|---|
| ATOM | 126 | CA | LYS A | 417 | 43.497 | 30.890 | 68.012 | 1.00 | 35.76 | C |

| | | | | | | | | | | | |
|---|---|---|---|---|---|---|---|---|---|---|---|
| ANISOU | 126 | CA | LYS A | 417 | 4375 | 4586 | 4628 | −163 | 11 | 63 | C |

| | | | | | | | | | | |
|---|---|---|---|---|---|---|---|---|---|---|
| ATOM | 127 | C | LYS A | 417 | 42.222 | 30.925 | 67.179 | 1.00 | 34.58 | C |

| | | | | | | | | | | | |
|---|---|---|---|---|---|---|---|---|---|---|---|
| ANISOU | 127 | C | LYS A | 417 | 4260 | 4343 | 4536 | −93 | 100 | 111 | C |

| | | | | | | | | | | |
|---|---|---|---|---|---|---|---|---|---|---|
| ATOM | 128 | O | LYS A | 417 | 42.025 | 31.750 | 66.289 | 1.00 | 34.18 | O |

| | | | | | | | | | | | |
|---|---|---|---|---|---|---|---|---|---|---|---|
| ANISOU | 128 | O | LYS A | 417 | 4068 | 4441 | 4476 | −196 | 163 | 181 | O |

| | | | | | | | | | | |
|---|---|---|---|---|---|---|---|---|---|---|
| ATOM | 129 | CE | LYS A | 417 | 44.337 | 29.768 | 67.395 | 1.00 | 38.84 | C |

| | | | | | | | | | | | |
|---|---|---|---|---|---|---|---|---|---|---|---|
| ANISOU | 129 | CB | LYS A | 417 | 4878 | 4805 | 5074 | −25 | 150 | −58 | C |

| | | | | | | | | | | |
|---|---|---|---|---|---|---|---|---|---|---|
| ATOM | 130 | CG | LYS A | 417 | 45.608 | 29.400 | 68.112 | 1.00 | 42.20 | C |

| | | | | | | | | | | | |
|---|---|---|---|---|---|---|---|---|---|---|---|
| ANISOU | 130 | CG | LYS A | 417 | 5220 | 5398 | 5416 | 10 | −105 | 56 | C |

| | | | | | | | | | | |
|---|---|---|---|---|---|---|---|---|---|---|
| ATOM | 131 | CD | LYS A | 417 | 46.384 | 28.344 | 67.308 | 1.00 | 44.53 | C |

| | | | | | | | | | | | |
|---|---|---|---|---|---|---|---|---|---|---|---|
| ANISOU | 131 | CD | LYS A | 417 | 5611 | 5583 | 5725 | 80 | 66 | −21 | C |

| | | | | | | | | | | |
|---|---|---|---|---|---|---|---|---|---|---|
| ATOM | 132 | CE | LYS A | 417 | 47.848 | 28.366 | 67.760 | 1.00 | 46.02 | C |

| | | | | | | | | | | | |
|---|---|---|---|---|---|---|---|---|---|---|---|
| ANISOU | 132 | CE | LYS A | 417 | 5687 | 5874 | 5924 | 25 | −24 | 35 | C |

| | | | | | | | | | | |
|---|---|---|---|---|---|---|---|---|---|---|
| ATOM | 133 | NZ | LYS A | 417 | 48.448 | 29.714 | 67.513 | 1.00 | 46.73 | N |

| | | | | | | | | | | | |
|---|---|---|---|---|---|---|---|---|---|---|---|
| ANISOU | 133 | NZ | LYS A | 417 | 5835 | 5884 | 6035 | 5 | 2 | −4 | N |

| | | | | | | | | | | |
|---|---|---|---|---|---|---|---|---|---|---|
| ATOM | 134 | N | ALA A | 418 | 41.342 | 29.957 | 67.464 | 1.00 | 33.25 | N |

| | | | | | | | | | | | |
|---|---|---|---|---|---|---|---|---|---|---|---|
| ANISOU | 134 | N | ALA A | 418 | 4054 | 4272 | 4309 | −34 | 52 | 71 | N |

| | | | | | | | | | | |
|---|---|---|---|---|---|---|---|---|---|---|
| ATOM | 135 | CA | ALA A | 418 | 40.061 | 29.907 | 66.748 | 1.00 | 31.26 | C |

| | | | | | | | | | | | |
|---|---|---|---|---|---|---|---|---|---|---|---|
| ANISOU | 135 | CA | ALA A | 418 | 3863 | 4041 | 3973 | −119 | 175 | 18 | C |

| | | | | | | | | | | |
|---|---|---|---|---|---|---|---|---|---|---|
| ATOM | 136 | C | ALA A | 418 | 39.239 | 31.154 | 67.014 | 1.00 | 30.31 | C |

| | | | | | | | | | | | |
|---|---|---|---|---|---|---|---|---|---|---|---|
| ANISOU | 136 | C | ALA A | 418 | 3761 | 3858 | 3898 | −223 | 55 | 116 | C |

| | | | | | | | | | | |
|---|---|---|---|---|---|---|---|---|---|---|
| ATOM | 137 | O | ALA A | 418 | 38.595 | 31.695 | 66.130 | 1.00 | 28.84 | O |

| | | | | | | | | | | | |
|---|---|---|---|---|---|---|---|---|---|---|---|
| ANISOU | 137 | O | ALA A | 418 | 3437 | 3784 | 3737 | −282 | 277 | 98 | O |

| | | | | | | | | | | |
|---|---|---|---|---|---|---|---|---|---|---|
| ATOM | 138 | CB | ALA A | 418 | 39.321 | 28.633 | 67.148 | 1.00 | 30.90 | C |

| | | | | | | | | | | | |
|---|---|---|---|---|---|---|---|---|---|---|---|
| ANISOU | 138 | CB | ALA A | 418 | 3821 | 3956 | 3965 | −83 | 79 | 14 | C |

| | | | | | | | | | | |
|---|---|---|---|---|---|---|---|---|---|---|
| ATOM | 139 | N | LEU A | 419 | 39.237 | 31.719 | 68.222 | 1.00 | 30.33 | N |

| | | | | | | | | | | | |
|---|---|---|---|---|---|---|---|---|---|---|---|
| ANISOU | 139 | N | LEU A | 419 | 3656 | 3838 | 4029 | −309 | 186 | 10 | N |

| | | | | | | | | | | |
|---|---|---|---|---|---|---|---|---|---|---|
| ATOM | 140 | CA | LEU A | 419 | 38.502 | 32.928 | 68.551 | 1.00 | 32.41 | C |

| | | | | | | | | | | | |
|---|---|---|---|---|---|---|---|---|---|---|---|
| ANISOU | 140 | CA | LEU A | 419 | 3996 | 4102 | 4216 | −64 | 20 | −35 | C |

| | | | | | | | | | | |
|---|---|---|---|---|---|---|---|---|---|---|
| ATOM | 141 | C | LEU A | 419 | 39.056 | 34.157 | 67.827 | 1.00 | 35.75 | C |

| | | | | | | | | | | | |
|---|---|---|---|---|---|---|---|---|---|---|---|
| ANISOU | 141 | C | LEU A | 419 | 4660 | 4318 | 4604 | −119 | 15 | 148 | C |

| | | | | | | | | | | |
|---|---|---|---|---|---|---|---|---|---|---|
| ATOM | 142 | O | LEU A | 419 | 38.334 | 35.129 | 67.632 | 1.00 | 36.41 | O |

| | | | | | | | | | | | |
|---|---|---|---|---|---|---|---|---|---|---|---|
| ANISOU | 142 | O | LEU A | 419 | 4665 | 4311 | 4857 | −76 | 125 | 39 | O |

| | | | | | | | | | | |
|---|---|---|---|---|---|---|---|---|---|---|
| ATOM | 143 | CB | LEU A | 419 | 38.553 | 33.202 | 70.049 | 1.00 | 31.38 | C |

| | | | | | | | | | | | |
|---|---|---|---|---|---|---|---|---|---|---|---|
| ANISOU | 143 | CB | LEU A | 419 | 3959 | 3825 | 4141 | −96 | 9 | 8 | C |

| | | | | | | | | | | |
|---|---|---|---|---|---|---|---|---|---|---|
| ATOM | 144 | CG | LEU A | 419 | 37.792 | 32.275 | 70.985 | 1.00 | 29.76 | C |

| | | | | | | | | | | | |
|---|---|---|---|---|---|---|---|---|---|---|---|
| ANISOU | 144 | CG | LEU A | 419 | 3581 | 3926 | 3801 | −17 | −12 | −50 | C |

| | | | | | | | | | | |
|---|---|---|---|---|---|---|---|---|---|---|
| ATOM | 145 | CD1 | LEU A | 419 | 38.175 | 32.531 | 72.435 | 1.00 | 29.11 | C |

| | | | | | | | | | | | |
|---|---|---|---|---|---|---|---|---|---|---|---|
| ANISOU | 145 | CD1 | LEU A | 419 | 3735 | 3547 | 3780 | −9 | −39 | −54 | C |

| | | | | | | | | | | |
|---|---|---|---|---|---|---|---|---|---|---|
| ATOM | 146 | CD2 | LEU A | 419 | 36.278 | 32.458 | 70.802 | 1.00 | 28.85 | C |

| | | | | | | | | | | | |
|---|---|---|---|---|---|---|---|---|---|---|---|
| ANISOU | 146 | CD2 | LEU A | 419 | 3560 | 3738 | 3664 | 16 | −41 | −129 | C |

| | | | | | | | | | | |
|---|---|---|---|---|---|---|---|---|---|---|
| ATOM | 147 | N | ALA A | 420 | 40.310 | 34.071 | 67.420 | 1.00 | 38.82 | N |

| | | | | | | | | | | | |
|---|---|---|---|---|---|---|---|---|---|---|---|
| ANISOU | 147 | N | ALA A | 420 | 4696 | 4998 | 5057 | 20 | 22 | −23 | N |

| | | | | | | | | | | |
|---|---|---|---|---|---|---|---|---|---|---|
| ATOM | 148 | CA | ALA A | 420 | 40.925 | 35.169 | 66.695 | 1.00 | 42.84 | C |

| | | | | | | | | | | | |
|---|---|---|---|---|---|---|---|---|---|---|---|
| ANISOU | 148 | CA | ALA A | 420 | 5345 | 5283 | 5649 | −74 | 94 | 198 | C |

| | | | | | | | | | | |
|---|---|---|---|---|---|---|---|---|---|---|
| ATOM | 149 | C | ALA A | 420 | 40.876 | 34.939 | 65.197 | 1.00 | 46.54 | C |

| | | | | | | | | | | | |
|---|---|---|---|---|---|---|---|---|---|---|---|
| ANISOU | 149 | C | ALA A | 420 | 5897 | 5976 | 5809 | 72 | 59 | 70 | C |

| | | | | | | | | | | |
|---|---|---|---|---|---|---|---|---|---|---|
| ATOM | 150 | O | ALA A | 420 | 41.473 | 35.806 | 64.528 | 1.00 | 46.77 | O |

| | | | | | | | | | | | |
|---|---|---|---|---|---|---|---|---|---|---|---|
| ANISOU | 150 | O | ALA A | 420 | 5836 | 5990 | 5944 | −58 | 37 | 24 | O |

| | | | | | | | | | | |
|---|---|---|---|---|---|---|---|---|---|---|
| ATOM | 151 | CB | ALA A | 420 | 42.383 | 35.300 | 67.133 | 1.00 | 42.55 | C |

| | | | | | | | | | | | |
|---|---|---|---|---|---|---|---|---|---|---|---|
| ANISOU | 151 | CB | ALA A | 420 | 5357 | 5328 | 5484 | −26 | 68 | 112 | C |

| | | | | | | | | | | |
|---|---|---|---|---|---|---|---|---|---|---|
| ATOM | 152 | N | SER A | 421 | 40.238 | 33.887 | 64.653 | 1.00 | 49.87 | N |

| | | | | | | | | | | | |
|---|---|---|---|---|---|---|---|---|---|---|---|
| ANISOU | 152 | N | SER A | 421 | 6154 | 6195 | 6599 | −86 | −39 | −4 | N |

| | | | | | | | | | | |
|---|---|---|---|---|---|---|---|---|---|---|
| ATOM | 153 | CA | SER A | 421 | 40.428 | 33.802 | 63.210 | 1.00 | 54.60 | C |

| | | | | | | | | | | | |
|---|---|---|---|---|---|---|---|---|---|---|---|
| ANISOU | 153 | CA | SER A | 421 | 6935 | 7048 | 6764 | −44 | 46 | −15 | C |

| | | | | | | | | | | |
|---|---|---|---|---|---|---|---|---|---|---|
| ATOM | 154 | C | SER A | 421 | 39.687 | 34.907 | 62.460 | 1.00 | 57.01 | C |

| | | | | | | | | | | | |
|---|---|---|---|---|---|---|---|---|---|---|---|
| ANISOU | 154 | C | SER A | 421 | 7152 | 7227 | 7282 | 115 | −91 | 7 | C |

| | | | | | | | | | | |
|---|---|---|---|---|---|---|---|---|---|---|
| ATOM | 155 | O | SER A | 421 | 38.600 | 35.374 | 62.710 | 1.00 | 56.19 | O |

| | | | | | | | | | | | |
|---|---|---|---|---|---|---|---|---|---|---|---|
| ANISOU | 155 | O | SER A | 421 | 7102 | 7171 | 7076 | −31 | −51 | −7 | O |

| | | | | | | | | | | |
|---|---|---|---|---|---|---|---|---|---|---|
| ATOM | 156 | CB | SER A | 421 | 40.370 | 32.496 | 62.468 | 1.00 | 56.08 | C |

| | | | | | | | | | | | |
|---|---|---|---|---|---|---|---|---|---|---|---|
| ANISOU | 156 | CB | SER A | 421 | 7128 | 7019 | 7159 | −29 | 39 | −43 | C |

| | | | | | | | | | | |
|---|---|---|---|---|---|---|---|---|---|---|
| ATOM | 157 | OG | SER A | 421 | 39.715 | 32.602 | 61.206 | 1.00 | 57.68 | O |

| | | | | | | | | | | | |
|---|---|---|---|---|---|---|---|---|---|---|---|
| ANISOU | 157 | OG | SER A | 421 | 7242 | 7382 | 7291 | −26 | −32 | 29 | O |

| | | | | | | | | | | |
|---|---|---|---|---|---|---|---|---|---|---|
| ATOM | 158 | N | ARG A | 422 | 40.514 | 35.340 | 61.508 | 1.00 | 60.66 | N |

| | | | | | | | | | | | |
|---|---|---|---|---|---|---|---|---|---|---|---|
| ANISOU | 158 | N | ARG A | 422 | 7599 | 7808 | 7640 | −44 | 96 | −22 | N |

| | | | | | | | | | | |
|---|---|---|---|---|---|---|---|---|---|---|
| ATOM | 159 | CA | ARG A | 422 | 40.244 | 36.346 | 60.509 | 1.00 | 63.35 | C |

| | | | | | | | | | | | |
|---|---|---|---|---|---|---|---|---|---|---|---|
| ANISOU | 159 | CA | ARG A | 422 | 8092 | 7994 | 7986 | 82 | 32 | 148 | C |

| | | | | | | | | | | |
|---|---|---|---|---|---|---|---|---|---|---|
| ATOM | 160 | C | ARG A | 422 | 39.395 | 35.667 | 59.422 | 1.00 | 65.36 | C |

| | | | | | | | | | | | |
|---|---|---|---|---|---|---|---|---|---|---|---|
| ANISOU | 160 | C | ARG A | 422 | 8264 | 8317 | 8251 | −5 | −44 | 27 | C |

| | | | | | | | | | | |
|---|---|---|---|---|---|---|---|---|---|---|
| ATOM | 161 | O | ARG A | 422 | 39.766 | 35.074 | 58.416 | 1.00 | 66.33 | O |

| | | | | | | | | | | | |
|---|---|---|---|---|---|---|---|---|---|---|---|
| ANISOU | 161 | O | ARG A | 422 | 8454 | 8401 | 8346 | 39 | 22 | −11 | O |

| | | | | | | | | | | |
|---|---|---|---|---|---|---|---|---|---|---|
| ATOM | 162 | CB | ARG A | 422 | 40.997 | 37.273 | 60.466 | 0.00 | 59.92 | C |
| ATOM | 163 | CG | ARG A | 422 | 40.723 | 38.706 | 60.039 | 0.00 | 56.99 | C |
| ATOM | 164 | CD | ARG A | 422 | 40.341 | 39.575 | 61.227 | 0.00 | 54.62 | C |
| ATOM | 165 | NE | ARG A | 422 | 40.086 | 40.956 | 60.834 | 0.00 | 53.35 | N |
| ATOM | 166 | CZ | ARG A | 422 | 39.736 | 41.924 | 61.676 | 0.00 | 51.98 | C |
| ATOM | 167 | NH1 | ARG A | 422 | 39.600 | 41.659 | 62.970 | 0.00 | 52.27 | N |
| ATOM | 168 | NH2 | ARG A | 422 | 39.524 | 43.153 | 61.226 | 0.00 | 52.14 | N |
| ATOM | 169 | N | GLU A | 423 | 38.106 | 35.761 | 59.692 | 1.00 | 65.38 | N |

| | | | | | | | | | | | |
|---|---|---|---|---|---|---|---|---|---|---|---|
| ANISOU | 169 | N | GLU A | 423 | 8268 | 8334 | 8238 | −4 | 9 | 42 | N |

| | | | | | | | | | | |
|---|---|---|---|---|---|---|---|---|---|---|
| ATOM | 170 | CA | GLU A | 423 | 36.966 | 35.458 | 58.893 | 1.00 | 65.22 | C |

| | | | | | | | | | | | |
|---|---|---|---|---|---|---|---|---|---|---|---|
| ANISOU | 170 | CA | GLU A | 423 | 8230 | 8200 | 8349 | −55 | 16 | 23 | C |

| | | | | | | | | | | |
|---|---|---|---|---|---|---|---|---|---|---|
| ATOM | 171 | C | GLU A | 423 | 36.092 | 34.307 | 59.292 | 1.00 | 63.05 | C |

| | | | | | | | | | | | |
|---|---|---|---|---|---|---|---|---|---|---|---|
| ANISOU | 171 | C | GLU A | 423 | 7921 | 8063 | 7972 | 66 | −33 | −5 | C |

| | | | | | | | | | | |
|---|---|---|---|---|---|---|---|---|---|---|
| ATOM | 172 | O | GLU A | 423 | 36.424 | 33.404 | 60.046 | 1.00 | 62.56 | O |

| | | | | | | | | | | | |
|---|---|---|---|---|---|---|---|---|---|---|---|
| ANISOU | 172 | O | GLU A | 423 | 7907 | 7979 | 7885 | −22 | 1 | −22 | O |

| | | | | | | | | | | |
|---|---|---|---|---|---|---|---|---|---|---|
| ATOM | 173 | CB | GLU A | 423 | 37.262 | 35.696 | 57.389 | 1.00 | 67.48 | C |

| | | | | | | | | | | | |
|---|---|---|---|---|---|---|---|---|---|---|---|
| ANISOU | 173 | CB | GLU A | 423 | 8545 | 8674 | 8420 | −14 | 34 | 21 | C |

| | | | | | | | | | | |
|---|---|---|---|---|---|---|---|---|---|---|
| ATOM | 174 | CG | GLU A | 423 | 37.198 | 37.195 | 57.260 | 1.00 | 69.65 | C |

| | | | | | | | | | | | |
|---|---|---|---|---|---|---|---|---|---|---|---|
| ANISOU | 174 | CG | GLU A | 423 | 8868 | 8736 | 8862 | −4 | −1 | −25 | C |

| | | | | | | | | | | |
|---|---|---|---|---|---|---|---|---|---|---|
| ATOM | 175 | CD | GLU A | 423 | 37.431 | 38.247 | 56.269 | 1.00 | 70.95 | C |

| | | | | | | | | | | | |
|---|---|---|---|---|---|---|---|---|---|---|---|
| ANISOU | 175 | CD | GLU A | 423 | 9060 | 8927 | 8969 | −18 | 36 | 61 | C |

| | | | | | | | | | | |
|---|---|---|---|---|---|---|---|---|---|---|
| ATOM | 176 | OE1 | GLU A | 423 | 36.914 | 38.204 | 55.128 | 1.00 | 71.70 | O |

| | | | | | | | | | | | |
|---|---|---|---|---|---|---|---|---|---|---|---|
| ANISOU | 176 | OE1 | GLU A | 423 | 9103 | 9102 | 9039 | −12 | −18 | 6 | O |

| | | | | | | | | | | |
|---|---|---|---|---|---|---|---|---|---|---|
| ATOM | 177 | OE2 | GLU A | 423 | 38.135 | 39.235 | 56.635 | 1.00 | 71.54 | O |

| | | | | | | | | | | | |
|---|---|---|---|---|---|---|---|---|---|---|---|
| ANISOU | 177 | OE2 | GLU A | 423 | 9115 | 9008 | 9058 | −42 | 5 | −11 | O |

| | | | | | | | | | | |
|---|---|---|---|---|---|---|---|---|---|---|
| ATOM | 178 | N | ASN A | 424 | 34.827 | 34.492 | 58.891 | 1.00 | 60.44 | N |

| | | | | | | | | | | | |
|---|---|---|---|---|---|---|---|---|---|---|---|
| ANISOU | 178 | N | ASN A | 424 | 7734 | 7603 | 7627 | −93 | 107 | 30 | N |

| | | | | | | | | | | |
|---|---|---|---|---|---|---|---|---|---|---|
| ATOM | 179 | CA | ASN A | 424 | 33.724 | 33.638 | 59.273 | 1.00 | 57.76 | C |

| | | | | | | | | | | | |
|---|---|---|---|---|---|---|---|---|---|---|---|
| ANISOU | 179 | CA | ASN A | 424 | 7432 | 7276 | 7238 | 44 | 2 | −65 | C |

| | | | | | | | | | | |
|---|---|---|---|---|---|---|---|---|---|---|
| ATOM | 180 | C | ASN A | 424 | 33.628 | 32.341 | 58.492 | 1.00 | 55.12 | C |

| | | | | | | | | | | | |
|---|---|---|---|---|---|---|---|---|---|---|---|
| ANISOU | 180 | C | ASN A | 424 | 6949 | 7060 | 6936 | 41 | 52 | 111 | C |

| | | | | | | | | | | |
|---|---|---|---|---|---|---|---|---|---|---|
| ATOM | 181 | O | ASN A | 424 | 32.775 | 32.089 | 57.660 | 1.00 | 55.76 | O |

| | | | | | | | | | | | |
|---|---|---|---|---|---|---|---|---|---|---|---|
| ANISOU | 181 | O | ASN A | 424 | 7065 | 7155 | 6965 | −61 | 44 | 6 | O |

| | | | | | | | | | | |
|---|---|---|---|---|---|---|---|---|---|---|
| ATOM | 182 | CB | ASN A | 424 | 32.402 | 34.424 | 59.250 | 1.00 | 57.84 | C |

| | | | | | | | | | | | |
|---|---|---|---|---|---|---|---|---|---|---|---|
| ANISOU | 182 | CB | ASN A | 424 | 7352 | 7310 | 7313 | −13 | 37 | 17 | C |

| | | | | | | | | | | |
|---|---|---|---|---|---|---|---|---|---|---|
| ATOM | 183 | CG | ASN A | 424 | 31.332 | 33.693 | 60.060 | 1.00 | 57.45 | C |

| | | | | | | | | | | | |
|---|---|---|---|---|---|---|---|---|---|---|---|
| ANISOU | 183 | CG | ASN A | 424 | 7353 | 7259 | 7216 | 5 | 34 | −2 | C |

| | | | | | | | | | | |
|---|---|---|---|---|---|---|---|---|---|---|
| ATOM | 184 | OE1 | ASN A | 424 | 30.127 | 33.860 | 59.837 | 1.00 | 57.50 | O |

| | | | | | | | | | | | |
|---|---|---|---|---|---|---|---|---|---|---|---|
| ANISOU | 184 | OE1 | ASN A | 424 | 7350 | 7270 | 7227 | 25 | 72 | 0 | O |

| | | | | | | | | | | |
|---|---|---|---|---|---|---|---|---|---|---|
| ATOM | 185 | ND2 | ASN A | 424 | 31.807 | 32.870 | 60.983 | 1.00 | 56.42 | N |

| | | | | | | | | | | | |
|---|---|---|---|---|---|---|---|---|---|---|---|
| ANISOU | 185 | ND2 | ASN A | 424 | 7196 | 7128 | 7113 | −30 | 63 | −45 | N |

| | | | | | | | | | | |
|---|---|---|---|---|---|---|---|---|---|---|
| ATOM | 186 | N | THR A | 425 | 34.530 | 31.452 | 58.852 | 1.00 | 51.97 | N |

| | | | | | | | | | | | |
|---|---|---|---|---|---|---|---|---|---|---|---|
| ANISOU | 186 | N | THR A | 425 | 6741 | 6514 | 6491 | −139 | 151 | −38 | N |

| | | | | | | | | | | |
|---|---|---|---|---|---|---|---|---|---|---|
| ATOM | 187 | CA | THR A | 425 | 34.702 | 30.138 | 58.285 | 1.00 | 49.63 | C |

| | | | | | | | | | | | |
|---|---|---|---|---|---|---|---|---|---|---|---|
| ANISOU | 187 | CA | THR A | 425 | 6342 | 6305 | 6208 | −147 | 138 | 134 | C |

| | | | | | | | | | | |
|---|---|---|---|---|---|---|---|---|---|---|
| ATOM | 188 | C | THR A | 425 | 33.915 | 29.109 | 59.092 | 1.00 | 45.80 | C |

| | | | | | | | | | | | |
|---|---|---|---|---|---|---|---|---|---|---|---|
| ANISOU | 188 | C | THR A | 425 | 5959 | 5706 | 5738 | −10 | −1 | −132 | C |

| | | | | | | | | | | |
|---|---|---|---|---|---|---|---|---|---|---|
| ATOM | 189 | O | THR A | 425 | 33.537 | 29.348 | 60.235 | 1.00 | 43.73 | O |

| | | | | | | | | | | | |
|---|---|---|---|---|---|---|---|---|---|---|---|
| ANISOU | 189 | O | THR A | 425 | 5602 | 5307 | 5705 | −99 | 100 | 1 | O |

| | | | | | | | | | | |
|---|---|---|---|---|---|---|---|---|---|---|
| ATOM | 190 | CB | THR A | 425 | 36.205 | 29.790 | 58.329 | 1.00 | 51.23 | C |

| | | | | | | | | | | | |
|---|---|---|---|---|---|---|---|---|---|---|---|
| ANISOU | 190 | CB | THR A | 425 | 6461 | 6520 | 6486 | 2 | 46 | 16 | C |

| | | | | | | | | | | |
|---|---|---|---|---|---|---|---|---|---|---|
| ATOM | 191 | OG1 | THR A | 425 | 36.604 | 29.503 | 59.677 | 1.00 | 52.07 | O |

| | | | | | | | | | | | |
|---|---|---|---|---|---|---|---|---|---|---|---|
| ANISOU | 191 | OG1 | THR A | 425 | 6610 | 6666 | 6509 | 10 | 42 | 32 | O |

| | | | | | | | | | | |
|---|---|---|---|---|---|---|---|---|---|---|
| ATOM | 192 | CG2 | THR A | 425 | 37.045 | 30.974 | 57.854 | 1.00 | 51.68 | C |

| | | | | | | | | | | | |
|---|---|---|---|---|---|---|---|---|---|---|---|
| ANISOU | 192 | CG2 | THR A | 425 | 6550 | 6545 | 6540 | −14 | 56 | 50 | C |

| | | | | | | | | | | |
|---|---|---|---|---|---|---|---|---|---|---|
| ATOM | 193 | N | GLN A | 426 | 33.658 | 27.974 | 58.466 | 1.00 | 42.17 | N |

| | | | | | | | | | | | |
|---|---|---|---|---|---|---|---|---|---|---|---|
| ANISOU | 193 | N | GLN A | 426 | 5411 | 5391 | 5223 | −37 | 118 | 127 | N |

| | | | | | | | | | | |
|---|---|---|---|---|---|---|---|---|---|---|
| ATOM | 194 | CA | GLN A | 426 | 32.982 | 26.855 | 59.105 | 1.00 | 39.41 | C |

| | | | | | | | | | | | |
|---|---|---|---|---|---|---|---|---|---|---|---|
| ANISOU | 194 | CA | GLN A | 426 | 5005 | 5086 | 4884 | 37 | −3 | −56 | C |

| | | | | | | | | | | |
|---|---|---|---|---|---|---|---|---|---|---|
| ATOM | 195 | C | GLN A | 426 | 33.993 | 26.128 | 59.982 | 1.00 | 37.10 | C |

| | | | | | | | | | | | |
|---|---|---|---|---|---|---|---|---|---|---|---|
| ANISOU | 195 | C | GLN A | 426 | 4776 | 4805 | 4515 | −74 | 159 | −55 | C |

| | | | | | | | | | | |
|---|---|---|---|---|---|---|---|---|---|---|
| ATOM | 196 | O | GLN A | 426 | 35.066 | 25.714 | 59.503 | 1.00 | 37.82 | O |

| | | | | | | | | | | | |
|---|---|---|---|---|---|---|---|---|---|---|---|
| ANISOU | 196 | O | GLN A | 426 | 4897 | 4908 | 4565 | −1 | 262 | −142 | O |

| | | | | | | | | | | |
|---|---|---|---|---|---|---|---|---|---|---|
| ATOM | 197 | CB | GLN A | 426 | 32.450 | 25.920 | 58.005 | 1.00 | 39.00 | C |

| | | | | | | | | | | | |
|---|---|---|---|---|---|---|---|---|---|---|---|
| ANISOU | 197 | CB | GLN A | 426 | 4990 | 5007 | 4823 | −6 | 80 | −25 | C |

| | | | | | | | | | | |
|---|---|---|---|---|---|---|---|---|---|---|
| ATOM | 198 | CG | GLN A | 426 | 31.402 | 24.930 | 58.474 | 1.00 | 39.95 | C |

| | | | | | | | | | | | |
|---|---|---|---|---|---|---|---|---|---|---|---|
| ANISOU | 198 | CG | GLN A | 426 | 5175 | 4998 | 5006 | −84 | 66 | −24 | C |

| | | | | | | | | | | |
|---|---|---|---|---|---|---|---|---|---|---|
| ATOM | 199 | CD | GLN A | 426 | 31.088 | 23.834 | 57.460 | 1.00 | 39.53 | C |

| | | | | | | | | | | | |
|---|---|---|---|---|---|---|---|---|---|---|---|
| ANISOU | 199 | Cd | GLN A | 426 | 5106 | 5002 | 4912 | −5 | 105 | −14 | C |

| | | | | | | | | | | |
|---|---|---|---|---|---|---|---|---|---|---|
| ATOM | 200 | OE1 | GLN A | 426 | 31.921 | 22.931 | 57.334 | 1.00 | 39.47 | O |

| | | | | | | | | | | | |
|---|---|---|---|---|---|---|---|---|---|---|---|
| ANISOU | 200 | OE1 | GLN A | 426 | 5229 | 4871 | 4896 | −25 | 72 | −23 | O |

| | | | | | | | | | | |
|---|---|---|---|---|---|---|---|---|---|---|
| ATOM | 201 | NE2 | GLN A | 426 | 29.942 | 23.904 | 56.798 | 1.00 | 39.22 | N |

| | | | | | | | | | | | |
|---|---|---|---|---|---|---|---|---|---|---|---|
| ANISOU | 201 | NE2 | GLN A | 426 | 5148 | 4927 | 4828 | −79 | 111 | −10 | N |

| | | | | | | | | | | |
|---|---|---|---|---|---|---|---|---|---|---|
| ATOM | 202 | N | LEU A | 427 | 33.732 | 25.962 | 61.274 | 1.00 | 33.29 | N |

| | | | | | | | | | | | |
|---|---|---|---|---|---|---|---|---|---|---|---|
| ANISOU | 202 | N | LEU A | 427 | 4191 | 4150 | 4307 | 0 | 87 | −103 | N |

| | | | | | | | | | | |
|---|---|---|---|---|---|---|---|---|---|---|
| ATOM | 203 | CA | LEU A | 427 | 34.639 | 25.301 | 62.198 | 1.00 | 31.60 | C |

| | | | | | | | | | | | |
|---|---|---|---|---|---|---|---|---|---|---|---|
| ANISOU | 203 | CA | LEU A | 427 | 4120 | 3952 | 3935 | −70 | 202 | −138 | C |

| | | | | | | | | | | |
|---|---|---|---|---|---|---|---|---|---|---|
| ATOM | 204 | C | LEU A | 427 | 34.269 | 23.842 | 62.475 | 1.00 | 30.46 | C |

| | | | | | | | | | | | |
|---|---|---|---|---|---|---|---|---|---|---|---|
| ANISOU | 204 | C | LEU A | 427 | 3987 | 3948 | 3639 | 13 | 129 | −45 | C |

| | | | | | | | | | | |
|---|---|---|---|---|---|---|---|---|---|---|
| ATOM | 205 | O | LEU A | 427 | 35.135 | 23.106 | 62.938 | 1.00 | 30.00 | O |

| | | | | | | | | | | | |
|---|---|---|---|---|---|---|---|---|---|---|---|
| ANISOU | 205 | O | LEU A | 427 | 3925 | 3879 | 3595 | 168 | 301 | −167 | O |

| | | | | | | | | | | |
|---|---|---|---|---|---|---|---|---|---|---|
| ATOM | 206 | CB | LEU A | 427 | 34.701 | 26.001 | 63.560 | 1.00 | 30.35 | C |

| | | | | | | | | | | | |
|---|---|---|---|---|---|---|---|---|---|---|---|
| ANISOU | 206 | CB | LEU A | 427 | 3809 | 3923 | 3800 | −1 | 112 | −28 | C |

| | | | | | | | | | | |
|---|---|---|---|---|---|---|---|---|---|---|
| ATOM | 207 | CG | LEU A | 427 | 35.428 | 27.332 | 63.595 | 1.00 | 30.36 | C |

| | | | | | | | | | | | |
|---|---|---|---|---|---|---|---|---|---|---|---|
| ANISOU | 207 | CG | LEU A | 427 | 3871 | 3888 | 3779 | 20 | 61 | −68 | C |

| | | | | | | | | | | |
|---|---|---|---|---|---|---|---|---|---|---|
| ATOM | 208 | CD1 | LEU A | 427 | 35.288 | 27.954 | 64.974 | 1.00 | 29.52 | C |

| | | | | | | | | | | | |
|---|---|---|---|---|---|---|---|---|---|---|---|
| ANISOU | 208 | CD1 | LEU A | 427 | 3708 | 3678 | 3830 | 1 | 87 | −102 | C |

| | | | | | | | | | | |
|---|---|---|---|---|---|---|---|---|---|---|
| ATOM | 209 | CD2 | LEU A | 427 | 36.894 | 27.140 | 63.235 | 1.00 | 30.35 | C |

| | | | | | | | | | | | |
|---|---|---|---|---|---|---|---|---|---|---|---|
| ANISOU | 209 | CD2 | LEU A | 427 | 3901 | 3837 | 3792 | −12 | 124 | −47 | C |

| | | | | | | | | | | |
|---|---|---|---|---|---|---|---|---|---|---|
| ATOM | 210 | N | MET A | 428 | 32.988 | 23.532 | 62.301 | 1.00 | 29.77 | N |

| | | | | | | | | | | | |
|---|---|---|---|---|---|---|---|---|---|---|---|
| ANISOU | 210 | N | MET A | 428 | 3951 | 3747 | 3615 | 20 | 240 | −87 | N |

| | | | | | | | | | | |
|---|---|---|---|---|---|---|---|---|---|---|
| ATOM | 211 | CA | MET A | 428 | 32.508 | 22.182 | 62.580 | 1.00 | 29.75 | C |

| | | | | | | | | | | | |
|---|---|---|---|---|---|---|---|---|---|---|---|
| ANISOU | 211 | CA | MET A | 428 | 3885 | 3803 | 3617 | −2 | 150 | −45 | C |

| | | | | | | | | | | |
|---|---|---|---|---|---|---|---|---|---|---|
| ATOM | 212 | C | MET A | 428 | 31.203 | 21.991 | 61.818 | 1.00 | 30.03 | C |

| | | | | | | | | | | | |
|---|---|---|---|---|---|---|---|---|---|---|---|
| ANISOU | 212 | C | MET A | 428 | 3819 | 3836 | 3756 | −31 | 133 | −27 | C |

| | | | | | | | | | | |
|---|---|---|---|---|---|---|---|---|---|---|
| ATOM | 213 | O | MET A | 428 | 30.451 | 22.931 | 61.548 | 1.00 | 28.48 | O |

| | | | | | | | | | | | |
|---|---|---|---|---|---|---|---|---|---|---|---|
| ANISOU | 213 | O | MET A | 428 | 3602 | 3655 | 3564 | −152 | 286 | −228 | O |

| | | | | | | | | | | |
|---|---|---|---|---|---|---|---|---|---|---|
| ATOM | 214 | CB | MET A | 428 | 32.292 | 22.019 | 64.088 | 1.00 | 29.26 | C |

| | | | | | | | | | | | |
|---|---|---|---|---|---|---|---|---|---|---|---|
| ANISOU | 214 | CB | MET A | 428 | 3881 | 3633 | 3605 | 26 | 154 | −38 | C |

| | | | | | | | | | | |
|---|---|---|---|---|---|---|---|---|---|---|
| ATOM | 215 | CG | MET A | 428 | 32.239 | 20.562 | 64.570 | 1.00 | 28.39 | C |

| | | | | | | | | | | | |
|---|---|---|---|---|---|---|---|---|---|---|---|
| ANISOU | 215 | CG | MET A | 428 | 3766 | 3616 | 3407 | 31 | 240 | −80 | C |

| | | | | | | | | | | |
|---|---|---|---|---|---|---|---|---|---|---|
| ATOM | 216 | SD | MET A | 428 | 31.278 | 20.440 | 66.111 | 1.00 | 25.70 | S |

| | | | | | | | | | | | |
|---|---|---|---|---|---|---|---|---|---|---|---|
| ANISOU | 216 | SD | MET A | 428 | 3358 | 3041 | 3368 | 36 | 234 | −505 | S |

| | | | | | | | | | | |
|---|---|---|---|---|---|---|---|---|---|---|
| ATOM | 217 | CE | MET A | 428 | 29.628 | 20.532 | 65.423 | 1.00 | 26.43 | C |

| | | | | | | | | | | | |
|---|---|---|---|---|---|---|---|---|---|---|---|
| ANISOU | 217 | CE | MET A | 428 | 3500 | 3193 | 3348 | 39 | 38 | −197 | C |

| | | | | | | | | | | |
|---|---|---|---|---|---|---|---|---|---|---|
| ATOM | 218 | N | ASN A | 429 | 30.969 | 20.760 | 61.384 | 1.00 | 30.83 | N |

| | | | | | | | | | | | |
|---|---|---|---|---|---|---|---|---|---|---|---|
| ANISOU | 218 | N | ASN A | 429 | 3955 | 3869 | 3892 | 41 | 153 | −130 | N |

| | | | | | | | | | | |
|---|---|---|---|---|---|---|---|---|---|---|
| ATOM | 219 | CA | ASN A | 429 | 29.748 | 20.434 | 60.663 | 1.00 | 31.92 | C |

| | | | | | | | | | | | |
|---|---|---|---|---|---|---|---|---|---|---|---|
| ANISOU | 219 | CA | ASN A | 429 | 3919 | 4050 | 4161 | 85 | 98 | −89 | C |

| | | | | | | | | | | |
|---|---|---|---|---|---|---|---|---|---|---|
| ATOM | 220 | C | ASN A | 429 | 29.519 | 18.945 | 60.884 | 1.00 | 31.94 | C |

| | | | | | | | | | | | |
|---|---|---|---|---|---|---|---|---|---|---|---|
| ANISOU | 220 | C | ASN A | 429 | 3957 | 4076 | 4104 | 32 | 119 | −53 | C |

| | | | | | | | | | | |
|---|---|---|---|---|---|---|---|---|---|---|
| ATOM | 221 | O | ASN A | 429 | 30.201 | 18.120 | 60.258 | 1.00 | 33.09 | O |

| | | | | | | | | | | | |
|---|---|---|---|---|---|---|---|---|---|---|---|
| ANISOU | 221 | O | ASN A | 429 | 4188 | 4226 | 4160 | 42 | 182 | −215 | O |

| | | | | | | | | | | |
|---|---|---|---|---|---|---|---|---|---|---|
| ATOM | 222 | CB | ASN A | 429 | 29.797 | 20.803 | 59.196 | 1.00 | 34.07 | C |

| | | | | | | | | | | | |
|---|---|---|---|---|---|---|---|---|---|---|---|
| ANISOU | 222 | CB | ASN A | 429 | 4403 | 4315 | 4229 | 74 | 142 | −37 | C |

| | | | | | | | | | | |
|---|---|---|---|---|---|---|---|---|---|---|
| ATOM | 223 | CG | ASN A | 429 | 28.451 | 20.675 | 58.523 | 1.00 | 36.74 | C |

| | | | | | | | | | | | |
|---|---|---|---|---|---|---|---|---|---|---|---|
| ANISOU | 223 | CG | ASN A | 429 | 4631 | 4747 | 4583 | 26 | −52 | −56 | C |

| | | | | | | | | | | |
|---|---|---|---|---|---|---|---|---|---|---|
| ATOM | 224 | OD1 | ASN A | 429 | 27.419 | 20.747 | 59.208 | 1.00 | 37.57 | O |

| | | | | | | | | | | | |
|---|---|---|---|---|---|---|---|---|---|---|---|
| ANISOU | 224 | OD1 | ASN A | 429 | 4722 | 4952 | 4602 | 43 | −13 | −104 | O |

| | | | | | | | | | | |
|---|---|---|---|---|---|---|---|---|---|---|
| ATOM | 225 | ND2 | ASN A | 429 | 28.429 | 20.485 | 57.210 | 1.00 | 37.30 | N |

| | | | | | | | | | | | |
|---|---|---|---|---|---|---|---|---|---|---|---|
| ANISOU | 225 | ND2 | ASN A | 429 | 4892 | 4698 | 4583 | 83 | 44 | −16 | N |

| | | | | | | | | | | |
|---|---|---|---|---|---|---|---|---|---|---|
| ATOM | 226 | N | ARG A | 430 | 28.680 | 18.646 | 61.861 | 1.00 | 30.73 | N |

| | | | | | | | | | | | |
|---|---|---|---|---|---|---|---|---|---|---|---|
| ANISOU | 226 | N | ARG A | 430 | 3821 | 3885 | 3969 | 71 | 102 | −123 | N |

| | | | | | | | | | | |
|---|---|---|---|---|---|---|---|---|---|---|
| ATOM | 227 | CA | ARG A | 430 | 28.476 | 17.228 | 62.191 | 1.00 | 32.06 | C |

| | | | | | | | | | | | |
|---|---|---|---|---|---|---|---|---|---|---|---|
| ANISOU | 227 | CA | ARG A | 430 | 4078 | 3965 | 4140 | −24 | 191 | −105 | C |

| | | | | | | | | | | |
|---|---|---|---|---|---|---|---|---|---|---|
| ATOM | 228 | C | ARG A | 430 | 27.136 | 17.026 | 62.862 | 1.00 | 32.39 | C |

| | | | | | | | | | | | |
|---|---|---|---|---|---|---|---|---|---|---|---|
| ANISOU | 228 | C | ARG A | 430 | 3955 | 4076 | 4278 | 5 | 76 | −137 | C |

| | | | | | | | | | | |
|---|---|---|---|---|---|---|---|---|---|---|
| ATOM | 229 | O | ARG A | 430 | 26.751 | 17.893 | 63.646 | 1.00 | 31.05 | O |

| | | | | | | | | | | | |
|---|---|---|---|---|---|---|---|---|---|---|---|
| ANISOU | 229 | O | ARG A | 430 | 3572 | 3968 | 4258 | 4 | 35 | −149 | O |

| | | | | | | | | | | |
|---|---|---|---|---|---|---|---|---|---|---|
| ATOM | 230 | CB | ARG A | 430 | 29.612 | 16.842 | 63.128 | 1.00 | 33.68 | C |

| | | | | | | | | | | | |
|---|---|---|---|---|---|---|---|---|---|---|---|
| ANISOU | 230 | CB | ARG A | 430 | 4203 | 4287 | 4308 | −62 | 46 | −42 | C |

| | | | | | | | | | | |
|---|---|---|---|---|---|---|---|---|---|---|
| ATOM | 231 | CG | ARG A | 430 | 29.598 | 15.411 | 63.653 | 1.00 | 36.38 | C |

| | | | | | | | | | | | |
|---|---|---|---|---|---|---|---|---|---|---|---|
| ANISOU | 231 | CG | ARG A | 430 | 4689 | 4417 | 4717 | −66 | 67 | 60 | C |

| | | | | | | | | | | |
|---|---|---|---|---|---|---|---|---|---|---|
| ATOM | 232 | CD | ARG A | 430 | 30.321 | 14.483 | 62.727 | 1.00 | 38.04 | C |

| | | | | | | | | | | | |
|---|---|---|---|---|---|---|---|---|---|---|---|
| ANISOU | 232 | CD | ARG A | 430 | 4850 | 4705 | 4899 | −11 | 74 | −69 | C |

| | | | | | | | | | | |
|---|---|---|---|---|---|---|---|---|---|---|
| ATOM | 233 | NE | ARG A | 430 | 31.735 | 14.848 | 62.498 | 1.00 | 39.38 | N |

| | | | | | | | | | | | |
|---|---|---|---|---|---|---|---|---|---|---|---|
| ANISOU | 233 | NE | ARG A | 430 | 4877 | 4985 | 5100 | −56 | 98 | −43 | N |

| | | | | | | | | | | |
|---|---|---|---|---|---|---|---|---|---|---|
| ATOM | 234 | CZ | ARG A | 430 | 32.449 | 13.997 | 61.741 | 1.00 | 41.34 | C |

| | | | | | | | | | | | |
|---|---|---|---|---|---|---|---|---|---|---|---|
| ANISOU | 234 | CZ | ARG A | 430 | 5178 | 5209 | 5320 | 50 | 112 | −127 | C |

| | | | | | | | | | | |
|---|---|---|---|---|---|---|---|---|---|---|
| ATOM | 235 | NH1 | ARG A | 430 | 31.839 | 12.904 | 61.272 | 1.00 | 41.92 | N |

| | | | | | | | | | | | |
|---|---|---|---|---|---|---|---|---|---|---|---|
| ANISOU | 235 | NH1 | ARG A | 430 | 5264 | 5295 | 5370 | −5 | 26 | −123 | N |

| | | | | | | | | | | |
|---|---|---|---|---|---|---|---|---|---|---|
| ATOM | 236 | NH2 | ARG A | 430 | 33.718 | 14.264 | 61.478 | 1.00 | 41.05 | N |

| | | | | | | | | | | | |
|---|---|---|---|---|---|---|---|---|---|---|---|
| ANISOU | 236 | NH2 | ARG A | 430 | 5153 | 5243 | 5199 | 57 | 78 | −178 | N |

| | | | | | | | | | | |
|---|---|---|---|---|---|---|---|---|---|---|
| ATOM | 237 | N | ASP A | 431 | 26.427 | 15.931 | 62.583 | 1.00 | 32.85 | N |

| | | | | | | | | | | | |
|---|---|---|---|---|---|---|---|---|---|---|---|
| ANISOU | 237 | N | ASP A | 431 | 4151 | 4003 | 4327 | 43 | 10 | −140 | N |

| | | | | | | | | | | |
|---|---|---|---|---|---|---|---|---|---|---|
| ATOM | 238 | CA | ASP A | 431 | 25.164 | 15.664 | 63.275 | 1.00 | 33.72 | C |

| | | | | | | | | | | | |
|---|---|---|---|---|---|---|---|---|---|---|---|
| ANISOU | 238 | CA | ASP A | 431 | 4150 | 4041 | 4621 | 80 | 10 | −74 | C |

| | | | | | | | | | | |
|---|---|---|---|---|---|---|---|---|---|---|
| ATOM | 239 | C | ASP A | 431 | 24.129 | 16.766 | 63.080 | 1.00 | 31.60 | C |

| | | | | | | | | | | | |
|---|---|---|---|---|---|---|---|---|---|---|---|
| ANISOU | 239 | C | ASP A | 431 | 3857 | 4027 | 4123 | −22 | −44 | −66 | C |

| | | | | | | | | | | |
|---|---|---|---|---|---|---|---|---|---|---|
| ATOM | 240 | O | ASP A | 431 | 23.332 | 16.998 | 63.995 | 1.00 | 30.44 | O |

| | | | | | | | | | | | |
|---|---|---|---|---|---|---|---|---|---|---|---|
| ANISOU | 240 | O | ASP A | 431 | 3767 | 3721 | 4077 | 55 | −132 | −77 | O |

| | | | | | | | | | | |
|---|---|---|---|---|---|---|---|---|---|---|
| ATOM | 241 | CB | ASP A | 431 | 25.525 | 15.576 | 64.761 | 1.00 | 37.31 | C |

| | | | | | | | | | | | |
|---|---|---|---|---|---|---|---|---|---|---|---|
| ANISOU | 241 | CB | ASP A | 431 | 4767 | 4659 | 4751 | 114 | −111 | −60 | C |

| | | | | | | | | | | |
|---|---|---|---|---|---|---|---|---|---|---|
| ATOM | 242 | CG | ASP A | 431 | 24.609 | 14.787 | 65.650 | 1.00 | 41.39 | C |

| | | | | | | | | | | | |
|---|---|---|---|---|---|---|---|---|---|---|---|
| ANISOU | 242 | CG | ASP A | 431 | 5255 | 5147 | 5323 | −76 | 98 | 70 | C |

| | | | | | | | | | | |
|---|---|---|---|---|---|---|---|---|---|---|
| ATOM | 243 | OD1 | ASP A | 431 | 23.647 | 14.173 | 65.134 | 1.00 | 42.79 | O |

| | | | | | | | | | | | |
|---|---|---|---|---|---|---|---|---|---|---|---|
| ANISOU | 243 | OD1 | ASP A | 431 | 5319 | 5455 | 5483 | −86 | −56 | 5 | O |

| | | | | | | | | | | |
|---|---|---|---|---|---|---|---|---|---|---|
| ATOM | 244 | OD2 | ASP A | 431 | 24.864 | 14.779 | 66.885 | 1.00 | 43.70 | O |

| | | | | | | | | | | | |
|---|---|---|---|---|---|---|---|---|---|---|---|
| ANISOU | 244 | OD2 | ASP A | 431 | 5720 | 5403 | 5480 | −44 | −113 | 12 | O |

| | | | | | | | | | | |
|---|---|---|---|---|---|---|---|---|---|---|
| ATOM | 245 | N | ASN A | 432 | 24.144 | 17.410 | 61.939 | 1.00 | 29.57 | N |

| | | | | | | | | | | | |
|---|---|---|---|---|---|---|---|---|---|---|---|
| ANISOU | 245 | N | ASN A | 432 | 3550 | 3664 | 4020 | 14 | −83 | −160 | N |

| | | | | | | | | | | |
|---|---|---|---|---|---|---|---|---|---|---|
| ATOM | 246 | CA | ASN A | 432 | 23.245 | 18.491 | 61.558 | 1.00 | 30.19 | C |

| | | | | | | | | | | | |
|---|---|---|---|---|---|---|---|---|---|---|---|
| ANISOU | 246 | CA | ASN A | 432 | 3732 | 3756 | 3981 | 62 | −93 | −159 | C |

| | | | | | | | | | | |
|---|---|---|---|---|---|---|---|---|---|---|
| ATOM | 247 | C | ASN A | 432 | 23.346 | 19.756 | 62.389 | 1.00 | 28.16 | C |

| | | | | | | | | | | | |
|---|---|---|---|---|---|---|---|---|---|---|---|
| ANISOU | 247 | C | ASN A | 432 | 3365 | 3547 | 3789 | 81 | −36 | −40 | C |

| | | | | | | | | | | |
|---|---|---|---|---|---|---|---|---|---|---|
| ATOM | 248 | O | ASN A | 432 | 22.390 | 20.483 | 62.639 | 1.00 | 25.96 | O |

| | | | | | | | | | | | |
|---|---|---|---|---|---|---|---|---|---|---|---|
| ANISOU | 248 | O | ASN A | 432 | 2946 | 3382 | 3537 | −156 | −248 | −88 | O |

| | | | | | | | | | | |
|---|---|---|---|---|---|---|---|---|---|---|
| ATOM | 249 | CB | ASN A | 432 | 21.786 | 18.014 | 61.397 | 1.00 | 31.82 | C |

| | | | | | | | | | | | |
|---|---|---|---|---|---|---|---|---|---|---|---|
| ANISOU | 249 | CB | ASN A | 432 | 3892 | 4041 | 4158 | −62 | −136 | −102 | C |

| | | | | | | | | | | |
|---|---|---|---|---|---|---|---|---|---|---|
| ATOM | 250 | CG | ASN A | 432 | 21.481 | 18.031 | 59.900 | 1.00 | 33.21 | C |

| | | | | | | | | | | | |
|---|---|---|---|---|---|---|---|---|---|---|---|
| ANISOU | 250 | CG | ASN A | 432 | 4242 | 4239 | 4136 | 44 | −48 | −67 | C |

| | | | | | | | | | | |
|---|---|---|---|---|---|---|---|---|---|---|
| ATOM | 251 | OD1 | ASN A | 432 | 21.410 | 16.952 | 59.304 | 1.00 | 34.07 | O |

| | | | | | | | | | | | |
|---|---|---|---|---|---|---|---|---|---|---|---|
| ANISOU | 251 | OD1 | ASN A | 432 | 4423 | 4318 | 4204 | 96 | −199 | −152 | O |

| | | | | | | | | | | |
|---|---|---|---|---|---|---|---|---|---|---|
| ATOM | 252 | ND2 | ASN A | 432 | 21.340 | 19.228 | 59.338 | 1.00 | 33.00 | N |

| | | | | | | | | | | | |
|---|---|---|---|---|---|---|---|---|---|---|---|
| ANISON | 252 | ND2 | ASN A | 432 | 4252 | 4154 | 4133 | 101 | −12 | −187 | N |

| | | | | | | | | | | |
|---|---|---|---|---|---|---|---|---|---|---|
| ATOM | 253 | N | ILE A | 433 | 24.579 | 20.075 | 62.807 | 1.00 | 27.29 | N |

| | | | | | | | | | | | |
|---|---|---|---|---|---|---|---|---|---|---|---|
| ANISOU | 253 | N | ILE A | 433 | 3423 | 3473 | 3472 | 62 | 3 | −139 | N |

| | | | | | | | | | | |
|---|---|---|---|---|---|---|---|---|---|---|
| ATOM | 254 | CA | ILE A | 433 | 24.836 | 21.341 | 63.502 | 1.00 | 27.53 | C |

| | | | | | | | | | | | |
|---|---|---|---|---|---|---|---|---|---|---|---|
| ANISOU | 254 | CA | ILE A | 433 | 3519 | 3359 | 3584 | −38 | −108 | −2 | C |

| | | | | | | | | | | |
|---|---|---|---|---|---|---|---|---|---|---|
| ATOM | 255 | C | ILE A | 433 | 26.138 | 21.837 | 62.872 | 1.00 | 27.63 | C |

| | | | | | | | | | | | |
|---|---|---|---|---|---|---|---|---|---|---|---|
| ANISOU | 255 | C | ILE A | 433 | 3655 | 3447 | 3396 | −19 | −33 | −148 | C |

| | | | | | | | | | | |
|---|---|---|---|---|---|---|---|---|---|---|
| ATOM | 256 | O | ILE A | 433 | 27.191 | 21.213 | 62.932 | 1.00 | 28.12 | O |

| | | | | | | | | | | | |
|---|---|---|---|---|---|---|---|---|---|---|---|
| ANISOU | 256 | O | ILE A | 433 | 3583 | 3550 | 3550 | −70 | 40 | −79 | O |

| | | | | | | | | | | |
|---|---|---|---|---|---|---|---|---|---|---|
| ATOM | 257 | CB | ILE A | 433 | 24.851 | 21.252 | 65.009 | 1.00 | 28.90 | C |

| | | | | | | | | | | | |
|---|---|---|---|---|---|---|---|---|---|---|---|
| ANISOU | 257 | CB | ILE A | 433 | 3705 | 3647 | 3628 | 7 | 6 | −121 | C |

| | | | | | | | | | | |
|---|---|---|---|---|---|---|---|---|---|---|
| ATOM | 258 | CG1 | ILE A | 433 | 25.119 | 22.600 | 65.684 | 1.00 | 29.59 | C |

| | | | | | | | | | | | |
|---|---|---|---|---|---|---|---|---|---|---|---|
| ANISOU | 258 | CG1 | ILE A | 433 | 3759 | 3694 | 3791 | −63 | −86 | −98 | C |

| | | | | | | | | | | |
|---|---|---|---|---|---|---|---|---|---|---|
| ATOM | 259 | CG2 | ILE A | 433 | 25.868 | 20.240 | 65.532 | 1.00 | 30.05 | C |

| | | | | | | | | | | | |
|---|---|---|---|---|---|---|---|---|---|---|---|
| ANISOU | 259 | CG2 | ILE A | 433 | 3842 | 3855 | 3721 | 57 | −44 | −70 | C |

| | | | | | | | | | | |
|---|---|---|---|---|---|---|---|---|---|---|
| ATOM | 260 | CD1 | ILE A | 433 | 24.084 | 23.673 | 65.541 | 1.00 | 29.82 | C |

| | | | | | | | | | | | |
|---|---|---|---|---|---|---|---|---|---|---|---|
| ANISOU | 260 | CD1 | ILE A | 433 | 3808 | 3683 | 3840 | −39 | 12 | −113 | C |

| | | | | | | | | | | |
|---|---|---|---|---|---|---|---|---|---|---|
| ATOM | 261 | N | GLU A | 434 | 25.969 | 22.950 | 62.193 | 1.00 | 26.98 | N |

| | | | | | | | | | | | |
|---|---|---|---|---|---|---|---|---|---|---|---|
| ANISOU | 261 | N | GLU A | 434 | 3817 | 3461 | 2975 | −28 | −36 | −222 | N |

| | | | | | | | | | | |
|---|---|---|---|---|---|---|---|---|---|---|
| ATOM | 262 | CA | GLU A | 434 | 27.087 | 23.636 | 61.549 | 1.00 | 29.04 | C |

| | | | | | | | | | | | |
|---|---|---|---|---|---|---|---|---|---|---|---|
| ANISOU | 262 | CA | GLU A | 434 | 3782 | 3621 | 3630 | −73 | −4 | −186 | C |

| | | | | | | | | | | |
|---|---|---|---|---|---|---|---|---|---|---|
| ATOM | 263 | C | GLU A | 434 | 27.496 | 24.805 | 62.448 | 1.00 | 26.87 | C |

| | | | | | | | | | | | |
|---|---|---|---|---|---|---|---|---|---|---|---|
| ANISOU | 263 | C | GLU A | 434 | 3410 | 3498 | 3302 | 66 | −10 | −121 | C |

| | | | | | | | | | | |
|---|---|---|---|---|---|---|---|---|---|---|
| ATOM | 264 | O | GLU A | 434 | 26.605 | 25.529 | 62.918 | 1.00 | 25.79 | O |

| | | | | | | | | | | | |
|---|---|---|---|---|---|---|---|---|---|---|---|
| ANISOU | 264 | O | GLU A | 434 | 3384 | 3312 | 3102 | 1 | −171 | −307 | O |

| | | | | | | | | | | |
|---|---|---|---|---|---|---|---|---|---|---|
| ATOM | 265 | CB | GLU A | 434 | 26.555 | 24.135 | 60.208 | 1.00 | 32.25 | C |

| | | | | | | | | | | | |
|---|---|---|---|---|---|---|---|---|---|---|---|
| ANISOU | 265 | CB | GLU A | 434 | 4288 | 4141 | 3824 | 16 | −151 | −100 | C |

| | | | | | | | | | | |
|---|---|---|---|---|---|---|---|---|---|---|
| ATOM | 266 | CG | GLU A | 434 | 27.504 | 25.003 | 59.425 | 1.00 | 35.93 | C |

| | | | | | | | | | | | |
|---|---|---|---|---|---|---|---|---|---|---|---|
| ANISOU | 266 | CG | GLU A | 434 | 4594 | 4572 | 4486 | −111 | 112 | 6 | C |

| | | | | | | | | | | |
|---|---|---|---|---|---|---|---|---|---|---|
| ATOM | 267 | CD | GLU A | 434 | 26.879 | 25.554 | 58.153 | 1.00 | 38.19 | C |

| | | | | | | | | | | | |
|---|---|---|---|---|---|---|---|---|---|---|---|
| ANISOU | 267 | CD | GLU A | 434 | 4868 | 4933 | 4709 | 16 | −31 | 89 | C |

| | | | | | | | | | | |
|---|---|---|---|---|---|---|---|---|---|---|
| ATOM | 268 | OE1 | GLU A | 434 | 25.651 | 25.820 | 58.096 | 1.00 | 39.69 | O |

| | | | | | | | | | | | |
|---|---|---|---|---|---|---|---|---|---|---|---|
| ANISOU | 268 | OE1 | GLU A | 434 | 4945 | 5113 | 5020 | 63 | 136 | 109 | O |

| | | | | | | | | | | |
|---|---|---|---|---|---|---|---|---|---|---|
| ATOM | 269 | OE2 | GLU A | 434 | 27.647 | 25.674 | 57.183 | 1.00 | 39.34 | O |

| | | | | | | | | | | | |
|---|---|---|---|---|---|---|---|---|---|---|---|
| ANISOU | 269 | OE2 | GLU A | 434 | 4893 | 5161 | 4894 | 4 | 80 | 176 | O |

| | | | | | | | | | | |
|---|---|---|---|---|---|---|---|---|---|---|
| ATOM | 270 | N | ILE A | 435 | 28.784 | 24.958 | 62.728 | 1.00 | 24.78 | N |

| | | | | | | | | | | | |
|---|---|---|---|---|---|---|---|---|---|---|---|
| ANISOU | 270 | N | ILE A | 435 | 3317 | 3072 | 3025 | −85 | 114 | −224 | N |

| | | | | | | | | | | |
|---|---|---|---|---|---|---|---|---|---|---|
| ATOM | 271 | CA | ILE A | 435 | 29.257 | 26.019 | 63.625 | 1.00 | 24.58 | C |

| | | | | | | | | | | | |
|---|---|---|---|---|---|---|---|---|---|---|---|
| ANISOU | 271 | CA | ILE A | 435 | 3236 | 3053 | 3049 | −50 | 69 | −179 | C |

| | | | | | | | | | | |
|---|---|---|---|---|---|---|---|---|---|---|
| ATOM | 272 | C | ILE A | 435 | 30.294 | 26.872 | 62.886 | 1.00 | 24.98 | C |

| | | | | | | | | | | | |
|---|---|---|---|---|---|---|---|---|---|---|---|
| ANISOU | 272 | C | ILE A | 435 | 3223 | 3170 | 3098 | −118 | −12 | −130 | C |

| | | | | | | | | | | |
|---|---|---|---|---|---|---|---|---|---|---|
| ATOM | 273 | O | ILE A | 435 | 31.274 | 26.306 | 62.400 | 1.00 | 26.66 | O |

| | | | | | | | | | | | |
|---|---|---|---|---|---|---|---|---|---|---|---|
| ANISOU | 273 | O | ILE A | 435 | 3488 | 3317 | 3325 | −43 | 95 | −112 | O |

| | | | | | | | | | | |
|---|---|---|---|---|---|---|---|---|---|---|
| ATOM | 274 | CB | ILE A | 435 | 29.821 | 25.456 | 64.940 | 1.00 | 24.87 | C |

| | | | | | | | | | | | |
|---|---|---|---|---|---|---|---|---|---|---|---|
| ANISOU | 274 | CB | ILE A | 435 | 3248 | 3087 | 3116 | 1 | 59 | −187 | C |

| | | | | | | | | | | |
|---|---|---|---|---|---|---|---|---|---|---|
| ATOM | 275 | CG1 | ILE A | 435 | 28.785 | 24.576 | 65.663 | 1.00 | 25.95 | C |

| | | | | | | | | | | | |
|---|---|---|---|---|---|---|---|---|---|---|---|
| ANISOU | 275 | CG1 | ILE A | 435 | 3476 | 3188 | 3195 | −106 | −43 | −67 | C |

| | | | | | | | | | | |
|---|---|---|---|---|---|---|---|---|---|---|
| ATOM | 276 | CG2 | ILE A | 435 | 30.276 | 26.587 | 65.861 | 1.00 | 24.85 | C |

| | | | | | | | | | | | |
|---|---|---|---|---|---|---|---|---|---|---|---|
| ANISOU | 276 | CG2 | ILE A | 435 | 3244 | 3188 | 3010 | 1 | 104 | −180 | C |

| | | | | | | | | | | |
|---|---|---|---|---|---|---|---|---|---|---|
| ATOM | 277 | CD1 | ILE A | 435 | 29.256 | 23.840 | 66.869 | 1.00 | 26.22 | C |

| | | | | | | | | | | | |
|---|---|---|---|---|---|---|---|---|---|---|---|
| ANISOU | 277 | CD1 | ILE A | 435 | 3513 | 3250 | 3198 | −70 | 21 | −6 | C |

| | | | | | | | | | | |
|---|---|---|---|---|---|---|---|---|---|---|
| ATOM | 278 | N | THR A | 436 | 29.988 | 28.169 | 62.768 | 1.00 | 24.37 | N |

| | | | | | | | | | | | |
|---|---|---|---|---|---|---|---|---|---|---|---|
| ANISOU | 278 | N | THR A | 436 | 3273 | 3094 | 2894 | −150 | 114 | −28 | N |

| | | | | | | | | | | |
|---|---|---|---|---|---|---|---|---|---|---|
| ATOM | 279 | CA | THR A | 436 | 30.940 | 29.080 | 62.136 | 1.00 | 25.67 | C |

| | | | | | | | | | | | |
|---|---|---|---|---|---|---|---|---|---|---|---|
| ANISOU | 279 | CA | THR A | 436 | 3541 | 3121 | 3091 | −174 | 92 | 2 | C |

| | | | | | | | | | | |
|---|---|---|---|---|---|---|---|---|---|---|
| ATOM | 280 | C | THR A | 436 | 31.724 | 29.829 | 63.189 | 1.00 | 25.18 | C |

| | | | | | | | | | | | |
|---|---|---|---|---|---|---|---|---|---|---|---|
| ANISOU | 280 | C | THR A | 436 | 3136 | 3245 | 3187 | −221 | 131 | 93 | C |

| | | | | | | | | | | |
|---|---|---|---|---|---|---|---|---|---|---|
| ATOM | 281 | O | THR A | 436 | 31.430 | 29.739 | 64.379 | 1.00 | 24.31 | O |

| | | | | | | | | | | | |
|---|---|---|---|---|---|---|---|---|---|---|---|
| ANISOU | 281 | O | THR A | 436 | 3137 | 2996 | 3103 | −181 | 80 | 105 | O |

| | | | | | | | | | | |
|---|---|---|---|---|---|---|---|---|---|---|
| ATOM | 282 | CB | THR A | 436 | 30.201 | 30.153 | 61.300 | 1.00 | 27.65 | C |

| | | | | | | | | | | | |
|---|---|---|---|---|---|---|---|---|---|---|---|
| ANISOU | 282 | CB | THR A | 436 | 3643 | 3418 | 3444 | −45 | −68 | 111 | C |

| | | | | | | | | | | |
|---|---|---|---|---|---|---|---|---|---|---|
| ATOM | 283 | OG1 | THR A | 436 | 29.412 | 31.009 | 62.123 | 1.00 | 27.54 | O |

| | | | | | | | | | | | |
|---|---|---|---|---|---|---|---|---|---|---|---|
| ANISOU | 283 | OG1 | THR A | 436 | 3778 | 3251 | 3436 | −120 | 6 | 203 | O |

| | | | | | | | | | | |
|---|---|---|---|---|---|---|---|---|---|---|
| ATOM | 284 | CG2 | THR A | 436 | 29.291 | 29.450 | 60.283 | 1.00 | 28.64 | C |

| | | | | | | | | | | | |
|---|---|---|---|---|---|---|---|---|---|---|---|
| ANISOU | 284 | CG2 | THR A | 436 | 3783 | 3630 | 3468 | −86 | −130 | 107 | C |

| | | | | | | | | | | |
|---|---|---|---|---|---|---|---|---|---|---|
| ATOM | 285 | N | VAL A | 437 | 32.736 | 30.592 | 62.713 | 1.00 | 25.05 | N |

| | | | | | | | | | | | |
|---|---|---|---|---|---|---|---|---|---|---|---|
| ANISOU | 285 | N | VAL A | 437 | 3417 | 2954 | 3148 | −222 | 220 | 85 | N |

| | | | | | | | | | | |
|---|---|---|---|---|---|---|---|---|---|---|
| ATOM | 286 | CA | VAL A | 437 | 33.495 | 31.412 | 63.651 | 1.00 | 24.74 | C |

| | | | | | | | | | | | |
|---|---|---|---|---|---|---|---|---|---|---|---|
| ANISOU | 286 | CA | VAL A | 437 | 3024 | 3161 | 3215 | −268 | 253 | 135 | C |

| | | | | | | | | | | |
|---|---|---|---|---|---|---|---|---|---|---|
| ATOM | 287 | C | VAL A | 437 | 32.579 | 32.414 | 64.333 | 1.00 | 24.55 | C |

| | | | | | | | | | | | |
|---|---|---|---|---|---|---|---|---|---|---|---|
| ANISOU | 287 | C | VAL A | 437 | 3136 | 3094 | 3096 | −223 | 155 | 111 | C |

| | | | | | | | | | | |
|---|---|---|---|---|---|---|---|---|---|---|
| ATOM | 288 | O | VAL A | 437 | 32.779 | 32.653 | 65.524 | 1.00 | 23.46 | O |

| | | | | | | | | | | | |
|---|---|---|---|---|---|---|---|---|---|---|---|
| ANISOU | 288 | O | VAL A | 437 | 3069 | 2771 | 3072 | −143 | 210 | 83 | O |

| | | | | | | | | | | |
|---|---|---|---|---|---|---|---|---|---|---|
| ATOM | 289 | CB | VAL A | 437 | 34.603 | 32.190 | 62.859 | 1.00 | 26.10 | C |

| | | | | | | | | | | | |
|---|---|---|---|---|---|---|---|---|---|---|---|
| ANISOU | 289 | CB | VAL A | 437 | 3277 | 3382 | 3258 | −330 | 349 | 167 | C |

| | | | | | | | | | | |
|---|---|---|---|---|---|---|---|---|---|---|
| ATOM | 290 | CG1 | VAL A | 437 | 35.277 | 33.207 | 63.743 | 1.00 | 26.57 | C |

| | | | | | | | | | | | |
|---|---|---|---|---|---|---|---|---|---|---|---|
| ANISOU | 290 | CG1 | VAL A | 437 | 3524 | 3348 | 3223 | −185 | 313 | 65 | C |

| | | | | | | | | | | |
|---|---|---|---|---|---|---|---|---|---|---|
| ATOM | 291 | CG2 | VAL A | 437 | 35.602 | 31.101 | 62.489 | 1.00 | 28.12 | C |

| | | | | | | | | | | | |
|---|---|---|---|---|---|---|---|---|---|---|---|
| ANISOU | 291 | CG2 | VAL A | 437 | 3345 | 3737 | 3603 | −156 | 334 | 86 | C |

| | | | | | | | | | | |
|---|---|---|---|---|---|---|---|---|---|---|
| ATOM | 292 | N | ARG A | 438 | 31.613 | 32.996 | 63.616 | 1.00 | 24.20 | N |

| | | | | | | | | | | | |
|---|---|---|---|---|---|---|---|---|---|---|---|
| ANISOU | 292 | N | ARG A | 438 | 3218 | 2981 | 2995 | −163 | 220 | 160 | N |

| | | | | | | | | | | |
|---|---|---|---|---|---|---|---|---|---|---|
| ATOM | 293 | CA | ARG A | 438 | 30.692 | 33.944 | 64.248 | 1.00 | 24.68 | C |

| | | | | | | | | | | | |
|---|---|---|---|---|---|---|---|---|---|---|---|
| ANISOU | 293 | CA | ARG A | 438 | 3290 | 3086 | 3002 | −131 | 223 | 156 | C |

| | | | | | | | | | | |
|---|---|---|---|---|---|---|---|---|---|---|
| ATOM | 294 | C | ARG A | 438 | 29.978 | 33.277 | 65.434 | 1.00 | 23.38 | C |

| | | | | | | | | | | | |
|---|---|---|---|---|---|---|---|---|---|---|---|
| ANISOU | 294 | C | ARG A | 438 | 3311 | 2812 | 2762 | −89 | 125 | 87 | C |

| | | | | | | | | | | |
|---|---|---|---|---|---|---|---|---|---|---|
| ATOM | 295 | O | ARG A | 438 | 29.904 | 33.861 | 66.514 | 1.00 | 22.44 | O |

| | | | | | | | | | | | |
|---|---|---|---|---|---|---|---|---|---|---|---|
| ANISOU | 295 | O | ARG A | 438 | 3346 | 2506 | 2675 | −183 | 378 | 190 | O |

| | | | | | | | | | | |
|---|---|---|---|---|---|---|---|---|---|---|
| ATOM | 296 | CB | ARG A | 438 | 29.669 | 34.411 | 63.207 | 1.00 | 28.16 | C |

| | | | | | | | | | | | |
|---|---|---|---|---|---|---|---|---|---|---|---|
| ANISOU | 296 | CB | ARG A | 438 | 3611 | 3594 | 3495 | −64 | −42 | 294 | C |

| | | | | | | | | | | |
|---|---|---|---|---|---|---|---|---|---|---|
| ATOM | 297 | CG | ARG A | 438 | 28.713 | 35.451 | 63.723 | 1.00 | 31.14 | C |

| | | | | | | | | | | | |
|---|---|---|---|---|---|---|---|---|---|---|---|
| ANISOU | 297 | CG | ARG A | 438 | 4077 | 3779 | 3976 | −1 | 157 | 63 | C |

| | | | | | | | | | | |
|---|---|---|---|---|---|---|---|---|---|---|
| ATOM | 298 | CD | ARG A | 438 | 27.764 | 35.933 | 62.618 | 1.00 | 34.29 | C |

| | | | | | | | | | | | |
|---|---|---|---|---|---|---|---|---|---|---|---|
| ANISOU | 298 | CD | ARG A | 438 | 4372 | 4392 | 4265 | −53 | −72 | 187 | C |

| | | | | | | | | | | |
|---|---|---|---|---|---|---|---|---|---|---|
| ATOM | 299 | NE | ARG A | 438 | 26.851 | 36.917 | 63.189 | 1.00 | 37.27 | N |

| | | | | | | | | | | | |
|---|---|---|---|---|---|---|---|---|---|---|---|
| ANISOU | 299 | NE | ARG A | 438 | 4736 | 4647 | 4778 | 78 | 86 | 68 | N |

| | | | | | | | | | | |
|---|---|---|---|---|---|---|---|---|---|---|
| ATOM | 300 | CZ | ARG A | 438 | 25.791 | 36.661 | 63.952 | 1.00 | 38.91 | C |

| | | | | | | | | | | | |
|---|---|---|---|---|---|---|---|---|---|---|---|
| ANISOU | 300 | CZ | ARG A | 438 | 4806 | 4921 | 5057 | −41 | 97 | 76 | C |

| | | | | | | | | | | |
|---|---|---|---|---|---|---|---|---|---|---|
| ATOM | 301 | NH1 | ARG A | 438 | 25.458 | 35.414 | 64.240 | 1.00 | 39.55 | N |

| | | | | | | | | | | | |
|---|---|---|---|---|---|---|---|---|---|---|---|
| ANISOU | 301 | NH1 | ARG A | 438 | 4980 | 4877 | 5171 | −4 | 108 | 66 | N |

| | | | | | | | | | | |
|---|---|---|---|---|---|---|---|---|---|---|
| ATOM | 302 | NH2 | ARG A | 438 | 25.045 | 37.650 | 64.444 | 1.00 | 39.77 | N |

| | | | | | | | | | | | |
|---|---|---|---|---|---|---|---|---|---|---|---|
| ANISOU | 302 | NH2 | ARG A | 438 | 4958 | 4962 | 5191 | −40 | 97 | −21 | N |

| | | | | | | | | | | |
|---|---|---|---|---|---|---|---|---|---|---|
| ATOM | 303 | N | ASP A | 439 | 29.495 | 32.063 | 65.276 | 1.00 | 20.60 | N |

| | | | | | | | | | | | |
|---|---|---|---|---|---|---|---|---|---|---|---|
| ANISOU | 303 | N | ASP A | 439 | 2708 | 2801 | 2319 | −32 | 66 | 39 | N |

| | | | | | | | | | | |
|---|---|---|---|---|---|---|---|---|---|---|
| ATOM | 304 | CA | ASP A | 439 | 28.883 | 31.366 | 66.411 | 1.00 | 21.02 | C |

| | | | | | | | | | | | |
|---|---|---|---|---|---|---|---|---|---|---|---|
| ANISOU | 304 | CA | ASP A | 439 | 2826 | 2641 | 2521 | −82 | 66 | 72 | C |

| | | | | | | | | | | |
|---|---|---|---|---|---|---|---|---|---|---|
| ATOM | 305 | C | ASP A | 439 | 29.881 | 31.095 | 67.540 | 1.00 | 20.11 | C |

| | | | | | | | | | | | |
|---|---|---|---|---|---|---|---|---|---|---|---|
| ANISOU | 305 | C | ASP A | 439 | 2514 | 2517 | 2609 | 4 | 88 | −1 | C |

| | | | | | | | | | | |
|---|---|---|---|---|---|---|---|---|---|---|
| ATOM | 306 | O | ASP A | 439 | 29.634 | 31.277 | 68.721 | 1.00 | 19.64 | O |

| | | | | | | | | | | | |
|---|---|---|---|---|---|---|---|---|---|---|---|
| ANISOU | 306 | O | ASP A | 439 | 2660 | 2230 | 2572 | −3 | 154 | 26 | O |

| | | | | | | | | | | |
|---|---|---|---|---|---|---|---|---|---|---|
| ATOM | 307 | CB | ASP A | 439 | 28.402 | 29.981 | 65.963 | 1.00 | 20.69 | C |

| | | | | | | | | | | | |
|---|---|---|---|---|---|---|---|---|---|---|---|
| ANISOU | 307 | CB | ASP A | 439 | 2678 | 2756 | 2427 | −199 | 128 | −20 | C |

| | | | | | | | | | | |
|---|---|---|---|---|---|---|---|---|---|---|
| ATOM | 308 | CG | ASP A | 439 | 27.382 | 30.072 | 64.866 | 1.00 | 20.88 | C |

| | | | | | | | | | | | |
|---|---|---|---|---|---|---|---|---|---|---|---|
| ANISOU | 308 | CG | ASP A | 439 | 2724 | 2697 | 2511 | −96 | 144 | 102 | C |

| | | | | | | | | | | |
|---|---|---|---|---|---|---|---|---|---|---|
| ATOM | 309 | OD1 | ASP A | 439 | 26.366 | 30.761 | 65.013 | 1.00 | 21.12 | O |

| | | | | | | | | | | | |
|---|---|---|---|---|---|---|---|---|---|---|---|
| ANISOU | 309 | OD1 | ASP A | 439 | 2808 | 2896 | 2320 | 41 | −29 | 6 | O |

| | | | | | | | | | | |
|---|---|---|---|---|---|---|---|---|---|---|
| ATOM | 310 | OD2 | ASP A | 439 | 27.556 | 29.433 | 63.810 | 1.00 | 23.27 | O |

| | | | | | | | | | | | |
|---|---|---|---|---|---|---|---|---|---|---|---|
| ANISOU | 310 | OD2 | ASP A | 439 | 3051 | 3241 | 2551 | −67 | 10 | −162 | O |

| | | | | | | | | | | |
|---|---|---|---|---|---|---|---|---|---|---|
| ATOM | 311 | N | PHE A | 440 | 31.076 | 30.603 | 67.161 | 1.00 | 19.54 | N |

| | | | | | | | | | | | |
|---|---|---|---|---|---|---|---|---|---|---|---|
| ANISOU | 311 | N | PHE A | 440 | 2493 | 2361 | 2571 | 85 | 43 | 78 | N |

| | | | | | | | | | | |
|---|---|---|---|---|---|---|---|---|---|---|
| ATOM | 312 | CA | PHE A | 440 | 32.100 | 30.246 | 68.128 | 1.00 | 19.79 | C |

| | | | | | | | | | | | |
|---|---|---|---|---|---|---|---|---|---|---|---|
| ANISOU | 312 | CA | PHE A | 440 | 2425 | 2592 | 2502 | 26 | 67 | −134 | C |

| | | | | | | | | | | |
|---|---|---|---|---|---|---|---|---|---|---|
| ATOM | 313 | C | PHE A | 440 | 32.518 | 31.417 | 68.988 | 1.00 | 19.93 | C |

| | | | | | | | | | | | |
|---|---|---|---|---|---|---|---|---|---|---|---|
| ANISOU | 313 | C | PHE A | 440 | 2593 | 2616 | 2365 | 10 | −47 | −24 | C |

| | | | | | | | | | | |
|---|---|---|---|---|---|---|---|---|---|---|
| ATOM | 314 | O | PHE A | 440 | 32.755 | 31.232 | 70.186 | 1.00 | 18.54 | O |

| | | | | | | | | | | | |
|---|---|---|---|---|---|---|---|---|---|---|---|
| ANISOU | 314 | O | PHE A | 440 | 2415 | 2488 | 2140 | 3 | 225 | 1 | O |

| | | | | | | | | | | |
|---|---|---|---|---|---|---|---|---|---|---|
| ATOM | 315 | CB | PHE A | 440 | 33.298 | 29.737 | 67.297 | 1.00 | 19.80 | C |

| | | | | | | | | | | | |
|---|---|---|---|---|---|---|---|---|---|---|---|
| ANISOU | 315 | CB | PHE A | 440 | 2304 | 2693 | 2526 | 145 | 190 | −16 | C |

| | | | | | | | | | | |
|---|---|---|---|---|---|---|---|---|---|---|
| ATOM | 316 | CG | PHE A | 440 | 34.412 | 29.109 | 68.102 | 1.00 | 19.76 | C |

| | | | | | | | | | | | |
|---|---|---|---|---|---|---|---|---|---|---|---|
| ANISOU | 316 | CG | PHE A | 440 | 2443 | 2654 | 2409 | 38 | 102 | −108 | C |

| | | | | | | | | | | |
|---|---|---|---|---|---|---|---|---|---|---|
| ATOM | 317 | CD1 | PHE A | 440 | 34.295 | 27.791 | 68.493 | 1.00 | 20.57 | C |

| | | | | | | | | | | | |
|---|---|---|---|---|---|---|---|---|---|---|---|
| ANISOU | 317 | CD1 | PHE A | 440 | 2658 | 2707 | 2451 | 20 | 81 | −146 | C |

| | | | | | | | | | | |
|---|---|---|---|---|---|---|---|---|---|---|
| ATOM | 318 | CD2 | PHE A | 440 | 35.575 | 29.780 | 68.471 | 1.00 | 20.34 | C |

| | | | | | | | | | | | |
|---|---|---|---|---|---|---|---|---|---|---|---|
| ANISOU | 318 | CD2 | PHE A | 440 | 2594 | 2644 | 2492 | −44 | 137 | −151 | C |

| | | | | | | | | | | |
|---|---|---|---|---|---|---|---|---|---|---|
| ATOM | 319 | CE1 | PHE A | 440 | 35.257 | 27.125 | 69.211 | 1.00 | 19.74 | C |

| | | | | | | | | | | | |
|---|---|---|---|---|---|---|---|---|---|---|---|
| ANISOU | 319 | CE1 | PHE A | 440 | 2616 | 2507 | 2375 | 21 | 98 | −183 | C |

| | | | | | | | | | | |
|---|---|---|---|---|---|---|---|---|---|---|
| ATOM | 320 | CE2 | PHE A | 440 | 36.562 | 29.155 | 69.213 | 1.00 | 20.51 | C |

| | | | | | | | | | | | |
|---|---|---|---|---|---|---|---|---|---|---|---|
| ANISOU | 320 | CE2 | PHE A | 440 | 2598 | 2731 | 2464 | 12 | 133 | −146 | C |

| | | | | | | | | | | |
|---|---|---|---|---|---|---|---|---|---|---|
| ATOM | 321 | CZ | PHE A | 440 | 36.390 | 27.834 | 69.569 | 1.00 | 20.48 | C |

| | | | | | | | | | | | |
|---|---|---|---|---|---|---|---|---|---|---|---|
| ANISOU | 321 | CZ | PHE A | 440 | 2429 | 2757 | 2594 | 95 | 43 | −26 | C |

| | | | | | | | | | | |
|---|---|---|---|---|---|---|---|---|---|---|
| ATOM | 322 | N | LYS A | 441 | 32.564 | 32.610 | 68.418 | 1.00 | 19.50 | N |

| | | | | | | | | | | | |
|---|---|---|---|---|---|---|---|---|---|---|---|
| ANISOU | 322 | N | LYS A | 441 | 2444 | 2481 | 2483 | 50 | 21 | −45 | N |

| | | | | | | | | | | |
|---|---|---|---|---|---|---|---|---|---|---|
| ATOM | 323 | CA | LYS A | 441 | 32.969 | 33.788 | 69.187 | 1.00 | 21.01 | C |

| | | | | | | | | | | | |
|---|---|---|---|---|---|---|---|---|---|---|---|
| ANISOU | 323 | CA | LYS A | 441 | 2695 | 2660 | 2628 | −30 | 16 | −109 | C |

| | | | | | | | | | | |
|---|---|---|---|---|---|---|---|---|---|---|
| ATOM | 324 | C | LYS A | 441 | 31.985 | 34.146 | 70.277 | 1.00 | 20.16 | C |

| | | | | | | | | | | | |
|---|---|---|---|---|---|---|---|---|---|---|---|
| ANISOU | 324 | C | LYS A | 441 | 2600 | 2481 | 2578 | −113 | 34 | −23 | C |

| | | | | | | | | | | |
|---|---|---|---|---|---|---|---|---|---|---|
| ATOM | 325 | O | LYS A | 441 | 32.310 | 34.937 | 71.190 | 1.00 | 20.20 | O |

| | | | | | | | | | | | |
|---|---|---|---|---|---|---|---|---|---|---|---|
| ANISOU | 325 | O | LYS A | 441 | 2843 | 2418 | 2416 | −5 | −152 | 166 | O |

| | | | | | | | | | | |
|---|---|---|---|---|---|---|---|---|---|---|
| ATOM | 326 | CB | LYS A | 441 | 33.190 | 34.975 | 68.233 | 1.00 | 23.03 | C |

| | | | | | | | | | | | |
|---|---|---|---|---|---|---|---|---|---|---|---|
| ANISOU | 326 | CB | LYS A | 441 | 3250 | 2780 | 2721 | −106 | 9 | −41 | C |

| | | | | | | | | | | |
|---|---|---|---|---|---|---|---|---|---|---|
| ATOM | 327 | CG | LYS A | 441 | 34.500 | 34.788 | 67.425 | 1.00 | 26.36 | C |

| | | | | | | | | | | | |
|---|---|---|---|---|---|---|---|---|---|---|---|
| ANISOU | 327 | CG | LYS A | 441 | 3129 | 3556 | 3331 | −82 | 24 | −118 | C |

| | | | | | | | | | | |
|---|---|---|---|---|---|---|---|---|---|---|
| ATOM | 328 | CD | LYS A | 441 | 34.588 | 36.012 | 66.540 | 1.00 | 30.45 | C |

| | | | | | | | | | | | |
|---|---|---|---|---|---|---|---|---|---|---|---|
| ANISOU | 328 | CD | LYS A | 441 | 4080 | 3816 | 3675 | −46 | −53 | 102 | C |

| | | | | | | | | | | |
|---|---|---|---|---|---|---|---|---|---|---|
| ATOM | 329 | CE | LYS A | 441 | 35.842 | 36.256 | 65.744 | 1.00 | 33.52 | C |

| | | | | | | | | | | | |
|---|---|---|---|---|---|---|---|---|---|---|---|
| ANISOU | 329 | CE | LYS A | 441 | 4141 | 4332 | 4262 | 19 | 58 | 49 | C |

| | | | | | | | | | | |
|---|---|---|---|---|---|---|---|---|---|---|
| ATOM | 330 | NZ | LYS A | 441 | 35.868 | 37.745 | 65.450 | 1.00 | 35.91 | N |

| | | | | | | | | | | | |
|---|---|---|---|---|---|---|---|---|---|---|---|
| ANISOU | 330 | NZ | LYS A | 441 | 4740 | 4423 | 4480 | −115 | 13 | 200 | N |

| | | | | | | | | | | |
|---|---|---|---|---|---|---|---|---|---|---|
| ATOM | 331 | N | THR A | 442 | 30.747 | 33.575 | 70.269 | 1.00 | 18.17 | N |

| | | | | | | | | | | | |
|---|---|---|---|---|---|---|---|---|---|---|---|
| ANISOU | 331 | N | THR A | 442 | 2284 | 2270 | 2351 | 137 | 149 | −65 | N |

| | | | | | | | | | | |
|---|---|---|---|---|---|---|---|---|---|---|
| ATOM | 332 | CA | THR A | 442 | 29.833 | 33.861 | 71.383 | 1.00 | 17.18 | C |

| | | | | | | | | | | | |
|---|---|---|---|---|---|---|---|---|---|---|---|
| ANISOU | 332 | CA | THR A | 442 | 2409 | 1884 | 2237 | 18 | 106 | −101 | C |

| | | | | | | | | | | |
|---|---|---|---|---|---|---|---|---|---|---|
| ATOM | 333 | C | THR A | 442 | 30.334 | 33.146 | 72.834 | 1.00 | 16.91 | C |

| | | | | | | | | | | | |
|---|---|---|---|---|---|---|---|---|---|---|---|
| ANISOU | 333 | C | THR A | 442 | 2248 | 1942 | 2236 | −42 | 130 | −14 | C |

| | | | | | | | | | | |
|---|---|---|---|---|---|---|---|---|---|---|
| ATOM | 334 | O | THR A | 442 | 28.800 | 33.382 | 73.738 | 1.00 | 17.17 | O |

| | | | | | | | | | | | |
|---|---|---|---|---|---|---|---|---|---|---|---|
| ANISOU | 334 | O | THR A | 442 | 2627 | 1783 | 2114 | −60 | 151 | 53 | O |

| | | | | | | | | | | |
|---|---|---|---|---|---|---|---|---|---|---|
| ATOM | 335 | CB | THR A | 442 | 28.360 | 33.521 | 71.114 | 1.00 | 16.82 | C |

| | | | | | | | | | | | |
|---|---|---|---|---|---|---|---|---|---|---|---|
| ANISOU | 335 | CB | THR A | 442 | 2340 | 1792 | 2258 | 59 | 8 | 6 | C |

| | | | | | | | | | | |
|---|---|---|---|---|---|---|---|---|---|---|
| ATOM | 336 | OG1 | THR A | 442 | 28.140 | 32.089 | 71.013 | 1.00 | 17.59 | O |

| | | | | | | | | | | | |
|---|---|---|---|---|---|---|---|---|---|---|---|
| ANISOU | 336 | OG1 | THR A | 442 | 2466 | 1836 | 2382 | −53 | 121 | 189 | O |

| | | | | | | | | | | |
|---|---|---|---|---|---|---|---|---|---|---|
| ATOM | 337 | CG2 | THR A | 442 | 27.887 | 34.234 | 69.832 | 1.00 | 18.39 | C |

| | | | | | | | | | | | |
|---|---|---|---|---|---|---|---|---|---|---|---|
| ANISOU | 337 | CG2 | THR A | 442 | 2507 | 2362 | 2116 | −31 | 117 | 96 | C |

| | | | | | | | | | | |
|---|---|---|---|---|---|---|---|---|---|---|
| ATOM | 338 | N | LEU A | 443 | 31.319 | 32.255 | 72.548 | 1.00 | 16.60 | N |

| | | | | | | | | | | | |
|---|---|---|---|---|---|---|---|---|---|---|---|
| ANISOU | 338 | N | LEU A | 443 | 2351 | 1803 | 2154 | −60 | 30 | −2 | N |

| | | | | | | | | | | |
|---|---|---|---|---|---|---|---|---|---|---|
| ATOM | 339 | CA | LEU A | 443 | 31.910 | 31.625 | 73.715 | 1.00 | 17.07 | C |

| | | | | | | | | | | | |
|---|---|---|---|---|---|---|---|---|---|---|---|
| ANISOU | 339 | CA | LEU A | 443 | 2333 | 1914 | 2240 | −32 | −87 | −1 | C |

| | | | | | | | | | | |
|---|---|---|---|---|---|---|---|---|---|---|
| ATOM | 340 | C | LEU A | 443 | 33.094 | 32.445 | 74.273 | 1.00 | 17.58 | C |

| | | | | | | | | | | | |
|---|---|---|---|---|---|---|---|---|---|---|---|
| ANISOU | 340 | C | LEU A | 443 | 2376 | 2064 | 2239 | −107 | −4 | −12 | C |

| | | | | | | | | | | |
|---|---|---|---|---|---|---|---|---|---|---|
| ATOM | 341 | O | LEU A | 443 | 33.661 | 32.059 | 75.292 | 1.00 | 17.55 | O |

| | | | | | | | | | | | |
|---|---|---|---|---|---|---|---|---|---|---|---|
| ANISOU | 341 | O | LEU A | 443 | 2259 | 2037 | 2371 | −152 | −66 | −82 | O |

| | | | | | | | | | | |
|---|---|---|---|---|---|---|---|---|---|---|
| ATOM | 342 | CB | LEU A | 443 | 32.463 | 30.205 | 73.358 | 1.00 | 16.65 | C |

| | | | | | | | | | | | |
|---|---|---|---|---|---|---|---|---|---|---|---|
| ANISOU | 342 | CB | LEU A | 443 | 2336 | 1860 | 2131 | −40 | −109 | 52 | C |

| | | | | | | | | | | |
|---|---|---|---|---|---|---|---|---|---|---|
| ATOM | 343 | CG | LEU A | 443 | 31.329 | 29.177 | 73.206 | 1.00 | 15.42 | C |

| | | | | | | | | | | | |
|---|---|---|---|---|---|---|---|---|---|---|---|
| ANISOU | 343 | CG | LEU A | 443 | 2151 | 1866 | 1839 | 74 | −50 | −14 | C |

| | | | | | | | | | | |
|---|---|---|---|---|---|---|---|---|---|---|
| ATOM | 344 | CD1 | LEU A | 443 | 31.840 | 27.988 | 72.401 | 1.00 | 16.51 | C |

| | | | | | | | | | | | |
|---|---|---|---|---|---|---|---|---|---|---|---|
| ANISOU | 344 | CD1 | LEU A | 443 | 2345 | 1676 | 2250 | −2 | −58 | −104 | C |

| | | | | | | | | | | |
|---|---|---|---|---|---|---|---|---|---|---|
| ATOM | 345 | CD2 | LEU A | 443 | 30.799 | 28.717 | 74.570 | 1.00 | 17.06 | C |

| | | | | | | | | | | | |
|---|---|---|---|---|---|---|---|---|---|---|---|
| ANISOU | 345 | CD2 | LEU A | 443 | 2415 | 2020 | 2046 | −251 | 29 | 65 | C |

| | | | | | | | | | | |
|---|---|---|---|---|---|---|---|---|---|---|
| ATOM | 346 | N | ALA A | 444 | 33.470 | 33.527 | 73.598 | 1.00 | 17.62 | N |

| | | | | | | | | | | | |
|---|---|---|---|---|---|---|---|---|---|---|---|
| ANISOU | 346 | N | ALA A | 444 | 2326 | 1928 | 2440 | −68 | 21 | −85 | N |

| | | | | | | | | | | |
|---|---|---|---|---|---|---|---|---|---|---|
| ATOM | 347 | CA | ALA A | 444 | 34.556 | 34.360 | 74.105 | 1.00 | 17.07 | C |

| | | | | | | | | | | | |
|---|---|---|---|---|---|---|---|---|---|---|---|
| ANISOU | 347 | CA | ALA A | 444 | 2325 | 1896 | 2263 | −9 | O | −52 | C |

| | | | | | | | | | | |
|---|---|---|---|---|---|---|---|---|---|---|
| ATOM | 348 | C | ALA A | 444 | 34.158 | 34.904 | 75.463 | 1.00 | 17.32 | C |

| | | | | | | | | | | | |
|---|---|---|---|---|---|---|---|---|---|---|---|
| ANISOU | 348 | C | ALA A | 444 | 2281 | 1987 | 2313 | −64 | −11 | −174 | C |

| | | | | | | | | | | |
|---|---|---|---|---|---|---|---|---|---|---|
| ATOM | 349 | O | ALA A | 444 | 32.971 | 34.984 | 75.854 | 1.00 | 16.91 | O |

| | | | | | | | | | | | |
|---|---|---|---|---|---|---|---|---|---|---|---|
| ANISOU | 349 | O | ALA A | 444 | 2223 | 1714 | 2488 | −248 | 67 | −154 | O |

| | | | | | | | | | | |
|---|---|---|---|---|---|---|---|---|---|---|
| ATOM | 350 | CB | ALA A | 444 | 34.842 | 35.452 | 73.087 | 1.00 | 17.28 | C |

| | | | | | | | | | | | |
|---|---|---|---|---|---|---|---|---|---|---|---|
| ANISOU | 350 | CB | ALA A | 444 | 2179 | 2112 | 2272 | −272 | 109 | −37 | C |

| | | | | | | | | | | |
|---|---|---|---|---|---|---|---|---|---|---|
| ATOM | 351 | N | PRO A | 445 | 35.137 | 35.368 | 76.250 | 1.00 | 16.22 | N |

| | | | | | | | | | | | |
|---|---|---|---|---|---|---|---|---|---|---|---|
| ANISOU | 351 | N | PRO A | 445 | 2052 | 1897 | 2214 | −188 | 107 | −128 | N |

| | | | | | | | | | | |
|---|---|---|---|---|---|---|---|---|---|---|
| ATOM | 352 | CA | PRO A | 445 | 34.786 | 35.874 | 77.559 | 1.00 | 15.65 | C |

| | | | | | | | | | | | |
|---|---|---|---|---|---|---|---|---|---|---|---|
| ANISOU | 352 | CA | PRO A | 445 | 1914 | 1784 | 2246 | −142 | 45 | −105 | C |

| | | | | | | | | | | |
|---|---|---|---|---|---|---|---|---|---|---|
| ATOM | 353 | C | PRO A | 445 | 33.720 | 36.952 | 77.505 | 1.00 | 16.92 | C |

| | | | | | | | | | | | |
|---|---|---|---|---|---|---|---|---|---|---|---|
| ANISOU | 353 | C | PRO A | 445 | 2006 | 2087 | 2335 | −85 | 70 | −74 | C |

| | | | | | | | | | | |
|---|---|---|---|---|---|---|---|---|---|---|
| ATOM | 354 | O | PRO A | 445 | 33.726 | 37.891 | 76.691 | 1.00 | 17.87 | O |

| | | | | | | | | | | | |
|---|---|---|---|---|---|---|---|---|---|---|---|
| ANISOU | 354 | O | PRO A | 445 | 2256 | 1821 | 2715 | −64 | 76 | −100 | O |

| | | | | | | | | | | |
|---|---|---|---|---|---|---|---|---|---|---|
| ATOM | 355 | CB | PRO A | 445 | 36.135 | 36.426 | 78.076 | 1.00 | 18.07 | C |

| | | | | | | | | | | | |
|---|---|---|---|---|---|---|---|---|---|---|---|
| ANISOU | 355 | CB | PRO A | 445 | 1973 | 2091 | 2802 | −181 | −144 | −249 | C |

| | | | | | | | | | | |
|---|---|---|---|---|---|---|---|---|---|---|
| ATOM | 356 | CG | PRO A | 445 | 37.154 | 35.585 | 77.372 | 1.00 | 20.04 | C |

| | | | | | | | | | | | |
|---|---|---|---|---|---|---|---|---|---|---|---|
| ANISOU | 356 | CG | PRO A | 445 | 2378 | 2661 | 2575 | 15 | −1 | −296 | C |

| | | | | | | | | | | |
|---|---|---|---|---|---|---|---|---|---|---|
| ATOM | 357 | CD | PRO A | 445 | 36.592 | 35.356 | 75.986 | 1.00 | 19.05 | C |

| | | | | | | | | | | | |
|---|---|---|---|---|---|---|---|---|---|---|---|
| ANISOU | 357 | CD | PRO A | 445 | 2010 | 2515 | 2712 | −155 | −149 | −187 | C |

| | | | | | | | | | | |
|---|---|---|---|---|---|---|---|---|---|---|
| ATOM | 358 | N | ARG A | 446 | 32.799 | 36.895 | 78.445 | 1.00 | 16.51 | N |

| | | | | | | | | | | | |
|---|---|---|---|---|---|---|---|---|---|---|---|
| ANISOU | 358 | N | ARG A | 446 | 1952 | 1954 | 2367 | −169 | 51 | −34 | N |

| | | | | | | | | | | |
|---|---|---|---|---|---|---|---|---|---|---|
| ATOM | 359 | CA | ARG A | 446 | 31.738 | 37.892 | 78.659 | 1.00 | 16.52 | C |

| | | | | | | | | | | | |
|---|---|---|---|---|---|---|---|---|---|---|---|
| ANISOU | 359 | CA | ARG A | 446 | 2082 | 1884 | 2312 | −18 | 31 | 3 | C |

| | | | | | | | | | | |
|---|---|---|---|---|---|---|---|---|---|---|
| ATOM | 360 | C | ARG A | 446 | 30.707 | 37.967 | 77.542 | 1.00 | 18.18 | C |

| | | | | | | | | | | | |
|---|---|---|---|---|---|---|---|---|---|---|---|
| ANISOU | 360 | C | ARG A | 446 | 2455 | 2151 | 2302 | 3 | −50 | −38 | C |

| | | | | | | | | | | |
|---|---|---|---|---|---|---|---|---|---|---|
| ATOM | 361 | O | ARG A | 446 | 29.800 | 38.854 | 77.560 | 1.00 | 19.83 | O |

| | | | | | | | | | | | |
|---|---|---|---|---|---|---|---|---|---|---|---|
| ANISOU | 361 | O | ARG A | 446 | 2447 | 2435 | 2653 | 322 | 95 | −144 | O |

| | | | | | | | | | | |
|---|---|---|---|---|---|---|---|---|---|---|
| ATOM | 362 | CB | ARG A | 446 | 32.354 | 39.302 | 78.904 | 1.00 | 18.67 | C |

| | | | | | | | | | | | |
|---|---|---|---|---|---|---|---|---|---|---|---|
| ANISOU | 362 | CB | ARG A | 446 | 2584 | 1873 | 2638 | −46 | −139 | −11 | C |

| | | | | | | | | | | |
|---|---|---|---|---|---|---|---|---|---|---|
| ATOM | 363 | CG | ARG A | 446 | 33.233 | 39.352 | 80.164 | 1.00 | 19.18 | C |

| | | | | | | | | | | | |
|---|---|---|---|---|---|---|---|---|---|---|---|
| ANISOU | 363 | CG | ARG A | 446 | 2624 | 2013 | 2649 | −256 | −95 | 136 | C |

| | | | | | | | | | | |
|---|---|---|---|---|---|---|---|---|---|---|
| ATOM | 364 | CD | ARG A | 446 | 33.825 | 40.761 | 80.413 | 1.00 | 21.48 | C |

| | | | | | | | | | | | |
|---|---|---|---|---|---|---|---|---|---|---|---|
| ANISOU | 364 | CD | ARG A | 446 | 3307 | 1870 | 2983 | −217 | 112 | −27 | C |

| | | | | | | | | | | |
|---|---|---|---|---|---|---|---|---|---|---|
| ATOM | 365 | NE | ARG A | 446 | 34.445 | 40.745 | 81.772 | 1.00 | 24.17 | N |

| | | | | | | | | | | | |
|---|---|---|---|---|---|---|---|---|---|---|---|
| ANISOU | 365 | NE | ARG A | 446 | 3506 | 2312 | 3366 | −393 | −36 | −98 | N |

| | | | | | | | | | | |
|---|---|---|---|---|---|---|---|---|---|---|
| ATOM | 366 | CZ | ARG A | 446 | 33.579 | 41.237 | 82.720 | 1.00 | 26.50 | C |

| | | | | | | | | | | | |
|---|---|---|---|---|---|---|---|---|---|---|---|
| ANISOU | 366 | CZ | ARG A | 446 | 3650 | 2911 | 3507 | −271 | 109 | −165 | C |

| | | | | | | | | | | |
|---|---|---|---|---|---|---|---|---|---|---|
| ATOM | 367 | NH1 | ARG A | 446 | 32.341 | 41.809 | 82.594 | 1.00 | 27.49 | N |

| | | | | | | | | | | | |
|---|---|---|---|---|---|---|---|---|---|---|---|
| ANISOU | 367 | NH1 | ARG A | 446 | 3540 | 3097 | 3809 | −527 | 31 | −60 | N |

| | | | | | | | | | | |
|---|---|---|---|---|---|---|---|---|---|---|
| ATOM | 368 | NH2 | ARG A | 446 | 34.141 | 41.150 | 83.892 | 1.00 | 27.19 | N |

| | | | | | | | | | | | |
|---|---|---|---|---|---|---|---|---|---|---|---|
| ANISOU | 368 | NH2 | ARG A | 446 | 3631 | 3046 | 3654 | −92 | 132 | 179 | N |

| | | | | | | | | | | |
|---|---|---|---|---|---|---|---|---|---|---|
| ATOM | 369 | N | ARG A | 447 | 30.739 | 37.078 | 76.546 | 1.00 | 17.24 | N |

| | | | | | | | | | | | |
|---|---|---|---|---|---|---|---|---|---|---|---|
| ANISOU | 369 | N | ARG A | 447 | 2395 | 1882 | 2274 | −133 | 42 | 56 | N |

| | | | | | | | | | | |
|---|---|---|---|---|---|---|---|---|---|---|
| ATOM | 370 | CA | ARG A | 447 | 29.759 | 37.166 | 75.475 | 1.00 | 17.00 | C |

| | | | | | | | | | | | |
|---|---|---|---|---|---|---|---|---|---|---|---|
| ANISOU | 370 | CA | ARG A | 447 | 2288 | 1975 | 2197 | 20 | 89 | 44 | C |

| | | | | | | | | | | |
|---|---|---|---|---|---|---|---|---|---|---|
| ATOM | 371 | C | ARG A | 447 | 28.527 | 36.318 | 75.630 | 1.00 | 17.43 | C |

| | | | | | | | | | | | |
|---|---|---|---|---|---|---|---|---|---|---|---|
| ANISOU | 371 | C | ARG A | 447 | 2509 | 1923 | 2191 | −78 | −31 | 138 | C |

| | | | | | | | | | | |
|---|---|---|---|---|---|---|---|---|---|---|
| ATOM | 372 | O | ARG A | 447 | 28.527 | 35.277 | 76.306 | 1.00 | 17.13 | O |

| | | | | | | | | | | | |
|---|---|---|---|---|---|---|---|---|---|---|---|
| ANISOU | 372 | O | ARG A | 447 | 2492 | 1791 | 2225 | −80 | 98 | 190 | O |

| | | | | | | | | | | |
|---|---|---|---|---|---|---|---|---|---|---|
| ATOM | 373 | CB | ARG A | 447 | 30.422 | 36.761 | 74.134 | 1.00 | 16.08 | C |

| | | | | | | | | | | | |
|---|---|---|---|---|---|---|---|---|---|---|---|
| ANISOU | 373 | CB | ARG A | 447 | 2170 | 1875 | 2065 | 1 | 67 | 88 | C |

| | | | | | | | | | | |
|---|---|---|---|---|---|---|---|---|---|---|
| ATOM | 374 | CG | ARG A | 447 | 31.620 | 37.628 | 73.746 | 1.00 | 19.23 | C |

| | | | | | | | | | | | |
|---|---|---|---|---|---|---|---|---|---|---|---|
| ANISOU | 374 | CG | ARG A | 447 | 2503 | 2150 | 2653 | −127 | 85 | 139 | C |

| | | | | | | | | | | |
|---|---|---|---|---|---|---|---|---|---|---|
| ATOM | 375 | CD | ARG A | 447 | 31.181 | 39.084 | 73.539 | 1.00 | 20.07 | C |

| | | | | | | | | | | | |
|---|---|---|---|---|---|---|---|---|---|---|---|
| ANISOU | 375 | CD | ARG A | 447 | 2568 | 2248 | 2811 | −40 | 103 | 217 | C |

| | | | | | | | | | | |
|---|---|---|---|---|---|---|---|---|---|---|
| ATOM | 376 | NE | ARG A | 447 | 32.400 | 39.818 | 73.119 | 1.00 | 21.14 | N |

| | | | | | | | | | | | |
|---|---|---|---|---|---|---|---|---|---|---|---|
| ANISOU | 376 | NE | ARG A | 447 | 2589 | 2668 | 2774 | −259 | 61 | −3 | N |

| | | | | | | | | | | |
|---|---|---|---|---|---|---|---|---|---|---|
| ATOM | 377 | CZ | ARG A | 447 | 32.351 | 41.098 | 72.718 | 1.00 | 22.82 | C |

| | | | | | | | | | | | |
|---|---|---|---|---|---|---|---|---|---|---|---|
| ANISOU | 377 | CZ | ARG A | 447 | 3041 | 2717 | 2911 | −130 | 36 | 41 | C |

| | | | | | | | | | | |
|---|---|---|---|---|---|---|---|---|---|---|
| ATOM | 378 | NH1 | ARG A | 447 | 31.207 | 41.771 | 72.687 | 1.00 | 23.52 | N |

| | | | | | | | | | | | |
|---|---|---|---|---|---|---|---|---|---|---|---|
| ANISOU | 378 | NH1 | ARG A | 447 | 3130 | 2784 | 3022 | −93 | 124 | 37 | N |

| | | | | | | | | | | |
|---|---|---|---|---|---|---|---|---|---|---|
| ATOM | 379 | NH2 | ARG A | 447 | 33.497 | 41.683 | 72.324 | 1.00 | 23.53 | N |

| | | | | | | | | | | | |
|---|---|---|---|---|---|---|---|---|---|---|---|
| ANISOU | 379 | NH2 | ARG A | 447 | 2965 | 2843 | 3133 | −91 | 7 | −139 | N |

| | | | | | | | | | | |
|---|---|---|---|---|---|---|---|---|---|---|
| ATOM | 380 | N | TRP A | 448 | 27.415 | 36.853 | 75.135 | 1.00 | 16.90 | N |

| | | | | | | | | | | | |
|---|---|---|---|---|---|---|---|---|---|---|---|
| ANISOU | 380 | N | TRP A | 448 | 2153 | 1985 | 2283 | −4 | 82 | −5 | N |

| | | | | | | | | | | |
|---|---|---|---|---|---|---|---|---|---|---|
| ATOM | 381 | CA | TRP A | 448 | 26.139 | 36.105 | 75.144 | 1.00 | 16.65 | C |

| | | | | | | | | | | | |
|---|---|---|---|---|---|---|---|---|---|---|---|
| ANISOU | 381 | CA | TRP A | 448 | 2251 | 2000 | 2075 | 13 | −11 | 111 | C |

| | | | | | | | | | | |
|---|---|---|---|---|---|---|---|---|---|---|
| ATOM | 382 | C | TRR A | 448 | 26.161 | 34.924 | 74.171 | 1.00 | 17.62 | C |

| | | | | | | | | | | | |
|---|---|---|---|---|---|---|---|---|---|---|---|
| ANISOU | 382 | C | TRP A | 448 | 2375 | 1998 | 2321 | −5 | 25 | 47 | C |

| | | | | | | | | | | |
|---|---|---|---|---|---|---|---|---|---|---|
| ATOM | 383 | O | TRP A | 448 | 26.341 | 35.113 | 72.966 | 1.00 | 18.85 | O |

| | | | | | | | | | | | |
|---|---|---|---|---|---|---|---|---|---|---|---|
| ANISOU | 383 | O | TRP A | 448 | 2476 | 2141 | 2544 | 197 | 299 | 108 | O |

| | | | | | | | | | | |
|---|---|---|---|---|---|---|---|---|---|---|
| ATOM | 384 | CB | TRP A | 448 | 25.059 | 37.124 | 74.759 | 1.00 | 16.46 | C |

| | | | | | | | | | | | |
|---|---|---|---|---|---|---|---|---|---|---|---|
| ANISOU | 384 | CB | TRP A | 448 | 2329 | 1818 | 2108 | 75 | 127 | 68 | C |

| | | | | | | | | | | |
|---|---|---|---|---|---|---|---|---|---|---|
| ATOM | 385 | CG | TRP A | 448 | 24.692 | 38.108 | 75.831 | 1.00 | 17.23 | C |

| | | | | | | | | | | | |
|---|---|---|---|---|---|---|---|---|---|---|---|
| ANISOU | 385 | CG | TRP A | 448 | 2464 | 1978 | 2105 | −35 | 66 | 36 | C |

| | | | | | | | | | | |
|---|---|---|---|---|---|---|---|---|---|---|
| ATOM | 386 | CD1 | TRP A | 448 | 25.421 | 38.548 | 76.901 | 1.00 | 16.52 | C |

| | | | | | | | | | | | |
|---|---|---|---|---|---|---|---|---|---|---|---|
| ANISOU | 386 | CD1 | TRP A | 448 | 2378 | 1796 | 2114 | 14 | 48 | 126 | C |

| | | | | | | | | | | |
|---|---|---|---|---|---|---|---|---|---|---|
| ATOM | 387 | CD2 | TRP A | 448 | 23.428 | 38.829 | 75.843 | 1.00 | 16.62 | C |

| | | | | | | | | | | | |
|---|---|---|---|---|---|---|---|---|---|---|---|
| ANISOU | 387 | CD2 | TRP A | 448 | 2343 | 1782 | 2192 | −25 | 148 | 214 | C |

| | | | | | | | | | | |
|---|---|---|---|---|---|---|---|---|---|---|
| ATOM | 388 | NE1 | TRP A | 448 | 24.664 | 39.464 | 77.618 | 1.00 | 17.52 | N |

| | | | | | | | | | | | |
|---|---|---|---|---|---|---|---|---|---|---|---|
| ANISOU | 388 | NE1 | TRP A | 448 | 2334 | 2051 | 2272 | 58 | 274 | 166 | N |

| | | | | | | | | | | |
|---|---|---|---|---|---|---|---|---|---|---|
| ATOM | 389 | CE2 | TRP A | 448 | 23.462 | 39.655 | 76.975 | 1.00 | 17.28 | C |

| | | | | | | | | | | | |
|---|---|---|---|---|---|---|---|---|---|---|---|
| ANISOU | 389 | CE2 | TRP A | 448 | 2356 | 1877 | 2332 | 38 | 201 | 121 | C |

| | | | | | | | | | | |
|---|---|---|---|---|---|---|---|---|---|---|
| ATOM | 390 | CE3 | TRP A | 448 | 22.302 | 38.782 | 75.006 | 1.00 | 17.96 | C |

| | | | | | | | | | | | |
|---|---|---|---|---|---|---|---|---|---|---|---|
| ANISOU | 390 | CE3 | TRP A | 448 | 2376 | 1876 | 2572 | −7 | 122 | 291 | C |

| | | | | | | | | | | |
|---|---|---|---|---|---|---|---|---|---|---|
| ATOM | 391 | CZ2 | TRP A | 448 | 22.379 | 40.488 | 77.337 | 1.00 | 17.68 | C |

| | | | | | | | | | | | |
|---|---|---|---|---|---|---|---|---|---|---|---|
| ANISOU | 391 | CZ2 | TRP A | 448 | 2372 | 2030 | 2315 | 84 | 284 | 81 | C |

| | | | | | | | | | | |
|---|---|---|---|---|---|---|---|---|---|---|
| ATOM | 392 | CZ3 | TRP A | 448 | 21.223 | 39.618 | 75.358 | 1.00 | 19.21 | C |

| | | | | | | | | | | | |
|---|---|---|---|---|---|---|---|---|---|---|---|
| ANISOU | 392 | CZ3 | TRP A | 448 | 2588 | 2177 | 2535 | 154 | 134 | 174 | C |

| | | | | | | | | | | |
|---|---|---|---|---|---|---|---|---|---|---|
| ATOM | 393 | CH2 | TRP A | 448 | 21.295 | 40.416 | 76.504 | 1.00 | 19.95 | C |

| | | | | | | | | | | | |
|---|---|---|---|---|---|---|---|---|---|---|---|
| ANISOU | 393 | CH2 | TRP A | 448 | 2647 | 2252 | 2683 | 117 | 76 | 45 | C |

| | | | | | | | | | | |
|---|---|---|---|---|---|---|---|---|---|---|
| ATOM | 394 | N | LEU A | 449 | 25.823 | 33.707 | 74.664 | 1.00 | 16.41 | N |

| | | | | | | | | | | | |
|---|---|---|---|---|---|---|---|---|---|---|---|
| ANISOU | 394 | N | LEU A | 449 | 2220 | 1911 | 2103 | 31 | 72 | 2 | N |

| | | | | | | | | | | |
|---|---|---|---|---|---|---|---|---|---|---|
| ATOM | 395 | CA | LEU A | 449 | 25.751 | 32.578 | 73.733 | 1.00 | 16.20 | C |

| | | | | | | | | | | | |
|---|---|---|---|---|---|---|---|---|---|---|---|
| ANISOU | 395 | CA | LEU A | 449 | 2162 | 1714 | 2279 | −26 | 51 | 77 | C |

| | | | | | | | | | | |
|---|---|---|---|---|---|---|---|---|---|---|
| ATOM | 396 | C | LEU A | 449 | 24.570 | 32.754 | 72.780 | 1.00 | 16.48 | C |

| | | | | | | | | | | | |
|---|---|---|---|---|---|---|---|---|---|---|---|
| ANISOU | 396 | C | LEU A | 449 | 2348 | 1821 | 2094 | 9 | 65 | 193 | C |

| | | | | | | | | | | |
|---|---|---|---|---|---|---|---|---|---|---|
| ATOM | 397 | O | LEU A | 449 | 23.498 | 33.282 | 73.158 | 1.00 | 16.82 | O |

| | | | | | | | | | | | |
|---|---|---|---|---|---|---|---|---|---|---|---|
| ANISOU | 397 | O | LEU A | 449 | 2357 | 1897 | 2138 | 9 | 191 | 253 | O |

| | | | | | | | | | | |
|---|---|---|---|---|---|---|---|---|---|---|
| ATOM | 398 | CB | LEU A | 449 | 25.499 | 31.216 | 74.415 | 1.00 | 18.25 | C |

| | | | | | | | | | | | |
|---|---|---|---|---|---|---|---|---|---|---|---|
| ANISOU | 398 | CB | LEU A | 449 | 2484 | 1985 | 2467 | 45 | 26 | 309 | C |

| | | | | | | | | | | |
|---|---|---|---|---|---|---|---|---|---|---|
| ATOM | 399 | CG | LEU A | 449 | 26.444 | 30.771 | 75.511 | 1.00 | 20.89 | C |

| | | | | | | | | | | | |
|---|---|---|---|---|---|---|---|---|---|---|---|
| ANISOU | 399 | CG | LEU A | 449 | 2977 | 2293 | 2668 | 12 | −206 | 286 | C |

| | | | | | | | | | | |
|---|---|---|---|---|---|---|---|---|---|---|
| ATOM | 400 | CD1 | LEU A | 449 | 26.437 | 29.257 | 75.726 | 1.00 | 19.38 | C |

| | | | | | | | | | | | |
|---|---|---|---|---|---|---|---|---|---|---|---|
| ANISOU | 400 | CD1 | LEU A | 449 | 2722 | 2166 | 2474 | 124 | 189 | 256 | C |

| | | | | | | | | | | |
|---|---|---|---|---|---|---|---|---|---|---|
| ATOM | 401 | CD2 | LEU A | 449 | 27.770 | 31.407 | 75.583 | 1.00 | 20.64 | C |

| | | | | | | | | | | | |
|---|---|---|---|---|---|---|---|---|---|---|---|
| ANISOU | 401 | CD2 | LEU A | 449 | 2591 | 2473 | 2777 | 287 | 4 | 525 | C |

| | | | | | | | | | | |
|---|---|---|---|---|---|---|---|---|---|---|
| ATOM | 402 | N | ASN A | 450 | 24.755 | 32.215 | 71.570 | 1.00 | 16.37 | N |

| | | | | | | | | | | | |
|---|---|---|---|---|---|---|---|---|---|---|---|
| ANISOU | 402 | N | ASN A | 450 | 2335 | 1977 | 1909 | −97 | −33 | 213 | N |

| | | | | | | | | | | |
|---|---|---|---|---|---|---|---|---|---|---|
| ATOM | 403 | CA | ASN A | 450 | 23.617 | 32.195 | 70.626 | 1.00 | 16.97 | C |

| | | | | | | | | | | | |
|---|---|---|---|---|---|---|---|---|---|---|---|
| ANISOU | 403 | CA | ASN A | 450 | 2214 | 2144 | 2090 | −234 | 2 | 149 | C |

| | | | | | | | | | | |
|---|---|---|---|---|---|---|---|---|---|---|
| ATOM | 404 | C | ASN A | 450 | 23.064 | 30.771 | 70.543 | 1.00 | 17.29 | C |

| | | | | | | | | | | | |
|---|---|---|---|---|---|---|---|---|---|---|---|
| ANISOU | 404 | C | ASN A | 450 | 2329 | 1957 | 2283 | 3 | −42 | 70 | C |

| | | | | | | | | | | |
|---|---|---|---|---|---|---|---|---|---|---|
| ATOM | 405 | O | ASN A | 450 | 23.514 | 29.804 | 71.181 | 1.00 | 17.29 | O |

| | | | | | | | | | | | |
|---|---|---|---|---|---|---|---|---|---|---|---|
| ANISOU | 405 | O | ASN A | 450 | 2425 | 2156 | 1988 | −210 | 103 | 340 | O |

| | | | | | | | | | | |
|---|---|---|---|---|---|---|---|---|---|---|
| ATOM | 406 | CB | ASN A | 450 | 23.975 | 32.743 | 69.256 | 1.00 | 18.47 | C |

| | | | | | | | | | | | |
|---|---|---|---|---|---|---|---|---|---|---|---|
| ANISOU | 406 | CB | ASN A | 450 | 2545 | 2410 | 2063 | −22 | 114 | 146 | C |

| | | | | | | | | | | |
|---|---|---|---|---|---|---|---|---|---|---|
| ATOM | 407 | CG | ASN A | 450 | 24.845 | 31.821 | 68.444 | 1.00 | 18.73 | C |

| | | | | | | | | | | | |
|---|---|---|---|---|---|---|---|---|---|---|---|
| ANISOU | 407 | CG | ASN A | 450 | 2422 | 2412 | 2281 | −13 | 254 | 238 | C |

| | | | | | | | | | | |
|---|---|---|---|---|---|---|---|---|---|---|
| ATOM | 408 | OD1 | ASN A | 450 | 25.269 | 30.731 | 68.840 | 1.00 | 18.52 | O |

| | | | | | | | | | | | |
|---|---|---|---|---|---|---|---|---|---|---|---|
| ANISOU | 408 | OD1 | ASN A | 450 | 2358 | 2427 | 2254 | −27 | 104 | 172 | O |

| | | | | | | | | | | |
|---|---|---|---|---|---|---|---|---|---|---|
| ATOM | 409 | ND2 | ASN A | 450 | 25.140 | 32.369 | 67.258 | 1.00 | 18.94 | N |

| | | | | | | | | | | | |
|---|---|---|---|---|---|---|---|---|---|---|---|
| ANISOU | 409 | ND2 | ASN A | 450 | 2542 | 2653 | 2001 | −85 | 151 | 203 | N |

| | | | | | | | | | | |
|---|---|---|---|---|---|---|---|---|---|---|
| ATOM | 410 | N | ASP A | 451 | 22.010 | 30.650 | 69.740 | 1.00 | 16.71 | N |

| | | | | | | | | | | | |
|---|---|---|---|---|---|---|---|---|---|---|---|
| ANISOU | 410 | N | ASP A | 451 | 2313 | 2006 | 2029 | −86 | 53 | −7 | N |

| | | | | | | | | | | |
|---|---|---|---|---|---|---|---|---|---|---|
| ATOM | 411 | CA | ASP A | 451 | 21.294 | 29.362 | 69.628 | 1.00 | 18.13 | C |

| | | | | | | | | | | | |
|---|---|---|---|---|---|---|---|---|---|---|---|
| ANISOU | 411 | CA | ASP A | 451 | 2292 | 2124 | 2473 | −48 | 66 | −59 | C |

| | | | | | | | | | | |
|---|---|---|---|---|---|---|---|---|---|---|
| ATOM | 412 | C | ASP A | 451 | 22.162 | 28.253 | 69.066 | 1.00 | 18.12 | C |

| | | | | | | | | | | | |
|---|---|---|---|---|---|---|---|---|---|---|---|
| ANISOU | 412 | C | ASP A | 451 | 2461 | 2210 | 2213 | −31 | −7 | −51 | C |

| | | | | | | | | | | |
|---|---|---|---|---|---|---|---|---|---|---|
| ATOM | 413 | O | ASP A | 451 | 21.901 | 27.089 | 69.356 | 1.00 | 17.38 | O |

| | | | | | | | | | | | |
|---|---|---|---|---|---|---|---|---|---|---|---|
| ANISOU | 413 | O | ASP A | 451 | 2498 | 2203 | 1904 | −9 | 77 | 111 | O |

| | | | | | | | | | | |
|---|---|---|---|---|---|---|---|---|---|---|
| ATOM | 414 | CB | ASP A | 451 | 20.045 | 29.564 | 68.761 | 1.00 | 20.04 | C |

| | | | | | | | | | | | |
|---|---|---|---|---|---|---|---|---|---|---|---|
| ANISOU | 414 | CB | ASP A | 451 | 2600 | 2647 | 2367 | −27 | −12 | 58 | C |

| | | | | | | | | | | |
|---|---|---|---|---|---|---|---|---|---|---|
| ATOM | 415 | CG | ASP A | 451 | 20.408 | 30.031 | 67.341 | 1.00 | 21.13 | C |

| | | | | | | | | | | | |
|---|---|---|---|---|---|---|---|---|---|---|---|
| ANISOU | 415 | CG | ASP A | 451 | 2799 | 2721 | 2507 | −15 | 54 | 115 | C |

| | | | | | | | | | | |
|---|---|---|---|---|---|---|---|---|---|---|
| ATOM | 416 | OD1 | ASP A | 451 | 20.501 | 31.259 | 67.145 | 1.00 | 21.53 | O |

| | | | | | | | | | | | |
|---|---|---|---|---|---|---|---|---|---|---|---|
| ANISOU | 416 | OD1 | ASP A | 451 | 2841 | 2714 | 2626 | 6 | −9 | 133 | O |

| | | | | | | | | | | |
|---|---|---|---|---|---|---|---|---|---|---|
| ATOM | 417 | OD2 | ASP A | 451 | 20.625 | 29.148 | 66.500 | 1.00 | 21.94 | O |

| | | | | | | | | | | | |
|---|---|---|---|---|---|---|---|---|---|---|---|
| ANISOU | 417 | OD2 | ASP A | 451 | 3021 | 2743 | 2575 | −29 | −103 | 39 | O |

| | | | | | | | | | | |
|---|---|---|---|---|---|---|---|---|---|---|
| ATOM | 418 | N | THR A | 452 | 23.146 | 28.570 | 68.237 | 1.00 | 17.61 | N |

| | | | | | | | | | | | |
|---|---|---|---|---|---|---|---|---|---|---|---|
| ANISOU | 418 | N | THR A | 452 | 2184 | 2145 | 2364 | 9 | 19 | −3 | N |

| | | | | | | | | | | |
|---|---|---|---|---|---|---|---|---|---|---|
| ATOM | 419 | CA | THR A | 452 | 24.010 | 27.552 | 67.636 | 1.00 | 17.98 | C |

| | | | | | | | | | | | |
|---|---|---|---|---|---|---|---|---|---|---|---|
| ANISOU | 419 | CA | THR A | 452 | 2468 | 2252 | 2113 | −41 | 144 | −106 | C |

| | | | | | | | | | | |
|---|---|---|---|---|---|---|---|---|---|---|
| ATOM | 420 | C | THR A | 452 | 24.854 | 26.844 | 68.653 | 1.00 | 17.29 | C |

| | | | | | | | | | | | |
|---|---|---|---|---|---|---|---|---|---|---|---|
| ANISOU | 420 | C | THR A | 452 | 2412 | 2199 | 1958 | −97 | 242 | −76 | C |

| | | | | | | | | | | |
|---|---|---|---|---|---|---|---|---|---|---|
| ATOM | 421 | O | THR A | 452 | 24.979 | 25.617 | 68.675 | 1.00 | 17.49 | O |

| | | | | | | | | | | | |
|---|---|---|---|---|---|---|---|---|---|---|---|
| ANISOU | 421 | O | THR A | 452 | 2619 | 2167 | 1860 | 20 | 82 | −265 | O |

| | | | | | | | | | | |
|---|---|---|---|---|---|---|---|---|---|---|
| ATOM | 422 | CB | THR A | 452 | 24.927 | 28.219 | 66.582 | 1.00 | 18.78 | C |

| | | | | | | | | | | | |
|---|---|---|---|---|---|---|---|---|---|---|---|
| ANISOU | 422 | CB | THR A | 452 | 2403 | 2352 | 2379 | −20 | 156 | 109 | C |

| | | | | | | | | | | |
|---|---|---|---|---|---|---|---|---|---|---|
| ATOM | 423 | OG1 | THR A | 452 | 24.088 | 29.014 | 65.715 | 1.00 | 21.03 | O |

| | | | | | | | | | | | |
|---|---|---|---|---|---|---|---|---|---|---|---|
| ANISOU | 423 | OG1 | THR A | 452 | 2868 | 2760 | 2364 | 170 | 37 | 107 | O |

| | | | | | | | | | | |
|---|---|---|---|---|---|---|---|---|---|---|
| ATOM | 424 | CG2 | THR A | 452 | 25.681 | 27.149 | 65.829 | 1.00 | 17.83 | C |

| | | | | | | | | | | | |
|---|---|---|---|---|---|---|---|---|---|---|---|
| ANISOU | 424 | CG2 | THR A | 452 | 2391 | 2303 | 2081 | −118 | −51 | 20 | C |

| | | | | | | | | | | |
|---|---|---|---|---|---|---|---|---|---|---|
| ATOM | 425 | N | ILE A | 453 | 25.409 | 27.621 | 69.597 | 1.00 | 17.00 | N |

| | | | | | | | | | | | |
|---|---|---|---|---|---|---|---|---|---|---|---|
| ANISOU | 425 | N | ILE A | 453 | 2383 | 2146 | 1932 | −12 | 39 | −57 | N |

| | | | | | | | | | | |
|---|---|---|---|---|---|---|---|---|---|---|
| ATOM | 426 | CA | ILE A | 453 | 26.202 | 27.037 | 70.678 | 1.00 | 16.62 | C |

| | | | | | | | | | | | |
|---|---|---|---|---|---|---|---|---|---|---|---|
| ANISOU | 426 | CA | ILE A | 453 | 2135 | 2182 | 1998 | −137 | 3 | 26 | C |

| | | | | | | | | | | |
|---|---|---|---|---|---|---|---|---|---|---|
| ATOM | 427 | C | ILE A | 453 | 25.319 | 26.242 | 71.631 | 1.00 | 15.53 | C |

| | | | | | | | | | | | |
|---|---|---|---|---|---|---|---|---|---|---|---|
| ANISOU | 427 | C | ILE A | 453 | 2092 | 2103 | 1705 | 37 | 10 | −45 | C |

| | | | | | | | | | | |
|---|---|---|---|---|---|---|---|---|---|---|
| ATOM | 428 | O | ILE A | 453 | 25.675 | 25.148 | 72.069 | 1.00 | 16.87 | O |

| | | | | | | | | | | | |
|---|---|---|---|---|---|---|---|---|---|---|---|
| ANISOU | 428 | O | ILE A | 453 | 2307 | 2095 | 2006 | −66 | 92 | 4 | O |

| | | | | | | | | | | |
|---|---|---|---|---|---|---|---|---|---|---|
| ATOM | 429 | CB | ILE A | 453 | 27.012 | 28.127 | 71.415 | 1.00 | 15.67 | C |

| | | | | | | | | | | | |
|---|---|---|---|---|---|---|---|---|---|---|---|
| ANISOU | 429 | CB | ILE A | 453 | 2109 | 1918 | 1927 | −84 | 74 | 52 | C |

| | | | | | | | | | | |
|---|---|---|---|---|---|---|---|---|---|---|
| ATOM | 430 | CG1 | ILE A | 453 | 27.941 | 28.842 | 70.404 | 1.00 | 16.39 | C |

| | | | | | | | | | | | |
|---|---|---|---|---|---|---|---|---|---|---|---|
| ANISOU | 430 | CG1 | ILE A | 453 | 2156 | 2103 | 1970 | −73 | 107 | −45 | C |

| | | | | | | | | | | |
|---|---|---|---|---|---|---|---|---|---|---|
| ATOM | 431 | CG2 | ILE A | 453 | 27.780 | 27.454 | 72.545 | 1.00 | 16.95 | C |

| | | | | | | | | | | | |
|---|---|---|---|---|---|---|---|---|---|---|---|
| ANISOU | 431 | CG2 | ILE A | 453 | 2183 | 2332 | 1923 | 29 | −87 | −101 | C |

| | | | | | | | | | | |
|---|---|---|---|---|---|---|---|---|---|---|
| ATOM | 432 | CD1 | ILE A | 453 | 28.868 | 27.950 | 69.578 | 1.00 | 17.17 | C |

| | | | | | | | | | | | |
|---|---|---|---|---|---|---|---|---|---|---|---|
| ANISOU | 432 | CD1 | ILE A | 453 | 2361 | 2131 | 2034 | 31 | 129 | −48 | C |

| | | | | | | | | | | |
|---|---|---|---|---|---|---|---|---|---|---|
| ATOM | 433 | N | ILE A | 454 | 24.133 | 26.778 | 71.985 | 1.00 | 16.23 | N |

| | | | | | | | | | | | |
|---|---|---|---|---|---|---|---|---|---|---|---|
| ANISOU | 433 | N | ILE A | 454 | 2058 | 2309 | 1801 | 31 | 123 | −109 | N |

| | | | | | | | | | | |
|---|---|---|---|---|---|---|---|---|---|---|
| ATOM | 434 | CA | ILE A | 454 | 23.193 | 26.021 | 72.822 | 1.00 | 16.62 | C |

| | | | | | | | | | | | |
|---|---|---|---|---|---|---|---|---|---|---|---|
| ANISOU | 434 | CA | ILE A | 454 | 2283 | 1992 | 2041 | −18 | 54 | −31 | C |

| | | | | | | | | | | |
|---|---|---|---|---|---|---|---|---|---|---|
| ATOM | 435 | C | ILE A | 454 | 22.830 | 24.702 | 72.114 | 1.00 | 17.03 | C |

| | | | | | | | | | | | |
|---|---|---|---|---|---|---|---|---|---|---|---|
| ANISOU | 435 | C | ILE A | 454 | 2193 | 2045 | 2234 | −92 | 81 | −41 | C |

| | | | | | | | | | | |
|---|---|---|---|---|---|---|---|---|---|---|
| ATOM | 436 | O | ILE A | 454 | 22.891 | 23.656 | 72.787 | 1.00 | 16.67 | O |

| | | | | | | | | | | | |
|---|---|---|---|---|---|---|---|---|---|---|---|
| ANISOU | 436 | O | ILE A | 454 | 2264 | 1963 | 2107 | −34 | 174 | −102 | O |

| | | | | | | | | | | |
|---|---|---|---|---|---|---|---|---|---|---|
| ATOM | 437 | CB | ILE A | 454 | 21.894 | 26.853 | 73.011 | 1.00 | 17.04 | C |

| | | | | | | | | | | | |
|---|---|---|---|---|---|---|---|---|---|---|---|
| ANISOU | 437 | CB | ILE A | 454 | 2153 | 2215 | 2105 | −64 | 127 | 7 | C |

| | | | | | | | | | | |
|---|---|---|---|---|---|---|---|---|---|---|
| ATOM | 438 | CG1 | ILE A | 454 | 22.190 | 28.126 | 73.809 | 1.00 | 18.96 | C |

| | | | | | | | | | | | |
|---|---|---|---|---|---|---|---|---|---|---|---|
| ANISOU | 438 | CG1 | ILE A | 454 | 2510 | 2180 | 2512 | −73 | 22 | −2 | C |

| | | | | | | | | | | |
|---|---|---|---|---|---|---|---|---|---|---|
| ATOM | 439 | CG2 | ILE A | 454 | 20.810 | 25.981 | 73.661 | 1.00 | 17.94 | C |

| | | | | | | | | | | | |
|---|---|---|---|---|---|---|---|---|---|---|---|
| ANISOU | 439 | CG2 | ILE A | 454 | 2037 | 2285 | 2496 | 21 | 226 | 46 | C |

| | | | | | | | | | | |
|---|---|---|---|---|---|---|---|---|---|---|
| ATOM | 440 | CD1 | ILE A | 454 | 22.608 | 27.891 | 75.239 | 1.00 | 20.59 | C |

| | | | | | | | | | | | |
|---|---|---|---|---|---|---|---|---|---|---|---|
| ANISOU | 440 | CD1 | ILE A | 454 | 2780 | 2440 | 2603 | −43 | −5 | 112 | C |

| | | | | | | | | | | |
|---|---|---|---|---|---|---|---|---|---|---|
| ATOM | 441 | N | GLU A | 455 | 22.571 | 24.722 | 70.813 | 1.00 | 17.22 | N |

| | | | | | | | | | | | |
|---|---|---|---|---|---|---|---|---|---|---|---|
| ANISOU | 441 | N | GLU A | 455 | 2228 | 2031 | 2283 | −15 | 31 | 33 | N |

| | | | | | | | | | | |
|---|---|---|---|---|---|---|---|---|---|---|
| ATOM | 442 | CA | GLU A | 455 | 22.177 | 23.478 | 70.134 | 1.00 | 18.12 | C |

| | | | | | | | | | | | |
|---|---|---|---|---|---|---|---|---|---|---|---|
| ANISOU | 442 | CA | GLU A | 455 | 2238 | 2312 | 2337 | 3 | 25 | −68 | C |

| | | | | | | | | | | |
|---|---|---|---|---|---|---|---|---|---|---|
| ATOM | 443 | C | GLU A | 455 | 23.338 | 22.478 | 70.115 | 1.00 | 19.31 | C |

| | | | | | | | | | | | |
|---|---|---|---|---|---|---|---|---|---|---|---|
| ANISOU | 443 | C | GLU A | 455 | 2423 | 2399 | 2513 | −49 | −1 | 57 | C |

| | | | | | | | | | | |
|---|---|---|---|---|---|---|---|---|---|---|
| ATOM | 444 | O | GLU A | 455 | 23.156 | 21.284 | 70.345 | 1.00 | 19.67 | O |

| | | | | | | | | | | | |
|---|---|---|---|---|---|---|---|---|---|---|---|
| ANISOU | 444 | O | GLU A | 455 | 2602 | 2216 | 2654 | 72 | −57 | −124 | O |

| | | | | | | | | | | |
|---|---|---|---|---|---|---|---|---|---|---|
| ATOM | 445 | CB | GLU A | 455 | 21.698 | 23.761 | 68.713 | 1.00 | 19.41 | C |

| | | | | | | | | | | | |
|---|---|---|---|---|---|---|---|---|---|---|---|
| ANISOU | 445 | CB | GLU A | 455 | 2682 | 2388 | 2306 | −73 | −32 | −33 | C |

| | | | | | | | | | | |
|---|---|---|---|---|---|---|---|---|---|---|
| ATOM | 446 | CG | GLU A | 455 | 21.251 | 22.457 | 68.040 | 1.00 | 21.19 | C |

| | | | | | | | | | | | |
|---|---|---|---|---|---|---|---|---|---|---|---|
| ANISOU | 446 | CG | GLU A | 455 | 2963 | 2470 | 2619 | 3 | −10 | −185 | C |

| | | | | | | | | | | |
|---|---|---|---|---|---|---|---|---|---|---|
| ATOM | 447 | CD | GLU A | 455 | 20.417 | 22.586 | 66.803 | 1.00 | 22.79 | C |

| | | | | | | | | | | | |
|---|---|---|---|---|---|---|---|---|---|---|---|
| ANISOU | 447 | CD | GLU A | 455 | 2987 | 2925 | 2748 | −18 | −34 | −157 | C |

| | | | | | | | | | | |
|---|---|---|---|---|---|---|---|---|---|---|
| ATOM | 448 | OE1 | GLU A | 455 | 20.242 | 23.696 | 66.274 | 1.00 | 23.95 | O |

| | | | | | | | | | | | |
|---|---|---|---|---|---|---|---|---|---|---|---|
| ANISOU | 448 | OE1 | GLU A | 455 | 3301 | 2994 | 2804 | −97 | −66 | −171 | O |

| | | | | | | | | | | |
|---|---|---|---|---|---|---|---|---|---|---|
| ATOM | 449 | OE2 | GLU A | 455 | 19.885 | 21.537 | 66.393 | 1.00 | 23.35 | O |

| | | | | | | | | | | | |
|---|---|---|---|---|---|---|---|---|---|---|---|
| ANISOU | 449 | OE2 | GLU A | 455 | 3067 | 3011 | 2796 | −30 | −122 | −346 | O |

| | | | | | | | | | | |
|---|---|---|---|---|---|---|---|---|---|---|
| ATOM | 450 | N | PHE A | 456 | 24.534 | 23.010 | 69.840 | 1.00 | 18.48 | N |

| | | | | | | | | | | | |
|---|---|---|---|---|---|---|---|---|---|---|---|
| ANISOU | 450 | N | PHE A | 456 | 2280 | 2217 | 2525 | 55 | 160 | −114 | N |

| | | | | | | | | | | |
|---|---|---|---|---|---|---|---|---|---|---|
| ATOM | 451 | CA | PHE A | 456 | 25.708 | 22.117 | 69.855 | 1.00 | 16.99 | C |

| | | | | | | | | | | | |
|---|---|---|---|---|---|---|---|---|---|---|---|
| ANISOU | 451 | CA | PHE A | 456 | 1926 | 2110 | 2421 | −34 | −87 | −69 | C |

| | | | | | | | | | | |
|---|---|---|---|---|---|---|---|---|---|---|
| ATOM | 452 | C | PHE A | 456 | 25.801 | 21.417 | 71.207 | 1.00 | 17.77 | C |

| | | | | | | | | | | | |
|---|---|---|---|---|---|---|---|---|---|---|---|
| ANISOU | 452 | C | PHE A | 456 | 2112 | 2216 | 2426 | −75 | −39 | −21 | C |

| | | | | | | | | | | |
|---|---|---|---|---|---|---|---|---|---|---|
| ATOM | 453 | O | PHE A | 456 | 26.034 | 20.189 | 71.267 | 1.00 | 17.72 | O |

| | | | | | | | | | | | |
|---|---|---|---|---|---|---|---|---|---|---|---|
| ANISOU | 453 | O | PHE A | 456 | 2169 | 2172 | 2390 | −14 | 131 | −59 | O |

| | | | | | | | | | | |
|---|---|---|---|---|---|---|---|---|---|---|
| ATOM | 454 | CB | PHE A | 456 | 27.038 | 22.894 | 69.617 | 1.00 | 17.46 | C |

| | | | | | | | | | | | |
|---|---|---|---|---|---|---|---|---|---|---|---|
| ANISOU | 454 | CB | PHE A | 456 | 2094 | 1977 | 2561 | −25 | −30 | −76 | C |

| | | | | | | | | | | |
|---|---|---|---|---|---|---|---|---|---|---|
| ATOM | 455 | CG | PHE A | 456 | 28.233 | 22.020 | 69.912 | 1.00 | 18.81 | C |

| | | | | | | | | | | | |
|---|---|---|---|---|---|---|---|---|---|---|---|
| ANISOU | 455 | CG | PHE A | 456 | 2537 | 2248 | 2363 | 85 | 5 | −103 | C |

| | | | | | | | | | | |
|---|---|---|---|---|---|---|---|---|---|---|
| ATOM | 456 | CD1 | PHE A | 456 | 28.627 | 21.018 | 69.041 | 1.00 | 19.99 | C |

| | | | | | | | | | | | |
|---|---|---|---|---|---|---|---|---|---|---|---|
| ANISOU | 456 | CD1 | PHE A | 456 | 2549 | 2399 | 2647 | 164 | −32 | −139 | C |

| | | | | | | | | | | |
|---|---|---|---|---|---|---|---|---|---|---|
| ATOM | 457 | CD2 | PHE A | 456 | 28.912 | 22.152 | 71.109 | 1.00 | 17.34 | C |

| | | | | | | | | | | | |
|---|---|---|---|---|---|---|---|---|---|---|---|
| ANISOU | 457 | CD2 | PHE A | 456 | 1873 | 2165 | 2550 | 116 | −70 | −27 | C |

| | | | | | | | | | | |
|---|---|---|---|---|---|---|---|---|---|---|
| ATOM | 458 | CE1 | PHE A | 456 | 29.701 | 20.207 | 69.339 | 1.00 | 21.78 | C |

| | | | | | | | | | | | |
|---|---|---|---|---|---|---|---|---|---|---|---|
| ANISOU | 458 | CE1 | PHE A | 456 | 2865 | 2632 | 2778 | 152 | −135 | −83 | C |

| | | | | | | | | | | |
|---|---|---|---|---|---|---|---|---|---|---|
| ATOM | 459 | CE2 | PHE A | 456 | 30.007 | 21.367 | 71.441 | 1.00 | 18.50 | C |

| | | | | | | | | | | | |
|---|---|---|---|---|---|---|---|---|---|---|---|
| ANISOU | 459 | CE2 | PHE A | 456 | 2489 | 2110 | 2429 | 92 | 47 | 5 | C |

| | | | | | | | | | | |
|---|---|---|---|---|---|---|---|---|---|---|
| ATOM | 460 | CZ | PHE A | 456 | 30.376 | 20.381 | 70.544 | 1.00 | 20.03 | C |

| | | | | | | | | | | | |
|---|---|---|---|---|---|---|---|---|---|---|---|
| ANISOU | 460 | CZ | PHE A | 456 | 2484 | 2431 | 2696 | 202 | 49 | −140 | C |

| | | | | | | | | | | |
|---|---|---|---|---|---|---|---|---|---|---|
| ATOM | 461 | N | PHE A | 457 | 25.649 | 22.147 | 72.309 | 1.00 | 17.68 | N |

| | | | | | | | | | | | |
|---|---|---|---|---|---|---|---|---|---|---|---|
| ANISOU | 461 | N | PHE A | 457 | 2221 | 2042 | 2455 | 22 | −99 | −16 | N |

| | | | | | | | | | | |
|---|---|---|---|---|---|---|---|---|---|---|
| ATOM | 462 | CA | PHE A | 457 | 25.779 | 21.509 | 73.619 | 1.00 | 17.30 | C |

| | | | | | | | | | | | |
|---|---|---|---|---|---|---|---|---|---|---|---|
| ANISOU | 462 | CA | PHE A | 457 | 2163 | 2093 | 2327 | −67 | −8 | −1 | C |

| | | | | | | | | | | |
|---|---|---|---|---|---|---|---|---|---|---|
| ATOM | 463 | C | PHE A | 457 | 24.667 | 20.504 | 73.902 | 1.00 | 17.52 | C |

| | | | | | | | | | | | |
|---|---|---|---|---|---|---|---|---|---|---|---|
| ANISOU | 463 | C | PHE A | 457 | 2224 | 2034 | 2401 | −6 | −66 | −152 | C |

| | | | | | | | | | | |
|---|---|---|---|---|---|---|---|---|---|---|
| ATOM | 464 | O | PHE A | 457 | 24.959 | 19.421 | 74.436 | 1.00 | 17.26 | O |

| | | | | | | | | | | | |
|---|---|---|---|---|---|---|---|---|---|---|---|
| ANISOU | 464 | O | PHE A | 457 | 2118 | 2119 | 2319 | 45 | −4 | −80 | O |

| | | | | | | | | | | |
|---|---|---|---|---|---|---|---|---|---|---|
| ATOM | 465 | CB | PHE A | 457 | 25.939 | 22.527 | 74.752 | 1.00 | 19.77 | C |

| | | | | | | | | | | | |
|---|---|---|---|---|---|---|---|---|---|---|---|
| ANISOU | 465 | CB | PHE A | 457 | 2526 | 2129 | 2476 | 119 | −52 | −120 | C |

| | | | | | | | | | | |
|---|---|---|---|---|---|---|---|---|---|---|
| ATOM | 466 | CD | PHE A | 457 | 26.522 | 21.867 | 75.981 | 1.00 | 18.03 | C |

| | | | | | | | | | | | |
|---|---|---|---|---|---|---|---|---|---|---|---|
| ANISOU | 466 | CD | PHE A | 457 | 2197 | 2162 | 2490 | 112 | 25 | −130 | C |

| | | | | | | | | | | |
|---|---|---|---|---|---|---|---|---|---|---|
| ATOM | 467 | CD1 | PHE A | 457 | 27.910 | 21.562 | 75.985 | 1.00 | 18.56 | C |

| | | | | | | | | | | | |
|---|---|---|---|---|---|---|---|---|---|---|---|
| ANISOU | 467 | CD1 | PHE A | 457 | 2185 | 2403 | 2465 | −149 | 33 | 29 | C |

| | | | | | | | | | | |
|---|---|---|---|---|---|---|---|---|---|---|
| ATOM | 468 | CD2 | PHE A | 457 | 25.743 | 21.538 | 77.054 | 1.00 | 18.50 | C |

| | | | | | | | | | | | |
|---|---|---|---|---|---|---|---|---|---|---|---|
| ANISOU | 468 | CD2 | PHE A | 457 | 2685 | 1997 | 2349 | 14 | −56 | −28 | C |

| | | | | | | | | | | |
|---|---|---|---|---|---|---|---|---|---|---|
| ATOM | 469 | CE1 | PHE A | 457 | 28.437 | 20.917 | 77.096 | 1.00 | 17.93 | C |

| | | | | | | | | | | | |
|---|---|---|---|---|---|---|---|---|---|---|---|
| ANISOU | 469 | CE1 | PHE A | 457 | 2126 | 2104 | 2582 | −131 | −59 | −38 | C |

| | | | | | | | | | | |
|---|---|---|---|---|---|---|---|---|---|---|
| ATOM | 470 | CE2 | PHE A | 457 | 26.273 | 20.898 | 78.174 | 1.00 | 18.60 | C |

| | | | | | | | | | | | |
|---|---|---|---|---|---|---|---|---|---|---|---|
| ANISOU | 470 | CE2 | PHE A | 457 | 2472 | 2173 | 2420 | 125 | −115 | −157 | C |

| | | | | | | | | | | |
|---|---|---|---|---|---|---|---|---|---|---|
| ATOM | 471 | CZ | PHE A | 457 | 27.608 | 20.582 | 78.178 | 1.00 | 18.40 | C |

| | | | | | | | | | | | |
|---|---|---|---|---|---|---|---|---|---|---|---|
| ANISOU | 471 | CZ | PHE A | 457 | 2452 | 2132 | 2409 | −240 | −78 | −176 | C |

| | | | | | | | | | | |
|---|---|---|---|---|---|---|---|---|---|---|
| ATOM | 472 | N | MET A | 458 | 23.474 | 20.772 | 73.430 | 1.00 | 16.73 | N |

| | | | | | | | | | | | |
|---|---|---|---|---|---|---|---|---|---|---|---|
| ANISOU | 472 | N | MET A | 458 | 2215 | 1843 | 2297 | 87 | 31 | −239 | N |

| | | | | | | | | | | |
|---|---|---|---|---|---|---|---|---|---|---|
| ATOM | 473 | CA | MET A | 458 | 22.376 | 19.810 | 73.550 | 1.00 | 16.99 | C |

| | | | | | | | | | | | |
|---|---|---|---|---|---|---|---|---|---|---|---|
| ANISOU | 473 | CA | MET A | 458 | 2174 | 1730 | 2552 | 85 | 40 | −213 | C |

| | | | | | | | | | | |
|---|---|---|---|---|---|---|---|---|---|---|
| ATOM | 474 | C | MET A | 458 | 22.774 | 18.520 | 72.788 | 1.00 | 17.29 | C |

| | | | | | | | | | | | |
|---|---|---|---|---|---|---|---|---|---|---|---|
| ANISOU | 474 | C | MET A | 458 | 2150 | 1938 | 2482 | 100 | 96 | −291 | C |

| | | | | | | | | | | |
|---|---|---|---|---|---|---|---|---|---|---|
| ATOM | 475 | O | MET A | 458 | 22.569 | 17.419 | 73.319 | 1.00 | 19.89 | O |

| | | | | | | | | | | | |
|---|---|---|---|---|---|---|---|---|---|---|---|
| ANISOU | 475 | O | MET A | 458 | 2409 | 2255 | 2893 | 24 | −70 | −115 | O |

| | | | | | | | | | | |
|---|---|---|---|---|---|---|---|---|---|---|
| ATOM | 476 | CB | MET A | 458 | 21.054 | 20.373 | 73.014 | 1.00 | 16.73 | C |

| | | | | | | | | | | | |
|---|---|---|---|---|---|---|---|---|---|---|---|
| ANISOU | 476 | CB | MET A | 458 | 2411 | 1892 | 2055 | 156 | −29 | −197 | C |

| | | | | | | | | | | |
|---|---|---|---|---|---|---|---|---|---|---|
| ATOM | 477 | CG | MET A | 458 | 20.554 | 21.631 | 73.744 | 1.00 | 17.55 | C |

| | | | | | | | | | | | |
|---|---|---|---|---|---|---|---|---|---|---|---|
| ANISOU | 477 | CG | MET A | 458 | 2396 | 1712 | 2561 | 76 | 142 | −111 | C |

| | | | | | | | | | | |
|---|---|---|---|---|---|---|---|---|---|---|
| ATOM | 478 | SD | MET A | 458 | 20.296 | 21.390 | 75.504 | 1.00 | 17.18 | S |

| | | | | | | | | | | | |
|---|---|---|---|---|---|---|---|---|---|---|---|
| ANISOU | 478 | SD | MET A | 458 | 2306 | 1587 | 2633 | −60 | 230 | −71 | S |

| | | | | | | | | | | |
|---|---|---|---|---|---|---|---|---|---|---|
| ATOM | 479 | CE | MET A | 458 | 21.743 | 22.189 | 76.188 | 1.00 | 19.31 | C |

| | | | | | | | | | | | |
|---|---|---|---|---|---|---|---|---|---|---|---|
| ANISOU | 479 | CE | MET A | 458 | 2814 | 2040 | 2482 | −237 | 38 | −208 | C |

| | | | | | | | | | | |
|---|---|---|---|---|---|---|---|---|---|---|
| ATOM | 480 | N | LYS A | 459 | 23.329 | 18.660 | 71.604 | 1.00 | 17.26 | N |

| | | | | | | | | | | | |
|---|---|---|---|---|---|---|---|---|---|---|---|
| ANISOU | 480 | N | LYS A | 459 | 2356 | 1811 | 2392 | 186 | −64 | −198 | N |

| | | | | | | | | | | |
|---|---|---|---|---|---|---|---|---|---|---|
| ATOM | 481 | CA | LYS A | 459 | 23.750 | 17.460 | 70.828 | 1.00 | 19.22 | C |

| | | | | | | | | | | | |
|---|---|---|---|---|---|---|---|---|---|---|---|
| ANISOU | 481 | CA | LYS A | 459 | 2632 | 2005 | 2665 | 152 | 72 | −251 | C |

| | | | | | | | | | | |
|---|---|---|---|---|---|---|---|---|---|---|
| ATOM | 482 | C | LYS A | 459 | 24.822 | 16.681 | 71.551 | 1.00 | 20.55 | C |

| | | | | | | | | | | | |
|---|---|---|---|---|---|---|---|---|---|---|---|
| ANISOU | 482 | C | LYS A | 459 | 2568 | 2368 | 2871 | 24 | −100 | −94 | C |

| | | | | | | | | | | |
|---|---|---|---|---|---|---|---|---|---|---|
| ATOM | 483 | O | LYS A | 459 | 24.834 | 15.431 | 71.585 | 1.00 | 22.61 | O |

| | | | | | | | | | | | |
|---|---|---|---|---|---|---|---|---|---|---|---|
| ANISOU | 483 | O | LYS A | 459 | 2828 | 2342 | 3423 | 117 | −89 | −148 | O |

| | | | | | | | | | | |
|---|---|---|---|---|---|---|---|---|---|---|
| ATOM | 484 | CB | LYS A | 459 | 24.269 | 17.893 | 69.449 | 1.00 | 20.32 | C |

| | | | | | | | | | | | |
|---|---|---|---|---|---|---|---|---|---|---|---|
| ANISOU | 484 | CB | LYS A | 459 | 2664 | 2381 | 2676 | 325 | 187 | −201 | C |

| | | | | | | | | | | |
|---|---|---|---|---|---|---|---|---|---|---|
| ATOM | 485 | CG | LYS A | 459 | 23.133 | 18.323 | 68.503 | 1.00 | 24.79 | C |

| | | | | | | | | | | | |
|---|---|---|---|---|---|---|---|---|---|---|---|
| ANISOU | 485 | CG | LYS A | 459 | 2792 | 3252 | 3376 | 249 | −70 | −17 | C |

| | | | | | | | | | | |
|---|---|---|---|---|---|---|---|---|---|---|
| ATOM | 486 | CD | LYS A | 459 | 22.501 | 17.176 | 67.749 | 1.00 | 30.15 | C |

| | | | | | | | | | | | |
|---|---|---|---|---|---|---|---|---|---|---|---|
| ANISOU | 486 | CD | LYS A | 459 | 3840 | 3746 | 3871 | 10 | −90 | −200 | C |

| | | | | | | | | | | |
|---|---|---|---|---|---|---|---|---|---|---|
| ATOM | 487 | CE | LYS A | 459 | 21.777 | 17.804 | 66.526 | 1.00 | 32.80 | C |

| | | | | | | | | | | | |
|---|---|---|---|---|---|---|---|---|---|---|---|
| ANISOU | 487 | CE | LYS A | 459 | 3972 | 4352 | 4140 | 233 | −87 | 13 | C |

| | | | | | | | | | | |
|---|---|---|---|---|---|---|---|---|---|---|
| ATOM | 488 | NZ | LYS A | 459 | 21.337 | 16.664 | 65.686 | 1.00 | 35.13 | N |

| | | | | | | | | | | | |
|---|---|---|---|---|---|---|---|---|---|---|---|
| ANISOU | 488 | NZ | LYS A | 459 | 4317 | 4691 | 4339 | −1 | −12 | −136 | N |

| | | | | | | | | | | |
|---|---|---|---|---|---|---|---|---|---|---|
| ATOM | 489 | N | TYR A | 460 | 25.790 | 17.411 | 72.123 | 1.00 | 19.93 | N |

| | | | | | | | | | | | |
|---|---|---|---|---|---|---|---|---|---|---|---|
| ANISOU | 489 | N | TYR A | 460 | 2247 | 2284 | 3042 | 121 | 13 | −187 | N |

| | | | | | | | | | | |
|---|---|---|---|---|---|---|---|---|---|---|
| ATOM | 490 | CA | TYR A | 460 | 26.836 | 16.734 | 72.895 | 1.00 | 19.98 | C |

| | | | | | | | | | | | |
|---|---|---|---|---|---|---|---|---|---|---|---|
| ANISOU | 490 | CA | TYR A | 460 | 2643 | 2222 | 2726 | 40 | −57 | −28 | C |

| | | | | | | | | | | |
|---|---|---|---|---|---|---|---|---|---|---|
| ATOM | 491 | C | TYR A | 460 | 26.267 | 15.906 | 74.054 | 1.00 | 20.30 | O |

| | | | | | | | | | | | |
|---|---|---|---|---|---|---|---|---|---|---|---|
| ANISOU | 491 | C | TYR A | 460 | 2451 | 2387 | 2873 | 96 | 182 | −33 | C |

| | | | | | | | | | | |
|---|---|---|---|---|---|---|---|---|---|---|
| ATOM | 492 | O | TYR A | 460 | 26.725 | 14.793 | 74.371 | 1.00 | 20.01 | O |

| | | | | | | | | | | | |
|---|---|---|---|---|---|---|---|---|---|---|---|
| ANISOU | 492 | O | TYR A | 460 | 2590 | 2152 | 2860 | 70 | −39 | −153 | O |

| | | | | | | | | | | |
|---|---|---|---|---|---|---|---|---|---|---|
| ATOM | 493 | CB | TYR A | 460 | 27.835 | 17.786 | 73.370 | 1.00 | 18.90 | C |

| | | | | | | | | | | | |
|---|---|---|---|---|---|---|---|---|---|---|---|
| ANISOU | 493 | CB | TYR A | 460 | 2106 | 2345 | 2730 | 97 | 156 | −181 | C |

| | | | | | | | | | | |
|---|---|---|---|---|---|---|---|---|---|---|
| ATOM | 494 | CG | TYR A | 460 | 28.779 | 17.336 | 74.450 | 1.00 | 20.69 | C |

| | | | | | | | | | | | |
|---|---|---|---|---|---|---|---|---|---|---|---|
| ANISOU | 494 | CG | TYR A | 460 | 2703 | 2332 | 2826 | −30 | 45 | 2 | C |

| | | | | | | | | | | |
|---|---|---|---|---|---|---|---|---|---|---|
| ATOM | 495 | CD1 | TYR A | 460 | 29.978 | 16.512 | 74.164 | 1.00 | 20.73 | C |

| | | | | | | | | | | | |
|---|---|---|---|---|---|---|---|---|---|---|---|
| ANISOU | 495 | CD1 | TYR A | 460 | 2584 | 2306 | 2988 | −27 | 70 | 43 | C |

| | | | | | | | | | | |
|---|---|---|---|---|---|---|---|---|---|---|
| ATOM | 496 | CD2 | TYR A | 460 | 29.596 | 17.700 | 75.760 | 1.00 | 20.86 | C |

| | | | | | | | | | | | |
|---|---|---|---|---|---|---|---|---|---|---|---|
| ANISOU | 496 | CD2 | TYR A | 460 | 2665 | 2439 | 2824 | −123 | 99 | 26 | C |

| | | | | | | | | | | |
|---|---|---|---|---|---|---|---|---|---|---|
| ATOM | 497 | CE1 | TYR A | 460 | 30.755 | 16.081 | 75.156 | 1.00 | 21.90 | C |

| | | | | | | | | | | | |
|---|---|---|---|---|---|---|---|---|---|---|---|
| ANISOU | 497 | CE1 | TYR A | 460 | 2809 | 2506 | 3007 | −192 | −57 | 25 | C |

| | | | | | | | | | | |
|---|---|---|---|---|---|---|---|---|---|---|
| ATOM | 498 | CE2 | TYR A | 460 | 29.428 | 17.277 | 76.762 | 1.00 | 22.47 | C |

| | | | | | | | | | | | |
|---|---|---|---|---|---|---|---|---|---|---|---|
| ANISOU | 498 | CE2 | TYR A | 460 | 2771 | 2720 | 3048 | −87 | −61 | 130 | C |

| | | | | | | | | | | |
|---|---|---|---|---|---|---|---|---|---|---|
| ATOM | 499 | CZ | TYR A | 460 | 30.511 | 16.472 | 76.452 | 1.00 | 22.05 | C |

| | | | | | | | | | | | |
|---|---|---|---|---|---|---|---|---|---|---|---|
| ANISOU | 499 | CZ | TYR A | 460 | 2864 | 2592 | 2923 | −153 | 19 | 149 | C |

| | | | | | | | | | | |
|---|---|---|---|---|---|---|---|---|---|---|
| ATOM | 500 | OH | TYR A | 460 | 31.355 | 16.099 | 77.467 | 1.00 | 24.73 | O |

| | | | | | | | | | | | |
|---|---|---|---|---|---|---|---|---|---|---|---|
| ANISOU | 500 | OH | TYR A | 460 | 3230 | 3004 | 3162 | −161 | −142 | 321 | O |

| | | | | | | | | | | |
|---|---|---|---|---|---|---|---|---|---|---|
| ATOM | 501 | N | ILE A | 461 | 25.329 | 16.459 | 74.820 | 1.00 | 19.01 | N |

| | | | | | | | | | | | |
|---|---|---|---|---|---|---|---|---|---|---|---|
| ANISOU | 501 | N | ILE A | 461 | 2697 | 2140 | 2385 | 54 | 99 | −166 | N |

| | | | | | | | | | | |
|---|---|---|---|---|---|---|---|---|---|---|
| ATOM | 502 | CA | ILE A | 461 | 24.677 | 15.743 | 75.910 | 1.00 | 19.87 | C |

| | | | | | | | | | | | |
|---|---|---|---|---|---|---|---|---|---|---|---|
| ANISOU | 502 | CA | ILE A | 461 | 2559 | 2227 | 2761 | 51 | 304 | −134 | C |

| | | | | | | | | | | |
|---|---|---|---|---|---|---|---|---|---|---|
| ATOM | 503 | C | ILE A | 461 | 23.874 | 14.573 | 75.357 | 1.00 | 20.86 | C |

| | | | | | | | | | | | |
|---|---|---|---|---|---|---|---|---|---|---|---|
| ANISOU | 503 | C | ILE A | 461 | 2656 | 2363 | 2909 | −26 | 78 | −52 | C |

| | | | | | | | | | | |
|---|---|---|---|---|---|---|---|---|---|---|
| ATOM | 504 | O | ILE A | 461 | 24.009 | 13.468 | 75.890 | 1.00 | 22.24 | O |

| | | | | | | | | | | | |
|---|---|---|---|---|---|---|---|---|---|---|---|
| ANISOU | 504 | O | ILE A | 461 | 2661 | 2482 | 3309 | −134 | −89 | 96 | O |

| | | | | | | | | | | |
|---|---|---|---|---|---|---|---|---|---|---|
| ATOM | 505 | CB | ILE A | 461 | 23.722 | 16.752 | 76.628 | 1.00 | 18.84 | C |

| | | | | | | | | | | | |
|---|---|---|---|---|---|---|---|---|---|---|---|
| ANISOU | 505 | CB | ILE A | 461 | 2628 | 2107 | 2425 | 55 | 265 | −100 | C |

| | | | | | | | | | | |
|---|---|---|---|---|---|---|---|---|---|---|
| ATOM | 506 | CG1 | ILE A | 461 | 24.586 | 17.730 | 77.448 | 1.00 | 18.90 | C |

| | | | | | | | | | | | |
|---|---|---|---|---|---|---|---|---|---|---|---|
| ANISOU | 506 | CG1 | ILE A | 461 | 2579 | 1924 | 2679 | 36 | 272 | −37 | C |

| | | | | | | | | | | |
|---|---|---|---|---|---|---|---|---|---|---|
| ATOM | 507 | CG2 | ILE A | 461 | 22.790 | 15.936 | 77.513 | 1.00 | 20.87 | C |

| | | | | | | | | | | | |
|---|---|---|---|---|---|---|---|---|---|---|---|
| ANISOU | 507 | CG2 | ILE A | 461 | 2770 | 2407 | 2752 | −70 | 467 | −178 | C |

| | | | | | | | | | | |
|---|---|---|---|---|---|---|---|---|---|---|
| ATOM | 508 | CD1 | ILE A | 461 | 25.508 | 17.178 | 78.498 | 1.00 | 20.48 | C |

| | | | | | | | | | | | |
|---|---|---|---|---|---|---|---|---|---|---|---|
| ANISOU | 508 | CD1 | ILE A | 461 | 3146 | 2476 | 2160 | −241 | 58 | −364 | C |

| | | | | | | | | | | |
|---|---|---|---|---|---|---|---|---|---|---|
| ATOM | 509 | N | GLU A | 462 | 23.211 | 14.742 | 74.214 | 1.00 | 21.71 | N |

| | | | | | | | | | | | |
|---|---|---|---|---|---|---|---|---|---|---|---|
| ANISOU | 509 | N | GLU A | 462 | 2891 | 2274 | 3082 | 88 | −116 | −169 | N |

| | | | | | | | | | | |
|---|---|---|---|---|---|---|---|---|---|---|
| ATOM | 510 | CA | GLU A | 462 | 22.502 | 13.576 | 73.646 | 1.00 | 24.28 | C |

| | | | | | | | | | | | |
|---|---|---|---|---|---|---|---|---|---|---|---|
| ANISOU | 510 | CA | GLU A | 462 | 3130 | 2683 | 3412 | −181 | −213 | −64 | C |

| | | | | | | | | | | |
|---|---|---|---|---|---|---|---|---|---|---|
| ATOM | 511 | C | GLU A | 462 | 23.526 | 12.506 | 73.263 | 1.00 | 26.65 | C |

| | | | | | | | | | | | |
|---|---|---|---|---|---|---|---|---|---|---|---|
| ANISOU | 511 | C | GLU A | 462 | 3576 | 2961 | 3588 | 78 | −124 | −114 | C |

| | | | | | | | | | | |
|---|---|---|---|---|---|---|---|---|---|---|
| ATOM | 512 | O | GLU A | 462 | 23.205 | 11.319 | 73.478 | 1.00 | 29.22 | O |

| | | | | | | | | | | | |
|---|---|---|---|---|---|---|---|---|---|---|---|
| ANISOU | 512 | O | GLU A | 462 | 3975 | 3011 | 4116 | 7 | −214 | −11 | O |

| | | | | | | | | | | |
|---|---|---|---|---|---|---|---|---|---|---|
| ATOM | 513 | CB | GLU A | 462 | 21.823 | 13.963 | 72.333 | 1.00 | 25.44 | C |

| | | | | | | | | | | | |
|---|---|---|---|---|---|---|---|---|---|---|---|
| ANISOU | 513 | CB | GLU A | 462 | 3354 | 3083 | 3228 | −14 | −77 | −138 | C |

| | | | | | | | | | | |
|---|---|---|---|---|---|---|---|---|---|---|
| ATOM | 514 | CG | GLU A | 462 | 20.526 | 14.677 | 72.576 | 1.00 | 26.27 | C |

| | | | | | | | | | | | |
|---|---|---|---|---|---|---|---|---|---|---|---|
| ANISOU | 514 | CG | GLU A | 462 | 3280 | 3410 | 3290 | 31 | 59 | −94 | C |

| | | | | | | | | | | |
|---|---|---|---|---|---|---|---|---|---|---|
| ATOM | 515 | CD | GLU A | 462 | 19.792 | 15.049 | 71.300 | 1.00 | 26.77 | C |

| | | | | | | | | | | | |
|---|---|---|---|---|---|---|---|---|---|---|---|
| ANISOU | 515 | CD | GLU A | 462 | 3197 | 3557 | 3418 | 73 | 5 | −102 | C |

| | | | | | | | | | | |
|---|---|---|---|---|---|---|---|---|---|---|
| ATOM | 516 | OE1 | GLU A | 462 | 20.493 | 15.311 | 70.292 | 1.00 | 28.84 | O |

| | | | | | | | | | | | |
|---|---|---|---|---|---|---|---|---|---|---|---|
| ANISOU | 516 | OE1 | GLU A | 462 | 3595 | 3920 | 3444 | 107 | 100 | −16 | O |

| | | | | | | | | | | |
|---|---|---|---|---|---|---|---|---|---|---|
| ATOM | 517 | OE2 | GLU A | 462 | 18.555 | 15.113 | 71.317 | 1.00 | 23.89 | O |

| | | | | | | | | | | | |
|---|---|---|---|---|---|---|---|---|---|---|---|
| ANISOU | 517 | OE2 | GLU A | 462 | 3043 | 3045 | 2989 | −182 | 76 | −328 | O |

| | | | | | | | | | | |
|---|---|---|---|---|---|---|---|---|---|---|
| ATOM | 518 | N | LYS A | 463 | 24.594 | 12.909 | 72.601 | 1.00 | 27.04 | N |

| | | | | | | | | | | | |
|---|---|---|---|---|---|---|---|---|---|---|---|
| ANISOU | 518 | N | LYS A | 463 | 3545 | 2988 | 3739 | 118 | −161 | −179 | N |

| | | | | | | | | | | |
|---|---|---|---|---|---|---|---|---|---|---|
| ATOM | 519 | CA | LYS A | 463 | 25.637 | 11.933 | 72.195 | 1.00 | 30.01 | C |

| | | | | | | | | | | | |
|---|---|---|---|---|---|---|---|---|---|---|---|
| ANISOU | 519 | CA | LYS A | 463 | 3917 | 3383 | 4101 | 288 | −71 | −280 | C |

| | | | | | | | | | | |
|---|---|---|---|---|---|---|---|---|---|---|
| ATOM | 520 | C | LYS A | 463 | 26.201 | 11.198 | 73.389 | 1.00 | 30.47 | C |

| | | | | | | | | | | | |
|---|---|---|---|---|---|---|---|---|---|---|---|
| ANISOU | 520 | C | LYS A | 463 | 4046 | 3525 | 4005 | 184 | −29 | −198 | C |

| | | | | | | | | | | |
|---|---|---|---|---|---|---|---|---|---|---|
| ATOM | 521 | O | LYS A | 463 | 26.547 | 10.001 | 73.229 | 1.00 | 31.93 | O |

| | | | | | | | | | | | |
|---|---|---|---|---|---|---|---|---|---|---|---|
| ANISOU | 521 | O | LYS A | 463 | 4310 | 3516 | 4306 | 343 | −65 | −221 | O |

| | | | | | | | | | | |
|---|---|---|---|---|---|---|---|---|---|---|
| ATOM | 522 | CB | LYS A | 463 | 26.724 | 12.649 | 71.406 | 1.00 | 32.61 | C |

| | | | | | | | | | | | |
|---|---|---|---|---|---|---|---|---|---|---|---|
| ANISOU | 522 | CB | LYS A | 463 | 4146 | 3926 | 4320 | 130 | −15 | −89 | C |

| | | | | | | | | | | |
|---|---|---|---|---|---|---|---|---|---|---|
| ATOM | 523 | CG | LYS A | 463 | 27.867 | 11.853 | 70.769 | 1.00 | 35.79 | C |

| | | | | | | | | | | | |
|---|---|---|---|---|---|---|---|---|---|---|---|
| ANISOU | 523 | CG | LYS A | 463 | 4441 | 4407 | 4751 | 236 | 97 | −107 | C |

| | | | | | | | | | | |
|---|---|---|---|---|---|---|---|---|---|---|
| ATOM | 524 | CD | LYS A | 463 | 27.330 | 11.030 | 69.607 | 1.00 | 38.85 | C |

| | | | | | | | | | | | |
|---|---|---|---|---|---|---|---|---|---|---|---|
| ANISOU | 524 | CD | LYS A | 463 | 4993 | 4924 | 4844 | −8 | −63 | −111 | C |

| | | | | | | | | | | |
|---|---|---|---|---|---|---|---|---|---|---|
| ATOM | 525 | CE | LYS A | 463 | 28.424 | 10.101 | 69.083 | 1.00 | 40.78 | C |

| | | | | | | | | | | | |
|---|---|---|---|---|---|---|---|---|---|---|---|
| ANISOU | 525 | CE | LYE A | 463 | 5246 | 5073 | 5175 | 94 | 60 | −62 | C |

| | | | | | | | | | | |
|---|---|---|---|---|---|---|---|---|---|---|
| ATOM | 526 | NZ | LYS A | 463 | 28.029 | 9.599 | 67.739 | 1.00 | 42.97 | N |

| | | | | | | | | | | | |
|---|---|---|---|---|---|---|---|---|---|---|---|
| ANISOU | 526 | NZ | LYE A | 463 | 5490 | 5561 | 5273 | −22 | −7 | −72 | N |

| | | | | | | | | | | |
|---|---|---|---|---|---|---|---|---|---|---|
| ATOM | 527 | N | SER A | 464 | 26.359 | 11.762 | 74.584 | 1.00 | 29.67 | N |

| | | | | | | | | | | | |
|---|---|---|---|---|---|---|---|---|---|---|---|
| ANISOU | 527 | N | SER A | 464 | 3934 | 3407 | 3931 | 284 | −28 | −184 | N |

| | | | | | | | | | | |
|---|---|---|---|---|---|---|---|---|---|---|
| ATOM | 528 | CA | SER A | 464 | 26.973 | 11.055 | 75.698 | 1.00 | 28.97 | C |

| | | | | | | | | | | | |
|---|---|---|---|---|---|---|---|---|---|---|---|
| ANISOU | 528 | CA | SER A | 464 | 3755 | 3459 | 3792 | 237 | 56 | −200 | C |

| | | | | | | | | | | |
|---|---|---|---|---|---|---|---|---|---|---|
| ATOM | 529 | C | SER A | 464 | 26.113 | 10.521 | 76.816 | 1.00 | 29.16 | C |

| | | | | | | | | | | | |
|---|---|---|---|---|---|---|---|---|---|---|---|
| ANISOU | 529 | C | SER A | 464 | 3700 | 3427 | 3952 | −1 | −87 | −65 | C |

| | | | | | | | | | | |
|---|---|---|---|---|---|---|---|---|---|---|
| ATOM | 530 | O | SER A | 464 | 26.582 | 10.097 | 77.901 | 1.00 | 28.24 | O |

| | | | | | | | | | | | |
|---|---|---|---|---|---|---|---|---|---|---|---|
| ANISOU | 530 | O | SER A | 464 | 3542 | 3062 | 4126 | 84 | 26 | 249 | O |

| | | | | | | | | | | |
|---|---|---|---|---|---|---|---|---|---|---|
| ATOM | 531 | CB | SER A | 464 | 28.026 | 12.035 | 76.245 | 1.00 | 29.78 | C |

| | | | | | | | | | | | |
|---|---|---|---|---|---|---|---|---|---|---|---|
| ANISOU | 531 | CB | SER A | 464 | 3809 | 3659 | 3848 | 160 | −75 | −132 | C |

| | | | | | | | | | | |
|---|---|---|---|---|---|---|---|---|---|---|
| ATOM | 532 | OG | SER A | 464 | 27.431 | 13.251 | 76.610 | 1.00 | 29.52 | O |

| | | | | | | | | | | | |
|---|---|---|---|---|---|---|---|---|---|---|---|
| ANISOU | 532 | OG | SER A | 464 | 3868 | 3550 | 3798 | 152 | −229 | 17 | O |

| | | | | | | | | | | |
|---|---|---|---|---|---|---|---|---|---|---|
| ATOM | 533 | N | THR A | 465 | 24.793 | 10.647 | 76.656 | 1.00 | 28.41 | N |

| | | | | | | | | | | | |
|---|---|---|---|---|---|---|---|---|---|---|---|
| ANISOU | 533 | N | THR A | 465 | 3630 | 3105 | 4060 | 235 | −16 | −158 | N |

| | | | | | | | | | | |
|---|---|---|---|---|---|---|---|---|---|---|
| ATOM | 534 | CA | THR A | 465 | 23.874 | 10.237 | 77.705 | 1.00 | 29.04 | C |

| | | | | | | | | | | | |
|---|---|---|---|---|---|---|---|---|---|---|---|
| ANISOU | 534 | CA | THR A | 465 | 3807 | 3263 | 3964 | 36 | −79 | −9 | C |

| | | | | | | | | | | |
|---|---|---|---|---|---|---|---|---|---|---|
| ATOM | 535 | C | THR A | 465 | 22.765 | 9.367 | 77.113 | 1.00 | 29.92 | C |

| | | | | | | | | | | | |
|---|---|---|---|---|---|---|---|---|---|---|---|
| ANISOU | 535 | C | THR A | 465 | 3866 | 3459 | 4043 | 1 | −105 | −77 | C |

| | | | | | | | | | | |
|---|---|---|---|---|---|---|---|---|---|---|
| ATOM | 536 | O | THR A | 465 | 22.110 | 9.745 | 76.147 | 1.00 | 28.27 | O |

| | | | | | | | | | | | |
|---|---|---|---|---|---|---|---|---|---|---|---|
| ANISOU | 536 | O | THR A | 465 | 3875 | 2943 | 3923 | −227 | −180 | −218 | O |

| | | | | | | | | | | |
|---|---|---|---|---|---|---|---|---|---|---|
| ATOM | 537 | CB | THR A | 465 | 23.179 | 11.425 | 78.396 | 1.00 | 28.00 | C |

| | | | | | | | | | | | |
|---|---|---|---|---|---|---|---|---|---|---|---|
| ANISOU | 537 | CB | THR A | 465 | 3390 | 3411 | 3838 | −29 | −62 | −15 | C |

| | | | | | | | | | | |
|---|---|---|---|---|---|---|---|---|---|---|
| ATOM | 538 | OG1 | THR A | 465 | 24.185 | 12.412 | 78.730 | 1.00 | 28.05 | O |

| | | | | | | | | | | | |
|---|---|---|---|---|---|---|---|---|---|---|---|
| ANISOU | 538 | OG1 | THR A | 465 | 3745 | 3194 | 3718 | −291 | 24 | 27 | O |

| | | | | | | | | | | |
|---|---|---|---|---|---|---|---|---|---|---|
| ATOM | 539 | CG2 | THR A | 465 | 22.504 | 11.050 | 79.691 | 1.00 | 28.13 | C |

| | | | | | | | | | | | |
|---|---|---|---|---|---|---|---|---|---|---|---|
| ANISOU | 539 | CG2 | THR A | 465 | 3513 | 3375 | 3800 | −22 | −136 | 134 | C |

| | | | | | | | | | | |
|---|---|---|---|---|---|---|---|---|---|---|
| ATOM | 540 | N | PRO A | 466 | 22.593 | 8.217 | 77.761 | 1.00 | 31.12 | N |

| | | | | | | | | | | | |
|---|---|---|---|---|---|---|---|---|---|---|---|
| ANISOU | 540 | N | PRO A | 466 | 3965 | 3610 | 4251 | −64 | −32 | −2 | N |

| | | | | | | | | | | |
|---|---|---|---|---|---|---|---|---|---|---|
| ATOM | 541 | CA | PRO A | 466 | 21.580 | 7.290 | 77.300 | 1.00 | 30.49 | C |

| | | | | | | | | | | | |
|---|---|---|---|---|---|---|---|---|---|---|---|
| ANISOU | 541 | CA | PRO A | 466 | 3953 | 3494 | 4138 | −32 | 38 | −10 | C |

| | | | | | | | | | | |
|---|---|---|---|---|---|---|---|---|---|---|
| ATOM | 542 | C | PRO A | 466 | 20.174 | 7.752 | 77.602 | 1.00 | 29.72 | C |

| | | | | | | | | | | | |
|---|---|---|---|---|---|---|---|---|---|---|---|
| ANISOU | 542 | C | PRO A | 466 | 3899 | 3308 | 4087 | −52 | −17 | 59 | C |

| | | | | | | | | | | |
|---|---|---|---|---|---|---|---|---|---|---|
| ATOM | 543 | O | PRO A | 466 | 19.820 | 8.418 | 78.614 | 1.00 | 27.92 | O |

| | | | | | | | | | | | |
|---|---|---|---|---|---|---|---|---|---|---|---|
| ANISOU | 543 | O | PRO A | 466 | 3477 | 3036 | 4094 | −178 | −59 | 102 | O |

| | | | | | | | | | | |
|---|---|---|---|---|---|---|---|---|---|---|
| ATOM | 544 | CB | PRO A | 466 | 21.950 | 5.982 | 77.999 | 1.00 | 31.51 | C |

| | | | | | | | | | | | |
|---|---|---|---|---|---|---|---|---|---|---|---|
| ANISOU | 544 | CB | PRO A | 466 | 4144 | 3575 | 4254 | 79 | −73 | 22 | C |

| | | | | | | | | | | |
|---|---|---|---|---|---|---|---|---|---|---|
| ATOM | 545 | CG | PRO A | 466 | 22.814 | 6.336 | 79.149 | 1.00 | 31.98 | C |

| | | | | | | | | | | | |
|---|---|---|---|---|---|---|---|---|---|---|---|
| ANISOU | 545 | CG | PRO A | 466 | 4032 | 3787 | 4330 | 45 | −85 | 92 | C |

| | | | | | | | | | | |
|---|---|---|---|---|---|---|---|---|---|---|
| ATOM | 546 | CD | PRO A | 466 | 23.338 | 7.725 | 78.931 | 1.00 | 31.87 | C |

| | | | | | | | | | | | |
|---|---|---|---|---|---|---|---|---|---|---|---|
| ANISOU | 546 | CD | PRO A | 466 | 4120 | 3768 | 4222 | 34 | 14 | 20 | C |

| | | | | | | | | | | |
|---|---|---|---|---|---|---|---|---|---|---|
| ATOM | 547 | N | ASN A | 467 | 19.317 | 7.312 | 76.673 | 1.00 | 30.01 | N |

| | | | | | | | | | | | |
|---|---|---|---|---|---|---|---|---|---|---|---|
| ANISOU | 547 | N | ASN A | 467 | 4027 | 3329 | 4047 | 175 | −47 | −98 | N |

| | | | | | | | | | | |
|---|---|---|---|---|---|---|---|---|---|---|
| ATOM | 548 | CA | ASN A | 467 | 17.882 | 7.496 | 76.691 | 1.00 | 29.27 | C |

| | | | | | | | | | | | |
|---|---|---|---|---|---|---|---|---|---|---|---|
| ANISOU | 548 | CA | ASN A | 467 | 3912 | 3172 | 4037 | 63 | 61 | −143 | C |

| | | | | | | | | | | |
|---|---|---|---|---|---|---|---|---|---|---|
| ATOM | 549 | C | ASN A | 467 | 17.543 | 8.988 | 76.884 | 1.00 | 26.77 | C |

| | | | | | | | | | | | |
|---|---|---|---|---|---|---|---|---|---|---|---|
| ANISOU | 549 | C | ASN A | 467 | 3503 | 3002 | 3667 | 35 | 184 | 47 | C |

| | | | | | | | | | | |
|---|---|---|---|---|---|---|---|---|---|---|
| ATOM | 550 | O | ASN A | 467 | 16.652 | 9.284 | 77.688 | 1.00 | 25.43 | O |

| | | | | | | | | | | | |
|---|---|---|---|---|---|---|---|---|---|---|---|
| ANISOU | 550 | O | ASN A | 467 | 3435 | 2380 | 3849 | −118 | 236 | 54 | O |

| | | | | | | | | | | |
|---|---|---|---|---|---|---|---|---|---|---|
| ATOM | 551 | CB | ASN A | 467 | 17.272 | 6.706 | 77.864 | 1.00 | 31.93 | C |

| | | | | | | | | | | | |
|---|---|---|---|---|---|---|---|---|---|---|---|
| ANISOU | 551 | CB | ASN A | 467 | 4314 | 3582 | 4235 | −106 | 8 | 34 | C |

| | | | | | | | | | | |
|---|---|---|---|---|---|---|---|---|---|---|
| ATOM | 552 | CG | ASN A | 467 | 17.717 | 5.236 | 77.845 | 1.00 | 34.30 | C |

| | | | | | | | | | | | |
|---|---|---|---|---|---|---|---|---|---|---|---|
| ANISOU | 552 | CG | ASN A | 467 | 4708 | 3733 | 4592 | −15 | 40 | 8 | C |

| | | | | | | | | | | |
|---|---|---|---|---|---|---|---|---|---|---|
| ATOM | 553 | OD1 | ASN A | 467 | 17.676 | 4.624 | 76.784 | 1.00 | 35.12 | O |

| | | | | | | | | | | | |
|---|---|---|---|---|---|---|---|---|---|---|---|
| ANISOU | 553 | OD1 | ASN A | 467 | 4909 | 3598 | 4837 | −226 | 62 | −111 | O |

| | | | | | | | | | | |
|---|---|---|---|---|---|---|---|---|---|---|
| ATOM | 554 | ND2 | ASN A | 467 | 18.208 | 4.733 | 78.956 | 1.00 | 34.98 | N |

| | | | | | | | | | | | |
|---|---|---|---|---|---|---|---|---|---|---|---|
| ANISOU | 554 | ND2 | ASN A | 467 | 4788 | 3804 | 4698 | 47 | 28 | 63 | N |

| | | | | | | | | | | |
|---|---|---|---|---|---|---|---|---|---|---|
| ATOM | 555 | N | THR A | 468 | 18.283 | 9.880 | 76.240 | 1.00 | 23.46 | N |

| | | | | | | | | | | | |
|---|---|---|---|---|---|---|---|---|---|---|---|
| ANISOU | 555 | N | THR A | 468 | 3151 | 2335 | 3429 | 284 | −80 | −151 | N |

| | | | | | | | | | | |
|---|---|---|---|---|---|---|---|---|---|---|
| ATOM | 556 | CA | THR A | 468 | 18.032 | 11.308 | 76.475 | 1.00 | 22.54 | C |

| | | | | | | | | | | | |
|---|---|---|---|---|---|---|---|---|---|---|---|
| ANISOU | 556 | CA | THR A | 468 | 3109 | 2271 | 3182 | 51 | −126 | −79 | C |

| | | | | | | | | | | |
|---|---|---|---|---|---|---|---|---|---|---|
| ATOM | 557 | C | THR A | 468 | 17.834 | 12.048 | 75.175 | 1.00 | 22.67 | C |

| | | | | | | | | | | | |
|---|---|---|---|---|---|---|---|---|---|---|---|
| ANISOU | 557 | C | THR A | 468 | 3012 | 2460 | 3143 | 121 | 26 | −73 | C |

| | | | | | | | | | | |
|---|---|---|---|---|---|---|---|---|---|---|
| ATOM | 558 | O | THR A | 468 | 18.567 | 11.809 | 74.234 | 1.00 | 24.15 | O |

| | | | | | | | | | | | |
|---|---|---|---|---|---|---|---|---|---|---|---|
| ANISOU | 558 | O | THR A | 468 | 3331 | 2680 | 3165 | 288 | 119 | −17 | O |

| | | | | | | | | | | |
|---|---|---|---|---|---|---|---|---|---|---|
| ATOM | 559 | CB | THR A | 468 | 19.280 | 11.897 | 77.200 | 1.00 | 24.18 | C |

| | | | | | | | | | | | |
|---|---|---|---|---|---|---|---|---|---|---|---|
| ANISOU | 559 | CB | THR A | 468 | 3173 | 2615 | 3398 | 5 | −171 | −105 | C |

| | | | | | | | | | | |
|---|---|---|---|---|---|---|---|---|---|---|
| ATOM | 560 | OG1 | THR A | 468 | 19.321 | 11.404 | 78.527 | 1.00 | 24.77 | O |

| | | | | | | | | | | | |
|---|---|---|---|---|---|---|---|---|---|---|---|
| ANISOU | 560 | OG1 | THR A | 468 | 3365 | 2594 | 3454 | −136 | −157 | −76 | O |

| | | | | | | | | | | |
|---|---|---|---|---|---|---|---|---|---|---|
| ATOM | 561 | CG2 | THR A | 468 | 19.156 | 13.431 | 77.335 | 1.00 | 25.20 | C |

| | | | | | | | | | | | |
|---|---|---|---|---|---|---|---|---|---|---|---|
| ANISOU | 561 | CG2 | THR A | 468 | 3570 | 2608 | 3397 | 38 | −89 | −131 | C |

| | | | | | | | | | | |
|---|---|---|---|---|---|---|---|---|---|---|
| ATOM | 562 | N | VAL A | 469 | 16.862 | 12.978 | 75.145 | 1.00 | 20.11 | N |

| | | | | | | | | | | | |
|---|---|---|---|---|---|---|---|---|---|---|---|
| ANISOU | 562 | N | VAL A | 469 | 2757 | 2167 | 2717 | 63 | −160 | 1 | N |

| | | | | | | | | | | |
|---|---|---|---|---|---|---|---|---|---|---|
| ATOM | 563 | CA | VAL A | 469 | 16.715 | 13.872 | 74.015 | 1.00 | 19.77 | C |

| | | | | | | | | | | | |
|---|---|---|---|---|---|---|---|---|---|---|---|
| ANISOU | 563 | CA | VAL A | 469 | 2753 | 2152 | 2605 | 20 | −89 | −120 | C |

| | | | | | | | | | | |
|---|---|---|---|---|---|---|---|---|---|---|
| ATOM | 564 | C | VAL A | 469 | 16.977 | 15.273 | 74.579 | 1.00 | 18.75 | C |

| | | | | | | | | | | | |
|---|---|---|---|---|---|---|---|---|---|---|---|
| ANISOU | 564 | C | VAL A | 469 | 2545 | 2095 | 2485 | −110 | −69 | −42 | C |

| | | | | | | | | | | |
|---|---|---|---|---|---|---|---|---|---|---|
| ATOM | 565 | O | VAL A | 469 | 16.511 | 15.555 | 75.702 | 1.00 | 19.29 | O |

| | | | | | | | | | | | |
|---|---|---|---|---|---|---|---|---|---|---|---|
| ANISOU | 565 | O | VAL A | 469 | 2525 | 2117 | 2687 | −104 | 148 | −1 | O |

| | | | | | | | | | | |
|---|---|---|---|---|---|---|---|---|---|---|
| ATOM | 566 | CB | VAL A | 469 | 15.315 | 13.826 | 73.386 | 1.00 | 18.35 | C |

| | | | | | | | | | | | |
|---|---|---|---|---|---|---|---|---|---|---|---|
| ANISOU | 566 | CB | VAL A | 469 | 2601 | 1953 | 2417 | −51 | −76 | 11 | C |

| | | | | | | | | | | |
|---|---|---|---|---|---|---|---|---|---|---|
| ATOM | 567 | CG1 | VAL A | 469 | 14.952 | 15.012 | 72.498 | 1.00 | 18.07 | C |

| | | | | | | | | | | | |
|---|---|---|---|---|---|---|---|---|---|---|---|
| ANISOU | 567 | CG1 | VAL A | 469 | 2532 | 2076 | 2257 | 33 | 36 | 40 | C |

| | | | | | | | | | | |
|---|---|---|---|---|---|---|---|---|---|---|
| ATOM | 568 | CD2 | VAL A | 469 | 15.243 | 12.507 | 72.570 | 1.00 | 20.95 | C |

| | | | | | | | | | | | |
|---|---|---|---|---|---|---|---|---|---|---|---|
| ANISOU | 568 | CG2 | VAL A | 469 | 3064 | 2204 | 2694 | −68 | 20 | −216 | C |

| | | | | | | | | | | |
|---|---|---|---|---|---|---|---|---|---|---|
| ATOM | 569 | N | ALA A | 470 | 17.707 | 16.045 | 73.809 | 1.00 | 17.32 | N |

| | | | | | | | | | | | |
|---|---|---|---|---|---|---|---|---|---|---|---|
| ANISOU | 569 | N | ALA A | 470 | 2214 | 1828 | 2540 | 56 | 5 | −187 | N |

| | | | | | | | | | | |
|---|---|---|---|---|---|---|---|---|---|---|
| ATOM | 570 | CA | ALA A | 470 | 17.914 | 17.453 | 74.210 | 1.00 | 17.85 | C |

| | | | | | | | | | | | |
|---|---|---|---|---|---|---|---|---|---|---|---|
| ANISOU | 570 | CA | ALA A | 470 | 2268 | 1788 | 2725 | −72 | −6 | −104 | C |

| | | | | | | | | | | |
|---|---|---|---|---|---|---|---|---|---|---|
| ATOM | 571 | C | ALA A | 470 | 17.602 | 18.261 | 72.963 | 1.00 | 17.99 | C |

| | | | | | | | | | | | |
|---|---|---|---|---|---|---|---|---|---|---|---|
| ANISOU | 571 | C | ALA A | 470 | 2199 | 2066 | 2569 | −9 | −7 | −162 | C |

| | | | | | | | | | | |
|---|---|---|---|---|---|---|---|---|---|---|
| ATOM | 572 | O | ALA A | 470 | 18.336 | 18.213 | 71.970 | 1.00 | 20.67 | O |

| | | | | | | | | | | | |
|---|---|---|---|---|---|---|---|---|---|---|---|
| ANISOU | 572 | O | ALA A | 470 | 2565 | 2411 | 2878 | 174 | 78 | 105 | O |

| | | | | | | | | | | |
|---|---|---|---|---|---|---|---|---|---|---|
| ATOM | 573 | CB | ALA A | 470 | 19.318 | 17.689 | 74.748 | 1.00 | 19.31 | C |

| | | | | | | | | | | | |
|---|---|---|---|---|---|---|---|---|---|---|---|
| ANISOU | 573 | CB | ALA A | 470 | 2174 | 2315 | 2847 | 9 | 93 | −235 | C |

| | | | | | | | | | | |
|---|---|---|---|---|---|---|---|---|---|---|
| ATOM | 574 | N | PHE A | 471 | 16.437 | 18.953 | 72.987 | 1.00 | 17.19 | N |

| | | | | | | | | | | | |
|---|---|---|---|---|---|---|---|---|---|---|---|
| ANISOU | 574 | N | PHE A | 471 | 2401 | 1730 | 2401 | −122 | 90 | 57 | N |

| | | | | | | | | | | |
|---|---|---|---|---|---|---|---|---|---|---|
| ATOM | 575 | CA | PHE A | 471 | 16.057 | 19.708 | 71.814 | 1.00 | 18.27 | C |

| | | | | | | | | | | | |
|---|---|---|---|---|---|---|---|---|---|---|---|
| ANISOU | 575 | CA | PHE A | 471 | 2217 | 2211 | 2515 | 39 | −58 | 42 | C |

| | | | | | | | | | | |
|---|---|---|---|---|---|---|---|---|---|---|
| ATOM | 576 | C | PHE A | 471 | 16.863 | 21.017 | 71.716 | 1.00 | 17.18 | C |

| | | | | | | | | | | | |
|---|---|---|---|---|---|---|---|---|---|---|---|
| ANISOU | 576 | C | PHE A | 471 | 2178 | 2138 | 2210 | 54 | −64 | 60 | C |

| | | | | | | | | | | |
|---|---|---|---|---|---|---|---|---|---|---|
| ATOM | 577 | O | PHE A | 471 | 17.316 | 21.515 | 72.758 | 1.00 | 18.28 | O |

| | | | | | | | | | | | |
|---|---|---|---|---|---|---|---|---|---|---|---|
| ANISOU | 577 | O | PHE A | 471 | 2178 | 2274 | 2492 | 28 | −21 | −128 | O |

| | | | | | | | | | | |
|---|---|---|---|---|---|---|---|---|---|---|
| ATOM | 578 | CB | PHE A | 471 | 14.559 | 20.000 | 71.881 | 1.00 | 19.21 | C |

| | | | | | | | | | | | |
|---|---|---|---|---|---|---|---|---|---|---|---|
| ANISOU | 578 | CB | PHE A | 471 | 2311 | 2380 | 2608 | 106 | −23 | −80 | C |

| | | | | | | | | | | |
|---|---|---|---|---|---|---|---|---|---|---|
| ATOM | 579 | CG | PHE A | 471 | 13.641 | 18.855 | 71.543 | 1.00 | 20.35 | C |

| | | | | | | | | | | | |
|---|---|---|---|---|---|---|---|---|---|---|---|
| ANISOU | 579 | CG | PHE A | 471 | 2629 | 2280 | 2821 | 61 | −11 | −59 | C |

| | | | | | | | | | | |
|---|---|---|---|---|---|---|---|---|---|---|
| ATOM | 580 | CD1 | PHE A | 471 | 12.771 | 18.333 | 72.479 | 1.00 | 21.16 | C |

| | | | | | | | | | | | |
|---|---|---|---|---|---|---|---|---|---|---|---|
| ANISOU | 580 | CD1 | PHE A | 471 | 2677 | 2448 | 2917 | −146 | 3 | −85 | C |

| | | | | | | | | | | |
|---|---|---|---|---|---|---|---|---|---|---|
| ATOM | 581 | CD2 | PHE A | 471 | 13.685 | 18.342 | 70.245 | 1.00 | 20.43 | C |

| | | | | | | | | | | | |
|---|---|---|---|---|---|---|---|---|---|---|---|
| ANISOU | 581 | CD2 | PHE A | 471 | 2617 | 2396 | 2750 | −57 | −183 | −47 | C |

| | | | | | | | | | | |
|---|---|---|---|---|---|---|---|---|---|---|
| ATOM | 582 | CE1 | PHE A | 471 | 11.907 | 17.276 | 72.146 | 1.00 | 21.30 | C |

| | | | | | | | | | | | |
|---|---|---|---|---|---|---|---|---|---|---|---|
| ANISOU | 582 | CE1 | PHE A | 471 | 2707 | 2495 | 2891 | −177 | −28 | −22 | C |

| | | | | | | | | | | |
|---|---|---|---|---|---|---|---|---|---|---|
| ATOM | 583 | CE2 | PHE A | 471 | 12.813 | 17.296 | 69.904 | 1.00 | 21.06 | C |

| | | | | | | | | | | | |
|---|---|---|---|---|---|---|---|---|---|---|---|
| ANISOU | 583 | CE2 | PHE A | 471 | 2543 | 2614 | 2844 | −163 | −185 | 56 | C |

| | | | | | | | | | | |
|---|---|---|---|---|---|---|---|---|---|---|
| ATOM | 584 | CZ | PHE A | 471 | 11.963 | 16.779 | 70.860 | 1.00 | 22.63 | C |

| | | | | | | | | | | | |
|---|---|---|---|---|---|---|---|---|---|---|---|
| ANISOU | 584 | CZ | PHE A | 471 | 2965 | 2743 | 2890 | −119 | −27 | 41 | C |

| | | | | | | | | | | |
|---|---|---|---|---|---|---|---|---|---|---|
| ATOM | 585 | N | ASN A | 472 | 16.923 | 21.556 | 70.513 | 1.00 | 16.38 | N |

| | | | | | | | | | | | |
|---|---|---|---|---|---|---|---|---|---|---|---|
| ANISOU | 585 | N | ASN A | 472 | 2110 | 1982 | 2133 | 150 | 90 | −18 | N |

| | | | | | | | | | | |
|---|---|---|---|---|---|---|---|---|---|---|
| ATOM | 586 | CA | ASN A | 472 | 17.469 | 22.913 | 70.358 | 1.00 | 17.74 | C |

| | | | | | | | | | | | |
|---|---|---|---|---|---|---|---|---|---|---|---|
| ANISOU | 586 | CA | ASN A | 472 | 2332 | 2070 | 2338 | 98 | 45 | −29 | C |

| | | | | | | | | | | |
|---|---|---|---|---|---|---|---|---|---|---|
| ATOM | 587 | C | ASN A | 472 | 16.404 | 23.867 | 70.967 | 1.00 | 17.09 | C |

| | | | | | | | | | | | |
|---|---|---|---|---|---|---|---|---|---|---|---|
| ANISOU | 587 | C | ASN A | 472 | 2307 | 2054 | 2132 | 30 | 23 | 50 | C |

| | | | | | | | | | | |
|---|---|---|---|---|---|---|---|---|---|---|
| ATOM | 588 | O | ASN A | 472 | 15.276 | 23.490 | 71.257 | 1.00 | 17.50 | O |

| | | | | | | | | | | | |
|---|---|---|---|---|---|---|---|---|---|---|---|
| ANISOU | 588 | O | ASN A | 472 | 2375 | 2234 | 2042 | 46 | −56 | −173 | O |

| | | | | | | | | | | |
|---|---|---|---|---|---|---|---|---|---|---|
| ATOM | 589 | CB | ASN A | 472 | 17.796 | 23.224 | 68.913 | 1.00 | 19.19 | C |

| | | | | | | | | | | | |
|---|---|---|---|---|---|---|---|---|---|---|---|
| ANISOU | 589 | CB | ASN A | 472 | 2484 | 2441 | 2365 | −38 | −53 | 73 | C |

| | | | | | | | | | | |
|---|---|---|---|---|---|---|---|---|---|---|
| ATOM | 590 | CG | ASN A | 472 | 16.619 | 23.180 | 67.945 | 1.00 | 20.81 | C |

| | | | | | | | | | | | |
|---|---|---|---|---|---|---|---|---|---|---|---|
| ANISOU | 590 | CG | ASN A | 472 | 2619 | 2728 | 2560 | 19 | −130 | −20 | C |

| | | | | | | | | | | |
|---|---|---|---|---|---|---|---|---|---|---|
| ATOM | 591 | OD1 | ASN A | 472 | 15.566 | 23.752 | 68.237 | 1.00 | 18.12 | O |

| | | | | | | | | | | | |
|---|---|---|---|---|---|---|---|---|---|---|---|
| ANISOU | 591 | OD1 | ASN A | 472 | 2304 | 2445 | 2136 | −94 | −235 | −89 | O |

| | | | | | | | | | | |
|---|---|---|---|---|---|---|---|---|---|---|
| ATOM | 592 | ND2 | ASN A | 472 | 16.854 | 22.512 | 66.807 | 1.00 | 21.41 | N |

| | | | | | | | | | | | |
|---|---|---|---|---|---|---|---|---|---|---|---|
| ANISOU | 592 | ND2 | ASN A | 472 | 2935 | 2648 | 2554 | −74 | −82 | −8 | N |

| | | | | | | | | | | |
|---|---|---|---|---|---|---|---|---|---|---|
| ATOM | 593 | N | SER A | 473 | 16.766 | 25.165 | 71.034 | 1.00 | 16.78 | N |

| | | | | | | | | | | | |
|---|---|---|---|---|---|---|---|---|---|---|---|
| ANISOU | 593 | N | SER A | 473 | 2316 | 1952 | 2107 | 176 | −76 | −71 | N |

| | | | | | | | | | | |
|---|---|---|---|---|---|---|---|---|---|---|
| ATOM | 594 | CA | SER A | 473 | 15.871 | 26.120 | 71.671 | 1.00 | 16.59 | C |

| | | | | | | | | | | | |
|---|---|---|---|---|---|---|---|---|---|---|---|
| ANISOU | 594 | CA | SER A | 473 | 2355 | 1909 | 2039 | 107 | −19 | −67 | C |

| | | | | | | | | | | |
|---|---|---|---|---|---|---|---|---|---|---|
| ATOM | 595 | C | SER A | 473 | 14.662 | 26.506 | 70.821 | 1.00 | 17.79 | C |

| | | | | | | | | | | | |
|---|---|---|---|---|---|---|---|---|---|---|---|
| ANISOU | 595 | C | SER A | 473 | 2307 | 2177 | 2274 | 78 | −38 | −65 | C |

| | | | | | | | | | | |
|---|---|---|---|---|---|---|---|---|---|---|
| ATOM | 596 | O | SER A | 473 | 13.748 | 27.143 | 71.392 | 1.00 | 19.45 | O |

| | | | | | | | | | | | |
|---|---|---|---|---|---|---|---|---|---|---|---|
| ANISOU | 596 | O | SER A | 473 | 2484 | 2502 | 2403 | 232 | −97 | 93 | O |

| | | | | | | | | | | |
|---|---|---|---|---|---|---|---|---|---|---|
| ATOM | 597 | CB | SER A | 473 | 16.643 | 27.408 | 72.032 | 1.00 | 17.18 | C |

| | | | | | | | | | | | |
|---|---|---|---|---|---|---|---|---|---|---|---|
| ANISOU | 597 | CB | SER A | 473 | 2580 | 1755 | 2191 | 57 | −16 | 40 | C |

| | | | | | | | | | | |
|---|---|---|---|---|---|---|---|---|---|---|
| ATOM | 598 | OG | SER A | 473 | 17.266 | 27.936 | 70.895 | 1.00 | 19.85 | O |

| | | | | | | | | | | | |
|---|---|---|---|---|---|---|---|---|---|---|---|
| ANISOU | 598 | OG | SER A | 473 | 2623 | 2238 | 2683 | 40 | 392 | −114 | O |

| | | | | | | | | | | |
|---|---|---|---|---|---|---|---|---|---|---|
| ATOM | 599 | N | PHE A | 474 | 14.636 | 26.157 | 69.546 | 1.00 | 17.53 | N |

| | | | | | | | | | | | |
|---|---|---|---|---|---|---|---|---|---|---|---|
| ANISOU | 599 | N | PHE A | 474 | 2285 | 2065 | 2311 | −36 | −203 | 6 | N |

| | | | | | | | | | | |
|---|---|---|---|---|---|---|---|---|---|---|
| ATOM | 600 | CA | PHE A | 474 | 13.496 | 26.490 | 68.688 | 1.00 | 18.89 | C |

| | | | | | | | | | | | |
|---|---|---|---|---|---|---|---|---|---|---|---|
| ANISOU | 600 | CA | PHE A | 474 | 2420 | 2359 | 2398 | 120 | −76 | 130 | C |

| | | | | | | | | | | |
|---|---|---|---|---|---|---|---|---|---|---|
| ATOM | 601 | C | PHE A | 474 | 12.321 | 25.582 | 69.025 | 1.00 | 18.64 | C |

| | | | | | | | | | | | |
|---|---|---|---|---|---|---|---|---|---|---|---|
| ANISOU | 601 | C | PHE A | 474 | 2454 | 2318 | 2310 | 58 | −97 | 35 | C |

| | | | | | | | | | | |
|---|---|---|---|---|---|---|---|---|---|---|
| ATOM | 602 | O | PHE A | 474 | 11.178 | 25.908 | 68.718 | 1.00 | 18.85 | O |

| | | | | | | | | | | | |
|---|---|---|---|---|---|---|---|---|---|---|---|
| ANISOU | 602 | O | PHE A | 474 | 2487 | 2253 | 2422 | 86 | −73 | 114 | O |

| | | | | | | | | | | |
|---|---|---|---|---|---|---|---|---|---|---|
| ATOM | 603 | CB | PHE A | 474 | 13.879 | 26.444 | 67.187 | 1.00 | 19.19 | C |

| | | | | | | | | | | | |
|---|---|---|---|---|---|---|---|---|---|---|---|
| ANISOU | 603 | CB | PHE A | 474 | 2443 | 2478 | 2371 | −35 | 12 | 17 | C |

| | | | | | | | | | | |
|---|---|---|---|---|---|---|---|---|---|---|
| ATOM | 604 | CG | PHE A | 474 | 14.952 | 27.483 | 66.871 | 1.00 | 19.51 | C |

| | | | | | | | | | | | |
|---|---|---|---|---|---|---|---|---|---|---|---|
| ANISOU | 604 | CG | PHE A | 474 | 2439 | 2540 | 2434 | 19 | −11 | 68 | C |

| | | | | | | | | | | |
|---|---|---|---|---|---|---|---|---|---|---|
| ATOM | 605 | CD1 | PHE A | 474 | 16.249 | 27.055 | 66.629 | 1.00 | 19.12 | C |

| | | | | | | | | | | | |
|---|---|---|---|---|---|---|---|---|---|---|---|
| ANISOU | 605 | CD1 | PHE A | 474 | 2375 | 2879 | 2009 | −82 | −17 | 110 | C |

| | | | | | | | | | | |
|---|---|---|---|---|---|---|---|---|---|---|
| ATOM | 606 | CD2 | PHE A | 474 | 14.648 | 28.842 | 66.851 | 1.00 | 19.28 | C |

| | | | | | | | | | | | |
|---|---|---|---|---|---|---|---|---|---|---|---|
| ANISOU | 606 | CD2 | PHE A | 474 | 2511 | 2535 | 2281 | −78 | −37 | 91 | C |

| | | | | | | | | | | |
|---|---|---|---|---|---|---|---|---|---|---|
| ATOM | 607 | CE1 | PHE A | 474 | 17.266 | 27.963 | 66.372 | 1.00 | 20.03 | C |

| | | | | | | | | | | | |
|---|---|---|---|---|---|---|---|---|---|---|---|
| ANISOU | 607 | CE1 | PHE A | 474 | 2701 | 2756 | 2152 | −110 | −118 | 93 | C |

| | | | | | | | | | | |
|---|---|---|---|---|---|---|---|---|---|---|
| ATOM | 608 | CE2 | PHE A | 474 | 15.666 | 29.780 | 66.589 | 1.00 | 19.86 | C |

| | | | | | | | | | | | |
|---|---|---|---|---|---|---|---|---|---|---|---|
| ANISOU | 608 | CE2 | PHE A | 474 | 2670 | 2568 | 2307 | −146 | 53 | −58 | C |

| | | | | | | | | | | |
|---|---|---|---|---|---|---|---|---|---|---|
| ATOM | 609 | CZ | PHE A | 474 | 16.950 | 29.308 | 66.353 | 1.00 | 20.70 | C |

| | | | | | | | | | | | |
|---|---|---|---|---|---|---|---|---|---|---|---|
| ANISOU | 609 | CZ | PHE A | 474 | 2784 | 2814 | 2266 | −33 | −8 | −45 | C |

| | | | | | | | | | | |
|---|---|---|---|---|---|---|---|---|---|---|
| ATOM | 610 | N | PHE A | 475 | 12.562 | 24.442 | 69.714 | 1.00 | 16.55 | N |

| | | | | | | | | | | | |
|---|---|---|---|---|---|---|---|---|---|---|---|
| ANISOU | 610 | N | PHE A | 475 | 2100 | 2232 | 1957 | −126 | −153 | 28 | N |

| | | | | | | | | | | |
|---|---|---|---|---|---|---|---|---|---|---|
| ATOM | 611 | CA | PHE A | 475 | 11.468 | 23.564 | 70.146 | 1.00 | 18.34 | C |

| | | | | | | | | | | | |
|---|---|---|---|---|---|---|---|---|---|---|---|
| ANISOU | 611 | CA | PHE A | 475 | 2388 | 2210 | 2369 | −45 | 55 | 46 | C |

| | | | | | | | | | | |
|---|---|---|---|---|---|---|---|---|---|---|
| ATOM | 612 | C | PHE A | 475 | 10.565 | 24.335 | 71.115 | 1.00 | 17.97 | C |

| | | | | | | | | | | | |
|---|---|---|---|---|---|---|---|---|---|---|---|
| ANISOU | 612 | C | PHE A | 475 | 2187 | 2295 | 2344 | 30 | 35 | 121 | C |

| | | | | | | | | | | |
|---|---|---|---|---|---|---|---|---|---|---|
| ATOM | 613 | O | PHE A | 475 | 9.345 | 24.419 | 70.846 | 1.00 | 19.98 | O |

| | | | | | | | | | | | |
|---|---|---|---|---|---|---|---|---|---|---|---|
| ANISOU | 613 | O | PHE A | 475 | 2286 | 2329 | 2977 | 94 | 121 | 39 | O |

| | | | | | | | | | | |
|---|---|---|---|---|---|---|---|---|---|---|
| ATOM | 614 | CB | PHE A | 475 | 12.079 | 22.331 | 70.829 | 1.00 | 18.66 | C |

| | | | | | | | | | | | |
|---|---|---|---|---|---|---|---|---|---|---|---|
| ANISOU | 614 | CB | PHE A | 475 | 2354 | 2382 | 2355 | −28 | 14 | 191 | C |

| | | | | | | | | | | |
|---|---|---|---|---|---|---|---|---|---|---|
| ATOM | 615 | CG | PHE A | 475 | 11.044 | 21.462 | 71.505 | 1.00 | 18.46 | C |

| | | | | | | | | | | | |
|---|---|---|---|---|---|---|---|---|---|---|---|
| ANISOU | 615 | CG | PHE A | 475 | 2325 | 2246 | 2444 | −47 | 44 | 54 | C |

| | | | | | | | | | | |
|---|---|---|---|---|---|---|---|---|---|---|
| ATOM | 616 | CD1 | PHE A | 475 | 10.398 | 20.479 | 70.778 | 1.00 | 18.82 | C |

| | | | | | | | | | | | |
|---|---|---|---|---|---|---|---|---|---|---|---|
| ANISOU | 616 | CD1 | PHE A | 475 | 2371 | 2366 | 2413 | −73 | −75 | 83 | C |

| | | | | | | | | | | |
|---|---|---|---|---|---|---|---|---|---|---|
| ATOM | 617 | CD2 | PHE A | 475 | 10.743 | 21.630 | 72.848 | 1.00 | 17.98 | C |

| | | | | | | | | | | | |
|---|---|---|---|---|---|---|---|---|---|---|---|
| ANISOU | 617 | CD2 | PHE A | 475 | 2236 | 2305 | 2292 | −1 | −148 | 64 | C |

| | | | | | | | | | | |
|---|---|---|---|---|---|---|---|---|---|---|
| ATOM | 618 | CE1 | PHE A | 475 | 9.431 | 19.690 | 71.409 | 1.00 | 19.74 | C |

| | | | | | | | | | | | |
|---|---|---|---|---|---|---|---|---|---|---|---|
| ANISOU | 618 | CE1 | PHE A | 475 | 2476 | 2360 | 2663 | −130 | 59 | 97 | C |

| | | | | | | | | | | |
|---|---|---|---|---|---|---|---|---|---|---|
| ATOM | 619 | CE2 | PHE A | 475 | 9.788 | 20.846 | 73.497 | 1.00 | 18.76 | C |

| | | | | | | | | | | | |
|---|---|---|---|---|---|---|---|---|---|---|---|
| ANISOU | 619 | CE2 | PHE A | 475 | 2431 | 2273 | 2422 | −103 | −178 | 8 | C |

| | | | | | | | | | | |
|---|---|---|---|---|---|---|---|---|---|---|
| ATOM | 620 | CZ | PHE A | 475 | 9.122 | 19.856 | 72.746 | 1.00 | 19.61 | C |

| | | | | | | | | | | | |
|---|---|---|---|---|---|---|---|---|---|---|---|
| ANISOU | 620 | CZ | PHE A | 475 | 2451 | 2401 | 2600 | −157 | −176 | −102 | C |

| | | | | | | | | | | |
|---|---|---|---|---|---|---|---|---|---|---|
| ATOM | 621 | N | TYR A | 476 | 11.138 | 24.930 | 72.150 | 1.00 | 17.77 | N |

| | | | | | | | | | | | |
|---|---|---|---|---|---|---|---|---|---|---|---|
| ANISOU | 621 | N | TYR A | 476 | 2102 | 2192 | 2457 | 104 | 134 | −19 | N |

| | | | | | | | | | | |
|---|---|---|---|---|---|---|---|---|---|---|
| ATOM | 622 | CA | TYR A | 476 | 10.241 | 25.668 | 73.065 | 1.00 | 18.09 | C |

| | | | | | | | | | | | |
|---|---|---|---|---|---|---|---|---|---|---|---|
| ANISOU | 622 | CA | TYR A | 476 | 2260 | 2271 | 2341 | 66 | −16 | −168 | C |

| | | | | | | | | | | |
|---|---|---|---|---|---|---|---|---|---|---|
| ATOM | 623 | C | TYR A | 476 | 9.597 | 26.854 | 72.351 | 1.00 | 19.49 | C |

| | | | | | | | | | | | |
|---|---|---|---|---|---|---|---|---|---|---|---|
| ANISOU | 623 | C | TYR A | 476 | 2159 | 2473 | 2775 | 29 | −52 | −8 | C |

| | | | | | | | | | | |
|---|---|---|---|---|---|---|---|---|---|---|
| ATOM | 624 | O | TYR A | 476 | 8.405 | 27.135 | 72.620 | 1.00 | 19.77 | O |

| | | | | | | | | | | | |
|---|---|---|---|---|---|---|---|---|---|---|---|
| ANISOU | 624 | O | TYR A | 476 | 2355 | 2259 | 2899 | 66 | −113 | −111 | O |

| | | | | | | | | | | |
|---|---|---|---|---|---|---|---|---|---|---|
| ATOM | 625 | CB | TYR A | 476 | 10.971 | 26.011 | 74.409 | 1.00 | 18.95 | C |

| | | | | | | | | | | | |
|---|---|---|---|---|---|---|---|---|---|---|---|
| ANISOU | 625 | CB | TYR A | 476 | 2243 | 2519 | 2438 | −63 | −169 | −63 | C |

| | | | | | | | | | | |
|---|---|---|---|---|---|---|---|---|---|---|
| ATOM | 626 | CG | TYR A | 476 | 9.879 | 26.577 | 75.298 | 1.00 | 19.83 | C |

| | | | | | | | | | | | |
|---|---|---|---|---|---|---|---|---|---|---|---|
| ANISOU | 626 | CG | TYR A | 476 | 2111 | 2665 | 2760 | 40 | −156 | −62 | C |

| | | | | | | | | | | |
|---|---|---|---|---|---|---|---|---|---|---|
| ATOM | 627 | CD1 | TYR A | 476 | 8.936 | 25.709 | 75.867 | 1.00 | 22.22 | C |

| | | | | | | | | | | | |
|---|---|---|---|---|---|---|---|---|---|---|---|
| ANISOU | 627 | CD1 | TYR A | 476 | 2516 | 2642 | 3285 | 70 | 98 | 119 | C |

| | | | | | | | | | | |
|---|---|---|---|---|---|---|---|---|---|---|
| ATOM | 628 | CD2 | TYR A | 476 | 9.698 | 27.938 | 75.396 | 1.00 | 20.04 | C |

| | | | | | | | | | | | |
|---|---|---|---|---|---|---|---|---|---|---|---|
| ANISOU | 628 | CD2 | TYR A | 476 | 2213 | 2657 | 2745 | 11 | 7 | −159 | C |

| | | | | | | | | | | |
|---|---|---|---|---|---|---|---|---|---|---|
| ATOM | 629 | CE1 | TYR A | 476 | 7.866 | 26.193 | 76.602 | 1.00 | 24.48 | C |

| | | | | | | | | | | | |
|---|---|---|---|---|---|---|---|---|---|---|---|
| ANISOU | 629 | CE1 | TYR A | 476 | 2860 | 3006 | 3436 | −1 | 235 | −49 | C |

| | | | | | | | | | | |
|---|---|---|---|---|---|---|---|---|---|---|
| ATOM | 630 | CE2 | TYR A | 476 | 8.589 | 28.431 | 76.118 | 1.00 | 21.46 | C |

| | | | | | | | | | | | |
|---|---|---|---|---|---|---|---|---|---|---|---|
| ANISOU | 630 | CE2 | TYR A | 476 | 2086 | 2907 | 3159 | 164 | 94 | −102 | C |

| | | | | | | | | | | |
|---|---|---|---|---|---|---|---|---|---|---|
| ATOM | 631 | CZ | TYR A | 476 | 7.725 | 27.548 | 76.737 | 1.00 | 23.80 | C |

| | | | | | | | | | | | |
|---|---|---|---|---|---|---|---|---|---|---|---|
| ANISOU | 631 | CZ | TYR A | 476 | 2729 | 2939 | 3374 | 175 | 209 | 20 | C |

| | | | | | | | | | | |
|---|---|---|---|---|---|---|---|---|---|---|
| ATOM | 632 | OH | TYR A | 476 | 6.631 | 28.072 | 77.419 | 1.00 | 25.08 | O |

| | | | | | | | | | | | |
|---|---|---|---|---|---|---|---|---|---|---|---|
| ANISOU | 632 | OH | TYR A | 476 | 2350 | 3420 | 3759 | 44 | 203 | −33 | O |

| | | | | | | | | | | |
|---|---|---|---|---|---|---|---|---|---|---|
| ATOM | 633 | N | THR A | 477 | 10.309 | 27.560 | 71.485 | 1.00 | 21.50 | N |

| | | | | | | | | | | | |
|---|---|---|---|---|---|---|---|---|---|---|---|
| ANISOU | 633 | N | THR A | 477 | 2740 | 2648 | 2781 | 116 | −148 | 243 | N |

| | | | | | | | | | | |
|---|---|---|---|---|---|---|---|---|---|---|
| ATOM | 634 | CA | THR A | 477 | 9.673 | 28.679 | 70.757 | 1.00 | 21.75 | C |

| | | | | | | | | | | | |
|---|---|---|---|---|---|---|---|---|---|---|---|
| ANISON | 634 | CA | THR A | 477 | 2708 | 2534 | 3024 | 90 | −163 | 172 | C |

| | | | | | | | | | | |
|---|---|---|---|---|---|---|---|---|---|---|
| ATOM | 635 | C | THR A | 477 | 8.402 | 28.201 | 70.043 | 1.00 | 21.93 | C |

| | | | | | | | | | | | |
|---|---|---|---|---|---|---|---|---|---|---|---|
| ANISOU | 635 | C | THR A | 477 | 2741 | 2723 | 2869 | −53 | −41 | 4 | C |

| | | | | | | | | | | |
|---|---|---|---|---|---|---|---|---|---|---|
| ATOM | 636 | O | THR A | 477 | 7.329 | 28.815 | 70.137 | 1.00 | 23.65 | O |

| | | | | | | | | | | | |
|---|---|---|---|---|---|---|---|---|---|---|---|
| ANISOU | 636 | O | THR A | 477 | 2810 | 2873 | 3303 | −22 | −11 | 57 | O |

| | | | | | | | | | | |
|---|---|---|---|---|---|---|---|---|---|---|
| ATOM | 637 | CB | THR A | 477 | 10.626 | 29.228 | 69.682 | 1.00 | 23.32 | C |

| | | | | | | | | | | | |
|---|---|---|---|---|---|---|---|---|---|---|---|
| ANISOU | 637 | CB | THR A | 477 | 2839 | 2850 | 3170 | 37 | −57 | 123 | C |

| | | | | | | | | | | |
|---|---|---|---|---|---|---|---|---|---|---|
| ATOM | 638 | OG1 | THR A | 477 | 11.813 | 29.668 | 70.370 | 1.00 | 23.83 | O |

| | | | | | | | | | | | |
|---|---|---|---|---|---|---|---|---|---|---|---|
| ANISOU | 638 | OG1 | THR A | 477 | 2822 | 2740 | 3491 | 87 | −217 | 280 | O |

| | | | | | | | | | | |
|---|---|---|---|---|---|---|---|---|---|---|
| ATOM | 639 | CG2 | THR A | 477 | 9.959 | 30.425 | 69.016 | 1.00 | 22.98 | C |

| | | | | | | | | | | | |
|---|---|---|---|---|---|---|---|---|---|---|---|
| ANISOU | 639 | CD2 | THR A | 477 | 2878 | 2790 | 3064 | −105 | −140 | 161 | C |

| | | | | | | | | | | |
|---|---|---|---|---|---|---|---|---|---|---|
| ATOM | 640 | N | ASN A | 478 | 8.506 | 27.135 | 69.257 | 1.00 | 19.70 | N |

| | | | | | | | | | | | |
|---|---|---|---|---|---|---|---|---|---|---|---|
| ANISOU | 640 | N | ASN A | 478 | 2369 | 2595 | 2522 | 63 | 70 | 184 | N |

| | | | | | | | | | | |
|---|---|---|---|---|---|---|---|---|---|---|
| ATOM | 641 | CA | ASN A | 478 | 7.359 | 26.589 | 68.548 | 1.00 | 21.28 | C |

| | | | | | | | | | | | |
|---|---|---|---|---|---|---|---|---|---|---|---|
| ANISOU | 641 | CA | ASN A | 478 | 2546 | 2738 | 2802 | −99 | 60 | 52 | C |

| | | | | | | | | | | |
|---|---|---|---|---|---|---|---|---|---|---|
| ATOM | 642 | C | ASN A | 478 | 6.265 | 26.103 | 69.506 | 1.00 | 21.57 | C |

| | | | | | | | | | | | |
|---|---|---|---|---|---|---|---|---|---|---|---|
| ANISOU | 642 | C | ASN A | 478 | 2603 | 2859 | 2735 | 21 | 54 | 203 | C |

| | | | | | | | | | | |
|---|---|---|---|---|---|---|---|---|---|---|
| ATOM | 643 | O | ASN A | 478 | 5.069 | 26.322 | 69.220 | 1.00 | 22.41 | O |

| | | | | | | | | | | | |
|---|---|---|---|---|---|---|---|---|---|---|---|
| ANISOU | 643 | O | ASN A | 478 | 2725 | 2775 | 3016 | 70 | −23 | 82 | O |

| | | | | | | | | | | |
|---|---|---|---|---|---|---|---|---|---|---|
| ATOM | 644 | CB | ASN A | 478 | 7.751 | 25.461 | 67.584 | 1.00 | 21.76 | C |

| | | | | | | | | | | | |
|---|---|---|---|---|---|---|---|---|---|---|---|
| ANISOU | 644 | CB | ASN A | 478 | 2654 | 2883 | 2733 | 19 | 3 | 49 | C |

| | | | | | | | | | | |
|---|---|---|---|---|---|---|---|---|---|---|
| ATOM | 645 | CG | ASN A | 478 | 8.386 | 25.923 | 66.288 | 1.00 | 22.65 | C |

| | | | | | | | | | | | |
|---|---|---|---|---|---|---|---|---|---|---|---|
| ANISOU | 645 | CG | ASN A | 478 | 2684 | 3068 | 2853 | 11 | 3 | 66 | C |

| | | | | | | | | | | |
|---|---|---|---|---|---|---|---|---|---|---|
| ATOM | 646 | OD1 | ASN A | 478 | 8.519 | 27.114 | 65.995 | 1.00 | 24.82 | O |

| | | | | | | | | | | | |
|---|---|---|---|---|---|---|---|---|---|---|---|
| ANISOU | 646 | OD1 | ASN A | 478 | 3036 | 3296 | 3099 | −33 | 62 | 249 | O |

| | | | | | | | | | | |
|---|---|---|---|---|---|---|---|---|---|---|
| ATOM | 647 | ND2 | ASN A | 478 | 8.783 | 24.946 | 65.498 | 1.00 | 23.76 | N |

| | | | | | | | | | | | |
|---|---|---|---|---|---|---|---|---|---|---|---|
| ANISOU | 647 | ND2 | ASN A | 478 | 2919 | 3399 | 2710 | 60 | 59 | 30 | N |

| | | | | | | | | | | |
|---|---|---|---|---|---|---|---|---|---|---|
| ATOM | 648 | N | LEU A | 479 | 6.619 | 25.353 | 70.543 | 1.00 | 20.09 | N |

| | | | | | | | | | | | |
|---|---|---|---|---|---|---|---|---|---|---|---|
| ANISOU | 648 | N | LEU A | 479 | 2466 | 2627 | 2539 | −44 | 36 | 78 | N |

| | | | | | | | | | | |
|---|---|---|---|---|---|---|---|---|---|---|
| ATOM | 649 | CA | LEU A | 479 | 5.615 | 24.824 | 71.459 | 1.00 | 20.20 | C |

| | | | | | | | | | | | |
|---|---|---|---|---|---|---|---|---|---|---|---|
| ANISOU | 649 | CA | LEU A | 479 | 2473 | 2750 | 2451 | 72 | −4 | 139 | C |

| | | | | | | | | | | |
|---|---|---|---|---|---|---|---|---|---|---|
| ATOM | 650 | C | LEU A | 479 | 4.813 | 25.922 | 72.142 | 1.00 | 21.07 | C |

| | | | | | | | | | | | |
|---|---|---|---|---|---|---|---|---|---|---|---|
| ANISOU | 650 | C | LEU A | 479 | 2598 | 2650 | 2758 | −20 | 96 | 119 | C |

| | | | | | | | | | | |
|---|---|---|---|---|---|---|---|---|---|---|
| ATOM | 651 | O | LEU A | 479 | 3.589 | 25.832 | 72.255 | 1.00 | 21.87 | O |

| | | | | | | | | | | | |
|---|---|---|---|---|---|---|---|---|---|---|---|
| ANISOU | 651 | O | LEU A | 479 | 2428 | 2969 | 2910 | 129 | −127 | 72 | O |

| | | | | | | | | | | |
|---|---|---|---|---|---|---|---|---|---|---|
| ATOM | 652 | CB | LEU A | 479 | 6.325 | 23.969 | 72.539 | 1.00 | 19.68 | C |

| | | | | | | | | | | | |
|---|---|---|---|---|---|---|---|---|---|---|---|
| ANISOU | 652 | CB | LEU A | 479 | 2477 | 2585 | 2416 | 106 | 133 | 195 | C |

| | | | | | | | | | | |
|---|---|---|---|---|---|---|---|---|---|---|
| ATOM | 653 | CG | LEU A | 479 | 5.387 | 23.460 | 73.656 | 1.00 | 20.12 | C |

| | | | | | | | | | | | |
|---|---|---|---|---|---|---|---|---|---|---|---|
| ANISOU | 653 | CG | LEU A | 479 | 2334 | 2667 | 2645 | −205 | −7 | 183 | C |

| | | | | | | | | | | |
|---|---|---|---|---|---|---|---|---|---|---|
| ATOM | 654 | CD1 | LEU A | 479 | 4.357 | 22.478 | 73.093 | 1.00 | 21.56 | C |

| | | | | | | | | | | | |
|---|---|---|---|---|---|---|---|---|---|---|---|
| ANISOU | 654 | CD1 | LEU A | 479 | 2457 | 2926 | 2810 | −162 | −217 | 14 | C |

| | | | | | | | | | | |
|---|---|---|---|---|---|---|---|---|---|---|
| ATOM | 655 | CD2 | LEU A | 479 | 6.287 | 22.769 | 74.684 | 1.00 | 20.67 | C |

| | | | | | | | | | | | |
|---|---|---|---|---|---|---|---|---|---|---|---|
| ANISOU | 655 | CD2 | LEU A | 479 | 2472 | 2719 | 2662 | −62 | 46 | 250 | C |

| | | | | | | | | | | |
|---|---|---|---|---|---|---|---|---|---|---|
| ATOM | 656 | N | SER A | 480 | 5.513 | 26.983 | 72.522 | 1.00 | 20.65 | N |

| | | | | | | | | | | | |
|---|---|---|---|---|---|---|---|---|---|---|---|
| ANISOU | 656 | N | SER A | 480 | 2463 | 2809 | 2576 | −4 | −126 | 19 | N |

| | | | | | | | | | | |
|---|---|---|---|---|---|---|---|---|---|---|
| ATOM | 657 | CA | SER A | 480 | 4.873 | 28.104 | 73.219 | 1.00 | 22.40 | C |

| | | | | | | | | | | | |
|---|---|---|---|---|---|---|---|---|---|---|---|
| ANISOU | 657 | CA | SER A | 480 | 2802 | 2749 | 2961 | 35 | 56 | 34 | C |

| | | | | | | | | | | |
|---|---|---|---|---|---|---|---|---|---|---|
| ATOM | 658 | C | SER A | 480 | 4.069 | 28.969 | 72.258 | 1.00 | 23.42 | C |

| | | | | | | | | | | | |
|---|---|---|---|---|---|---|---|---|---|---|---|
| ANISOU | 658 | C | SER A | 480 | 2807 | 3087 | 3004 | 0 | −17 | 116 | C |

| | | | | | | | | | | |
|---|---|---|---|---|---|---|---|---|---|---|
| ATOM | 659 | O | SER A | 480 | 2.939 | 29.355 | 72.617 | 1.00 | 25.81 | O |

| | | | | | | | | | | | |
|---|---|---|---|---|---|---|---|---|---|---|---|
| ANISOU | 659 | O | SER A | 480 | 3132 | 3547 | 3126 | 279 | 16 | 234 | O |

| | | | | | | | | | | |
|---|---|---|---|---|---|---|---|---|---|---|
| ATOM | 660 | CB | SER A | 480 | 5.912 | 28.907 | 74.021 | 1.00 | 23.15 | C |

| | | | | | | | | | | | |
|---|---|---|---|---|---|---|---|---|---|---|---|
| ANISOU | 660 | CB | SER A | 480 | 2992 | 2555 | 3251 | 111 | 26 | −99 | C |

| | | | | | | | | | | |
|---|---|---|---|---|---|---|---|---|---|---|
| ATOM | 661 | OG | SER A | 480 | 6.868 | 29.472 | 73.141 | 1.00 | 24.84 | O |

| | | | | | | | | | | | |
|---|---|---|---|---|---|---|---|---|---|---|---|
| ANISOU | 661 | OG | SER A | 480 | 3105 | 3002 | 3330 | 75 | 112 | −142 | O |

| | | | | | | | | | | |
|---|---|---|---|---|---|---|---|---|---|---|
| ATOM | 662 | N | GLU A | 481 | 4.617 | 29.234 | 71.070 | 1.00 | 23.26 | N |

| | | | | | | | | | | | |
|---|---|---|---|---|---|---|---|---|---|---|---|
| ANISOU | 662 | N | GLU A | 481 | 2875 | 3087 | 2874 | −89 | −131 | 185 | N |

| | | | | | | | | | | |
|---|---|---|---|---|---|---|---|---|---|---|
| ATOM | 663 | CA | GLU A | 481 | 3.881 | 30.114 | 70.154 | 1.00 | 24.74 | C |

| | | | | | | | | | | | |
|---|---|---|---|---|---|---|---|---|---|---|---|
| ANISOU | 663 | CA | GLU A | 481 | 2990 | 3246 | 3165 | 96 | −95 | 152 | C |

| | | | | | | | | | | |
|---|---|---|---|---|---|---|---|---|---|---|
| ATOM | 664 | C | GLU A | 481 | 2.822 | 29.390 | 69.349 | 1.00 | 23.37 | C |

| | | | | | | | | | | | |
|---|---|---|---|---|---|---|---|---|---|---|---|
| ANISOU | 664 | C | GLU A | 481 | 2884 | 3117 | 2880 | 101 | 95 | 142 | C |

| | | | | | | | | | | |
|---|---|---|---|---|---|---|---|---|---|---|
| ATOM | 665 | O | GLU A | 481 | 1.770 | 29.998 | 69.066 | 1.00 | 24.54 | O |

| | | | | | | | | | | | |
|---|---|---|---|---|---|---|---|---|---|---|---|
| ANISOU | 665 | O | GLU A | 481 | 2813 | 3507 | 3006 | 192 | 34 | 342 | O |

| | | | | | | | | | | |
|---|---|---|---|---|---|---|---|---|---|---|
| ATOM | 666 | CB | GLU A | 481 | 4.922 | 30.825 | 69.221 | 1.00 | 26.78 | C |

| | | | | | | | | | | | |
|---|---|---|---|---|---|---|---|---|---|---|---|
| ANISOU | 666 | CB | GLU A | 481 | 3320 | 3527 | 3330 | 177 | 138 | 185 | C |

| | | | | | | | | | | |
|---|---|---|---|---|---|---|---|---|---|---|
| ATOM | 667 | CG | GLU A | 481 | 5.826 | 31.730 | 70.060 | 1.00 | 31.33 | C |

| | | | | | | | | | | | |
|---|---|---|---|---|---|---|---|---|---|---|---|
| ANISOU | 667 | CG | GLU A | 481 | 3841 | 4016 | 4048 | 18 | −36 | −121 | C |

| | | | | | | | | | | |
|---|---|---|---|---|---|---|---|---|---|---|
| ATOM | 668 | CD | GLU A | 481 | 6.870 | 32.524 | 69.310 | 1.00 | 35.02 | C |

| | | | | | | | | | | | |
|---|---|---|---|---|---|---|---|---|---|---|---|
| ANISOU | 668 | CD | GLU A | 481 | 4406 | 4453 | 4447 | −52 | 162 | 85 | C |

| | | | | | | | | | | |
|---|---|---|---|---|---|---|---|---|---|---|
| ATOM | 669 | OE1 | GLU A | 481 | 6.924 | 32.439 | 68.073 | 1.00 | 36.89 | O |

| | | | | | | | | | | | |
|---|---|---|---|---|---|---|---|---|---|---|---|
| ANISOU | 669 | OE1 | GLU A | 481 | 4700 | 4849 | 4465 | −56 | 100 | 104 | O |

| | | | | | | | | | | |
|---|---|---|---|---|---|---|---|---|---|---|
| ATOM | 670 | OE2 | GLU A | 481 | 7.689 | 33.257 | 69.925 | 1.00 | 36.88 | O |

| | | | | | | | | | | | |
|---|---|---|---|---|---|---|---|---|---|---|---|
| ANISOU | 670 | OE2 | GLU A | 481 | 4515 | 4544 | 4954 | −156 | 167 | 25 | O |

| | | | | | | | | | | |
|---|---|---|---|---|---|---|---|---|---|---|
| ATOM | 671 | N | ARG A | 482 | 3.015 | 28.135 | 68.992 | 1.00 | 21.49 | N |

| | | | | | | | | | | | |
|---|---|---|---|---|---|---|---|---|---|---|---|
| ANISOU | 671 | N | ARG A | 482 | 2570 | 3050 | 2543 | 209 | 162 | 258 | N |

| | | | | | | | | | | |
|---|---|---|---|---|---|---|---|---|---|---|
| ATOM | 672 | CA | ARG A | 482 | 2.188 | 27.393 | 68.067 | 1.00 | 22.47 | C |

| | | | | | | | | | | | |
|---|---|---|---|---|---|---|---|---|---|---|---|
| ANISOU | 672 | CA | ARG A | 482 | 2691 | 3057 | 2789 | 155 | 134 | 176 | C |

| | | | | | | | | | | |
|---|---|---|---|---|---|---|---|---|---|---|
| ATOM | 673 | C | ARG A | 482 | 1.696 | 26.055 | 68.572 | 1.00 | 21.74 | C |

| | | | | | | | | | | | |
|---|---|---|---|---|---|---|---|---|---|---|---|
| ANISOU | 673 | C | ARG A | 482 | 2602 | 2996 | 2663 | 193 | 83 | 57 | C |

| | | | | | | | | | | |
|---|---|---|---|---|---|---|---|---|---|---|
| ATOM | 674 | O | ARG A | 482 | 1.110 | 25.246 | 67.875 | 1.00 | 21.16 | O |

| | | | | | | | | | | | |
|---|---|---|---|---|---|---|---|---|---|---|---|
| ANISOU | 674 | O | ARG A | 482 | 2603 | 3095 | 2343 | −3 | 172 | 241 | O |

| | | | | | | | | | | |
|---|---|---|---|---|---|---|---|---|---|---|
| ATOM | 675 | CB | ARG A | 482 | 2.993 | 27.229 | 66.743 | 1.00 | 25.91 | C |

| | | | | | | | | | | | |
|---|---|---|---|---|---|---|---|---|---|---|---|
| ANISOU | 675 | CB | ARG A | 482 | 3226 | 3567 | 3053 | 236 | 324 | 86 | C |

| | | | | | | | | | | |
|---|---|---|---|---|---|---|---|---|---|---|
| ATOM | 676 | CG | ARG A | 482 | 3.148 | 28.523 | 65.918 | 1.00 | 27.55 | C |

| | | | | | | | | | | | |
|---|---|---|---|---|---|---|---|---|---|---|---|
| ANISOU | 676 | CG | ARG A | 482 | 3465 | 3622 | 3380 | 184 | 272 | 167 | C |

| | | | | | | | | | | |
|---|---|---|---|---|---|---|---|---|---|---|
| ATOM | 677 | CD | ARG A | 482 | 4.168 | 28.296 | 64.764 | 1.00 | 29.36 | C |

| | | | | | | | | | | | |
|---|---|---|---|---|---|---|---|---|---|---|---|
| ANISOU | 677 | CD | ARG A | 482 | 3759 | 4020 | 3377 | 128 | 295 | 117 | C |

| | | | | | | | | | | |
|---|---|---|---|---|---|---|---|---|---|---|
| ATOM | 678 | NE | ARG A | 482 | 5.495 | 28.540 | 65.280 | 1.00 | 30.60 | N |

| | | | | | | | | | | | |
|---|---|---|---|---|---|---|---|---|---|---|---|
| ANISOU | 678 | NE | ARG A | 482 | 3833 | 4101 | 3692 | 2 | 249 | 69 | N |

| | | | | | | | | | | |
|---|---|---|---|---|---|---|---|---|---|---|
| ATOM | 679 | CZ | ARG A | 482 | 6.105 | 29.624 | 65.694 | 1.00 | 30.26 | C |

| | | | | | | | | | | | |
|---|---|---|---|---|---|---|---|---|---|---|---|
| ANISOU | 679 | CZ | ARG A | 482 | 3752 | 3949 | 3798 | 15 | 210 | 164 | C |

| | | | | | | | | | | |
|---|---|---|---|---|---|---|---|---|---|---|
| ATOM | 680 | NH1 | ARG A | 482 | 5.499 | 30.809 | 65.603 | 1.00 | 30.80 | N |

| | | | | | | | | | | | |
|---|---|---|---|---|---|---|---|---|---|---|---|
| ANISOU | 680 | NH1 | ARG A | 482 | 3762 | 4052 | 3889 | 113 | 218 | 183 | N |

| | | | | | | | | | | |
|---|---|---|---|---|---|---|---|---|---|---|
| ATOM | 681 | NH2 | ARG A | 482 | 7.344 | 29.589 | 66.196 | 1.00 | 29.80 | N |

| | | | | | | | | | | | |
|---|---|---|---|---|---|---|---|---|---|---|---|
| ANISOU | 681 | NH2 | ARG A | 482 | 3809 | 3799 | 3716 | 66 | 177 | 226 | N |

| | | | | | | | | | | |
|---|---|---|---|---|---|---|---|---|---|---|
| ATOM | 682 | N | GLY A | 483 | 1.720 | 25.912 | 69.906 | 1.00 | 20.37 | N |

| | | | | | | | | | | | |
|---|---|---|---|---|---|---|---|---|---|---|---|
| ANISOU | 682 | N | GLY A | 483 | 2485 | 2747 | 2506 | 107 | 142 | 120 | N |

| | | | | | | | | | | |
|---|---|---|---|---|---|---|---|---|---|---|
| ATOM | 683 | CA | GLY A | 483 | 1.152 | 24.726 | 70.561 | 1.00 | 20.78 | C |

| | | | | | | | | | | | |
|---|---|---|---|---|---|---|---|---|---|---|---|
| ANISOU | 683 | CA | GLY A | 483 | 2327 | 2933 | 2634 | −108 | −108 | 173 | C |

| | | | | | | | | | | |
|---|---|---|---|---|---|---|---|---|---|---|
| ATOM | 684 | C | GLY A | 483 | 1.863 | 23.439 | 70.234 | 1.00 | 22.09 | C |

| | | | | | | | | | | | |
|---|---|---|---|---|---|---|---|---|---|---|---|
| ANISOU | 684 | C | GLY A | 483 | 2734 | 2897 | 2761 | −147 | 1 | 14 | C |

| | | | | | | | | | | |
|---|---|---|---|---|---|---|---|---|---|---|
| ATOM | 685 | O | GLY A | 483 | 2.899 | 23.333 | 69.552 | 1.00 | 22.34 | O |

| | | | | | | | | | | | |
|---|---|---|---|---|---|---|---|---|---|---|---|
| ANISOU | 685 | O | GLY A | 483 | 2793 | 2887 | 2807 | −203 | 46 | 297 | O |

| | | | | | | | | | | |
|---|---|---|---|---|---|---|---|---|---|---|
| ATOM | 686 | N | TYR A | 484 | 1.290 | 22.316 | 70.681 | 1.00 | 23.51 | N |

| | | | | | | | | | | | |
|---|---|---|---|---|---|---|---|---|---|---|---|
| ANISOU | 686 | N | TYR A | 484 | 2961 | 3095 | 2877 | −166 | −53 | 258 | N |

| | | | | | | | | | | |
|---|---|---|---|---|---|---|---|---|---|---|
| ATOM | 687 | CA | TYR A | 484 | 1.840 | 20.996 | 70.398 | 1.00 | 24.18 | C |

| | | | | | | | | | | | |
|---|---|---|---|---|---|---|---|---|---|---|---|
| ANISOU | 687 | CA | TYR A | 484 | 2831 | 3134 | 3222 | −149 | −42 | 113 | C |

| | | | | | | | | | | |
|---|---|---|---|---|---|---|---|---|---|---|
| ATOM | 688 | C | TYR A | 484 | 1.908 | 20.750 | 68.917 | 1.00 | 24.85 | C |

| | | | | | | | | | | | |
|---|---|---|---|---|---|---|---|---|---|---|---|
| ANISOU | 688 | C | TYR A | 484 | 2920 | 3212 | 3309 | −225 | ‘22 | 99 | C |

| | | | | | | | | | | |
|---|---|---|---|---|---|---|---|---|---|---|
| ATOM | 689 | O | TYR A | 484 | 2.829 | 20.134 | 68.373 | 1.00 | 25.68 | O |

| | | | | | | | | | | | |
|---|---|---|---|---|---|---|---|---|---|---|---|
| ANISOU | 689 | O | TYR A | 484 | 2912 | 3530 | 3316 | −89 | −136 | 39 | O |

| | | | | | | | | | | |
|---|---|---|---|---|---|---|---|---|---|---|
| ATOM | 690 | CB | TYR A | 484 | 0.969 | 19.896 | 71.077 | 1.00 | 25.45 | C |

| | | | | | | | | | | | |
|---|---|---|---|---|---|---|---|---|---|---|---|
| ANISOU | 690 | CB | TYR A | 484 | 3023 | 3285 | 3364 | −246 | −41 | 232 | C |

| | | | | | | | | | | |
|---|---|---|---|---|---|---|---|---|---|---|
| ATOM | 691 | CG | TYR A | 484 | 1.446 | 18.538 | 70.621 | 1.00 | 27.57 | C |

| | | | | | | | | | | | |
|---|---|---|---|---|---|---|---|---|---|---|---|
| ANISOU | 691 | CG | TYR A | 484 | 3399 | 3536 | 3540 | −99 | 35 | 59 | C |

| | | | | | | | | | | |
|---|---|---|---|---|---|---|---|---|---|---|
| ATOM | 692 | CD1 | TYR A | 484 | 2.689 | 18.055 | 71.027 | 1.00 | 27.46 | C |

| | | | | | | | | | | | |
|---|---|---|---|---|---|---|---|---|---|---|---|
| ANISOU | 692 | CD1 | TYR A | 484 | 3255 | 3569 | 3609 | −96 | −4 | 195 | C |

| | | | | | | | | | | |
|---|---|---|---|---|---|---|---|---|---|---|
| ATOM | 693 | CD2 | TYR A | 484 | 0.699 | 17.734 | 69.781 | 1.00 | 28.19 | C |

| | | | | | | | | | | | |
|---|---|---|---|---|---|---|---|---|---|---|---|
| ANISOU | 693 | CD2 | TYR A | 484 | 3329 | 3635 | 3746 | −150 | −64 | 126 | C |

| | | | | | | | | | | |
|---|---|---|---|---|---|---|---|---|---|---|
| ATOM | 694 | CE1 | TYR A | 484 | 3.178 | 16.839 | 70.607 | 1.00 | 29.04 | C |

| | | | | | | | | | | | |
|---|---|---|---|---|---|---|---|---|---|---|---|
| ANISOU | 694 | CE1 | TYR A | 484 | 3603 | 3627 | 3805 | −125 | 36 | 71 | C |

| | | | | | | | | | | |
|---|---|---|---|---|---|---|---|---|---|---|
| ATOM | 695 | CE2 | TYR A | 484 | 1.152 | 16.509 | 69.354 | 1.00 | 29.86 | C |

| | | | | | | | | | | | |
|---|---|---|---|---|---|---|---|---|---|---|---|
| ANISOU | 695 | CE2 | TYR A | 484 | 3540 | 3767 | 4040 | −99 | −26 | 4 | C |

| | | | | | | | | | | |
|---|---|---|---|---|---|---|---|---|---|---|
| ATOM | 696 | CZ | TYR A | 484 | 2.395 | 16.051 | 69.774 | 1.00 | 30.17 | C |

| | | | | | | | | | | | |
|---|---|---|---|---|---|---|---|---|---|---|---|
| ANISOU | 696 | CZ | TYR A | 484 | 3708 | 3809 | 3945 | −58 | −88 | 31 | C |

| | | | | | | | | | | |
|---|---|---|---|---|---|---|---|---|---|---|
| ATOM | 697 | OH | TYR A | 484 | 2.837 | 14.836 | 69.323 | 1.00 | 30.19 | O |

| | | | | | | | | | | | |
|---|---|---|---|---|---|---|---|---|---|---|---|
| ANISOU | 697 | OH | TYR A | 484 | 3605 | 3654 | 4210 | −211 | 12 | 57 | O |

| | | | | | | | | | | |
|---|---|---|---|---|---|---|---|---|---|---|
| ATOM | 698 | N | GLN A | 485 | 0.949 | 21.296 | 68.161 | 1.00 | 26.04 | N |

| | | | | | | | | | | | |
|---|---|---|---|---|---|---|---|---|---|---|---|
| ANISOU | 698 | N | GLN A | 485 | 3049 | 3313 | 3531 | ‘57 | −98 | 126 | N |

| | | | | | | | | | | |
|---|---|---|---|---|---|---|---|---|---|---|
| ATOM | 699 | CA | GLN A | 485 | 0.959 | 21.163 | 66.714 | 1.00 | 27.88 | C |

| | | | | | | | | | | | |
|---|---|---|---|---|---|---|---|---|---|---|---|
| ANISOU | 699 | CA | GLN A | 485 | 3497 | 3519 | 3576 | −58 | −1 | 64 | C |

| | | | | | | | | | | |
|---|---|---|---|---|---|---|---|---|---|---|
| ATOM | 700 | C | GLN A | 485 | 2.241 | 21.717 | 66.112 | 1.00 | 27.34 | C |

| | | | | | | | | | | | |
|---|---|---|---|---|---|---|---|---|---|---|---|
| ANISOU | 700 | C | GLN A | 485 | 3425 | 3395 | 3566 | −24 | −96 | 96 | C |

| | | | | | | | | | | |
|---|---|---|---|---|---|---|---|---|---|---|
| ATOM | 701 | O | GLN A | 485 | 2.730 | 21.171 | 65.129 | 1.00 | 28.29 | O |

| | | | | | | | | | | | |
|---|---|---|---|---|---|---|---|---|---|---|---|
| ANISOU | 701 | O | GLN A | 485 | 3608 | 3557 | 3584 | −70 | −99 | 7 | O |

| | | | | | | | | | | |
|---|---|---|---|---|---|---|---|---|---|---|
| ATOM | 702 | CB | GLN A | 485 | −0.295 | 21.813 | 66.109 | 1.00 | 30.16 | C |

| | | | | | | | | | | | |
|---|---|---|---|---|---|---|---|---|---|---|---|
| ANISOU | 702 | CB | GLN A | 485 | 3685 | 3946 | 3827 | 126 | −110 | −2 | C |

| | | | | | | | | | | |
|---|---|---|---|---|---|---|---|---|---|---|
| ATOM | 703 | CG | GLN A | 485 | −1.536 | 21.050 | 66.611 | 1.00 | 33.42 | C |

| | | | | | | | | | | | |
|---|---|---|---|---|---|---|---|---|---|---|---|
| ANISOU | 703 | CG | GLN A | 485 | 4027 | 4272 | 4400 | −94 | 36 | 76 | C |

| | | | | | | | | | | |
|---|---|---|---|---|---|---|---|---|---|---|
| ATOM | 704 | CD | GLN A | 485 | −1.490 | 19.589 | 66.179 | 1.00 | 35.76 | C |

| | | | | | | | | | | | |
|---|---|---|---|---|---|---|---|---|---|---|---|
| ANISOU | 704 | CD | GLN A | 485 | 4525 | 4429 | 4632 | −41 | −25 | −53 | C |

| | | | | | | | | | | |
|---|---|---|---|---|---|---|---|---|---|---|
| ATOM | 705 | OE1 | GLN A | 485 | −1.014 | 19.249 | 65.087 | 1.00 | 36.70 | O |

| | | | | | | | | | | | |
|---|---|---|---|---|---|---|---|---|---|---|---|
| ANISOU | 705 | OE1 | GLN A | 485 | 4726 | 4565 | 4653 | 29 | −49 | −114 | O |

| | | | | | | | | | | |
|---|---|---|---|---|---|---|---|---|---|---|
| ATOM | 706 | NE2 | GLN A | 485 | −1.945 | 18.701 | 67.053 | 1.00 | 36.54 | N |

| | | | | | | | | | | | |
|---|---|---|---|---|---|---|---|---|---|---|---|
| ANISOU | 706 | NE2 | GLN A | 485 | 4660 | 4615 | 4608 | −103 | −63 | −1 | N |

| | | | | | | | | | | |
|---|---|---|---|---|---|---|---|---|---|---|
| ATOM | 707 | N | GLY A | 486 | 2.787 | 22.774 | 66.745 | 1.00 | 25.29 | N |

| | | | | | | | | | | | |
|---|---|---|---|---|---|---|---|---|---|---|---|
| ANISOU | 707 | N | GLY A | 486 | 3218 | 3107 | 3286 | 23 | −103 | 226 | N |

| | | | | | | | | | | |
|---|---|---|---|---|---|---|---|---|---|---|
| ATOM | 708 | CA | GLY A | 486 | 4.035 | 23.339 | 66.255 | 1.00 | 25.80 | C |

| | | | | | | | | | | | |
|---|---|---|---|---|---|---|---|---|---|---|---|
| ANISOU | 708 | CA | GLY A | 486 | 3091 | 3256 | 3458 | 29 | −37 | 123 | C |

| | | | | | | | | | | |
|---|---|---|---|---|---|---|---|---|---|---|
| ATOM | 709 | C | GLY A | 486 | 5.263 | 22.462 | 66.449 | 1.00 | 24.56 | C |

| | | | | | | | | | | | |
|---|---|---|---|---|---|---|---|---|---|---|---|
| ANISOU | 709 | C | GLY A | 486 | 2996 | 3126 | 3210 | −61 | −55 | 97 | C |

| | | | | | | | | | | |
|---|---|---|---|---|---|---|---|---|---|---|
| ATOM | 710 | O | GLY A | 486 | 6.311 | 22.842 | 65.915 | 1.00 | 24.75 | O |

| | | | | | | | | | | | |
|---|---|---|---|---|---|---|---|---|---|---|---|
| ANISOU | 710 | O | GLY A | 486 | 2891 | 3203 | 3310 | 12 | 27 | 77 | O |

| | | | | | | | | | | |
|---|---|---|---|---|---|---|---|---|---|---|
| ATOM | 711 | N | VAL A | 487 | 5.225 | 21.402 | 67.254 | 1.00 | 23.02 | N |

| | | | | | | | | | | | |
|---|---|---|---|---|---|---|---|---|---|---|---|
| ANISOU | 711 | N | VAL A | 487 | 2694 | 2949 | 3101 | −149 | −42 | 20 | N |

| | | | | | | | | | | |
|---|---|---|---|---|---|---|---|---|---|---|
| ATOM | 712 | CA | VAL A | 487 | 6.376 | 20.548 | 67.489 | 1.00 | 23.02 | C |

| | | | | | | | | | | | |
|---|---|---|---|---|---|---|---|---|---|---|---|
| ANISOU | 712 | CA | VAL A | 487 | 3036 | 2745 | 2963 | −56 | −38 | −108 | C |

| | | | | | | | | | | |
|---|---|---|---|---|---|---|---|---|---|---|
| ATOM | 713 | C | VAL A | 487 | 5.971 | 19.077 | 67.352 | 1.00 | 24.30 | C |

| | | | | | | | | | | | |
|---|---|---|---|---|---|---|---|---|---|---|---|
| ANISOU | 713 | C | VAL A | 487 | 3106 | 2829 | 3300 | −145 | −11 | 69 | C |

| | | | | | | | | | | |
|---|---|---|---|---|---|---|---|---|---|---|
| ATOM | 714 | O | VAL A | 487 | 6.737 | 18.185 | 67.657 | 1.00 | 22.28 | O |

| | | | | | | | | | | | |
|---|---|---|---|---|---|---|---|---|---|---|---|
| ANISOU | 714 | O | VAL A | 487 | 2931 | 2709 | 2825 | −205 | −61 | −77 | O |

| | | | | | | | | | | |
|---|---|---|---|---|---|---|---|---|---|---|
| ATOM | 715 | CB | VAL A | 487 | 7.036 | 20.741 | 68.858 | 1.00 | 21.86 | C |

| | | | | | | | | | | | |
|---|---|---|---|---|---|---|---|---|---|---|---|
| ANISOU | 715 | CB | VAL A | 487 | 2751 | 2635 | 2919 | −125 | 54 | −17 | C |

| | | | | | | | | | | |
|---|---|---|---|---|---|---|---|---|---|---|
| ATOM | 716 | CG1 | VAL A | 487 | 7.717 | 22.130 | 68.930 | 1.00 | 21.21 | C |

| | | | | | | | | | | | |
|---|---|---|---|---|---|---|---|---|---|---|---|
| ANISOU | 716 | CG1 | VAL A | 487 | 2762 | 2548 | 2751 | −185 | −30 | 6 | C |

| | | | | | | | | | | |
|---|---|---|---|---|---|---|---|---|---|---|
| ATOM | 717 | CG2 | VAL A | 487 | 6.036 | 20.636 | 70.021 | 1.00 | 22.10 | C |

| | | | | | | | | | | | |
|---|---|---|---|---|---|---|---|---|---|---|---|
| ANISOU | 717 | CG2 | VAL A | 487 | 2802 | 2822 | 2772 | −83 | −18 | −18 | C |

| | | | | | | | | | | |
|---|---|---|---|---|---|---|---|---|---|---|
| ATOM | 718 | N | ARG A | 488 | 4.759 | 18.820 | 66.855 | 1.00 | 27.74 | N |

| | | | | | | | | | | | |
|---|---|---|---|---|---|---|---|---|---|---|---|
| ANISOU | 718 | N | ARG A | 488 | 3337 | 3501 | 3703 | −78 | −270 | 9 | N |

| | | | | | | | | | | |
|---|---|---|---|---|---|---|---|---|---|---|
| ATOM | 719 | CA | ARG A | 488 | 4.289 | 17.434 | 66.723 | 1.00 | 29.32 | C |

| | | | | | | | | | | | |
|---|---|---|---|---|---|---|---|---|---|---|---|
| ANISOU | 719 | CA | ARG A | 488 | 3688 | 3536 | 3918 | −122 | −114 | −55 | C |

| | | | | | | | | | | |
|---|---|---|---|---|---|---|---|---|---|---|
| ATOM | 720 | C | ARG A | 488 | 5.226 | 16.489 | 66.009 | 1.00 | 28.52 | C |

| | | | | | | | | | | | |
|---|---|---|---|---|---|---|---|---|---|---|---|
| ANISOU | 720 | C | ARG A | 488 | 3495 | 3571 | 3769 | −161 | −204 | −53 | C |

| | | | | | | | | | | |
|---|---|---|---|---|---|---|---|---|---|---|
| ATOM | 721 | O | ARG A | 488 | 5.365 | 15.329 | 66.459 | 1.00 | 28.02 | O |

| | | | | | | | | | | | |
|---|---|---|---|---|---|---|---|---|---|---|---|
| ANISOU | 721 | O | ARG A | 488 | 3378 | 3467 | 3799 | 87 | −222 | −186 | O |

| | | | | | | | | | | |
|---|---|---|---|---|---|---|---|---|---|---|
| ATOM | 722 | CB | ARG A | 488 | 2.904 | 17.505 | 66.023 | 1.00 | 33.15 | C |

| | | | | | | | | | | | |
|---|---|---|---|---|---|---|---|---|---|---|---|
| ANISOU | 722 | CB | ARG A | 488 | 3626 | 4398 | 4572 | 34 | −98 | 41 | C |

| | | | | | | | | | | |
|---|---|---|---|---|---|---|---|---|---|---|
| ATOM | 723 | CG | ARG A | 488 | 2.537 | 16.271 | 65.232 | 1.00 | 38.52 | C |

| | | | | | | | | | | | |
|---|---|---|---|---|---|---|---|---|---|---|---|
| ANISOU | 723 | CG | ARG A | 488 | 4835 | 4736 | 5064 | −70 | −20 | −232 | C |

| | | | | | | | | | | |
|---|---|---|---|---|---|---|---|---|---|---|
| ATOM | 724 | CD | ARG A | 488 | 1.369 | 16.524 | 64.248 | 1.00 | 42.82 | C |

| | | | | | | | | | | | |
|---|---|---|---|---|---|---|---|---|---|---|---|
| ANISOU | 724 | CD | ARG A | 488 | 5215 | 5562 | 5493 | −33 | −239 | 46 | C |

| | | | | | | | | | | |
|---|---|---|---|---|---|---|---|---|---|---|
| ATOM | 725 | NE | ARG A | 488 | 1.696 | 15.754 | 63.020 | 1.00 | 46.19 | N |

| | | | | | | | | | | | |
|---|---|---|---|---|---|---|---|---|---|---|---|
| ANISOU | 725 | NE | ARG A | 488 | 5810 | 5958 | 5783 | −63 | −54 | −152 | N |

| | | | | | | | | | | |
|---|---|---|---|---|---|---|---|---|---|---|
| ATOM | 726 | CZ | ARG A | 488 | 1.607 | 16.346 | 61.822 | 1.00 | 48.42 | C |

| | | | | | | | | | | | |
|---|---|---|---|---|---|---|---|---|---|---|---|
| ANISOU | 726 | CZ | ARG A | 488 | 6177 | 6192 | 6027 | −17 | −51 | 53 | C |

| | | | | | | | | | | |
|---|---|---|---|---|---|---|---|---|---|---|
| ATOM | 727 | NH1 | ARG A | 488 | 1.219 | 17.616 | 61.769 | 1.00 | 49.70 | N |

| | | | | | | | | | | | |
|---|---|---|---|---|---|---|---|---|---|---|---|
| ANISOU | 727 | NE1 | ARG A | 488 | 6368 | 6221 | 6292 | 42 | −8 | −18 | N |

| | | | | | | | | | | |
|---|---|---|---|---|---|---|---|---|---|---|
| ATOM | 728 | NH2 | ARG A | 488 | 1.897 | 15.709 | 60.691 | 1.00 | 49.12 | N |

| | | | | | | | | | | | |
|---|---|---|---|---|---|---|---|---|---|---|---|
| ANISOU | 728 | NH2 | ARG A | 488 | 6200 | 6283 | 6181 | −22 | 23 | −48 | N |

| | | | | | | | | | | |
|---|---|---|---|---|---|---|---|---|---|---|
| ATOM | 729 | N | ARG A | 489 | 5.914 | 16.910 | 64.963 | 1.00 | 28.24 | N |

| | | | | | | | | | | | |
|---|---|---|---|---|---|---|---|---|---|---|---|
| ANISOU | 729 | N | ARG A | 489 | 3384 | 3686 | 3658 | −54 | −286 | −113 | N |

| | | | | | | | | | | |
|---|---|---|---|---|---|---|---|---|---|---|
| ATOM | 730 | CA | ARG A | 489 | 6.774 | 16.105 | 64.133 | 1.00 | 29.05 | C |

| | | | | | | | | | | | |
|---|---|---|---|---|---|---|---|---|---|---|---|
| ANISOU | 730 | CA | ARG A | 489 | 3379 | 3834 | 3825 | −80 | −211 | −176 | C |

| | | | | | | | | | | |
|---|---|---|---|---|---|---|---|---|---|---|
| ATOM | 731 | C | ARG A | 489 | 8.214 | 16.032 | 64.629 | 1.00 | 27.47 | C |

| | | | | | | | | | | | |
|---|---|---|---|---|---|---|---|---|---|---|---|
| ANISON | 731 | C | ARG A | 489 | 3253 | 3651 | 3533 | −48 | −94 | −205 | C |

| | | | | | | | | | | |
|---|---|---|---|---|---|---|---|---|---|---|
| ATOM | 732 | O | ARG A | 489 | 9.039 | 15.317 | 64.060 | 1.00 | 26.40 | O |

| | | | | | | | | | | | |
|---|---|---|---|---|---|---|---|---|---|---|---|
| ANISOU | 732 | O | ARG A | 489 | 3222 | 3615 | 3195 | −264 | −251 | −483 | O |

| | | | | | | | | | | |
|---|---|---|---|---|---|---|---|---|---|---|
| ATOM | 733 | CB | ARG A | 489 | 6.757 | 16.707 | 62.710 | 1.00 | 32.28 | C |

| | | | | | | | | | | | |
|---|---|---|---|---|---|---|---|---|---|---|---|
| ANISOU | 733 | CB | ARG A | 489 | 3978 | 4263 | 4024 | −23 | −37 | −26 | C |

| | | | | | | | | | | |
|---|---|---|---|---|---|---|---|---|---|---|
| ATOM | 734 | CG | ARG A | 489 | 5.345 | 16.801 | 62.100 | 1.00 | 35.16 | C |

| | | | | | | | | | | | |
|---|---|---|---|---|---|---|---|---|---|---|---|
| ANISOU | 734 | CG | ARG A | 489 | 4131 | 4656 | 4571 | −37 | −176 | −93 | C |

| | | | | | | | | | | |
|---|---|---|---|---|---|---|---|---|---|---|
| ATOM | 735 | CD | ARG A | 489 | 5.336 | 17.713 | 60.867 | 1.00 | 37.60 | C |

| | | | | | | | | | | | |
|---|---|---|---|---|---|---|---|---|---|---|---|
| ANISOU | 735 | CD | ARG A | 489 | 4691 | 4844 | 4753 | −36 | −179 | 34 | C |

| | | | | | | | | | | |
|---|---|---|---|---|---|---|---|---|---|---|
| ATOM | 736 | NE | ARG A | 489 | 6.298 | 17.234 | 59.890 | 1.00 | 40.46 | N |

| | | | | | | | | | | | |
|---|---|---|---|---|---|---|---|---|---|---|---|
| ANISOU | 736 | NE | ARG A | 489 | 4958 | 5352 | 5064 | −42 | 29 | −13 | N |

| | | | | | | | | | | |
|---|---|---|---|---|---|---|---|---|---|---|
| ATOM | 737 | CZ | ARG A | 489 | 6.094 | 16.403 | 58.876 | 1.00 | 42.42 | C |

| | | | | | | | | | | | |
|---|---|---|---|---|---|---|---|---|---|---|---|
| ANISOU | 737 | CZ | ARG A | 489 | 5348 | 5480 | 5290 | −54 | 9 | −115 | C |

| | | | | | | | | | | |
|---|---|---|---|---|---|---|---|---|---|---|
| ATOM | 738 | NH1 | ARG A | 489 | 7.131 | 16.072 | 58.109 | 1.00 | 42.34 | N |

| | | | | | | | | | | | |
|---|---|---|---|---|---|---|---|---|---|---|---|
| ANISOU | 738 | NH1 | ARG A | 489 | 5279 | 5487 | 5322 | −10 | −25 | −62 | N |

| | | | | | | | | | | |
|---|---|---|---|---|---|---|---|---|---|---|
| ATOM | 739 | NH2 | ARG A | 489 | 4.867 | 15.935 | 58.613 | 1.00 | 43.18 | N |

| | | | | | | | | | | | |
|---|---|---|---|---|---|---|---|---|---|---|---|
| ANISOU | 739 | NH2 | ARG A | 489 | 5386 | 5600 | 5421 | −90 | −54 | −56 | N |

| | | | | | | | | | | |
|---|---|---|---|---|---|---|---|---|---|---|
| ATOM | 740 | N | TRP A | 490 | 8.549 | 16.775 | 65.688 | 1.00 | 25.78 | N |

| | | | | | | | | | | | |
|---|---|---|---|---|---|---|---|---|---|---|---|
| ANISOU | 740 | N | TRP A | 490 | 3103 | 3277 | 3416 | −21 | −30 | −145 | N |

| | | | | | | | | | | |
|---|---|---|---|---|---|---|---|---|---|---|
| ATOM | 741 | CA | TRP A | 490 | 9.951 | 16.828 | 66.092 | 1.00 | 25.12 | C |

| | | | | | | | | | | | |
|---|---|---|---|---|---|---|---|---|---|---|---|
| ANISOU | 741 | CA | TRP A | 490 | 3190 | 3150 | 3204 | −55 | −147 | −154 | C |

| | | | | | | | | | | |
|---|---|---|---|---|---|---|---|---|---|---|
| ATOM | 742 | C | TRP A | 490 | 10.515 | 15.572 | 66.725 | 1.00 | 24.49 | C |

| | | | | | | | | | | | |
|---|---|---|---|---|---|---|---|---|---|---|---|
| ANISOU | 742 | C | TRP A | 490 | 3117 | 3106 | 3082 | −108 | −119 | −127 | C |

| | | | | | | | | | | |
|---|---|---|---|---|---|---|---|---|---|---|
| ATOM | 743 | O | TRP A | 490 | 11.668 | 15.235 | 66.431 | 1.00 | 24.90 | O |

| | | | | | | | | | | | |
|---|---|---|---|---|---|---|---|---|---|---|---|
| ANISOU | 743 | O | TRP A | 490 | 3297 | 3120 | 3043 | −36 | −143 | −88 | O |

| | | | | | | | | | | |
|---|---|---|---|---|---|---|---|---|---|---|
| ATOM | 744 | CB | TRP A | 490 | 10.197 | 18.013 | 67.029 | 1.00 | 24.61 | C |

| | | | | | | | | | | | |
|---|---|---|---|---|---|---|---|---|---|---|---|
| ANISOU | 744 | CB | TRP A | 490 | 3235 | 2872 | 3245 | −49 | −42 | −95 | C |

| | | | | | | | | | | |
|---|---|---|---|---|---|---|---|---|---|---|
| ATOM | 745 | CG | TRP A | 490 | 10.090 | 19.363 | 66.378 | 1.00 | 24.58 | C |

| | | | | | | | | | | | |
|---|---|---|---|---|---|---|---|---|---|---|---|
| ANISOU | 745 | CG | TRP A | 490 | 3171 | 3084 | 3084 | −60 | 17 | 67 | C |

| | | | | | | | | | | |
|---|---|---|---|---|---|---|---|---|---|---|
| ATOM | 746 | CD1 | TRP A | 490 | 9.189 | 19.829 | 65.478 | 1.00 | 24.10 | C |

| | | | | | | | | | | | |
|---|---|---|---|---|---|---|---|---|---|---|---|
| ANISOU | 746 | CD1 | TRP A | 490 | 3049 | 2996 | 3112 | −60 | −5 | −20 | C |

| | | | | | | | | | | |
|---|---|---|---|---|---|---|---|---|---|---|
| ATOM | 747 | CD2 | TRP A | 490 | 11.014 | 20.449 | 66.620 | 1.00 | 24.01 | C |

| | | | | | | | | | | | |
|---|---|---|---|---|---|---|---|---|---|---|---|
| ANISOU | 747 | CD2 | TRP A | 490 | 3192 | 2974 | 2958 | −19 | 29 | −80 | C |

| | | | | | | | | | | |
|---|---|---|---|---|---|---|---|---|---|---|
| ATOM | 748 | NE1 | TRP A | 490 | 9.472 | 21.148 | 65.149 | 1.00 | 24.22 | N |

| | | | | | | | | | | | |
|---|---|---|---|---|---|---|---|---|---|---|---|
| ANISOU | 748 | NE1 | TRP A | 490 | 3193 | 3055 | 2964 | −19 | −54 | 98 | N |

| | | | | | | | | | | |
|---|---|---|---|---|---|---|---|---|---|---|
| ATOM | 749 | CE2 | TRP A | 490 | 10.580 | 21.538 | 65.830 | 1.00 | 24.33 | C |

| | | | | | | | | | | | |
|---|---|---|---|---|---|---|---|---|---|---|---|
| ANISOU | 749 | CE2 | TRP A | 490 | 3162 | 2954 | 3128 | −115 | 3 | −36 | C |

| | | | | | | | | | | |
|---|---|---|---|---|---|---|---|---|---|---|
| ATOM | 750 | CE3 | TRP A | 490 | 12.144 | 20.591 | 67.427 | 1.00 | 24.49 | C |

| | | | | | | | | | | | |
|---|---|---|---|---|---|---|---|---|---|---|---|
| ANISOU | 750 | CE3 | TRP A | 490 | 3075 | 3166 | 3065 | −59 | 49 | −46 | C |

| | | | | | | | | | | |
|---|---|---|---|---|---|---|---|---|---|---|
| ATOM | 751 | CZ2 | TRP A | 490 | 11.238 | 22.778 | 65.836 | 1.00 | 24.13 | C |

| | | | | | | | | | | | |
|---|---|---|---|---|---|---|---|---|---|---|---|
| ANISOU | 751 | CZ2 | TRP A | 490 | 3104 | 2920 | 3145 | −74 | 100 | 37 | C |

| | | | | | | | | | | |
|---|---|---|---|---|---|---|---|---|---|---|
| ATOM | 752 | CZ3 | TRP A | 490 | 12.825 | 21.798 | 67.400 | 1.00 | 23.93 | C |

| | | | | | | | | | | | |
|---|---|---|---|---|---|---|---|---|---|---|---|
| ANISOU | 752 | CZ3 | TRP A | 490 | 3094 | 3066 | 2932 | −35 | 205 | −9 | C |

| | | | | | | | | | | |
|---|---|---|---|---|---|---|---|---|---|---|
| ATOM | 753 | CH2 | TRP A | 490 | 12.358 | 22.856 | 66.605 | 1.00 | 23.70 | C |

| | | | | | | | | | | | |
|---|---|---|---|---|---|---|---|---|---|---|---|
| ANISOU | 753 | CB2 | TRP A | 490 | 3042 | 3054 | 2909 | −55 | 103 | −64 | C |

| | | | | | | | | | | |
|---|---|---|---|---|---|---|---|---|---|---|
| ATOM | 754 | N | MET A | 491 | 9.793 | 14.860 | 67.597 | 1.00 | 24.48 | N |

| | | | | | | | | | | | |
|---|---|---|---|---|---|---|---|---|---|---|---|
| ANISOU | 754 | N | MET A | 491 | 3155 | 3047 | 3097 | −162 | −201 | −129 | N |

| | | | | | | | | | | |
|---|---|---|---|---|---|---|---|---|---|---|
| ATOM | 755 | CA | MET A | 491 | 10.357 | 13.667 | 68.207 | 1.00 | 26.74 | C |

| | | | | | | | | | | | |
|---|---|---|---|---|---|---|---|---|---|---|---|
| ANISOU | 755 | CA | MET A | 491 | 3478 | 3228 | 3454 | −18 | −74 | 0 | C |

| | | | | | | | | | | |
|---|---|---|---|---|---|---|---|---|---|---|
| ATOM | 756 | C | MET A | 491 | 10.653 | 12.586 | 67.163 | 1.00 | 28.07 | C |

| | | | | | | | | | | | |
|---|---|---|---|---|---|---|---|---|---|---|---|
| ANISOU | 756 | C | MET A | 491 | 3601 | 3305 | 3761 | −50 | 33 | −111 | C |

| | | | | | | | | | | |
|---|---|---|---|---|---|---|---|---|---|---|
| ATOM | 757 | O | MET A | 491 | 11.614 | 11.834 | 67.303 | 1.00 | 26.77 | O |

| | | | | | | | | | | | |
|---|---|---|---|---|---|---|---|---|---|---|---|
| ANISOU | 757 | O | MET A | 491 | 3319 | 3037 | 3817 | −348 | −126 | −255 | O |

| | | | | | | | | | | |
|---|---|---|---|---|---|---|---|---|---|---|
| ATOM | 758 | CB | MET A | 491 | 9.429 | 13.124 | 69.294 | 1.00 | 25.41 | C |

| | | | | | | | | | | | |
|---|---|---|---|---|---|---|---|---|---|---|---|
| ANISOU | 758 | CB | MET A | 491 | 3275 | 3010 | 3368 | −49 | −100 | −95 | C |

| | | | | | | | | | | |
|---|---|---|---|---|---|---|---|---|---|---|
| ATOM | 759 | CG | MET A | 491 | 10.043 | 11.980 | 70.104 | 1.00 | 25.95 | C |

| | | | | | | | | | | | |
|---|---|---|---|---|---|---|---|---|---|---|---|
| ANISOU | 759 | CG | MET A | 491 | 3372 | 3092 | 3396 | −27 | −95 | −27 | C |

| | | | | | | | | | | |
|---|---|---|---|---|---|---|---|---|---|---|
| ATOM | 760 | SD | MET A | 491 | 11.455 | 12.453 | 71.124 | 1.00 | 25.36 | S |

| | | | | | | | | | | | |
|---|---|---|---|---|---|---|---|---|---|---|---|
| ANISOU | 760 | SD | MET A | 491 | 3258 | 2972 | 3407 | 59 | −94 | −107 | S |

| | | | | | | | | | | |
|---|---|---|---|---|---|---|---|---|---|---|
| ATOM | 761 | CE | MET A | 491 | 10.687 | 13.294 | 72.510 | 1.00 | 25.75 | C |

| | | | | | | | | | | | |
|---|---|---|---|---|---|---|---|---|---|---|---|
| ANISOU | 761 | CE | MET A | 491 | 3197 | 3275 | 3312 | −36 | 20 | −9 | C |

| | | | | | | | | | | |
|---|---|---|---|---|---|---|---|---|---|---|
| ATOM | 762 | N | LYS A | 492 | 9.839 | 12.508 | 66.105 | 1.00 | 31.38 | N |

| | | | | | | | | | | | |
|---|---|---|---|---|---|---|---|---|---|---|---|
| ANISOU | 762 | N | LYS A | 492 | 3946 | 3914 | 4063 | −27 | −175 | −23 | N |

| | | | | | | | | | | |
|---|---|---|---|---|---|---|---|---|---|---|
| ATOM | 763 | CA | LYS A | 492 | 10.063 | 11.518 | 65.058 | 1.00 | 34.48 | C |

| | | | | | | | | | | | |
|---|---|---|---|---|---|---|---|---|---|---|---|
| ANISOU | 763 | CA | LYS A | 492 | 4505 | 4240 | 4356 | −8 | 16 | −158 | C |

| | | | | | | | | | | |
|---|---|---|---|---|---|---|---|---|---|---|
| ATOM | 764 | C | LYS A | 492 | 11.440 | 11.700 | 64.441 | 1.00 | 34.42 | C |

| | | | | | | | | | | | |
|---|---|---|---|---|---|---|---|---|---|---|---|
| ANISOU | 764 | C | LYS A | 492 | 4472 | 4279 | 4326 | 43 | −13 | −61 | C |

| | | | | | | | | | | |
|---|---|---|---|---|---|---|---|---|---|---|
| ATOM | 765 | O | LYS A | 492 | 12.174 | 10.747 | 64.191 | 1.00 | 33.93 | O |

| | | | | | | | | | | | |
|---|---|---|---|---|---|---|---|---|---|---|---|
| ANISOU | 765 | O | LYS A | 492 | 4582 | 4097 | 4213 | 37 | −71 | −133 | O |

| | | | | | | | | | | |
|---|---|---|---|---|---|---|---|---|---|---|
| ATOM | 766 | CB | LYS A | 492 | 9.001 | 11.616 | 63.954 | 1.00 | 37.40 | C |

| | | | | | | | | | | | |
|---|---|---|---|---|---|---|---|---|---|---|---|
| ANISOU | 766 | CB | LYS A | 492 | 4704 | 4841 | 4665 | −25 | −125 | 62 | C |

| | | | | | | | | | | |
|---|---|---|---|---|---|---|---|---|---|---|
| ATOM | 767 | CG | LYS A | 492 | 8.969 | 10.333 | 63.113 | 1.00 | 41.21 | C |

| | | | | | | | | | | | |
|---|---|---|---|---|---|---|---|---|---|---|---|
| ANISOU | 767 | CG | LYS A | 492 | 5382 | 5069 | 5205 | 28 | 37 | −138 | C |

| | | | | | | | | | | |
|---|---|---|---|---|---|---|---|---|---|---|
| ATOM | 768 | CD | LYS A | 492 | 7.731 | 10.344 | 62.218 | 1.00 | 44.18 | C |

| | | | | | | | | | | | |
|---|---|---|---|---|---|---|---|---|---|---|---|
| ANISOU | 768 | CD | LYS A | 492 | 5511 | 5680 | 5594 | 19 | −123 | −13 | C |

| | | | | | | | | | | |
|---|---|---|---|---|---|---|---|---|---|---|
| ATOM | 769 | CE | LYS A | 492 | 7.872 | 9.308 | 61.102 | 1.00 | 46.40 | C |

| | | | | | | | | | | | |
|---|---|---|---|---|---|---|---|---|---|---|---|
| ANISOU | 769 | CE | LYS A | 492 | 5949 | 5896 | 5786 | 9 | −31 | −123 | C |

| | | | | | | | | | | |
|---|---|---|---|---|---|---|---|---|---|---|
| ATOM | 770 | NZ | LYS A | 492 | 6.977 | 9.699 | 59.965 | 1.00 | 47.84 | N |

| | | | | | | | | | | | |
|---|---|---|---|---|---|---|---|---|---|---|---|
| ANISOU | 770 | NZ | LYS A | 492 | 6110 | 6125 | 5940 | 59 | −106 | 1 | N |

| | | | | | | | | | | |
|---|---|---|---|---|---|---|---|---|---|---|
| ATOM | 771 | N | ARG A | 493 | 11.844 | 12.964 | 64.234 | 1.00 | 33.46 | N |

| | | | | | | | | | | | |
|---|---|---|---|---|---|---|---|---|---|---|---|
| ANISOU | 771 | N | ARG A | 493 | 4409 | 4157 | 4149 | 45 | −35 | −176 | N |

| | | | | | | | | | | |
|---|---|---|---|---|---|---|---|---|---|---|
| ATOM | 772 | CA | ARG A | 493 | 13.157 | 13.256 | 63.687 | 1.00 | 35.01 | C |

| | | | | | | | | | | | |
|---|---|---|---|---|---|---|---|---|---|---|---|
| ANISOU | 772 | CA | ARG A | 493 | 4529 | 4437 | 4336 | −78 | 11 | −97 | C |

| | | | | | | | | | | |
|---|---|---|---|---|---|---|---|---|---|---|
| ATOM | 773 | C | ARG A | 493 | 14.297 | 12.989 | 64.649 | 1.00 | 33.12 | C |

| | | | | | | | | | | | |
|---|---|---|---|---|---|---|---|---|---|---|---|
| ANISOU | 773 | C | ARG A | 493 | 4315 | 3993 | 4275 | −59 | 94 | −97 | C |

| | | | | | | | | | | |
|---|---|---|---|---|---|---|---|---|---|---|
| ATOM | 774 | O | ARG A | 493 | 15.469 | 13.097 | 64.253 | 1.00 | 34.76 | O |

| | | | | | | | | | | | |
|---|---|---|---|---|---|---|---|---|---|---|---|
| ANISOU | 774 | O | ARG A | 493 | 4464 | 4369 | 4376 | −111 | 183 | −161 | O |

| | | | | | | | | | | |
|---|---|---|---|---|---|---|---|---|---|---|
| ATOM | 775 | CB | ARG A | 493 | 13.252 | 14.692 | 63.177 | 1.00 | 37.40 | C |

| | | | | | | | | | | | |
|---|---|---|---|---|---|---|---|---|---|---|---|
| ANISOU | 775 | CB | ARG A | 493 | 4860 | 4461 | 4889 | −10 | 91 | −48 | C |

| | | | | | | | | | | |
|---|---|---|---|---|---|---|---|---|---|---|
| ATOM | 776 | CG | ARG A | 493 | 12.733 | 15.068 | 61.831 | 1.00 | 40.11 | C |

| | | | | | | | | | | | |
|---|---|---|---|---|---|---|---|---|---|---|---|
| ANISOU | 776 | CG | ARG A | 493 | 5084 | 5101 | 5054 | −4 | −2 | 82 | C |

| | | | | | | | | | | |
|---|---|---|---|---|---|---|---|---|---|---|
| ATOM | 777 | CD | ARG A | 493 | 12.285 | 13.921 | 60.962 | 1.00 | 42.49 | C |

| | | | | | | | | | | | |
|---|---|---|---|---|---|---|---|---|---|---|---|
| ANISOU | 777 | CD | ARG A | 493 | 5606 | 5219 | 5321 | −78 | 48 | −68 | C |

| | | | | | | | | | | |
|---|---|---|---|---|---|---|---|---|---|---|
| ATOM | 778 | NE | ARG A | 493 | 11.374 | 14.308 | 59.879 | 1.00 | 44.09 | N |

| | | | | | | | | | | | |
|---|---|---|---|---|---|---|---|---|---|---|---|
| ANISOU | 778 | NE | ARG A | 493 | 5637 | 5487 | 5629 | −60 | −33 | 75 | N |

| | | | | | | | | | | |
|---|---|---|---|---|---|---|---|---|---|---|
| ATOM | 779 | CZ | ARG A | 493 | 11.174 | 13.487 | 58.840 | 1.00 | 45.16 | C |

| | | | | | | | | | | | |
|---|---|---|---|---|---|---|---|---|---|---|---|
| ANISOU | 779 | CZ | ARG A | 493 | 5754 | 5683 | 5720 | −66 | 14 | −36 | C |

| | | | | | | | | | | |
|---|---|---|---|---|---|---|---|---|---|---|
| ATOM | 780 | NH1 | ARG A | 493 | 11.765 | 12.301 | 58.803 | 1.00 | 45.69 | N |

| | | | | | | | | | | | |
|---|---|---|---|---|---|---|---|---|---|---|---|
| ANISOU | 780 | NH1 | ARG A | 493 | 5810 | 5731 | 5820 | −30 | 27 | 0 | N |

| | | | | | | | | | | |
|---|---|---|---|---|---|---|---|---|---|---|
| ATOM | 781 | NH2 | ARG A | 493 | 10.375 | 13.850 | 57.855 | 1.00 | 45.80 | N |

| | | | | | | | | | | | |
|---|---|---|---|---|---|---|---|---|---|---|---|
| ANISOU | 781 | NH2 | ARG A | 493 | 5841 | 5794 | 5767 | −28 | 1 | −15 | N |

| | | | | | | | | | | |
|---|---|---|---|---|---|---|---|---|---|---|
| ATOM | 782 | N | LYS A | 494 | 14.037 | 12.627 | 65.898 | 1.00 | 30.68 | N |

| | | | | | | | | | | | |
|---|---|---|---|---|---|---|---|---|---|---|---|
| ANISOU | 782 | N | LYS A | 494 | 3877 | 3671 | 4110 | −60 | −11 | −215 | N |

| | | | | | | | | | | |
|---|---|---|---|---|---|---|---|---|---|---|
| ATOM | 783 | CA | LYS A | 494 | 15.024 | 12.243 | 66.879 | 1.00 | 31.03 | C |

| | | | | | | | | | | | |
|---|---|---|---|---|---|---|---|---|---|---|---|
| ANISOU | 783 | CA | LYS A | 494 | 3982 | 3858 | 3950 | 6 | 30 | −127 | C |

| | | | | | | | | | | |
|---|---|---|---|---|---|---|---|---|---|---|
| ATOM | 784 | C | LYS A | 494 | 15.262 | 10.725 | 66.784 | 1.00 | 33.20 | C |

| | | | | | | | | | | | |
|---|---|---|---|---|---|---|---|---|---|---|---|
| ANISOU | 784 | C | LYS A | 494 | 4309 | 3944 | 4364 | 45 | 11 | −52 | C |

| | | | | | | | | | | |
|---|---|---|---|---|---|---|---|---|---|---|
| ATOM | 785 | O | LYS A | 494 | 16.042 | 10.141 | 67.536 | 1.00 | 34.30 | O |

| | | | | | | | | | | | |
|---|---|---|---|---|---|---|---|---|---|---|---|
| ANISOU | 785 | O | LYS A | 494 | 4370 | 4226 | 4438 | 83 | −76 | −52 | O |

| | | | | | | | | | | |
|---|---|---|---|---|---|---|---|---|---|---|
| ATOM | 786 | CB | LYS A | 494 | 14.615 | 12.566 | 68.320 | 1.00 | 28.35 | C |

| | | | | | | | | | | | |
|---|---|---|---|---|---|---|---|---|---|---|---|
| ANISOU | 786 | CB | LYS A | 494 | 3587 | 3381 | 3802 | −8 | −111 | −12 | C |

| | | | | | | | | | | |
|---|---|---|---|---|---|---|---|---|---|---|
| ATOM | 787 | CG | LYS A | 494 | 14.719 | 14.058 | 68.625 | 1.00 | 26.48 | C |

| | | | | | | | | | | | |
|---|---|---|---|---|---|---|---|---|---|---|---|
| ANISOU | 787 | CG | LYS A | 494 | 3213 | 3379 | 3469 | −70 | −68 | −85 | C |

| | | | | | | | | | | |
|---|---|---|---|---|---|---|---|---|---|---|
| ATOM | 788 | CD | LYS A | 494 | 16.175 | 14.538 | 68.707 | 1.00 | 24.91 | C |

| | | | | | | | | | | | |
|---|---|---|---|---|---|---|---|---|---|---|---|
| ANISOU | 788 | CD | LYS A | 494 | 3102 | 3178 | 3184 | 16 | −77 | −134 | C |

| | | | | | | | | | | |
|---|---|---|---|---|---|---|---|---|---|---|
| ATOM | 789 | CE | LYS A | 494 | 16.185 | 16.052 | 68.896 | 1.00 | 25.48 | C |

| | | | | | | | | | | | |
|---|---|---|---|---|---|---|---|---|---|---|---|
| ANISOU | 789 | CE | LYS A | 494 | 3000 | 3264 | 3417 | −109 | −54 | −60 | C |

| | | | | | | | | | | |
|---|---|---|---|---|---|---|---|---|---|---|
| ATOM | 790 | NZ | LYS A | 494 | 17.581 | 16.541 | 69.066 | 1.00 | 24.30 | N |

| | | | | | | | | | | | |
|---|---|---|---|---|---|---|---|---|---|---|---|
| ANISOU | 790 | NZ | LYS A | 494 | 2909 | 2932 | 3393 | −81 | 41 | −214 | N |

| | | | | | | | | | | |
|---|---|---|---|---|---|---|---|---|---|---|
| ATOM | 791 | N | LYS A | 495 | 14.470 | 10.041 | 65.939 | 1.00 | 34.71 | N |

| | | | | | | | | | | | |
|---|---|---|---|---|---|---|---|---|---|---|---|
| ANISOU | 791 | N | LYS A | 495 | 4460 | 4331 | 4395 | 17 | −41 | −126 | N |

| | | | | | | | | | | |
|---|---|---|---|---|---|---|---|---|---|---|
| ATOM | 792 | CA | LYS A | 495 | 14.621 | 8.600 | 65.772 | 1.00 | 35.25 | C |

| | | | | | | | | | | | |
|---|---|---|---|---|---|---|---|---|---|---|---|
| ANISOU | 792 | CA | LYS A | 495 | 4594 | 4283 | 4518 | −129 | −65 | −37 | C |

| | | | | | | | | | | |
|---|---|---|---|---|---|---|---|---|---|---|
| ATOM | 793 | C | LYS A | 495 | 14.318 | 7.849 | 67.065 | 1.00 | 33.30 | C |

| | | | | | | | | | | | |
|---|---|---|---|---|---|---|---|---|---|---|---|
| ANISOU | 793 | C | LYS A | 495 | 4210 | 4099 | 4343 | −37 | −45 | −203 | C |

| | | | | | | | | | | |
|---|---|---|---|---|---|---|---|---|---|---|
| ATOM | 794 | O | LYS A | 495 | 14.968 | 6.844 | 67.371 | 1.00 | 32.25 | O |

| | | | | | | | | | | | |
|---|---|---|---|---|---|---|---|---|---|---|---|
| ANISOU | 794 | O | LYS A | 495 | 3975 | 3959 | 4320 | −150 | −63 | −214 | O |

| | | | | | | | | | | |
|---|---|---|---|---|---|---|---|---|---|---|
| ATOM | 795 | CB | LYS A | 495 | 16.011 | 8.147 | 65.344 | 1.00 | 38.76 | C |

| | | | | | | | | | | | |
|---|---|---|---|---|---|---|---|---|---|---|---|
| ANISOU | 795 | CB | LYS A | 495 | 4839 | 4867 | 5021 | 89 | 0 | −105 | C |

| | | | | | | | | | | |
|---|---|---|---|---|---|---|---|---|---|---|
| ATOM | 796 | CG | LYS A | 495 | 16.685 | 8.660 | 64.102 | 1.00 | 41.88 | C |

| | | | | | | | | | | | |
|---|---|---|---|---|---|---|---|---|---|---|---|
| ANISOU | 796 | CG | LYS A | 495 | 5401 | 5220 | 5291 | −103 | 142 | 37 | C |

| | | | | | | | | | | |
|---|---|---|---|---|---|---|---|---|---|---|
| ATOM | 797 | CD | LYS A | 495 | 15.714 | 8.934 | 62.972 | 1.00 | 44.34 | C |

| | | | | | | | | | | | |
|---|---|---|---|---|---|---|---|---|---|---|---|
| ANISOU | 797 | CD | LYS A | 485 | 5694 | 5558 | 5595 | 9 | −73 | 54 | C |

| | | | | | | | | | | |
|---|---|---|---|---|---|---|---|---|---|---|
| ATOM | 798 | CE | LYS A | 495 | 16.443 | 8.894 | 61.627 | 1.00 | 46.64 | C |

| | | | | | | | | | | | |
|---|---|---|---|---|---|---|---|---|---|---|---|
| ANISOU | 798 | CE | LYS A | 495 | 5994 | 5952 | 5774 | −17 | 74 | 6 | C |

| | | | | | | | | | | |
|---|---|---|---|---|---|---|---|---|---|---|
| ATOM | 799 | NZ | LYS A | 495 | 15.419 | 8.970 | 60.528 | 1.00 | 48.11 | N |

| | | | | | | | | | | | |
|---|---|---|---|---|---|---|---|---|---|---|---|
| ANISOU | 799 | NZ | LYS A | 495 | 6086 | 6212 | 5983 | 48 | −60 | 58 | N |

| | | | | | | | | | | |
|---|---|---|---|---|---|---|---|---|---|---|
| ATOM | 800 | N | THR A | 496 | 13.305 | 8.310 | 67.794 | 1.00 | 30.86 | N |

| | | | | | | | | | | | |
|---|---|---|---|---|---|---|---|---|---|---|---|
| ANISOU | 800 | N | THR A | 496 | 4027 | 3600 | 4100 | −104 | −105 | −151 | N |

| | | | | | | | | | | |
|---|---|---|---|---|---|---|---|---|---|---|
| ATOM | 801 | CA | THR A | 496 | 12.975 | 7.621 | 69.039 | 1.00 | 30.54 | C |

| | | | | | | | | | | | |
|---|---|---|---|---|---|---|---|---|---|---|---|
| ANISOU | 801 | CA | THR A | 496 | 4015 | 3560 | 4028 | −9 | −32 | −205 | C |

| | | | | | | | | | | |
|---|---|---|---|---|---|---|---|---|---|---|
| ATOM | 802 | C | THR A | 496 | 11.590 | 8.060 | 69.460 | 1.00 | 28.33 | C |

| | | | | | | | | | | | |
|---|---|---|---|---|---|---|---|---|---|---|---|
| ANISOU | 802 | C | THR A | 496 | 3828 | 3160 | 3777 | −207 | −145 | −204 | C |

| | | | | | | | | | | |
|---|---|---|---|---|---|---|---|---|---|---|
| ATOM | 803 | O | THR A | 496 | 10.925 | 8.770 | 68.700 | 1.00 | 28.76 | O |

| | | | | | | | | | | | |
|---|---|---|---|---|---|---|---|---|---|---|---|
| ANISOU | 803 | O | THR A | 496 | 3934 | 3185 | 3808 | −142 | −195 | −243 | O |

| | | | | | | | | | | |
|---|---|---|---|---|---|---|---|---|---|---|
| ATOM | 804 | CB | THR A | 496 | 14.032 | 7.883 | 70.122 | 1.00 | 31.29 | C |

| | | | | | | | | | | | |
|---|---|---|---|---|---|---|---|---|---|---|---|
| ANISOU | 804 | CB | THR A | 496 | 3984 | 3764 | 4143 | 6 | −33 | −179 | C |

| | | | | | | | | | | |
|---|---|---|---|---|---|---|---|---|---|---|
| ATOM | 805 | OG1 | THR A | 496 | 13.807 | 7.021 | 71.240 | 1.00 | 31.57 | O |

| | | | | | | | | | | | |
|---|---|---|---|---|---|---|---|---|---|---|---|
| ANISOU | 805 | OG1 | THR A | 496 | 3995 | 3872 | 4129 | 147 | −24 | −158 | O |

| | | | | | | | | | | |
|---|---|---|---|---|---|---|---|---|---|---|
| ATOM | 806 | CG2 | THR A | 496 | 13.879 | 9.339 | 70.594 | 1.00 | 31.45 | C |

| | | | | | | | | | | | |
|---|---|---|---|---|---|---|---|---|---|---|---|
| ANISOU | 806 | CG2 | THR A | 496 | 4191 | 3755 | 4003 | 42 | 73 | −152 | C |

| | | | | | | | | | | |
|---|---|---|---|---|---|---|---|---|---|---|
| ATOM | 807 | N | GLN A | 497 | 11.071 | 7.488 | 70.530 | 1.00 | 28.71 | N |

| | | | | | | | | | | | |
|---|---|---|---|---|---|---|---|---|---|---|---|
| ANISOU | 807 | N | GLN A | 497 | 3772 | 3324 | 3814 | −97 | −20 | −204 | N |

| | | | | | | | | | | |
|---|---|---|---|---|---|---|---|---|---|---|
| ATOM | 808 | CA | GLN A | 497 | 9.724 | 7.776 | 70.997 | 1.00 | 28.42 | C |

| | | | | | | | | | | | |
|---|---|---|---|---|---|---|---|---|---|---|---|
| ANISOU | 808 | CA | GLN A | 497 | 3655 | 3359 | 3782 | −231 | −49 | −23 | C |

| | | | | | | | | | | |
|---|---|---|---|---|---|---|---|---|---|---|
| ATOM | 809 | C | GLN A | 497 | 9.771 | 8.149 | 72.464 | 1.00 | 26.85 | C |

| | | | | | | | | | | | |
|---|---|---|---|---|---|---|---|---|---|---|---|
| ANISOU | 809 | C | GLN A | 497 | 3409 | 3009 | 3784 | −247 | −26 | −31 | C |

| | | | | | | | | | | |
|---|---|---|---|---|---|---|---|---|---|---|
| ATOM | 810 | O | GLN A | 497 | 10.605 | 7.681 | 73.248 | 1.00 | 27.05 | O |

| | | | | | | | | | | | |
|---|---|---|---|---|---|---|---|---|---|---|---|
| ANISOU | 810 | O | GLN A | 497 | 3493 | 2806 | 3978 | −138 | −91 | −144 | O |

| | | | | | | | | | | |
|---|---|---|---|---|---|---|---|---|---|---|
| ATOM | 811 | CB | GLN A | 497 | 8.806 | 6.534 | 70.829 | 1.00 | 30.38 | C |

| | | | | | | | | | | | |
|---|---|---|---|---|---|---|---|---|---|---|---|
| ANISOU | 811 | CB | GLN A | 497 | 3907 | 3450 | 4184 | −306 | −41 | −80 | C |

| | | | | | | | | | | |
|---|---|---|---|---|---|---|---|---|---|---|
| ATOM | 812 | CG | GLN A | 497 | 8.741 | 6.009 | 69.409 | 1.00 | 32.97 | C |

| | | | | | | | | | | | |
|---|---|---|---|---|---|---|---|---|---|---|---|
| ANISOU | 812 | CG | GLN A | 497 | 4319 | 3985 | 4221 | −210 | −97 | −101 | C |

| | | | | | | | | | | |
|---|---|---|---|---|---|---|---|---|---|---|
| ATOM | 913 | CD | GLN A | 497 | 9.923 | 5.137 | 69.015 | 1.00 | 34.66 | C |

| | | | | | | | | | | | |
|---|---|---|---|---|---|---|---|---|---|---|---|
| ANISOU | 813 | CD | GLN A | 497 | 4538 | 4150 | 4481 | −138 | −45 | −207 | C |

| | | | | | | | | | | |
|---|---|---|---|---|---|---|---|---|---|---|
| ATOM | 814 | OE1 | GLN A | 497 | 10.465 | 4.356 | 69.800 | 1.00 | 36.17 | O |

| | | | | | | | | | | | |
|---|---|---|---|---|---|---|---|---|---|---|---|
| ANISOU | 814 | OE1 | GLN A | 497 | 4886 | 4141 | 4715 | −122 | −163 | 167 | O |

| | | | | | | | | | | |
|---|---|---|---|---|---|---|---|---|---|---|
| ATOM | 815 | NE2 | GLN A | 497 | 10.403 | 5.326 | 67.792 | 1.00 | 35.09 | N |

| | | | | | | | | | | | |
|---|---|---|---|---|---|---|---|---|---|---|---|
| ANISOU | 815 | NE2 | GLN A | 497 | 4753 | 4051 | 4527 | −252 | 47 | −169 | N |

| | | | | | | | | | | |
|---|---|---|---|---|---|---|---|---|---|---|
| ATOM | 816 | N | ILE A | 498 | 8.856 | 8.999 | 72.909 | 1.00 | 26.96 | N |

| | | | | | | | | | | | |
|---|---|---|---|---|---|---|---|---|---|---|---|
| ANISOU | 816 | N | ILE A | 498 | 3376 | 3230 | 3639 | −207 | 42 | 54 | N |

| | | | | | | | | | | |
|---|---|---|---|---|---|---|---|---|---|---|
| ATOM | 817 | CA | ILE A | 498 | 8.834 | 9.464 | 74.294 | 1.00 | 26.10 | C |

| | | | | | | | | | | | |
|---|---|---|---|---|---|---|---|---|---|---|---|
| ANISOU | 817 | CA | ILE A | 498 | 3234 | 3081 | 3604 | −228 | −32 | −44 | C |

| | | | | | | | | | | |
|---|---|---|---|---|---|---|---|---|---|---|
| ATOM | 818 | C | ILE A | 498 | 8.820 | 8.344 | 75.308 | 1.00 | 27.19 | C |

| | | | | | | | | | | | |
|---|---|---|---|---|---|---|---|---|---|---|---|
| ANISOU | 818 | C | ILE A | 498 | 3387 | 3154 | 3788 | −110 | −88 | 42 | C |

| | | | | | | | | | | |
|---|---|---|---|---|---|---|---|---|---|---|
| ATOM | 819 | O | ILE A | 498 | 9.458 | 8.459 | 76.367 | 1.00 | 26.26 | O |

| | | | | | | | | | | | |
|---|---|---|---|---|---|---|---|---|---|---|---|
| ANISOU | 819 | O | ILE A | 498 | 3535 | 2690 | 3762 | −163 | −90 | −80 | O |

| | | | | | | | | | | |
|---|---|---|---|---|---|---|---|---|---|---|
| ATOM | 820 | CB | ILE A | 498 | 7.747 | 10.513 | 74.568 | 1.00 | 25.77 | C |

| | | | | | | | | | | | |
|---|---|---|---|---|---|---|---|---|---|---|---|
| ANISOU | 820 | CB | ILE A | 498 | 3081 | 3285 | 3425 | −121 | −70 | 123 | C |

| | | | | | | | | | | |
|---|---|---|---|---|---|---|---|---|---|---|
| ATOM | 821 | CG1 | ILE A | 498 | 7.970 | 11.181 | 75.925 | 1.00 | 25.46 | C |

| | | | | | | | | | | | |
|---|---|---|---|---|---|---|---|---|---|---|---|
| ANISOU | 821 | CG1 | ILE A | 498 | 3048 | 3008 | 3616 | −153 | −24 | −8 | C |

| | | | | | | | | | | |
|---|---|---|---|---|---|---|---|---|---|---|
| ATOM | 822 | CG2 | ILE A | 498 | 6.327 | 9.955 | 74.522 | 1.00 | 25.92 | C |

| | | | | | | | | | | | |
|---|---|---|---|---|---|---|---|---|---|---|---|
| ANISOU | 822 | CG2 | ILE A | 498 | 3222 | 3123 | 3502 | −280 | 105 | 59 | C |

| | | | | | | | | | | |
|---|---|---|---|---|---|---|---|---|---|---|
| ATOM | 823 | CD1 | ILE A | 498 | 7.278 | 12.526 | 76.083 | 1.00 | 26.13 | C |

| | | | | | | | | | | | |
|---|---|---|---|---|---|---|---|---|---|---|---|
| ANISOU | 823 | CD1 | ILE A | 498 | 3187 | 3171 | 3572 | 0 | −129 | −26 | C |

| | | | | | | | | | | |
|---|---|---|---|---|---|---|---|---|---|---|
| ATOM | 824 | N | ASP A | 499 | 8.157 | 7.193 | 75.038 | 1.00 | 28.65 | N |

| | | | | | | | | | | | |
|---|---|---|---|---|---|---|---|---|---|---|---|
| ANISOU | 824 | N | ASP A | 499 | 3590 | 3265 | 4028 | −176 | −190 | −141 | N |

| | | | | | | | | | | |
|---|---|---|---|---|---|---|---|---|---|---|
| ATOM | 825 | CA | ASP A | 499 | 8.173 | 6.129 | 76.050 | 1.00 | 30.82 | C |

| | | | | | | | | | | | |
|---|---|---|---|---|---|---|---|---|---|---|---|
| ANISOU | 825 | CA | ASP A | 499 | 3931 | 3711 | 4067 | −163 | −107 | 52 | C |

| | | | | | | | | | | |
|---|---|---|---|---|---|---|---|---|---|---|
| ATOM | 826 | C | ASP A | 499 | 9.494 | 5.403 | 76.222 | 1.00 | 31.39 | C |

| | | | | | | | | | | | |
|---|---|---|---|---|---|---|---|---|---|---|---|
| ANISOU | 826 | C | ASP A | 499 | 3956 | 3756 | 4214 | −187 | −95 | −34 | C |

| | | | | | | | | | | |
|---|---|---|---|---|---|---|---|---|---|---|
| ATOM | 827 | O | ASP A | 499 | 9.627 | 4.594 | 77.172 | 1.00 | 31.89 | O |

| | | | | | | | | | | | |
|---|---|---|---|---|---|---|---|---|---|---|---|
| ANISOU | 827 | O | ASP A | 499 | 4017 | 3673 | 4427 | −142 | −110 | 87 | O |

| | | | | | | | | | | |
|---|---|---|---|---|---|---|---|---|---|---|
| ATOM | 828 | CB | ASP A | 499 | 7.030 | 5.141 | 75.773 | 1.00 | 33.24 | C |

| | | | | | | | | | | | |
|---|---|---|---|---|---|---|---|---|---|---|---|
| ANISOU | 828 | CB | ASP A | 499 | 4243 | 3872 | 4514 | −317 | −109 | 78 | C |

| | | | | | | | | | | |
|---|---|---|---|---|---|---|---|---|---|---|
| ATOM | 829 | CG | ASP A | 499 | 7.192 | 4.219 | 74.608 | 1.00 | 36.15 | C |

| | | | | | | | | | | | |
|---|---|---|---|---|---|---|---|---|---|---|---|
| ANISOU | 829 | CG | ASP A | 499 | 4655 | 4509 | 4572 | −143 | −61 | −89 | C |

| | | | | | | | | | | |
|---|---|---|---|---|---|---|---|---|---|---|
| ATOM | 830 | OD1 | ASP A | 499 | 8.219 | 4.099 | 73.946 | 1.00 | 37.41 | O |

| | | | | | | | | | | | |
|---|---|---|---|---|---|---|---|---|---|---|---|
| ANISOU | 830 | OD1 | ASP A | 499 | 4793 | 4680 | 4742 | −284 | −42 | −127 | O |

| | | | | | | | | | | |
|---|---|---|---|---|---|---|---|---|---|---|
| ATOM | 831 | OD2 | ASP A | 499 | 6.193 | 3.482 | 74.333 | 1.00 | 38.16 | O |

| | | | | | | | | | | | |
|---|---|---|---|---|---|---|---|---|---|---|---|
| ANISOU | 831 | OD2 | ASP A | 499 | 4859 | 4729 | 4912 | −246 | −114 | −63 | O |

| | | | | | | | | | | |
|---|---|---|---|---|---|---|---|---|---|---|
| ATOM | 832 | N | LYS A | 500 | 10.520 | 5.686 | 75.422 | 1.00 | 30.89 | N |

| | | | | | | | | | | | |
|---|---|---|---|---|---|---|---|---|---|---|---|
| ANISOU | 832 | N | LYS A | 500 | 3981 | 3614 | 4141 | −98 | −87 | −98 | N |

| | | | | | | | | | | |
|---|---|---|---|---|---|---|---|---|---|---|
| ATOM | 833 | CA | LYS A | 500 | 11.827 | 5.064 | 75.640 | 1.00 | 30.61 | C |

| | | | | | | | | | | | |
|---|---|---|---|---|---|---|---|---|---|---|---|
| ANISOU | 833 | CA | LYS A | 500 | 3892 | 3519 | 4220 | −198 | −178 | −147 | C |

| | | | | | | | | | | |
|---|---|---|---|---|---|---|---|---|---|---|
| ATOM | 834 | C | LYS A | 500 | 12.819 | 5.982 | 76.349 | 1.00 | 28.91 | C |

| | | | | | | | | | | | |
|---|---|---|---|---|---|---|---|---|---|---|---|
| ANISOU | 834 | C | LYS A | 500 | 3781 | 3245 | 3959 | −28 | −94 | −128 | C |

| | | | | | | | | | | |
|---|---|---|---|---|---|---|---|---|---|---|
| ATOM | 835 | O | LYS A | 500 | 13.970 | 5.604 | 76.514 | 1.00 | 28.04 | O |

| | | | | | | | | | | | |
|---|---|---|---|---|---|---|---|---|---|---|---|
| ANISOU | 835 | O | LYS A | 500 | 3667 | 2835 | 4153 | −183 | −240 | −304 | O |

| | | | | | | | | | | |
|---|---|---|---|---|---|---|---|---|---|---|
| ATOM | 836 | CB | LYS A | 500 | 12.373 | 4.629 | 74.254 | 1.00 | 34.11 | C |

| | | | | | | | | | | | |
|---|---|---|---|---|---|---|---|---|---|---|---|
| ANISOU | 836 | CB | LYS A | 500 | 4305 | 4243 | 4411 | 90 | 48 | −77 | C |

| | | | | | | | | | | |
|---|---|---|---|---|---|---|---|---|---|---|
| ATOM | 837 | CG | LYS A | 500 | 11.456 | 3.400 | 73.943 | 1.00 | 37.41 | C |

| | | | | | | | | | | | |
|---|---|---|---|---|---|---|---|---|---|---|---|
| ANISOU | 837 | CG | LYS A | 500 | 4684 | 4600 | 4931 | −123 | −80 | −94 | C |

| | | | | | | | | | | |
|---|---|---|---|---|---|---|---|---|---|---|
| ATOM | 838 | CD | LYS A | 500 | 11.785 | 2.652 | 72.688 | 1.00 | 39.98 | C |

| | | | | | | | | | | | |
|---|---|---|---|---|---|---|---|---|---|---|---|
| ANISOU | 838 | CD | LYS A | 500 | 5091 | 5063 | 5037 | 54 | −10 | −138 | C |

| | | | | | | | | | | |
|---|---|---|---|---|---|---|---|---|---|---|
| ATOM | 839 | CE | LYS A | 500 | 10.608 | 1.784 | 72.266 | 1.00 | 41.14 | C |

| | | | | | | | | | | | |
|---|---|---|---|---|---|---|---|---|---|---|---|
| ANISOU | 839 | CE | LYS A | 500 | 5202 | 5084 | 5345 | −48 | −37 | −35 | C |

| | | | | | | | | | | |
|---|---|---|---|---|---|---|---|---|---|---|
| ATOM | 840 | NZ | LYS A | 500 | 9.401 | 2.609 | 71.943 | 1.00 | 41.53 | N |

| | | | | | | | | | | | |
|---|---|---|---|---|---|---|---|---|---|---|---|
| ANISOU | 840 | NZ | LYS A | 500 | 5193 | 5156 | 5430 | −56 | −130 | −88 | N |

| | | | | | | | | | | |
|---|---|---|---|---|---|---|---|---|---|---|
| ATOM | 841 | N | LEU A | 501 | 12.366 | 7.190 | 76.683 | 1.00 | 27.35 | N |

| | | | | | | | | | | | |
|---|---|---|---|---|---|---|---|---|---|---|---|
| ANISOU | 841 | N | LEU A | 501 | 3592 | 3084 | 3714 | −198 | −184 | −169 | N |

| | | | | | | | | | | |
|---|---|---|---|---|---|---|---|---|---|---|
| ATOM | 842 | CA | LEU A | 501 | 13.338 | 8.128 | 77.273 | 1.00 | 24.06 | C |

| | | | | | | | | | | | |
|---|---|---|---|---|---|---|---|---|---|---|---|
| ANISOU | 842 | CA | LEU A | 501 | 3309 | 2524 | 3307 | 12 | −104 | 44 | C |

| | | | | | | | | | | |
|---|---|---|---|---|---|---|---|---|---|---|
| ATOM | 843 | C | LEU A | 501 | 13.340 | 8.270 | 78.776 | 1.00 | 24.65 | C |

| | | | | | | | | | | | |
|---|---|---|---|---|---|---|---|---|---|---|---|
| ANISOU | 843 | C | LEU A | 501 | 3295 | 2748 | 3322 | −48 | −154 | −13 | C |

| | | | | | | | | | | |
|---|---|---|---|---|---|---|---|---|---|---|
| ATOM | 844 | O | LEU A | 501 | 12.382 | 8.095 | 79.530 | 1.00 | 24.51 | O |

| | | | | | | | | | | | |
|---|---|---|---|---|---|---|---|---|---|---|---|
| ANISOU | 844 | O | LEU A | 501 | 3369 | 2662 | 3281 | −328 | −100 | −137 | O |

| | | | | | | | | | | |
|---|---|---|---|---|---|---|---|---|---|---|
| ATOM | 845 | CB | LEU A | 501 | 12.991 | 9.507 | 76.626 | 1.00 | 22.80 | C |

| | | | | | | | | | | | |
|---|---|---|---|---|---|---|---|---|---|---|---|
| ANISOU | 845 | CB | LEU A | 501 | 3188 | 2270 | 3207 | −32 | −74 | −72 | C |

| | | | | | | | | | | |
|---|---|---|---|---|---|---|---|---|---|---|
| ATOM | 846 | CG | LEU A | 501 | 13.115 | 9.641 | 75.114 | 1.00 | 23.38 | C |

| | | | | | | | | | | | |
|---|---|---|---|---|---|---|---|---|---|---|---|
| ANISOU | 846 | CG | LEU A | 501 | 3208 | 2415 | 3262 | 16 | −170 | −54 | C |

| | | | | | | | | | | |
|---|---|---|---|---|---|---|---|---|---|---|
| ATOM | 847 | CD1 | LEU A | 501 | 12.638 | 11.028 | 74.620 | 1.00 | 23.49 | C |

| | | | | | | | | | | | |
|---|---|---|---|---|---|---|---|---|---|---|---|
| ANISOU | 847 | CD1 | LEU A | 501 | 3282 | 2413 | 3231 | 24 | −189 | −102 | C |

| | | | | | | | | | | |
|---|---|---|---|---|---|---|---|---|---|---|
| ATOM | 848 | CD2 | LEU A | 501 | 14.542 | 9.352 | 74.648 | 1.00 | 25.32 | C |

| | | | | | | | | | | | |
|---|---|---|---|---|---|---|---|---|---|---|---|
| ANISOU | 848 | CD2 | LEU A | 501 | 3479 | 2848 | 3293 | −21 | 121 | −87 | C |

| | | | | | | | | | | |
|---|---|---|---|---|---|---|---|---|---|---|
| ATOM | 849 | N | ASP A | 502 | 14.522 | 8.677 | 79.293 | 1.00 | 23.43 | N |

| | | | | | | | | | | | |
|---|---|---|---|---|---|---|---|---|---|---|---|
| ANISOU | 849 | N | ASP A | 502 | 3120 | 2349 | 3436 | −144 | −18 | 52 | N |

| | | | | | | | | | | |
|---|---|---|---|---|---|---|---|---|---|---|
| ATOM | 850 | CA | ASP A | 502 | 14.654 | 8.980 | 80.712 | 1.00 | 23.57 | C |

| | | | | | | | | | | | |
|---|---|---|---|---|---|---|---|---|---|---|---|
| ANISOU | 850 | CA | ASP A | 502 | 3257 | 2325 | 3373 | −127 | 100 | 39 | C |

| | | | | | | | | | | |
|---|---|---|---|---|---|---|---|---|---|---|
| ATOM | 851 | C | ASP A | 502 | 14.382 | 10.496 | 80.901 | 1.00 | 22.78 | C |

| | | | | | | | | | | | |
|---|---|---|---|---|---|---|---|---|---|---|---|
| ANISOU | 851 | C | ASP A | 502 | 3137 | 2397 | 3122 | 63 | 51 | 35 | C |

| | | | | | | | | | | |
|---|---|---|---|---|---|---|---|---|---|---|
| ATOM | 852 | O | ASP A | 502 | 13.750 | 10.911 | 81.856 | 1.00 | 21.88 | O |

| | | | | | | | | | | | |
|---|---|---|---|---|---|---|---|---|---|---|---|
| ANISOU | 952 | O | ASP A | 502 | 3058 | 2139 | 3117 | −149 | 117 | 149 | O |

| | | | | | | | | | | |
|---|---|---|---|---|---|---|---|---|---|---|
| ATOM | 853 | CB | ASP A | 502 | 16.075 | 8.708 | 81.193 | 1.00 | 28.57 | C |

| | | | | | | | | | | | |
|---|---|---|---|---|---|---|---|---|---|---|---|
| ANISOU | 853 | CB | ASP A | 502 | 3591 | 3354 | 3910 | 30 | −109 | 48 | C |

| | | | | | | | | | | |
|---|---|---|---|---|---|---|---|---|---|---|
| ATOM | 854 | CG | ASP A | 502 | 16.387 | 7.220 | 81.350 | 1.00 | 32.24 | C |

| | | | | | | | | | | | |
|---|---|---|---|---|---|---|---|---|---|---|---|
| ANISOU | 854 | CG | ASP A | 502 | 4240 | 3520 | 4489 | 122 | 5 | 10 | C |

| | | | | | | | | | | |
|---|---|---|---|---|---|---|---|---|---|---|
| ATOM | 855 | OD1 | ASP A | 502 | 15.442 | 6.425 | 81.379 | 1.00 | 33.56 | O |

| | | | | | | | | | | | |
|---|---|---|---|---|---|---|---|---|---|---|---|
| ANISOU | 855 | OD1 | ASP A | 502 | 4614 | 3436 | 4700 | −46 | −5 | 86 | O |

| | | | | | | | | | | |
|---|---|---|---|---|---|---|---|---|---|---|
| ATOM | 856 | OD2 | ASP A | 502 | 17.583 | 6.891 | 81.441 | 1.00 | 34.47 | O |

| | | | | | | | | | | | |
|---|---|---|---|---|---|---|---|---|---|---|---|
| ANISOU | 856 | OD2 | ASP A | 502 | 4372 | 3836 | 4889 | 111 | −107 | 27 | O |

| | | | | | | | | | | |
|---|---|---|---|---|---|---|---|---|---|---|
| ATOM | 857 | N | LYS A | 503 | 15.003 | 11.306 | 80.043 | 1.00 | 21.76 | N |

| | | | | | | | | | | | |
|---|---|---|---|---|---|---|---|---|---|---|---|
| ANISOU | 857 | N | LYS A | 503 | 2816 | 2231 | 3221 | 62 | −61 | 75 | N |

| | | | | | | | | | | |
|---|---|---|---|---|---|---|---|---|---|---|
| ATOM | 858 | CA | LYS A | 503 | 14.861 | 12.766 | 80.175 | 1.00 | 20.78 | C |

| | | | | | | | | | | | |
|---|---|---|---|---|---|---|---|---|---|---|---|
| ANISOU | 858 | CA | LYS A | 503 | 2871 | 2240 | 2786 | −100 | −77 | −39 | C |

| | | | | | | | | | | |
|---|---|---|---|---|---|---|---|---|---|---|
| ATOM | 859 | C | LYS A | 503 | 14.722 | 13.436 | 78.822 | 1.00 | 19.90 | C |

| | | | | | | | | | | | |
|---|---|---|---|---|---|---|---|---|---|---|---|
| ANISOU | 859 | C | LYS A | 503 | 2611 | 2113 | 2836 | −163 | 51 | 4 | C |

| | | | | | | | | | | |
|---|---|---|---|---|---|---|---|---|---|---|
| ATOM | 860 | O | LYS A | 503 | 15.260 | 12.961 | 77.820 | 1.00 | 19.92 | O |

| | | | | | | | | | | | |
|---|---|---|---|---|---|---|---|---|---|---|---|
| ANISOU | 860 | O | LYS A | 503 | 2563 | 2046 | 2959 | −20 | 12 | −67 | O |

| | | | | | | | | | | |
|---|---|---|---|---|---|---|---|---|---|---|
| ATOM | 861 | CB | LYS A | 503 | 16.095 | 13.375 | 80.863 | 1.00 | 21.99 | C |

| | | | | | | | | | | | |
|---|---|---|---|---|---|---|---|---|---|---|---|
| ANISOU | 861 | CB | LYS A | 503 | 2829 | 2545 | 2980 | −89 | −107 | 42 | C |

| | | | | | | | | | | |
|---|---|---|---|---|---|---|---|---|---|---|
| ATOM | 862 | CG | LYS A | 503 | 16.353 | 12.959 | 82.295 | 1.00 | 23.57 | C |

| | | | | | | | | | | | |
|---|---|---|---|---|---|---|---|---|---|---|---|
| ANISOU | 862 | CG | LYS A | 503 | 3179 | 2755 | 3020 | −85 | −174 | 118 | C |

| | | | | | | | | | | |
|---|---|---|---|---|---|---|---|---|---|---|
| ATOM | 863 | CD | LYS A | 503 | 17.372 | 13.907 | 82.936 | 1.00 | 25.83 | C |

| | | | | | | | | | | | |
|---|---|---|---|---|---|---|---|---|---|---|---|
| ANISOU | 863 | CD | LYS A | 503 | 3183 | 3249 | 3383 | −302 | −178 | 36 | C |

| | | | | | | | | | | |
|---|---|---|---|---|---|---|---|---|---|---|
| ATOM | 864 | CE | LYS A | 503 | 17.693 | 13.448 | 84.353 | 1.00 | 27.13 | C |

| | | | | | | | | | | | |
|---|---|---|---|---|---|---|---|---|---|---|---|
| ANISOU | 864 | CE | LYS A | 503 | 3477 | 3464 | 3368 | −216 | −19 | 80 | C |

| | | | | | | | | | | |
|---|---|---|---|---|---|---|---|---|---|---|
| ATOM | 865 | NZ | LYS A | 503 | 18.191 | 12.036 | 84.332 | 1.00 | 28.53 | N |

| | | | | | | | | | | | |
|---|---|---|---|---|---|---|---|---|---|---|---|
| ANISOU | 865 | NZ | LYS A | 503 | 3575 | 3704 | 3560 | −20 | −24 | 19 | N |

| | | | | | | | | | | |
|---|---|---|---|---|---|---|---|---|---|---|
| ATOM | 866 | N | ILE A | 504 | 14.062 | 14.597 | 78.796 | 1.00 | 19.58 | N |

| | | | | | | | | | | | |
|---|---|---|---|---|---|---|---|---|---|---|---|
| ANISOU | 866 | N | ILE A | 504 | 2452 | 2229 | 2759 | −68 | 20 | 111 | N |

| | | | | | | | | | | |
|---|---|---|---|---|---|---|---|---|---|---|
| ATOM | 867 | CA | ILE A | 504 | 14.024 | 15.466 | 77.619 | 1.00 | 19.71 | C |

| | | | | | | | | | | | |
|---|---|---|---|---|---|---|---|---|---|---|---|
| ANISOU | 867 | CA | ILE A | 504 | 2612 | 2159 | 2719 | −139 | −139 | 13 | C |

| | | | | | | | | | | |
|---|---|---|---|---|---|---|---|---|---|---|
| ATOM | 868 | C | ILE A | 504 | 14.460 | 16.861 | 78.108 | 1.00 | 19.00 | C |

| | | | | | | | | | | | |
|---|---|---|---|---|---|---|---|---|---|---|---|
| ANISOU | 868 | C | ILE A | 504 | 2454 | 2130 | 2637 | −222 | 126 | −4 | C |

| | | | | | | | | | | |
|---|---|---|---|---|---|---|---|---|---|---|
| ATOM | 869 | O | ILE A | 504 | 13.856 | 17.312 | 79.096 | 1.00 | 18.32 | O |

| | | | | | | | | | | | |
|---|---|---|---|---|---|---|---|---|---|---|---|
| ANISOU | 869 | O | ILE A | 504 | 2545 | 1805 | 2610 | −267 | 180 | 13 | O |

| | | | | | | | | | | |
|---|---|---|---|---|---|---|---|---|---|---|
| ATOM | 870 | CB | ILE A | 504 | 12.625 | 15.642 | 77.006 | 1.00 | 19.18 | C |

| | | | | | | | | | | | |
|---|---|---|---|---|---|---|---|---|---|---|---|
| ANISOU | 870 | CB | ILE A | 504 | 2403 | 2188 | 2698 | −122 | −104 | 3 | C |

| | | | | | | | | | | |
|---|---|---|---|---|---|---|---|---|---|---|
| ATOM | 871 | CG1 | ILE A | 504 | 12.186 | 14.292 | 76.412 | 1.00 | 21.05 | C |

| | | | | | | | | | | | |
|---|---|---|---|---|---|---|---|---|---|---|---|
| ANISOU | 871 | CG1 | ILE A | 504 | 2649 | 2366 | 2982 | −206 | −150 | −105 | C |

| | | | | | | | | | | |
|---|---|---|---|---|---|---|---|---|---|---|
| ATOM | 872 | CG2 | ILE A | 504 | 12.704 | 16.623 | 75.823 | 1.00 | 20.43 | C |

| | | | | | | | | | | | |
|---|---|---|---|---|---|---|---|---|---|---|---|
| ANISOU | 872 | CG2 | ILE A | 504 | 2621 | 2359 | 2781 | −105 | −96 | 79 | C |

| | | | | | | | | | | |
|---|---|---|---|---|---|---|---|---|---|---|
| ATOM | 873 | CD1 | ILE A | 504 | 10.689 | 14.246 | 76.137 | 1.00 | 21.81 | C |

| | | | | | | | | | | | |
|---|---|---|---|---|---|---|---|---|---|---|---|
| ANISOU | 873 | CD1 | ILE A | 504 | 2487 | 2600 | 3200 | −45 | 35 | −14 | C |

| | | | | | | | | | | |
|---|---|---|---|---|---|---|---|---|---|---|
| ATOM | 874 | N | PHE A | 505 | 15.478 | 17.432 | 77.489 | 1.00 | 16.76 | N |

| | | | | | | | | | | | |
|---|---|---|---|---|---|---|---|---|---|---|---|
| ANISOU | 874 | N | PHE A | 505 | 2350 | 1723 | 2294 | −112 | 50 | 121 | N |

| | | | | | | | | | | |
|---|---|---|---|---|---|---|---|---|---|---|
| ATOM | 875 | CA | PHE A | 505 | 15.939 | 18.778 | 77.885 | 1.00 | 15.92 | C |

| | | | | | | | | | | | |
|---|---|---|---|---|---|---|---|---|---|---|---|
| ANISOU | 875 | CA | PHE A | 505 | 1984 | 1899 | 2165 | −26 | −102 | −79 | C |

| | | | | | | | | | | |
|---|---|---|---|---|---|---|---|---|---|---|
| ATOM | 876 | C | PHE A | 505 | 15.418 | 19.798 | 76.892 | 1.00 | 17.51 | C |

| | | | | | | | | | | | |
|---|---|---|---|---|---|---|---|---|---|---|---|
| ANISOU | 876 | C | PHE A | 505 | 2517 | 1855 | 2282 | −157 | 31 | 10 | C |

| | | | | | | | | | | |
|---|---|---|---|---|---|---|---|---|---|---|
| ATOM | 877 | O | PHE A | 505 | 15.458 | 19.531 | 75.680 | 1.00 | 18.22 | O |

| | | | | | | | | | | | |
|---|---|---|---|---|---|---|---|---|---|---|---|
| ANISOU | 877 | O | PHE A | 505 | 2693 | 1810 | 2418 | 13 | 93 | −40 | O |

| | | | | | | | | | | |
|---|---|---|---|---|---|---|---|---|---|---|
| ATOM | 878 | CB | PHE A | 505 | 17.497 | 18.864 | 77.935 | 1.00 | 15.84 | C |

| | | | | | | | | | | | |
|---|---|---|---|---|---|---|---|---|---|---|---|
| ANISOU | 878 | CB | PHE A | 505 | 2001 | 1798 | 2218 | −97 | 50 | 95 | C |

| | | | | | | | | | | |
|---|---|---|---|---|---|---|---|---|---|---|
| ATOM | 879 | CG | PHE A | 505 | 18.027 | 18.114 | 79.135 | 1.00 | 17.42 | C |

| | | | | | | | | | | | |
|---|---|---|---|---|---|---|---|---|---|---|---|
| ANISOU | 879 | CG | PHE A | 505 | 2451 | 1851 | 2318 | −81 | −10 | 52 | C |

| | | | | | | | | | | |
|---|---|---|---|---|---|---|---|---|---|---|
| ATOM | 880 | CD1 | PHE A | 505 | 18.435 | 16.796 | 78.952 | 1.00 | 18.07 | C |

| | | | | | | | | | | | |
|---|---|---|---|---|---|---|---|---|---|---|---|
| ANISOU | 880 | CD1 | PHE A | 505 | 2540 | 1921 | 2405 | 88 | 63 | 127 | C |

| | | | | | | | | | | |
|---|---|---|---|---|---|---|---|---|---|---|
| ATOM | 881 | CD2 | PHE A | 505 | 18.180 | 18.693 | 80.378 | 1.00 | 17.14 | C |

| | | | | | | | | | | | |
|---|---|---|---|---|---|---|---|---|---|---|---|
| ANISOU | 881 | CD2 | PHE A | 505 | 2124 | 2146 | 2242 | −31 | 127 | 75 | C |

| | | | | | | | | | | |
|---|---|---|---|---|---|---|---|---|---|---|
| ATOM | 882 | CE1 | PHE A | 505 | 18.948 | 16.092 | 80.033 | 1.00 | 18.58 | C |

| | | | | | | | | | | | |
|---|---|---|---|---|---|---|---|---|---|---|---|
| ANISOU | 882 | CE1 | PHE A | 505 | 2396 | 2231 | 2434 | −67 | −149 | 100 | C |

| | | | | | | | | | | |
|---|---|---|---|---|---|---|---|---|---|---|
| ATOM | 883 | CE2 | PHE A | 505 | 18.672 | 17.998 | 81.465 | 1.00 | 19.19 | C |

| | | | | | | | | | | | |
|---|---|---|---|---|---|---|---|---|---|---|---|
| ANISOU | 883 | CE2 | PHE A | 505 | 2488 | 2224 | 2580 | 85 | −73 | 37 | C |

| | | | | | | | | | | |
|---|---|---|---|---|---|---|---|---|---|---|
| ATOM | 884 | CZ | PHE A | 505 | 19.076 | 16.666 | 81.285 | 1.00 | 19.09 | C |

| | | | | | | | | | | | |
|---|---|---|---|---|---|---|---|---|---|---|---|
| ANISOU | 884 | CZ | PHE A | 505 | 2543 | 2145 | 2567 | −38 | 14 | −21 | C |

| | | | | | | | | | | |
|---|---|---|---|---|---|---|---|---|---|---|
| ATOM | 885 | N | THR A | 506 | 14.924 | 20.959 | 77.350 | 1.00 | 16.31 | N |

| | | | | | | | | | | | |
|---|---|---|---|---|---|---|---|---|---|---|---|
| ANISOU | 885 | N | THR A | 506 | 1966 | 1857 | 2373 | 65 | −100 | 195 | N |

| | | | | | | | | | | |
|---|---|---|---|---|---|---|---|---|---|---|
| ATOM | 886 | CA | THR A | 506 | 14.430 | 21.983 | 76.447 | 1.00 | 17.01 | C |

| | | | | | | | | | | | |
|---|---|---|---|---|---|---|---|---|---|---|---|
| ANISOU | 886 | CA | THR A | 506 | 2326 | 1849 | 2288 | −61 | −134 | 102 | C |

| | | | | | | | | | | |
|---|---|---|---|---|---|---|---|---|---|---|
| ATOM | 887 | C | THR A | 506 | 15.000 | 23.342 | 76.863 | 1.00 | 16.40 | C |

| | | | | | | | | | | | |
|---|---|---|---|---|---|---|---|---|---|---|---|
| ANISOU | 887 | C | THR A | 506 | 2127 | 1829 | 2274 | 28 | −2 | 21 | C |

| | | | | | | | | | | |
|---|---|---|---|---|---|---|---|---|---|---|
| ATOM | 888 | O | THR A | 506 | 14.522 | 23.974 | 77.846 | 1.00 | 17.48 | O |

| | | | | | | | | | | | |
|---|---|---|---|---|---|---|---|---|---|---|---|
| ANISOU | 888 | O | THR A | 506 | 2409 | 1929 | 2306 | −19 | 19 | 23 | O |

| | | | | | | | | | | |
|---|---|---|---|---|---|---|---|---|---|---|
| ATOM | 889 | CB | THR A | 506 | 12.872 | 22.057 | 76.476 | 1.00 | 17.01 | C |

| | | | | | | | | | | | |
|---|---|---|---|---|---|---|---|---|---|---|---|
| ANISOU | 889 | CB | THR A | 506 | 2206 | 1853 | 2404 | −153 | 70 | −69 | C |

| | | | | | | | | | | |
|---|---|---|---|---|---|---|---|---|---|---|
| ATOM | 890 | OG1 | THR A | 506 | 12.431 | 22.374 | 77.820 | 1.00 | 18.67 | O |

| | | | | | | | | | | | |
|---|---|---|---|---|---|---|---|---|---|---|---|
| ANISOU | 890 | OG1 | THR A | 506 | 2514 | 2077 | 2504 | −154 | 166 | −42 | O |

| | | | | | | | | | | |
|---|---|---|---|---|---|---|---|---|---|---|
| ATOM | 891 | CG2 | THR A | 506 | 12.276 | 20.709 | 76.080 | 1.00 | 18.31 | C |

| | | | | | | | | | | | |
|---|---|---|---|---|---|---|---|---|---|---|---|
| ANISOU | 891 | CG2 | THR A | 506 | 2451 | 1842 | 2664 | −101 | −151 | −38 | C |

| | | | | | | | | | | |
|---|---|---|---|---|---|---|---|---|---|---|
| ATOM | 892 | N | PRO A | 507 | 16.086 | 23.783 | 76.275 | 1.00 | 16.35 | N |

| | | | | | | | | | | | |
|---|---|---|---|---|---|---|---|---|---|---|---|
| ANISOU | 892 | N | PRO A | 507 | 2260 | 1805 | 2145 | −18 | 52 | −32 | N |

| | | | | | | | | | | |
|---|---|---|---|---|---|---|---|---|---|---|
| ATOM | 893 | CA | PRO A | 507 | 16.660 | 25.108 | 76.490 | 1.00 | 15.56 | C |

| | | | | | | | | | | | |
|---|---|---|---|---|---|---|---|---|---|---|---|
| ANISOU | 893 | CA | PRO A | 507 | 1998 | 1800 | 2115 | 101 | 38 | −174 | C |

| | | | | | | | | | | |
|---|---|---|---|---|---|---|---|---|---|---|
| ATOM | 894 | C | PRO A | 507 | 15.641 | 26.138 | 75.973 | 1.00 | 15.81 | C |

| | | | | | | | | | | | |
|---|---|---|---|---|---|---|---|---|---|---|---|
| ANISOU | 894 | C | PRO A | 507 | 2151 | 1760 | 2096 | −73 | 0 | −12 | C |

| | | | | | | | | | | |
|---|---|---|---|---|---|---|---|---|---|---|
| ATOM | 895 | O | PRO A | 507 | 14.941 | 25.945 | 74.979 | 1.00 | 16.44 | O |

| | | | | | | | | | | | |
|---|---|---|---|---|---|---|---|---|---|---|---|
| ANISOU | 895 | O | PRO A | 507 | 2347 | 1690 | 2208 | 58 | 8 | 190 | O |

| | | | | | | | | | | |
|---|---|---|---|---|---|---|---|---|---|---|
| ATOM | 896 | CB | PRO A | 507 | 17.963 | 25.133 | 75.657 | 1.00 | 16.57 | C |

| | | | | | | | | | | | |
|---|---|---|---|---|---|---|---|---|---|---|---|
| ANISOU | 896 | CB | PRO A | 507 | 2099 | 2133 | 2063 | −193 | 52 | −133 | C |

| | | | | | | | | | | |
|---|---|---|---|---|---|---|---|---|---|---|
| ATOM | 897 | CG | PRO A | 507 | 17.661 | 24.174 | 74.537 | 1.00 | 16.13 | C |

| | | | | | | | | | | | |
|---|---|---|---|---|---|---|---|---|---|---|---|
| ANISOU | 897 | CG | PRO A | 507 | 2028 | 1997 | 2102 | −232 | 39 | −127 | C |

| | | | | | | | | | | |
|---|---|---|---|---|---|---|---|---|---|---|
| ATOM | 898 | CD | PRO A | 507 | 16.795 | 23.082 | 75.166 | 1.00 | 16.06 | C |

| | | | | | | | | | | | |
|---|---|---|---|---|---|---|---|---|---|---|---|
| ANISOU | 898 | CD | PRO A | 507 | 1964 | 2117 | 2022 | −145 | 170 | −38 | C |

| | | | | | | | | | | |
|---|---|---|---|---|---|---|---|---|---|---|
| ATOM | 899 | N | ILE A | 508 | 15.512 | 27.211 | 76.780 | 1.00 | 15.49 | N |

| | | | | | | | | | | | |
|---|---|---|---|---|---|---|---|---|---|---|---|
| ANISOU | 899 | N | ILE A | 508 | 1821 | 1590 | 2476 | 207 | 94 | 21 | N |

| | | | | | | | | | | |
|---|---|---|---|---|---|---|---|---|---|---|
| ATOM | 900 | CA | ILE A | 508 | 14.492 | 28.241 | 76.501 | 1.00 | 16.06 | C |

| | | | | | | | | | | | |
|---|---|---|---|---|---|---|---|---|---|---|---|
| ANISOU | 900 | CA | ILE A | 508 | 2189 | 1537 | 2375 | 146 | −32 | 66 | C |

| | | | | | | | | | | |
|---|---|---|---|---|---|---|---|---|---|---|
| ATOM | 901 | C | ILE A | 508 | 15.104 | 29.627 | 76.342 | 1.00 | 14.76 | C |

| | | | | | | | | | | | |
|---|---|---|---|---|---|---|---|---|---|---|---|
| ANISOU | 901 | C | ILE A | 508 | 1768 | 1682 | 2160 | 75 | 84 | 81 | C |

| | | | | | | | | | | |
|---|---|---|---|---|---|---|---|---|---|---|
| ATOM | 902 | O | ILE A | 508 | 15.881 | 30.041 | 77.202 | 1.00 | 16.01 | O |

| | | | | | | | | | | | |
|---|---|---|---|---|---|---|---|---|---|---|---|
| ANISOU | 902 | O | ILE A | 508 | 2335 | 1501 | 2246 | 42 | 125 | 0 | O |

| | | | | | | | | | | |
|---|---|---|---|---|---|---|---|---|---|---|
| ATOM | 903 | CB | ILE A | 508 | 13.540 | 28.264 | 77.698 | 1.00 | 17.17 | C |

| | | | | | | | | | | | |
|---|---|---|---|---|---|---|---|---|---|---|---|
| ANISOU | 903 | CB | ILE A | 508 | 1886 | 1924 | 2713 | 101 | 72 | 89 | C |

| | | | | | | | | | | |
|---|---|---|---|---|---|---|---|---|---|---|
| ATOM | 904 | CG1 | ILE A | 508 | 12.766 | 26.906 | 77.805 | 1.00 | 18.68 | C |

| | | | | | | | | | | | |
|---|---|---|---|---|---|---|---|---|---|---|---|
| ANISOU | 904 | CG1 | ILE A | 508 | 2437 | 2058 | 2603 | 49 | 182 | 162 | C |

| | | | | | | | | | | |
|---|---|---|---|---|---|---|---|---|---|---|
| ATOM | 905 | CD2 | ILE A | 508 | 12.512 | 29.416 | 77.577 | 1.00 | 18.32 | C |

| | | | | | | | | | | | |
|---|---|---|---|---|---|---|---|---|---|---|---|
| ANISOU | 905 | CD2 | ILE A | 508 | 2289 | 1951 | 2720 | 288 | 255 | 89 | C |

| | | | | | | | | | | |
|---|---|---|---|---|---|---|---|---|---|---|
| ATOM | 906 | CD1 | ILE A | 508 | 12.105 | 26.794 | 79.167 | 1.00 | 21.03 | C |

| | | | | | | | | | | | |
|---|---|---|---|---|---|---|---|---|---|---|---|
| ANISOU | 906 | CD1 | ILE A | 508 | 2779 | 2685 | 2525 | 165 | 159 | 13 | C |

| | | | | | | | | | | |
|---|---|---|---|---|---|---|---|---|---|---|
| ATOM | 907 | N | ASN A | 509 | 14.769 | 30.269 | 75.213 | 1.00 | 16.29 | N |

| | | | | | | | | | | | |
|---|---|---|---|---|---|---|---|---|---|---|---|
| ANISOU | 907 | N | ASN A | 509 | 2216 | 1847 | 2125 | 67 | 11 | 41 | N |

| | | | | | | | | | | |
|---|---|---|---|---|---|---|---|---|---|---|
| ATOM | 908 | CA | ASN A | 509 | 15.243 | 31.625 | 74.991 | 1.00 | 16.80 | C |

| | | | | | | | | | | | |
|---|---|---|---|---|---|---|---|---|---|---|---|
| ANISOU | 908 | CA | ASN A | 509 | 2221 | 1928 | 2234 | −35 | −23 | −43 | C |

| | | | | | | | | | | |
|---|---|---|---|---|---|---|---|---|---|---|
| ATOM | 909 | C | ASN A | 509 | 14.106 | 32.539 | 75.453 | 1.00 | 18.14 | C |

| | | | | | | | | | | | |
|---|---|---|---|---|---|---|---|---|---|---|---|
| ANISOU | 909 | C | ASN A | 509 | 2297 | 2141 | 2454 | 57 | −4 | −80 | C |

| | | | | | | | | | | |
|---|---|---|---|---|---|---|---|---|---|---|
| ATOM | 910 | O | ASN A | 509 | 12.941 | 32.250 | 75.139 | 1.00 | 21.01 | O |

| | | | | | | | | | | | |
|---|---|---|---|---|---|---|---|---|---|---|---|
| ANISOU | 910 | O | ASN A | 509 | 2471 | 2365 | 3147 | 63 | −232 | −141 | O |

| | | | | | | | | | | |
|---|---|---|---|---|---|---|---|---|---|---|
| ATOM | 911 | CB | ASN A | 509 | 15.453 | 31.934 | 73.507 | 1.00 | 19.29 | C |

| | | | | | | | | | | | |
|---|---|---|---|---|---|---|---|---|---|---|---|
| ANISOU | 911 | CB | ASN A | 509 | 2709 | 2328 | 2291 | 14 | 17 | 101 | C |

| | | | | | | | | | | |
|---|---|---|---|---|---|---|---|---|---|---|
| ATOM | 912 | CG | ASN A | 509 | 16.026 | 33.346 | 73.388 | 1.00 | 19.06 | C |

| | | | | | | | | | | | |
|---|---|---|---|---|---|---|---|---|---|---|---|
| ANISOU | 912 | CG | ASN A | 509 | 2477 | 2244 | 2520 | 53 | −147 | 69 | C |

| | | | | | | | | | | |
|---|---|---|---|---|---|---|---|---|---|---|
| ATOM | 913 | OD1 | ASN A | 509 | 15.293 | 34.259 | 72.963 | 1.00 | 20.78 | O |

| | | | | | | | | | | | |
|---|---|---|---|---|---|---|---|---|---|---|---|
| ANISOU | 913 | OD1 | ASN A | 509 | 2749 | 2310 | 2836 | 182 | −291 | 250 | O |

| | | | | | | | | | | |
|---|---|---|---|---|---|---|---|---|---|---|
| ATOM | 914 | ND2 | ASN A | 509 | 17.304 | 33.535 | 73.676 | 1.00 | 18.78 | N |

| | | | | | | | | | | | |
|---|---|---|---|---|---|---|---|---|---|---|---|
| ANISOU | 914 | ND2 | ASN A | 509 | 2533 | 2406 | 2195 | 77 | −80 | 262 | N |

| | | | | | | | | | | |
|---|---|---|---|---|---|---|---|---|---|---|
| ATOM | 915 | N | LEU A | 510 | 14.429 | 33.498 | 76.303 | 1.00 | 18.10 | N |

| | | | | | | | | | | | |
|---|---|---|---|---|---|---|---|---|---|---|---|
| ANISOU | 915 | N | LEU A | 510 | 2188 | 2146 | 2542 | 126 | −134 | −165 | N |

| | | | | | | | | | | |
|---|---|---|---|---|---|---|---|---|---|---|
| ATOM | 916 | CA | LEU A | 510 | 13.405 | 34.404 | 76.826 | 1.00 | 20.12 | C |

| | | | | | | | | | | | |
|---|---|---|---|---|---|---|---|---|---|---|---|
| ANISOU | 916 | CA | LEU A | 510 | 2447 | 2387 | 2812 | 150 | 82 | −51 | C |

| | | | | | | | | | | |
|---|---|---|---|---|---|---|---|---|---|---|
| ATOM | 917 | C | LEU A | 510 | 13.677 | 35.792 | 76.249 | 1.00 | 22.23 | C |

| | | | | | | | | | | | |
|---|---|---|---|---|---|---|---|---|---|---|---|
| ANISOU | 917 | C | LEU A | 510 | 2851 | 2477 | 3117 | 9 | 62 | 5 | C |

| | | | | | | | | | | |
|---|---|---|---|---|---|---|---|---|---|---|
| ATOM | 918 | O | LEU A | 510 | 14.658 | 36.428 | 76.654 | 1.00 | 21.24 | O |

| | | | | | | | | | | | |
|---|---|---|---|---|---|---|---|---|---|---|---|
| ANISOU | 918 | O | LEU A | 510 | 2656 | 2501 | 2913 | 83 | 171 | 201 | O |

| | | | | | | | | | | |
|---|---|---|---|---|---|---|---|---|---|---|
| ATOM | 919 | CB | LEU A | 510 | 13.497 | 34.430 | 78.351 | 1.00 | 22.33 | C |

| | | | | | | | | | | | |
|---|---|---|---|---|---|---|---|---|---|---|---|
| ANISOU | 919 | CB | LEU A | 510 | 2925 | 2691 | 2867 | 214 | 167 | −78 | C |

| | | | | | | | | | | |
|---|---|---|---|---|---|---|---|---|---|---|
| ATOM | 920 | CG | LEU A | 510 | 13.138 | 33.057 | 78.971 | 1.00 | 25.04 | C |

| | | | | | | | | | | | |
|---|---|---|---|---|---|---|---|---|---|---|---|
| ANISOU | 920 | CG | LEU A | 510 | 3471 | 2861 | 3181 | −72 | 84 | 5 | C |

| | | | | | | | | | | |
|---|---|---|---|---|---|---|---|---|---|---|
| ATOM | 921 | CD1 | LEU A | 510 | 13.746 | 32.940 | 80.370 | 1.00 | 27.03 | C |

| | | | | | | | | | | | |
|---|---|---|---|---|---|---|---|---|---|---|---|
| ANISOU | 921 | CD1 | LEU A | 510 | 3670 | 3299 | 3300 | −117 | −12 | 95 | C |

| | | | | | | | | | | |
|---|---|---|---|---|---|---|---|---|---|---|
| ATOM | 922 | CD2 | LEU A | 510 | 11.649 | 32.824 | 79.011 | 1.00 | 26.25 | C |

| | | | | | | | | | | | |
|---|---|---|---|---|---|---|---|---|---|---|---|
| ANISOU | 922 | CD2 | LEU A | 510 | 3524 | 3043 | 3407 | −11 | 8 | 74 | C |

| | | | | | | | | | | |
|---|---|---|---|---|---|---|---|---|---|---|
| ATOM | 923 | N | ASN A | 511 | 12.822 | 36.222 | 75.336 | 1.00 | 23.67 | N |

| | | | | | | | | | | | |
|---|---|---|---|---|---|---|---|---|---|---|---|
| ANISOU | 923 | N | ASN A | 511 | 3078 | 2707 | 3207 | 14 | −111 | 36 | N |

| | | | | | | | | | | |
|---|---|---|---|---|---|---|---|---|---|---|
| ATOM | 924 | CA | ASN A | 511 | 12.839 | 37.585 | 74.828 | 1.00 | 26.61 | C |

| | | | | | | | | | | | |
|---|---|---|---|---|---|---|---|---|---|---|---|
| ANISOU | 924 | CA | ASN A | 511 | 3453 | 3017 | 3643 | −8 | 51 | 284 | C |

| | | | | | | | | | | |
|---|---|---|---|---|---|---|---|---|---|---|
| ATOM | 925 | C | ASN A | 511 | 14.140 | 38.077 | 74.259 | 1.00 | 24.33 | C |

| | | | | | | | | | | | |
|---|---|---|---|---|---|---|---|---|---|---|---|
| ANISOU | 925 | C | ASN A | 511 | 3173 | 2674 | 3396 | 111 | −34 | 7 | C |

| | | | | | | | | | | |
|---|---|---|---|---|---|---|---|---|---|---|
| ATOM | 926 | O | ASN A | 511 | 14.493 | 39.265 | 74.368 | 1.00 | 23.65 | O |

| | | | | | | | | | | | |
|---|---|---|---|---|---|---|---|---|---|---|---|
| ANISOU | 926 | O | ASN A | 511 | 3162 | 2356 | 3470 | 367 | 44 | 32 | O |

| | | | | | | | | | | |
|---|---|---|---|---|---|---|---|---|---|---|
| ATOM | 927 | CB | ASN A | 511 | 12.494 | 38.544 | 76.000 | 1.00 | 29.89 | C |

| | | | | | | | | | | | |
|---|---|---|---|---|---|---|---|---|---|---|---|
| ANISOU | 927 | CB | ASN A | 511 | 4028 | 3487 | 3842 | 116 | −38 | 62 | C |

| | | | | | | | | | | |
|---|---|---|---|---|---|---|---|---|---|---|
| ATOM | 928 | CG | ASN A | 511 | 11.173 | 38.196 | 76.663 | 1.00 | 34.64 | C |

| | | | | | | | | | | | |
|---|---|---|---|---|---|---|---|---|---|---|---|
| ANISOU | 928 | CG | ASN A | 511 | 4283 | 4313 | 4568 | −107 | 140 | 89 | C |

| | | | | | | | | | | |
|---|---|---|---|---|---|---|---|---|---|---|
| ATOM | 929 | OD1 | ASN A | 511 | 10.153 | 38.126 | 75.973 | 1.00 | 36.77 | O |

| | | | | | | | | | | | |
|---|---|---|---|---|---|---|---|---|---|---|---|
| ANISOU | 929 | OD1 | ASN A | 511 | 4452 | 4802 | 4717 | −42 | −19 | 45 | O |

| | | | | | | | | | | |
|---|---|---|---|---|---|---|---|---|---|---|
| ATOM | 930 | ND2 | ASN A | 511 | 11.208 | 37.969 | 77.978 | 1.00 | 37.35 | N |

| | | | | | | | | | | | |
|---|---|---|---|---|---|---|---|---|---|---|---|
| ANISOU | 930 | ND2 | ASN A | 511 | 4792 | 4745 | 4656 | −83 | −9 | 34 | N |

| | | | | | | | | | | |
|---|---|---|---|---|---|---|---|---|---|---|
| ATOM | 931 | N | GLN A | 512 | 14.927 | 37.179 | 73.659 | 1.00 | 22.30 | N |

| | | | | | | | | | | | |
|---|---|---|---|---|---|---|---|---|---|---|---|
| ANISOU | 931 | N | GLN A | 512 | 2758 | 2502 | 3211 | 158 | −22 | 256 | N |

| | | | | | | | | | | |
|---|---|---|---|---|---|---|---|---|---|---|
| ATOM | 932 | CA | GLN A | 512 | 16.230 | 37.545 | 73.105 | 1.00 | 21.51 | C |

| | | | | | | | | | | | |
|---|---|---|---|---|---|---|---|---|---|---|---|
| ANISOU | 932 | CA | GLN A | 512 | 2922 | 2385 | 2868 | 128 | 51 | 214 | C |

| | | | | | | | | | | |
|---|---|---|---|---|---|---|---|---|---|---|
| ATOM | 933 | C | GLN A | 512 | 17.133 | 38.170 | 74.131 | 1.00 | 21.94 | C |

| | | | | | | | | | | | |
|---|---|---|---|---|---|---|---|---|---|---|---|
| ANISOU | 933 | C | GLN A | 512 | 2912 | 2471 | 2953 | 318 | 0 | 88 | C |

| | | | | | | | | | | |
|---|---|---|---|---|---|---|---|---|---|---|
| ATOM | 934 | O | GLN A | 512 | 18.072 | 38.889 | 73.731 | 1.00 | 22.45 | O |

| | | | | | | | | | | | |
|---|---|---|---|---|---|---|---|---|---|---|---|
| ANISOU | 934 | O | GLN A | 512 | 3027 | 2550 | 2951 | 175 | −17 | 119 | O |

| | | | | | | | | | | |
|---|---|---|---|---|---|---|---|---|---|---|
| ATOM | 935 | CB | GLN A | 512 | 16.055 | 38.478 | 71.882 | 1.00 | 21.79 | C |

| | | | | | | | | | | | |
|---|---|---|---|---|---|---|---|---|---|---|---|
| ANISOU | 935 | CB | GLN A | 512 | 3083 | 2399 | 2817 | 76 | 74 | 215 | C |

| | | | | | | | | | | |
|---|---|---|---|---|---|---|---|---|---|---|
| ATOM | 936 | CG | GLN A | 512 | 15.366 | 37.787 | 70.728 | 1.00 | 23.35 | C |

| | | | | | | | | | | | |
|---|---|---|---|---|---|---|---|---|---|---|---|
| ANISOU | 936 | CG | GLN A | 512 | 3270 | 2775 | 2827 | 115 | 10 | 138 | C |

| | | | | | | | | | | |
|---|---|---|---|---|---|---|---|---|---|---|
| ATOM | 937 | CD | GLN A | 512 | 16.254 | 36.895 | 69.898 | 1.00 | 22.57 | C |

| | | | | | | | | | | | |
|---|---|---|---|---|---|---|---|---|---|---|---|
| ANISOU | 937 | CD | GLN A | 512 | 2948 | 2714 | 2914 | 17 | −43 | 101 | C |

| | | | | | | | | | | |
|---|---|---|---|---|---|---|---|---|---|---|
| ATOM | 938 | OE1 | GLN A | 512 | 17.459 | 36.829 | 70.152 | 1.00 | 23.69 | O |

| | | | | | | | | | | | |
|---|---|---|---|---|---|---|---|---|---|---|---|
| ANISOU | 938 | OE1 | GLN A | 512 | 3046 | 2928 | 3029 | 403 | −92 | −177 | O |

| | | | | | | | | | | |
|---|---|---|---|---|---|---|---|---|---|---|
| ATOM | 939 | NE2 | GLN A | 512 | 15.721 | 36.260 | 68.873 | 1.00 | 22.83 | N |

| | | | | | | | | | | | |
|---|---|---|---|---|---|---|---|---|---|---|---|
| ANISOU | 939 | NE2 | GLN A | 512 | 3131 | 2652 | 2890 | 97 | −267 | 229 | N |

| | | | | | | | | | | |
|---|---|---|---|---|---|---|---|---|---|---|
| ATOM | 940 | N | SER A | 513 | 16.947 | 37.963 | 75.424 | 1.00 | 21.26 | N |

| | | | | | | | | | | | |
|---|---|---|---|---|---|---|---|---|---|---|---|
| ANISOU | 940 | N | SER A | 513 | 3014 | 2102 | 2963 | 222 | −10 | 104 | N |

| | | | | | | | | | | |
|---|---|---|---|---|---|---|---|---|---|---|
| ATOM | 941 | CA | SER A | 513 | 17.808 | 38.469 | 76.418 | 1.00 | 22.17 | C |

| | | | | | | | | | | | |
|---|---|---|---|---|---|---|---|---|---|---|---|
| ANISOU | 941 | CA | SER A | 513 | 2947 | 2470 | 3006 | 102 | −75 | 116 | C |

| | | | | | | | | | | |
|---|---|---|---|---|---|---|---|---|---|---|
| ATOM | 942 | C | SER A | 513 | 18.251 | 37.509 | 77.507 | 1.00 | 19.87 | C |

| | | | | | | | | | | | |
|---|---|---|---|---|---|---|---|---|---|---|---|
| ANISOU | 942 | C | SER A | 513 | 2649 | 2135 | 2768 | −92 | 70 | 38 | C |

| | | | | | | | | | | |
|---|---|---|---|---|---|---|---|---|---|---|
| ATOM | 943 | O | SER A | 513 | 19.119 | 37.874 | 78.252 | 1.00 | 19.87 | O |

| | | | | | | | | | | | |
|---|---|---|---|---|---|---|---|---|---|---|---|
| ANISOU | 943 | O | SER A | 513 | 2798 | 1930 | 2822 | −10 | 80 | 291 | O |

| | | | | | | | | | | |
|---|---|---|---|---|---|---|---|---|---|---|
| ATOM | 944 | CB | SER A | 513 | 17.108 | 39.688 | 77.039 | 1.00 | 25.47 | C |

| | | | | | | | | | | | |
|---|---|---|---|---|---|---|---|---|---|---|---|
| ANISOU | 944 | CB | SER A | 513 | 3544 | 2743 | 3392 | 172 | 69 | −97 | C |

| | | | | | | | | | | |
|---|---|---|---|---|---|---|---|---|---|---|
| ATOM | 945 | OG | SER A | 513 | 16.001 | 39.285 | 77.795 | 1.00 | 27.69 | O |

| | | | | | | | | | | | |
|---|---|---|---|---|---|---|---|---|---|---|---|
| ANISOU | 945 | OG | SER A | 513 | 3642 | 3068 | 3810 | 241 | 217 | −215 | O |

| | | | | | | | | | | |
|---|---|---|---|---|---|---|---|---|---|---|
| ATOM | 946 | N | HIS A | 514 | 17.661 | 36.294 | 77.584 | 1.00 | 19.42 | N |

| | | | | | | | | | | | |
|---|---|---|---|---|---|---|---|---|---|---|---|
| ANISOU | 946 | N | HIS A | 514 | 2705 | 2009 | 2664 | 46 | 179 | 207 | N |

| | | | | | | | | | | |
|---|---|---|---|---|---|---|---|---|---|---|
| ATOM | 947 | CA | HIS A | 514 | 18.077 | 35.398 | 78.679 | 1.00 | 18.23 | C |

| | | | | | | | | | | | |
|---|---|---|---|---|---|---|---|---|---|---|---|
| ANISOU | 947 | CA | HIS A | 514 | 2600 | 1942 | 2384 | 76 | 155 | −16 | C |

| | | | | | | | | | | |
|---|---|---|---|---|---|---|---|---|---|---|
| ATOM | 948 | C | HIS A | 514 | 17.852 | 33.956 | 78.254 | 1.00 | 16.65 | C |

| | | | | | | | | | | | |
|---|---|---|---|---|---|---|---|---|---|---|---|
| ANISOU | 948 | C | HIS A | 514 | 2091 | 1960 | 2275 | 3 | 97 | −18 | C |

| | | | | | | | | | | |
|---|---|---|---|---|---|---|---|---|---|---|
| ATOM | 949 | O | HIS A | 514 | 17.070 | 33.726 | 77.331 | 1.00 | 18.87 | O |

| | | | | | | | | | | | |
|---|---|---|---|---|---|---|---|---|---|---|---|
| ANISOU | 949 | O | HIS A | 514 | 2639 | 2068 | 2462 | 176 | −225 | 164 | O |

| | | | | | | | | | | |
|---|---|---|---|---|---|---|---|---|---|---|
| ATOM | 950 | CB | HIS A | 514 | 17.171 | 35.673 | 79.900 | 1.00 | 18.92 | C |

| | | | | | | | | | | | |
|---|---|---|---|---|---|---|---|---|---|---|---|
| ANISOU | 950 | CB | HIS A | 514 | 2673 | 2202 | 2315 | 6 | 188 | 112 | C |

| | | | | | | | | | | |
|---|---|---|---|---|---|---|---|---|---|---|
| ATOM | 951 | CG | HIS A | 514 | 17.591 | 34.942 | 81.132 | 1.00 | 19.11 | C |

| | | | | | | | | | | | |
|---|---|---|---|---|---|---|---|---|---|---|---|
| ANISOU | 951 | CG | HIS A | 514 | 2472 | 2322 | 2468 | 89 | 136 | 70 | C |

| | | | | | | | | | | |
|---|---|---|---|---|---|---|---|---|---|---|
| ATOM | 952 | ND1 | HIS A | 514 | 18.865 | 34.979 | 81.688 | 1.00 | 19.64 | N |

| | | | | | | | | | | | |
|---|---|---|---|---|---|---|---|---|---|---|---|
| ANISON | 952 | ND1 | HIS A | 514 | 2736 | 2434 | 2293 | 169 | 110 | 164 | N |

| | | | | | | | | | | |
|---|---|---|---|---|---|---|---|---|---|---|
| ATOM | 953 | CD2 | HIS A | 514 | 16.809 | 34.198 | 81.942 | 1.00 | 19.33 | C |

| | | | | | | | | | | | |
|---|---|---|---|---|---|---|---|---|---|---|---|
| ANISOU | 953 | CD2 | HIS A | 514 | 2766 | 2312 | 2266 | 190 | 221 | −38 | C |

| | | | | | | | | | | |
|---|---|---|---|---|---|---|---|---|---|---|
| ATOM | 954 | CE1 | HIS A | 514 | 18.834 | 34.229 | 82.799 | 1.00 | 20.67 | C |

| | | | | | | | | | | | |
|---|---|---|---|---|---|---|---|---|---|---|---|
| ANISOU | 954 | CE1 | HIS A | 514 | 2913 | 2558 | 2382 | 116 | 204 | 127 | C |

| | | | | | | | | | | |
|---|---|---|---|---|---|---|---|---|---|---|
| ATOM | 955 | NE2 | HIS A | 514 | 17.611 | 33.760 | 82.956 | 1.00 | 18.73 | N |

| | | | | | | | | | | | |
|---|---|---|---|---|---|---|---|---|---|---|---|
| ANISOU | 955 | NE2 | HIS A | 514 | 2531 | 2269 | 2316 | 109 | 107 | −78 | N |

| | | | | | | | | | | |
|---|---|---|---|---|---|---|---|---|---|---|
| ATOM | 956 | N | TRP A | 515 | 18.625 | 33.043 | 78.840 | 1.00 | 15.92 | N |

| | | | | | | | | | | | |
|---|---|---|---|---|---|---|---|---|---|---|---|
| ANISOU | 956 | N | TRP A | 515 | 2444 | 1538 | 2069 | −36 | 196 | −66 | N |

| | | | | | | | | | | |
|---|---|---|---|---|---|---|---|---|---|---|
| ATOM | 957 | CA | TRP A | 515 | 18.452 | 31.617 | 78.550 | 1.00 | 15.22 | C |

| | | | | | | | | | | | |
|---|---|---|---|---|---|---|---|---|---|---|---|
| ANISOU | 957 | CA | TRP A | 515 | 2159 | 1643 | 1980 | −20 | 208 | −57 | C |

| | | | | | | | | | | |
|---|---|---|---|---|---|---|---|---|---|---|
| ATOM | 958 | C | TRP A | 515 | 18.080 | 30.936 | 79.876 | 1.00 | 14.54 | C |

| | | | | | | | | | | | |
|---|---|---|---|---|---|---|---|---|---|---|---|
| ANISOU | 958 | C | TRP A | 515 | 1759 | 1713 | 2052 | 33 | 265 | −57 | C |

| | | | | | | | | | | |
|---|---|---|---|---|---|---|---|---|---|---|
| ATOM | 959 | O | TRP A | 515 | 18.694 | 31.235 | 80.873 | 1.00 | 15.86 | O |

| | | | | | | | | | | | |
|---|---|---|---|---|---|---|---|---|---|---|---|
| ANISOU | 959 | O | TRP A | 515 | 2185 | 1588 | 2253 | 75 | 166 | 11 | O |

| | | | | | | | | | | |
|---|---|---|---|---|---|---|---|---|---|---|
| ATOM | 960 | CB | TRP A | 515 | 19.824 | 31.063 | 78.074 | 1.00 | 15.42 | C |

| | | | | | | | | | | | |
|---|---|---|---|---|---|---|---|---|---|---|---|
| ANISOU | 960 | CB | TRP A | 515 | 2021 | 1862 | 1978 | 77 | 91 | 34 | C |

| | | | | | | | | | | |
|---|---|---|---|---|---|---|---|---|---|---|
| ATOM | 961 | CG | TRP A | 515 | 20.283 | 31.451 | 76.692 | 1.00 | 15.37 | C |

| | | | | | | | | | | | |
|---|---|---|---|---|---|---|---|---|---|---|---|
| ANISOU | 961 | CG | TRP A | 515 | 1902 | 1948 | 1991 | 126 | −47 | 153 | C |

| | | | | | | | | | | |
|---|---|---|---|---|---|---|---|---|---|---|
| ATOM | 962 | CD1 | TRP A | 515 | 21.363 | 32.251 | 76.387 | 1.00 | 15.39 | C |

| | | | | | | | | | | | |
|---|---|---|---|---|---|---|---|---|---|---|---|
| ANISON | 962 | CD1 | TRP A | 515 | 2241 | 1728 | 1878 | 61 | −1 | 232 | C |

| | | | | | | | | | | |
|---|---|---|---|---|---|---|---|---|---|---|
| ATOM | 963 | CD2 | TRP A | 515 | 19.731 | 31.037 | 75.433 | 1.00 | 14.53 | C |

| | | | | | | | | | | | |
|---|---|---|---|---|---|---|---|---|---|---|---|
| ANISOU | 963 | CD2 | TRP A | 515 | 1884 | 1593 | 2045 | 224 | 54 | −11 | C |

| | | | | | | | | | | |
|---|---|---|---|---|---|---|---|---|---|---|
| ATOM | 964 | NE1 | TRP A | 515 | 21.505 | 32.333 | 75.026 | 1.00 | 15.87 | N |

| | | | | | | | | | | | |
|---|---|---|---|---|---|---|---|---|---|---|---|
| ANISOU | 964 | NH1 | TRP A | 515 | 2067 | 2032 | 1932 | −107 | −121 | 94 | N |

| | | | | | | | | | | |
|---|---|---|---|---|---|---|---|---|---|---|
| ATOM | 965 | CE2 | TRP A | 515 | 20.501 | 31.604 | 74.419 | 1.00 | 15.72 | C |

| | | | | | | | | | | | |
|---|---|---|---|---|---|---|---|---|---|---|---|
| ANISOU | 965 | CE2 | TRP A | 515 | 2216 | 1651 | 2106 | 64 | −41 | 1 | C |

| | | | | | | | | | | |
|---|---|---|---|---|---|---|---|---|---|---|
| ATOM | 966 | CE3 | TRP A | 515 | 18.659 | 30.194 | 75.094 | 1.00 | 16.46 | C |

| | | | | | | | | | | | |
|---|---|---|---|---|---|---|---|---|---|---|---|
| ANISOU | 966 | CE3 | TRP A | 515 | 2252 | 1816 | 2186 | 94 | −211 | −40 | C |

| | | | | | | | | | | |
|---|---|---|---|---|---|---|---|---|---|---|
| ATOM | 967 | CZ2 | TRP A | 515 | 20.261 | 31.407 | 73.064 | 1.00 | 16.54 | C |

| | | | | | | | | | | | |
|---|---|---|---|---|---|---|---|---|---|---|---|
| ANISOU | 967 | CZ2 | TRP A | 515 | 2189 | 1984 | 2110 | 61 | 45 | 76 | C |

| | | | | | | | | | | |
|---|---|---|---|---|---|---|---|---|---|---|
| ATOM | 968 | CZ3 | TRP A | 515 | 18.420 | 29.985 | 73.742 | 1.00 | 17.08 | C |

| | | | | | | | | | | | |
|---|---|---|---|---|---|---|---|---|---|---|---|
| ANISOU | 968 | CZ3 | TRP A | 515 | 2443 | 2024 | 2021 | 144 | 28 | 114 | C |

| | | | | | | | | | | |
|---|---|---|---|---|---|---|---|---|---|---|
| ATOM | 969 | CH2 | TRP A | 515 | 19.202 | 30.604 | 72.747 | 1.00 | 16.82 | C |

| | | | | | | | | | | | |
|---|---|---|---|---|---|---|---|---|---|---|---|
| ANISOU | 969 | CH2 | TRP A | 515 | 2107 | 2095 | 2188 | 94 | 66 | 19 | C |

| | | | | | | | | | | |
|---|---|---|---|---|---|---|---|---|---|---|
| ATOM | 970 | N | ALA A | 516 | 17.177 | 29.933 | 79.790 | 1.00 | 15 | N |

| | | | | | | | | | | | |
|---|---|---|---|---|---|---|---|---|---|---|---|
| ANISOU | 970 | N | ALA A | 516 | 2188 | 1663 | 2100 | −43 | 248 | 88 | N |

| | | | | | | | | | | |
|---|---|---|---|---|---|---|---|---|---|---|
| ATOM | 971 | CA | ALA A | 516 | 16.818 | 29.128 | 80.964 | 1.00 | 15.16 | C |

| | | | | | | | | | | | |
|---|---|---|---|---|---|---|---|---|---|---|---|
| ANISOU | 971 | CA | ALA A | 516 | 1918 | 1825 | 2019 | −47 | 221 | −70 | C |

| | | | | | | | | | | |
|---|---|---|---|---|---|---|---|---|---|---|
| ATOM | 972 | C | ALA A | 516 | 16.616 | 27.692 | 80.445 | 1.00 | 15.33 | C |

| | | | | | | | | | | | |
|---|---|---|---|---|---|---|---|---|---|---|---|
| ANISOU | 972 | C | ALA A | 516 | 1861 | 1857 | 2107 | −135 | 87 | −64 | C |

| | | | | | | | | | | |
|---|---|---|---|---|---|---|---|---|---|---|
| ATOM | 973 | O | ALA A | 516 | 16.833 | 27.425 | 79.244 | 1.00 | 14.82 | O |

| | | | | | | | | | | | |
|---|---|---|---|---|---|---|---|---|---|---|---|
| ANISOU | 973 | O | ALA A | 516 | 1910 | 1704 | 2018 | −83 | 53 | 67 | O |

| | | | | | | | | | | |
|---|---|---|---|---|---|---|---|---|---|---|
| ATOM | 974 | CB | ALA A | 516 | 15.612 | 29.648 | 81.735 | 1.00 | 16.96 | C |

| | | | | | | | | | | | |
|---|---|---|---|---|---|---|---|---|---|---|---|
| ANISOU | 974 | CB | ALA A | 516 | 2327 | 1740 | 2378 | 30 | 354 | −70 | C |

| | | | | | | | | | | |
|---|---|---|---|---|---|---|---|---|---|---|
| ATOM | 975 | N | LEU A | 517 | 16.205 | 26.789 | 81.303 | 1.00 | 17.24 | N |

| | | | | | | | | | | | |
|---|---|---|---|---|---|---|---|---|---|---|---|
| ANISOU | 975 | N | LEU A | 517 | 2556 | 1839 | 2157 | −63 | 38 | −68 | N |

| | | | | | | | | | | |
|---|---|---|---|---|---|---|---|---|---|---|
| ATOM | 976 | CA | LEU A | 517 | 16.102 | 25.382 | 80.859 | 1.00 | 16.67 | C |

| | | | | | | | | | | | |
|---|---|---|---|---|---|---|---|---|---|---|---|
| ANISOU | 976 | CA | LEU A | 517 | 2261 | 1740 | 2331 | −205 | 115 | −18 | C |

| | | | | | | | | | | |
|---|---|---|---|---|---|---|---|---|---|---|
| ATOM | 977 | C | LEU A | 517 | 14.928 | 24.654 | 81.483 | 1.00 | 16.87 | C |

| | | | | | | | | | | | |
|---|---|---|---|---|---|---|---|---|---|---|---|
| ANISOU | 977 | C | LEU A | 517 | 2301 | 1702 | 2408 | −126 | 214 | 2 | C |

| | | | | | | | | | | |
|---|---|---|---|---|---|---|---|---|---|---|
| ATOM | 978 | O | LEU A | 517 | 14.633 | 24.806 | 82.660 | 1.00 | 17.92 | O |

| | | | | | | | | | | | |
|---|---|---|---|---|---|---|---|---|---|---|---|
| ANISOU | 978 | O | LEU A | 517 | 2620 | 1695 | 2493 | −226 | 46 | −47 | O |

| | | | | | | | | | | |
|---|---|---|---|---|---|---|---|---|---|---|
| ATOM | 979 | CB | LEU A | 517 | 17.380 | 24.700 | 81.401 | 1.00 | 17.54 | C |

| | | | | | | | | | | | |
|---|---|---|---|---|---|---|---|---|---|---|---|
| ANISOU | 979 | CB | LEU A | 517 | 2187 | 1807 | 2670 | −79 | 140 | −39 | C |

| | | | | | | | | | | |
|---|---|---|---|---|---|---|---|---|---|---|
| ATOM | 980 | CG | LEU A | 517 | 17.538 | 23.199 | 81.135 | 1.00 | 16.59 | C |

| | | | | | | | | | | | |
|---|---|---|---|---|---|---|---|---|---|---|---|
| ANISOU | 980 | CG | LEU A | 517 | 2074 | 1810 | 2420 | −31 | 92 | −35 | C |

| | | | | | | | | | | |
|---|---|---|---|---|---|---|---|---|---|---|
| ATOM | 981 | CD1 | LEU A | 517 | 17.695 | 22.984 | 79.632 | 1.00 | 18.96 | C |

| | | | | | | | | | | | |
|---|---|---|---|---|---|---|---|---|---|---|---|
| ANISOU | 981 | CD1 | LEU A | 517 | 2741 | 2017 | 2445 | 104 | 33 | −124 | C |

| | | | | | | | | | | |
|---|---|---|---|---|---|---|---|---|---|---|
| ATOM | 982 | CD2 | LEU A | 517 | 18.763 | 22.599 | 81.870 | 1.00 | 16.79 | C |

| | | | | | | | | | | | |
|---|---|---|---|---|---|---|---|---|---|---|---|
| ANISOU | 982 | CD2 | LEU A | 517 | 2199 | 1852 | 2331 | 49 | 76 | −57 | C |

| | | | | | | | | | | |
|---|---|---|---|---|---|---|---|---|---|---|
| ATOM | 983 | N | GLY A | 518 | 14.300 | 23.846 | 80.615 | 1.00 | 16.34 | N |

| | | | | | | | | | | | |
|---|---|---|---|---|---|---|---|---|---|---|---|
| ANISOU | 983 | N | GLY A | 518 | 1852 | 1842 | 2514 | −188 | 65 | 28 | N |

| | | | | | | | | | | |
|---|---|---|---|---|---|---|---|---|---|---|
| ATOM | 984 | CA | GLY A | 518 | 13.259 | 22.949 | 81.118 | 1.00 | 17.73 | C |

| | | | | | | | | | | | |
|---|---|---|---|---|---|---|---|---|---|---|---|
| ANISOU | 984 | CA | GLY A | 518 | 2289 | 1935 | 2511 | −197 | 278 | 12 | C |

| | | | | | | | | | | |
|---|---|---|---|---|---|---|---|---|---|---|
| ATOM | 985 | C | GLY A | 518 | 13.857 | 21.543 | 81.157 | 1.00 | 18.49 | C |

| | | | | | | | | | | | |
|---|---|---|---|---|---|---|---|---|---|---|---|
| ANISOU | 985 | C | GLY A | 518 | 2443 | 2060 | 2523 | −97 | 170 | 45 | C |

| | | | | | | | | | | |
|---|---|---|---|---|---|---|---|---|---|---|
| ATOM | 986 | O | GLY A | 518 | 14.626 | 21.177 | 80.266 | 1.00 | 16.84 | O |

| | | | | | | | | | | | |
|---|---|---|---|---|---|---|---|---|---|---|---|
| ANISOU | 986 | O | GLY A | 518 | 2257 | 1525 | 2616 | −236 | 156 | −18 | O |

| | | | | | | | | | | |
|---|---|---|---|---|---|---|---|---|---|---|
| ATOM | 987 | N | ILE A | 519 | 13.489 | 20.784 | 82.213 | 1.00 | 18.37 | N |

| | | | | | | | | | | | |
|---|---|---|---|---|---|---|---|---|---|---|---|
| ANISOU | 987 | N | ILE A | 519 | 2462 | 2085 | 2432 | −64 | 312 | 28 | N |

| | | | | | | | | | | |
|---|---|---|---|---|---|---|---|---|---|---|
| ATOM | 988 | CA | ILE A | 519 | 13.961 | 19.393 | 82.287 | 1.00 | 18.08 | C |

| | | | | | | | | | | | |
|---|---|---|---|---|---|---|---|---|---|---|---|
| ANISOU | 988 | CA | ILE A | 519 | 2190 | 2084 | 2596 | −166 | 65 | 106 | C |

| | | | | | | | | | | |
|---|---|---|---|---|---|---|---|---|---|---|
| ATOM | 989 | C | ILE A | 519 | 12.721 | 18.496 | 82.461 | 1.00 | 18.84 | C |

| | | | | | | | | | | | |
|---|---|---|---|---|---|---|---|---|---|---|---|
| ANISOU | 989 | C | ILE A | 519 | 2477 | 2050 | 2632 | −218 | 178 | 68 | C |

| | | | | | | | | | | |
|---|---|---|---|---|---|---|---|---|---|---|
| ATOM | 990 | O | ILE A | 519 | 12.097 | 18.576 | 83.513 | 1.00 | 18.98 | O |

| | | | | | | | | | | | |
|---|---|---|---|---|---|---|---|---|---|---|---|
| ANISOU | 990 | O | ILE A | 519 | 2520 | 2015 | 2675 | −390 | 236 | 113 | O |

| | | | | | | | | | | |
|---|---|---|---|---|---|---|---|---|---|---|
| ATOM | 991 | CB | ILE A | 519 | 14.928 | 19.165 | 83.434 | 1.00 | 18.19 | C |

| | | | | | | | | | | | |
|---|---|---|---|---|---|---|---|---|---|---|---|
| ANISOU | 991 | CB | ILE A | 519 | 2571 | 1902 | 2437 | −31 | 65 | 277 | C |

| | | | | | | | | | | |
|---|---|---|---|---|---|---|---|---|---|---|
| ATOM | 992 | CG1 | ILE A | 519 | 16.160 | 20.087 | 83.379 | 1.00 | 18.07 | C |

| | | | | | | | | | | | |
|---|---|---|---|---|---|---|---|---|---|---|---|
| ANISOU | 992 | CG1 | ILE A | 519 | 2194 | 2251 | 2421 | −33 | −20 | 117 | C |

| | | | | | | | | | | |
|---|---|---|---|---|---|---|---|---|---|---|
| ATOM | 993 | CG2 | ILE A | 519 | 15.457 | 17.705 | 83.457 | 1.00 | 18.79 | C |

| | | | | | | | | | | | |
|---|---|---|---|---|---|---|---|---|---|---|---|
| ANISOU | 993 | CG2 | ILE A | 519 | 2457 | 1978 | 2706 | 89 | 32 | 147 | C |

| | | | | | | | | | | |
|---|---|---|---|---|---|---|---|---|---|---|
| ATOM | 994 | CD1 | ILE A | 519 | 17.203 | 19.991 | 84.485 | 1.00 | 19.76 | C |

| | | | | | | | | | | | |
|---|---|---|---|---|---|---|---|---|---|---|---|
| ANISOU | 994 | CD1 | ILE A | 519 | 2621 | 2656 | 2233 | −209 | 29 | 161 | C |

| | | | | | | | | | | |
|---|---|---|---|---|---|---|---|---|---|---|
| ATOM | 995 | N | ILE A | 520 | 12.411 | 17.704 | 81.455 | 1.00 | 18.78 | N |

| | | | | | | | | | | | |
|---|---|---|---|---|---|---|---|---|---|---|---|
| ANISOU | 995 | N | ILE A | 520 | 2245 | 2275 | 2616 | −395 | 131 | 158 | N |

| | | | | | | | | | | |
|---|---|---|---|---|---|---|---|---|---|---|
| ATOM | 996 | CA | ILE A | 520 | 11.299 | 16.725 | 81.676 | 1.00 | 19.96 | C |

| | | | | | | | | | | | |
|---|---|---|---|---|---|---|---|---|---|---|---|
| ANISOU | 996 | CA | ILE A | 520 | 2445 | 2272 | 2866 | −295 | 185 | 77 | C |

| | | | | | | | | | | |
|---|---|---|---|---|---|---|---|---|---|---|
| ATOM | 997 | C | ILE A | 520 | 11.989 | 15.440 | 82.129 | 1.00 | 20.99 | C |

| | | | | | | | | | | | |
|---|---|---|---|---|---|---|---|---|---|---|---|
| ANISOU | 997 | C | ILE A | 520 | 2539 | 2467 | 2968 | −177 | 92 | 40 | C |

| | | | | | | | | | | |
|---|---|---|---|---|---|---|---|---|---|---|
| ATOM | 998 | O | ILE A | 520 | 12.770 | 14.877 | 81.375 | 1.00 | 20.55 | O |

| | | | | | | | | | | | |
|---|---|---|---|---|---|---|---|---|---|---|---|
| ANISOU | 998 | O | ILE A | 520 | 3031 | 1906 | 2869 | −193 | 197 | −63 | O |

| | | | | | | | | | | |
|---|---|---|---|---|---|---|---|---|---|---|
| ATOM | 999 | CB | ILE A | 520 | 10.583 | 16.508 | 80.366 | 1.00 | 19.40 | C |

| | | | | | | | | | | | |
|---|---|---|---|---|---|---|---|---|---|---|---|
| ANISOU | 999 | CB | ILE A | 520 | 2298 | 2316 | 2757 | −260 | 177 | 29 | C |

| | | | | | | | | | | |
|---|---|---|---|---|---|---|---|---|---|---|
| ATOM | 1000 | CG1 | ILE A | 520 | 9.862 | 17.790 | 79.936 | 1.00 | 21.11 | C |

| | | | | | | | | | | | |
|---|---|---|---|---|---|---|---|---|---|---|---|
| ANISOU | 1000 | CG1 | ILE A | 520 | 2590 | 2625 | 2806 | 12 | 95 | 23 | C |

| | | | | | | | | | | |
|---|---|---|---|---|---|---|---|---|---|---|
| ATOM | 1001 | CG2 | ILE A | 520 | 9.585 | 15.324 | 80.398 | 1.00 | 20.96 | C |

| | | | | | | | | | | | |
|---|---|---|---|---|---|---|---|---|---|---|---|
| ANISOU | 1001 | CG2 | ILE A | 520 | 2654 | 2379 | 2933 | −392 | 102 | −83 | C |

| | | | | | | | | | | |
|---|---|---|---|---|---|---|---|---|---|---|
| ATOM | 1002 | CD1 | ILE A | 520 | 9.506 | 17.824 | 78.463 | 1.00 | 22.20 | C |

| | | | | | | | | | | | |
|---|---|---|---|---|---|---|---|---|---|---|---|
| ANISOU | 1002 | CD1 | ILE A | 520 | 2753 | 2912 | 2769 | −36 | 130 | 99 | C |

| | | | | | | | | | | |
|---|---|---|---|---|---|---|---|---|---|---|
| ATOM | 1003 | N | ASP A | 521 | 11.704 | 14.978 | 83.334 | 1.00 | 23.09 | N |

| | | | | | | | | | | | |
|---|---|---|---|---|---|---|---|---|---|---|---|
| ANISOU | 1003 | N | ASP A | 521 | 3058 | 2744 | 2971 | −180 | 38 | 13 | N |

| | | | | | | | | | | |
|---|---|---|---|---|---|---|---|---|---|---|
| ATOM | 1004 | CA | ASP A | 521 | 12.301 | 13.744 | 83.832 | 1.00 | 23.20 | C |

| | | | | | | | | | | | |
|---|---|---|---|---|---|---|---|---|---|---|---|
| ANISOU | 1004 | CA | ASP A | 521 | 2777 | 2744 | 3294 | −218 | 31 | 81 | C |

| | | | | | | | | | | |
|---|---|---|---|---|---|---|---|---|---|---|
| ATOM | 1005 | C | ASP A | 521 | 11.200 | 12.666 | 83.801 | 1.00 | 23.64 | C |

| | | | | | | | | | | | |
|---|---|---|---|---|---|---|---|---|---|---|---|
| ANISOU | 1005 | C | ASP A | 521 | 3012 | 2765 | 3204 | −287 | −55 | 29 | C |

| | | | | | | | | | | |
|---|---|---|---|---|---|---|---|---|---|---|
| ATOM | 1006 | O | ASP A | 521 | 10.411 | 12.644 | 84.734 | 1.00 | 22.75 | O |

| | | | | | | | | | | | |
|---|---|---|---|---|---|---|---|---|---|---|---|
| ANISOU | 1006 | O | ASP A | 521 | 2999 | 2369 | 3276 | −408 | 8 | −135 | O |

| | | | | | | | | | | |
|---|---|---|---|---|---|---|---|---|---|---|
| ATOM | 1007 | CB | ASP A | 521 | 12.816 | 13.930 | 85.247 | 1.00 | 25.50 | C |

| | | | | | | | | | | | |
|---|---|---|---|---|---|---|---|---|---|---|---|
| ANISOU | 1007 | CB | ASP A | 521 | 3215 | 3212 | 3263 | −161 | 60 | 42 | C |

| | | | | | | | | | | |
|---|---|---|---|---|---|---|---|---|---|---|
| ATOM | 1008 | CG | ASP A | 521 | 13.805 | 12.842 | 85.638 | 1.00 | 27.77 | C |

| | | | | | | | | | | | |
|---|---|---|---|---|---|---|---|---|---|---|---|
| ANISOU | 1008 | CG | ASP A | 521 | 3326 | 3511 | 3714 | 48 | 28 | 96 | C |

| | | | | | | | | | | |
|---|---|---|---|---|---|---|---|---|---|---|
| ATOM | 1009 | OD1 | ASP A | 521 | 13.532 | 11.677 | 85.267 | 1.00 | 28.61 | O |

| | | | | | | | | | | | |
|---|---|---|---|---|---|---|---|---|---|---|---|
| ANISOU | 1009 | OD1 | ASP A | 521 | 3384 | 3609 | 3878 | −30 | −11 | 78 | O |

| | | | | | | | | | | |
|---|---|---|---|---|---|---|---|---|---|---|
| ATOM | 1010 | OD2 | ASP A | 521 | 14.841 | 13.167 | 86.293 | 1.00 | 30.97 | O |

| | | | | | | | | | | | |
|---|---|---|---|---|---|---|---|---|---|---|---|
| ANISOU | 1010 | OD2 | ASP A | 521 | 3687 | 4027 | 4054 | −123 | −37 | 30 | O |

| | | | | | | | | | | |
|---|---|---|---|---|---|---|---|---|---|---|
| ATOM | 1011 | N | LEU A | 522 | 11.239 | 11.868 | 82.746 | 1.00 | 24.20 | N |

| | | | | | | | | | | | |
|---|---|---|---|---|---|---|---|---|---|---|---|
| ANISOU | 1011 | N | LEU A | 522 | 3116 | 2732 | 3348 | −304 | 127 | 26 | N |

| | | | | | | | | | | |
|---|---|---|---|---|---|---|---|---|---|---|
| ATOM | 1012 | CA | LEU A | 522 | 10.199 | 10.834 | 82.599 | 1.00 | 25.59 | C |

| | | | | | | | | | | | |
|---|---|---|---|---|---|---|---|---|---|---|---|
| ANISOU | 1012 | CA | LEU A | 522 | 3039 | 3104 | 3579 | −334 | −29 | 46 | C |

| | | | | | | | | | | |
|---|---|---|---|---|---|---|---|---|---|---|
| ATOM | 1013 | C | LEU A | 522 | 10.306 | 9.771 | 83.657 | 1.00 | 27.15 | C |

| | | | | | | | | | | | |
|---|---|---|---|---|---|---|---|---|---|---|---|
| ANISOU | 1013 | C | LEU A | 522 | 3284 | 3354 | 3677 | −85 | 58 | 163 | C |

| | | | | | | | | | | |
|---|---|---|---|---|---|---|---|---|---|---|
| ATOM | 1014 | O | LEU A | 522 | 9.276 | 9.216 | 84.106 | 1.00 | 27.20 | O |

| | | | | | | | | | | | |
|---|---|---|---|---|---|---|---|---|---|---|---|
| ANISOU | 1014 | O | LEU A | 522 | 3334 | 3290 | 3711 | −238 | −74 | 258 | O |

| | | | | | | | | | | |
|---|---|---|---|---|---|---|---|---|---|---|
| ATOM | 1015 | CB | LEU A | 522 | 10.309 | 10.306 | 81.137 | 1.00 | 26.24 | C |

| | | | | | | | | | | | |
|---|---|---|---|---|---|---|---|---|---|---|---|
| ANISOU | 1015 | CB | LEU A | 522 | 3236 | 3094 | 3640 | −249 | 56 | −64 | C |

| | | | | | | | | | | |
|---|---|---|---|---|---|---|---|---|---|---|
| ATOM | 1016 | CG | LEU A | 522 | 10.044 | 11.460 | 80.162 | 1.00 | 29.23 | C |

| | | | | | | | | | | | |
|---|---|---|---|---|---|---|---|---|---|---|---|
| ANISOU | 1016 | CG | LEU A | 522 | 3716 | 3702 | 3687 | 105 | −2 | 91 | C |

| | | | | | | | | | | |
|---|---|---|---|---|---|---|---|---|---|---|
| ATOM | 1017 | CD1 | LEU A | 522 | 10.999 | 11.499 | 78.980 | 1.00 | 28.63 | C |

| | | | | | | | | | | | |
|---|---|---|---|---|---|---|---|---|---|---|---|
| ANISOU | 1017 | CD1 | LEU A | 522 | 3741 | 3414 | 3724 | 56 | 72 | 122 | C |

| | | | | | | | | | | |
|---|---|---|---|---|---|---|---|---|---|---|
| ATOM | 1018 | CD2 | LEU A | 522 | 8.578 | 11.590 | 79.786 | 1.00 | 28.59 | C |

| | | | | | | | | | | | |
|---|---|---|---|---|---|---|---|---|---|---|---|
| ANISOU | 1018 | CD2 | LEU A | 522 | 3622 | 3424 | 3818 | −180 | 26 | 35 | C |

| | | | | | | | | | | |
|---|---|---|---|---|---|---|---|---|---|---|
| ATOM | 1019 | N | LYS A | 523 | 11.518 | 9.444 | 84.086 | 1.00 | 29.20 | N |

| | | | | | | | | | | | |
|---|---|---|---|---|---|---|---|---|---|---|---|
| ANISOU | 1019 | N | LYS A | 523 | 3423 | 3708 | 3964 | −255 | −71 | 154 | N |

| | | | | | | | | | | |
|---|---|---|---|---|---|---|---|---|---|---|
| ATOM | 1020 | CA | LYS A | 523 | 11.695 | 8.410 | 85.115 | 1.00 | 31.44 | C |

| | | | | | | | | | | | |
|---|---|---|---|---|---|---|---|---|---|---|---|
| ANISOU | 1020 | CA | LYS A | 523 | 4022 | 3843 | 4080 | 6 | −98 | 154 | C |

| | | | | | | | | | | |
|---|---|---|---|---|---|---|---|---|---|---|
| ATOM | 1021 | C | LYS A | 523 | 11.222 | 8.884 | 86.469 | 1.00 | 30.80 | C |

| | | | | | | | | | | | |
|---|---|---|---|---|---|---|---|---|---|---|---|
| ANISOU | 1021 | C | LYS A | 523 | 3802 | 3719 | 4183 | 7 | −3 | 128 | C |

| | | | | | | | | | | |
|---|---|---|---|---|---|---|---|---|---|---|
| ATOM | 1022 | O | LYS A | 523 | 10.569 | 8.154 | 87.225 | 1.00 | 31.37 | O |

| | | | | | | | | | | | |
|---|---|---|---|---|---|---|---|---|---|---|---|
| ANISOU | 1022 | O | LYS A | 523 | 3915 | 3689 | 4314 | −42 | −65 | 297 | O |

| | | | | | | | | | | |
|---|---|---|---|---|---|---|---|---|---|---|
| ATOM | 1023 | CB | LYS A | 523 | 13.17S | 8.019 | 85.128 | 1.00 | 34.12 | C |

| | | | | | | | | | | | |
|---|---|---|---|---|---|---|---|---|---|---|---|
| ANISOU | 1023 | CB | LYS A | 523 | 4074 | 4323 | 4568 | −45 | 54 | 5 | C |

| | | | | | | | | | | |
|---|---|---|---|---|---|---|---|---|---|---|
| ATOM | 1024 | CG | LYS A | 523 | 13.656 | 7.079 | 86.201 | 1.00 | 38.10 | C |

| | | | | | | | | | | | |
|---|---|---|---|---|---|---|---|---|---|---|---|
| ANISOU | 1024 | CG | LYS A | 523 | 4961 | 4694 | 4822 | 99 | −15 | 141 | C |

| | | | | | | | | | | |
|---|---|---|---|---|---|---|---|---|---|---|
| ATOM | 1025 | CD | LYS A | 523 | 15.127 | 7.308 | 86.553 | 1.00 | 41.94 | C |

| | | | | | | | | | | | |
|---|---|---|---|---|---|---|---|---|---|---|---|
| ANISOU | 1025 | CD | LYS A | 523 | 5187 | 5312 | 5435 | 1 | −91 | 16 | C |

| | | | | | | | | | | |
|---|---|---|---|---|---|---|---|---|---|---|
| ATOM | 1026 | CE | LYS A | 523 | 16.040 | 7.405 | 85.343 | 1.00 | 44.32 | C |

| | | | | | | | | | | | |
|---|---|---|---|---|---|---|---|---|---|---|---|
| ANISOU | 1026 | CE | LYS A | 523 | 5651 | 5660 | 5529 | 60 | 95 | 55 | C |

| | | | | | | | | | | |
|---|---|---|---|---|---|---|---|---|---|---|
| ATOM | 1027 | NZ | LYS A | 523 | 17.378 | 7.975 | 85.628 | 1.00 | 46.06 | N |

| | | | | | | | | | | | |
|---|---|---|---|---|---|---|---|---|---|---|---|
| ANISOU | 1027 | NZ | LYS A | 523 | 5831 | 5913 | 5756 | −60 | −93 | 21 | N |

| | | | | | | | | | | |
|---|---|---|---|---|---|---|---|---|---|---|
| ATOM | 1028 | N | LYS A | 524 | 11.497 | 10.140 | 86.836 | 1.00 | 29.18 | N |

| | | | | | | | | | | | |
|---|---|---|---|---|---|---|---|---|---|---|---|
| ANISOU | 1028 | N | LYS A | 524 | 3695 | 3597 | 3795 | −13 | −75 | 142 | N |

| | | | | | | | | | | |
|---|---|---|---|---|---|---|---|---|---|---|
| ATOM | 1029 | CA | LYS A | 524 | 11.062 | 10.677 | 88.120 | 1.00 | 28.80 | C |

| | | | | | | | | | | | |
|---|---|---|---|---|---|---|---|---|---|---|---|
| ANISOU | 1029 | CA | LYS A | 524 | 3580 | 3516 | 3846 | 66 | −20 | 123 | C |

| | | | | | | | | | | |
|---|---|---|---|---|---|---|---|---|---|---|
| ATOM | 1030 | C | LYS A | 524 | 9.664 | 11.283 | 88.059 | 1.00 | 26.40 | C |

| | | | | | | | | | | | |
|---|---|---|---|---|---|---|---|---|---|---|---|
| ANISOU | 1030 | C | LYS A | 524 | 3340 | 3146 | 3544 | −159 | 24 | 69 | C |

| | | | | | | | | | | |
|---|---|---|---|---|---|---|---|---|---|---|
| ATOM | 1031 | O | LYS A | 524 | 9.092 | 11.648 | 89.103 | 1.00 | 27.09 | O |

| | | | | | | | | | | | |
|---|---|---|---|---|---|---|---|---|---|---|---|
| ANISOU | 1031 | O | LYS A | 524 | 3377 | 3269 | 3647 | −24 | 35 | 106 | O |

| | | | | | | | | | | |
|---|---|---|---|---|---|---|---|---|---|---|
| ATOM | 1032 | CB | LYS A | 524 | 12.032 | 11.744 | 88.654 | 1.00 | 29.66 | C |

| | | | | | | | | | | | |
|---|---|---|---|---|---|---|---|---|---|---|---|
| ANISOU | 1032 | CB | LYS A | 524 | 3768 | 3683 | 3818 | −69 | 10 | 39 | C |

| | | | | | | | | | | |
|---|---|---|---|---|---|---|---|---|---|---|
| ATOM | 1033 | CG | LYS A | 524 | 13.466 | 11.227 | 88.794 | 1.00 | 32.19 | C |

| | | | | | | | | | | | |
|---|---|---|---|---|---|---|---|---|---|---|---|
| ANISOU | 1033 | CG | LYS A | 524 | 3842 | 4190 | 4197 | 50 | −12 | 56 | C |

| | | | | | | | | | | |
|---|---|---|---|---|---|---|---|---|---|---|
| ATOM | 1034 | CD | LYS A | 524 | 14.444 | 12.342 | 89.164 | 1.00 | 34.05 | C |

| | | | | | | | | | | | |
|---|---|---|---|---|---|---|---|---|---|---|---|
| ANISOU | 1034 | CD | LYS A | 524 | 4289 | 4315 | 4334 | −87 | 1 | −29 | C |

| | | | | | | | | | | |
|---|---|---|---|---|---|---|---|---|---|---|
| ATOM | 1035 | CE | LYS A | 524 | 15.909 | 12.024 | 89.102 | 1.00 | 34.85 | C |

| | | | | | | | | | | | |
|---|---|---|---|---|---|---|---|---|---|---|---|
| ANISOU | 1035 | CE | LYS A | 524 | 4338 | 4517 | 4389 | −39 | −25 | −51 | C |

| | | | | | | | | | | |
|---|---|---|---|---|---|---|---|---|---|---|
| ATOM | 1036 | NZ | LYS A | 524 | 16.514 | 11.646 | 87.793 | 1.00 | 34.69 | N |

| | | | | | | | | | | | |
|---|---|---|---|---|---|---|---|---|---|---|---|
| ANISOU | 1036 | NZ | LYS A | 524 | 4213 | 4629 | 4338 | −165 | 53 | 97 | N |

| | | | | | | | | | | |
|---|---|---|---|---|---|---|---|---|---|---|
| ATOM | 1037 | N | LYS A | 525 | 9.092 | 11.343 | 86.867 | 1.00 | 24.54 | N |

| | | | | | | | | | | | |
|---|---|---|---|---|---|---|---|---|---|---|---|
| ANISOU | 1037 | N | LYS A | 525 | 3232 | 2630 | 3462 | −213 | 127 | 220 | N |

| | | | | | | | | | | |
|---|---|---|---|---|---|---|---|---|---|---|
| ATOM | 1038 | CA | LYS A | 525 | 7.767 | 11.927 | 86.682 | 1.00 | 25.62 | C |

| | | | | | | | | | | | |
|---|---|---|---|---|---|---|---|---|---|---|---|
| ANISOU | 1038 | CA | LYS A | 525 | 3131 | 3067 | 3538 | −250 | 214 | 146 | C |

| | | | | | | | | | | |
|---|---|---|---|---|---|---|---|---|---|---|
| ATOM | 1039 | C | LYS A | 525 | 7.744 | 13.356 | 87.188 | 1.00 | 26.16 | C |

| | | | | | | | | | | | |
|---|---|---|---|---|---|---|---|---|---|---|---|
| ANISOU | 1039 | C | LYS A | 525 | 3279 | 3153 | 3508 | −149 | 284 | 59 | C |

| | | | | | | | | | | |
|---|---|---|---|---|---|---|---|---|---|---|
| ATOM | 1040 | O | LYS A | 525 | 6.892 | 13.789 | 87.970 | 1.00 | 24.65 | O |

| | | | | | | | | | | | |
|---|---|---|---|---|---|---|---|---|---|---|---|
| ANISOU | 1040 | O | LYS A | 525 | 3034 | 2685 | 3646 | −142 | 296 | 30 | O |

| | | | | | | | | | | |
|---|---|---|---|---|---|---|---|---|---|---|
| ATOM | 1041 | CB | LYS A | 525 | 6.677 | 11.085 | 87.430 | 1.00 | 27.03 | C |

| | | | | | | | | | | | |
|---|---|---|---|---|---|---|---|---|---|---|---|
| ANISOU | 1041 | CB | LYS A | 525 | 3332 | 3291 | 3646 | −308 | 276 | 240 | C |

| | | | | | | | | | | |
|---|---|---|---|---|---|---|---|---|---|---|
| ATOM | 1042 | CG | LYS A | 525 | 6.569 | 9.777 | 86.665 | 1.00 | 29.09 | C |

| | | | | | | | | | | | |
|---|---|---|---|---|---|---|---|---|---|---|---|
| ANISOU | 1042 | CG | LYS A | 525 | 3763 | 3577 | 3712 | −275 | 138 | 23 | C |

| | | | | | | | | | | |
|---|---|---|---|---|---|---|---|---|---|---|
| ATOM | 1043 | CD | LYS A | 525 | 5.263 | 9.009 | 86.934 | 1.00 | 31.07 | C |

| | | | | | | | | | | | |
|---|---|---|---|---|---|---|---|---|---|---|---|
| ANISOU | 1043 | CD | LYS A | 525 | 3931 | 3731 | 4144 | −391 | 169 | 102 | C |

| | | | | | | | | | | |
|---|---|---|---|---|---|---|---|---|---|---|
| ATOM | 1044 | CE | LYS A | 525 | 5.340 | 7.779 | 86.019 | 1.00 | 32.76 | C |

| | | | | | | | | | | | |
|---|---|---|---|---|---|---|---|---|---|---|---|
| ANISOU | 1044 | CE | LYS A | 525 | 4224 | 3964 | 4260 | −113 | 121 | −40 | C |

| | | | | | | | | | | |
|---|---|---|---|---|---|---|---|---|---|---|
| ATOM | 1045 | NZ | LYS A | 525 | 4.799 | 7.982 | 84.634 | 1.00 | 32.11 | N |

| | | | | | | | | | | | |
|---|---|---|---|---|---|---|---|---|---|---|---|
| ANISOU | 1045 | NZ | LYS A | 525 | 4195 | 3735 | 4271 | −247 | 160 | 134 | N |

| | | | | | | | | | | |
|---|---|---|---|---|---|---|---|---|---|---|
| ATOM | 1046 | N | THR A | 526 | 8.700 | 14.171 | 86.714 | 1.00 | 25.53 | N |

| | | | | | | | | | | | |
|---|---|---|---|---|---|---|---|---|---|---|---|
| ANISOU | 1046 | N | THR A | 526 | 3122 | 3163 | 3415 | −168 | 239 | 97 | N |

| | | | | | | | | | | |
|---|---|---|---|---|---|---|---|---|---|---|
| ATOM | 1047 | CA | THR A | 526 | 8.746 | 15.561 | 87.174 | 1.00 | 26.25 | C |

| | | | | | | | | | | | |
|---|---|---|---|---|---|---|---|---|---|---|---|
| ANISOU | 1047 | CA | THR A | 526 | 3440 | 3213 | 3320 | −8 | 257 | 25 | C |

| | | | | | | | | | | |
|---|---|---|---|---|---|---|---|---|---|---|
| ATOM | 1048 | C | THR A | 526 | 9.073 | 16.477 | 86.000 | 1.00 | 25.67 | C |

| | | | | | | | | | | | |
|---|---|---|---|---|---|---|---|---|---|---|---|
| ANISOU | 1048 | C | THR A | 526 | 3244 | 3074 | 3435 | −154 | 227 | 28 | C |

| | | | | | | | | | | |
|---|---|---|---|---|---|---|---|---|---|---|
| ATOM | 1049 | O | THR A | 526 | 9.617 | 15.957 | 85.041 | 1.00 | 24.31 | O |

| | | | | | | | | | | | |
|---|---|---|---|---|---|---|---|---|---|---|---|
| ANISOU | 1049 | O | THR A | 526 | 3257 | 2538 | 3443 | −243 | 304 | 112 | O |

| | | | | | | | | | | |
|---|---|---|---|---|---|---|---|---|---|---|
| ATOM | 1050 | CB | THR A | 526 | 9.789 | 15.899 | 88.241 | 1.00 | 27.95 | C |

| | | | | | | | | | | | |
|---|---|---|---|---|---|---|---|---|---|---|---|
| ANISOU | 1050 | CB | THR A | 526 | 3353 | 3631 | 3636 | 6 | 115 | 66 | C |

| | | | | | | | | | | |
|---|---|---|---|---|---|---|---|---|---|---|
| ATOM | 1051 | OG1 | THR A | 526 | 11.134 | 15.610 | 87.757 | 1.00 | 28.65 | O |

| | | | | | | | | | | | |
|---|---|---|---|---|---|---|---|---|---|---|---|
| ANISOU | 1051 | OG1 | THR A | 526 | 3431 | 3840 | 3617 | −23 | 231 | 38 | O |

| | | | | | | | | | | |
|---|---|---|---|---|---|---|---|---|---|---|
| ATOM | 1052 | CD2 | THR A | 526 | 9.626 | 15.163 | 89.556 | 1.00 | 29.00 | C |

| | | | | | | | | | | | |
|---|---|---|---|---|---|---|---|---|---|---|---|
| ANISOU | 1052 | CG2 | THR A | 526 | 3572 | 3855 | 3591 | −71 | 213 | 22 | C |

| | | | | | | | | | | |
|---|---|---|---|---|---|---|---|---|---|---|
| ATOM | 1053 | N | ILE A | 527 | 8.557 | 17.689 | 86.045 | 1.00 | 26.15 | N |

| | | | | | | | | | | | |
|---|---|---|---|---|---|---|---|---|---|---|---|
| ANISOU | 1053 | N | ILE A | 527 | 3520 | 2973 | 3442 | −183 | 293 | 93 | N |

| | | | | | | | | | | |
|---|---|---|---|---|---|---|---|---|---|---|
| ATOM | 1054 | CA | ILE A | 527 | 8.913 | 18.681 | 85.013 | 1.00 | 25.72 | C |

| | | | | | | | | | | | |
|---|---|---|---|---|---|---|---|---|---|---|---|
| ANISOU | 1054 | CA | ILE A | 527 | 3438 | 3005 | 3331 | −268 | 311 | −10 | C |

| | | | | | | | | | | |
|---|---|---|---|---|---|---|---|---|---|---|
| ATOM | 1055 | C | ILE A | 527 | 9.538 | 19.836 | 85.805 | 1.00 | 25.92 | C |

| | | | | | | | | | | | |
|---|---|---|---|---|---|---|---|---|---|---|---|
| ANISOU | 1055 | C | ILE A | 527 | 3337 | 2983 | 3528 | −269 | 322 | −38 | C |

| | | | | | | | | | | |
|---|---|---|---|---|---|---|---|---|---|---|
| ATOM | 1056 | O | ILE A | 527 | 8.884 | 20.394 | 86.696 | 1.00 | 25.19 | O |

| | | | | | | | | | | | |
|---|---|---|---|---|---|---|---|---|---|---|---|
| ANISOU | 1056 | O | ILE A | 527 | 3346 | 2724 | 3502 | −384 | 382 | 36 | O |

| | | | | | | | | | | |
|---|---|---|---|---|---|---|---|---|---|---|
| ATOM | 1057 | CB | ILE A | 527 | 7.782 | 19.182 | 84.149 | 1.00 | 27.76 | C |

| | | | | | | | | | | | |
|---|---|---|---|---|---|---|---|---|---|---|---|
| ANISOU | 1057 | CB | ILE A | 527 | 3756 | 3288 | 3503 | −198 | 185 | 118 | C |

| | | | | | | | | | | |
|---|---|---|---|---|---|---|---|---|---|---|
| ATOM | 1058 | CG1 | ILE A | 527 | 7.127 | 17.969 | 83.496 | 1.00 | 28.22 | C |

| | | | | | | | | | | | |
|---|---|---|---|---|---|---|---|---|---|---|---|
| ANISOU | 1058 | CG1 | ILE A | 527 | 3810 | 3383 | 3529 | −198 | −1 | 116 | C |

| | | | | | | | | | | |
|---|---|---|---|---|---|---|---|---|---|---|
| ATOM | 1059 | CG2 | ILE A | 527 | 8.299 | 20.211 | 83.130 | 1.00 | 27.06 | C |

| | | | | | | | | | | | |
|---|---|---|---|---|---|---|---|---|---|---|---|
| ANISOU | 1059 | CG2 | ILE A | 527 | 3894 | 3026 | 3359 | −121 | 105 | 49 | C |

| | | | | | | | | | | |
|---|---|---|---|---|---|---|---|---|---|---|
| ATOM | 1060 | CD1 | ILE A | 527 | 6.196 | 18.128 | 82.355 | 1.00 | 28.80 | C |

| | | | | | | | | | | | |
|---|---|---|---|---|---|---|---|---|---|---|---|
| ANISOU | 1060 | CD1 | ILE A | 527 | 3371 | 3693 | 3879 | −214 | −27 | 149 | C |

| | | | | | | | | | | |
|---|---|---|---|---|---|---|---|---|---|---|
| ATOM | 1061 | N | GLY A | 528 | 10.773 | 20.197 | 85.417 | 1.00 | 23.68 | N |

| | | | | | | | | | | | |
|---|---|---|---|---|---|---|---|---|---|---|---|
| ANISOU | 1061 | N | GLY A | 528 | 3179 | 2589 | 3230 | −100 | 322 | −67 | N |

| | | | | | | | | | | |
|---|---|---|---|---|---|---|---|---|---|---|
| ATOM | 1062 | CA | GLY A | 528 | 11.439 | 21.235 | 86.200 | 1.00 | 22.81 | C |

| | | | | | | | | | | | |
|---|---|---|---|---|---|---|---|---|---|---|---|
| ANISOU | 1062 | CA | GLY A | 528 | 3073 | 2565 | 3030 | −163 | 179 | 105 | C |

| | | | | | | | | | | |
|---|---|---|---|---|---|---|---|---|---|---|
| ATOM | 1063 | C | GLY A | 528 | 11.870 | 22.408 | 85.327 | 1.00 | 21.82 | C |

| | | | | | | | | | | | |
|---|---|---|---|---|---|---|---|---|---|---|---|
| ANISOU | 1083 | C | GLY A | 528 | 2935 | 2571 | 2784 | −210 | 302 | 13 | C |

| | | | | | | | | | | |
|---|---|---|---|---|---|---|---|---|---|---|
| ATOM | 1064 | O | GLY A | 528 | 12.145 | 22.227 | 84.130 | 1.00 | 21.67 | O |

| | | | | | | | | | | | |
|---|---|---|---|---|---|---|---|---|---|---|---|
| ANISOU | 1064 | O | GLY A | 528 | 3434 | 2090 | 2709 | −316 | 324 | 118 | O |

| | | | | | | | | | | |
|---|---|---|---|---|---|---|---|---|---|---|
| ATOM | 1065 | N | TYR A | 529 | 11.827 | 23.570 | 85.943 | 1.00 | 19.07 | N |

| | | | | | | | | | | | |
|---|---|---|---|---|---|---|---|---|---|---|---|
| ANISOU | 1065 | N | TYR A | 529 | 2557 | 2264 | 2423 | −228 | 432 | 288 | N |

| | | | | | | | | | | |
|---|---|---|---|---|---|---|---|---|---|---|
| ATOM | 1066 | CA | TYR A | 529 | 12.249 | 24.815 | 85.244 | 1.00 | 19.32 | C |

| | | | | | | | | | | | |
|---|---|---|---|---|---|---|---|---|---|---|---|
| ANIS0U | 1066 | CA | TYR A | 529 | 2534 | 2140 | 2667 | −71 | 173 | 302 | C |

| | | | | | | | | | | |
|---|---|---|---|---|---|---|---|---|---|---|
| ATOM | 1067 | C | TYR A | 529 | 13.436 | 25.315 | 86.040 | 1.00 | 20.01 | C |

| | | | | | | | | | | | |
|---|---|---|---|---|---|---|---|---|---|---|---|
| ANISOU | 1067 | C | TYR A | 529 | 2649 | 2308 | 2646 | −15 | 174 | 67 | C |

| | | | | | | | | | | |
|---|---|---|---|---|---|---|---|---|---|---|
| ATOM | 1066 | O | TYR A | 529 | 13.318 | 25.491 | 87.260 | 1.00 | 20.14 | O |

| | | | | | | | | | | | |
|---|---|---|---|---|---|---|---|---|---|---|---|
| ANISOU | 1068 | O | TYR A | 529 | 2515 | 2533 | 2604 | −316 | 392 | 139 | O |

| | | | | | | | | | | |
|---|---|---|---|---|---|---|---|---|---|---|
| ATOM | 1069 | CB | TYR A | 529 | 11.101 | 25.844 | 85.232 | 1.00 | 19.92 | C |

| | | | | | | | | | | | |
|---|---|---|---|---|---|---|---|---|---|---|---|
| ANISOU | 1069 | CB | TYR A | 529 | 2567 | 2258 | 2741 | −13 | 180 | 66 | C |

| | | | | | | | | | | |
|---|---|---|---|---|---|---|---|---|---|---|
| ATOM | 1070 | CG | TYR A | 529 | 11.593 | 27.177 | 84.663 | 1.00 | 19.55 | C |

| | | | | | | | | | | | |
|---|---|---|---|---|---|---|---|---|---|---|---|
| ANISOU | 1070 | CG | TYR A | 529 | 2497 | 2303 | 2629 | −87 | 180 | 65 | C |

| | | | | | | | | | | |
|---|---|---|---|---|---|---|---|---|---|---|
| ATOM | 1071 | CD1 | TYR A | 529 | 11.836 | 27.317 | 83.300 | 1.00 | 19.22 | C |

| | | | | | | | | | | | |
|---|---|---|---|---|---|---|---|---|---|---|---|
| ANISOU | 1071 | CD1 | TYR A | 529 | 2345 | 2387 | 2573 | −129 | 118 | 52 | C |

| | | | | | | | | | | |
|---|---|---|---|---|---|---|---|---|---|---|
| ATOM | 1072 | CD2 | TYR A | 529 | 11.825 | 28.242 | 85.498 | 1.00 | 21.21 | C |

| | | | | | | | | | | | |
|---|---|---|---|---|---|---|---|---|---|---|---|
| ANISOU | 1072 | CD2 | TYR A | 529 | 2756 | 2502 | 2800 | −136 | 192 | −9 | C |

| | | | | | | | | | | |
|---|---|---|---|---|---|---|---|---|---|---|
| ATOM | 1073 | CE1 | TYR A | 529 | 12.275 | 28.523 | 82.766 | 1.00 | 19.18 | C |

| | | | | | | | | | | | |
|---|---|---|---|---|---|---|---|---|---|---|---|
| ANISOU | 1073 | CE1 | TYR A | 529 | 2320 | 2392 | 2573 | −195 | 69 | −36 | C |

| | | | | | | | | | | |
|---|---|---|---|---|---|---|---|---|---|---|
| ATOM | 1074 | CE2 | TYR A | 529 | 12.292 | 29.454 | 84.977 | 1.00 | 20.66 | C |

| | | | | | | | | | | | |
|---|---|---|---|---|---|---|---|---|---|---|---|
| ANISOU | 1074 | CE2 | TYR A | 529 | 2508 | 2470 | 2873 | −167 | 199 | 44 | C |

| | | | | | | | | | | |
|---|---|---|---|---|---|---|---|---|---|---|
| ATOM | 1075 | CZ | TYR A | 529 | 12.478 | 29.588 | 83.629 | 1.00 | 19.62 | C |

| | | | | | | | | | | | |
|---|---|---|---|---|---|---|---|---|---|---|---|
| ANISOU | 1075 | CZ | TYR A | 529 | 2545 | 2147 | 2763 | −154 | 133 | −1 | C |

| | | | | | | | | | | |
|---|---|---|---|---|---|---|---|---|---|---|
| ATOM | 1076 | OH | TYR A | 529 | 12.986 | 30.765 | 83.079 | 1.00 | 20.48 | O |

| | | | | | | | | | | | |
|---|---|---|---|---|---|---|---|---|---|---|---|
| ANISOU | 1076 | OH | TYR A | 529 | 2570 | 2316 | 2897 | −321 | 401 | 29 | O |

| | | | | | | | | | | |
|---|---|---|---|---|---|---|---|---|---|---|
| ATOM | 1077 | N | VAL A | 530 | 14.599 | 25.509 | 85.419 | 1.00 | 19.71 | N |

| | | | | | | | | | | | |
|---|---|---|---|---|---|---|---|---|---|---|---|
| ANISOU | 1077 | N | VAL A | 530 | 2583 | 2184 | 2721 | −161 | 244 | 215 | N |

| | | | | | | | | | | |
|---|---|---|---|---|---|---|---|---|---|---|
| ATOM | 1078 | CA | VAL A | 530 | 15.818 | 25.864 | 86.151 | 1.00 | 19.51 | C |

| | | | | | | | | | | | |
|---|---|---|---|---|---|---|---|---|---|---|---|
| ANISOU | 1078 | CA | VAL A | 530 | 2619 | 2202 | 2591 | −49 | 261 | 158 | C |

| | | | | | | | | | | |
|---|---|---|---|---|---|---|---|---|---|---|
| ATOM | 1079 | C | VAL A | 530 | 16.382 | 27.118 | 85.501 | 1.00 | 18.62 | C |

| | | | | | | | | | | | |
|---|---|---|---|---|---|---|---|---|---|---|---|
| ANISOU | 1079 | C | VAL A | 530 | 2445 | 2200 | 2429 | −134 | 225 | 21 | C |

| | | | | | | | | | | |
|---|---|---|---|---|---|---|---|---|---|---|
| ATOM | 1080 | O | VAL A | 530 | 16.599 | 27.123 | 84.306 | 1.00 | 19.34 | O |

| | | | | | | | | | | | |
|---|---|---|---|---|---|---|---|---|---|---|---|
| ANISOU | 1080 | O | VAL A | 530 | 2687 | 2294 | 2367 | −271 | 166 | 61 | O |

| | | | | | | | | | | |
|---|---|---|---|---|---|---|---|---|---|---|
| ATOM | 1081 | CB | VAL A | 530 | 16.807 | 24.679 | 86.249 | 1.00 | 21.91 | C |

| | | | | | | | | | | | |
|---|---|---|---|---|---|---|---|---|---|---|---|
| ANISOU | 1081 | CB | VAL A | 530 | 2857 | 2345 | 3124 | −36 | 187 | 52 | C |

| | | | | | | | | | | |
|---|---|---|---|---|---|---|---|---|---|---|
| ATOM | 1082 | CG1 | VAL A | 530 | 17.413 | 24.267 | 84.933 | 1.00 | 24.00 | C |

| | | | | | | | | | | | |
|---|---|---|---|---|---|---|---|---|---|---|---|
| ANISOU | 1082 | CG1 | VAL A | 530 | 3085 | 2880 | 3154 | 43 | 244 | 34 | C |

| | | | | | | | | | | |
|---|---|---|---|---|---|---|---|---|---|---|
| ATOM | 1083 | CG2 | VAL A | 530 | 17.857 | 24.947 | 87.297 | 1.00 | 22.89 | C |

| | | | | | | | | | | | |
|---|---|---|---|---|---|---|---|---|---|---|---|
| ANISOU | 1083 | CG2 | VAL A | 530 | 2902 | 2691 | 3104 | 182 | 80 | 1 | C |

| | | | | | | | | | | |
|---|---|---|---|---|---|---|---|---|---|---|
| ATOM | 1084 | N | ASP A | 531 | 16.541 | 28.148 | 86.313 | 1.00 | 18.01 | N |

| | | | | | | | | | | | |
|---|---|---|---|---|---|---|---|---|---|---|---|
| ANISOU | 1084 | N | ASP A | 531 | 2426 | 1966 | 2452 | −183 | 389 | 124 | N |

| | | | | | | | | | | |
|---|---|---|---|---|---|---|---|---|---|---|
| ATOM | 1085 | CA | ASP A | 531 | 16.922 | 29.466 | 85.757 | 1.00 | 17.65 | C |

| | | | | | | | | | | | |
|---|---|---|---|---|---|---|---|---|---|---|---|
| ANISOU | 1085 | CA | ASP A | 531 | 2484 | 1877 | 2344 | −179 | 246 | 29 | C |

| | | | | | | | | | | |
|---|---|---|---|---|---|---|---|---|---|---|
| ATOM | 1086 | C | ASP A | 531 | 18.005 | 30.005 | 86.684 | 1.00 | 17.93 | C |

| | | | | | | | | | | | |
|---|---|---|---|---|---|---|---|---|---|---|---|
| ANISOU | 1086 | C | ASP A | 531 | 2407 | 2089 | 2317 | −161 | 142 | 112 | C |

| | | | | | | | | | | |
|---|---|---|---|---|---|---|---|---|---|---|
| ATOM | 1087 | O | ASP A | 531 | 17.778 | 30.190 | 87.888 | 1.00 | 19.21 | O |

| | | | | | | | | | | | |
|---|---|---|---|---|---|---|---|---|---|---|---|
| ANISOU | 1087 | O | ASP A | 531 | 2771 | 2146 | 2384 | −125 | 233 | −33 | O |

| | | | | | | | | | | |
|---|---|---|---|---|---|---|---|---|---|---|
| ATOM | 1088 | CB | ASP A | 531 | 15.637 | 30.285 | 85.686 | 1.00 | 18.96 | C |

| | | | | | | | | | | | |
|---|---|---|---|---|---|---|---|---|---|---|---|
| ANISOU | 1088 | CB | ASP A | 531 | 2561 | 2170 | 2472 | −54 | 84 | 46 | C |

| | | | | | | | | | | |
|---|---|---|---|---|---|---|---|---|---|---|
| ATOM | 1089 | CG | ASP A | 531 | 15.766 | 31.672 | 85.084 | 1.00 | 19.66 | C |

| | | | | | | | | | | | |
|---|---|---|---|---|---|---|---|---|---|---|---|
| ANISOU | 1089 | CG | ASP A | 531 | 2837 | 2185 | 2448 | 25 | 86 | 15 | C |

| | | | | | | | | | | |
|---|---|---|---|---|---|---|---|---|---|---|
| ATOM | 1090 | OD1 | ASP A | 531 | 16.844 | 32.274 | 85.205 | 1.00 | 20.25 | O |

| | | | | | | | | | | | |
|---|---|---|---|---|---|---|---|---|---|---|---|
| ANISOU | 1090 | OD1 | ASP A | 531 | 2842 | 2265 | 2585 | 2 | 151 | 94 | O |

| | | | | | | | | | | |
|---|---|---|---|---|---|---|---|---|---|---|
| ATOM | 1091 | OD2 | ASP A | 531 | 14.731 | 32.150 | 84.536 | 1.00 | 19.95 | O |

| | | | | | | | | | | | |
|---|---|---|---|---|---|---|---|---|---|---|---|
| ANISOU | 1091 | OD2 | ASP A | 531 | 2846 | 2166 | 2567 | −31 | 43 | 240 | O |

| | | | | | | | | | | |
|---|---|---|---|---|---|---|---|---|---|---|
| ATOM | 1092 | N | SER A | 532 | 19.120 | 30.473 | 86.080 | 1.00 | 17.40 | N |

| | | | | | | | | | | | |
|---|---|---|---|---|---|---|---|---|---|---|---|
| ANISOU | 1092 | N | SER A | 532 | 2264 | 1902 | 2446 | −72 | 297 | −76 | N |

| | | | | | | | | | | |
|---|---|---|---|---|---|---|---|---|---|---|
| ATOM | 1093 | CA | SER A | 532 | 20.242 | 30.997 | 86.832 | 1.00 | 18.39 | C |

| | | | | | | | | | | | |
|---|---|---|---|---|---|---|---|---|---|---|---|
| ANISOU | 1093 | CA | SER A | 532 | 2611 | 2139 | 2239 | 65 | 64 | −49 | C |

| | | | | | | | | | | |
|---|---|---|---|---|---|---|---|---|---|---|
| ATOM | 1094 | C | SER A | 532 | 20.136 | 32.508 | 87.153 | 1.00 | 18.84 | C |

| | | | | | | | | | | | |
|---|---|---|---|---|---|---|---|---|---|---|---|
| ANISOU | 1094 | C | SER A | 532 | 2556 | 2203 | 2400 | −177 | 156 | −105 | C |

| | | | | | | | | | | |
|---|---|---|---|---|---|---|---|---|---|---|
| ATOM | 1095 | O | SER A | 532 | 21.073 | 33.027 | 87.750 | 1.00 | 18.74 | O |

| | | | | | | | | | | | |
|---|---|---|---|---|---|---|---|---|---|---|---|
| ANISOU | 1095 | O | SER A | 532 | 2671 | 2009 | 2441 | −196 | 152 | −251 | O |

| | | | | | | | | | | |
|---|---|---|---|---|---|---|---|---|---|---|
| ATOM | 1096 | CB | SER A | 532 | 21.592 | 30.748 | 86.133 | 1.00 | 17.93 | C |

| | | | | | | | | | | | |
|---|---|---|---|---|---|---|---|---|---|---|---|
| ANISOU | 1096 | CB | SER A | 532 | 2371 | 2432 | 2008 | −23 | 14 | 97 | C |

| | | | | | | | | | | |
|---|---|---|---|---|---|---|---|---|---|---|
| ATOM | 1097 | OG | SER A | 532 | 21.558 | 31.336 | 84.814 | 1.00 | 17.00 | O |

| | | | | | | | | | | | |
|---|---|---|---|---|---|---|---|---|---|---|---|
| ANISOU | 1097 | OG | SER A | 532 | 2459 | 2032 | 1970 | 45 | 31 | 69 | O |

| | | | | | | | | | | |
|---|---|---|---|---|---|---|---|---|---|---|
| ATOM | 1098 | N | LEU A | 533 | 19.032 | 33.127 | 86.821 | 1.00 | 19.85 | N |

| | | | | | | | | | | | |
|---|---|---|---|---|---|---|---|---|---|---|---|
| ANISOU | 1098 | N | LEU A | 533 | 2745 | 2151 | 2645 | −51 | 165 | −141 | N |

| | | | | | | | | | | |
|---|---|---|---|---|---|---|---|---|---|---|
| ATOM | 1099 | CA | LEU A | 533 | 18.818 | 34.515 | 87.294 | 1.00 | 21.71 | C |

| | | | | | | | | | | | |
|---|---|---|---|---|---|---|---|---|---|---|---|
| ANISOU | 1099 | CA | LEU A | 533 | 3000 | 2246 | 3002 | 103 | 143 | −166 | C |

| | | | | | | | | | | |
|---|---|---|---|---|---|---|---|---|---|---|
| ATOM | 1100 | C | LEU A | 533 | 17.338 | 34.623 | 87.615 | 1.00 | 23.14 | C |

| | | | | | | | | | | | |
|---|---|---|---|---|---|---|---|---|---|---|---|
| ANISOU | 1100 | C | LEU A | 533 | 2965 | 2634 | 3193 | −16 | 25 | −200 | C |

| | | | | | | | | | | |
|---|---|---|---|---|---|---|---|---|---|---|
| ATOM | 1101 | O | LEU A | 533 | 16.559 | 35.275 | 86.940 | 1.00 | 24.86 | O |

| | | | | | | | | | | | |
|---|---|---|---|---|---|---|---|---|---|---|---|
| ANISOU | 1101 | O | LEU A | 533 | 3149 | 3073 | 3224 | 203 | −39 | −264 | O |

| | | | | | | | | | | |
|---|---|---|---|---|---|---|---|---|---|---|
| ATOM | 1102 | CB | LEU A | 533 | 19.395 | 35.556 | 86.329 | 1.00 | 24.34 | C |

| | | | | | | | | | | | |
|---|---|---|---|---|---|---|---|---|---|---|---|
| ANISOU | 1102 | CB | LEU A | 533 | 3307 | 2753 | 3186 | −43 | 112 | 53 | C |

| | | | | | | | | | | |
|---|---|---|---|---|---|---|---|---|---|---|
| ATOM | 1103 | CG | LEU A | 533 | 19.592 | 36.950 | 86.969 | 1.00 | 27.67 | C |

| | | | | | | | | | | | |
|---|---|---|---|---|---|---|---|---|---|---|---|
| ANISOU | 1103 | CG | LEU A | 533 | 3878 | 3059 | 3577 | −9 | −7 | −174 | C |

| | | | | | | | | | | |
|---|---|---|---|---|---|---|---|---|---|---|
| ATOM | 1104 | CD1 | LEU A | 533 | 20.617 | 36.899 | 88.082 | 1.00 | 28.13 | C |

| | | | | | | | | | | | |
|---|---|---|---|---|---|---|---|---|---|---|---|
| ANISOU | 1104 | CD1 | LEU A | 533 | 3716 | 3229 | 3745 | −53 | −34 | −148 | C |

| | | | | | | | | | | |
|---|---|---|---|---|---|---|---|---|---|---|
| ATOM | 1105 | CD2 | LEU A | 533 | 20.033 | 37.930 | 85.892 | 1.00 | 29.92 | C |

| | | | | | | | | | | | |
|---|---|---|---|---|---|---|---|---|---|---|---|
| ANISOU | 1105 | CD2 | LEU A | 533 | 3970 | 3511 | 3887 | −111 | 88 | 17 | C |

| | | | | | | | | | | |
|---|---|---|---|---|---|---|---|---|---|---|
| ATOM | 1106 | N | SER A | 534 | 16.952 | 33.948 | 88.691 | 1.00 | 25.11 | N |

| | | | | | | | | | | | |
|---|---|---|---|---|---|---|---|---|---|---|---|
| ANISOU | 1106 | N | SER A | 534 | 3066 | 3104 | 3372 | 7 | 133 | −128 | N |

| | | | | | | | | | | |
|---|---|---|---|---|---|---|---|---|---|---|
| ATOM | 1107 | CA | SER A | 534 | 15.539 | 33.920 | 89.094 | 1.00 | 29.12 | C |

| | | | | | | | | | | | |
|---|---|---|---|---|---|---|---|---|---|---|---|
| ANISOU | 1107 | CA | SER A | 534 | 3196 | 3881 | 3988 | −80 | 190 | 9 | C |

| | | | | | | | | | | |
|---|---|---|---|---|---|---|---|---|---|---|
| ATOM | 1108 | C | SER A | 534 | 15.349 | 34.300 | 90.550 | 1.00 | 33.77 | C |

| | | | | | | | | | | | |
|---|---|---|---|---|---|---|---|---|---|---|---|
| ANISOU | 1108 | C | SER A | 534 | 4280 | 4419 | 4134 | −194 | 87 | −119 | C |

| | | | | | | | | | | |
|---|---|---|---|---|---|---|---|---|---|---|
| ATOM | 1109 | O | SER A | 534 | 16.094 | 33.806 | 91.401 | 1.00 | 34.44 | O |

| | | | | | | | | | | | |
|---|---|---|---|---|---|---|---|---|---|---|---|
| ANISOU | 1109 | O | SER A | 534 | 4366 | 4668 | 4052 | −165 | 174 | −72 | O |

| | | | | | | | | | | |
|---|---|---|---|---|---|---|---|---|---|---|
| ATOM | 1110 | CB | SER A | 534 | 15.093 | 32.458 | 88.916 | 1.00 | 29.92 | C |

| | | | | | | | | | | | |
|---|---|---|---|---|---|---|---|---|---|---|---|
| ANISOU | 1110 | CB | SER A | 534 | 3380 | 3929 | 4061 | −138 | 236 | −206 | C |

| | | | | | | | | | | |
|---|---|---|---|---|---|---|---|---|---|---|
| ATOM | 1111 | OG | SER A | 534 | 13.809 | 32.243 | 89.499 | 1.00 | 30.70 | O |

| | | | | | | | | | | | |
|---|---|---|---|---|---|---|---|---|---|---|---|
| ANISOU | 1111 | OG | SER A | 534 | 3575 | 3946 | 4142 | −250 | 236 | −478 | O |

| | | | | | | | | | | |
|---|---|---|---|---|---|---|---|---|---|---|
| ATOM | 1112 | N | ASN A | 535 | 14.266 | 35.000 | 90.853 | 1.00 | 38.52 | N |

| | | | | | | | | | | | |
|---|---|---|---|---|---|---|---|---|---|---|---|
| ANISOU | 1112 | N | ASN A | 535 | 4635 | 4858 | 5144 | −4 | 91 | −59 | N |

| | | | | | | | | | | |
|---|---|---|---|---|---|---|---|---|---|---|
| ATOM | 1113 | CA | ASN A | 535 | 13.945 | 35.281 | 92.263 | 1.00 | 42.88 | C |

| | | | | | | | | | | | |
|---|---|---|---|---|---|---|---|---|---|---|---|
| ANISOU | 1113 | CA | ASN A | 535 | 5476 | 5575 | 5240 | −52 | 104 | −85 | C |

| | | | | | | | | | | |
|---|---|---|---|---|---|---|---|---|---|---|
| ATOM | 1114 | C | ASN A | 535 | 12.969 | 34.223 | 92.783 | 1.00 | 43.94 | C |

| | | | | | | | | | | | |
|---|---|---|---|---|---|---|---|---|---|---|---|
| ANISOU | 1114 | C | ASN A | 535 | 5602 | 5574 | 5519 | −83 | 98 | −69 | C |

| | | | | | | | | | | |
|---|---|---|---|---|---|---|---|---|---|---|
| ATOM | 1115 | O | ASN A | 535 | 12.632 | 34.188 | 93.981 | 1.00 | 45.16 | O |

| | | | | | | | | | | | |
|---|---|---|---|---|---|---|---|---|---|---|---|
| ANISOU | 1115 | O | ASN A | 535 | 5794 | 5834 | 5529 | −62 | 126 | −57 | O |

| | | | | | | | | | | |
|---|---|---|---|---|---|---|---|---|---|---|
| ATOM | 1116 | CB | ASN A | 535 | 13.426 | 36.693 | 92.496 | 1.00 | 45.09 | C |

| | | | | | | | | | | | |
|---|---|---|---|---|---|---|---|---|---|---|---|
| ANISOU | 1116 | CB | ASN A | 535 | 5725 | 5679 | 5729 | 67 | 19 | −85 | C |

| | | | | | | | | | | |
|---|---|---|---|---|---|---|---|---|---|---|
| ATOM | 1117 | CG | ASN A | 535 | 14.484 | 37.713 | 92.100 | 1.00 | 46.65 | C |

| | | | | | | | | | | | |
|---|---|---|---|---|---|---|---|---|---|---|---|
| ANISOU | 1117 | CG | ASN A | 535 | 5922 | 5870 | 5934 | −51 | 50 | −47 | C |

| | | | | | | | | | | |
|---|---|---|---|---|---|---|---|---|---|---|
| ATOM | 1118 | OD1 | ASN A | 535 | 15.647 | 37.604 | 92.509 | 1.00 | 47.97 | O |

| | | | | | | | | | | | |
|---|---|---|---|---|---|---|---|---|---|---|---|
| ANISOU | 1118 | OD1 | ASN A | 535 | 6025 | 6115 | 6085 | −9 | −45 | −24 | O |

| | | | | | | | | | | |
|---|---|---|---|---|---|---|---|---|---|---|
| ATOM | 1119 | ND2 | ASN A | 535 | 14.074 | 38.675 | 91.291 | 1.00 | 47.55 | N |

| | | | | | | | | | | | |
|---|---|---|---|---|---|---|---|---|---|---|---|
| ANISOU | 1119 | ND2 | ASN A | 535 | 6070 | 5977 | 6019 | −7 | −34 | −26 | N |

| | | | | | | | | | | |
|---|---|---|---|---|---|---|---|---|---|---|
| ATOM | 1120 | N | GLY A | 536 | 12.547 | 33.322 | 91.888 | 1.00 | 43.16 | N |

| | | | | | | | | | | | |
|---|---|---|---|---|---|---|---|---|---|---|---|
| ANISOU | 1120 | N | GLY A | 536 | 5433 | 5605 | 5360 | −84 | 167 | −75 | N |

| | | | | | | | | | | |
|---|---|---|---|---|---|---|---|---|---|---|
| ATOM | 1121 | CA | GLY A | 536 | 11.720 | 32.219 | 92.314 | 1.00 | 42.57 | C |

| | | | | | | | | | | | |
|---|---|---|---|---|---|---|---|---|---|---|---|
| ANISOU | 1121 | CA | GLY A | 536 | 5433 | 5440 | 5300 | −46 | 75 | −137 | C |

| | | | | | | | | | | |
|---|---|---|---|---|---|---|---|---|---|---|
| ATOM | 1122 | C | GLY A | 536 | 10.448 | 32.026 | 91.523 | 1.00 | 41.44 | C |

| | | | | | | | | | | | |
|---|---|---|---|---|---|---|---|---|---|---|---|
| ANISOU | 1122 | C | GLY A | 536 | 5306 | 5268 | 5172 | 28 | 180 | −107 | C |

| | | | | | | | | | | |
|---|---|---|---|---|---|---|---|---|---|---|
| ATOM | 1123 | O | GLY A | 536 | 10.131 | 32.713 | 90.550 | 1.00 | 39.62 | O |

| | | | | | | | | | | | |
|---|---|---|---|---|---|---|---|---|---|---|---|
| ANISOU | 1123 | O | GLY A | 536 | 4959 | 4833 | 5261 | 114 | 206 | −212 | O |

| | | | | | | | | | | |
|---|---|---|---|---|---|---|---|---|---|---|
| ATOM | 1124 | N | PRO A | 537 | 9.723 | 30.984 | 91.954 | 1.00 | 41.72 | N |

| | | | | | | | | | | | |
|---|---|---|---|---|---|---|---|---|---|---|---|
| ANISOU | 1124 | N | PRO A | 537 | 5401 | 5191 | 5259 | −40 | 70 | −135 | N |

| | | | | | | | | | | |
|---|---|---|---|---|---|---|---|---|---|---|
| ATOM | 1125 | CA | PRO A | 537 | 8.483 | 30.615 | 91.290 | 1.00 | 41.96 | C |

| | | | | | | | | | | | |
|---|---|---|---|---|---|---|---|---|---|---|---|
| ANISOU | 1125 | CA | PRO A | 537 | 5303 | 5353 | 5288 | −2 | 71 | −64 | C |

| | | | | | | | | | | |
|---|---|---|---|---|---|---|---|---|---|---|
| ATOM | 1126 | C | PRO A | 537 | 7.735 | 31.886 | 90.963 | 1.00 | 42.20 | C |

| | | | | | | | | | | | |
|---|---|---|---|---|---|---|---|---|---|---|---|
| ANISOU | 1126 | C | PRO A | 537 | 5449 | 5319 | 5267 | −37 | 26 | 13 | C |

| | | | | | | | | | | |
|---|---|---|---|---|---|---|---|---|---|---|
| ATOM | 1127 | O | PRO A | 537 | 7.541 | 32.752 | 91.840 | 1.00 | 43.58 | O |

| | | | | | | | | | | | |
|---|---|---|---|---|---|---|---|---|---|---|---|
| ANISOU | 1127 | O | PRO A | 537 | 5645 | 5509 | 5405 | −72 | 73 | −119 | O |

| | | | | | | | | | | |
|---|---|---|---|---|---|---|---|---|---|---|
| ATOM | 1128 | CB | PRO A | 537 | 7.775 | 29.709 | 92.267 | 1.00 | 41.74 | C |

| | | | | | | | | | | | |
|---|---|---|---|---|---|---|---|---|---|---|---|
| ANISOU | 1128 | CB | PRO A | 537 | 5338 | 5221 | 5301 | −24 | 57 | −95 | C |

| | | | | | | | | | | |
|---|---|---|---|---|---|---|---|---|---|---|
| ATOM | 1129 | CG | PRO A | 537 | 8.837 | 29.154 | 93.161 | 1.00 | 42.31 | C |

| | | | | | | | | | | | |
|---|---|---|---|---|---|---|---|---|---|---|---|
| ANISOU | 1129 | CG | PRO A | 537 | 5387 | 5305 | 5385 | −51 | 26 | −60 | C |

| | | | | | | | | | | |
|---|---|---|---|---|---|---|---|---|---|---|
| ATOM | 1130 | CD | PRO A | 537 | 10.021 | 30.077 | 93.079 | 1.00 | 41.98 | C |

| | | | | | | | | | | | |
|---|---|---|---|---|---|---|---|---|---|---|---|
| ANISOU | 1130 | CD | PRO A | 537 | 5279 | 5392 | 5279 | −36 | 56 | −94 | C |

| | | | | | | | | | | |
|---|---|---|---|---|---|---|---|---|---|---|
| ATOM | 1131 | N | ASN A | 538 | 7.373 | 32.010 | 89.898 | 1.00 | 40.61 | N |

| | | | | | | | | | | | |
|---|---|---|---|---|---|---|---|---|---|---|---|
| ANISOU | 1131 | N | ASN A | 538 | 5152 | 5124 | 5155 | −133 | 72 | −84 | N |

| | | | | | | | | | | |
|---|---|---|---|---|---|---|---|---|---|---|
| ATOM | 1132 | CA | ASN A | 538 | 6.685 | 33.207 | 89.241 | 1.00 | 40.39 | C |

| | | | | | | | | | | | |
|---|---|---|---|---|---|---|---|---|---|---|---|
| ANISOU | 1132 | CA | ASN A | 538 | 5000 | 5184 | 5163 | −68 | 83 | −29 | C |

| | | | | | | | | | | |
|---|---|---|---|---|---|---|---|---|---|---|
| ATOM | 1133 | C | ASN A | 538 | 5.686 | 32.746 | 88.191 | 1.00 | 40.25 | C |

| | | | | | | | | | | | |
|---|---|---|---|---|---|---|---|---|---|---|---|
| ANISOU | 1133 | C | ASN A | 538 | 5117 | 5067 | 5110 | −62 | 90 | −49 | C |

| | | | | | | | | | | |
|---|---|---|---|---|---|---|---|---|---|---|
| ATOM | 1134 | O | ASN A | 538 | 5.601 | 31.531 | 87.935 | 1.00 | 38.37 | O |

| | | | | | | | | | | | |
|---|---|---|---|---|---|---|---|---|---|---|---|
| ANISOU | 1134 | O | ASN A | 538 | 4756 | 5040 | 4784 | −16 | 181 | −116 | O |

| | | | | | | | | | | |
|---|---|---|---|---|---|---|---|---|---|---|
| ATOM | 1135 | CB | ASN A | 538 | 7.631 | 34.252 | 88.746 | 1.00 | 40.79 | C |

| | | | | | | | | | | | |
|---|---|---|---|---|---|---|---|---|---|---|---|
| ANISOU | 1135 | CB | ASN A | 538 | 5146 | 5171 | 5181 | −104 | 40 | −56 | C |

| | | | | | | | | | | |
|---|---|---|---|---|---|---|---|---|---|---|
| ATOM | 1136 | CG | ASN A | 538 | 8.113 | 34.344 | 87.324 | 1.00 | 40.60 | C |

| | | | | | | | | | | | |
|---|---|---|---|---|---|---|---|---|---|---|---|
| ANISOU | 1136 | CG | ASN A | 538 | 4958 | 5219 | 5247 | −122 | 124 | −86 | C |

| | | | | | | | | | | |
|---|---|---|---|---|---|---|---|---|---|---|
| ATOM | 1137 | OD1 | ASN A | 538 | 7.702 | 33.768 | 86.304 | 1.00 | 39.52 | O |

| | | | | | | | | | | | |
|---|---|---|---|---|---|---|---|---|---|---|---|
| ANISOU | 1137 | OD1 | ASN A | 538 | 4663 | 5054 | 5297 | −206 | 276 | −173 | O |

| | | | | | | | | | | |
|---|---|---|---|---|---|---|---|---|---|---|
| ATOM | 1138 | ND2 | ASN A | 538 | 9.072 | 35.272 | 87.179 | 1.00 | 39.93 | N |

| | | | | | | | | | | | |
|---|---|---|---|---|---|---|---|---|---|---|---|
| ANISOU | 1138 | ND2 | ASN A | 538 | 4863 | 5104 | 5205 | −82 | 84 | −126 | N |

| | | | | | | | | | | |
|---|---|---|---|---|---|---|---|---|---|---|
| ATOM | 1139 | N | ALA A | 539 | 4.879 | 33.635 | 87.608 | 1.00 | 40.78 | N |

| | | | | | | | | | | | |
|---|---|---|---|---|---|---|---|---|---|---|---|
| ANISOU | 1139 | N | ALA A | 539 | 5169 | 5078 | 5245 | −3 | 85 | −37 | N |

| | | | | | | | | | | |
|---|---|---|---|---|---|---|---|---|---|---|
| ATOM | 1140 | CA | ALA A | 539 | 3.881 | 33.169 | 86.653 | 1.00 | 42.07 | C |

| | | | | | | | | | | | |
|---|---|---|---|---|---|---|---|---|---|---|---|
| ANISOU | 1140 | CA | ALA A | 539 | 5313 | 5294 | 5379 | −94 | 30 | −93 | C |

| | | | | | | | | | | |
|---|---|---|---|---|---|---|---|---|---|---|
| ATOM | 1141 | C | ALA A | 539 | 4.394 | 32.797 | 85.284 | 1.00 | 41.87 | C |

| | | | | | | | | | | | |
|---|---|---|---|---|---|---|---|---|---|---|---|
| ANISOU | 1141 | C | ALA A | 539 | 5126 | 5298 | 5486 | −154 | 184 | −81 | C |

| | | | | | | | | | | |
|---|---|---|---|---|---|---|---|---|---|---|
| ATOM | 1142 | O | ALA A | 539 | 3.795 | 31.916 | 84.631 | 1.00 | 42.02 | O |

| | | | | | | | | | | | |
|---|---|---|---|---|---|---|---|---|---|---|---|
| ANISOU | 1142 | O | ALA A | 539 | 4965 | 5298 | 5702 | −109 | 146 | −169 | O |

| | | | | | | | | | | |
|---|---|---|---|---|---|---|---|---|---|---|
| ATOM | 1143 | CB | ALA A | 539 | 2.715 | 34.130 | 86.525 | 1.00 | 42.83 | C |

| | | | | | | | | | | | |
|---|---|---|---|---|---|---|---|---|---|---|---|
| ANISOU | 1143 | CB | ALA A | 539 | 5446 | 5328 | 5500 | −33 | −41 | −28 | C |

| | | | | | | | | | | |
|---|---|---|---|---|---|---|---|---|---|---|
| ATOM | 1144 | N | MET A | 540 | 5.467 | 33.394 | 84.771 | 1.00 | 39.76 | N |

| | | | | | | | | | | | |
|---|---|---|---|---|---|---|---|---|---|---|---|
| ANISOU | 1144 | N | MET A | 540 | 4991 | 4816 | 5300 | −88 | 88 | −104 | N |

| | | | | | | | | | | |
|---|---|---|---|---|---|---|---|---|---|---|
| ATOM | 1145 | CA | MET A | 540 | 5.943 | 33.051 | 83.437 | 1.00 | 38.59 | C |

| | | | | | | | | | | | |
|---|---|---|---|---|---|---|---|---|---|---|---|
| ANISOU | 1145 | CA | MET A | 540 | 4789 | 4594 | 5278 | −57 | 91 | −65 | C |

| | | | | | | | | | | |
|---|---|---|---|---|---|---|---|---|---|---|
| ATOM | 1146 | C | MET A | 540 | 6.507 | 31.619 | 83.445 | 1.00 | 34.13 | C |

| | | | | | | | | | | | |
|---|---|---|---|---|---|---|---|---|---|---|---|
| ANISOU | 1146 | C | MET A | 540 | 3847 | 4429 | 4691 | −227 | 158 | −6 | C |

| | | | | | | | | | | |
|---|---|---|---|---|---|---|---|---|---|---|
| ATOM | 1147 | O | MET A | 540 | 6.464 | 30.884 | 82.468 | 1.00 | 33.31 | O |

| | | | | | | | | | | | |
|---|---|---|---|---|---|---|---|---|---|---|---|
| ANISOU | 1147 | O | MET A | 540 | 3620 | 4010 | 5025 | −131 | 272 | −88 | O |

| | | | | | | | | | | |
|---|---|---|---|---|---|---|---|---|---|---|
| ATOM | 1148 | CB | MET A | 540 | 7.086 | 33.962 | 82.977 | 1.00 | 41.08 | C |

| | | | | | | | | | | | |
|---|---|---|---|---|---|---|---|---|---|---|---|
| ANISOU | 1148 | CB | MET A | 540 | 4953 | 5188 | 5467 | −187 | 151 | 74 | C |

| | | | | | | | | | | |
|---|---|---|---|---|---|---|---|---|---|---|
| ATOM | 1149 | CG | MET A | 540 | 7.068 | 35.406 | 83.355 | 1.00 | 43.85 | C |

| | | | | | | | | | | | |
|---|---|---|---|---|---|---|---|---|---|---|---|
| ANISOU | 1149 | CG | MET A | 540 | 5580 | 5375 | 5705 | −6 | 20 | −53 | C |

| | | | | | | | | | | |
|---|---|---|---|---|---|---|---|---|---|---|
| ATOM | 1150 | SD | MET A | 540 | 5.603 | 36.403 | 83.157 | 1.00 | 46.11 | S |

| | | | | | | | | | | | |
|---|---|---|---|---|---|---|---|---|---|---|---|
| ANISOU | 1150 | SD | MET A | 540 | 5627 | 5748 | 6147 | 125 | −70 | −87 | S |

| | | | | | | | | | | |
|---|---|---|---|---|---|---|---|---|---|---|
| ATOM | 1151 | CE | MET A | 540 | 4.761 | 35.658 | 81.751 | 1.00 | 46.44 | C |

| | | | | | | | | | | | |
|---|---|---|---|---|---|---|---|---|---|---|---|
| ANISOU | 1151 | CE | MET A | 540 | 5900 | 5811 | 5933 | 78 | −64 | 33 | C |

| | | | | | | | | | | |
|---|---|---|---|---|---|---|---|---|---|---|
| ATOM | 1152 | N | SER A | 541 | 7.070 | 31.282 | 84.564 | 1.00 | 31.86 | N |

| | | | | | | | | | | | |
|---|---|---|---|---|---|---|---|---|---|---|---|
| ANISOU | 1152 | N | SER A | 541 | 3746 | 3817 | 4544 | −158 | 418 | 21 | N |

| | | | | | | | | | | |
|---|---|---|---|---|---|---|---|---|---|---|
| ATOM | 1153 | CA | SER A | 541 | 7.710 | 29.986 | 84.853 | 1.00 | 31.27 | C |

| | | | | | | | | | | | |
|---|---|---|---|---|---|---|---|---|---|---|---|
| ANISOU | 1153 | CA | SER A | 541 | 3578 | 3869 | 4433 | −112 | 282 | −17 | C |

| | | | | | | | | | | |
|---|---|---|---|---|---|---|---|---|---|---|
| ATOM | 1154 | C | SER A | 541 | 6.585 | 28.941 | 84.861 | 1.00 | 30.32 | C |

| | | | | | | | | | | | |
|---|---|---|---|---|---|---|---|---|---|---|---|
| ANISOU | 1154 | C | SER A | 541 | 3649 | 3616 | 4256 | −71 | 225 | −115 | C |

| | | | | | | | | | | |
|---|---|---|---|---|---|---|---|---|---|---|
| ATOM | 1155 | O | SER A | 541 | 6.751 | 27.800 | 84.465 | 1.00 | 27.86 | O |

| | | | | | | | | | | | |
|---|---|---|---|---|---|---|---|---|---|---|---|
| ANISOU | 1155 | O | SER A | 541 | 2924 | 3616 | 4046 | −18 | 555 | −162 | O |

| | | | | | | | | | | |
|---|---|---|---|---|---|---|---|---|---|---|
| ATOM | 1156 | CB | SER A | 541 | 8.515 | 30.046 | 86.103 | 1.00 | 33.71 | C |

| | | | | | | | | | | | |
|---|---|---|---|---|---|---|---|---|---|---|---|
| ANISOU | 1156 | CB | SER A | 541 | 4081 | 4255 | 4472 | −199 | 121 | −73 | C |

| | | | | | | | | | | |
|---|---|---|---|---|---|---|---|---|---|---|
| ATOM | 1157 | OG | SER A | 541 | 8.029 | 29.890 | 87.404 | 1.00 | 35.03 | O |

| | | | | | | | | | | | |
|---|---|---|---|---|---|---|---|---|---|---|---|
| ANISOU | 1157 | OG | SER A | 541 | 4054 | 4791 | 4464 | −143 | 144 | −374 | O |

| | | | | | | | | | | |
|---|---|---|---|---|---|---|---|---|---|---|
| ATOM | 1158 | N | PHE A | 542 | 5.397 | 29.413 | 85.315 | 1.00 | 28.90 | N |

| | | | | | | | | | | | |
|---|---|---|---|---|---|---|---|---|---|---|---|
| ANISOU | 1158 | N | PHE A | 542 | 3328 | 3510 | 4142 | −72 | 146 | −51 | N |

| | | | | | | | | | | |
|---|---|---|---|---|---|---|---|---|---|---|
| ATOM | 1159 | CA | PHE A | 542 | 4.279 | 28.441 | 85.248 | 1.00 | 30.40 | C |

| | | | | | | | | | | | |
|---|---|---|---|---|---|---|---|---|---|---|---|
| ANISOU | 1159 | CA | PHE A | 542 | 3529 | 3766 | 4258 | −158 | 163 | −100 | C |

| | | | | | | | | | | |
|---|---|---|---|---|---|---|---|---|---|---|
| ATOM | 1160 | C | PHE A | 542 | 3.792 | 28.145 | 83.865 | 1.00 | 28.18 | C |

| | | | | | | | | | | | |
|---|---|---|---|---|---|---|---|---|---|---|---|
| ANISOU | 1160 | C | PHE A | 542 | 3090 | 3480 | 4137 | −38 | 262 | −87 | C |

| | | | | | | | | | | |
|---|---|---|---|---|---|---|---|---|---|---|
| ATOM | 1161 | O | PHE A | 542 | 3.495 | 26.954 | 83.605 | 1.00 | 28.28 | O |

| | | | | | | | | | | | |
|---|---|---|---|---|---|---|---|---|---|---|---|
| ANISOU | 1161 | O | PHE A | 542 | 2991 | 3444 | 4308 | −17 | 294 | −186 | O |

| | | | | | | | | | | |
|---|---|---|---|---|---|---|---|---|---|---|
| ATOM | 1162 | CB | PHE A | 542 | 3.115 | 28.992 | 86.067 | 1.00 | 32.99 | C |

| | | | | | | | | | | | |
|---|---|---|---|---|---|---|---|---|---|---|---|
| ANISOU | 1162 | CB | PHE A | 542 | 3889 | 4161 | 4487 | −22 | 290 | −115 | C |

| | | | | | | | | | | |
|---|---|---|---|---|---|---|---|---|---|---|
| ATOM | 1163 | CG | PHE A | 542 | 3.352 | 28.591 | 87.485 | 1.00 | 35.58 | C |

| | | | | | | | | | | | |
|---|---|---|---|---|---|---|---|---|---|---|---|
| ANISOU | 1163 | CG | PHE A | 542 | 4335 | 4609 | 4575 | −27 | 50 | −3 | C |

| | | | | | | | | | | |
|---|---|---|---|---|---|---|---|---|---|---|
| ATOM | 1164 | CD1 | PHE A | 542 | 3.521 | 29.567 | 88.444 | 1.00 | 37.40 | C |

| | | | | | | | | | | | |
|---|---|---|---|---|---|---|---|---|---|---|---|
| ANISOU | 1164 | CD1 | PHE A | 542 | 4758 | 4693 | 4760 | −87 | 33 | −70 | C |

| | | | | | | | | | | |
|---|---|---|---|---|---|---|---|---|---|---|
| ATOM | 1165 | CD2 | PHE A | 542 | 3.359 | 27.251 | 87.839 | 1.00 | 37.35 | C |

| | | | | | | | | | | | |
|---|---|---|---|---|---|---|---|---|---|---|---|
| ANISOU | 1165 | CD2 | PHE A | 542 | 4793 | 4650 | 4749 | 22 | 48 | −16 | C |

| | | | | | | | | | | |
|---|---|---|---|---|---|---|---|---|---|---|
| ATOM | 1166 | CE1 | PHE A | 542 | 3.724 | 29.213 | 89.767 | 1.00 | 38.62 | C |

| | | | | | | | | | | | |
|---|---|---|---|---|---|---|---|---|---|---|---|
| ANISOU | 1166 | CE1 | PHE A | 542 | 4869 | 4968 | 4838 | 31 | −6 | 49 | C |

| | | | | | | | | | | |
|---|---|---|---|---|---|---|---|---|---|---|
| ATOM | 1167 | CE2 | PHE A | 542 | 3.576 | 26.892 | 89.156 | 1.00 | 38.49 | C |

| | | | | | | | | | | | |
|---|---|---|---|---|---|---|---|---|---|---|---|
| ANISOU | 1167 | CE2 | PHE A | 542 | 4888 | 4957 | 4780 | −50 | 10 | 15 | C |

| | | | | | | | | | | |
|---|---|---|---|---|---|---|---|---|---|---|
| ATOM | 1168 | CZ | PHE A | 542 | 3.739 | 27.878 | 90.110 | 1.00 | 38.82 | C |

| | | | | | | | | | | | |
|---|---|---|---|---|---|---|---|---|---|---|---|
| ANISOU | 1168 | CZ | PHE A | 542 | 4945 | 4898 | 4908 | −47 | 12 | −23 | C |

| | | | | | | | | | | |
|---|---|---|---|---|---|---|---|---|---|---|
| ATOM | 1169 | N | ALA A | 543 | 3.679 | 29.131 | 82.988 | 1.00 | 27.61 | N |

| | | | | | | | | | | | |
|---|---|---|---|---|---|---|---|---|---|---|---|
| ANISOU | 1169 | N | ALA A | 543 | 3063 | 3463 | 3966 | −24 | 176 | −153 | N |

| | | | | | | | | | | |
|---|---|---|---|---|---|---|---|---|---|---|
| ATOM | 1170 | CA | ALA A | 543 | 3.312 | 28.898 | 81.608 | 1.00 | 28.18 | C |

| | | | | | | | | | | | |
|---|---|---|---|---|---|---|---|---|---|---|---|
| ANISOU | 1170 | CA | ALA A | 543 | 3316 | 3458 | 3934 | 62 | 109 | −32 | C |

| | | | | | | | | | | |
|---|---|---|---|---|---|---|---|---|---|---|
| ATOM | 1171 | C | ALA A | 543 | 4.315 | 27.934 | 80.978 | 1.00 | 27.24 | C |

| | | | | | | | | | | | |
|---|---|---|---|---|---|---|---|---|---|---|---|
| ANISOU | 1171 | C | ALA A | 543 | 3100 | 3419 | 3832 | −39 | −43 | −41 | C |

| | | | | | | | | | | |
|---|---|---|---|---|---|---|---|---|---|---|
| ATOM | 1172 | O | ALA A | 543 | 3.891 | 27.090 | 80.197 | 1.00 | 27.41 | O |

| | | | | | | | | | | | |
|---|---|---|---|---|---|---|---|---|---|---|---|
| ANISOU | 1172 | O | ALA A | 543 | 3093 | 3300 | 4021 | −91 | 43 | −34 | O |

| | | | | | | | | | | |
|---|---|---|---|---|---|---|---|---|---|---|
| ATOM | 1173 | CB | ALA A | 543 | 3.207 | 30.162 | 80.775 | 1.00 | 30.56 | C |

| | | | | | | | | | | | |
|---|---|---|---|---|---|---|---|---|---|---|---|
| ANISOU | 1173 | CB | ALA A | 543 | 3874 | 3596 | 4141 | 47 | 28 | 63 | C |

| | | | | | | | | | | |
|---|---|---|---|---|---|---|---|---|---|---|
| ATOM | 1174 | N | ILE A | 544 | 5.619 | 28.080 | 81.245 | 1.00 | 24.40 | N |

| | | | | | | | | | | | |
|---|---|---|---|---|---|---|---|---|---|---|---|
| ANISOU | 1174 | N | ILE A | 544 | 2987 | 2720 | 3564 | −110 | 26 | 0 | N |

| | | | | | | | | | | |
|---|---|---|---|---|---|---|---|---|---|---|
| ATOM | 1175 | CA | ILE A | 544 | 6.597 | 27.158 | 80.714 | 1.00 | 23.25 | C |

| | | | | | | | | | | | |
|---|---|---|---|---|---|---|---|---|---|---|---|
| ANISOU | 1175 | CA | ILE A | 544 | 2809 | 2822 | 3203 | −109 | −13 | 83 | C |

| | | | | | | | | | | |
|---|---|---|---|---|---|---|---|---|---|---|
| ATOM | 1176 | C | ILE A | 544 | 6.320 | 25.756 | 81.267 | 1.00 | 21.89 | C |

| | | | | | | | | | | | |
|---|---|---|---|---|---|---|---|---|---|---|---|
| ANISOU | 1176 | C | ILE A | 544 | 2410 | 2778 | 3129 | −13 | 80 | 44 | C |

| | | | | | | | | | | |
|---|---|---|---|---|---|---|---|---|---|---|
| ATOM | 1177 | O | ILE A | 544 | 6.329 | 24.846 | 80.452 | 1.00 | 22.55 | O |

| | | | | | | | | | | | |
|---|---|---|---|---|---|---|---|---|---|---|---|
| ANISOU | 1177 | O | ILE A | 544 | 2457 | 2733 | 3378 | −308 | 59 | 30 | O |

| | | | | | | | | | | |
|---|---|---|---|---|---|---|---|---|---|---|
| ATOM | 1178 | CB | ILE A | 544 | 8.035 | 27.606 | 81.064 | 1.00 | 23.69 | C |

| | | | | | | | | | | | |
|---|---|---|---|---|---|---|---|---|---|---|---|
| ANISOU | 1178 | CB | ILE A | 544 | 2751 | 2967 | 3281 | −29 | 41 | 77 | C |

| | | | | | | | | | | |
|---|---|---|---|---|---|---|---|---|---|---|
| ATOM | 1179 | CG1 | ILE A | 544 | 8.336 | 28.899 | 80.280 | 1.00 | 24.17 | C |

| | | | | | | | | | | | |
|---|---|---|---|---|---|---|---|---|---|---|---|
| ANISOU | 1179 | CG1 | ILE A | 544 | 2814 | 2955 | 3412 | −190 | −99 | 71 | C |

| | | | | | | | | | | |
|---|---|---|---|---|---|---|---|---|---|---|
| ATOM | 1180 | CG2 | ILE A | 544 | 9.080 | 26.559 | 80.732 | 1.00 | 23.76 | C |

| | | | | | | | | | | | |
|---|---|---|---|---|---|---|---|---|---|---|---|
| ANISOU | 1180 | CG2 | ILE A | 544 | 2973 | 2883 | 3173 | 0 | −53 | −30 | C |

| | | | | | | | | | | |
|---|---|---|---|---|---|---|---|---|---|---|
| ATOM | 1181 | CD1 | ILE A | 544 | 9.606 | 29.597 | 80.759 | 1.00 | 24.04 | C |

| | | | | | | | | | | | |
|---|---|---|---|---|---|---|---|---|---|---|---|
| ANISOU | 1181 | CD1 | ILE A | 544 | 2749 | 2965 | 3420 | −194 | 53 | 92 | C |

| | | | | | | | | | | |
|---|---|---|---|---|---|---|---|---|---|---|
| ATOM | 1182 | N | LEU A | 545 | 6.202 | 25.602 | 82.589 | 1.00 | 23.24 | N |

| | | | | | | | | | | | |
|---|---|---|---|---|---|---|---|---|---|---|---|
| ANISOU | 1182 | N | LEU A | 545 | 2815 | 2786 | 3229 | −90 | 47 | 105 | N |

| | | | | | | | | | | |
|---|---|---|---|---|---|---|---|---|---|---|
| ATOM | 1183 | CA | LEU A | 545 | 5.971 | 24.239 | 83.093 | 1.00 | 22.70 | C |

| | | | | | | | | | | | |
|---|---|---|---|---|---|---|---|---|---|---|---|
| ANISOU | 1183 | CA | LEU A | 545 | 2755 | 2763 | 3107 | 83 | 147 | 97 | C |

| | | | | | | | | | | |
|---|---|---|---|---|---|---|---|---|---|---|
| ATOM | 1184 | C | LEU A | 545 | 4.719 | 23.633 | 82.517 | 1.00 | 23.03 | C |

| | | | | | | | | | | | |
|---|---|---|---|---|---|---|---|---|---|---|---|
| ANISOU | 1184 | C | LEU A | 545 | 2696 | 2820 | 3235 | 55 | 137 | 38 | C |

| | | | | | | | | | | |
|---|---|---|---|---|---|---|---|---|---|---|
| ATOM | 1185 | O | LEU A | 545 | 4.740 | 22.456 | 82.099 | 1.00 | 22.91 | O |

| | | | | | | | | | | | |
|---|---|---|---|---|---|---|---|---|---|---|---|
| ANISOU | 1185 | O | LEU A | 545 | 2514 | 2725 | 3464 | −50 | 277 | 67 | O |

| | | | | | | | | | | |
|---|---|---|---|---|---|---|---|---|---|---|
| ATOM | 1186 | CB | LEU A | 545 | 5.975 | 24.256 | 84.626 | 1.00 | 23.22 | C |

| | | | | | | | | | | | |
|---|---|---|---|---|---|---|---|---|---|---|---|
| ANISOU | 1186 | CB | LEU A | 545 | 2832 | 2865 | 3127 | 17 | −2 | −49 | C |

| | | | | | | | | | | |
|---|---|---|---|---|---|---|---|---|---|---|
| ATOM | 1187 | CG | LEU A | 545 | 7.316 | 24.654 | 85.247 | 1.00 | 23.44 | C |

| | | | | | | | | | | | |
|---|---|---|---|---|---|---|---|---|---|---|---|
| ANISOU | 1187 | CG | LEU A | 545 | 2968 | 2825 | 3114 | −155 | 27 | −19 | C |

| | | | | | | | | | | |
|---|---|---|---|---|---|---|---|---|---|---|
| ATOM | 1188 | CD1 | LEU A | 545 | 7.091 | 25.039 | 86.694 | 1.00 | 25.32 | C |

| | | | | | | | | | | | |
|---|---|---|---|---|---|---|---|---|---|---|---|
| ANISOU | 1188 | CD1 | LEU A | 545 | 3398 | 3041 | 3181 | −46 | 89 | 24 | C |

| | | | | | | | | | | |
|---|---|---|---|---|---|---|---|---|---|---|
| ATOM | 1189 | CD2 | LEU A | 545 | 8.294 | 23.497 | 85.144 | 1.00 | 23.99 | C |

| | | | | | | | | | | | |
|---|---|---|---|---|---|---|---|---|---|---|---|
| ANISOU | 1189 | CD2 | LEU A | 545 | 3040 | 2933 | 3144 | −124 | 71 | −113 | C |

| | | | | | | | | | | |
|---|---|---|---|---|---|---|---|---|---|---|
| ATOM | 1190 | N | THR A | 546 | 3.585 | 24.400 | 82.423 | 1.00 | 23.28 | N |

| | | | | | | | | | | | |
|---|---|---|---|---|---|---|---|---|---|---|---|
| ANISOU | 1190 | N | THR A | 546 | 2713 | 2810 | 3322 | 57 | 151 | 281 | N |

| | | | | | | | | | | |
|---|---|---|---|---|---|---|---|---|---|---|
| ATOM | 1191 | CA | THR A | 546 | 2.410 | 23.752 | 81.835 | 1.00 | 23.36 | C |

| | | | | | | | | | | | |
|---|---|---|---|---|---|---|---|---|---|---|---|
| ANISOU | 1191 | CA | THR A | 546 | 2805 | 2868 | 3203 | 107 | 153 | 104 | C |

| | | | | | | | | | | |
|---|---|---|---|---|---|---|---|---|---|---|
| ATOM | 1192 | C | THR A | 546 | 2.547 | 23.433 | 80.360 | 1.00 | 23.93 | C |

| | | | | | | | | | | | |
|---|---|---|---|---|---|---|---|---|---|---|---|
| ANISOU | 1192 | C | THR A | 546 | 2851 | 2892 | 3350 | 101 | 219 | 74 | C |

| | | | | | | | | | | |
|---|---|---|---|---|---|---|---|---|---|---|
| ATOM | 1193 | O | THR A | 546 | 2.063 | 22.396 | 79.881 | 1.00 | 25.26 | O |

| | | | | | | | | | | | |
|---|---|---|---|---|---|---|---|---|---|---|---|
| ANISOU | 1193 | O | THR A | 546 | 2518 | 3265 | 3816 | −11 | 308 | −239 | O |

| | | | | | | | | | | |
|---|---|---|---|---|---|---|---|---|---|---|
| ATOM | 1194 | CB | THR A | 546 | 1.134 | 24.602 | 82.122 | 1.00 | 23.80 | C |

| | | | | | | | | | | | |
|---|---|---|---|---|---|---|---|---|---|---|---|
| ANISOU | 1194 | CB | THR A | 546 | 2923 | 2874 | 3245 | 272 | 152 | 110 | C |

| | | | | | | | | | | |
|---|---|---|---|---|---|---|---|---|---|---|
| ATOM | 1195 | OG1 | THR A | 546 | 1.275 | 25.892 | 81.529 | 1.00 | 25.40 | O |

| | | | | | | | | | | | |
|---|---|---|---|---|---|---|---|---|---|---|---|
| ANISOU | 1195 | OG1 | THR A | 546 | 3111 | 3131 | 3409 | 81 | 287 | 289 | O |

| | | | | | | | | | | |
|---|---|---|---|---|---|---|---|---|---|---|
| ATOM | 1196 | CG2 | THR A | 546 | 1.013 | 24.808 | 83.628 | 1.00 | 25.03 | C |

| | | | | | | | | | | | |
|---|---|---|---|---|---|---|---|---|---|---|---|
| ANISOU | 1196 | CG2 | THR A | 546 | 3109 | 3137 | 3263 | 163 | 215 | 23 | C |

| | | | | | | | | | | |
|---|---|---|---|---|---|---|---|---|---|---|
| ATOM | 1197 | N | ASP A | 547 | 3.336 | 24.193 | 79.609 | 1.00 | 23.36 | N |

| | | | | | | | | | | | |
|---|---|---|---|---|---|---|---|---|---|---|---|
| ANISOU | 1197 | N | ASP A | 547 | 2731 | 2860 | 3284 | 12 | 198 | 24 | N |

| | | | | | | | | | | |
|---|---|---|---|---|---|---|---|---|---|---|
| ATOM | 1198 | CA | ASP A | 547 | 3.586 | 23.924 | 78.212 | 1.00 | 22.59 | C |

| | | | | | | | | | | | |
|---|---|---|---|---|---|---|---|---|---|---|---|
| ANISOU | 1199 | CA | ASP A | 547 | 2640 | 2728 | 3216 | 17 | 159 | 71 | C |

| | | | | | | | | | | |
|---|---|---|---|---|---|---|---|---|---|---|
| ATOM | 1199 | C | ASP A | 547 | 4.348 | 22.589 | 78.092 | 1.00 | 22.24 | C |

| | | | | | | | | | | | |
|---|---|---|---|---|---|---|---|---|---|---|---|
| ANISOU | 1199 | C | ASP A | 547 | 2607 | 2709 | 3134 | −49 | 194 | 47 | C |

| | | | | | | | | | | |
|---|---|---|---|---|---|---|---|---|---|---|
| ATOM | 1200 | O | ASP A | 547 | 3.981 | 21.843 | 77.203 | 1.00 | 23.03 | O |

| | | | | | | | | | | | |
|---|---|---|---|---|---|---|---|---|---|---|---|
| ANISOU | 1200 | O | ASP A | 547 | 2636 | 2636 | 3479 | −243 | 321 | 67 | O |

| | | | | | | | | | | |
|---|---|---|---|---|---|---|---|---|---|---|
| ATOM | 1201 | CB | ASP A | 547 | 4.468 | 25.026 | 77.571 | 1.00 | 24.02 | C |

| | | | | | | | | | | | |
|---|---|---|---|---|---|---|---|---|---|---|---|
| ANISOU | 1201 | CB | ASP A | 547 | 2977 | 2889 | 3261 | −129 | 98 | 85 | C |

| | | | | | | | | | | |
|---|---|---|---|---|---|---|---|---|---|---|
| ATOM | 1202 | CG | ASP A | 547 | 3.654 | 26.182 | 77.017 | 1.00 | 25.48 | C |

| | | | | | | | | | | | |
|---|---|---|---|---|---|---|---|---|---|---|---|
| ANISOU | 1202 | CG | ASP A | 547 | 2948 | 3175 | 3557 | 21 | 12 | 187 | C |

| | | | | | | | | | | |
|---|---|---|---|---|---|---|---|---|---|---|
| ATOM | 1203 | OD1 | ASP A | 547 | 2.412 | 26.058 | 77.030 | 1.00 | 27.64 | O |

| | | | | | | | | | | | |
|---|---|---|---|---|---|---|---|---|---|---|---|
| ANISOU | 1203 | OD1 | ASP A | 547 | 3264 | 3496 | 3741 | −106 | −39 | 263 | O |

| | | | | | | | | | | |
|---|---|---|---|---|---|---|---|---|---|---|
| ATOM | 1204 | OD2 | ASP A | 547 | 4.222 | 27.221 | 76.576 | 1.00 | 24.33 | O |

| | | | | | | | | | | | |
|---|---|---|---|---|---|---|---|---|---|---|---|
| ANISOU | 1204 | OD2 | ASP A | 547 | 2766 | 3161 | 3318 | 223 | 132 | 261 | O |

| | | | | | | | | | | |
|---|---|---|---|---|---|---|---|---|---|---|
| ATOM | 1205 | N | LEU A | 548 | 5.324 | 22.377 | 78.963 | 1.00 | 22.87 | N |

| | | | | | | | | | | | |
|---|---|---|---|---|---|---|---|---|---|---|---|
| ANISOU | 1205 | N | LEU A | 548 | 2872 | 2491 | 3328 | −61 | 256 | 47 | N |

| | | | | | | | | | | |
|---|---|---|---|---|---|---|---|---|---|---|
| ATOM | 1206 | CA | LEU A | 548 | 6.120 | 21.147 | 78.866 | 1.00 | 21.74 | C |

| | | | | | | | | | | | |
|---|---|---|---|---|---|---|---|---|---|---|---|
| ANISOU | 1206 | CA | LEU A | 548 | 2446 | 2575 | 3240 | −37 | 117 | 130 | C |

| | | | | | | | | | | |
|---|---|---|---|---|---|---|---|---|---|---|
| ATOM | 1207 | C | LEU A | 548 | 5.230 | 19.963 | 79.288 | 1.00 | 22.05 | C |

| | | | | | | | | | | | |
|---|---|---|---|---|---|---|---|---|---|---|---|
| ANISOU | 1207 | C | LEU A | 548 | 2709 | 2620 | 3048 | −161 | 154 | 51 | C |

| | | | | | | | | | | |
|---|---|---|---|---|---|---|---|---|---|---|
| ATOM | 1208 | O | LEU A | 548 | 5.381 | 18.895 | 78.712 | 1.00 | 20.79 | O |

| | | | | | | | | | | | |
|---|---|---|---|---|---|---|---|---|---|---|---|
| ANISOU | 1208 | O | LEU A | 548 | 2505 | 2472 | 2922 | −377 | 267 | 123 | O |

| | | | | | | | | | | |
|---|---|---|---|---|---|---|---|---|---|---|
| ATOM | 1209 | CB | LEU A | 548 | 7.401 | 21.241 | 79.689 | 1.00 | 22.72 | C |

| | | | | | | | | | | | |
|---|---|---|---|---|---|---|---|---|---|---|---|
| ANISOU | 1209 | CB | LEU A | 548 | 2922 | 2632 | 3081 | −84 | 26 | 68 | C |

| | | | | | | | | | | |
|---|---|---|---|---|---|---|---|---|---|---|
| ATOM | 1210 | CG | LEU A | 548 | 8.365 | 22.349 | 79.186 | 1.00 | 23.16 | C |

| | | | | | | | | | | | |
|---|---|---|---|---|---|---|---|---|---|---|---|
| ANISOU | 1210 | CG | LEU A | 548 | 2879 | 2809 | 3111 | −138 | 77 | 100 | C |

| | | | | | | | | | | |
|---|---|---|---|---|---|---|---|---|---|---|
| ATOM | 1211 | CD1 | LEU A | 548 | 9.459 | 22.500 | 80.226 | 1.00 | 23.26 | C |

| | | | | | | | | | | | |
|---|---|---|---|---|---|---|---|---|---|---|---|
| ANISOU | 1211 | CD1 | LEU A | 548 | 2610 | 3008 | 3220 | −185 | 57 | 106 | C |

| | | | | | | | | | | |
|---|---|---|---|---|---|---|---|---|---|---|
| ATOM | 1212 | CD2 | LEU A | 548 | 8.905 | 21.969 | 77.810 | 1.00 | 23.43 | C |

| | | | | | | | | | | | |
|---|---|---|---|---|---|---|---|---|---|---|---|
| ANISOU | 1212 | CD2 | LEU A | 548 | 2988 | 2840 | 3076 | −75 | 94 | 160 | C |

| | | | | | | | | | | |
|---|---|---|---|---|---|---|---|---|---|---|
| ATOM | 1213 | N | GLN A | 549 | 4.357 | 20.172 | 80.279 | 1.00 | 22.85 | N |

| | | | | | | | | | | | |
|---|---|---|---|---|---|---|---|---|---|---|---|
| ANISOU | 1213 | N | GLN A | 549 | 2718 | 2776 | 3187 | −245 | 176 | −77 | N |

| | | | | | | | | | | |
|---|---|---|---|---|---|---|---|---|---|---|
| ATOM | 1214 | CA | GLN A | 549 | 3.462 | 19.079 | 80.712 | 1.00 | 24.15 | C |

| | | | | | | | | | | | |
|---|---|---|---|---|---|---|---|---|---|---|---|
| ANISOU | 1214 | CA | GLN A | 549 | 2874 | 2976 | 3324 | −284 | 269 | 74 | C |

| | | | | | | | | | | |
|---|---|---|---|---|---|---|---|---|---|---|
| ATOM | 1215 | C | GLN A | 549 | 2.494 | 18.733 | 79.598 | 1.00 | 24.61 | C |

| | | | | | | | | | | | |
|---|---|---|---|---|---|---|---|---|---|---|---|
| ANISOU | 1215 | C | GLN A | 549 | 3038 | 3071 | 3241 | −118 | 217 | 51 | C |

| | | | | | | | | | | |
|---|---|---|---|---|---|---|---|---|---|---|
| ATOM | 1216 | O | GLN A | 549 | 2.283 | 17.554 | 79.279 | 1.00 | 24.12 | O |

| | | | | | | | | | | | |
|---|---|---|---|---|---|---|---|---|---|---|---|
| ANISOU | 1216 | O | GLN A | 549 | 2527 | 3132 | 3508 | −389 | 233 | 175 | O |

| | | | | | | | | | | |
|---|---|---|---|---|---|---|---|---|---|---|
| ATOM | 1217 | CB | GLN A | 549 | 2.712 | 19.459 | 82.007 | 1.00 | 25.04 | C |

| | | | | | | | | | | | |
|---|---|---|---|---|---|---|---|---|---|---|---|
| ANISOU | 1217 | CB | GLN A | 549 | 2996 | 3091 | 3425 | −259 | 296 | −52 | C |

| | | | | | | | | | | |
|---|---|---|---|---|---|---|---|---|---|---|
| ATOM | 1218 | CG | GLN A | 549 | 1.749 | 18.311 | 82.410 | 1.00 | 26.70 | C |

| | | | | | | | | | | | |
|---|---|---|---|---|---|---|---|---|---|---|---|
| ANISOU | 1218 | CG | GLN A | 549 | 3371 | 3092 | 3684 | −292 | 275 | 64 | C |

| | | | | | | | | | | |
|---|---|---|---|---|---|---|---|---|---|---|
| ATOM | 1219 | CD | GLN A | 549 | 1.112 | 18.676 | 83.738 | 1.00 | 28.24 | C |

| | | | | | | | | | | | |
|---|---|---|---|---|---|---|---|---|---|---|---|
| ANISOU | 1219 | CD | GLN A | 549 | 3323 | 3531 | 3875 | −84 | 361 | 61 | C |

| | | | | | | | | | | |
|---|---|---|---|---|---|---|---|---|---|---|
| ATOM | 1220 | OE1 | GLN A | 549 | 0.731 | 19.849 | 83.875 | 1.00 | 28.70 | O |

| | | | | | | | | | | | |
|---|---|---|---|---|---|---|---|---|---|---|---|
| ANISOU | 1220 | OE1 | GLN A | 549 | 3378 | 3418 | 4109 | −194 | 636 | 61 | O |

| | | | | | | | | | | |
|---|---|---|---|---|---|---|---|---|---|---|
| ATOM | 1221 | NE2 | GLN A | 549 | 1.067 | 17.723 | 84.652 | 1.00 | 28.03 | N |

| | | | | | | | | | | | |
|---|---|---|---|---|---|---|---|---|---|---|---|
| ANISOU | 1221 | NE2 | GLN A | 549 | 3248 | 3564 | 3836 | −151 | 289 | 70 | N |

| | | | | | | | | | | |
|---|---|---|---|---|---|---|---|---|---|---|
| ATOM | 1222 | N | LYS A | 550 | 2.023 | 19.773 | 78.896 | 1.00 | 25.30 | N |

| | | | | | | | | | | | |
|---|---|---|---|---|---|---|---|---|---|---|---|
| ANISOU | 1222 | N | LYS A | 550 | 2979 | 3168 | 3468 | −90 | 108 | 139 | N |

| | | | | | | | | | | |
|---|---|---|---|---|---|---|---|---|---|---|
| ATOM | 1223 | CA | LYS A | 550 | 1.165 | 19.575 | 77.740 | 1.00 | 25.70 | C |

| | | | | | | | | | | | |
|---|---|---|---|---|---|---|---|---|---|---|---|
| ANISOU | 1223 | CA | LYS A | 550 | 3203 | 3128 | 3434 | −228 | 126 | 145 | C |

| | | | | | | | | | | |
|---|---|---|---|---|---|---|---|---|---|---|
| ATOM | 1224 | C | LYS A | 550 | 1.855 | 18.767 | 76.673 | 1.00 | 24.53 | C |

| | | | | | | | | | | | |
|---|---|---|---|---|---|---|---|---|---|---|---|
| ANISOU | 1224 | C | LYS A | 550 | 2856 | 3036 | 3429 | −262 | 78 | 152 | C |

| | | | | | | | | | | |
|---|---|---|---|---|---|---|---|---|---|---|
| ATOM | 1225 | O | LYS A | 550 | 1.333 | 17.855 | 76.045 | 1.00 | 23.16 | O |

| | | | | | | | | | | | |
|---|---|---|---|---|---|---|---|---|---|---|---|
| ANISOU | 1225 | O | LYS A | 550 | 2326 | 2988 | 3486 | −472 | 81 | 361 | O |

| | | | | | | | | | | |
|---|---|---|---|---|---|---|---|---|---|---|
| ATOM | 1226 | CB | LYS A | 550 | 0.712 | 20.937 | 77.181 | 1.00 | 28.22 | C |

| | | | | | | | | | | | |
|---|---|---|---|---|---|---|---|---|---|---|---|
| ANISOU | 1226 | CB | LYS A | 550 | 3592 | 3329 | 3799 | −16 | −29 | 166 | C |

| | | | | | | | | | | |
|---|---|---|---|---|---|---|---|---|---|---|
| ATOM | 1227 | CG | LYS A | 550 | −0.155 | 20.713 | 75.941 | 1.00 | 31.76 | C |

| | | | | | | | | | | | |
|---|---|---|---|---|---|---|---|---|---|---|---|
| ANISOU | 1227 | CG | LYS A | 550 | 4057 | 4034 | 3977 | −202 | −152 | −12 | C |

| | | | | | | | | | | |
|---|---|---|---|---|---|---|---|---|---|---|
| ATOM | 1228 | CD | LYS A | 550 | −0.798 | 22.002 | 75.495 | 1.00 | 34.48 | C |

| | | | | | | | | | | | |
|---|---|---|---|---|---|---|---|---|---|---|---|
| ANISOU | 1228 | CD | LYS A | 550 | 4395 | 4240 | 4465 | −27 | −84 | 67 | C |

| | | | | | | | | | | |
|---|---|---|---|---|---|---|---|---|---|---|
| ATOM | 1229 | CE | LYS A | 550 | 0.218 | 23.051 | 75.036 | 1.00 | 36.00 | C |

| | | | | | | | | | | | |
|---|---|---|---|---|---|---|---|---|---|---|---|
| ANISOU | 1229 | CE | LYS A | 550 | 4448 | 4437 | 4793 | −124 | −73 | 91 | C |

| | | | | | | | | | | |
|---|---|---|---|---|---|---|---|---|---|---|
| ATOM | 1230 | NZ | LYS A | 550 | −0.506 | 24.249 | 74.503 | 1.00 | 36.21 | N |

| | | | | | | | | | | | |
|---|---|---|---|---|---|---|---|---|---|---|---|
| ANISOU | 1230 | NZ | LYS A | 550 | 4398 | 4531 | 4830 | −109 | −120 | 125 | N |

| | | | | | | | | | | |
|---|---|---|---|---|---|---|---|---|---|---|
| ATOM | 1231 | N | TYR A | 551 | 3.145 | 19.062 | 76.396 | 1.00 | 22.08 | N |

| | | | | | | | | | | | |
|---|---|---|---|---|---|---|---|---|---|---|---|
| ANISOU | 1231 | N | TYR A | 551 | 2503 | 2664 | 3225 | −274 | 86 | 195 | N |

| | | | | | | | | | | |
|---|---|---|---|---|---|---|---|---|---|---|
| ATOM | 1232 | CA | TYR A | 551 | 3.909 | 18.345 | 75.403 | 1.00 | 20.84 | C |

| | | | | | | | | | | | |
|---|---|---|---|---|---|---|---|---|---|---|---|
| ANISOU | 1232 | CA | TYR A | 551 | 2400 | 2545 | 2972 | −330 | −107 | 215 | C |

| | | | | | | | | | | |
|---|---|---|---|---|---|---|---|---|---|---|
| ATOM | 1233 | C | TYR A | 551 | 3.939 | 16.846 | 75.739 | 1.00 | 22.16 | C |

| | | | | | | | | | | | |
|---|---|---|---|---|---|---|---|---|---|---|---|
| ANISOU | 1233 | C | TYR A | 551 | 2678 | 2630 | 3111 | −121 | −82 | 136 | C |

| | | | | | | | | | | |
|---|---|---|---|---|---|---|---|---|---|---|
| ATOM | 1234 | O | TYR A | 551 | 3.842 | 16.000 | 74.871 | 1.00 | 23.19 | O |

| | | | | | | | | | | | |
|---|---|---|---|---|---|---|---|---|---|---|---|
| ANISOU | 1234 | O | TYR A | 551 | 2812 | 2901 | 3100 | −254 | −184 | 114 | O |

| | | | | | | | | | | |
|---|---|---|---|---|---|---|---|---|---|---|
| ATOM | 1235 | CB | TYR A | 551 | 5.375 | 18.882 | 75.293 | 1.00 | 19.68 | C |

| | | | | | | | | | | | |
|---|---|---|---|---|---|---|---|---|---|---|---|
| ANISOU | 1235 | CB | TYR A | 551 | 2346 | 2340 | 2791 | −357 | −31 | 87 | C |

| | | | | | | | | | | |
|---|---|---|---|---|---|---|---|---|---|---|
| ATOM | 1236 | CG | TYR A | 551 | 6.163 | 18.079 | 74.261 | 1.00 | 19.76 | C |

| | | | | | | | | | | | |
|---|---|---|---|---|---|---|---|---|---|---|---|
| ANISOU | 1236 | CG | TYR A | 551 | 2121 | 2480 | 2908 | −346 | 16 | 116 | C |

| | | | | | | | | | | |
|---|---|---|---|---|---|---|---|---|---|---|
| ATOM | 1237 | CD1 | TYR A | 551 | 5.966 | 18.245 | 72.892 | 1.00 | 19.83 | C |

| | | | | | | | | | | | |
|---|---|---|---|---|---|---|---|---|---|---|---|
| ANISOU | 1237 | CD1 | TYR A | 551 | 2216 | 2489 | 2828 | −431 | 36 | −34 | C |

| | | | | | | | | | | |
|---|---|---|---|---|---|---|---|---|---|---|
| ATOM | 1238 | CD2 | TYR A | 551 | 7.059 | 17.110 | 74.685 | 1.00 | 20.99 | C |

| | | | | | | | | | | | |
|---|---|---|---|---|---|---|---|---|---|---|---|
| ANISOU | 1238 | CD2 | TYR A | 551 | 2389 | 2410 | 3165 | −388 | 45 | 41 | C |

| | | | | | | | | | | |
|---|---|---|---|---|---|---|---|---|---|---|
| ATOM | 1239 | CE1 | TYR A | 551 | 6.647 | 17.464 | 71.979 | 1.00 | 19.92 | C |

| | | | | | | | | | | | |
|---|---|---|---|---|---|---|---|---|---|---|---|
| ANISOU | 1239 | CE1 | TYR A | 551 | 2278 | 2447 | 2845 | −361 | −42 | −31 | C |

| | | | | | | | | | | |
|---|---|---|---|---|---|---|---|---|---|---|
| ATOM | 1240 | CE2 | TYR A | 551 | 7.770 | 16.333 | 73.799 | 1.00 | 19.81 | C |

| | | | | | | | | | | | |
|---|---|---|---|---|---|---|---|---|---|---|---|
| ANISOU | 1240 | CE2 | TYR A | 551 | 2230 | 2390 | 2907 | −358 | −83 | −65 | C |

| | | | | | | | | | | |
|---|---|---|---|---|---|---|---|---|---|---|
| ATOM | 1241 | CZ | TYR A | 551 | 7.554 | 16.512 | 72.449 | 1.00 | 20.68 | C |

| | | | | | | | | | | | |
|---|---|---|---|---|---|---|---|---|---|---|---|
| ANISOU | 1241 | CZ | TYR A | 551 | 2485 | 2446 | 2925 | −321 | −18 | 79 | C |

| | | | | | | | | | | |
|---|---|---|---|---|---|---|---|---|---|---|
| ATOM | 1242 | OH | TYR A | 551 | 8.268 | 15.769 | 71.555 | 1.00 | 21.83 | O |

| | | | | | | | | | | | |
|---|---|---|---|---|---|---|---|---|---|---|---|
| ANISOU | 1242 | OH | TYR A | 551 | 2357 | 2564 | 3373 | −132 | −156 | −63 | O |

| | | | | | | | | | | |
|---|---|---|---|---|---|---|---|---|---|---|
| ATOM | 1243 | N | VAL A | 552 | 4.265 | 16.533 | 76.986 | 1.00 | 21.72 | N |

| | | | | | | | | | | | |
|---|---|---|---|---|---|---|---|---|---|---|---|
| ANISOU | 1243 | N | VAL A | 552 | 2386 | 2787 | 3078 | −120 | −30 | 180 | N |

| | | | | | | | | | | |
|---|---|---|---|---|---|---|---|---|---|---|
| ATOM | 1244 | CA | VAL A | 552 | 4.385 | 15.135 | 77.406 | 1.00 | 22.67 | C |

| | | | | | | | | | | | |
|---|---|---|---|---|---|---|---|---|---|---|---|
| ANISOU | 1244 | CA | VAL A | 552 | 2549 | 2802 | 3262 | −149 | −13 | 132 | C |

| | | | | | | | | | | |
|---|---|---|---|---|---|---|---|---|---|---|
| ATOM | 1245 | C | VAL A | 552 | 3.043 | 14.392 | 77.218 | 1.00 | 23.51 | C |

| | | | | | | | | | | | |
|---|---|---|---|---|---|---|---|---|---|---|---|
| ANISOU | 1245 | C | VAL A | 552 | 2671 | 2936 | 3325 | −258 | −5 | 62 | C |

| | | | | | | | | | | |
|---|---|---|---|---|---|---|---|---|---|---|
| ATOM | 1246 | O | VAL A | 552 | 3.042 | 13.279 | 76.656 | 1.00 | 24.33 | O |

| | | | | | | | | | | | |
|---|---|---|---|---|---|---|---|---|---|---|---|
| ANISOU | 1246 | O | VAL A | 552 | 2665 | 3018 | 3563 | −221 | −92 | 32 | O |

| | | | | | | | | | | |
|---|---|---|---|---|---|---|---|---|---|---|
| ATOM | 1247 | CB | VAL A | 552 | 4.835 | 15.022 | 78.856 | 1.00 | 22.88 | C |

| | | | | | | | | | | | |
|---|---|---|---|---|---|---|---|---|---|---|---|
| ANISOU | 1247 | CB | VAL A | 552 | 2728 | 2733 | 3233 | −267 | −1 | −12 | C |

| | | | | | | | | | | |
|---|---|---|---|---|---|---|---|---|---|---|
| ATOM | 1248 | CG1 | VAL A | 552 | 4.887 | 13.538 | 79.270 | 1.00 | 23.22 | C |

| | | | | | | | | | | | |
|---|---|---|---|---|---|---|---|---|---|---|---|
| ANISOU | 1248 | CG1 | VAL A | 552 | 2805 | 2751 | 3266 | −108 | −86 | 143 | C |

| | | | | | | | | | | |
|---|---|---|---|---|---|---|---|---|---|---|
| ATOM | 1249 | CG2 | VAL A | 552 | 6.250 | 15.553 | 79.032 | 1.00 | 21.76 | C |

| | | | | | | | | | | | |
|---|---|---|---|---|---|---|---|---|---|---|---|
| ANISOU | 1249 | CG2 | VAL A | 552 | 2469 | 2657 | 3142 | −30 | −118 | 65 | C |

| | | | | | | | | | | |
|---|---|---|---|---|---|---|---|---|---|---|
| ATOM | 1250 | N | MET A | 553 | 1.989 | 15.073 | 77.663 | 1.00 | 23.62 | N |

| | | | | | | | | | | | |
|---|---|---|---|---|---|---|---|---|---|---|---|
| ANISOU | 1250 | N | MET A | 553 | 2680 | 2877 | 3407 | −202 | −72 | 195 | N |

| | | | | | | | | | | |
|---|---|---|---|---|---|---|---|---|---|---|
| ATOM | 1251 | CA | MET A | 553 | 0.652 | 14.423 | 77.505 | 1.00 | 24.68 | C |

| | | | | | | | | | | | |
|---|---|---|---|---|---|---|---|---|---|---|---|
| ANISOU | 1251 | CA | MET A | 553 | 2803 | 2951 | 3624 | −329 | −27 | 129 | C |

| | | | | | | | | | | |
|---|---|---|---|---|---|---|---|---|---|---|
| ATOM | 1252 | C | MET A | 553 | 0.296 | 14.201 | 76.061 | 1.00 | 26.17 | C |

| | | | | | | | | | | | |
|---|---|---|---|---|---|---|---|---|---|---|---|
| ANISOU | 1252 | C | MET A | 553 | 3177 | 3123 | 3644 | −57 | −15 | 61 | C |

| | | | | | | | | | | |
|---|---|---|---|---|---|---|---|---|---|---|
| ATOM | 1253 | O | MET A | 553 | −0.177 | 13.097 | 75.668 | 1.00 | 28.15 | O |

| | | | | | | | | | | | |
|---|---|---|---|---|---|---|---|---|---|---|---|
| ANISOU | 1253 | O | MET A | 553 | 3440 | 3305 | 3950 | −389 | −109 | 53 | O |

| | | | | | | | | | | |
|---|---|---|---|---|---|---|---|---|---|---|
| ATOM | 1254 | CB | MET A | 553 | −0.353 | 15.281 | 78.272 | 1.00 | 23.70 | C |

| | | | | | | | | | | | |
|---|---|---|---|---|---|---|---|---|---|---|---|
| ANISOU | 1254 | CB | MET A | 553 | 2706 | 2827 | 3473 | −467 | −1 | 177 | C |

| | | | | | | | | | | |
|---|---|---|---|---|---|---|---|---|---|---|
| ATOM | 1255 | CG | MET A | 553 | −0.220 | 15.213 | 79.787 | 1.00 | 26.39 | C |

| | | | | | | | | | | | |
|---|---|---|---|---|---|---|---|---|---|---|---|
| ANISOU | 1255 | CG | MET A | 553 | 3285 | 3188 | 3553 | −376 | 45 | 88 | C |

| | | | | | | | | | | |
|---|---|---|---|---|---|---|---|---|---|---|
| ATOM | 1256 | SD | MET A | 553 | −1.312 | 16.315 | 80.696 | 1.00 | 29.71 | 5 |

| | | | | | | | | | | | |
|---|---|---|---|---|---|---|---|---|---|---|---|
| ANISOU | 1256 | SD | MET A | 553 | 3253 | 3855 | 4178 | −231 | 342 | 92 | 5 |

| | | | | | | | | | | |
|---|---|---|---|---|---|---|---|---|---|---|
| ATOM | 1257 | CE | MET A | 553 | −1.514 | 17.756 | 78.686 | 1.00 | 31.97 | C |

| | | | | | | | | | | | |
|---|---|---|---|---|---|---|---|---|---|---|---|
| ANISOU | 1257 | CE | MET A | 553 | 3853 | 4196 | 4098 | −122 | 147 | 142 | C |

| | | | | | | | | | | |
|---|---|---|---|---|---|---|---|---|---|---|
| ATOM | 1258 | N | GLU A | 554 | 0.511 | 15.163 | 75.164 | 1.00 | 25.97 | N |

| | | | | | | | | | | | |
|---|---|---|---|---|---|---|---|---|---|---|---|
| ANISOU | 1258 | N | GLU A | 554 | 2964 | 3201 | 3703 | −86 | 4 | 102 | N |

| | | | | | | | | | | |
|---|---|---|---|---|---|---|---|---|---|---|
| ATOM | 1259 | CA | GLU A | 554 | 0.181 | 15.005 | 73.757 | 1.00 | 27.16 | C |

| | | | | | | | | | | | |
|---|---|---|---|---|---|---|---|---|---|---|---|
| ANISOU | 1259 | CA | GLU A | 554 | 3313 | 3308 | 3698 | −127 | −18 | 1 | C |

| | | | | | | | | | | |
|---|---|---|---|---|---|---|---|---|---|---|
| ATOM | 1260 | C | GLU A | 554 | 1.098 | 14.065 | 73.003 | 1.00 | 26.08 | C |

| | | | | | | | | | | | |
|---|---|---|---|---|---|---|---|---|---|---|---|
| ANISOU | 1260 | C | GLU A | 554 | 3038 | 3297 | 3576 | −196 | −102 | 47 | C |

| | | | | | | | | | | |
|---|---|---|---|---|---|---|---|---|---|---|
| ATOM | 1261 | O | GLU A | 554 | 0.659 | 13.172 | 72.252 | 1.00 | 26.70 | O |

| | | | | | | | | | | | |
|---|---|---|---|---|---|---|---|---|---|---|---|
| ANISOU | 1261 | O | GLU A | 554 | 3074 | 3283 | 3790 | −361 | −293 | 12 | O |

| | | | | | | | | | | |
|---|---|---|---|---|---|---|---|---|---|---|
| ATOM | 1262 | CB | GLU A | 554 | 0.117 | 16.374 | 73.050 | 1.00 | 29.34 | C |

| | | | | | | | | | | | |
|---|---|---|---|---|---|---|---|---|---|---|---|
| ANISOU | 1262 | CB | GLU A | 554 | 3799 | 3529 | 3820 | −210 | −66 | 159 | C |

| | | | | | | | | | | |
|---|---|---|---|---|---|---|---|---|---|---|
| ATOM | 1263 | CG | GLU A | 554 | 0.898 | 17.308 | 73.689 | 1.00 | 32.77 | C |

| | | | | | | | | | | | |
|---|---|---|---|---|---|---|---|---|---|---|---|
| ANISOU | 1263 | CG | GLU A | 554 | 4107 | 4049 | 4294 | 11 | 27 | −15 | C |

| | | | | | | | | | | |
|---|---|---|---|---|---|---|---|---|---|---|
| ATOM | 1264 | CD | GLU A | 554 | −2.343 | 16.855 | 73.550 | 1.00 | 35.94 | C |

| | | | | | | | | | | | |
|---|---|---|---|---|---|---|---|---|---|---|---|
| ANISOU | 1264 | CD | GLU A | 554 | 4309 | 4585 | 4762 | −137 | −26 | 5 | C |

| | | | | | | | | | | |
|---|---|---|---|---|---|---|---|---|---|---|
| ATOM | 1265 | OE1 | GLU A | 554 | −2.631 | 16.312 | 72.470 | 1.00 | 37.57 | O |

| | | | | | | | | | | | |
|---|---|---|---|---|---|---|---|---|---|---|---|
| ANISOU | 1265 | OE1 | GLU A | 554 | 4520 | 4980 | 4775 | −228 | −112 | −40 | O |

| | | | | | | | | | | |
|---|---|---|---|---|---|---|---|---|---|---|
| ATOM | 1266 | OE2 | GLU A | 554 | −3.104 | 17.042 | 74.519 | 1.00 | 37.27 | O |

| | | | | | | | | | | | |
|---|---|---|---|---|---|---|---|---|---|---|---|
| ANISOU | 1266 | OE2 | GLU A | 554 | 4434 | 4746 | 4980 | −161 | 93 | 15 | O |

| | | | | | | | | | | |
|---|---|---|---|---|---|---|---|---|---|---|
| ATOM | 1267 | N | GLU A | 555 | 2.411 | 14.166 | 73.183 | 1.00 | 24.96 | N |

| | | | | | | | | | | | |
|---|---|---|---|---|---|---|---|---|---|---|---|
| ANISOU | 1267 | N | GLU A | 555 | 2975 | 3048 | 3461 | −271 | −172 | 78 | N |

| | | | | | | | | | | |
|---|---|---|---|---|---|---|---|---|---|---|
| ATOM | 1268 | CA | GLU A | 555 | 3.378 | 13.327 | 72.502 | 1.00 | 24.95 | C |

| | | | | | | | | | | | |
|---|---|---|---|---|---|---|---|---|---|---|---|
| ANISOU | 1268 | CA | GLU A | 555 | 2759 | 3221 | 3463 | −315 | −182 | −14 | C |

| | | | | | | | | | | |
|---|---|---|---|---|---|---|---|---|---|---|
| ATOM | 1269 | C | GLU A | 555 | 3.218 | 11.868 | 72.897 | 1.00 | 26.77 | C |

| | | | | | | | | | | | |
|---|---|---|---|---|---|---|---|---|---|---|---|
| ANISOU | 1269 | C | GLU A | 555 | 3176 | 3310 | 3686 | −84 | −143 | 59 | C |

| | | | | | | | | | | |
|---|---|---|---|---|---|---|---|---|---|---|
| ATOM | 1270 | O | GLU A | 555 | 3.437 | 10.979 | 72.081 | 1.00 | 29.06 | O |

| | | | | | | | | | | | |
|---|---|---|---|---|---|---|---|---|---|---|---|
| ANISOU | 1270 | O | GLU A | 555 | 3680 | 3496 | 3868 | −228 | −53 | −99 | O |

| | | | | | | | | | | |
|---|---|---|---|---|---|---|---|---|---|---|
| ATOM | 1271 | CB | GLU A | 555 | 4.818 | 13.829 | 72.834 | 1.00 | 25.12 | C |

| | | | | | | | | | | | |
|---|---|---|---|---|---|---|---|---|---|---|---|
| ANISOU | 1271 | CB | GLU A | 555 | 2780 | 3207 | 3557 | −214 | −142 | −116 | C |

| | | | | | | | | | | |
|---|---|---|---|---|---|---|---|---|---|---|
| ATOM | 1272 | CG | GLU A | 555 | 5.884 | 13.047 | 72.095 | 1.00 | 24.32 | C |

| | | | | | | | | | | | |
|---|---|---|---|---|---|---|---|---|---|---|---|
| ANISOU | 1272 | CG | GLU A | 555 | 2804 | 3053 | 3384 | −277 | −116 | −104 | C |

| | | | | | | | | | | |
|---|---|---|---|---|---|---|---|---|---|---|
| ATOM | 1273 | CD | GLU A | 555 | 5.802 | 13.180 | 70.592 | 1.00 | 25.45 | C |

| | | | | | | | | | | | |
|---|---|---|---|---|---|---|---|---|---|---|---|
| ANISOU | 1273 | CD | GLU A | 555 | 3006 | 3215 | 3449 | −194 | −128 | −23 | C |

| | | | | | | | | | | |
|---|---|---|---|---|---|---|---|---|---|---|
| ATOM | 1274 | OE1 | GLU A | 555 | 5.283 | 14.191 | 70.069 | 1.00 | 26.52 | O |

| | | | | | | | | | | | |
|---|---|---|---|---|---|---|---|---|---|---|---|
| ANISOU | 1274 | OE1 | GLU A | 555 | 3194 | 3175 | 3709 | −237 | −137 | −15 | O |

| | | | | | | | | | | |
|---|---|---|---|---|---|---|---|---|---|---|
| ATOM | 1275 | OE2 | GLU A | 555 | 6.302 | 12.257 | 69.917 | 1.00 | 25.01 | O |

| | | | | | | | | | | | |
|---|---|---|---|---|---|---|---|---|---|---|---|
| ANISOU | 1275 | OE2 | GLU A | 555 | 2964 | 3115 | 3424 | −181 | −95 | 68 | O |

| | | | | | | | | | | |
|---|---|---|---|---|---|---|---|---|---|---|
| ATOM | 1276 | N | SER A | 556 | 2.807 | 11.611 | 74.138 | 1.00 | 27.38 | N |

| | | | | | | | | | | | |
|---|---|---|---|---|---|---|---|---|---|---|---|
| ANISOU | 1276 | N | SER A | 556 | 3426 | 3272 | 3706 | −160 | −58 | 69 | N |

| | | | | | | | | | | |
|---|---|---|---|---|---|---|---|---|---|---|
| ATOM | 1277 | CA | SER A | 556 | 2.605 | 10.236 | 74.590 | 1.00 | 28.32 | C |

| | | | | | | | | | | | |
|---|---|---|---|---|---|---|---|---|---|---|---|
| ANISOU | 1277 | CA | SER A | 556 | 3570 | 3307 | 3884 | −260 | −158 | 76 | C |

| | | | | | | | | | | |
|---|---|---|---|---|---|---|---|---|---|---|
| ATOM | 1278 | C | SER A | 556 | 1.213 | 9.710 | 74.262 | 1.00 | 29.09 | C |

| | | | | | | | | | | | |
|---|---|---|---|---|---|---|---|---|---|---|---|
| ANISOU | 1278 | C | SER A | 556 | 3485 | 3512 | 4057 | −240 | −38 | 34 | C |

| | | | | | | | | | | |
|---|---|---|---|---|---|---|---|---|---|---|
| ATOM | 1279 | O | SER A | 556 | 0.870 | 8.642 | 74.794 | 1.00 | 30.33 | O |

| | | | | | | | | | | | |
|---|---|---|---|---|---|---|---|---|---|---|---|
| ANISOU | 1279 | O | SER A | 556 | 3797 | 3389 | 4339 | −335 | −138 | 2 | O |

| | | | | | | | | | | |
|---|---|---|---|---|---|---|---|---|---|---|
| ATOM | 1280 | CB | SER A | 556 | 2.796 | 10.137 | 76.107 | 1.00 | 27.22 | C |

| | | | | | | | | | | | |
|---|---|---|---|---|---|---|---|---|---|---|---|
| ANISOU | 1280 | CB | SER A | 556 | 3373 | 3170 | 3800 | −195 | −28 | 63 | C |

| | | | | | | | | | | |
|---|---|---|---|---|---|---|---|---|---|---|
| ATOM | 1281 | OG | SER A | 556 | 1.787 | 10.829 | 76.803 | 1.00 | 26.02 | O |

| | | | | | | | | | | | |
|---|---|---|---|---|---|---|---|---|---|---|---|
| ANISOU | 1281 | OG | SER A | 556 | 2773 | 3122 | 3989 | −399 | −131 | 121 | O |

| | | | | | | | | | | |
|---|---|---|---|---|---|---|---|---|---|---|
| ATOM | 1282 | N | LYS A | 557 | 0.404 | 10.484 | 73.579 | 1.00 | 30.03 | N |

| | | | | | | | | | | | |
|---|---|---|---|---|---|---|---|---|---|---|---|
| ANISOU | 1282 | N | LYS A | 557 | 3768 | 3536 | 4104 | −271 | −156 | 16 | N |

| | | | | | | | | | | |
|---|---|---|---|---|---|---|---|---|---|---|
| ATOM | 1283 | CA | LYS A | 557 | −0.977 | 10.070 | 73.286 | 1.00 | 32.80 | C |

| | | | | | | | | | | | |
|---|---|---|---|---|---|---|---|---|---|---|---|
| ANISOU | 1283 | CA | LYS A | 557 | 3756 | 4228 | 4479 | −220 | −63 | 41 | C |

| | | | | | | | | | | |
|---|---|---|---|---|---|---|---|---|---|---|
| ATOM | 1284 | C | LYS A | 557 | −1.721 | 9.825 | 74.577 | 1.00 | 33.23 | C |

| | | | | | | | | | | | |
|---|---|---|---|---|---|---|---|---|---|---|---|
| ANISOU | 1284 | C | LYS A | 557 | 3980 | 4202 | 4442 | −136 | −55 | 37 | C |

| | | | | | | | | | | |
|---|---|---|---|---|---|---|---|---|---|---|
| ATOM | 1285 | O | LYS A | 557 | −2.440 | 8.813 | 74.727 | 1.00 | 34.82 | O |

| | | | | | | | | | | | |
|---|---|---|---|---|---|---|---|---|---|---|---|
| ANISOU | 1285 | O | LYS A | 557 | 4210 | 4271 | 4749 | −324 | −139 | −29 | O |

| | | | | | | | | | | |
|---|---|---|---|---|---|---|---|---|---|---|
| ATOM | 1296 | CB | LYS A | 557 | −0.924 | 8.833 | 72.392 | 1.00 | 34.77 | C |

| | | | | | | | | | | | |
|---|---|---|---|---|---|---|---|---|---|---|---|
| ANISOU | 1286 | CB | LYS A | 557 | 4195 | 4368 | 4648 | −149 | −102 | −73 | C |

| | | | | | | | | | | |
|---|---|---|---|---|---|---|---|---|---|---|
| ATOM | 1287 | CG | LYS A | 557 | −0.517 | 9.177 | 70.965 | 1.00 | 38.01 | C |

| | | | | | | | | | | | |
|---|---|---|---|---|---|---|---|---|---|---|---|
| ANISOU | 1287 | CG | LYS A | 557 | 4704 | 4951 | 4787 | −114 | −3 | 33 | C |

| | | | | | | | | | | |
|---|---|---|---|---|---|---|---|---|---|---|
| ATOM | 1288 | CD | LYS A | 557 | −0.088 | 7.923 | 70.218 | 1.00 | 40.41 | C |

| | | | | | | | | | | | |
|---|---|---|---|---|---|---|---|---|---|---|---|
| ANISOU | 1288 | CD | LYS A | 557 | 5065 | 5095 | 5196 | −3 | −16 | −90 | C |

| | | | | | | | | | | |
|---|---|---|---|---|---|---|---|---|---|---|
| ATOM | 1289 | CE | LYS A | 557 | 0.416 | 8.299 | 68.825 | 1.00 | 42.43 | C |

| | | | | | | | | | | | |
|---|---|---|---|---|---|---|---|---|---|---|---|
| ANISOU | 1289 | CE | LYS A | 557 | 5443 | 5425 | 5252 | −33 | 16 | 9 | C |

| | | | | | | | | | | |
|---|---|---|---|---|---|---|---|---|---|---|
| ATOM | 1290 | NZ | LYS A | 557 | 1.635 | 7.512 | 68.466 | 1.00 | 44.10 | N |

| | | | | | | | | | | | |
|---|---|---|---|---|---|---|---|---|---|---|---|
| ANISOU | 1290 | NZ | LYS A | 557 | 5512 | 5671 | 5574 | 47 | 47 | −22 | N |

| | | | | | | | | | | |
|---|---|---|---|---|---|---|---|---|---|---|
| ATOM | 1291 | N | HIS A | 558 | −1.604 | 10.765 | 75.502 | 1.00 | 32.60 | N |

| | | | | | | | | | | | |
|---|---|---|---|---|---|---|---|---|---|---|---|
| ANISOU | 1291 | N | HIS A | 558 | 3743 | 4183 | 4463 | −187 | −75 | 16 | N |

| | | | | | | | | | | |
|---|---|---|---|---|---|---|---|---|---|---|
| ATOM | 1292 | CA | HIS A | 558 | −2.246 | 10.757 | 76.800 | 1.00 | 33.68 | C |

| | | | | | | | | | | | |
|---|---|---|---|---|---|---|---|---|---|---|---|
| ANISOU | 1292 | CA | HIS A | 558 | 4072 | 4242 | 4482 | −99 | −14 | 35 | C |

| | | | | | | | | | | |
|---|---|---|---|---|---|---|---|---|---|---|
| ATOM | 1293 | C | HIS A | 558 | −1.999 | 9.509 | 77.618 | 1.00 | 33.69 | C |

| | | | | | | | | | | | |
|---|---|---|---|---|---|---|---|---|---|---|---|
| ANISOU | 1293 | C | HIS A | 558 | 4004 | 4189 | 4607 | −55 | 5 | 17 | C |

| | | | | | | | | | | |
|---|---|---|---|---|---|---|---|---|---|---|
| ATOM | 1294 | O | HIS A | 558 | −2.929 | 9.008 | 78.287 | 1.00 | 35.32 | O |

| | | | | | | | | | | | |
|---|---|---|---|---|---|---|---|---|---|---|---|
| ANISOU | 1294 | O | HIS A | 558 | 4271 | 4416 | 4731 | −80 | 201 | 169 | O |

| | | | | | | | | | | |
|---|---|---|---|---|---|---|---|---|---|---|
| ATOM | 1295 | CB | HIS A | 558 | −3.767 | 11.022 | 76.623 | 1.00 | 35.06 | C |

| | | | | | | | | | | | |
|---|---|---|---|---|---|---|---|---|---|---|---|
| ANISOU | 1295 | CB | HIS A | 558 | 4096 | 4524 | 4702 | −69 | −45 | 36 | C |

| | | | | | | | | | | |
|---|---|---|---|---|---|---|---|---|---|---|
| ATOM | 1296 | CG | HIS A | 558 | −3.906 | 12.416 | 76.059 | 1.00 | 36.32 | C |

| | | | | | | | | | | | |
|---|---|---|---|---|---|---|---|---|---|---|---|
| ANISOU | 1296 | CG | HIS A | 558 | 4365 | 4626 | 4807 | 30 | −17 | 71 | C |

| | | | | | | | | | | |
|---|---|---|---|---|---|---|---|---|---|---|
| ATOM | 1297 | ND1 | HIS A | 558 | −4.002 | 12.646 | 74.705 | 1.00 | 37.84 | N |

| | | | | | | | | | | | |
|---|---|---|---|---|---|---|---|---|---|---|---|
| ANISOU | 1297 | ND1 | HIS A | 558 | 4643 | 4878 | 4855 | 33 | −26 | 46 | N |

| | | | | | | | | | | |
|---|---|---|---|---|---|---|---|---|---|---|
| ATOM | 1298 | CD2 | HIS A | 558 | −3.882 | 13.615 | 76.676 | 1.00 | 36.40 | C |

| | | | | | | | | | | | |
|---|---|---|---|---|---|---|---|---|---|---|---|
| ANISOU | 1298 | CD2 | HIS A | 558 | 4386 | 4659 | 4786 | −21 | −48 | 6 | C |

| | | | | | | | | | | |
|---|---|---|---|---|---|---|---|---|---|---|
| ATOM | 1299 | CE1 | HIS A | 558 | −4.071 | 13.957 | 74.501 | 1.00 | 37.74 | C |

| | | | | | | | | | | | |
|---|---|---|---|---|---|---|---|---|---|---|---|
| ANISOU | 1299 | CE1 | HIS A | 558 | 4664 | 4836 | 4839 | −51 | 26 | 9 | C |

| | | | | | | | | | | |
|---|---|---|---|---|---|---|---|---|---|---|
| ATOM | 1300 | NE2 | HIS A | 558 | −4.001 | 14.557 | 75.675 | 1.00 | 37.69 | N |

| | | | | | | | | | | | |
|---|---|---|---|---|---|---|---|---|---|---|---|
| ANISOU | 1300 | NE2 | HIS A | 558 | 4664 | 4849 | 4806 | −83 | 17 | 46 | N |

| | | | | | | | | | | |
|---|---|---|---|---|---|---|---|---|---|---|
| ATOM | 1301 | N | THR A | 559 | −0.775 | 8.998 | 77.602 | 1.00 | 31.31 | N |

| | | | | | | | | | | | |
|---|---|---|---|---|---|---|---|---|---|---|---|
| ANISOU | 1301 | N | THR A | 559 | 3768 | 3728 | 4399 | −297 | −91 | 22 | N |

| | | | | | | | | | | |
|---|---|---|---|---|---|---|---|---|---|---|
| ATOM | 1302 | CA | THR A | 559 | −0.446 | 7.850 | 78.422 | 1.00 | 31.64 | C |

| | | | | | | | | | | | |
|---|---|---|---|---|---|---|---|---|---|---|---|
| ANISOU | 1302 | CA | THR A | 559 | 3894 | 3908 | 4220 | −309 | −12 | 118 | C |

| | | | | | | | | | | |
|---|---|---|---|---|---|---|---|---|---|---|
| ATOM | 1303 | C | THR A | 559 | 0.316 | 8.269 | 79.664 | 1.00 | 31.07 | C |

| | | | | | | | | | | | |
|---|---|---|---|---|---|---|---|---|---|---|---|
| ANISOU | 1303 | C | THR A | 559 | 3985 | 3721 | 4100 | −247 | 39 | 160 | C |

| | | | | | | | | | | |
|---|---|---|---|---|---|---|---|---|---|---|
| ATOM | 1304 | O | THR A | 559 | 0.378 | 7.551 | 80.671 | 1.00 | 31.04 | O |

| | | | | | | | | | | | |
|---|---|---|---|---|---|---|---|---|---|---|---|
| ANISOU | 1304 | O | THR A | 559 | 4015 | 3561 | 4218 | −350 | 46 | 222 | O |

| | | | | | | | | | | |
|---|---|---|---|---|---|---|---|---|---|---|
| ATOM | 1305 | CB | THR A | 559 | 0.341 | 6.761 | 77.702 | 1.00 | 32.73 | C |

| | | | | | | | | | | | |
|---|---|---|---|---|---|---|---|---|---|---|---|
| ANISOU | 1305 | CB | THR A | 559 | 4065 | 4057 | 4314 | −146 | −24 | 73 | C |

| | | | | | | | | | | |
|---|---|---|---|---|---|---|---|---|---|---|
| ATOM | 1306 | OG1 | THR A | 559 | 1.490 | 7.238 | 76.996 | 1.00 | 32.65 | O |

| | | | | | | | | | | | |
|---|---|---|---|---|---|---|---|---|---|---|---|
| ANISOU | 1306 | OG1 | THR A | 559 | 4065 | 3911 | 4432 | −285 | −45 | 66 | O |

| | | | | | | | | | | |
|---|---|---|---|---|---|---|---|---|---|---|
| ATOM | 1307 | CG2 | THR A | 559 | −0.505 | 6.013 | 76.659 | 1.00 | 32.81 | C |

| | | | | | | | | | | | |
|---|---|---|---|---|---|---|---|---|---|---|---|
| ANISOU | 1307 | CG2 | THR A | 559 | 4071 | 4159 | 4235 | −215 | −79 | 135 | C |

| | | | | | | | | | | |
|---|---|---|---|---|---|---|---|---|---|---|
| ATOM | 1308 | N | ILE A | 560 | 1.098 | 9.370 | 79.523 | 1.00 | 28.80 | N |

| | | | | | | | | | | | |
|---|---|---|---|---|---|---|---|---|---|---|---|
| ANISOU | 1308 | N | ILE A | 560 | 3655 | 3444 | 3845 | −136 | −90 | 99 | N |

| | | | | | | | | | | |
|---|---|---|---|---|---|---|---|---|---|---|
| ATOM | 1309 | CA | ILE A | 560 | 1.937 | 9.804 | 80.635 | 1.00 | 28.76 | C |

| | | | | | | | | | | | |
|---|---|---|---|---|---|---|---|---|---|---|---|
| ANISOU | 1309 | CA | ILE A | 560 | 3735 | 3393 | 3797 | −4 | −42 | 47 | C |

| | | | | | | | | | | |
|---|---|---|---|---|---|---|---|---|---|---|
| ATOM | 1310 | C | ILE A | 560 | 1.943 | 11.325 | 80.725 | 1.00 | 27.06 | C |

| | | | | | | | | | | | |
|---|---|---|---|---|---|---|---|---|---|---|---|
| ANISOU | 1310 | C | ILE A | 560 | 3319 | 3330 | 3632 | −181 | −12 | 97 | C |

| | | | | | | | | | | |
|---|---|---|---|---|---|---|---|---|---|---|
| ATOM | 1311 | O | ILE A | 560 | 1.502 | 12.002 | 79.780 | 1.00 | 25.47 | O |

| | | | | | | | | | | | |
|---|---|---|---|---|---|---|---|---|---|---|---|
| ANISOU | 1311 | O | ILE A | 560 | 3138 | 3136 | 3404 | −365 | −109 | −50 | O |

| | | | | | | | | | | |
|---|---|---|---|---|---|---|---|---|---|---|
| ATOM | 1312 | CB | ILE A | 560 | 3.402 | 9.351 | 80.550 | 1.00 | 30.47 | C |

| | | | | | | | | | | | |
|---|---|---|---|---|---|---|---|---|---|---|---|
| ANISOU | 1312 | CB | ILE A | 560 | 3790 | 3772 | 4013 | 16 | 16 | 50 | C |

| | | | | | | | | | | |
|---|---|---|---|---|---|---|---|---|---|---|
| ATOM | 1313 | CG1 | ILE A | 560 | 4.066 | 9.737 | 79.235 | 1.00 | 30.69 | C |

| | | | | | | | | | | | |
|---|---|---|---|---|---|---|---|---|---|---|---|
| ANISOU | 1313 | CG1 | ILE A | 560 | 3822 | 3865 | 3973 | 21 | 15 | 40 | C |

| | | | | | | | | | | |
|---|---|---|---|---|---|---|---|---|---|---|
| ATOM | 1314 | CG2 | ILE A | 560 | 3.567 | 7.868 | 80.869 | 1.00 | 30.96 | C |

| | | | | | | | | | | | |
|---|---|---|---|---|---|---|---|---|---|---|---|
| ANISOU | 1314 | CG2 | ILE A | 560 | 3886 | 3731 | 4145 | −19 | −16 | −20 | C |

| | | | | | | | | | | |
|---|---|---|---|---|---|---|---|---|---|---|
| ATOM | 1315 | CD1 | ILE A | 560 | 5.570 | 9.465 | 79.241 | 1.00 | 30.76 | C |

| | | | | | | | | | | | |
|---|---|---|---|---|---|---|---|---|---|---|---|
| ANISOU | 1315 | CD1 | ILE A | 560 | 3679 | 3999 | 4009 | −81 | −71 | 48 | C |

| | | | | | | | | | | |
|---|---|---|---|---|---|---|---|---|---|---|
| ATOM | 1316 | N | GLY A | 561 | 2.295 | 11.821 | 81.907 | 1.00 | 26.90 | N |

| | | | | | | | | | | | |
|---|---|---|---|---|---|---|---|---|---|---|---|
| ANISOU | 1316 | N | GLY A | 561 | 3362 | 3190 | 3669 | −126 | −48 | 83 | N |

| | | | | | | | | | | |
|---|---|---|---|---|---|---|---|---|---|---|
| ATOM | 1317 | CA | GLY A | 561 | 2.398 | 13.268 | 82.091 | 1.00 | 26.89 | C |

| | | | | | | | | | | | |
|---|---|---|---|---|---|---|---|---|---|---|---|
| ANISOU | 1317 | CA | GLY A | 561 | 3295 | 3170 | 3751 | −242 | −57 | 141 | C |

| | | | | | | | | | | |
|---|---|---|---|---|---|---|---|---|---|---|
| ATOM | 1318 | C | GLY A | 561 | 1.403 | 13.967 | 82.959 | 1.00 | 29.30 | C |

| | | | | | | | | | | | |
|---|---|---|---|---|---|---|---|---|---|---|---|
| ANISOU | 1318 | C | GLY A | 561 | 3630 | 3540 | 3963 | −83 | 22 | 29 | C |

| | | | | | | | | | | |
|---|---|---|---|---|---|---|---|---|---|---|
| ATOM | 1319 | O | GLY A | 561 | 1.754 | 14.916 | 83.667 | 1.00 | 28.44 | O |

| | | | | | | | | | | | |
|---|---|---|---|---|---|---|---|---|---|---|---|
| ANISOU | 1319 | O | GLY A | 561 | 3166 | 3646 | 3994 | −347 | 60 | 87 | O |

| | | | | | | | | | | |
|---|---|---|---|---|---|---|---|---|---|---|
| ATOM | 1320 | N | GLU A | 562 | 0.123 | 13.551 | 83.033 | 1.00 | 31.92 | N |

| | | | | | | | | | | | |
|---|---|---|---|---|---|---|---|---|---|---|---|
| ANISOU | 1320 | N | GLU A | 562 | 3812 | 3912 | 4403 | −289 | 12 | 88 | N |

| | | | | | | | | | | |
|---|---|---|---|---|---|---|---|---|---|---|
| ATOM | 1321 | CA | GLU A | 562 | −0.894 | 14.265 | 83.803 | 1.00 | 33.43 | C |

| | | | | | | | | | | | |
|---|---|---|---|---|---|---|---|---|---|---|---|
| ANISOU | 1321 | CA | GLU A | 562 | 4179 | 4112 | 4413 | −85 | 83 | 39 | C |

| | | | | | | | | | | |
|---|---|---|---|---|---|---|---|---|---|---|
| ATOM | 1322 | C | GLU A | 562 | −0.685 | 14.344 | 85.300 | 1.00 | 31.31 | C |

| | | | | | | | | | | | |
|---|---|---|---|---|---|---|---|---|---|---|---|
| ANISOU | 1322 | C | GLU A | 562 | 3697 | 3829 | 4369 | −172 | 73 | 120 | C |

| | | | | | | | | | | |
|---|---|---|---|---|---|---|---|---|---|---|
| ATOM | 1323 | O | GLU A | 562 | −1.151 | 15.281 | 85.988 | 1.00 | 30.72 | O |

| | | | | | | | | | | | |
|---|---|---|---|---|---|---|---|---|---|---|---|
| ANISOU | 1323 | O | GLU A | 562 | 3742 | 3700 | 4229 | −265 | 131 | 229 | O |

| | | | | | | | | | | |
|---|---|---|---|---|---|---|---|---|---|---|
| ATOM | 1324 | CB | GLU A | 562 | −2.281 | 13.699 | 83.422 | 1.00 | 38.48 | C |

| | | | | | | | | | | | |
|---|---|---|---|---|---|---|---|---|---|---|---|
| ANISOU | 1324 | CB | GLU A | 562 | 4420 | 5013 | 5189 | −158 | −73 | −61 | C |

| | | | | | | | | | | |
|---|---|---|---|---|---|---|---|---|---|---|
| ATOM | 1325 | CG | GLU A | 562 | −2.677 | 12.442 | 84.171 | 1.00 | 43.17 | C |

| | | | | | | | | | | | |
|---|---|---|---|---|---|---|---|---|---|---|---|
| ANISOU | 1325 | CG | GLU A | 562 | 5524 | 5245 | 5634 | −35 | 53 | 163 | C |

| | | | | | | | | | | |
|---|---|---|---|---|---|---|---|---|---|---|
| ATOM | 1326 | CD | GLU A | 562 | −4.082 | 11.974 | 83.808 | 1.00 | 46.65 | C |

| | | | | | | | | | | | |
|---|---|---|---|---|---|---|---|---|---|---|---|
| ANISOU | 1326 | CD | GLU A | 562 | 5690 | 5941 | 6094 | −89 | −65 | 13 | C |

| | | | | | | | | | | |
|---|---|---|---|---|---|---|---|---|---|---|
| ATOM | 1327 | OE1 | GLU A | 562 | −4.977 | 12.830 | 83.576 | 1.00 | 47.41 | O |

| | | | | | | | | | | | |
|---|---|---|---|---|---|---|---|---|---|---|---|
| ANISOU | 1327 | OE1 | GLU A | 562 | 5810 | 5960 | 6244 | 15 | −30 | −13 | O |

| | | | | | | | | | | |
|---|---|---|---|---|---|---|---|---|---|---|
| ATOM | 1328 | OE2 | GLU A | 562 | −4.284 | 10.735 | 83.751 | 1.00 | 49.19 | O |

| | | | | | | | | | | | |
|---|---|---|---|---|---|---|---|---|---|---|---|
| ANISOU | 1328 | OE2 | GLU A | 562 | 6253 | 6001 | 6437 | −48 | −10 | 42 | O |

| | | | | | | | | | | |
|---|---|---|---|---|---|---|---|---|---|---|
| ATOM | 1329 | N | ASP A | 563 | 0.084 | 13.431 | 85.890 | 1.00 | 28.74 | N |

| | | | | | | | | | | | |
|---|---|---|---|---|---|---|---|---|---|---|---|
| ANISOU | 1329 | N | ASP A | 563 | 3271 | 3553 | 4096 | −421 | 112 | 69 | N |

| | | | | | | | | | | |
|---|---|---|---|---|---|---|---|---|---|---|
| ATOM | 1330 | CA | ASP A | 563 | 0.382 | 13.368 | 87.291 | 1.00 | 29.52 | C |

| | | | | | | | | | | | |
|---|---|---|---|---|---|---|---|---|---|---|---|
| ANISOU | 1330 | CA | ASP A | 563 | 3462 | 3712 | 4041 | −212 | 185 | 74 | C |

| | | | | | | | | | | |
|---|---|---|---|---|---|---|---|---|---|---|
| ATOM | 1331 | C | ASP A | 563 | 1.820 | 13.792 | 87.605 | 1.00 | 28.85 | C |

| | | | | | | | | | | | |
|---|---|---|---|---|---|---|---|---|---|---|---|
| ANISOU | 1331 | C | ASP A | 563 | 3391 | 3756 | 3815 | −92 | 68 | 66 | C |

| | | | | | | | | | | |
|---|---|---|---|---|---|---|---|---|---|---|
| ATOM | 1332 | O | ASP A | 563 | 2.248 | 13.622 | 88.744 | 1.00 | 28.02 | O |

| | | | | | | | | | | | |
|---|---|---|---|---|---|---|---|---|---|---|---|
| ANISOU | 1332 | O | ASP A | 563 | 3147 | 3722 | 3775 | −141 | 263 | 121 | O |

| | | | | | | | | | | |
|---|---|---|---|---|---|---|---|---|---|---|
| ATOM | 1333 | CB | ASP A | 563 | 0.161 | 11.936 | 87.827 | 1.00 | 32.05 | C |

| | | | | | | | | | | | |
|---|---|---|---|---|---|---|---|---|---|---|---|
| ANISOU | 1333 | CB | ASP A | 563 | 4080 | 3863 | 4233 | −219 | 158 | 177 | C |

| | | | | | | | | | | |
|---|---|---|---|---|---|---|---|---|---|---|
| ATOM | 1334 | CG | ASP A | 563 | 1.014 | 10.881 | 87.188 | 1.00 | 33.86 | C |

| | | | | | | | | | | | |
|---|---|---|---|---|---|---|---|---|---|---|---|
| ANISOU | 1334 | CG | ASP A | 563 | 4361 | 4082 | 4421 | −81 | 192 | 79 | C |

| | | | | | | | | | | |
|---|---|---|---|---|---|---|---|---|---|---|
| ATOM | 1335 | OD1 | ASP A | 563 | 1.761 | 11.179 | 86.221 | 1.00 | 33.79 | O |

| | | | | | | | | | | | |
|---|---|---|---|---|---|---|---|---|---|---|---|
| ANISOU | 1335 | OD1 | ASP A | 563 | 4295 | 3981 | 4562 | −259 | 185 | 232 | O |

| | | | | | | | | | | |
|---|---|---|---|---|---|---|---|---|---|---|
| ATOM | 1336 | OD2 | ASP A | 563 | 0.998 | 9.670 | 87.573 | 1.00 | 35.37 | O |

| | | | | | | | | | | | |
|---|---|---|---|---|---|---|---|---|---|---|---|
| ANISOU | 1336 | OD2 | ASP A | 563 | 4698 | 4219 | 4524 | −95 | 223 | 297 | O |

| | | | | | | | | | | |
|---|---|---|---|---|---|---|---|---|---|---|
| ATOM | 1337 | N | PHE A | 564 | 2.504 | 14.388 | 86.608 | 1.00 | 28.32 | N |

| | | | | | | | | | | | |
|---|---|---|---|---|---|---|---|---|---|---|---|
| ANISOU | 1337 | N | PHE A | 564 | 3435 | 3509 | 3816 | −134 | 30 | 16 | N |

| | | | | | | | | | | |
|---|---|---|---|---|---|---|---|---|---|---|
| ATOM | 1338 | CA | PHE A | 564 | 3.869 | 14.845 | 86.925 | 1.00 | 26.63 | C |

| | | | | | | | | | | | |
|---|---|---|---|---|---|---|---|---|---|---|---|
| ANISOU | 1338 | CA | PHE A | 564 | 3161 | 3432 | 3526 | −54 | 242 | 103 | C |

| | | | | | | | | | | |
|---|---|---|---|---|---|---|---|---|---|---|
| ATOM | 1339 | C | PHE A | 564 | 3.870 | 16.034 | 87.880 | 1.00 | 27.46 | C |

| | | | | | | | | | | | |
|---|---|---|---|---|---|---|---|---|---|---|---|
| ANISOU | 1339 | C | PHE A | 564 | 3417 | 3352 | 3666 | −121 | 181 | 107 | C |

| | | | | | | | | | | |
|---|---|---|---|---|---|---|---|---|---|---|
| ATOM | 1340 | O | PHE A | 564 | 2.951 | 16.876 | 87.830 | 1.00 | 28.83 | O |

| | | | | | | | | | | | |
|---|---|---|---|---|---|---|---|---|---|---|---|
| ANISOU | 1340 | O | PHE A | 564 | 3359 | 3570 | 4023 | 21 | 67 | 168 | O |

| | | | | | | | | | | |
|---|---|---|---|---|---|---|---|---|---|---|
| ATOM | 1341 | CB | PHE A | 564 | 4.619 | 15.226 | 85.635 | 1.00 | 25.30 | C |

| | | | | | | | | | | | |
|---|---|---|---|---|---|---|---|---|---|---|---|
| ANISOU | 1341 | CB | PHE A | 564 | 3155 | 3120 | 3337 | −60 | 166 | 16 | C |

| | | | | | | | | | | |
|---|---|---|---|---|---|---|---|---|---|---|
| ATOM | 1342 | CG | PHE A | 564 | 5.184 | 14.097 | 84.823 | 1.00 | 23.86 | C |

| | | | | | | | | | | | |
|---|---|---|---|---|---|---|---|---|---|---|---|
| ANISOU | 1342 | CG | PHE A | 564 | 2835 | 2962 | 3269 | −76 | 81 | 31 | C |

| | | | | | | | | | | |
|---|---|---|---|---|---|---|---|---|---|---|
| ATOM | 1343 | CD1 | PHE A | 564 | 4.664 | 12.810 | 84.786 | 1.00 | 25.17 | C |

| | | | | | | | | | | | |
|---|---|---|---|---|---|---|---|---|---|---|---|
| ANISOU | 1343 | CD1 | PHE A | 564 | 3212 | 2965 | 3386 | −98 | 89 | 83 | C |

| | | | | | | | | | | |
|---|---|---|---|---|---|---|---|---|---|---|
| ATOM | 1344 | CD2 | PHE A | 564 | 6.279 | 14.362 | 83.997 | 1.00 | 24.60 | C |

| | | | | | | | | | | | |
|---|---|---|---|---|---|---|---|---|---|---|---|
| ANISOU | 1344 | CD2 | PHE A | 564 | 3173 | 3039 | 3135 | −100 | 89 | 206 | C |

| | | | | | | | | | | |
|---|---|---|---|---|---|---|---|---|---|---|
| ATOM | 1345 | CE1 | PHE A | 564 | 5.247 | 11.848 | 83.996 | 1.00 | 24.83 | C |

| | | | | | | | | | | | |
|---|---|---|---|---|---|---|---|---|---|---|---|
| ANISOU | 1345 | CE1 | PHE A | 564 | 3137 | 3070 | 3228 | −218 | 54 | 9 | C |

| | | | | | | | | | | |
|---|---|---|---|---|---|---|---|---|---|---|
| ATOM | 1346 | CE2 | PHE A | 564 | 6.862 | 13.391 | 83.236 | 1.00 | 25.49 | C |

| | | | | | | | | | | | |
|---|---|---|---|---|---|---|---|---|---|---|---|
| ANISOU | 1346 | CE2 | PHE A | 564 | 3178 | 3121 | 3387 | −210 | 130 | 21 | C |

| | | | | | | | | | | |
|---|---|---|---|---|---|---|---|---|---|---|
| ATOM | 1347 | CZ | PHE A | 564 | 6.346 | 12.084 | 83.205 | 1.00 | 25.85 | C |

| | | | | | | | | | | | |
|---|---|---|---|---|---|---|---|---|---|---|---|
| ANISOU | 1347 | CZ | PHE A | 564 | 3282 | 3142 | 3396 | −227 | 72 | 15 | C |

| | | | | | | | | | | |
|---|---|---|---|---|---|---|---|---|---|---|
| ATOM | 1348 | N | ASP A | 565 | 4.894 | 16.155 | 88.717 | 1.00 | 27.01 | N |

| | | | | | | | | | | | |
|---|---|---|---|---|---|---|---|---|---|---|---|
| ANISOU | 1348 | N | ASP A | 565 | 3369 | 3467 | 3428 | −112 | 231 | 327 | N |

| | | | | | | | | | | |
|---|---|---|---|---|---|---|---|---|---|---|
| ATOM | 1349 | CA | ASP A | 565 | 5.046 | 17.272 | 89.628 | 1.00 | 30.49 | C |

| | | | | | | | | | | | |
|---|---|---|---|---|---|---|---|---|---|---|---|
| ANISOU | 1349 | CA | ASP A | 565 | 3846 | 3764 | 3974 | −168 | 262 | 14 | C |

| | | | | | | | | | | |
|---|---|---|---|---|---|---|---|---|---|---|
| ATOM | 1350 | C | ASP A | 565 | 5.778 | 18.401 | 88.872 | 1.00 | 30.83 | C |

| | | | | | | | | | | | |
|---|---|---|---|---|---|---|---|---|---|---|---|
| ANISOU | 1350 | C | ASP A | 565 | 3881 | 3829 | 4005 | −90 | 262 | 104 | C |

| | | | | | | | | | | |
|---|---|---|---|---|---|---|---|---|---|---|
| ATOM | 1351 | O | ASP A | 565 | 6.840 | 18.072 | 88.319 | 1.00 | 31.71 | O |

| | | | | | | | | | | | |
|---|---|---|---|---|---|---|---|---|---|---|---|
| ANISOU | 1351 | O | ASP A | 565 | 4021 | 3723 | 4302 | −125 | 478 | 1 | O |

| | | | | | | | | | | |
|---|---|---|---|---|---|---|---|---|---|---|
| ATOM | 1352 | CB | ASP A | 565 | 5.877 | 16.880 | 90.853 | 1.00 | 34.44 | C |

| | | | | | | | | | | | |
|---|---|---|---|---|---|---|---|---|---|---|---|
| ANISOU | 1352 | CB | ASP A | 565 | 4294 | 4408 | 4386 | −13 | 15 | 135 | C |

| | | | | | | | | | | |
|---|---|---|---|---|---|---|---|---|---|---|
| ATOM | 1353 | CG | ASP A | 565 | 5.699 | 17.779 | 92.064 | 1.00 | 37.62 | C |

| | | | | | | | | | | | |
|---|---|---|---|---|---|---|---|---|---|---|---|
| ANISOU | 1353 | CG | ASP A | 565 | 4837 | 4805 | 4652 | −46 | 71 | −62 | C |

| | | | | | | | | | | |
|---|---|---|---|---|---|---|---|---|---|---|
| ATOM | 1354 | OD1 | ASP A | 565 | 4.717 | 18.577 | 92.121 | 1.00 | 40.24 | O |

| | | | | | | | | | | | |
|---|---|---|---|---|---|---|---|---|---|---|---|
| ANISOU | 1354 | OD1 | ASP A | 565 | 5238 | 4996 | 5054 | 143 | 156 | −92 | O |

| | | | | | | | | | | |
|---|---|---|---|---|---|---|---|---|---|---|
| ATOM | 1355 | OD2 | ASP A | 565 | 6.516 | 17.728 | 93.003 | 1.00 | 38.63 | O |

| | | | | | | | | | | | |
|---|---|---|---|---|---|---|---|---|---|---|---|
| ANISOU | 1355 | OD2 | ASP A | 565 | 4933 | 4931 | 4814 | −107 | 8 | −1 | O |

| | | | | | | | | | | |
|---|---|---|---|---|---|---|---|---|---|---|
| ATOM | 1356 | N | LEU A | 566 | 5.202 | 19.595 | 88.854 | 1.00 | 29.98 | N |

| | | | | | | | | | | | |
|---|---|---|---|---|---|---|---|---|---|---|---|
| ANISOU | 1356 | N | LEU A | 566 | 3876 | 3820 | 3693 | −106 | 276 | 60 | N |

| | | | | | | | | | | |
|---|---|---|---|---|---|---|---|---|---|---|
| ATOM | 1357 | CA | LEU A | 566 | 5.817 | 20.740 | 88.179 | 1.00 | 30.69 | C |

| | | | | | | | | | | | |
|---|---|---|---|---|---|---|---|---|---|---|---|
| ANISOU | 1357 | CA | LEU A | 566 | 3846 | 3857 | 3959 | −155 | 273 | 54 | C |

| | | | | | | | | | | |
|---|---|---|---|---|---|---|---|---|---|---|
| ATOM | 1358 | C | LEU A | 566 | 6.625 | 21.511 | 89.207 | 1.00 | 31.32 | C |

| | | | | | | | | | | | |
|---|---|---|---|---|---|---|---|---|---|---|---|
| ANISOU | 1358 | C | LEU A | 566 | 3889 | 3974 | 4039 | −175 | 302 | −41 | C |

| | | | | | | | | | | |
|---|---|---|---|---|---|---|---|---|---|---|
| ATOM | 1359 | O | LEU A | 566 | 6.082 | 21.906 | 90.256 | 1.00 | 31.88 | O |

| | | | | | | | | | | | |
|---|---|---|---|---|---|---|---|---|---|---|---|
| ANISOU | 1359 | O | LEU A | 566 | 4061 | 3950 | 4101 | −311 | 440 | −145 | O |

| | | | | | | | | | | |
|---|---|---|---|---|---|---|---|---|---|---|
| ATOM | 1360 | CB | LEU A | 566 | 4.771 | 21.619 | 87.510 | 1.00 | 32.50 | C |

| | | | | | | | | | | | |
|---|---|---|---|---|---|---|---|---|---|---|---|
| ANISOU | 1360 | CB | LEU A | 566 | 4165 | 4051 | 4133 | −30 | 140 | 86 | C |

| | | | | | | | | | | |
|---|---|---|---|---|---|---|---|---|---|---|
| ATOM | 1361 | CG | LEU A | 566 | 3.753 | 20.999 | 86.573 | 1.00 | 32.34 | C |

| | | | | | | | | | | | |
|---|---|---|---|---|---|---|---|---|---|---|---|
| ANISOU | 1361 | CG | LEU A | 566 | 4113 | 3941 | 4233 | −74 | 147 | 37 | C |

| | | | | | | | | | | |
|---|---|---|---|---|---|---|---|---|---|---|
| ATOM | 1362 | OD1 | LEU A | 566 | 2.804 | 21.979 | 85.892 | 1.00 | 34.10 | C |

| | | | | | | | | | | | |
|---|---|---|---|---|---|---|---|---|---|---|---|
| ANISOU | 1362 | OD1 | LEU A | 566 | 4423 | 4203 | 4330 | 2 | 96 | 117 | C |

| | | | | | | | | | | |
|---|---|---|---|---|---|---|---|---|---|---|
| ATOM | 1363 | CD2 | LEU A | 566 | 4.410 | 20.150 | 85.495 | 1.00 | 33.68 | C |

| | | | | | | | | | | | |
|---|---|---|---|---|---|---|---|---|---|---|---|
| ANISOU | 1363 | CD2 | LEU A | 566 | 4321 | 4277 | 4201 | −5 | 160 | −15 | C |

| | | | | | | | | | | |
|---|---|---|---|---|---|---|---|---|---|---|
| ATOM | 1364 | N | ILE A | 567 | 7.932 | 21.719 | 88.987 | 1.00 | 28.81 | N |

| | | | | | | | | | | | |
|---|---|---|---|---|---|---|---|---|---|---|---|
| ANISOU | 1364 | N | ILE A | 567 | 3728 | 3373 | 3848 | −51 | 290 | 95 | N |

| | | | | | | | | | | |
|---|---|---|---|---|---|---|---|---|---|---|
| ATOM | 1365 | CA | ILE A | 567 | 8.763 | 22.332 | 90.009 | 1.00 | 28.23 | C |

| | | | | | | | | | | | |
|---|---|---|---|---|---|---|---|---|---|---|---|
| ANISOU | 1365 | CA | ILE A | 567 | 3572 | 3500 | 3657 | −99 | 358 | 153 | C |

| | | | | | | | | | | |
|---|---|---|---|---|---|---|---|---|---|---|
| ATOM | 1366 | C | ILE A | 567 | 9.680 | 23.441 | 89.482 | 1.00 | 26.45 | C |

| | | | | | | | | | | | |
|---|---|---|---|---|---|---|---|---|---|---|---|
| ANISOU | 1366 | C | ILE A | 567 | 3392 | 3251 | 3409 | −13 | 152 | 101 | C |

| | | | | | | | | | | |
|---|---|---|---|---|---|---|---|---|---|---|
| ATOM | 1367 | O | ILE A | 567 | 10.130 | 23.274 | 88.350 | 1.00 | 25.65 | O |

| | | | | | | | | | | | |
|---|---|---|---|---|---|---|---|---|---|---|---|
| ANISOU | 1367 | O | ILE A | 567 | 3337 | 2913 | 3496 | 49 | 247 | 121 | O |

| | | | | | | | | | | |
|---|---|---|---|---|---|---|---|---|---|---|
| ATOM | 1368 | CB | ILE A | 567 | 9.629 | 21.266 | 90.701 | 1.00 | 30.86 | C |

| | | | | | | | | | | | |
|---|---|---|---|---|---|---|---|---|---|---|---|
| ANISOU | 1368 | CB | ILE A | 567 | 3950 | 3828 | 3949 | −20 | 158 | 286 | C |

| | | | | | | | | | | |
|---|---|---|---|---|---|---|---|---|---|---|
| ATOM | 1369 | CG1 | ILE A | 567 | 10.558 | 21.854 | 91.768 | 1.00 | 32.95 | C |

| | | | | | | | | | | | |
|---|---|---|---|---|---|---|---|---|---|---|---|
| ANISOU | 1369 | CG1 | ILE A | 567 | 4180 | 4044 | 4296 | −87 | −11 | 174 | C |

| | | | | | | | | | | |
|---|---|---|---|---|---|---|---|---|---|---|
| ATOM | 1370 | CG2 | ILE A | 567 | 10.493 | 20.436 | 89.775 | 1.00 | 33.22 | C |

| | | | | | | | | | | | |
|---|---|---|---|---|---|---|---|---|---|---|---|
| ANISOU | 1370 | CD2 | ILE A | 567 | 4314 | 4161 | 4146 | 40 | 183 | 98 | C |

| | | | | | | | | | | |
|---|---|---|---|---|---|---|---|---|---|---|
| ATOM | 1371 | CD1 | ILE A | 567 | 9.803 | 22.334 | 92.986 | 1.00 | 35.67 | C |

| | | | | | | | | | | | |
|---|---|---|---|---|---|---|---|---|---|---|---|
| ANISOU | 1371 | CD1 | ILE A | 567 | 4647 | 4518 | 4388 | −50 | 78 | 43 | C |

| | | | | | | | | | | |
|---|---|---|---|---|---|---|---|---|---|---|
| ATOM | 1372 | N | HIS A | 568 | 9.807 | 24.516 | 90.224 | 1.00 | 24.84 | N |

| | | | | | | | | | | | |
|---|---|---|---|---|---|---|---|---|---|---|---|
| ANISOU | 1372 | N | HIS A | 568 | 3102 | 3302 | 3033 | −61 | 145 | 208 | N |

| | | | | | | | | | | |
|---|---|---|---|---|---|---|---|---|---|---|
| ATOM | 1373 | CA | HIS A | 568 | 10.759 | 25.581 | 89.879 | 1.00 | 25.70 | C |

| | | | | | | | | | | | |
|---|---|---|---|---|---|---|---|---|---|---|---|
| ANISOU | 1373 | CA | HIS A | 568 | 3155 | 3457 | 3152 | −146 | 39 | 134 | C |

| | | | | | | | | | | |
|---|---|---|---|---|---|---|---|---|---|---|
| ATOM | 1374 | C | HIS A | 568 | 12.034 | 25.130 | 90.629 | 1.00 | 26.73 | C |

| | | | | | | | | | | | |
|---|---|---|---|---|---|---|---|---|---|---|---|
| ANISOU | 1374 | C | HIS A | 568 | 3310 | 3625 | 3222 | 57 | 66 | 117 | C |

| | | | | | | | | | | |
|---|---|---|---|---|---|---|---|---|---|---|
| ATOM | 1375 | O | HIS A | 568 | 12.050 | 25.213 | 91.863 | 1.00 | 27.81 | O |

| | | | | | | | | | | | |
|---|---|---|---|---|---|---|---|---|---|---|---|
| ANISOU | 1375 | O | HIS A | 568 | 3427 | 3972 | 3166 | −24 | −6 | 196 | O |

| | | | | | | | | | | |
|---|---|---|---|---|---|---|---|---|---|---|
| ATOM | 1376 | CB | HIS A | 568 | 10.274 | 26.945 | 90.324 | 1.00 | 27.99 | C |

| | | | | | | | | | | | |
|---|---|---|---|---|---|---|---|---|---|---|---|
| ANISOU | 1376 | CB | HIS A | 568 | 3593 | 3575 | 3466 | −43 | 52 | 27 | C |

| | | | | | | | | | | |
|---|---|---|---|---|---|---|---|---|---|---|
| ATOM | 1377 | CG | HIS A | 568 | 11.100 | 28.126 | 89.865 | 1.00 | 31.14 | C |

| | | | | | | | | | | | |
|---|---|---|---|---|---|---|---|---|---|---|---|
| ANISOU | 1377 | CG | HIS A | 568 | 4036 | 3871 | 3925 | −196 | 95 | 52 | C |

| | | | | | | | | | | |
|---|---|---|---|---|---|---|---|---|---|---|
| ATOM | 1378 | ND1 | HIS A | 568 | 10.560 | 29.082 | 89.037 | 1.00 | 31.70 | N |

| | | | | | | | | | | | |
|---|---|---|---|---|---|---|---|---|---|---|---|
| ANISOU | 1378 | ND1 | HIS A | 568 | 4013 | 3873 | 4159 | 46 | 146 | 11 | N |

| | | | | | | | | | | |
|---|---|---|---|---|---|---|---|---|---|---|
| ATOM | 1379 | CD2 | HIS A | 568 | 12.379 | 28.514 | 90.071 | 1.00 | 33.15 | C |

| | | | | | | | | | | | |
|---|---|---|---|---|---|---|---|---|---|---|---|
| ANISOU | 1379 | CD2 | HIS A | 568 | 4084 | 4198 | 4315 | −162 | 39 | 39 | C |

| | | | | | | | | | | |
|---|---|---|---|---|---|---|---|---|---|---|
| ATOM | 1380 | CE1 | HIS A | 568 | 11.485 | 30.007 | 88.750 | 1.00 | 32.74 | C |

| | | | | | | | | | | | |
|---|---|---|---|---|---|---|---|---|---|---|---|
| ANISOU | 1380 | CE1 | HIS A | 568 | 4265 | 3912 | 4262 | −44 | 10 | −53 | C |

| | | | | | | | | | | |
|---|---|---|---|---|---|---|---|---|---|---|
| ATOM | 1381 | NE2 | HIS A | 568 | 12.590 | 29.695 | 89.372 | 1.00 | 32.32 | N |

| | | | | | | | | | | | |
|---|---|---|---|---|---|---|---|---|---|---|---|
| ANISOU | 1381 | NE2 | HIS A | 568 | 4113 | 3897 | 4272 | −26 | 19 | −126 | N |

| | | | | | | | | | | |
|---|---|---|---|---|---|---|---|---|---|---|
| ATOM | 1382 | N | LEU A | 569 | 12.987 | 24.543 | 89.901 | 1.00 | 25.62 | N |

| | | | | | | | | | | | |
|---|---|---|---|---|---|---|---|---|---|---|---|
| ANISOU | 1382 | N | LEU A | 569 | 3271 | 3325 | 3140 | −43 | 239 | 315 | N |

| | | | | | | | | | | |
|---|---|---|---|---|---|---|---|---|---|---|
| ATOM | 1383 | CA | LEU A | 569 | 14.184 | 23.990 | 90.507 | 1.00 | 27.08 | C |

| | | | | | | | | | | | |
|---|---|---|---|---|---|---|---|---|---|---|---|
| ANISOU | 1383 | CA | LEU A | 569 | 3364 | 3567 | 3360 | −6 | 133 | 217 | C |

| | | | | | | | | | | |
|---|---|---|---|---|---|---|---|---|---|---|
| ATOM | 1384 | C | LEU A | 569 | 15.212 | 25.016 | 90.919 | 1.00 | 28.36 | C |

| | | | | | | | | | | | |
|---|---|---|---|---|---|---|---|---|---|---|---|
| ANISOU | 1384 | C | LEU A | 569 | 3513 | 3742 | 3521 | −60 | 36 | 187 | C |

| | | | | | | | | | | |
|---|---|---|---|---|---|---|---|---|---|---|
| ATOM | 1385 | O | LEU A | 569 | 15.493 | 25.954 | 90.149 | 1.00 | 29.04 | O |

| | | | | | | | | | | | |
|---|---|---|---|---|---|---|---|---|---|---|---|
| ANISOU | 1385 | O | LEU A | 569 | 3578 | 3840 | 3615 | −195 | 68 | 312 | O |

| | | | | | | | | | | |
|---|---|---|---|---|---|---|---|---|---|---|
| ATOM | 1386 | CB | LEU A | 569 | 14.881 | 23.040 | 89.496 | 1.00 | 28.77 | C |

| | | | | | | | | | | | |
|---|---|---|---|---|---|---|---|---|---|---|---|
| ANISOU | 1386 | CB | LEU A | 569 | 3662 | 3640 | 3628 | 70 | 193 | 103 | C |

| | | | | | | | | | | |
|---|---|---|---|---|---|---|---|---|---|---|
| ATOM | 1387 | CG | LEU A | 569 | 14.059 | 21.884 | 88.940 | 1.00 | 31.08 | C |

| | | | | | | | | | | | |
|---|---|---|---|---|---|---|---|---|---|---|---|
| ANISOU | 1387 | CG | LEU A | 569 | 3961 | 3909 | 3940 | −25 | 110 | −85 | C |

| | | | | | | | | | | |
|---|---|---|---|---|---|---|---|---|---|---|
| ATOM | 1388 | CD1 | LEU A | 569 | 14.730 | 21.287 | 87.700 | 1.00 | 30.23 | C |

| | | | | | | | | | | | |
|---|---|---|---|---|---|---|---|---|---|---|---|
| ANISOU | 1388 | CG1 | LEU A | 569 | 3875 | 3692 | 3918 | −64 | 86 | −16 | C |

| | | | | | | | | | | |
|---|---|---|---|---|---|---|---|---|---|---|
| ATOM | 1389 | CD2 | LEU A | 569 | 13.837 | 20.810 | 90.005 | 1.00 | 32.36 | C |

| | | | | | | | | | | | |
|---|---|---|---|---|---|---|---|---|---|---|---|
| ANISOU | 1389 | CD2 | LEU A | 569 | 4118 | 3988 | 4190 | 120 | 86 | 72 | C |

| | | | | | | | | | | |
|---|---|---|---|---|---|---|---|---|---|---|
| ATOM | 1390 | N | ASP A | 570 | 15.805 | 24.873 | 92.082 | 1.00 | 28.26 | N |

| | | | | | | | | | | | |
|---|---|---|---|---|---|---|---|---|---|---|---|
| ANISOU | 1390 | N | ASP A | 570 | 3498 | 3877 | 3362 | 1 | 239 | 274 | N |

| | | | | | | | | | | |
|---|---|---|---|---|---|---|---|---|---|---|
| ATOM | 1391 | CA | ASP A | 570 | 16.844 | 25.760 | 92.553 | 1.00 | 29.70 | C |

| | | | | | | | | | | | |
|---|---|---|---|---|---|---|---|---|---|---|---|
| ANISOU | 1391 | CA | ASP A | 570 | 3647 | 3905 | 3733 | −38 | 194 | 88 | C |

| | | | | | | | | | | |
|---|---|---|---|---|---|---|---|---|---|---|
| ATOM | 1392 | C | ASP A | 570 | 18.160 | 25.495 | 91.825 | 1.00 | 26.46 | C |

| | | | | | | | | | | | |
|---|---|---|---|---|---|---|---|---|---|---|---|
| ANISOU | 1392 | C | ASP A | 570 | 3306 | 3347 | 3401 | −55 | −19 | 182 | C |

| | | | | | | | | | | |
|---|---|---|---|---|---|---|---|---|---|---|
| ATOM | 1393 | O | ASP A | 570 | 18.451 | 24.337 | 91.609 | 1.00 | 25.67 | O |

| | | | | | | | | | | | |
|---|---|---|---|---|---|---|---|---|---|---|---|
| ANISOU | 1393 | O | ASP A | 570 | 3032 | 3198 | 3522 | −383 | 422 | 424 | O |

| | | | | | | | | | | |
|---|---|---|---|---|---|---|---|---|---|---|
| ATOM | 1394 | CB | ASP A | 570 | 17.215 | 25.391 | 94.013 | 1.00 | 34.38 | C |

| | | | | | | | | | | | |
|---|---|---|---|---|---|---|---|---|---|---|---|
| ANISOU | 1394 | CB | ASP A | 570 | 4400 | 4612 | 4052 | 6 | −90 | 179 | C |

| | | | | | | | | | | |
|---|---|---|---|---|---|---|---|---|---|---|
| ATOM | 1395 | CG | ASP A | 570 | 16.044 | 25.734 | 94.911 | 1.00 | 37.90 | C |

| | | | | | | | | | | | |
|---|---|---|---|---|---|---|---|---|---|---|---|
| ANISOU | 1395 | CG | ASP A | 570 | 4636 | 5086 | 4680 | 44 | 129 | −33 | C |

| | | | | | | | | | | |
|---|---|---|---|---|---|---|---|---|---|---|
| ATOM | 1396 | OD1 | ASP A | 570 | 15.544 | 26.859 | 94.700 | 1.00 | 40.78 | O |

| | | | | | | | | | | | |
|---|---|---|---|---|---|---|---|---|---|---|---|
| ANISOU | 1396 | OD1 | ASP A | 570 | 5161 | 5227 | 5107 | 130 | 22 | 32 | O |

| | | | | | | | | | | |
|---|---|---|---|---|---|---|---|---|---|---|
| ATOM | 1397 | OD2 | ASP A | 570 | 15.736 | 24.842 | 95.727 | 1.00 | 39.89 | O |

| | | | | | | | | | | | |
|---|---|---|---|---|---|---|---|---|---|---|---|
| ANISOU | 1397 | OD2 | ASP A | 570 | 4875 | 5415 | 4867 | 24 | 160 | 151 | O |

| | | | | | | | | | | |
|---|---|---|---|---|---|---|---|---|---|---|
| ATOM | 1398 | N | CYS A | 571 | 18.994 | 26.481 | 91.568 | 1.00 | 25.36 | N |

| | | | | | | | | | | | |
|---|---|---|---|---|---|---|---|---|---|---|---|
| ANISOU | 1398 | N | CYS A | 571 | 3286 | 3138 | 3213 | 61 | 32 | 321 | N |

| | | | | | | | | | | |
|---|---|---|---|---|---|---|---|---|---|---|
| ATOM | 1399 | CA | CYS A | 571 | 20.318 | 26.223 | 91.010 | 1.00 | 23.70 | C |

| | | | | | | | | | | | |
|---|---|---|---|---|---|---|---|---|---|---|---|
| ANISOU | 1399 | CA | CYS A | 571 | 3352 | 2818 | 2836 | 49 | 91 | 50 | C |

| | | | | | | | | | | |
|---|---|---|---|---|---|---|---|---|---|---|
| ATOM | 1400 | C | CYS A | 571 | 21.241 | 27.380 | 91.342 | 1.00 | 23.46 | C |

| | | | | | | | | | | | |
|---|---|---|---|---|---|---|---|---|---|---|---|
| ANISOU | 1400 | C | CYS A | 571 | 3208 | 2812 | 2892 | 28 | 171 | 138 | C |

| | | | | | | | | | | |
|---|---|---|---|---|---|---|---|---|---|---|
| ATOM | 1401 | O | CYS A | 571 | 20.748 | 28.429 | 91.748 | 1.00 | 23.13 | O |

| | | | | | | | | | | | |
|---|---|---|---|---|---|---|---|---|---|---|---|
| ANISOU | 1401 | O | CYS A | 571 | 3183 | 2724 | 2883 | −24 | 310 | 220 | O |

| | | | | | | | | | | |
|---|---|---|---|---|---|---|---|---|---|---|
| ATOM | 1402 | CB | CYS A | 571 | 20.278 | 25.968 | 89.475 | 1.00 | 22.21 | C |

| | | | | | | | | | | | |
|---|---|---|---|---|---|---|---|---|---|---|---|
| ANISOU | 1402 | CB | CYS A | 571 | 3231 | 2509 | 2699 | 171 | 148 | 210 | C |

| | | | | | | | | | | |
|---|---|---|---|---|---|---|---|---|---|---|
| ATOM | 1403 | SG | CYS A | 571 | 20.045 | 27.501 | 88.538 | 1.00 | 21.88 | S |

| | | | | | | | | | | | |
|---|---|---|---|---|---|---|---|---|---|---|---|
| ANISOU | 1403 | SG | CYS A | 571 | 3662 | 2459 | 2192 | 96 | 372 | 113 | S |

| | | | | | | | | | | |
|---|---|---|---|---|---|---|---|---|---|---|
| ATOM | 1404 | N | PRO A | 572 | 22.548 | 27.222 | 91.217 | 1.00 | 24.54 | N |

| | | | | | | | | | | | |
|---|---|---|---|---|---|---|---|---|---|---|---|
| ANISOU | 1404 | N | PRO A | 572 | 3200 | 2947 | 3176 | 7 | 178 | 40 | N |

| | | | | | | | | | | |
|---|---|---|---|---|---|---|---|---|---|---|
| ATOM | 1405 | CA | PRO A | 572 | 23.518 | 28.285 | 91.440 | 1.00 | 24.11 | C |

| | | | | | | | | | | | |
|---|---|---|---|---|---|---|---|---|---|---|---|
| ANISOU | 1405 | CA | PRO A | 572 | 3218 | 2836 | 3106 | 56 | 174 | 81 | C |

| | | | | | | | | | | |
|---|---|---|---|---|---|---|---|---|---|---|
| ATOM | 1406 | C | PRO A | 572 | 23.171 | 29.463 | 90.534 | 1.00 | 23.17 | C |

| | | | | | | | | | | | |
|---|---|---|---|---|---|---|---|---|---|---|---|
| ANISOU | 1406 | C | PRO A | 572 | 3137 | 2779 | 2886 | −51 | 148 | 16 | C |

| | | | | | | | | | | |
|---|---|---|---|---|---|---|---|---|---|---|
| ATOM | 1407 | O | PRO A | 572 | 22.841 | 29.259 | 89.382 | 1.00 | 22.63 | O |

| | | | | | | | | | | | |
|---|---|---|---|---|---|---|---|---|---|---|---|
| ANISOU | 1407 | O | PRO A | 572 | 3190 | 2682 | 2727 | −133 | 307 | 149 | O |

| | | | | | | | | | | |
|---|---|---|---|---|---|---|---|---|---|---|
| ATOM | 1408 | CB | PRO A | 572 | 24.876 | 27.683 | 91.122 | 1.00 | 25.09 | C |

| | | | | | | | | | | | |
|---|---|---|---|---|---|---|---|---|---|---|---|
| ANISOU | 1408 | CB | PRO A | 572 | 3137 | 3059 | 3335 | 79 | 110 | 88 | C |

| | | | | | | | | | | |
|---|---|---|---|---|---|---|---|---|---|---|
| ATOM | 1409 | CG | PRO A | 572 | 24.643 | 26.190 | 91.232 | 1.00 | 26.29 | C |

| | | | | | | | | | | | |
|---|---|---|---|---|---|---|---|---|---|---|---|
| ANISOU | 1409 | CG | PRO A | 572 | 3405 | 3154 | 3429 | −151 | 50 | 17 | C |

| | | | | | | | | | | |
|---|---|---|---|---|---|---|---|---|---|---|
| ATOM | 1410 | CD | PRO A | 572 | 23.218 | 25.967 | 90.760 | 1.00 | 24.99 | C |

| | | | | | | | | | | | |
|---|---|---|---|---|---|---|---|---|---|---|---|
| ANISOU | 1410 | CD | PRO A | 572 | 3202 | 2978 | 3315 | 44 | 151 | 87 | C |

| | | | | | | | | | | |
|---|---|---|---|---|---|---|---|---|---|---|
| ATOM | 1411 | N | GLN A | 573 | 23.191 | 30.663 | 91.113 | 1.00 | 21.01 | N |

| | | | | | | | | | | | |
|---|---|---|---|---|---|---|---|---|---|---|---|
| ANISOU | 1411 | N | GLN A | 573 | 3038 | 2644 | 2303 | −81 | 295 | 134 | N |

| | | | | | | | | | | |
|---|---|---|---|---|---|---|---|---|---|---|
| ATOM | 1412 | CA | GLN A | 573 | 22.731 | 31.830 | 90.337 | 1.00 | 21.31 | C |

| | | | | | | | | | | | |
|---|---|---|---|---|---|---|---|---|---|---|---|
| ANISOU | 1412 | CA | GLN A | 573 | 2952 | 2581 | 2562 | 25 | 191 | 48 | C |

| | | | | | | | | | | |
|---|---|---|---|---|---|---|---|---|---|---|
| ATOM | 1413 | C | GLN A | 573 | 23.958 | 32.538 | 89.749 | 1.00 | 21.12 | C |

| | | | | | | | | | | | |
|---|---|---|---|---|---|---|---|---|---|---|---|
| ANISOU | 1413 | C | GLN A | 573 | 2861 | 2722 | 2442 | −37 | 166 | 16 | C |

| | | | | | | | | | | |
|---|---|---|---|---|---|---|---|---|---|---|
| ATOM | 1414 | O | GLN A | 573 | 25.010 | 32.581 | 90.392 | 1.00 | 22.44 | O |

| | | | | | | | | | | | |
|---|---|---|---|---|---|---|---|---|---|---|---|
| ANISOU | 1414 | O | GLN A | 573 | 3115 | 2770 | 2641 | −192 | 111 | 93 | O |

| | | | | | | | | | | |
|---|---|---|---|---|---|---|---|---|---|---|
| ATOM | 1415 | CB | GLN A | 573 | 22.020 | 32.789 | 91.312 | 1.00 | 21.04 | C |

| | | | | | | | | | | | |
|---|---|---|---|---|---|---|---|---|---|---|---|
| ANISOU | 1415 | CB | GLN A | 573 | 2833 | 2769 | 2391 | −95 | 130 | −104 | C |

| | | | | | | | | | | |
|---|---|---|---|---|---|---|---|---|---|---|
| ATOM | 1416 | CG | GLN A | 573 | 20.721 | 32.169 | 91.876 | 1.00 | 21.77 | C |

| | | | | | | | | | | | |
|---|---|---|---|---|---|---|---|---|---|---|---|
| ANISOU | 1416 | CG | GLN A | 573 | 2805 | 2889 | 2578 | 26 | 239 | −49 | C |

| | | | | | | | | | | |
|---|---|---|---|---|---|---|---|---|---|---|
| ATOM | 1417 | CD | GLN A | 573 | 19.661 | 31.965 | 90.827 | 1.00 | 23.08 | C |

| | | | | | | | | | | | |
|---|---|---|---|---|---|---|---|---|---|---|---|
| ANISOU | 1417 | CD | GLN A | 573 | 3171 | 2986 | 2614 | −15 | 180 | −41 | C |

| | | | | | | | | | | |
|---|---|---|---|---|---|---|---|---|---|---|
| ATOM | 1418 | OE1 | GLN A | 573 | 19.046 | 32.951 | 90.356 | 1.00 | 23.03 | O |

| | | | | | | | | | | | |
|---|---|---|---|---|---|---|---|---|---|---|---|
| ANISOU | 1418 | OE1 | GLN A | 573 | 3086 | 3003 | 2660 | 125 | 314 | −10 | O |

| | | | | | | | | | | |
|---|---|---|---|---|---|---|---|---|---|---|
| ATOM | 1419 | NE2 | GLN A | 573 | 19.416 | 30.727 | 90.379 | 1.00 | 22.52 | N |

| | | | | | | | | | | | |
|---|---|---|---|---|---|---|---|---|---|---|---|
| ANISOU | 1419 | NE2 | GLN A | 573 | 3255 | 2844 | 2459 | 55 | 306 | 49 | N |

| | | | | | | | | | | |
|---|---|---|---|---|---|---|---|---|---|---|
| ATOM | 1420 | N | GLN A | 574 | 23.912 | 33.039 | 88.526 | 1.00 | 20.37 | N |

| | | | | | | | | | | | |
|---|---|---|---|---|---|---|---|---|---|---|---|
| ANISOU | 1420 | N | GLN A | 574 | 2923 | 2289 | 2526 | −162 | 125 | 126 | N |

| | | | | | | | | | | |
|---|---|---|---|---|---|---|---|---|---|---|
| ATOM | 1421 | CA | GLN A | 574 | 24.967 | 33.723 | 87.887 | 1.00 | 21.00 | C |

| | | | | | | | | | | | |
|---|---|---|---|---|---|---|---|---|---|---|---|
| ANISOU | 1421 | CA | GLN A | 574 | 2788 | 2536 | 2655 | −225 | 48 | −26 | C |

| | | | | | | | | | | |
|---|---|---|---|---|---|---|---|---|---|---|
| ATOM | 1422 | C | GLN A | 574 | 25.221 | 35.048 | 88.589 | 1.00 | 21.03 | C |

| | | | | | | | | | | | |
|---|---|---|---|---|---|---|---|---|---|---|---|
| ANISOU | 1422 | C | GLN A | 574 | 2803 | 2462 | 2728 | −96 | 115 | −40 | C |

| | | | | | | | | | | |
|---|---|---|---|---|---|---|---|---|---|---|
| ATOM | 1423 | O | GLN A | 574 | 24.270 | 35.792 | 98.897 | 1.00 | 20.78 | O |

| | | | | | | | | | | | |
|---|---|---|---|---|---|---|---|---|---|---|---|
| ANISOU | 1423 | O | GLN A | 574 | 2756 | 2377 | 2764 | −203 | 51 | −265 | O |

| | | | | | | | | | | |
|---|---|---|---|---|---|---|---|---|---|---|
| ATOM | 1424 | CB | GLN A | 574 | 24.584 | 33.888 | 86.406 | 1.00 | 21.23 | C |

| | | | | | | | | | | | |
|---|---|---|---|---|---|---|---|---|---|---|---|
| ANISOU | 1424 | CB | GLN A | 574 | 2863 | 2623 | 2581 | −24 | 279 | 155 | C |

| | | | | | | | | | | |
|---|---|---|---|---|---|---|---|---|---|---|
| ATOM | 1425 | CG | GLN A | 574 | 23.446 | 34.893 | 86.183 | 1.00 | 22.92 | C |

| | | | | | | | | | | | |
|---|---|---|---|---|---|---|---|---|---|---|---|
| ANISOU | 1425 | CG | GLN A | 574 | 3015 | 2804 | 2888 | −35 | 38 | 62 | C |

| | | | | | | | | | | |
|---|---|---|---|---|---|---|---|---|---|---|
| ATOM | 1426 | CD | GLN A | 574 | 22.949 | 34.783 | 84.758 | 1.00 | 23.64 | C |

| | | | | | | | | | | | |
|---|---|---|---|---|---|---|---|---|---|---|---|
| ANISOU | 1426 | CD | GLN A | 574 | 3062 | 3021 | 2900 | −95 | 34 | 91 | C |

| | | | | | | | | | | |
|---|---|---|---|---|---|---|---|---|---|---|
| ATOM | 1427 | OE1 | GLN A | 574 | 22.371 | 33.774 | 94.366 | 1.00 | 23.51 | O |

| | | | | | | | | | | | |
|---|---|---|---|---|---|---|---|---|---|---|---|
| ANISOU | 1427 | OE1 | GLN A | 574 | 3291 | 2898 | 2742 | −59 | 107 | 119 | O |

| | | | | | | | | | | |
|---|---|---|---|---|---|---|---|---|---|---|
| ATOM | 1428 | NE2 | GLN A | 574 | 23.236 | 35.797 | 83.948 | 1.00 | 24.35 | N |

| | | | | | | | | | | | |
|---|---|---|---|---|---|---|---|---|---|---|---|
| ANISOU | 1428 | NE2 | GLN A | 574 | 3349 | 2886 | 3017 | −177 | −65 | 102 | N |

| | | | | | | | | | | |
|---|---|---|---|---|---|---|---|---|---|---|
| ATOM | 1429 | N | PRO A | 575 | 26.489 | 35.441 | 88.705 | 1.00 | 20.01 | N |

| | | | | | | | | | | | |
|---|---|---|---|---|---|---|---|---|---|---|---|
| ANISOU | 1429 | N | PRO A | 575 | 2714 | 2225 | 2664 | −105 | 42 | −50 | N |

| | | | | | | | | | | |
|---|---|---|---|---|---|---|---|---|---|---|
| ATOM | 1430 | CA | PRO A | 575 | 26.856 | 36.702 | 89.356 | 1.00 | 21.59 | C |

| | | | | | | | | | | | |
|---|---|---|---|---|---|---|---|---|---|---|---|
| ANISOU | 1430 | CA | PRO A | 575 | 3074 | 2403 | 2727 | −130 | −50 | −53 | C |

| | | | | | | | | | | |
|---|---|---|---|---|---|---|---|---|---|---|
| ATOM | 1431 | C | PRO A | 575 | 27.025 | 37.844 | 88.371 | 1.00 | 22.37 | C |

| | | | | | | | | | | | |
|---|---|---|---|---|---|---|---|---|---|---|---|
| ANISOU | 1431 | C | PRO A | 575 | 3188 | 2633 | 2678 | −219 | 69 | 11 | C |

| | | | | | | | | | | |
|---|---|---|---|---|---|---|---|---|---|---|
| ATOM | 1432 | O | PRO A | 575 | 27.517 | 38.919 | 88.750 | 1.00 | 25.00 | O |

| | | | | | | | | | | | |
|---|---|---|---|---|---|---|---|---|---|---|---|
| ANISOU | 1432 | O | PRO A | 575 | 3881 | 2758 | 2858 | −423 | 101 | −59 | O |

| | | | | | | | | | | |
|---|---|---|---|---|---|---|---|---|---|---|
| ATOM | 1433 | CB | PRO A | 575 | 28.211 | 36.325 | 89.960 | 1.00 | 21.25 | C |

| | | | | | | | | | | | |
|---|---|---|---|---|---|---|---|---|---|---|---|
| ANISOU | 1433 | CB | PRO A | 575 | 2817 | 2454 | 2802 | −119 | 79 | −128 | C |

| | | | | | | | | | | |
|---|---|---|---|---|---|---|---|---|---|---|
| ATOM | 1434 | CG | PRO A | 575 | 28.847 | 35.492 | 88.873 | 1.00 | 20.60 | C |

| | | | | | | | | | | | |
|---|---|---|---|---|---|---|---|---|---|---|---|
| ANISOU | 1434 | CG | PRO A | 575 | 2747 | 2276 | 2804 | −76 | 88 | −36 | C |

| | | | | | | | | | | |
|---|---|---|---|---|---|---|---|---|---|---|
| ATOM | 1435 | CD | PRO A | 575 | 27.686 | 34.608 | 88.385 | 1.00 | 21.45 | C |

| | | | | | | | | | | | |
|---|---|---|---|---|---|---|---|---|---|---|---|
| ANISOU | 1435 | CD | PRO A | 575 | 2591 | 2479 | 3080 | −64 | −82 | 69 | C |

| | | | | | | | | | | |
|---|---|---|---|---|---|---|---|---|---|---|
| ATOM | 1436 | N | ASN A | 576 | 26.620 | 37.623 | 87.130 | 1.00 | 21.02 | N |

| | | | | | | | | | | | |
|---|---|---|---|---|---|---|---|---|---|---|---|
| ANISOU | 1436 | N | ASN A | 576 | 3061 | 2327 | 2598 | −112 | 54 | 85 | N |

| | | | | | | | | | | |
|---|---|---|---|---|---|---|---|---|---|---|
| ATOM | 1437 | CA | ASN A | 576 | 26.821 | 38.599 | 86.049 | 1.00 | 19.66 | C |

| | | | | | | | | | | | |
|---|---|---|---|---|---|---|---|---|---|---|---|
| ANISOU | 1437 | CA | ASN A | 576 | 2870 | 2133 | 2467 | −138 | −7 | −31 | C |

| | | | | | | | | | | |
|---|---|---|---|---|---|---|---|---|---|---|
| ATOM | 1438 | C | ASN A | 576 | 25.662 | 38.544 | 85.081 | 1.00 | 19.65 | C |

| | | | | | | | | | | | |
|---|---|---|---|---|---|---|---|---|---|---|---|
| ANISOU | 1438 | C | ASN A | 576 | 2863 | 2157 | 2445 | 106 | 84 | −20 | C |

| | | | | | | | | | | |
|---|---|---|---|---|---|---|---|---|---|---|
| ATOM | 1439 | O | ASN A | 576 | 24.758 | 37.713 | 85.260 | 1.00 | 20.86 | O |

| | | | | | | | | | | | |
|---|---|---|---|---|---|---|---|---|---|---|---|
| ANISOU | 1439 | O | ASN A | 576 | 2867 | 2310 | 2750 | −104 | 72 | −9 | O |

| | | | | | | | | | | |
|---|---|---|---|---|---|---|---|---|---|---|
| ATOM | 1440 | CB | ASN A | 576 | 28.154 | 38.398 | 85.322 | 1.00 | 19.47 | C |

| | | | | | | | | | | | |
|---|---|---|---|---|---|---|---|---|---|---|---|
| ANISOU | 1440 | CB | ASN A | 576 | 2697 | 2189 | 2512 | −95 | −112 | −51 | C |

| | | | | | | | | | | |
|---|---|---|---|---|---|---|---|---|---|---|
| ATOM | 1441 | CG | ASN A | 576 | 28.245 | 36.983 | 84.730 | 1.00 | 20.85 | C |

| | | | | | | | | | | | |
|---|---|---|---|---|---|---|---|---|---|---|---|
| ANISOU | 1441 | CG | ASN A | 576 | 2773 | 2331 | 2817 | −49 | −169 | −107 | C |

| | | | | | | | | | | |
|---|---|---|---|---|---|---|---|---|---|---|
| ATOM | 1442 | OD1 | ASN A | 576 | 27.374 | 36.665 | 83.914 | 1.00 | 19.46 | O |

| | | | | | | | | | | | |
|---|---|---|---|---|---|---|---|---|---|---|---|
| ANISOU | 1442 | OD1 | ASN A | 576 | 3092 | 1840 | 2461 | −30 | −274 | 121 | O |

| | | | | | | | | | | |
|---|---|---|---|---|---|---|---|---|---|---|
| ATOM | 1443 | ND2 | ASN A | 576 | 29.208 | 36.159 | 85.131 | 1.00 | 21.52 | N |

| | | | | | | | | | | | |
|---|---|---|---|---|---|---|---|---|---|---|---|
| ANISOU | 1443 | ND2 | ASN A | 576 | 2989 | 2195 | 2993 | −122 | −90 | 96 | N |

| | | | | | | | | | | |
|---|---|---|---|---|---|---|---|---|---|---|
| ATOM | 1444 | N | GLY A | 577 | 25.706 | 39.455 | 84.077 | 1.00 | 18.58 | N |

| | | | | | | | | | | | |
|---|---|---|---|---|---|---|---|---|---|---|---|
| ANISOU | 1444 | N | GLY A | 577 | 2819 | 1935 | 2307 | 165 | 27 | −114 | N |

| | | | | | | | | | | |
|---|---|---|---|---|---|---|---|---|---|---|
| ATOM | 1445 | CA | GLY A | 577 | 24.568 | 39.415 | 83.156 | 1.00 | 19.56 | C |

| | | | | | | | | | | | |
|---|---|---|---|---|---|---|---|---|---|---|---|
| ANISOU | 1445 | CA | GLY A | 577 | 2721 | 2208 | 2502 | 302 | −5 | −106 | C |

| | | | | | | | | | | |
|---|---|---|---|---|---|---|---|---|---|---|
| ATOM | 1446 | C | GLY A | 577 | 24.797 | 38.665 | 81.855 | 1.00 | 19.64 | C |

| | | | | | | | | | | | |
|---|---|---|---|---|---|---|---|---|---|---|---|
| ANISOU | 1446 | C | GLY A | 577 | 2836 | 2245 | 2383 | 48 | 138 | 15 | C |

| | | | | | | | | | | |
|---|---|---|---|---|---|---|---|---|---|---|
| ATOM | 1447 | O | GLY A | 577 | 23.850 | 38.716 | 81.066 | 1.00 | 22.23 | O |

| | | | | | | | | | | | |
|---|---|---|---|---|---|---|---|---|---|---|---|
| ANISOU | 1447 | O | GLY A | 577 | 3087 | 2518 | 2841 | 255 | −44 | 27 | O |

| | | | | | | | | | | |
|---|---|---|---|---|---|---|---|---|---|---|
| ATOM | 1448 | N | TYR A | 578 | 25.878 | 37.922 | 81.682 | 1.00 | 16.92 | N |

| | | | | | | | | | | | |
|---|---|---|---|---|---|---|---|---|---|---|---|
| ANISOU | 1448 | N | TYR A | 578 | 2714 | 1577 | 2137 | −76 | 165 | 21 | N |

| | | | | | | | | | | |
|---|---|---|---|---|---|---|---|---|---|---|
| ATOM | 1449 | CA | TYR A | 578 | 26.145 | 37.336 | 80.362 | 1.00 | 16.24 | C |

| | | | | | | | | | | | |
|---|---|---|---|---|---|---|---|---|---|---|---|
| ANISOU | 1449 | CA | TYR A | 578 | 2585 | 1616 | 1969 | −66 | 24 | 85 | C |

| | | | | | | | | | | |
|---|---|---|---|---|---|---|---|---|---|---|
| ATOM | 1450 | C | TYR A | 578 | 26.284 | 35.807 | 80.384 | 1.00 | 16.73 | C |

| | | | | | | | | | | | |
|---|---|---|---|---|---|---|---|---|---|---|---|
| ANISOU | 1450 | C | TYR A | 578 | 2584 | 1605 | 2170 | 22 | 28 | −38 | C |

| | | | | | | | | | | |
|---|---|---|---|---|---|---|---|---|---|---|
| ATOM | 1451 | O | TYR A | 578 | 26.277 | 35.191 | 79.311 | 1.00 | 17.44 | O |

| | | | | | | | | | | | |
|---|---|---|---|---|---|---|---|---|---|---|---|
| ANISOU | 1451 | O | TYR A | 578 | 2695 | 1778 | 2152 | −307 | −43 | −34 | O |

| | | | | | | | | | | |
|---|---|---|---|---|---|---|---|---|---|---|
| ATOM | 1452 | CB | TYR A | 578 | 27.464 | 37.881 | 79.819 | 1.00 | 18.05 | C |

| | | | | | | | | | | | |
|---|---|---|---|---|---|---|---|---|---|---|---|
| ANISOU | 1452 | CB | TYR A | 578 | 2417 | 1988 | 2454 | −51 | −9 | 200 | C |

| | | | | | | | | | | |
|---|---|---|---|---|---|---|---|---|---|---|
| ATOM | 1453 | CG | TYR A | 578 | 28.616 | 37.965 | 80.797 | 1.00 | 17.53 | C |

| | | | | | | | | | | | |
|---|---|---|---|---|---|---|---|---|---|---|---|
| ANISOU | 1453 | CG | TYR A | 578 | 2369 | 2120 | 2173 | −157 | 100 | 80 | C |

| | | | | | | | | | | |
|---|---|---|---|---|---|---|---|---|---|---|
| ATOM | 1454 | CD1 | TYR A | 578 | 29.377 | 36.836 | 81.036 | 1.00 | 17.77 | C |

| | | | | | | | | | | | |
|---|---|---|---|---|---|---|---|---|---|---|---|
| ANISOU | 1454 | CD1 | TYR A | 578 | 2327 | 2085 | 2339 | −193 | 92 | 220 | C |

| | | | | | | | | | | |
|---|---|---|---|---|---|---|---|---|---|---|
| ATOM | 1455 | CD2 | TYR A | 578 | 28.955 | 39.125 | 81.509 | 1.00 | 17.90 | C |

| | | | | | | | | | | | |
|---|---|---|---|---|---|---|---|---|---|---|---|
| ANISOU | 1455 | CD2 | TYR A | 578 | 2503 | 2039 | 2258 | −265 | 23 | 192 | C |

| | | | | | | | | | | |
|---|---|---|---|---|---|---|---|---|---|---|
| ATOM | 1456 | CE1 | TYR A | 578 | 30.460 | 36.820 | 81.913 | 1.00 | 18.18 | C |

| | | | | | | | | | | | |
|---|---|---|---|---|---|---|---|---|---|---|---|
| ANISOU | 1456 | CE1 | TYR A | 578 | 2563 | 2145 | 2199 | −269 | −51 | 201 | C |

| | | | | | | | | | | |
|---|---|---|---|---|---|---|---|---|---|---|
| ATOM | 1457 | CE2 | TYR A | 578 | 30.000 | 39.140 | 82.409 | 1.00 | 18.63 | C |

| | | | | | | | | | | | |
|---|---|---|---|---|---|---|---|---|---|---|---|
| ANISOU | 1457 | CE2 | TYR A | 578 | 2506 | 2192 | 2381 | −171 | −48 | 211 | C |

| | | | | | | | | | | |
|---|---|---|---|---|---|---|---|---|---|---|
| ATOM | 1459 | CZ | TYR A | 578 | 30.767 | 38.002 | 82.588 | 1.00 | 19.75 | C |

| | | | | | | | | | | | |
|---|---|---|---|---|---|---|---|---|---|---|---|
| ANISOU | 1458 | CZ | TYR A | 578 | 2728 | 2199 | 2576 | −85 | −60 | 26 | C |

| | | | | | | | | | | |
|---|---|---|---|---|---|---|---|---|---|---|
| ATOM | 1459 | OH | TYR A | 578 | 31.839 | 38.006 | 83.459 | 1.00 | 20.94 | O |

| | | | | | | | | | | | |
|---|---|---|---|---|---|---|---|---|---|---|---|
| ANISOU | 1459 | OH | TYR A | 578 | 3103 | 2377 | 2475 | −225 | −188 | 383 | O |

| | | | | | | | | | | |
|---|---|---|---|---|---|---|---|---|---|---|
| ATOM | 1460 | N | ASP A | 579 | 26.364 | 35.218 | 81.553 | 1.00 | 16.24 | N |

| | | | | | | | | | | | |
|---|---|---|---|---|---|---|---|---|---|---|---|
| ANISOU | 1460 | N | ASP A | 579 | 2383 | 1562 | 2224 | −32 | −128 | 93 | N |

| | | | | | | | | | | |
|---|---|---|---|---|---|---|---|---|---|---|
| ATOM | 1461 | CA | ASP A | 579 | 26.597 | 33.756 | 81.601 | 1.00 | 15.40 | C |

| | | | | | | | | | | | |
|---|---|---|---|---|---|---|---|---|---|---|---|
| ANISOU | 1461 | CA | ASP A | 579 | 2375 | 1479 | 1997 | −149 | −153 | 31 | C |

| | | | | | | | | | | |
|---|---|---|---|---|---|---|---|---|---|---|
| ATOM | 1462 | C | ASP A | 579 | 25.363 | 32.895 | 81.521 | 1.00 | 15.59 | C |

| | | | | | | | | | | | |
|---|---|---|---|---|---|---|---|---|---|---|---|
| ANISOU | 1462 | C | ASP A | 579 | 2026 | 1757 | 2142 | −1 | 35 | 9 | C |

| | | | | | | | | | | |
|---|---|---|---|---|---|---|---|---|---|---|
| ATOM | 1463 | O | ASP A | 579 | 25.522 | 31.662 | 81.674 | 1.00 | 16.28 | O |

| | | | | | | | | | | | |
|---|---|---|---|---|---|---|---|---|---|---|---|
| ANISOU | 1463 | O | ASP A | 579 | 2433 | 1646 | 2108 | −37 | 199 | 9 | O |

| | | | | | | | | | | |
|---|---|---|---|---|---|---|---|---|---|---|
| ATOM | 1464 | CB | ASP A | 579 | 27.393 | 33.402 | 82.866 | 1.00 | 16.76 | C |

| | | | | | | | | | | | |
|---|---|---|---|---|---|---|---|---|---|---|---|
| ANISOU | 1464 | CB | ASP A | 579 | 2320 | 1856 | 2192 | −222 | −213 | 171 | C |

| | | | | | | | | | | |
|---|---|---|---|---|---|---|---|---|---|---|
| ATOM | 1465 | CG | ASP A | 579 | 28.892 | 33.467 | 82.657 | 1.00 | 18.53 | C |

| | | | | | | | | | | | |
|---|---|---|---|---|---|---|---|---|---|---|---|
| ANISOU | 1465 | CG | ASP A | 579 | 2559 | 2127 | 2355 | 55 | −69 | 56 | C |

| | | | | | | | | | | |
|---|---|---|---|---|---|---|---|---|---|---|
| ATOM | 1466 | OD1 | ASP A | 579 | 29.395 | 33.366 | 81.509 | 1.00 | 18.83 | O |

| | | | | | | | | | | | |
|---|---|---|---|---|---|---|---|---|---|---|---|
| ANISOU | 1466 | OD1 | ASP A | 579 | 2163 | 2493 | 2500 | −31 | −71 | 125 | O |

| | | | | | | | | | | |
|---|---|---|---|---|---|---|---|---|---|---|
| ATOM | 1467 | OD2 | ASP A | 579 | 29.610 | 33.626 | 83.673 | 1.00 | 20.44 | O |

| | | | | | | | | | | | |
|---|---|---|---|---|---|---|---|---|---|---|---|
| ANISOU | 1467 | OD2 | ASP A | 579 | 2765 | 2218 | 2782 | 59 | −321 | 171 | O |

| | | | | | | | | | | |
|---|---|---|---|---|---|---|---|---|---|---|
| ATOM | 1468 | N | CYS A | 580 | 24.182 | 33.463 | 81.341 | 1.00 | 14.58 | N |

| | | | | | | | | | | | |
|---|---|---|---|---|---|---|---|---|---|---|---|
| ANISOU | 1468 | N | CYS A | 580 | 1786 | 1749 | 2004 | −121 | −45 | 4 | N |

| | | | | | | | | | | |
|---|---|---|---|---|---|---|---|---|---|---|
| ATOM | 1469 | CA | CYS A | 580 | 22.970 | 32.593 | 81.394 | 1.00 | 14.75 | C |

| | | | | | | | | | | | |
|---|---|---|---|---|---|---|---|---|---|---|---|
| ANISOU | 1469 | CA | CYS A | 580 | 1887 | 1776 | 1941 | −146 | 73 | 49 | C |

| | | | | | | | | | | |
|---|---|---|---|---|---|---|---|---|---|---|
| ATOM | 1470 | C | CYS A | 580 | 23.109 | 31.389 | 80.475 | 1.00 | 15.30 | C |

| | | | | | | | | | | | |
|---|---|---|---|---|---|---|---|---|---|---|---|
| ANISOU | 1470 | C | CYS A | 580 | 2005 | 1882 | 1927 | −119 | −34 | 50 | C |

| | | | | | | | | | | |
|---|---|---|---|---|---|---|---|---|---|---|
| ATOM | 1471 | O | CYS A | 580 | 22.645 | 30.293 | 80.890 | 1.00 | 15.92 | O |

| | | | | | | | | | | | |
|---|---|---|---|---|---|---|---|---|---|---|---|
| ANISOU | 1471 | O | CYS A | 580 | 2281 | 1646 | 2122 | −91 | 156 | −60 | O |

| | | | | | | | | | | |
|---|---|---|---|---|---|---|---|---|---|---|
| ATOM | 1472 | CB | CYS A | 580 | 21.662 | 33.321 | 81.112 | 1.00 | 16.92 | C |

| | | | | | | | | | | | |
|---|---|---|---|---|---|---|---|---|---|---|---|
| ANISOU | 1472 | CB | CYS A | 580 | 2430 | 2088 | 1910 | 101 | 33 | 49 | C |

| | | | | | | | | | | |
|---|---|---|---|---|---|---|---|---|---|---|
| ATOM | 1473 | SG | CYS A | 580 | 21.746 | 34.182 | 79.490 | 1.00 | 15.01 | S |

| | | | | | | | | | | | |
|---|---|---|---|---|---|---|---|---|---|---|---|
| ANISOU | 1473 | SG | CYS A | 580 | 2141 | 1455 | 2108 | 50 | 101 | 14 | S |

| | | | | | | | | | | |
|---|---|---|---|---|---|---|---|---|---|---|
| ATOM | 1474 | N | GLY A | 581 | 23.559 | 31.538 | 79.246 | 1.00 | 15.26 | N |

| | | | | | | | | | | | |
|---|---|---|---|---|---|---|---|---|---|---|---|
| ANISOU | 1474 | N | GLY A | 581 | 2113 | 1666 | 2021 | 44 | 147 | 83 | N |

| | | | | | | | | | | |
|---|---|---|---|---|---|---|---|---|---|---|
| ATOM | 1475 | CA | GLY A | 581 | 23.611 | 30.365 | 78.313 | 1.00 | 15.15 | C |

| | | | | | | | | | | | |
|---|---|---|---|---|---|---|---|---|---|---|---|
| ANISOU | 1475 | CA | GLY A | 581 | 2305 | 1638 | 1815 | −29 | −224 | −2 | C |

| | | | | | | | | | | |
|---|---|---|---|---|---|---|---|---|---|---|
| ATOM | 1476 | C | GLY A | 581 | 24.626 | 29.341 | 78.879 | 1.00 | 12.80 | C |

| | | | | | | | | | | | |
|---|---|---|---|---|---|---|---|---|---|---|---|
| ANISOU | 1476 | C | GLY A | 581 | 1518 | 1669 | 1677 | −133 | 135 | 98 | C |

| | | | | | | | | | | |
|---|---|---|---|---|---|---|---|---|---|---|
| ATOM | 1477 | O | GLY A | 581 | 24.369 | 28.115 | 78.689 | 1.00 | 16.14 | O |

| | | | | | | | | | | | |
|---|---|---|---|---|---|---|---|---|---|---|---|
| ANISOU | 1477 | O | GLY A | 581 | 2411 | 1526 | 2194 | 74 | 128 | 65 | O |

| | | | | | | | | | | |
|---|---|---|---|---|---|---|---|---|---|---|
| ATOM | 1478 | N | ILE A | 582 | 25.694 | 29.752 | 79.489 | 1.00 | 13.89 | N |

| | | | | | | | | | | | |
|---|---|---|---|---|---|---|---|---|---|---|---|
| ANISOU | 1478 | N | ILE A | 582 | 2309 | 1315 | 1655 | 145 | 100 | 105 | N |

| | | | | | | | | | | |
|---|---|---|---|---|---|---|---|---|---|---|
| ATOM | 1479 | CA | ILE A | 582 | 26.664 | 28.816 | 80.097 | 1.00 | 11.67 | C |

| | | | | | | | | | | | |
|---|---|---|---|---|---|---|---|---|---|---|---|
| ANISOU | 1479 | CA | ILE A | 582 | 942 | 1769 | 1723 | 2 | 25 | 110 | C |

| | | | | | | | | | | |
|---|---|---|---|---|---|---|---|---|---|---|
| ATOM | 1480 | C | ILE A | 582 | 25.980 | 28.097 | 81.274 | 1.00 | 14.76 | C |

| | | | | | | | | | | | |
|---|---|---|---|---|---|---|---|---|---|---|---|
| ANISOU | 1480 | C | ILE A | 582 | 2254 | 1734 | 1621 | 235 | −19 | 111 | C |

| | | | | | | | | | | |
|---|---|---|---|---|---|---|---|---|---|---|
| ATOM | 1481 | O | ILE A | 582 | 26.205 | 26.886 | 81.440 | 1.00 | 16.85 | O |

| | | | | | | | | | | | |
|---|---|---|---|---|---|---|---|---|---|---|---|
| ANISOU | 1481 | O | ILE A | 582 | 2551 | 1737 | 2117 | −13 | −5 | 89 | O |

| | | | | | | | | | | |
|---|---|---|---|---|---|---|---|---|---|---|
| ATOM | 1482 | CB | ILE A | 582 | 27.966 | 29.614 | 80.495 | 1.00 | 14.03 | C |

| | | | | | | | | | | | |
|---|---|---|---|---|---|---|---|---|---|---|---|
| ANISOU | 1482 | CB | ILE A | 582 | 2097 | 1580 | 1655 | −29 | −270 | 186 | C |

| | | | | | | | | | | |
|---|---|---|---|---|---|---|---|---|---|---|
| ATOM | 1483 | CG1 | ILE A | 582 | 28.659 | 29.974 | 79.220 | 1.00 | 15.12 | C |

| | | | | | | | | | | | |
|---|---|---|---|---|---|---|---|---|---|---|---|
| ANISOU | 1483 | CG1 | ILE A | 582 | 1710 | 1814 | 2222 | 98 | 181 | −32 | C |

| | | | | | | | | | | |
|---|---|---|---|---|---|---|---|---|---|---|
| ATOM | 1484 | CG2 | ILE A | 582 | 28.785 | 28.740 | 81.423 | 1.00 | 14.20 | C |

| | | | | | | | | | | | |
|---|---|---|---|---|---|---|---|---|---|---|---|
| ANISOU | 1484 | CG2 | ILE A | 582 | 1517 | 1840 | 1938 | −94 | −207 | 270 | C |

| | | | | | | | | | | |
|---|---|---|---|---|---|---|---|---|---|---|
| ATOM | 1485 | CD1 | ILE A | 582 | 29.291 | 28.852 | 78.392 | 1.00 | 17.25 | C |

| | | | | | | | | | | | |
|---|---|---|---|---|---|---|---|---|---|---|---|
| ANISOU | 1485 | CD1 | ILE A | 582 | 2584 | 2012 | 1978 | −73 | 221 | −182 | C |

| | | | | | | | | | | |
|---|---|---|---|---|---|---|---|---|---|---|
| ATOM | 1486 | N | TYR A | 583 | 25.198 | 28.807 | 82.100 | 1.00 | 14.54 | N |

| | | | | | | | | | | | |
|---|---|---|---|---|---|---|---|---|---|---|---|
| ANISOU | 1486 | N | TYR A | 583 | 1670 | 1839 | 2016 | −52 | 192 | 33 | N |

| | | | | | | | | | | |
|---|---|---|---|---|---|---|---|---|---|---|
| ATOM | 1487 | CA | TYR A | 583 | 24.513 | 28.141 | 83.214 | 1.00 | 15.88 | C |

| | | | | | | | | | | | |
|---|---|---|---|---|---|---|---|---|---|---|---|
| ANISOU | 1487 | CA | TYR A | 583 | 2282 | 1888 | 1886 | 99 | 44 | 89 | C |

| | | | | | | | | | | |
|---|---|---|---|---|---|---|---|---|---|---|
| ATOM | 1488 | C | TYR A | 583 | 23.505 | 27.151 | 82.647 | 1.00 | 14.88 | C |

| | | | | | | | | | | | |
|---|---|---|---|---|---|---|---|---|---|---|---|
| ANISOU | 1488 | C | TYR A | 583 | 1675 | 1970 | 2011 | −51 | −43 | 77 | C |

| | | | | | | | | | | |
|---|---|---|---|---|---|---|---|---|---|---|
| ATOM | 1489 | O | TYR A | 583 | 23.331 | 28.047 | 83.234 | 1.00 | 18.28 | O |

| | | | | | | | | | | | |
|---|---|---|---|---|---|---|---|---|---|---|---|
| ANISOU | 1489 | O | TYR A | 583 | 2221 | 1849 | 2117 | 32 | 106 | 9 | O |

| | | | | | | | | | | |
|---|---|---|---|---|---|---|---|---|---|---|
| ATOM | 1490 | CB | TYR A | 583 | 23.892 | 29.165 | 84.154 | 1.00 | 15.73 | C |

| | | | | | | | | | | | |
|---|---|---|---|---|---|---|---|---|---|---|---|
| ANISOU | 1490 | CB | TYR A | 583 | 2254 | 1807 | 1918 | 0 | −54 | −20 | C |

| | | | | | | | | | | |
|---|---|---|---|---|---|---|---|---|---|---|
| ATOM | 1491 | CG | TYR A | 583 | 24.884 | 29.693 | 85.160 | 1.00 | 18.77 | C |

| | | | | | | | | | | | |
|---|---|---|---|---|---|---|---|---|---|---|---|
| ANISOU | 1491 | CG | TYR A | 583 | 1988 | 2161 | 2224 | −35 | −82 | 32 | C |

| | | | | | | | | | | |
|---|---|---|---|---|---|---|---|---|---|---|
| ATOM | 1492 | CD1 | TYR A | 583 | 25.956 | 30.477 | 84.772 | 1.00 | 16.09 | C |

| | | | | | | | | | | | |
|---|---|---|---|---|---|---|---|---|---|---|---|
| ANISOU | 1492 | CD1 | TYR A | 583 | 2224 | 1720 | 2170 | 14 | 13 | −41 | C |

| | | | | | | | | | | |
|---|---|---|---|---|---|---|---|---|---|---|
| ATOM | 1493 | CD2 | TYR A | 583 | 24.758 | 29.358 | 86.520 | 1.00 | 17.99 | C |

| | | | | | | | | | | | |
|---|---|---|---|---|---|---|---|---|---|---|---|
| ANISOU | 1493 | CD2 | TYR A | 583 | 2333 | 2237 | 2264 | −19 | 21 | 83 | C |

| | | | | | | | | | | |
|---|---|---|---|---|---|---|---|---|---|---|
| ATOM | 1494 | CE1 | TYR A | 583 | 26.870 | 30.952 | 85.725 | 1.00 | 18.44 | C |

| | | | | | | | | | | | |
|---|---|---|---|---|---|---|---|---|---|---|---|
| ANISOU | 1494 | CE1 | TYR A | 583 | 2273 | 1814 | 2159 | 0 | −103 | 65 | C |

| | | | | | | | | | | |
|---|---|---|---|---|---|---|---|---|---|---|
| ATOM | 1495 | CE2 | TYR A | 583 | 25.676 | 29.799 | 87.454 | 1.00 | 19.12 | C |

| | | | | | | | | | | | |
|---|---|---|---|---|---|---|---|---|---|---|---|
| ANISOU | 1495 | CE2 | TYR A | 583 | 2700 | 2169 | 2399 | −77 | −3 | 48 | C |

| | | | | | | | | | | |
|---|---|---|---|---|---|---|---|---|---|---|
| ATOM | 1496 | CZ | TYR A | 583 | 26.715 | 30.622 | 87.051 | 1.00 | 18.47 | C |

| | | | | | | | | | | | |
|---|---|---|---|---|---|---|---|---|---|---|---|
| ANISOU | 1496 | CZ | TYR A | 583 | 2359 | 2362 | 2296 | −46 | 19 | 34 | C |

| | | | | | | | | | | |
|---|---|---|---|---|---|---|---|---|---|---|
| ATOM | 1497 | OH | TYR A | 583 | 27.676 | 31.089 | 87.931 | 1.00 | 19.08 | O |

| | | | | | | | | | | | |
|---|---|---|---|---|---|---|---|---|---|---|---|
| ANISOU | 1497 | OH | TYR A | 583 | 2802 | 2068 | 2378 | −85 | −99 | −10 | O |

| | | | | | | | | | | |
|---|---|---|---|---|---|---|---|---|---|---|
| ATOM | 1498 | N | VAL A | 584 | 22.856 | 27.430 | 81.524 | 1.00 | 14.35 | N |

| | | | | | | | | | | | |
|---|---|---|---|---|---|---|---|---|---|---|---|
| ANISOU | 1498 | N | VAL A | 584 | 2297 | 1448 | 1706 | −6 | 40 | 26 | N |

| | | | | | | | | | | |
|---|---|---|---|---|---|---|---|---|---|---|
| ATOM | 1499 | CA | VAL A | 584 | 21.917 | 26.471 | 80.898 | 1.00 | 13.49 | C |

| | | | | | | | | | | | |
|---|---|---|---|---|---|---|---|---|---|---|---|
| ANISOU | 1499 | CA | VAL A | 584 | 1239 | 1687 | 2199 | −25 | −198 | −53 | C |

| | | | | | | | | | | |
|---|---|---|---|---|---|---|---|---|---|---|
| ATOM | 1500 | C | VAL A | 584 | 22.739 | 25.200 | 80.575 | 1.00 | 15.85 | C |

| | | | | | | | | | | | |
|---|---|---|---|---|---|---|---|---|---|---|---|
| ANISOU | 1500 | C | VAL A | 584 | 2076 | 1654 | 2293 | −75 | 84 | −156 | C |

| | | | | | | | | | | |
|---|---|---|---|---|---|---|---|---|---|---|
| ATOM | 1501 | O | VAL A | 584 | 22.297 | 24.087 | 80.838 | 1.00 | 18.30 | O |

| | | | | | | | | | | | |
|---|---|---|---|---|---|---|---|---|---|---|---|
| ANISOU | 1501 | O | VAL A | 584 | 2203 | 1711 | 2280 | 38 | 89 | 137 | O |

| | | | | | | | | | | |
|---|---|---|---|---|---|---|---|---|---|---|
| ATOM | 1502 | CB | VAL A | 584 | 21.225 | 27.070 | 79.676 | 1.00 | 14.84 | C |

| | | | | | | | | | | | |
|---|---|---|---|---|---|---|---|---|---|---|---|
| ANISOU | 1502 | CB | VAL A | 584 | 2065 | 1802 | 1898 | 148 | 93 | −168 | C |

| | | | | | | | | | | |
|---|---|---|---|---|---|---|---|---|---|---|
| ATOM | 1503 | CG1 | VAL A | 584 | 20.504 | 25.998 | 78.788 | 1.00 | 16.28 | C |

| | | | | | | | | | | | |
|---|---|---|---|---|---|---|---|---|---|---|---|
| ANISOU | 1503 | CG1 | VAL A | 584 | 2014 | 1854 | 2317 | −183 | −253 | −128 | C |

| | | | | | | | | | | |
|---|---|---|---|---|---|---|---|---|---|---|
| ATOM | 1504 | CG2 | VAL A | 584 | 20.116 | 28.036 | 80.136 | 1.00 | 13.73 | C |

| | | | | | | | | | | | |
|---|---|---|---|---|---|---|---|---|---|---|---|
| ANISOU | 1504 | CG2 | VAL A | 584 | 1848 | 1723 | 1848 | 128 | 74 | 24 | C |

| | | | | | | | | | | |
|---|---|---|---|---|---|---|---|---|---|---|
| ATOM | 1505 | N | CYS A | 585 | 23.890 | 25.410 | 79.928 | 1.00 | 14.55 | N |

| | | | | | | | | | | | |
|---|---|---|---|---|---|---|---|---|---|---|---|
| ANISOU | 1505 | N | CYS A | 585 | 1829 | 1839 | 1861 | 149 | 84 | −212 | N |

| | | | | | | | | | | |
|---|---|---|---|---|---|---|---|---|---|---|
| ATOM | 1506 | CA | CYS A | 585 | 24.745 | 24.225 | 79.586 | 1.00 | 14.62 | C |

| | | | | | | | | | | | |
|---|---|---|---|---|---|---|---|---|---|---|---|
| ANISOU | 1506 | CA | CYS A | 585 | 1925 | 1546 | 2085 | 63 | 68 | −103 | C |

| | | | | | | | | | | |
|---|---|---|---|---|---|---|---|---|---|---|
| ATOM | 1507 | C | CYS A | 585 | 25.198 | 23.483 | 80.821 | 1.00 | 15.99 | C |

| | | | | | | | | | | | |
|---|---|---|---|---|---|---|---|---|---|---|---|
| ANISOU | 1507 | C | CYS A | 585 | 2138 | 1672 | 2286 | 188 | 93 | 24 | C |

| | | | | | | | | | | |
|---|---|---|---|---|---|---|---|---|---|---|
| ATOM | 1508 | O | CYS A | 585 | 25.133 | 22.222 | 80.860 | 1.00 | 15.88 | O |

| | | | | | | | | | | | |
|---|---|---|---|---|---|---|---|---|---|---|---|
| ANISOU | 1508 | O | CYS A | 585 | 2047 | 1805 | 2374 | −109 | 216 | −105 | O |

| | | | | | | | | | | |
|---|---|---|---|---|---|---|---|---|---|---|
| ATOM | 1509 | CB | CYS A | 585 | 25.953 | 24.692 | 78.743 | 1.00 | 16.25 | C |

| | | | | | | | | | | | |
|---|---|---|---|---|---|---|---|---|---|---|---|
| ANISOU | 1509 | CB | CYS A | 585 | 2385 | 1769 | 2019 | −125 | 137 | 117 | C |

| | | | | | | | | | | |
|---|---|---|---|---|---|---|---|---|---|---|
| ATOM | 1510 | SG | CYS A | 585 | 25.410 | 25.282 | 77.100 | 1.00 | 15.94 | S |

| | | | | | | | | | | | |
|---|---|---|---|---|---|---|---|---|---|---|---|
| ANISOU | 1510 | SG | CYS A | 585 | 2463 | 1609 | 1983 | −35 | 232 | 134 | S |

| | | | | | | | | | | |
|---|---|---|---|---|---|---|---|---|---|---|
| ATOM | 1511 | N | MET A | 586 | 25.607 | 24.149 | 81.898 | 1.00 | 16.65 | N |

| | | | | | | | | | | | |
|---|---|---|---|---|---|---|---|---|---|---|---|
| ANISOU | 1511 | N | MET A | 586 | 2185 | 1896 | 2246 | −11 | −3 | 75 | N |

| | | | | | | | | | | |
|---|---|---|---|---|---|---|---|---|---|---|
| ATOM | 1512 | CA | MET A | 586 | 26.014 | 23.444 | 83.132 | 1.00 | 18.64 | C |

| | | | | | | | | | | | |
|---|---|---|---|---|---|---|---|---|---|---|---|
| ANISOU | 1512 | CA | MET A | 588 | 2076 | 1907 | 2338 | 20 | −31 | 109 | C |

| | | | | | | | | | | |
|---|---|---|---|---|---|---|---|---|---|---|
| ATOM | 1513 | C | MET A | 586 | 24.812 | 22.720 | 83.714 | 1.00 | 18.83 | C |

| | | | | | | | | | | | |
|---|---|---|---|---|---|---|---|---|---|---|---|
| ANISOU | 1513 | C | MET A | 586 | 2199 | 1798 | 2397 | 59 | 87 | 79 | C |

| | | | | | | | | | | |
|---|---|---|---|---|---|---|---|---|---|---|
| ATOM | 1514 | O | MET A | 586 | 24.916 | 21.580 | 84.199 | 1.00 | 18.82 | O |

| | | | | | | | | | | | |
|---|---|---|---|---|---|---|---|---|---|---|---|
| ANISOU | 1514 | O | MET A | 586 | 2377 | 1660 | 2353 | 80 | 111 | 80 | O |

| | | | | | | | | | | |
|---|---|---|---|---|---|---|---|---|---|---|
| ATOM | 1515 | CB | MET A | 586 | 26.665 | 24.349 | 84.176 | 1.00 | 17.32 | C |

| | | | | | | | | | | | |
|---|---|---|---|---|---|---|---|---|---|---|---|
| ANISOU | 1515 | CB | MET A | 586 | 2143 | 2171 | 2266 | −74 | −39 | 84 | C |

| | | | | | | | | | | |
|---|---|---|---|---|---|---|---|---|---|---|
| ATOM | 1516 | CG | MET A | 586 | 27.984 | 24.955 | 83.586 | 1.00 | 17.11 | C |

| | | | | | | | | | | | |
|---|---|---|---|---|---|---|---|---|---|---|---|
| ANISOU | 1516 | CG | MET A | 586 | 2322 | 2029 | 2150 | −153 | 160 | −64 | C |

| | | | | | | | | | | |
|---|---|---|---|---|---|---|---|---|---|---|
| ATOM | 1517 | SD | MET A | 586 | 28.929 | 25.753 | 84.903 | 1.00 | 17.40 | S |

| | | | | | | | | | | | |
|---|---|---|---|---|---|---|---|---|---|---|---|
| ANISOU | 1517 | SD | MET A | 586 | 2281 | 1987 | 2344 | −108 | −37 | 71 | S |

| | | | | | | | | | | |
|---|---|---|---|---|---|---|---|---|---|---|
| ATOM | 1518 | CE | MET A | 586 | 27.885 | 27.197 | 85.107 | 1.00 | 18.54 | C |

| | | | | | | | | | | | |
|---|---|---|---|---|---|---|---|---|---|---|---|
| ANISOU | 1518 | CE | MET A | 586 | 2749 | 1903 | 2393 | 33 | −59 | 113 | C |

| | | | | | | | | | | |
|---|---|---|---|---|---|---|---|---|---|---|
| ATOM | 1519 | N | ASN A | 587 | 23.624 | 23.345 | 83.747 | 1.00 | 14.87 | N |

| | | | | | | | | | | | |
|---|---|---|---|---|---|---|---|---|---|---|---|
| ANISOU | 1519 | N | ASN A | 587 | 1994 | 1530 | 2125 | −56 | 81 | −73 | N |

| | | | | | | | | | | |
|---|---|---|---|---|---|---|---|---|---|---|
| ATOM | 1520 | CA | ASN A | 587 | 22.424 | 22.671 | 84.251 | 1.00 | 16.26 | C |

| | | | | | | | | | | | |
|---|---|---|---|---|---|---|---|---|---|---|---|
| ANISOU | 1520 | CA | ASN A | 587 | 1976 | 1864 | 2337 | 61 | 169 | 49 | C |

| | | | | | | | | | | |
|---|---|---|---|---|---|---|---|---|---|---|
| ATOM | 1521 | C | ASN A | 587 | 22.050 | 21.459 | 83.429 | 1.00 | 16.93 | C |

| | | | | | | | | | | | |
|---|---|---|---|---|---|---|---|---|---|---|---|
| ANISOU | 1521 | C | ASN A | 587 | 2086 | 1969 | 2380 | −52 | −21 | 53 | C |

| | | | | | | | | | | |
|---|---|---|---|---|---|---|---|---|---|---|
| ATOM | 1522 | O | ASN A | 587 | 21.545 | 20.458 | 84.021 | 1.00 | 19.17 | O |

| | | | | | | | | | | | |
|---|---|---|---|---|---|---|---|---|---|---|---|
| ANISOU | 1522 | O | ASN A | 587 | 2601 | 1963 | 2720 | −43 | 137 | 198 | O |

| | | | | | | | | | | |
|---|---|---|---|---|---|---|---|---|---|---|
| ATOM | 1523 | CB | ASN A | 587 | 21.243 | 23.661 | 84.322 | 1.00 | 16.58 | C |

| | | | | | | | | | | | |
|---|---|---|---|---|---|---|---|---|---|---|---|
| ANISOU | 1523 | CB | ASN A | 587 | 2216 | 1901 | 2184 | 196 | 62 | 55 | C |

| | | | | | | | | | | |
|---|---|---|---|---|---|---|---|---|---|---|
| ATOM | 1524 | CG | ASN A | 587 | 21.249 | 24.420 | 85.628 | 1.00 | 17.93 | C |

| | | | | | | | | | | | |
|---|---|---|---|---|---|---|---|---|---|---|---|
| ANISOU | 1524 | CG | ASN A | 587 | 2440 | 2145 | 2227 | 135 | 83 | 81 | C |

| | | | | | | | | | | |
|---|---|---|---|---|---|---|---|---|---|---|
| ATOM | 1525 | OD1 | ASN A | 587 | 21.423 | 23.843 | 86.692 | 1.00 | 18.16 | O |

| | | | | | | | | | | | |
|---|---|---|---|---|---|---|---|---|---|---|---|
| ANISOU | 1525 | OD1 | ASN A | 587 | 2542 | 2048 | 2309 | 197 | 181 | 210 | O |

| | | | | | | | | | | |
|---|---|---|---|---|---|---|---|---|---|---|
| ATOM | 1526 | ND2 | ASN A | 587 | 20.952 | 25.724 | 85.554 | 1.00 | 17.76 | N |

| | | | | | | | | | | | |
|---|---|---|---|---|---|---|---|---|---|---|---|
| ANISOU | 1526 | ND2 | ASN A | 587 | 2369 | 2019 | 2361 | 122 | 345 | 2 | N |

| | | | | | | | | | | |
|---|---|---|---|---|---|---|---|---|---|---|
| ATOM | 1527 | N | THR A | 588 | 22.316 | 21.472 | 82.133 | 1.00 | 17.13 | N |

| | | | | | | | | | | | |
|---|---|---|---|---|---|---|---|---|---|---|---|
| ANISOU | 1527 | N | THR A | 588 | 2480 | 1643 | 2386 | 9 | 28 | 9 | N |

| | | | | | | | | | | |
|---|---|---|---|---|---|---|---|---|---|---|
| ATOM | 1528 | CA | THR A | 588 | 22.011 | 20.287 | 81.285 | 1.00 | 16.51 | C |

| | | | | | | | | | | | |
|---|---|---|---|---|---|---|---|---|---|---|---|
| ANISOU | 1528 | CA | THR A | 588 | 2153 | 1685 | 2436 | −35 | 35 | 30 | C |

| | | | | | | | | | | |
|---|---|---|---|---|---|---|---|---|---|---|
| ATOM | 1529 | C | THR A | 588 | 23.054 | 19.219 | 81.598 | 1.00 | 17.11 | C |

| | | | | | | | | | | | |
|---|---|---|---|---|---|---|---|---|---|---|---|
| ANISOU | 1529 | C | THR A | 588 | 2223 | 1914 | 2365 | −6 | −188 | 54 | C |

| | | | | | | | | | | |
|---|---|---|---|---|---|---|---|---|---|---|
| ATOM | 1530 | O | THR A | 588 | 22.697 | 18.035 | 81.711 | 1.00 | 18.81 | O |

| | | | | | | | | | | | |
|---|---|---|---|---|---|---|---|---|---|---|---|
| ANISOU | 1530 | O | THR A | 588 | 2513 | 1878 | 2755 | −142 | 75 | 40 | O |

| | | | | | | | | | |
|---|---|---|---|---|---|---|---|---|---|
| ATOM | 1531 | CB | THR A | 588 | 22.026 | 20.673 | 79.818 | 1.00 | 17.47 C |

| | | | | | | | | | | | |
|---|---|---|---|---|---|---|---|---|---|---|---|
| ANISOU | 1531 | CB | THR A | 588 | 2187 | 2155 | 2297 | 172 | −2 | −123 | C |

| | | | | | | | | | | |
|---|---|---|---|---|---|---|---|---|---|---|
| ATOM | 1532 | OG1 | THR A | 588 | 21.090 | 21.795 | 79.642 | 1.00 | 16.25 | O |

| | | | | | | | | | | | |
|---|---|---|---|---|---|---|---|---|---|---|---|
| ANISOU | 1532 | OG1 | THR A | 588 | 2279 | 1995 | 1900 | 185 | 19 | 146 | O |

| | | | | | | | | | | |
|---|---|---|---|---|---|---|---|---|---|---|
| ATOM | 1533 | CG2 | THR A | 588 | 21.626 | 19.570 | 78.851 | 1.00 | 18.62 | C |

| | | | | | | | | | | | |
|---|---|---|---|---|---|---|---|---|---|---|---|
| ANISOU | 1533 | CG2 | THR A | 588 | 2485 | 2105 | 2483 | 206 | −10 | −123 | C |

| | | | | | | | | | | |
|---|---|---|---|---|---|---|---|---|---|---|
| ATOM | 1534 | N | LEU A | 589 | 24.324 | 19.627 | 81.659 | 1.00 | 17.02 | N |

| | | | | | | | | | | | |
|---|---|---|---|---|---|---|---|---|---|---|---|
| ANISOU | 1534 | N | LEU A | 589 | 2103 | 1940 | 2424 | 138 | 30 | 66 | N |

| | | | | | | | | | | |
|---|---|---|---|---|---|---|---|---|---|---|
| ATOM | 1535 | CA | LEU A | 589 | 25.396 | 18.656 | 81.965 | 1.00 | 16.26 | C |

| | | | | | | | | | | | |
|---|---|---|---|---|---|---|---|---|---|---|---|
| ANISOU | 1535 | CA | LEU A | 589 | 2115 | 1733 | 2330 | 29 | −46 | 29 | C |

| | | | | | | | | | | |
|---|---|---|---|---|---|---|---|---|---|---|
| ATOM | 1536 | C | LEU A | 589 | 25.164 | 17.998 | 83.308 | 1.00 | 17.93 | C |

| | | | | | | | | | | | |
|---|---|---|---|---|---|---|---|---|---|---|---|
| ANISOU | 1536 | C | LEU A | 589 | 2438 | 2010 | 2363 | 16 | −91 | 52 | C |

| | | | | | | | | | | |
|---|---|---|---|---|---|---|---|---|---|---|
| ATOM | 1537 | O | LEU A | 589 | 25.126 | 16.737 | 83.381 | 1.00 | 19.00 | O |

| | | | | | | | | | | | |
|---|---|---|---|---|---|---|---|---|---|---|---|
| ANISOU | 1537 | O | LEU A | 589 | 2729 | 1916 | 2573 | 93 | −51 | 47 | O |

| | | | | | | | | | | |
|---|---|---|---|---|---|---|---|---|---|---|
| ATOM | 1538 | CB | LEU A | 589 | 26.732 | 19.403 | 81.963 | 1.00 | 17.41 | C |

| | | | | | | | | | | | |
|---|---|---|---|---|---|---|---|---|---|---|---|
| ANISOU | 1538 | CB | LEU A | 589 | 2107 | 2050 | 2457 | 36 | −46 | 3 | C |

| | | | | | | | | | | |
|---|---|---|---|---|---|---|---|---|---|---|
| ATOM | 1539 | CG | LEU A | 589 | 27.962 | 18.670 | 82.523 | 1.00 | 17.90 | C |

| | | | | | | | | | | | |
|---|---|---|---|---|---|---|---|---|---|---|---|
| ANISOU | 1539 | CG | LEU A | 589 | 2019 | 2230 | 2553 | 108 | 64 | −41 | C |

| | | | | | | | | | | |
|---|---|---|---|---|---|---|---|---|---|---|
| ATOM | 1540 | CD1 | LEU A | 589 | 28.251 | 17.540 | 81.558 | 1.00 | 20.93 | C |

| | | | | | | | | | | | |
|---|---|---|---|---|---|---|---|---|---|---|---|
| ANISOU | 1540 | CD1 | LEU A | 589 | 2940 | 2349 | 2665 | 92 | −74 | −147 | C |

| | | | | | | | | | | |
|---|---|---|---|---|---|---|---|---|---|---|
| ATOM | 1541 | CD2 | LEU A | 589 | 29.115 | 19.669 | 82.572 | 1.00 | 19.00 | C |

| | | | | | | | | | | | |
|---|---|---|---|---|---|---|---|---|---|---|---|
| ANISOU | 1541 | CD2 | LEU A | 589 | 2119 | 2499 | 2600 | −83 | 24 | 4 | C |

| | | | | | | | | | | |
|---|---|---|---|---|---|---|---|---|---|---|
| ATOM | 1542 | N | TYR A | 590 | 24.943 | 18.746 | 84.356 | 1.00 | 17.57 | N |

| | | | | | | | | | | | |
|---|---|---|---|---|---|---|---|---|---|---|---|
| ANISOU | 1542 | N | TYR A | 590 | 2164 | 2120 | 2393 | −8 | −66 | −6 | N |

| | | | | | | | | | | |
|---|---|---|---|---|---|---|---|---|---|---|
| ATOM | 1543 | CA | TYR A | 590 | 24.757 | 18.186 | 85.696 | 1.00 | 19.35 | C |

| | | | | | | | | | | | |
|---|---|---|---|---|---|---|---|---|---|---|---|
| ANISOU | 1543 | CA | TYR A | 590 | 2457 | 2321 | 2573 | −163 | 88 | 69 | C |

| | | | | | | | | | | |
|---|---|---|---|---|---|---|---|---|---|---|
| ATOM | 1544 | C | TYR A | 590 | 23.427 | 17.447 | 85.756 | 1.00 | 19.84 | C |

| | | | | | | | | | | | |
|---|---|---|---|---|---|---|---|---|---|---|---|
| ANISOU | 1544 | C | TYR A | 590 | 2584 | 2248 | 2707 | −36 | −78 | 68 | C |

| | | | | | | | | | | |
|---|---|---|---|---|---|---|---|---|---|---|
| ATOM | 1545 | O | TYR A | 590 | 23.429 | 16.355 | 86.324 | 1.00 | 19.99 | O |

| | | | | | | | | | | | |
|---|---|---|---|---|---|---|---|---|---|---|---|
| ANISOU | 1545 | O | TYR A | 590 | 2457 | 2270 | 2868 | 76 | −19 | 254 | O |

| | | | | | | | | | | |
|---|---|---|---|---|---|---|---|---|---|---|
| ATOM | 1546 | CB | TYR A | 590 | 24.808 | 19.275 | 86.791 | 1.00 | 18.4 | C |

| | | | | | | | | | | |
|---|---|---|---|---|---|---|---|---|---|---|
| ANISOU | 1546 | CB | TYR A | 590 | 2249 | 2297 | 2472 | −75 | 74 | 29 C |

| | | | | | | | | | | |
|---|---|---|---|---|---|---|---|---|---|---|
| ATOM | 1547 | CG | TYR A | 590 | 26.243 | 19.696 | 97.110 | 1.00 | 19.70 | C |

| | | | | | | | | | | | |
|---|---|---|---|---|---|---|---|---|---|---|---|
| ANISOU | 1547 | CG | TYR A | 590 | 2405 | 2494 | 2588 | −166 | 9 | 41 | C |

| | | | | | | | | | | |
|---|---|---|---|---|---|---|---|---|---|---|
| ATOM | 1548 | CD1 | TYR A | 590 | 27.110 | 20.315 | 86.197 | 1.00 | 18.32 | C |

| | | | | | | | | | | | |
|---|---|---|---|---|---|---|---|---|---|---|---|
| ANISOU | 1548 | CD1 | TYR A | 590 | 2317 | 2126 | 2519 | −22 | −137 | 177 | C |

| | | | | | | | | | | |
|---|---|---|---|---|---|---|---|---|---|---|
| ATOM | 1549 | CD2 | TYR A | 590 | 26.736 | 19.417 | 88.377 | 1.00 | 20.50 | C |

| | | | | | | | | | | | |
|---|---|---|---|---|---|---|---|---|---|---|---|
| ANISOU | 1549 | CD2 | TYR A | 590 | 2611 | 2448 | 2730 | −165 | −113 | 179 | C |

| | | | | | | | | | | |
|---|---|---|---|---|---|---|---|---|---|---|
| ATOM | 1550 | CE1 | TYR A | 590 | 28.396 | 20.662 | 86.579 | 1.00 | 20.91 | C |

| | | | | | | | | | | | |
|---|---|---|---|---|---|---|---|---|---|---|---|
| ANISOU | 1550 | CE1 | TYR A | 590 | 2746 | 2379 | 2820 | 3 | −108 | 197 | C |

| | | | | | | | | | | |
|---|---|---|---|---|---|---|---|---|---|---|
| ATOM | 1551 | CE2 | TYR A | 590 | 28.015 | 19.757 | 88.767 | 1.00 | 22.19 | C |

| | | | | | | | | | | | |
|---|---|---|---|---|---|---|---|---|---|---|---|
| ANISOU | 1551 | CE2 | TYR A | 590 | 2759 | 2738 | 2932 | −119 | −154 | 162 | C |

| | | | | | | | | | | |
|---|---|---|---|---|---|---|---|---|---|---|
| ATOM | 1552 | CZ | TYR A | 590 | 28.845 | 20.365 | 87.839 | 1.00 | 21.25 | C |

| | | | | | | | | | | | |
|---|---|---|---|---|---|---|---|---|---|---|---|
| ANISOU | 1552 | CZ | TYR A | 590 | 2464 | 2849 | 2759 | −119 | −129 | 18 | C |

| | | | | | | | | | | |
|---|---|---|---|---|---|---|---|---|---|---|
| ATOM | 1553 | OH | TYR A | 590 | 30.151 | 20.716 | 88.161 | 1.00 | 24.98 | O |

| | | | | | | | | | | | |
|---|---|---|---|---|---|---|---|---|---|---|---|
| ANISOU | 1553 | OH | TYR A | 590 | 2980 | 3270 | 3242 | −111 | −299 | 61 | O |

| | | | | | | | | | | |
|---|---|---|---|---|---|---|---|---|---|---|
| ATOM | 1554 | N | GLY A | 591 | 22.369 | 18.004 | 85.199 | 1.00 | 17.96 | N |

| | | | | | | | | | | | |
|---|---|---|---|---|---|---|---|---|---|---|---|
| ANISOU | 1554 | N | GLY A | 591 | 2267 | 2041 | 2515 | −97 | −167 | 82 | N |

| | | | | | | | | | | |
|---|---|---|---|---|---|---|---|---|---|---|
| ATOM | 1555 | CA | GLY A | 591 | 21.056 | 17.274 | 85.231 | 1.00 | 19.00 | C |

| | | | | | | | | | | | |
|---|---|---|---|---|---|---|---|---|---|---|---|
| ANISOU | 1555 | CA | GLY A | 591 | 2291 | 2242 | 2685 | −41 | −13 | 149 | C |

| | | | | | | | | | | |
|---|---|---|---|---|---|---|---|---|---|---|
| ATOM | 1556 | C | GLY A | 591 | 21.187 | 15.972 | 84.452 | 1.00 | 20.19 | C |

| | | | | | | | | | | | |
|---|---|---|---|---|---|---|---|---|---|---|---|
| ANISOU | 1556 | C | GLY A | 591 | 2656 | 2329 | 2687 | −117 | −83 | 125 | C |

| | | | | | | | | | | |
|---|---|---|---|---|---|---|---|---|---|---|
| ATOM | 1557 | O | GLY A | 591 | 20.603 | 14.978 | 84.956 | 1.00 | 21.39 | O |

| | | | | | | | | | | | |
|---|---|---|---|---|---|---|---|---|---|---|---|
| ANISOU | 1557 | O | GLY A | 591 | 2711 | 2305 | 3112 | −15 | −123 | 404 | O |

| | | | | | | | | | | |
|---|---|---|---|---|---|---|---|---|---|---|
| ATOM | 1558 | N | SER A | 592 | 21.855 | 15.896 | 83.318 | 1.00 | 19.55 | N |

| | | | | | | | | | | | |
|---|---|---|---|---|---|---|---|---|---|---|---|
| ANISOU | 1558 | N | SER A | 592 | 2498 | 2220 | 2710 | −110 | −63 | 22 | N |

| | | | | | | | | | | |
|---|---|---|---|---|---|---|---|---|---|---|
| ATOM | 1559 | CA | SER A | 592 | 21.994 | 14.639 | 82.554 | 1.00 | 20.81 | C |

| | | | | | | | | | | | |
|---|---|---|---|---|---|---|---|---|---|---|---|
| ANISOU | 1559 | CA | SER A | 592 | 2759 | 2333 | 2817 | 13 | −20 | 9 | C |

| | | | | | | | | | | |
|---|---|---|---|---|---|---|---|---|---|---|
| ATOM | 1560 | C | SER A | 592 | 22.749 | 13.574 | 83.332 | 1.00 | 22.20 | C |

| | | | | | | | | | | | |
|---|---|---|---|---|---|---|---|---|---|---|---|
| ANISOU | 1560 | C | SER A | 592 | 2799 | 2535 | 3102 | 47 | −126 | 53 | C |

| | | | | | | | | | | |
|---|---|---|---|---|---|---|---|---|---|---|
| ATOM | 1561 | O | SER A | 592 | 22.510 | 12.368 | 83.101 | 1.00 | 25.13 | O |

| | | | | | | | | | | | |
|---|---|---|---|---|---|---|---|---|---|---|---|
| ANISOU | 1561 | O | SER A | 592 | 3323 | 2530 | 3696 | −15 | −149 | 128 | O |

| | | | | | | | | | | |
|---|---|---|---|---|---|---|---|---|---|---|
| ATOM | 1562 | CB | SER A | 592 | 22.702 | 14.845 | 81.216 | 1.00 | 21.67 | C |

| | | | | | | | | | | | |
|---|---|---|---|---|---|---|---|---|---|---|---|
| ANISOU | 1562 | CB | SER A | 592 | 2931 | 2516 | 2785 | 90 | −47 | 78 | C |

| | | | | | | | | | | |
|---|---|---|---|---|---|---|---|---|---|---|
| ATOM | 1563 | OG | SER A | 592 | 24.093 | 15.104 | 81.330 | 1.00 | 24.49 | O |

| | | | | | | | | | | | |
|---|---|---|---|---|---|---|---|---|---|---|---|
| ANISOU | 1563 | OG | SER A | 592 | 3197 | 2506 | 3603 | −64 | 145 | 55 | O |

| | | | | | | | | | | |
|---|---|---|---|---|---|---|---|---|---|---|
| ATOM | 1564 | N | ALA A | 593 | 23.678 | 14.020 | 84.183 | 1.00 | 21.83 | N |

| | | | | | | | | | | | |
|---|---|---|---|---|---|---|---|---|---|---|---|
| ANISOU | 1564 | N | ALA A | 593 | 2899 | 2455 | 2939 | 53 | −171 | 177 | N |

| | | | | | | | | | | |
|---|---|---|---|---|---|---|---|---|---|---|
| ATOM | 1565 | CA | ALA A | 593 | 24.477 | 13.086 | 84.997 | 1.00 | 21.91 | C |

| | | | | | | | | | | | |
|---|---|---|---|---|---|---|---|---|---|---|---|
| ANISOU | 1565 | CA | ALA A | 593 | 2652 | 2553 | 3121 | 35 | −194 | 125 | C |

| | | | | | | | | | | |
|---|---|---|---|---|---|---|---|---|---|---|
| ATOM | 1566 | C | ALA A | 593 | 23.834 | 12.838 | 86.339 | 1.00 | 22.32 | C |

| | | | | | | | | | | | |
|---|---|---|---|---|---|---|---|---|---|---|---|
| ANISOU | 1566 | C | ALA A | 593 | 2870 | 2523 | 3088 | −153 | −238 | 194 | C |

| | | | | | | | | | | |
|---|---|---|---|---|---|---|---|---|---|---|
| ATOM | 1567 | O | ALA A | 593 | 24.409 | 12.103 | 87.194 | 1.00 | 23.63 | O |

| | | | | | | | | | | | |
|---|---|---|---|---|---|---|---|---|---|---|---|
| ANISOU | 1567 | O | ALA A | 593 | 3059 | 2628 | 3291 | −63 | −283 | 353 | O |

| | | | | | | | | | | |
|---|---|---|---|---|---|---|---|---|---|---|
| ATOM | 1568 | CB | ALA A | 593 | 25.857 | 13.747 | 85.245 | 1.00 | 21.21 | C |

| | | | | | | | | | | | |
|---|---|---|---|---|---|---|---|---|---|---|---|
| ANISOU | 1568 | CB | ALA A | 593 | 2674 | 2296 | 3088 | −20 | −107 | 20 | C |

| | | | | | | | | | | |
|---|---|---|---|---|---|---|---|---|---|---|
| ATOM | 1569 | N | ASP A | 594 | 22.661 | 13.384 | 86.612 | 1.00 | 23.59 | N |

| | | | | | | | | | | | |
|---|---|---|---|---|---|---|---|---|---|---|---|
| ANISOU | 1569 | N | ASP A | 594 | 3148 | 2496 | 3320 | −37 | −72 | 219 | N |

| | | | | | | | | | | |
|---|---|---|---|---|---|---|---|---|---|---|
| ATOM | 1570 | CA | ASP A | 594 | 21.996 | 13.318 | 87.887 | 1.00 | 24.82 | C |

| | | | | | | | | | | | |
|---|---|---|---|---|---|---|---|---|---|---|---|
| ANISOU | 1570 | CA | ASP A | 594 | 3161 | 2833 | 3436 | −159 | 2 | 159 | C |

| | | | | | | | | | | |
|---|---|---|---|---|---|---|---|---|---|---|
| ATOM | 1571 | C | ASP A | 594 | 22.938 | 13.707 | 89.016 | 1.00 | 25.08 | C |

| | | | | | | | | | | | |
|---|---|---|---|---|---|---|---|---|---|---|---|
| ANISOU | 1571 | C | ASP A | 594 | 3164 | 2864 | 3500 | −182 | −92 | 281 | C |

| | | | | | | | | | | |
|---|---|---|---|---|---|---|---|---|---|---|
| ATOM | 1572 | O | ASP A | 594 | 23.014 | 13.104 | 90.075 | 1.00 | 26.59 | O |

| | | | | | | | | | | | |
|---|---|---|---|---|---|---|---|---|---|---|---|
| ANISOU | 1572 | O | ASP A | 594 | 3536 | 3077 | 3491 | −207 | −123 | 433 | O |

| | | | | | | | | | | |
|---|---|---|---|---|---|---|---|---|---|---|
| ATOM | 1573 | CB | ASP A | 594 | 21.409 | 11.909 | 88.143 | 1.00 | 26.02 | C |

| | | | | | | | | | | | |
|---|---|---|---|---|---|---|---|---|---|---|---|
| ANISOU | 1573 | CB | ASP A | 594 | 3316 | 2893 | 3678 | −104 | 28 | 227 | C |

| | | | | | | | | | | |
|---|---|---|---|---|---|---|---|---|---|---|
| ATOM | 1574 | CG | ASP A | 594 | 20.158 | 11.698 | 87.293 | 1.00 | 27.94 | C |

| | | | | | | | | | | | |
|---|---|---|---|---|---|---|---|---|---|---|---|
| ANISOU | 1574 | CG | ASP A | 594 | 3320 | 3462 | 3836 | −248 | 24 | 205 | C |

| | | | | | | | | | | |
|---|---|---|---|---|---|---|---|---|---|---|
| ATOM | 1575 | OD1 | ASP A | 594 | 19.200 | 12.497 | 87.394 | 1.00 | 29.49 | O |

| | | | | | | | | | | | |
|---|---|---|---|---|---|---|---|---|---|---|---|
| ANISOU | 1575 | OD1 | ASP A | 594 | 3488 | 3625 | 4092 | −107 | −8 | 359 | O |

| | | | | | | | | | | |
|---|---|---|---|---|---|---|---|---|---|---|
| ATOM | 1576 | OD2 | ASP A | 594 | 20.100 | 10.701 | 86.573 | 1.00 | 30.04 | O |

| | | | | | | | | | | | |
|---|---|---|---|---|---|---|---|---|---|---|---|
| ANISOU | 1576 | OD2 | ASP A | 594 | 3527 | 4103 | 3786 | 62 | 64 | −73 | O |

| | | | | | | | | | | |
|---|---|---|---|---|---|---|---|---|---|---|
| ATOM | 1577 | N | ALA A | 595 | 23.658 | 14.827 | 88.873 | 1.00 | 23.58 | N |

| | | | | | | | | | | | |
|---|---|---|---|---|---|---|---|---|---|---|---|
| ANISOU | 1577 | N | ALA A | 595 | 3020 | 2665 | 3274 | −129 | −65 | 383 | N |

| | | | | | | | | | | |
|---|---|---|---|---|---|---|---|---|---|---|
| ATOM | 1578 | CA | ALA A | 595 | 24.601 | 15.336 | 89.833 | 1.00 | 24.45 | C |

| | | | | | | | | | | | |
|---|---|---|---|---|---|---|---|---|---|---|---|
| ANISOU | 1578 | CA | ALA A | 595 | 3147 | 2911 | 3233 | −117 | −137 | 381 | C |

| | | | | | | | | | | |
|---|---|---|---|---|---|---|---|---|---|---|
| ATOM | 1579 | C | ALA A | 595 | 24.059 | 16.537 | 90.580 | 1.00 | 25.46 | C |

| | | | | | | | | | | | |
|---|---|---|---|---|---|---|---|---|---|---|---|
| ANISOU | 1579 | C | ALA A | 595 | 3060 | 3203 | 3410 | −98 | 17 | 165 | C |

| | | | | | | | | | | |
|---|---|---|---|---|---|---|---|---|---|---|
| ATOM | 1580 | O | ALA A | 595 | 23.307 | 17.328 | 89.966 | 1.00 | 27.24 | O |

| | | | | | | | | | | | |
|---|---|---|---|---|---|---|---|---|---|---|---|
| ANTSOU | 1590 | O | ALA A | 595 | 3680 | 3042 | 3627 | −125 | −35 | 377 | O |

| | | | | | | | | | | |
|---|---|---|---|---|---|---|---|---|---|---|
| ATOM | 1581 | CB | ALA A | 595 | 25.823 | 15.778 | 88.991 | 1.00 | 23.95 | C |

| | | | | | | | | | | | |
|---|---|---|---|---|---|---|---|---|---|---|---|
| ANISOU | 1581 | CB | ALA A | 595 | 2852 | 2904 | 3344 | −40 | −101 | 74 | C |

| | | | | | | | | | | |
|---|---|---|---|---|---|---|---|---|---|---|
| ATOM | 1582 | N | PRO A | 596 | 24.375 | 16.744 | 91.844 | 1.00 | 26.95 | N |

| | | | | | | | | | | | |
|---|---|---|---|---|---|---|---|---|---|---|---|
| ANISOU | 1582 | N | PRO A | 596 | 3555 | 3310 | 3376 | −124 | 30 | 231 | N |

| | | | | | | | | | | |
|---|---|---|---|---|---|---|---|---|---|---|
| ATOM | 1583 | CA | PRO A | 596 | 23.931 | 17.860 | 92.645 | 1.00 | 27.99 | C |

| | | | | | | | | | | | |
|---|---|---|---|---|---|---|---|---|---|---|---|
| ANISOU | 1583 | CA | PRO A | 596 | 3785 | 3410 | 3438 | −94 | 23 | 227 | C |

| | | | | | | | | | | |
|---|---|---|---|---|---|---|---|---|---|---|
| ATOM | 1584 | C | PRO A | 596 | 24.365 | 19.165 | 91.958 | 1.00 | 27.48 | C |

| | | | | | | | | | | | |
|---|---|---|---|---|---|---|---|---|---|---|---|
| ANISOU | 1584 | C | PRO A | 596 | 3681 | 3320 | 3440 | −101 | −44 | 185 | C |

| | | | | | | | | | | |
|---|---|---|---|---|---|---|---|---|---|---|
| ATOM | 1585 | O | PRO A | 596 | 25.433 | 19.130 | 91.368 | 1.00 | 26.15 | O |

| | | | | | | | | | | | |
|---|---|---|---|---|---|---|---|---|---|---|---|
| ANISOU | 1585 | O | PRO A | 596 | 3607 | 3060 | 3269 | −96 | −133 | 283 | O |

| | | | | | | | | | | |
|---|---|---|---|---|---|---|---|---|---|---|
| ATOM | 1596 | CB | PRO A | 596 | 24.703 | 17.704 | 93.953 | 1.00 | 29.33 | C |

| | | | | | | | | | | | |
|---|---|---|---|---|---|---|---|---|---|---|---|
| ANISOU | 1586 | CB | PRO A | 596 | 3911 | 3724 | 3508 | 11 | −42 | 93 | C |

| | | | | | | | | | | |
|---|---|---|---|---|---|---|---|---|---|---|
| ATOM | 1587 | CG | PRO A | 596 | 24.951 | 16.225 | 94.025 | 1.00 | 29.64 | C |

| | | | | | | | | | | | |
|---|---|---|---|---|---|---|---|---|---|---|---|
| ANISOU | 1587 | CG | PRO A | 596 | 4003 | 3733 | 3526 | −84 | 35 | 123 | C |

| | | | | | | | | | | |
|---|---|---|---|---|---|---|---|---|---|---|
| ATOM | 1588 | CD | PRO A | 596 | 25.295 | 15.831 | 92.601 | 1.00 | 28.42 | C |

| | | | | | | | | | | | |
|---|---|---|---|---|---|---|---|---|---|---|---|
| ANISOU | 1588 | CD | PRO A | 596 | 3716 | 3566 | 3515 | 14 | −69 | 96 | C |

| | | | | | | | | | | |
|---|---|---|---|---|---|---|---|---|---|---|
| ATOM | 1589 | N | LEU A | 597 | 23.524 | 20.174 | 91.973 | 1.00 | 27.94 | N |

| | | | | | | | | | | | |
|---|---|---|---|---|---|---|---|---|---|---|---|
| ANISOU | 1589 | N | LEU A | 597 | 3929 | 3250 | 3436 | −51 | 33 | 213 | N |

| | | | | | | | | | | |
|---|---|---|---|---|---|---|---|---|---|---|
| ATOM | 1590 | CA | LEU A | 597 | 23.906 | 21.420 | 91.243 | 1.00 | 27.38 | C |

| | | | | | | | | | | | |
|---|---|---|---|---|---|---|---|---|---|---|---|
| ANISOU | 1590 | CA | LEU A | 597 | 3727 | 3383 | 3295 | −79 | 53 | 277 | C |

| | | | | | | | | | | |
|---|---|---|---|---|---|---|---|---|---|---|
| ATOM | 1591 | C | LEU A | 597 | 24.791 | 22.293 | 92.113 | 1.00 | 28.84 | C |

| | | | | | | | | | | | |
|---|---|---|---|---|---|---|---|---|---|---|---|
| ANISOU | 1591 | C | LEU A | 597 | 3977 | 3446 | 3536 | −157 | 79 | 201 | C |

| | | | | | | | | | | |
|---|---|---|---|---|---|---|---|---|---|---|
| ATOM | 1592 | O | LEU A | 597 | 24.268 | 23.207 | 92.758 | 1.00 | 29.55 | O |

| | | | | | | | | | | | |
|---|---|---|---|---|---|---|---|---|---|---|---|
| ANISOU | 1592 | O | LEU A | 597 | 4210 | 3436 | 3584 | −201 | 165 | 175 | O |

| | | | | | | | | | | |
|---|---|---|---|---|---|---|---|---|---|---|
| ATOM | 1593 | CB | LEU A | 597 | 22.596 | 22.126 | 90.814 | 1.00 | 25.56 | C |

| | | | | | | | | | | | |
|---|---|---|---|---|---|---|---|---|---|---|---|
| ANISOU | 1593 | CB | LEU A | 597 | 3600 | 3108 | 3003 | −102 | 223 | 184 | C |

| | | | | | | | | | | |
|---|---|---|---|---|---|---|---|---|---|---|
| ATOM | 1594 | CG | LEU A | 597 | 21.613 | 21.275 | 90.017 | 1.00 | 25.30 | C |

| | | | | | | | | | | | |
|---|---|---|---|---|---|---|---|---|---|---|---|
| ANISOU | 1594 | CG | LEU A | 597 | 3413 | 3191 | 3009 | 42 | 160 | 174 | C |

| | | | | | | | | | | |
|---|---|---|---|---|---|---|---|---|---|---|
| ATOM | 1595 | CD1 | LEU A | 597 | 20.386 | 22.064 | 99.594 | 1.00 | 23.99 | C |

| | | | | | | | | | | | |
|---|---|---|---|---|---|---|---|---|---|---|---|
| ANISOU | 1595 | CD1 | LEU A | 597 | 3367 | 2805 | 2944 | −20 | 230 | 185 | C |

| | | | | | | | | | | |
|---|---|---|---|---|---|---|---|---|---|---|
| ATOM | 1596 | CD2 | LEU A | 597 | 22.207 | 20.601 | 88.769 | 1.00 | 24.88 | C |

| | | | | | | | | | | | |
|---|---|---|---|---|---|---|---|---|---|---|---|
| ANISOU | 1596 | CD2 | LEU A | 597 | 3302 | 2962 | 3190 | 169 | 113 | 120 | C |

| | | | | | | | | | | |
|---|---|---|---|---|---|---|---|---|---|---|
| ATOM | 1597 | N | ASP A | 598 | 26.090 | 21.974 | 92.163 | 1.00 | 29.04 | N |

| | | | | | | | | | | | |
|---|---|---|---|---|---|---|---|---|---|---|---|
| ANISOU | 1597 | N | ASP A | 598 | 3977 | 3497 | 3560 | −191 | 14 | 369 | N |

| | | | | | | | | | | |
|---|---|---|---|---|---|---|---|---|---|---|
| ATOM | 1598 | CA | ASP A | 598 | 26.990 | 22.694 | 93.045 | 1.00 | 31.19 | C |

| | | | | | | | | | | | |
|---|---|---|---|---|---|---|---|---|---|---|---|
| ANISOU | 1598 | CA | ASP A | 598 | 3988 | 4002 | 3862 | −191 | −48 | 194 | C |

| | | | | | | | | | | |
|---|---|---|---|---|---|---|---|---|---|---|
| ATOM | 1599 | C | ASP A | 598 | 28.111 | 23.413 | 92.290 | 1.00 | 29.95 | C |

| | | | | | | | | | | | |
|---|---|---|---|---|---|---|---|---|---|---|---|
| ANISOU | 1599 | C | ASP A | 598 | 3848 | 3913 | 3620 | −147 | −65 | 214 | C |

| | | | | | | | | | | |
|---|---|---|---|---|---|---|---|---|---|---|
| ATOM | 1600 | O | ASP A | 598 | 29.121 | 23.819 | 92.878 | 1.00 | 31.27 | O |

| | | | | | | | | | | | |
|---|---|---|---|---|---|---|---|---|---|---|---|
| ANISOU | 1600 | O | ASP A | 598 | 4012 | 4046 | 3825 | −244 | −108 | 309 | O |

| | | | | | | | | | | |
|---|---|---|---|---|---|---|---|---|---|---|
| ATOM | 1601 | CB | ASP A | 598 | 27.572 | 21.752 | 94.100 | 1.00 | 32.87 | C |

| | | | | | | | | | | | |
|---|---|---|---|---|---|---|---|---|---|---|---|
| ANISOU | 1601 | CB | ASP A | 598 | 4345 | 4096 | 4047 | −52 | −63 | 262 | C |

| | | | | | | | | | | |
|---|---|---|---|---|---|---|---|---|---|---|
| ATOM | 1602 | CG | ASP A | 598 | 28.265 | 20.527 | 93.570 | 1.00 | 35.53 | C |

| | | | | | | | | | | | |
|---|---|---|---|---|---|---|---|---|---|---|---|
| ANISOU | 1602 | CG | ASP A | 598 | 4633 | 4443 | 4423 | 47 | 15 | 30 | C |

| | | | | | | | | | | |
|---|---|---|---|---|---|---|---|---|---|---|
| ATOM | 1603 | OD1 | ASP A | 598 | 28.549 | 20.335 | 92.374 | 1.00 | 34.43 | O |

| | | | | | | | | | | | |
|---|---|---|---|---|---|---|---|---|---|---|---|
| ANISOU | 1603 | OD1 | ASP A | 598 | 4502 | 4297 | 4285 | 19 | −53 | 249 | O |

| | | | | | | | | | | |
|---|---|---|---|---|---|---|---|---|---|---|
| ATOM | 1604 | OD2 | ASP A | 598 | 28.529 | 19.643 | 94.444 | 1.00 | 37.50 | O |

| | | | | | | | | | | | |
|---|---|---|---|---|---|---|---|---|---|---|---|
| ANISOU | 1604 | OD2 | ASP A | 598 | 4992 | 4669 | 4588 | 135 | −79 | 138 | O |

| | | | | | | | | | | |
|---|---|---|---|---|---|---|---|---|---|---|
| ATOM | 1605 | N | PHE A | 599 | 27.917 | 23.551 | 90.992 | 1.00 | 26.85 | N |

| | | | | | | | | | | | |
|---|---|---|---|---|---|---|---|---|---|---|---|
| ANISOU | 1605 | N | PHE A | 599 | 3225 | 3441 | 3536 | −271 | −83 | 251 | N |

| | | | | | | | | | | |
|---|---|---|---|---|---|---|---|---|---|---|
| ATOM | 1606 | CA | PHE A | 599 | 28.898 | 24.313 | 90.190 | 1.00 | 24.83 | C |

| | | | | | | | | | | | |
|---|---|---|---|---|---|---|---|---|---|---|---|
| ANISOU | 1606 | CA | PHE A | 599 | 3098 | 3226 | 3109 | −128 | −149 | 148 | C |

| | | | | | | | | | | |
|---|---|---|---|---|---|---|---|---|---|---|
| ATOM | 1607 | C | PHE A | 599 | 28.790 | 25.772 | 90.612 | 1.00 | 25.16 | C |

| | | | | | | | | | | | |
|---|---|---|---|---|---|---|---|---|---|---|---|
| ANISOU | 1607 | C | PHE A | 599 | 3151 | 3265 | 3146 | −82 | −21 | 159 | C |

| | | | | | | | | | | |
|---|---|---|---|---|---|---|---|---|---|---|
| ATOM | 1608 | O | PHE A | 599 | 27.858 | 26.255 | 91.265 | 1.00 | 25.44 | O |

| | | | | | | | | | | | |
|---|---|---|---|---|---|---|---|---|---|---|---|
| ANISOU | 1608 | O | PHE A | 599 | 3136 | 3311 | 3220 | −258 | 27 | 124 | O |

| | | | | | | | | | | |
|---|---|---|---|---|---|---|---|---|---|---|
| ATOM | 1609 | CB | PHE A | 599 | 28.581 | 24.102 | 88.707 | 1.00 | 23.88 | C |

| | | | | | | | | | | | |
|---|---|---|---|---|---|---|---|---|---|---|---|
| ANISOU | 1609 | CB | PHE A | 599 | 3038 | 2995 | 3041 | −53 | 54 | 167 | C |

| | | | | | | | | | | |
|---|---|---|---|---|---|---|---|---|---|---|
| ATOM | 1610 | CG | PHE A | 599 | 27.157 | 24.337 | 88.304 | 1.00 | 23.04 | C |

| | | | | | | | | | | | |
|---|---|---|---|---|---|---|---|---|---|---|---|
| ANISOU | 1610 | CG | PHE A | 599 | 3002 | 2703 | 3048 | −134 | 37 | 107 | C |

| | | | | | | | | | | |
|---|---|---|---|---|---|---|---|---|---|---|
| ATOM | 1611 | CD1 | PHE A | 599 | 26.700 | 25.624 | 88.119 | 1.00 | 22.28 | C |

| | | | | | | | | | | | |
|---|---|---|---|---|---|---|---|---|---|---|---|
| ANISOU | 1611 | CD1 | PHE A | 599 | 2986 | 2648 | 2832 | −161 | 6 | 225 | C |

| | | | | | | | | | | |
|---|---|---|---|---|---|---|---|---|---|---|
| ATOM | 1612 | CD2 | PHE A | 599 | 26.248 | 23.294 | 88.072 | 1.00 | 21.80 | C |

| | | | | | | | | | | | |
|---|---|---|---|---|---|---|---|---|---|---|---|
| ANISOU | 1612 | CD2 | PHE A | 598 | 2787 | 2692 | 2805 | −102 | −18 | 102 | C |

| | | | | | | | | | | |
|---|---|---|---|---|---|---|---|---|---|---|
| ATOM | 1613 | CE1 | PHE A | 599 | 25.409 | 25.882 | 87.742 | 1.00 | 22.53 | C |

| | | | | | | | | | | | |
|---|---|---|---|---|---|---|---|---|---|---|---|
| ANISOU | 1613 | CE1 | PHE A | 599 | 2902 | 2495 | 3162 | −98 | 48 | 89 | C |

| | | | | | | | | | | |
|---|---|---|---|---|---|---|---|---|---|---|
| ATOM | 1614 | CE2 | PHE A | 599 | 24.959 | 23.554 | 87.677 | 1.00 | 21.61 | C |

| | | | | | | | | | | | |
|---|---|---|---|---|---|---|---|---|---|---|---|
| ANISOU | 1614 | CE2 | PHE A | 599 | 2819 | 2598 | 2793 | 17 | 167 | 58 | C |

| | | | | | | | | | | |
|---|---|---|---|---|---|---|---|---|---|---|
| ATOM | 1615 | CZ | PHE A | 599 | 24.500 | 24.850 | 87.490 | 1.00 | 22.64 | C |

| | | | | | | | | | | | |
|---|---|---|---|---|---|---|---|---|---|---|---|
| ANISOU | 1615 | CZ | PHE A | 599 | 3010 | 2566 | 3024 | −119 | −25 | 95 | C |

| | | | | | | | | | | |
|---|---|---|---|---|---|---|---|---|---|---|
| ATOM | 1616 | N | ASP A | 600 | 29.798 | 26.540 | 90.164 | 1.00 | 24.19 | N |

| | | | | | | | | | | | |
|---|---|---|---|---|---|---|---|---|---|---|---|
| ANISOU | 1616 | N | ASP A | 600 | 3133 | 3051 | 3009 | −160 | 11 | 107 | N |

| | | | | | | | | | | |
|---|---|---|---|---|---|---|---|---|---|---|
| ATOM | 1617 | CA | ASP A | 600 | 29.844 | 27.974 | 90.466 | 1.00 | 25.05 | C |

| | | | | | | | | | | | |
|---|---|---|---|---|---|---|---|---|---|---|---|
| ANISOU | 1617 | CA | ASP A | 600 | 3165 | 3116 | 3237 | −82 | 89 | 28 | C |

| | | | | | | | | | | |
|---|---|---|---|---|---|---|---|---|---|---|
| ATOM | 1618 | C | ASP A | 600 | 30.354 | 28.804 | 89.316 | 1.00 | 23.68 | C |

| | | | | | | | | | | | |
|---|---|---|---|---|---|---|---|---|---|---|---|
| ANISOU | 1618 | C | ASP A | 600 | 3167 | 2819 | 3013 | 3 | −47 | −52 | C |

| | | | | | | | | | | |
|---|---|---|---|---|---|---|---|---|---|---|
| ATOM | 1619 | O | ASP A | 600 | 30.542 | 28.313 | 88.214 | 1.00 | 21.71 | O |

| | | | | | | | | | | | |
|---|---|---|---|---|---|---|---|---|---|---|---|
| ANISOU | 1619 | O | ASP A | 600 | 2866 | 2354 | 3030 | −112 | 188 | 21 | O |

| | | | | | | | | | | |
|---|---|---|---|---|---|---|---|---|---|---|
| ATOM | 1620 | CB | ASP A | 600 | 30.720 | 28.231 | 91.701 | 1.00 | 26.96 | C |

| | | | | | | | | | | | |
|---|---|---|---|---|---|---|---|---|---|---|---|
| ANISOU | 1620 | CB | ASP A | 600 | 3507 | 3477 | 3260 | −144 | −28 | 64 | C |

| | | | | | | | | | | |
|---|---|---|---|---|---|---|---|---|---|---|
| ATOM | 1621 | CG | ASP A | 600 | 32.153 | 27.782 | 91.458 | 1.00 | 29.38 | C |

| | | | | | | | | | | | |
|---|---|---|---|---|---|---|---|---|---|---|---|
| ANISOU | 1621 | CG | ASP A | 600 | 3562 | 3804 | 3796 | −31 | −28 | −17 | C |

| | | | | | | | | | | |
|---|---|---|---|---|---|---|---|---|---|---|
| ATOM | 1622 | OD1 | ASP A | 600 | 32.679 | 27.806 | 90.322 | 1.00 | 27.07 | O |

| | | | | | | | | | | | |
|---|---|---|---|---|---|---|---|---|---|---|---|
| ANISOU | 1622 | OD1 | ASP A | 600 | 3206 | 3273 | 3808 | −335 | −116 | 151 | O |

| | | | | | | | | | | |
|---|---|---|---|---|---|---|---|---|---|---|
| ATOM | 1623 | OD2 | ASP A | 600 | 32.835 | 27.340 | 92.425 | 1.00 | 33.08 | O |

| | | | | | | | | | | | |
|---|---|---|---|---|---|---|---|---|---|---|---|
| ANISOU | 1623 | OD2 | ASP A | 600 | 4285 | 4283 | 4001 | −63 | −211 | 224 | O |

| | | | | | | | | | | |
|---|---|---|---|---|---|---|---|---|---|---|
| ATOM | 1624 | N | TYR A | 601 | 30.623 | 30.104 | 89.581 | 1.00 | 23.00 | N |

| | | | | | | | | | | | |
|---|---|---|---|---|---|---|---|---|---|---|---|
| ANISOU | 1624 | N | TYR A | 601 | 2881 | 2784 | 3074 | −181 | 6 | −13 | N |

| | | | | | | | | | | |
|---|---|---|---|---|---|---|---|---|---|---|
| ATOM | 1625 | CA | TYR A | 601 | 30.998 | 30.987 | 88.477 | 1.00 | 23.93 | C |

| | | | | | | | | | | | |
|---|---|---|---|---|---|---|---|---|---|---|---|
| ANISOU | 1625 | CA | TYR A | 601 | 3108 | 2869 | 3115 | −23 | 112 | 4 | C |

| | | | | | | | | | | |
|---|---|---|---|---|---|---|---|---|---|---|
| ATOM | 1626 | C | TYR A | 601 | 32.373 | 30.687 | 87.901 | 1.00 | 22.53 | C |

| | | | | | | | | | | | |
|---|---|---|---|---|---|---|---|---|---|---|---|
| ANISOU | 1626 | C | TYR A | 601 | 2963 | 2527 | 3070 | −121 | −1 | −65 | C |

| | | | | | | | | | | |
|---|---|---|---|---|---|---|---|---|---|---|
| ATOM | 1627 | O | TYR A | 601 | 32.586 | 31.070 | 86.749 | 1.00 | 22.09 | O |

| | | | | | | | | | | | |
|---|---|---|---|---|---|---|---|---|---|---|---|
| ANISOU | 1627 | O | TYR A | 601 | 2989 | 2358 | 3045 | −232 | −155 | −181 | O |

| | | | | | | | | | | |
|---|---|---|---|---|---|---|---|---|---|---|
| ATOM | 1628 | CB | TYR A | 601 | 30.915 | 32.464 | 88.854 | 1.00 | 25.47 | C |

| | | | | | | | | | | | |
|---|---|---|---|---|---|---|---|---|---|---|---|
| ANISOU | 1628 | CB | TYR A | 601 | 3374 | 2902 | 3402 | 58 | 169 | −11 | C |

| | | | | | | | | | | |
|---|---|---|---|---|---|---|---|---|---|---|
| ATOM | 1629 | CG | TYR A | 601 | 32.106 | 32.960 | 89.642 | 1.00 | 27.42 | C |

| | | | | | | | | | | | |
|---|---|---|---|---|---|---|---|---|---|---|---|
| ANISOU | 1629 | CG | TYR A | 601 | 3456 | 3295 | 3669 | 53 | 74 | −101 | C |

| | | | | | | | | | | |
|---|---|---|---|---|---|---|---|---|---|---|
| ATOM | 1630 | CD1 | TYR A | 601 | 33.002 | 33.759 | 88.923 | 1.00 | 28.68 | C |

| | | | | | | | | | | | |
|---|---|---|---|---|---|---|---|---|---|---|---|
| ANISOU | 1630 | CD1 | TYR A | 601 | 3624 | 3501 | 3772 | −29 | 125 | −38 | C |

| | | | | | | | | | | |
|---|---|---|---|---|---|---|---|---|---|---|
| ATOM | 1631 | CD2 | TYR A | 601 | 32.356 | 32.704 | 90.971 | 1.00 | 27.34 | C |

| | | | | | | | | | | | |
|---|---|---|---|---|---|---|---|---|---|---|---|
| ANISOU | 1631 | CD2 | TYR A | 601 | 3461 | 3216 | 3713 | −24 | 49 | −77 | C |

| | | | | | | | | | | |
|---|---|---|---|---|---|---|---|---|---|---|
| ATOM | 1632 | CE1 | TYR A | 601 | 34.127 | 34.265 | 89.545 | 1.00 | 31.22 | C |

| | | | | | | | | | | | |
|---|---|---|---|---|---|---|---|---|---|---|---|
| ANISOU | 1632 | CE1 | TYR A | 601 | 3922 | 3822 | 4117 | −95 | −46 | −82 | C |

| | | | | | | | | | | |
|---|---|---|---|---|---|---|---|---|---|---|
| ATOM | 1633 | CE2 | TYR A | 601 | 33.486 | 33.223 | 91.622 | 1.00 | 30.06 | C |

| | | | | | | | | | | | |
|---|---|---|---|---|---|---|---|---|---|---|---|
| ANISOU | 1633 | CE2 | TYR A | 601 | 3700 | 3660 | 4061 | −59 | −82 | −168 | C |

| | | | | | | | | | | |
|---|---|---|---|---|---|---|---|---|---|---|
| ATOM | 1634 | CZ | TYR A | 601 | 34.345 | 34.003 | 90.870 | 1.00 | 31.10 | C |

| | | | | | | | | | | | |
|---|---|---|---|---|---|---|---|---|---|---|---|
| ANISOU | 1634 | CZ | TYR A | 601 | 3948 | 3810 | 4059 | −102 | −12 | −80 | C |

| | | | | | | | | | | |
|---|---|---|---|---|---|---|---|---|---|---|
| ATOM | 1635 | OH | TYR A | 601 | 35.489 | 34.534 | 91.430 | 1.00 | 34.40 | O |

| | | | | | | | | | | | |
|---|---|---|---|---|---|---|---|---|---|---|---|
| ANISOU | 1635 | OH | TYR A | 601 | 4128 | 4249 | 4695 | −335 | −50 | −160 | O |

| | | | | | | | | | | |
|---|---|---|---|---|---|---|---|---|---|---|
| ATOM | 1636 | N | LYS A | 602 | 33.235 | 30.034 | 88.683 | 1.00 | 22.59 | N |

| | | | | | | | | | | | |
|---|---|---|---|---|---|---|---|---|---|---|---|
| ANISOU | 1636 | N | LYS A | 602 | 2780 | 2694 | 3110 | −50 | 15 | −34 | N |

| | | | | | | | | | | |
|---|---|---|---|---|---|---|---|---|---|---|
| ATOM | 1637 | CA | LYS A | 602 | 34.536 | 29.637 | 88.115 | 1.00 | 23.43 | C |

| | | | | | | | | | | | |
|---|---|---|---|---|---|---|---|---|---|---|---|
| ANISOU | 1637 | CA | LYS A | 602 | 2910 | 2822 | 3171 | −66 | −34 | −87 | C |

| | | | | | | | | | | |
|---|---|---|---|---|---|---|---|---|---|---|
| ATOM | 1638 | C | LYS A | 602 | 34.320 | 28.485 | 87.131 | 1.00 | 21.11 | C |

| | | | | | | | | | | | |
|---|---|---|---|---|---|---|---|---|---|---|---|
| ANISOU | 1638 | C | LYS A | 602 | 2551 | 2625 | 2844 | −32 | −93 | 124 | C |

| | | | | | | | | | | |
|---|---|---|---|---|---|---|---|---|---|---|
| ATOM | 1639 | O | LYS A | 602 | 34.974 | 28.404 | 86.096 | 1.00 | 20.00 | O |

| | | | | | | | | | | | |
|---|---|---|---|---|---|---|---|---|---|---|---|
| ANISOU | 1639 | O | LYS A | 602 | 2793 | 2215 | 2593 | −110 | −270 | 161 | O |

| | | | | | | | | | | |
|---|---|---|---|---|---|---|---|---|---|---|
| ATOM | 1640 | CB | LYS A | 602 | 35.501 | 29.259 | 89.238 | 1.00 | 25.08 | C |

| | | | | | | | | | | | |
|---|---|---|---|---|---|---|---|---|---|---|---|
| ANISOU | 1640 | CB | LYS A | 602 | 3216 | 3205 | 3108 | 57 | −82 | 42 | C |

| | | | | | | | | | | |
|---|---|---|---|---|---|---|---|---|---|---|
| ATOM | 1641 | CG | LYS A | 602 | 35.763 | 30.422 | 90.178 | 1.00 | 28.29 | C |

| | | | | | | | | | | | |
|---|---|---|---|---|---|---|---|---|---|---|---|
| ANISOU | 1641 | CG | LYS A | 602 | 3679 | 3448 | 3622 | −80 | −6 | −182 | C |

| | | | | | | | | | | |
|---|---|---|---|---|---|---|---|---|---|---|
| ATOM | 1642 | CD | LYS A | 602 | 36.742 | 30.059 | 91.294 | 1.00 | 31.15 | C |

| | | | | | | | | | | | |
|---|---|---|---|---|---|---|---|---|---|---|---|
| ANISOU | 1642 | CD | LYS A | 602 | 3996 | 3959 | 3880 | 6 | −124 | 103 | C |

| | | | | | | | | | | |
|---|---|---|---|---|---|---|---|---|---|---|
| ATOM | 1643 | CE | LYS A | 602 | 36.820 | 31.260 | 92.239 | 1.00 | 34.67 | C |

| | | | | | | | | | | | |
|---|---|---|---|---|---|---|---|---|---|---|---|
| ANISOU | 1643 | CE | LYS A | 602 | 4624 | 4288 | 4261 | −68 | −69 | −124 | C |

| | | | | | | | | | | |
|---|---|---|---|---|---|---|---|---|---|---|
| ATOM | 1644 | NZ | LYS A | 602 | 37.947 | 31.103 | 93.200 | 1.00 | 36.24 | N |

| | | | | | | | | | | | |
|---|---|---|---|---|---|---|---|---|---|---|---|
| ANISO | 1644 | NZ | LYS A | 602 | 4693 | 4587 | 4488 | −2 | −158 | −7 | N |

| | | | | | | | | | | |
|---|---|---|---|---|---|---|---|---|---|---|
| ATOM | 1645 | N | ASP A | 603 | 33.376 | 27.576 | 87.467 | 1.00 | 21.49 | N |

| | | | | | | | | | | | |
|---|---|---|---|---|---|---|---|---|---|---|---|
| ANISOU | 1645 | N | ASP A | 603 | 2627 | 2580 | 2958 | −40 | −213 | 32 | N |

| | | | | | | | | | | |
|---|---|---|---|---|---|---|---|---|---|---|
| ATOM | 1646 | CA | ASP A | 603 | 33.041 | 26.540 | 86.512 | 1.00 | 20.70 | C |

| | | | | | | | | | | | |
|---|---|---|---|---|---|---|---|---|---|---|---|
| ANISOU | 1646 | CA | ASP A | 603 | 2503 | 2580 | 2783 | −64 | −94 | 64 | C |

| | | | | | | | | | | |
|---|---|---|---|---|---|---|---|---|---|---|
| ATOM | 1647 | C | ASP A | 603 | 32.507 | 27.199 | 85.242 | 1.00 | 19.84 | C |

| | | | | | | | | | | | |
|---|---|---|---|---|---|---|---|---|---|---|---|
| ANISOU | 1647 | C | ASP A | 603 | 2374 | 2362 | 2802 | −9 | −39 | 58 | C |

| | | | | | | | | | | |
|---|---|---|---|---|---|---|---|---|---|---|
| ATOM | 1648 | O | ASP A | 603 | 32.747 | 26.694 | 84.169 | 1.00 | 20.25 | O |

| | | | | | | | | | | | |
|---|---|---|---|---|---|---|---|---|---|---|---|
| ANISOU | 1648 | O | ASP A | 603 | 2571 | 2168 | 2955 | 22 | −34 | 10 | O |

| | | | | | | | | | | |
|---|---|---|---|---|---|---|---|---|---|---|
| ATOM | 1649 | CB | ASP A | 603 | 31.939 | 25.602 | 87.073 | 1.00 | 21.75 | C |

| | | | | | | | | | | | |
|---|---|---|---|---|---|---|---|---|---|---|---|
| ANISOU | 1649 | CB | ASP A | 603 | 2753 | 2565 | 2946 | −117 | 12 | 58 | C |

| | | | | | | | | | | |
|---|---|---|---|---|---|---|---|---|---|---|
| ATOM | 1650 | CG | ASP A | 603 | 32.473 | 24.814 | 88.259 | 1.00 | 24.95 | C |

| | | | | | | | | | | | |
|---|---|---|---|---|---|---|---|---|---|---|---|
| ANISOU | 1650 | CG | ASP A | 603 | 3244 | 3090 | 3146 | 21 | −102 | 103 | C |

| | | | | | | | | | | |
|---|---|---|---|---|---|---|---|---|---|---|
| ATOM | 1651 | OD1 | ASP A | 603 | 33.504 | 24.124 | 88.067 | 1.00 | 26.87 | O |

| | | | | | | | | | | | |
|---|---|---|---|---|---|---|---|---|---|---|---|
| ANISOU | 1651 | OD1 | ASP A | 603 | 3238 | 3253 | 3718 | 83 | −119 | 288 | O |

| | | | | | | | | | | |
|---|---|---|---|---|---|---|---|---|---|---|
| ATOM | 1652 | OD2 | ASP A | 603 | 31.931 | 24.832 | 89.375 | 1.00 | 27.25 | O |

| | | | | | | | | | | | |
|---|---|---|---|---|---|---|---|---|---|---|---|
| ANISOU | 1652 | OD2 | ASP A | 603 | 3427 | 3575 | 3351 | −29 | 37 | 71 | O |

| | | | | | | | | | | |
|---|---|---|---|---|---|---|---|---|---|---|
| ATOM | 1653 | N | ALA A | 604 | 31.718 | 28.290 | 85.353 | 1.00 | 19.54 | N |

| | | | | | | | | | | | |
|---|---|---|---|---|---|---|---|---|---|---|---|
| ANISOU | 1653 | N | ALA A | 604 | 2625 | 2177 | 2620 | 7 | −79 | 4 | N |

| | | | | | | | | | | |
|---|---|---|---|---|---|---|---|---|---|---|
| ATOM | 1654 | CA | ALA A | 604 | 31.201 | 28.929 | 84.157 | 1.00 | 19.18 | C |

| | | | | | | | | | | | |
|---|---|---|---|---|---|---|---|---|---|---|---|
| ANISOU | 1654 | CA | ALA A | 604 | 2317 | 2340 | 2631 | 244 | 25 | 23 | C |

| | | | | | | | | | | |
|---|---|---|---|---|---|---|---|---|---|---|
| ATOM | 1655 | C | ALA A | 604 | 32.294 | 29.466 | 83.253 | 1.00 | 18.27 | C |

| | | | | | | | | | | | |
|---|---|---|---|---|---|---|---|---|---|---|---|
| ANISOU | 1655 | C | ALA A | 604 | 2402 | 2110 | 2431 | 113 | −28 | −5 | C |

| | | | | | | | | | | |
|---|---|---|---|---|---|---|---|---|---|---|
| ATOM | 1656 | O | ALA A | 604 | 32.274 | 29.280 | 82.024 | 1.00 | 17.93 | O |

| | | | | | | | | | | | |
|---|---|---|---|---|---|---|---|---|---|---|---|
| ANISOU | 1656 | O | ALA A | 604 | 2357 | 2005 | 2452 | 9 | −136 | 10 | O |

| | | | | | | | | | | |
|---|---|---|---|---|---|---|---|---|---|---|
| ATOM | 1657 | CB | ALA A | 604 | 30.227 | 30.061 | 84.549 | 1.00 | 18.43 | C |

| | | | | | | | | | | | |
|---|---|---|---|---|---|---|---|---|---|---|---|
| ANISOU | 1657 | CB | ALA A | 604 | 2495 | 2034 | 2473 | 172 | 217 | −85 | C |

| | | | | | | | | | | |
|---|---|---|---|---|---|---|---|---|---|---|
| ATOM | 1658 | N | ILE A | 605 | 33.357 | 30.040 | 83.881 | 1.00 | 18.09 | N |

| | | | | | | | | | | | |
|---|---|---|---|---|---|---|---|---|---|---|---|
| ANISOU | 1658 | N | ILE A | 605 | 2206 | 2018 | 2649 | 84 | −200 | 101 | N |

| | | | | | | | | | | |
|---|---|---|---|---|---|---|---|---|---|---|
| ATOM | 1659 | CA | ILE A | 605 | 34.524 | 30.487 | 83.090 | 1.00 | 20.05 | C |

| | | | | | | | | | | | |
|---|---|---|---|---|---|---|---|---|---|---|---|
| ANISOU | 1659 | CA | ILE A | 605 | 2547 | 2479 | 2593 | −41 | −65 | 99 | C |

| | | | | | | | | | | |
|---|---|---|---|---|---|---|---|---|---|---|
| ATOM | 1660 | C | ILE A | 605 | 35.181 | 29.333 | 82.349 | 1.00 | 20.12 | C |

| | | | | | | | | | | | |
|---|---|---|---|---|---|---|---|---|---|---|---|
| ANISOU | 1660 | C | ILE A | 605 | 2441 | 2666 | 2537 | −33 | −44 | 42 | C |

| | | | | | | | | | | |
|---|---|---|---|---|---|---|---|---|---|---|
| ATOM | 1661 | O | ILE A | 605 | 35.406 | 29.342 | 81.123 | 1.00 | 19.13 | O |

| | | | | | | | | | | | |
|---|---|---|---|---|---|---|---|---|---|---|---|
| ANISOU | 1661 | O | ILE A | 605 | 2040 | 2732 | 2496 | −99 | −53 | 308 | O |

| | | | | | | | | | | |
|---|---|---|---|---|---|---|---|---|---|---|
| ATOM | 1662 | CB | ILE A | 605 | 35.566 | 31.179 | 84.004 | 1.00 | 22.12 | C |

| | | | | | | | | | | | |
|---|---|---|---|---|---|---|---|---|---|---|---|
| ANISOU | 1662 | CB | ILE A | 605 | 2722 | 2902 | 2781 | −53 | −161 | −18 | C |

| | | | | | | | | | | |
|---|---|---|---|---|---|---|---|---|---|---|
| ATOM | 1663 | CG1 | ILE A | 605 | 34.967 | 32.459 | 84.578 | 1.00 | 23.89 | C |

| | | | | | | | | | | | |
|---|---|---|---|---|---|---|---|---|---|---|---|
| ANISOU | 1663 | CG1 | ILE A | 605 | 3070 | 2951 | 3055 | 7 | −153 | −50 | C |

| | | | | | | | | | | |
|---|---|---|---|---|---|---|---|---|---|---|
| ATOM | 1664 | CG2 | ILE A | 605 | 36.839 | 31.425 | 83.206 | 1.00 | 22.71 | C |

| | | | | | | | | | | | |
|---|---|---|---|---|---|---|---|---|---|---|---|
| ANISOU | 1664 | CG2 | ILE A | 605 | 2780 | 2808 | 3039 | −145 | −60 | 34 | C |

| | | | | | | | | | | |
|---|---|---|---|---|---|---|---|---|---|---|
| ATOM | 1665 | CD1 | ILE A | 605 | 35.732 | 32.980 | 85.815 | 1.00 | 25.66 | C |

| | | | | | | | | | | | |
|---|---|---|---|---|---|---|---|---|---|---|---|
| ANISOU | 1665 | CD1 | ILE A | 605 | 3420 | 3243 | 3087 | −53 | −191 | −125 | C |

| | | | | | | | | | | |
|---|---|---|---|---|---|---|---|---|---|---|
| ATOM | 1666 | N | ARG A | 606 | 35.368 | 28.196 | 83.040 | 1.00 | 20.12 | N |

| | | | | | | | | | | | |
|---|---|---|---|---|---|---|---|---|---|---|---|
| ANISOU | 1666 | N | ARG A | 606 | 2448 | 2560 | 2637 | −79 | −51 | 69 | N |

| | | | | | | | | | | |
|---|---|---|---|---|---|---|---|---|---|---|
| ATOM | 1667 | CA | ARG A | 606 | 35.949 | 27.000 | 82.408 | 1.00 | 20.36 | C |

| | | | | | | | | | | | |
|---|---|---|---|---|---|---|---|---|---|---|---|
| ANISOU | 1667 | CA | ARG A | 606 | 2577 | 2493 | 2667 | 9 | −50 | 121 | C |

| | | | | | | | | | | |
|---|---|---|---|---|---|---|---|---|---|---|
| ATOM | 1668 | C | ARG A | 606 | 35.010 | 26.402 | 81.374 | 1.00 | 19.06 | C |

| | | | | | | | | | | | |
|---|---|---|---|---|---|---|---|---|---|---|---|
| ANISOU | 1668 | C | ARG A | 606 | 2646 | 2122 | 2474 | 113 | −19 | 254 | C |

| | | | | | | | | | | |
|---|---|---|---|---|---|---|---|---|---|---|
| ATOM | 1669 | O | ARG A | 606 | 35.447 | 25.822 | 80.366 | 1.00 | 19.14 | O |

| | | | | | | | | | | | |
|---|---|---|---|---|---|---|---|---|---|---|---|
| ANISOU | 1669 | O | ARG A | 606 | 2476 | 2306 | 2493 | 98 | −42 | 176 | O |

| | | | | | | | | | | |
|---|---|---|---|---|---|---|---|---|---|---|
| ATOM | 1670 | CB | ARG A | 606 | 36.295 | 25.912 | 83.473 | 1.00 | 22.72 | C |

| | | | | | | | | | | | |
|---|---|---|---|---|---|---|---|---|---|---|---|
| ANISOU | 1670 | CB | ARG A | 606 | 2998 | 2540 | 3096 | 272 | −55 | 189 | C |

| | | | | | | | | | | |
|---|---|---|---|---|---|---|---|---|---|---|
| ATOM | 1671 | CG | ARG A | 606 | 37.308 | 26.410 | 84.479 | 1.00 | 26.58 | C |

| | | | | | | | | | | | |
|---|---|---|---|---|---|---|---|---|---|---|---|
| ANISOU | 1671 | CG | ARG A | 606 | 3415 | 3256 | 3430 | 66 | −151 | 11 | C |

| | | | | | | | | | | |
|---|---|---|---|---|---|---|---|---|---|---|
| ATOM | 1672 | CD | ARG A | 606 | 38.035 | 25.267 | 85.256 | 1.00 | 31.19 | C |

| | | | | | | | | | | | |
|---|---|---|---|---|---|---|---|---|---|---|---|
| ANISOU | 1672 | CD | ARG A | 606 | 3774 | 3958 | 4120 | 416 | −263 | 239 | C |

| | | | | | | | | | | |
|---|---|---|---|---|---|---|---|---|---|---|
| ATOM | 1673 | NE | ARG A | 606 | 38.633 | 25.996 | 86.406 | 1.00 | 36.26 | N |

| | | | | | | | | | | | |
|---|---|---|---|---|---|---|---|---|---|---|---|
| ANISOU | 1673 | NE | ARG A | 606 | 4700 | 4591 | 4486 | 44 | −222 | −63 | N |

| | | | | | | | | | | |
|---|---|---|---|---|---|---|---|---|---|---|
| ATOM | 1674 | CZ | ARG A | 606 | 38.021 | 26.191 | 87.579 | 1.00 | 38.29 | C |

| | | | | | | | | | | | |
|---|---|---|---|---|---|---|---|---|---|---|---|
| ANISOU | 1674 | CZ | ARG A | 606 | 4854 | 4946 | 4750 | 102 | 8 | −24 | C |

| | | | | | | | | | | |
|---|---|---|---|---|---|---|---|---|---|---|
| ATOM | 1675 | NH1 | ARG A | 606 | 36.817 | 25.729 | 87.869 | 1.00 | 37.81 | N |

| | | | | | | | | | | | |
|---|---|---|---|---|---|---|---|---|---|---|---|
| ANISOU | 1675 | NH1 | ARG A | 606 | 4845 | 4766 | 4756 | 88 | −69 | 72 | N |

| | | | | | | | | | | |
|---|---|---|---|---|---|---|---|---|---|---|
| ATOM | 1676 | NH2 | ARG A | 606 | 38.655 | 26.909 | 88.513 | 1.00 | 40.36 | N |

| | | | | | | | | | | | |
|---|---|---|---|---|---|---|---|---|---|---|---|
| ANISOU | 1676 | NH2 | ARG A | 606 | 5115 | 5185 | 5034 | 24 | −103 | −129 | N |

| | | | | | | | | | | |
|---|---|---|---|---|---|---|---|---|---|---|
| ATOM | 1677 | N | MET A | 607 | 33.698 | 26.515 | 81.636 | 1.00 | 18.65 | N |

| | | | | | | | | | | | |
|---|---|---|---|---|---|---|---|---|---|---|---|
| ANISOU | 1677 | N | MET A | 607 | 2407 | 2017 | 2662 | −87 | −177 | 122 | N |

| | | | | | | | | | | |
|---|---|---|---|---|---|---|---|---|---|---|
| ATOM | 1678 | CA | MET A | 607 | 32.727 | 25.913 | 80.700 | 1.00 | 17.45 | C |

| | | | | | | | | | | | |
|---|---|---|---|---|---|---|---|---|---|---|---|
| ANISOU | 1678 | CA | MET A | 607 | 2118 | 2046 | 2467 | 66 | −29 | −52 | C |

| | | | | | | | | | | |
|---|---|---|---|---|---|---|---|---|---|---|
| ATOM | 1679 | C | MET A | 607 | 32.860 | 26.493 | 79.316 | 1.00 | 17.15 | C |

| | | | | | | | | | | | |
|---|---|---|---|---|---|---|---|---|---|---|---|
| ANISOU | 1679 | C | MET A | 607 | 2198 | 1824 | 2492 | 20 | −17 | −29 | C |

| | | | | | | | | | | |
|---|---|---|---|---|---|---|---|---|---|---|
| ATOM | 1680 | O | MET A | 607 | 32.678 | 25.805 | 78.280 | 1.00 | 17.55 | O |

| | | | | | | | | | | | |
|---|---|---|---|---|---|---|---|---|---|---|---|
| ANISOU | 1680 | O | MET A | 607 | 2300 | 1799 | 2569 | 195 | −146 | 14 | O |

| | | | | | | | | | | |
|---|---|---|---|---|---|---|---|---|---|---|
| ATOM | 1681 | CB | MET A | 607 | 31.296 | 26.054 | 81.261 | 1.00 | 17.74 | C |

| | | | | | | | | | | | |
|---|---|---|---|---|---|---|---|---|---|---|---|
| ANISOU | 1681 | CB | MET A | 607 | 2194 | 2041 | 2506 | 17 | −12 | −50 | C |

| | | | | | | | | | | |
|---|---|---|---|---|---|---|---|---|---|---|
| ATOM | 1682 | CG | MET A | 607 | 30.294 | 25.283 | 80.411 | 1.00 | 18.24 | C |

| | | | | | | | | | | | |
|---|---|---|---|---|---|---|---|---|---|---|---|
| ANISOU | 1682 | CG | MET A | 607 | 2330 | 1868 | 2732 | 14 | −22 | −86 | C |

| | | | | | | | | | | |
|---|---|---|---|---|---|---|---|---|---|---|
| ATOM | 1683 | SD | MET A | 607 | 30.433 | 23.522 | 80.870 | 1.00 | 21.00 | S |

| | | | | | | | | | | | |
|---|---|---|---|---|---|---|---|---|---|---|---|
| ANISOU | 1683 | SD | MET A | 607 | 2754 | 1772 | 3455 | 20 | 274 | 11 | S |

| | | | | | | | | | | |
|---|---|---|---|---|---|---|---|---|---|---|
| ATOM | 1684 | CE | MET A | 607 | 28.922 | 22.888 | 80.130 | 1.00 | 23.17 | C |

| | | | | | | | | | | | |
|---|---|---|---|---|---|---|---|---|---|---|---|
| ANISOU | 1684 | CE | MET A | 607 | 3229 | 2264 | 3312 | −110 | −4 | −70 | C |

| | | | | | | | | | | |
|---|---|---|---|---|---|---|---|---|---|---|
| ATOM | 1685 | N | ARG A | 608 | 33.029 | 27.838 | 79.234 | 1.00 | 16.78 | N |

| | | | | | | | | | | | |
|---|---|---|---|---|---|---|---|---|---|---|---|
| ANISOU | 1685 | N | ARG A | 608 | 2217 | 1769 | 2391 | −179 | −19 | 49 | N |

| | | | | | | | | | | |
|---|---|---|---|---|---|---|---|---|---|---|
| ATOM | 1686 | CA | ARG A | 608 | 33.181 | 28.429 | 77.908 | 1.00 | 17.63 | C |

| | | | | | | | | | | | |
|---|---|---|---|---|---|---|---|---|---|---|---|
| ANISOU | 1686 | CA | ARG A | 608 | 2363 | 1974 | 2360 | 30 | 30 | −43 | C |

| | | | | | | | | | | |
|---|---|---|---|---|---|---|---|---|---|---|
| ATOM | 1687 | C | ARG A | 608 | 34.321 | 27.799 | 77.119 | 1.00 | 17.62 | C |

| | | | | | | | | | | | |
|---|---|---|---|---|---|---|---|---|---|---|---|
| ANISOU | 1687 | C | ARG A | 608 | 2356 | 1929 | 2408 | −79 | −55 | 61 | C |

| | | | | | | | | | | |
|---|---|---|---|---|---|---|---|---|---|---|
| ATOM | 1688 | O | ARG A | 608 | 34.190 | 27.502 | 75.925 | 1.00 | 17.32 | O |

| | | | | | | | | | | | |
|---|---|---|---|---|---|---|---|---|---|---|---|
| ANISOU | 1688 | O | ARG A | 608 | 2299 | 1917 | 2366 | 252 | 3 | 66 | O |

| | | | | | | | | | | |
|---|---|---|---|---|---|---|---|---|---|---|
| ATOM | 1689 | CB | ARG A | 608 | 33.449 | 29.942 | 77.991 | 1.00 | 17.88 | C |

| | | | | | | | | | | | |
|---|---|---|---|---|---|---|---|---|---|---|---|
| ANISOU | 1689 | CB | ARG A | 608 | 2112 | 2023 | 2658 | −163 | 54 | −138 | C |

| | | | | | | | | | | |
|---|---|---|---|---|---|---|---|---|---|---|
| ATOM | 1690 | CG | ARG A | 608 | 32.229 | 30.739 | 78.460 | 1.00 | 18.98 | C |

| | | | | | | | | | | | |
|---|---|---|---|---|---|---|---|---|---|---|---|
| ANISOU | 1690 | CG | ARG A | 608 | 2551 | 1958 | 2703 | 128 | −14 | 11 | C |

| | | | | | | | | | | |
|---|---|---|---|---|---|---|---|---|---|---|
| ATOM | 1691 | CD | ARG A | 608 | 32.666 | 32.253 | 78.576 | 1.00 | 17.55 | C |

| | | | | | | | | | | | |
|---|---|---|---|---|---|---|---|---|---|---|---|
| ANISOU | 1691 | CD | ARG A | 608 | 2249 | 1946 | 2474 | −71 | 193 | −106 | C |

| | | | | | | | | | | |
|---|---|---|---|---|---|---|---|---|---|---|
| ATOM | 1692 | NE | ARG A | 608 | 31.394 | 32.915 | 78.980 | 1.00 | 17.14 | N |

| | | | | | | | | | | | |
|---|---|---|---|---|---|---|---|---|---|---|---|
| ANISOU | 1692 | NE | ARG A | 608 | 2186 | 1846 | 2482 | 97 | −144 | −33 | N |

| | | | | | | | | | | |
|---|---|---|---|---|---|---|---|---|---|---|
| ATOM | 1693 | CZ | ARG A | 608 | 30.504 | 33.396 | 78.114 | 1.00 | 15.88 | C |

| | | | | | | | | | | | |
|---|---|---|---|---|---|---|---|---|---|---|---|
| ANISOU | 1693 | CZ | ARG A | 608 | 2051 | 1640 | 2343 | 97 | 109 | 27 | C |

| | | | | | | | | | | |
|---|---|---|---|---|---|---|---|---|---|---|
| ATOM | 1694 | NH1 | ARG A | 608 | 30.772 | 33.443 | 76.823 | 1.00 | 15.44 | N |

| | | | | | | | | | | | |
|---|---|---|---|---|---|---|---|---|---|---|---|
| ANISOU | 1694 | NH1 | ARG A | 608 | 2086 | 1450 | 2331 | −66 | 77 | 64 | N |

| | | | | | | | | | | |
|---|---|---|---|---|---|---|---|---|---|---|
| ATOM | 1695 | NH2 | ARG A | 608 | 29.340 | 33.848 | 78.621 | 1.00 | 16.41 | N |

| | | | | | | | | | | | |
|---|---|---|---|---|---|---|---|---|---|---|---|
| ANISOU | 1695 | NH2 | ARG A | 608 | 1942 | 1619 | 2673 | 98 | 179 | −61 | N |

| | | | | | | | | | | |
|---|---|---|---|---|---|---|---|---|---|---|
| ATOM | 1696 | N | ARG A | 609 | 35.453 | 27.620 | 77.789 | 1.00 | 18.06 | N |

| | | | | | | | | | | | |
|---|---|---|---|---|---|---|---|---|---|---|---|
| ANISOU | 1696 | N | ARG A | 609 | 2279 | 2080 | 2503 | −8 | 18 | −48 | N |

| | | | | | | | | | | |
|---|---|---|---|---|---|---|---|---|---|---|
| ATOM | 1697 | CA | ARG A | 609 | 36.611 | 27.001 | 77.142 | 1.00 | 17.41 | C |

| | | | | | | | | | | | |
|---|---|---|---|---|---|---|---|---|---|---|---|
| ANISOU | 1697 | CA | ARG A | 609 | 2022 | 2049 | 2543 | 13 | 56 | 1 | C |

| | | | | | | | | | | |
|---|---|---|---|---|---|---|---|---|---|---|
| ATOM | 1698 | C | ARG A | 609 | 36.359 | 25.524 | 76.856 | 1.00 | 17.72 | C |

| | | | | | | | | | | | |
|---|---|---|---|---|---|---|---|---|---|---|---|
| ANISOU | 1698 | C | ARG A | 609 | 2243 | 2089 | 2400 | −52 | 49 | 16 | C |

| | | | | | | | | | | |
|---|---|---|---|---|---|---|---|---|---|---|
| ATOM | 1699 | O | ARG A | 609 | 36.785 | 25.068 | 75.798 | 1.00 | 17.83 | O |

| | | | | | | | | | | | |
|---|---|---|---|---|---|---|---|---|---|---|---|
| ANISOU | 1699 | O | ARG A | 609 | 2305 | 1896 | 2575 | −72 | 73 | −206 | O |

| | | | | | | | | | | |
|---|---|---|---|---|---|---|---|---|---|---|
| ATOM | 1700 | CB | ARG A | 609 | 37.832 | 27.155 | 78.056 | 1.00 | 18.95 | C |

| | | | | | | | | | | | |
|---|---|---|---|---|---|---|---|---|---|---|---|
| ANISOU | 1700 | CB | ARG A | 609 | 2357 | 2325 | 2517 | 51 | −52 | 31 | C |

| | | | | | | | | | | |
|---|---|---|---|---|---|---|---|---|---|---|
| ATOM | 1701 | CG | ARG A | 609 | 38.083 | 28.575 | 78.532 | 1.00 | 21.70 | C |

| | | | | | | | | | | | |
|---|---|---|---|---|---|---|---|---|---|---|---|
| ANISOU | 1701 | CG | ARG A | 609 | 2856 | 2580 | 2809 | −112 | 68 | −75 | C |

| | | | | | | | | | | |
|---|---|---|---|---|---|---|---|---|---|---|
| ATOM | 1702 | CD | ARG A | 609 | 38.122 | 29.614 | 77.417 | 1.00 | 21.87 | C |

| | | | | | | | | | | | |
|---|---|---|---|---|---|---|---|---|---|---|---|
| ANISOU | 1702 | CD | ARG A | 609 | 2675 | 2615 | 3020 | 106 | 13 | 73 | C |

| | | | | | | | | | | |
|---|---|---|---|---|---|---|---|---|---|---|
| ATOM | 1703 | NE | ARG A | 609 | 39.401 | 29.723 | 76.724 | 1.00 | 24.82 | N |

| | | | | | | | | | | | |
|---|---|---|---|---|---|---|---|---|---|---|---|
| ANISOU | 1703 | NE | ARG A | 609 | 2951 | 3140 | 3338 | −90 | 75 | −19 | N |

| | | | | | | | | | | |
|---|---|---|---|---|---|---|---|---|---|---|
| ATOM | 1704 | CZ | ARG A | 609 | 39.663 | 30.813 | 75.980 | 1.00 | 25.69 | C |

| | | | | | | | | | | | |
|---|---|---|---|---|---|---|---|---|---|---|---|
| ANISOU | 1704 | CZ | ARG A | 609 | 3153 | 3218 | 3391 | 19 | 52 | 56 | C |

| | | | | | | | | | | |
|---|---|---|---|---|---|---|---|---|---|---|
| ATOM | 1705 | NH1 | ARG A | 609 | 40.843 | 30.889 | 75.381 | 1.00 | 26.94 | N |

| | | | | | | | | | | | |
|---|---|---|---|---|---|---|---|---|---|---|---|
| ANISOU | 1705 | NH1 | ARG A | 609 | 3289 | 3403 | 3543 | −132 | 194 | −78 | N |

| | | | | | | | | | | |
|---|---|---|---|---|---|---|---|---|---|---|
| ATOM | 1706 | NH2 | ARG A | 609 | 38.761 | 31.798 | 75.850 | 1.00 | 25.09 | N |

| | | | | | | | | | | | |
|---|---|---|---|---|---|---|---|---|---|---|---|
| ANISOU | 1706 | NH2 | ARG A | 609 | 3025 | 3077 | 3430 | −96 | 172 | 64 | N |

| | | | | | | | | | | |
|---|---|---|---|---|---|---|---|---|---|---|
| ATOM | 1707 | N | PHE A | 610 | 35.615 | 24.839 | 77.721 | 1.00 | 17.97 | N |

| | | | | | | | | | | | |
|---|---|---|---|---|---|---|---|---|---|---|---|
| ANISOU | 1707 | N | PHE A | 610 | 2169 | 2177 | 2461 | −42 | 71 | 116 | N |

| | | | | | | | | | | |
|---|---|---|---|---|---|---|---|---|---|---|
| ATOM | 1708 | CA | PHE A | 610 | 35.353 | 23.406 | 77.523 | 1.00 | 17.48 | C |

| | | | | | | | | | | | |
|---|---|---|---|---|---|---|---|---|---|---|---|
| ANISOU | 1708 | CA | PHE A | 610 | 2024 | 2123 | 2495 | 162 | 75 | 21 | C |

| | | | | | | | | | | |
|---|---|---|---|---|---|---|---|---|---|---|
| ATOM | 1709 | C | PHE A | 610 | 34.494 | 23.220 | 76.298 | 1.00 | 17.42 | C |

| | | | | | | | | | | | |
|---|---|---|---|---|---|---|---|---|---|---|---|
| ANISOU | 1709 | C | PHE A | 610 | 2114 | 2179 | 2325 | 66 | 201 | −2 | C |

| | | | | | | | | | | |
|---|---|---|---|---|---|---|---|---|---|---|
| ATOM | 1710 | O | PHE A | 610 | 34.816 | 22.420 | 75.417 | 1.00 | 18.23 | O |

| | | | | | | | | | | | |
|---|---|---|---|---|---|---|---|---|---|---|---|
| ANISOU | 1710 | O | PHE A | 610 | 2474 | 2083 | 2370 | 229 | 142 | 65 | O |

| | | | | | | | | | | |
|---|---|---|---|---|---|---|---|---|---|---|
| ATOM | 1711 | CB | PHE A | 610 | 34.683 | 22.863 | 78.783 | 1.00 | 18.11 | C |

| | | | | | | | | | | | |
|---|---|---|---|---|---|---|---|---|---|---|---|
| ANISOU | 1711 | CB | PHE A | 610 | 2222 | 2089 | 2570 | 217 | 122 | 94 | C |

| | | | | | | | | | | |
|---|---|---|---|---|---|---|---|---|---|---|
| ATOM | 1712 | CG | PHE A | 610 | 34.227 | 21.417 | 78.689 | 1.00 | 18.67 | C |

| | | | | | | | | | | | |
|---|---|---|---|---|---|---|---|---|---|---|---|
| ANISOU | 1712 | CG | PHE A | 610 | 2564 | 2220 | 2310 | −39 | 55 | 219 | C |

| | | | | | | | | | | |
|---|---|---|---|---|---|---|---|---|---|---|
| ATOM | 1713 | CD1 | PHE A | 610 | 35.118 | 20.454 | 78.281 | 1.00 | 20.71 | C |

| | | | | | | | | | | | |
|---|---|---|---|---|---|---|---|---|---|---|---|
| ANISOU | 1713 | CDl | PHE A | 610 | 2821 | 2309 | 2737 | 98 | 24 | 157 | C |

| | | | | | | | | | | |
|---|---|---|---|---|---|---|---|---|---|---|
| ATOM | 1714 | CD2 | PHE A | 610 | 32.915 | 21.063 | 79.003 | 1.00 | 21.16 | C |

| | | | | | | | | | | | |
|---|---|---|---|---|---|---|---|---|---|---|---|
| ANISOU | 1714 | CD2 | PHE A | 610 | 2602 | 2602 | 2834 | −148 | 56 | 104 | C |

| | | | | | | | | | | |
|---|---|---|---|---|---|---|---|---|---|---|
| ATOM | 1715 | CE1 | PHE A | 610 | 34.719 | 19.116 | 78.219 | 1.00 | 21.69 | C |

| | | | | | | | | | | | |
|---|---|---|---|---|---|---|---|---|---|---|---|
| ANISOU | 1715 | CE1 | PHE A | 610 | 2966 | 2382 | 2894 | 32 | −56 | 122 | C |

| | | | | | | | | | | |
|---|---|---|---|---|---|---|---|---|---|---|
| ATOM | 1716 | CE2 | PHE A | 610 | 32.547 | 19.718 | 78.977 | 1.00 | 22.54 | C |

| | | | | | | | | | | | |
|---|---|---|---|---|---|---|---|---|---|---|---|
| ANISOU | 1716 | CE2 | PHE A | 610 | 3038 | 2531 | 2997 | −67 | 238 | 29 | C |

| | | | | | | | | | | |
|---|---|---|---|---|---|---|---|---|---|---|
| ATOM | 1717 | CZ | PHE A | 610 | 33.426 | 18.739 | 78.552 | 1.00 | 21.82 | C |

| | | | | | | | | | | | |
|---|---|---|---|---|---|---|---|---|---|---|---|
| ANISOU | 1717 | CZ | PHE A | 610 | 2836 | 2503 | 2951 | 73 | 43 | 209 | C |

| | | | | | | | | | | |
|---|---|---|---|---|---|---|---|---|---|---|
| ATOM | 1718 | N | ILE A | 611 | 33.380 | 23.998 | 76.222 | 1.00 | 16.24 | N |

| | | | | | | | | | | | |
|---|---|---|---|---|---|---|---|---|---|---|---|
| ANISOU | 1718 | N | ILE A | 611 | 2012 | 1977 | 2182 | 127 | 16 | 228 | N |

| | | | | | | | | | | |
|---|---|---|---|---|---|---|---|---|---|---|
| ATOM | 1719 | CA | ILE A | 611 | 32.527 | 23.864 | 75.035 | 1.00 | 16.24 | C |

| | | | | | | | | | | | |
|---|---|---|---|---|---|---|---|---|---|---|---|
| ANISON | 1719 | CA | ILE A | 611 | 1939 | 1952 | 2280 | 201 | 12 | −37 | C |

| | | | | | | | | | | |
|---|---|---|---|---|---|---|---|---|---|---|
| ATOM | 1720 | C | ILE A | 611 | 33.310 | 24.212 | 73.785 | 1.00 | 16.58 | C |

| | | | | | | | | | | | |
|---|---|---|---|---|---|---|---|---|---|---|---|
| ANISOU | 1720 | C | ILE A | 611 | 2108 | 1858 | 2332 | 115 | 30 | −64 | C |

| | | | | | | | | | | |
|---|---|---|---|---|---|---|---|---|---|---|
| ATOM | 1721 | O | ILE A | 611 | 33.189 | 23.530 | 72.756 | 1.00 | 17.64 | O |

| | | | | | | | | | | | |
|---|---|---|---|---|---|---|---|---|---|---|---|
| ANISOU | 1721 | O | ILE A | 611 | 2382 | 2036 | 2285 | 181 | −4 | −69 | O |

| | | | | | | | | | | |
|---|---|---|---|---|---|---|---|---|---|---|
| ATOM | 1722 | CB | ILE A | 611 | 31.255 | 24.731 | 75.132 | 1.00 | 15.45 | C |

| | | | | | | | | | | | |
|---|---|---|---|---|---|---|---|---|---|---|---|
| ANISOU | 1722 | CB | ILE A | 611 | 1902 | 1920 | 2050 | 285 | 104 | −116 | C |

| | | | | | | | | | | |
|---|---|---|---|---|---|---|---|---|---|---|
| ATOM | 1723 | CG1 | ILE A | 611 | 30.431 | 24.331 | 76.354 | 1.00 | 16.00 | C |

| | | | | | | | | | | | |
|---|---|---|---|---|---|---|---|---|---|---|---|
| ANISOU | 1723 | CG1 | ILE A | 611 | 1929 | 2044 | 2105 | 247 | 56 | −62 | C |

| | | | | | | | | | | |
|---|---|---|---|---|---|---|---|---|---|---|
| ATOM | 1724 | CG2 | ILE A | 611 | 30.424 | 24.655 | 73.846 | 1.00 | 16.68 | C |

| | | | | | | | | | | | |
|---|---|---|---|---|---|---|---|---|---|---|---|
| ANISON | 1724 | CG2 | ILE A | 611 | 2073 | 2138 | 2127 | 41 | 70 | −19 | C |

| | | | | | | | | | | |
|---|---|---|---|---|---|---|---|---|---|---|
| ATOM | 1725 | CD1 | ILE A | 611 | 29.280 | 25.311 | 76.605 | 1.00 | 16.86 | C |

| | | | | | | | | | | | |
|---|---|---|---|---|---|---|---|---|---|---|---|
| ANISOU | 1725 | CD1 | ILE A | 611 | 2262 | 2219 | 1925 | 401 | −30 | −205 | C |

| | | | | | | | | | | |
|---|---|---|---|---|---|---|---|---|---|---|
| ATOM | 1726 | N | ALA A | 612 | 34.115 | 25.268 | 73.772 | 1.00 | 18.62 | N |

| | | | | | | | | | | | |
|---|---|---|---|---|---|---|---|---|---|---|---|
| ANISOU | 1726 | N | ALA A | 612 | 2225 | 2017 | 2834 | 152 | 170 | −39 | N |

| | | | | | | | | | | |
|---|---|---|---|---|---|---|---|---|---|---|
| ATOM | 1727 | CA | ALA A | 612 | 34.911 | 25.624 | 72.624 | 1.00 | 18.38 | C |

| | | | | | | | | | | | |
|---|---|---|---|---|---|---|---|---|---|---|---|
| ANISOU | 1727 | CA | ALA A | 612 | 2398 | 2081 | 2504 | 55 | 68 | −93 | C |

| | | | | | | | | | | |
|---|---|---|---|---|---|---|---|---|---|---|
| ATOM | 1728 | C | ALA A | 612 | 35.832 | 24.451 | 72.239 | 1.00 | 19.28 | C |

| | | | | | | | | | | | |
|---|---|---|---|---|---|---|---|---|---|---|---|
| ANISOU | 1728 | C | ALA A | 612 | 2371 | 2340 | 2613 | 185 | 70 | −14 | C |

| | | | | | | | | | | |
|---|---|---|---|---|---|---|---|---|---|---|
| ATOM | 1729 | O | ALA A | 612 | 35.929 | 24.157 | 71.030 | 1.00 | 19.78 | O |

| | | | | | | | | | | | |
|---|---|---|---|---|---|---|---|---|---|---|---|
| ANISOU | 1729 | O | ALA A | 612 | 2372 | 2552 | 2594 | 143 | 312 | 52 | O |

| | | | | | | | | | | |
|---|---|---|---|---|---|---|---|---|---|---|
| ATOM | 1730 | CB | ALA A | 612 | 35.736 | 26.885 | 72.841 | 1.00 | 19.07 | C |

| | | | | | | | | | | | |
|---|---|---|---|---|---|---|---|---|---|---|---|
| ANISOU | 1730 | CB | ALA A | 612 | 2617 | 2230 | 2400 | −100 | 30 | −45 | C |

| | | | | | | | | | | |
|---|---|---|---|---|---|---|---|---|---|---|
| ATOM | 1731 | N | HIS A | 613 | 36.474 | 23.821 | 73.221 | 1.00 | 19.98 | N |

| | | | | | | | | | | | |
|---|---|---|---|---|---|---|---|---|---|---|---|
| ANISOU | 1731 | N | HIS A | 613 | 2309 | 2476 | 2806 | 124 | 9 | −62 | N |

| | | | | | | | | | | |
|---|---|---|---|---|---|---|---|---|---|---|
| ATOM | 1732 | CA | HIS A | 613 | 37.329 | 22.651 | 72.937 | 1.00 | 20.47 | C |

| | | | | | | | | | | | |
|---|---|---|---|---|---|---|---|---|---|---|---|
| ANISOU | 1732 | CA | HIS A | 613 | 2369 | 2469 | 2941 | 18 | 80 | 62 | C |

| | | | | | | | | | | |
|---|---|---|---|---|---|---|---|---|---|---|
| ATOM | 1733 | C | HIS A | 613 | 36.547 | 21.510 | 72.290 | 1.00 | 21.13 | C |

| | | | | | | | | | | | |
|---|---|---|---|---|---|---|---|---|---|---|---|
| ANISOU | 1733 | C | HIS A | 613 | 2664 | 2544 | 2821 | −1 | −62 | −23 | C |

| | | | | | | | | | | |
|---|---|---|---|---|---|---|---|---|---|---|
| ATOM | 1734 | O | HIS A | 613 | 37.060 | 20.814 | 71.381 | 1.00 | 21.74 | O |

| | | | | | | | | | | | |
|---|---|---|---|---|---|---|---|---|---|---|---|
| ANISOU | 1734 | O | HIS A | 613 | 2330 | 2938 | 2991 | 14 | 83 | 17 | O |

| | | | | | | | | | | |
|---|---|---|---|---|---|---|---|---|---|---|
| ATOM | 1735 | CB | HIS A | 613 | 37.903 | 22.187 | 74.295 | 1.00 | 22.31 | C |

| | | | | | | | | | | | |
|---|---|---|---|---|---|---|---|---|---|---|---|
| ANISOU | 1735 | CB | HIS A | 613 | 2631 | 2841 | 3003 | 47 | 14 | −116 | C |

| | | | | | | | | | | |
|---|---|---|---|---|---|---|---|---|---|---|
| ATOM | 1736 | CG | HIS A | 613 | 38.624 | 20.871 | 74.229 | 1.00 | 24.90 | C |

| | | | | | | | | | | | |
|---|---|---|---|---|---|---|---|---|---|---|---|
| ANISON | 1736 | CG | HIS A | 613 | 3020 | 3051 | 3390 | 203 | −67 | −22 | C |

| | | | | | | | | | | |
|---|---|---|---|---|---|---|---|---|---|---|
| ATOM | 1737 | ND1 | HIS A | 613 | 39.879 | 20.744 | 73.679 | 1.00 | 27.12 | N |

| | | | | | | | | | | | |
|---|---|---|---|---|---|---|---|---|---|---|---|
| ANISOU | 1737 | ND1 | HIS A | 613 | 3270 | 3437 | 3598 | 175 | 104 | −32 | N |

| | | | | | | | | | | |
|---|---|---|---|---|---|---|---|---|---|---|
| ATOM | 1738 | CD2 | HIS A | 613 | 38.217 | 19.650 | 74.631 | 1.00 | 26.89 | C |

| | | | | | | | | | | | |
|---|---|---|---|---|---|---|---|---|---|---|---|
| ANISOU | 1738 | CD2 | HIS A | 613 | 3397 | 3212 | 3608 | 156 | −67 | 101 | C |

| | | | | | | | | | | |
|---|---|---|---|---|---|---|---|---|---|---|
| ATOM | 1739 | CE1 | HIS A | 613 | 40.233 | 19.464 | 73.770 | 1.00 | 27.29 | C |

| | | | | | | | | | | | |
|---|---|---|---|---|---|---|---|---|---|---|---|
| ANISOU | 1739 | CE1 | HIS A | 613 | 3385 | 3415 | 3569 | 128 | −5 | −39 | C |

| | | | | | | | | | | |
|---|---|---|---|---|---|---|---|---|---|---|
| ATOM | 1740 | NE2 | HIS A | 613 | 39.255 | 18.777 | 74.316 | 1.00 | 28.03 | N |

| | | | | | | | | | | | |
|---|---|---|---|---|---|---|---|---|---|---|---|
| ANISOU | 1740 | NE2 | HIS A | 613 | 3602 | 3425 | 3622 | 209 | 134 | 33 | N |

| | | | | | | | | | | |
|---|---|---|---|---|---|---|---|---|---|---|
| ATOM | 1741 | N | LEU A | 614 | 35.327 | 21.253 | 72.788 | 1.00 | 19.66 | N |

| | | | | | | | | | | | |
|---|---|---|---|---|---|---|---|---|---|---|---|
| ANISOU | 1741 | N | LEU A | 614 | 2392 | 2269 | 2811 | 80 | −163 | 19 | N |

| | | | | | | | | | | |
|---|---|---|---|---|---|---|---|---|---|---|
| ATOM | 1742 | CA | LEU A | 614 | 34.491 | 20.168 | 72.213 | 1.00 | 20.01 | C |

| | | | | | | | | | | | |
|---|---|---|---|---|---|---|---|---|---|---|---|
| ANISOU | 1742 | CA | LEU A | 614 | 2508 | 2301 | 2793 | 35 | 128 | −122 | C |

| | | | | | | | | | | |
|---|---|---|---|---|---|---|---|---|---|---|
| ATOM | 1743 | C | LEU A | 614 | 34.231 | 20.439 | 70.750 | 1.00 | 19.65 | C |

| | | | | | | | | | | | |
|---|---|---|---|---|---|---|---|---|---|---|---|
| ANISOU | 1743 | C | LEU A | 614 | 2353 | 2291 | 2821 | 107 | 89 | −76 | C |

| | | | | | | | | | | |
|---|---|---|---|---|---|---|---|---|---|---|
| ATOM | 1744 | O | LEU A | 614 | 34.239 | 19.536 | 69.893 | 1.00 | 22.24 | O |

| | | | | | | | | | | | |
|---|---|---|---|---|---|---|---|---|---|---|---|
| ANISOU | 1744 | O | LEU A | 614 | 2787 | 2573 | 3090 | 197 | 110 | −310 | O |

| | | | | | | | | | | |
|---|---|---|---|---|---|---|---|---|---|---|
| ATOM | 1745 | CB | LEU A | 614 | 33.169 | 19.989 | 72.985 | 1.00 | 19.74 | C |

| | | | | | | | | | | | |
|---|---|---|---|---|---|---|---|---|---|---|---|
| ANISOU | 1745 | CB | LEU A | 614 | 2497 | 2272 | 2730 | 44 | 63 | −50 | C |

| | | | | | | | | | | |
|---|---|---|---|---|---|---|---|---|---|---|
| ATOM | 1746 | CG | LEU A | 614 | 33.308 | 19.558 | 74.459 | 1.00 | 19.67 | C |

| | | | | | | | | | | | |
|---|---|---|---|---|---|---|---|---|---|---|---|
| ANISOU | 1746 | CG | LEU A | 614 | 2396 | 2295 | 2784 | 215 | 83 | −35 | C |

| | | | | | | | | | | |
|---|---|---|---|---|---|---|---|---|---|---|
| ATOM | 1747 | CD1 | LEU A | 614 | 31.949 | 19.510 | 75.146 | 1.00 | 19.67 | C |

| | | | | | | | | | | | |
|---|---|---|---|---|---|---|---|---|---|---|---|
| ANISOU | 1747 | CD1 | LEU A | 614 | 2708 | 2145 | 2621 | −57 | 207 | 10 | C |

| | | | | | | | | | | |
|---|---|---|---|---|---|---|---|---|---|---|
| ATOM | 1748 | CD2 | LEU A | 614 | 33.944 | 18.152 | 74.506 | 1.00 | 20.94 | C |

| | | | | | | | | | | | |
|---|---|---|---|---|---|---|---|---|---|---|---|
| ANISOU | 1748 | CD2 | LEU A | 614 | 3022 | 2012 | 2923 | 15 | 120 | 40 | C |

| | | | | | | | | | | |
|---|---|---|---|---|---|---|---|---|---|---|
| ATOM | 1749 | N | ILE A | 615 | 33.965 | 21.698 | 70.391 | 1.00 | 18.12 | N |

| | | | | | | | | | | | |
|---|---|---|---|---|---|---|---|---|---|---|---|
| ANISOU | 1749 | N | ILE A | 615 | 2168 | 2250 | 2465 | −32 | 144 | 31 | N |

| | | | | | | | | | | |
|---|---|---|---|---|---|---|---|---|---|---|
| ATOM | 1750 | CA | ILE A | 615 | 33.720 | 22.059 | 68.995 | 1.00 | 18.12 | C |

| | | | | | | | | | | | |
|---|---|---|---|---|---|---|---|---|---|---|---|
| ANISOU | 1750 | CA | ILE A | 615 | 2398 | 2077 | 2409 | 99 | 85 | −72 | C |

| | | | | | | | | | | |
|---|---|---|---|---|---|---|---|---|---|---|
| ATOM | 1751 | C | ILE A | 615 | 34.967 | 21.831 | 68.148 | 1.00 | 20.48 | C |

| | | | | | | | | | | | |
|---|---|---|---|---|---|---|---|---|---|---|---|
| ANISOU | 1751 | C | ILE A | 615 | 2572 | 2621 | 2588 | −51 | 178 | −32 | C |

| | | | | | | | | | | |
|---|---|---|---|---|---|---|---|---|---|---|
| ATOM | 1752 | O | ILE A | 615 | 34.860 | 21.203 | 67.080 | 1.00 | 22.69 | O |

| | | | | | | | | | | | |
|---|---|---|---|---|---|---|---|---|---|---|---|
| ANISOU | 1752 | O | ILE A | 615 | 2927 | 2694 | 3001 | −15 | 307 | −278 | O |

| | | | | | | | | | | |
|---|---|---|---|---|---|---|---|---|---|---|
| ATOM | 1753 | CB | ILE A | 615 | 33.263 | 23.546 | 68.925 | 1.00 | 18.30 | C |

| | | | | | | | | | | | |
|---|---|---|---|---|---|---|---|---|---|---|---|
| ANISOU | 1753 | CB | ILE A | 615 | 2496 | 2017 | 2441 | 25 | 86 | −122 | C |

| | | | | | | | | | | |
|---|---|---|---|---|---|---|---|---|---|---|
| ATOM | 1754 | CG1 | ILE A | 615 | 31.880 | 23.653 | 69.547 | 1.00 | 17.52 | C |

| | | | | | | | | | | | |
|---|---|---|---|---|---|---|---|---|---|---|---|
| ANISOU | 1754 | CG1 | ILE A | 615 | 2422 | 1987 | 2248 | −41 | 134 | −141 | C |

| | | | | | | | | | | |
|---|---|---|---|---|---|---|---|---|---|---|
| ATOM | 1755 | CG2 | ILE A | 615 | 33.228 | 23.975 | 67.481 | 1.00 | 20.24 | C |

| | | | | | | | | | | | |
|---|---|---|---|---|---|---|---|---|---|---|---|
| ANISOU | 1755 | CG2 | ILE A | 615 | 2754 | 2517 | 2418 | 88 | 45 | −112 | C |

| | | | | | | | | | | |
|---|---|---|---|---|---|---|---|---|---|---|
| ATOM | 1756 | CD1 | ILE A | 615 | 31.356 | 25.064 | 69.831 | 1.00 | 18.55 | C |

| | | | | | | | | | | | |
|---|---|---|---|---|---|---|---|---|---|---|---|
| ANISOU | 1756 | CD1 | ILE A | 615 | 2472 | 2053 | 2523 | 56 | 20 | −102 | C |

| | | | | | | | | | | |
|---|---|---|---|---|---|---|---|---|---|---|
| ATOM | 1757 | N | LEU A | 616 | 36.102 | 22.327 | 68.598 | 1.00 | 21.45 | N |

| | | | | | | | | | | | |
|---|---|---|---|---|---|---|---|---|---|---|---|
| ANISOU | 1757 | N | LEU A | 616 | 2581 | 2872 | 2696 | −49 | 166 | −217 | N |

| | | | | | | | | | | |
|---|---|---|---|---|---|---|---|---|---|---|
| ATOM | 1758 | CA | LEU A | 616 | 37.342 | 22.218 | 67.795 | 1.00 | 22.75 | C |

| | | | | | | | | | | | |
|---|---|---|---|---|---|---|---|---|---|---|---|
| ANISOU | 1758 | CA | LEU A | 616 | 2616 | 3016 | 3014 | 95 | 242 | −179 | C |

| | | | | | | | | | | |
|---|---|---|---|---|---|---|---|---|---|---|
| ATOM | 1759 | C | LEU A | 616 | 37.803 | 20.779 | 67.676 | 1.00 | 24.30 | C |

| | | | | | | | | | | | |
|---|---|---|---|---|---|---|---|---|---|---|---|
| ANISOU | 1759 | C | LEU A | 616 | 3075 | 3016 | 3144 | 87 | 131 | −173 | C |

| | | | | | | | | | | |
|---|---|---|---|---|---|---|---|---|---|---|
| ATOM | 1760 | O | LEU A | 616 | 38.525 | 20.439 | 66.709 | 1.00 | 27.12 | O |

| | | | | | | | | | | | |
|---|---|---|---|---|---|---|---|---|---|---|---|
| ANISOU | 1760 | O | LEU A | 616 | 3319 | 3620 | 3367 | 101 | 421 | −246 | O |

| | | | | | | | | | | |
|---|---|---|---|---|---|---|---|---|---|---|
| ATOM | 1761 | CB | LEU A | 616 | 38.463 | 23.090 | 68.401 | 1.00 | 24.20 | C |

| | | | | | | | | | | | |
|---|---|---|---|---|---|---|---|---|---|---|---|
| ANISOU | 1761 | CB | LEU A | 616 | 2926 | 3134 | 3137 | 24 | 79 | −87 | C |

| | | | | | | | | | | |
|---|---|---|---|---|---|---|---|---|---|---|
| ATOM | 1762 | CG | LEU A | 616 | 38.143 | 24.601 | 68.424 | 1.00 | 23.96 | C |

| | | | | | | | | | | | |
|---|---|---|---|---|---|---|---|---|---|---|---|
| ANISOU | 1762 | CG | LEU A | 616 | 2932 | 3094 | 3077 | −67 | 43 | −97 | C |

| | | | | | | | | | | |
|---|---|---|---|---|---|---|---|---|---|---|
| ATOM | 1763 | CD1 | LEU A | 616 | 39.340 | 25.345 | 69.003 | 1.00 | 24.71 | C |

| | | | | | | | | | | | |
|---|---|---|---|---|---|---|---|---|---|---|---|
| ANISOU | 1763 | CD1 | LEU A | 616 | 2827 | 3304 | 3257 | −99 | 143 | −95 | C |

| | | | | | | | | | | |
|---|---|---|---|---|---|---|---|---|---|---|
| ATOM | 1764 | CD2 | LEU A | 616 | 37.687 | 25.127 | 67.070 | 1.00 | 24.18 | C |

| | | | | | | | | | | | |
|---|---|---|---|---|---|---|---|---|---|---|---|
| ANISOU | 1764 | CD2 | LEU A | 616 | 2823 | 3353 | 3010 | −112 | 183 | −93 | C |

| | | | | | | | | | | |
|---|---|---|---|---|---|---|---|---|---|---|
| ATOM | 1765 | N | THR A | 617 | 37.465 | 19.938 | 68.624 | 1.00 | 24.70 | N |

| | | | | | | | | | | | |
|---|---|---|---|---|---|---|---|---|---|---|---|
| ANISOU | 1765 | N | THR A | 617 | 3019 | 3206 | 3160 | 143 | 74 | −76 | N |

| | | | | | | | | | | |
|---|---|---|---|---|---|---|---|---|---|---|
| ATOM | 1766 | CA | THR A | 617 | 37.874 | 18.535 | 68.575 | 1.00 | 26.86 | C |

| | | | | | | | | | | | |
|---|---|---|---|---|---|---|---|---|---|---|---|
| ANISOU | 1766 | CA | THR A | 617 | 3444 | 3234 | 3529 | 140 | 6 | −81 | C |

| | | | | | | | | | | |
|---|---|---|---|---|---|---|---|---|---|---|
| ATOM | 1767 | C | THR A | 617 | 36.800 | 17.638 | 67.990 | 1.00 | 27.41 | C |

| | | | | | | | | | | | |
|---|---|---|---|---|---|---|---|---|---|---|---|
| ANISOU | 1767 | C | THR A | 617 | 3561 | 3318 | 3537 | 100 | −42 | −81 | C |

| | | | | | | | | | | |
|---|---|---|---|---|---|---|---|---|---|---|
| ATOM | 1768 | O | THR A | 617 | 36.922 | 16.401 | 68.061 | 1.00 | 27.99 | O |

| | | | | | | | | | | | |
|---|---|---|---|---|---|---|---|---|---|---|---|
| ANISOU | 1768 | O | THR A | 617 | 3683 | 3287 | 3663 | 205 | 22 | 49 | O |

| | | | | | | | | | | |
|---|---|---|---|---|---|---|---|---|---|---|
| ATOM | 1769 | CB | THR A | 617 | 38.363 | 18.063 | 69.958 | 1.00 | 27.91 | C |

| | | | | | | | | | | | |
|---|---|---|---|---|---|---|---|---|---|---|---|
| ANISOU | 1769 | CB | THR A | 617 | 3614 | 3416 | 3574 | 44 | 0 | 33 | C |

| | | | | | | | | | | |
|---|---|---|---|---|---|---|---|---|---|---|
| ATOM | 1770 | OG1 | THR A | 617 | 37.296 | 18.211 | 70.880 | 1.00 | 27.25 | O |

| | | | | | | | | | | | |
|---|---|---|---|---|---|---|---|---|---|---|---|
| ANISOU | 1770 | OG1 | THR A | 617 | 3519 | 3134 | 3702 | 273 | −46 | −40 | O |

| | | | | | | | | | | |
|---|---|---|---|---|---|---|---|---|---|---|
| ATOM | 1771 | CG2 | THR A | 617 | 39.576 | 18.891 | 70.379 | 1.00 | 28.06 | C |

| | | | | | | | | | | | |
|---|---|---|---|---|---|---|---|---|---|---|---|
| ANISOU | 1771 | CG2 | THR A | 617 | 3685 | 3282 | 3695 | 105 | −26 | −73 | C |

| | | | | | | | | | | |
|---|---|---|---|---|---|---|---|---|---|---|
| ATOM | 1772 | N | ASP A | 618 | 35.777 | 18.223 | 67.367 | 1.00 | 26.05 | N |

| | | | | | | | | | | | |
|---|---|---|---|---|---|---|---|---|---|---|---|
| ANISOU | 1772 | N | ASP A | 618 | 3386 | 3188 | 3323 | 184 | 145 | −162 | N |

| | | | | | | | | | | |
|---|---|---|---|---|---|---|---|---|---|---|
| ATOM | 1773 | CA | ASP A | 618 | 34.752 | 17.468 | 66.657 | 1.00 | 26.29 | C |

| | | | | | | | | | | | |
|---|---|---|---|---|---|---|---|---|---|---|---|
| ANISOU | 1773 | CA | ASP A | 618 | 3300 | 3247 | 3443 | 103 | 141 | −90 | C |

| | | | | | | | | | | |
|---|---|---|---|---|---|---|---|---|---|---|
| ATOM | 1774 | C | ASP A | 618 | 34.217 | 16.378 | 67.564 | 1.00 | 26.42 | C |

| | | | | | | | | | | | |
|---|---|---|---|---|---|---|---|---|---|---|---|
| ANISOU | 1774 | C | ASP A | 618 | 3335 | 3310 | 3394 | −1 | 36 | −48 | C |

| | | | | | | | | | | |
|---|---|---|---|---|---|---|---|---|---|---|
| ATOM | 1775 | O | ASP A | 618 | 34.106 | 15.184 | 67.231 | 1.00 | 28.47 | O |

| | | | | | | | | | | | |
|---|---|---|---|---|---|---|---|---|---|---|---|
| ANISOU | 1775 | O | ASP A | 618 | 3766 | 3429 | 3621 | 97 | 265 | −221 | O |

| | | | | | | | | | | |
|---|---|---|---|---|---|---|---|---|---|---|
| ATOM | 1776 | CB | ASP A | 618 | 35.387 | 16.865 | 65.402 | 1.00 | 27.42 | C |

| | | | | | | | | | | | |
|---|---|---|---|---|---|---|---|---|---|---|---|
| ANISOU | 1776 | CB | ASP A | 618 | 3593 | 3470 | 3355 | 18 | 124 | −86 | C |

| | | | | | | | | | | |
|---|---|---|---|---|---|---|---|---|---|---|
| ATOM | 1777 | CG | ASP A | 618 | 34.473 | 16.394 | 64.307 | 1.00 | 29.38 | C |

| | | | | | | | | | | | |
|---|---|---|---|---|---|---|---|---|---|---|---|
| ANISOU | 1777 | CG | ASP A | 618 | 3695 | 3827 | 3643 | 64 | 20 | −170 | C |

| | | | | | | | | | | |
|---|---|---|---|---|---|---|---|---|---|---|
| ATOM | 1778 | OD1 | ASP A | 618 | 33.272 | 16.690 | 64.259 | 1.00 | 28.30 | O |

| | | | | | | | | | | | |
|---|---|---|---|---|---|---|---|---|---|---|---|
| ANISOU | 1778 | OD1 | ASP A | 618 | 3575 | 3617 | 3561 | 46 | 30 | −177 | O |

| | | | | | | | | | | |
|---|---|---|---|---|---|---|---|---|---|---|
| ATOM | 1779 | OD2 | ASP A | 618 | 35.013 | 15.712 | 63.385 | 1.00 | 30.41 | O |

| | | | | | | | | | | | |
|---|---|---|---|---|---|---|---|---|---|---|---|
| ANISOU | 1779 | OD2 | ASP A | 618 | 3908 | 3839 | 3806 | 114 | 83 | −324 | O |

| | | | | | | | | | | |
|---|---|---|---|---|---|---|---|---|---|---|
| ATOM | 1780 | N | ALA A | 619 | 33.736 | 16.776 | 68.731 | 1.00 | 26.15 | N |

| | | | | | | | | | | | |
|---|---|---|---|---|---|---|---|---|---|---|---|
| ANISOU | 1780 | N | ALA A | 619 | 3280 | 3141 | 3515 | −6 | 105 | −129 | N |

| | | | | | | | | | | |
|---|---|---|---|---|---|---|---|---|---|---|
| ATOM | 1781 | CA | ALA A | 619 | 33.275 | 15.864 | 69.770 | 1.00 | 27.62 | C |

| | | | | | | | | | | | |
|---|---|---|---|---|---|---|---|---|---|---|---|
| ANISOU | 1781 | CA | ALA A | 619 | 3633 | 3383 | 3477 | 81 | 45 | −32 | C |

| | | | | | | | | | | |
|---|---|---|---|---|---|---|---|---|---|---|
| ATOM | 1782 | C | ALA A | 619 | 32.068 | 15.032 | 69.419 | 1.00 | 27.85 | C |

| | | | | | | | | | | | |
|---|---|---|---|---|---|---|---|---|---|---|---|
| ANISOU | 1782 | C | ALA A | 619 | 3762 | 3295 | 3526 | 73 | 63 | −134 | C |

| | | | | | | | | | | |
|---|---|---|---|---|---|---|---|---|---|---|
| ATOM | 1783 | O | ALA A | 619 | 31.819 | 14.056 | 70.136 | 1.00 | 28.62 | O |

| | | | | | | | | | | | |
|---|---|---|---|---|---|---|---|---|---|---|---|
| ANISOU | 1783 | O | ALA A | 619 | 4120 | 3361 | 3392 | −16 | 121 | −227 | O |

| | | | | | | | | | | |
|---|---|---|---|---|---|---|---|---|---|---|
| ATOM | 1784 | CB | ALA A | 619 | 33.022 | 16.646 | 71.066 | 1.00 | 29.09 | C |

| | | | | | | | | | | | |
|---|---|---|---|---|---|---|---|---|---|---|---|
| ANISOU | 1784 | CB | ALA A | 619 | 4019 | 3553 | 3483 | 52 | 91 | −75 | C |

| | | | | | | | | | | |
|---|---|---|---|---|---|---|---|---|---|---|
| ATOM | 1785 | N | LEU A | 620 | 31.326 | 15.357 | 68.371 | 1.00 | 26.99 | N |

| | | | | | | | | | | | |
|---|---|---|---|---|---|---|---|---|---|---|---|
| ANISOU | 1785 | N | LEU A | 620 | 3593 | 3232 | 3429 | 44 | 102 | −257 | N |

| | | | | | | | | | | |
|---|---|---|---|---|---|---|---|---|---|---|
| ATOM | 1786 | CA | LEU A | 620 | 30.151 | 14.556 | 68.024 | 1.00 | 27.73 | C |

| | | | | | | | | | | | |
|---|---|---|---|---|---|---|---|---|---|---|---|
| ANISOU | 1786 | CA | LEU A | 620 | 3608 | 3365 | 3564 | −6 | 36 | −209 | C |

| | | | | | | | | | | |
|---|---|---|---|---|---|---|---|---|---|---|
| ATOM | 1787 | C | LEU A | 620 | 30.529 | 13.416 | 67.070 | 1.00 | 30.37 | C |

| | | | | | | | | | | | |
|---|---|---|---|---|---|---|---|---|---|---|---|
| ANISOU | 1787 | C | LEU A | 620 | 4115 | 3680 | 3744 | 20 | 96 | −361 | C |

| | | | | | | | | | | |
|---|---|---|---|---|---|---|---|---|---|---|
| ATOM | 1788 | O | LEU A | 620 | 29.596 | 12.684 | 66.711 | 1.00 | 31.58 | O |

| | | | | | | | | | | | |
|---|---|---|---|---|---|---|---|---|---|---|---|
| ANISOU | 1788 | O | LEU A | 620 | 4099 | 3835 | 4064 | −36 | 218 | −362 | O |

| | | | | | | | | | | |
|---|---|---|---|---|---|---|---|---|---|---|
| ATOM | 1789 | CB | LEU A | 620 | 29.050 | 15.406 | 67.434 | 1.00 | 26.26 | C |

| | | | | | | | | | | | |
|---|---|---|---|---|---|---|---|---|---|---|---|
| ANISOU | 1789 | CB | LEU A | 620 | 3465 | 3153 | 3361 | −103 | 93 | −317 | C |

| | | | | | | | | | | |
|---|---|---|---|---|---|---|---|---|---|---|
| ATOM | 1790 | CG | LEU A | 620 | 28.679 | 16.652 | 68.271 | 1.00 | 25.84 | C |

| | | | | | | | | | | | |
|---|---|---|---|---|---|---|---|---|---|---|---|
| ANISOU | 1790 | CG | LEU A | 620 | 3365 | 3163 | 3290 | 44 | 50 | −245 | C |

| | | | | | | | | | | |
|---|---|---|---|---|---|---|---|---|---|---|
| ATOM | 1791 | CD1 | LEU A | 620 | 27.702 | 17.486 | 67.465 | 1.00 | 25.51 | C |

| | | | | | | | | | | | |
|---|---|---|---|---|---|---|---|---|---|---|---|
| ANISOU | 1791 | CD1 | LEU A | 620 | 3310 | 3188 | 3196 | −106 | 7 | −167 | C |

| | | | | | | | | | | |
|---|---|---|---|---|---|---|---|---|---|---|
| ATOM | 1792 | CD2 | LEU A | 620 | 28.148 | 16.243 | 69.630 | 1.00 | 26.24 | C |

| | | | | | | | | | | | |
|---|---|---|---|---|---|---|---|---|---|---|---|
| ANISOU | 1792 | CD2 | LEU A | 620 | 3379 | 3280 | 3310 | −85 | −37 | −155 | C |

| | | | | | | | | | | |
|---|---|---|---|---|---|---|---|---|---|---|
| ATOM | 1793 | N | LYS A | 621 | 31.796 | 13.208 | 66.768 | 1.00 | 32.72 | N |

| | | | | | | | | | | | |
|---|---|---|---|---|---|---|---|---|---|---|---|
| ANISOU | 1793 | N | LYS A | 621 | 4175 | 3990 | 4266 | 50 | 99 | −65 | N |

| | | | | | | | | | | |
|---|---|---|---|---|---|---|---|---|---|---|
| ATOM | 1794 | CA | LYS A | 621 | 32.184 | 12.071 | 65.922 | 1.00 | 36.62 | C |

| | | | | | | | | | | | |
|---|---|---|---|---|---|---|---|---|---|---|---|
| ANISOU | 1794 | CA | LYS A | 621 | 4758 | 4481 | 4676 | 149 | 201 | −311 | C |

| | | | | | | | | | | |
|---|---|---|---|---|---|---|---|---|---|---|
| ATOM | 1795 | C | LYS A | 621 | 31.137 | 11.666 | 64.901 | 1.00 | 38.99 | C |

| | | | | | | | | | | | |
|---|---|---|---|---|---|---|---|---|---|---|---|
| ANISOU | 1795 | C | LYS A | 621 | 4958 | 4897 | 4960 | 54 | 19 | −146 | C |

| | | | | | | | | | | |
|---|---|---|---|---|---|---|---|---|---|---|
| ATOM | 1796 | O | LYS A | 621 | 31.105 | 10.428 | 64.598 | 1.00 | 41.65 | O |

| | | | | | | | | | | | |
|---|---|---|---|---|---|---|---|---|---|---|---|
| ANISOU | 1796 | O | LYS A | 621 | 5441 | 4896 | 5488 | 39 | 90 | −189 | O |

| | | | | | | | | | | |
|---|---|---|---|---|---|---|---|---|---|---|
| ATOM | 1797 | CB | LYS A | 621 | 32.540 | 10.823 | 66.748 | 1.00 | 39.07 | C |

| | | | | | | | | | | | |
|---|---|---|---|---|---|---|---|---|---|---|---|
| ANISOU | 1797 | CB | LYS A | 621 | 5043 | 4693 | 5111 | 159 | 37 | −98 | C |

| | | | | | | | | | | |
|---|---|---|---|---|---|---|---|---|---|---|
| ATOM | 1798 | CG | LYS A | 621 | 31.583 | 10.196 | 67.684 | 1.00 | 42.00 | C |

| | | | | | | | | | | | |
|---|---|---|---|---|---|---|---|---|---|---|---|
| ANISOU | 1798 | CG | LYS A | 621 | 5286 | 5345 | 5328 | 30 | 128 | −25 | C |

| | | | | | | | | | | |
|---|---|---|---|---|---|---|---|---|---|---|
| ATOM | 1799 | CD | LYS A | 621 | 31.797 | 8.768 | 68.147 | 1.00 | 44.44 | C |

| | | | | | | | | | | | |
|---|---|---|---|---|---|---|---|---|---|---|---|
| ANISOU | 1799 | CD | LYS A | 621 | 5734 | 5463 | 5688 | 60 | 47 | 36 | C |

| | | | | | | | | | | |
|---|---|---|---|---|---|---|---|---|---|---|
| ATOM | 1800 | CE | LYS A | 621 | 30.905 | 7.777 | 67.425 | 1.00 | 45.57 | C |

| | | | | | | | | | | | |
|---|---|---|---|---|---|---|---|---|---|---|---|
| ANISOU | 1800 | CE | LYS A | 621 | 5760 | 5741 | 5813 | −48 | 20 | 10 | C |

| | | | | | | | | | | |
|---|---|---|---|---|---|---|---|---|---|---|
| ATOM | 1801 | NZ | LYS A | 621 | 31.375 | 6.374 | 67.661 | 1.00 | 46.21 | N |

| | | | | | | | | | | | |
|---|---|---|---|---|---|---|---|---|---|---|---|
| ANISOU | 1801 | NZ | LYS A | 621 | 5891 | 5768 | 5900 | −2 | −48 | −29 | N |
| TER | 1802 | | LYS A | 621 | | | | | | | |

| | | | | | | | | | | |
|---|---|---|---|---|---|---|---|---|---|---|
| ATOM | 1803 | N | PRO B | 20 | 5.110 | 13.180 | 40.537 | 1.00 | 61.48 | N |

| | | | | | | | | | | | |
|---|---|---|---|---|---|---|---|---|---|---|---|
| ANISOU | 1803 | N | PRO B | 20 | 7765 | 7820 | 7774 | −4 | 16 | −39 | N |

| | | | | | | | | | | |
|---|---|---|---|---|---|---|---|---|---|---|
| ATOM | 1804 | CA | PRO B | 20 | 5.653 | 13.683 | 41.845 | 1.00 | 61.11 | C |

| | | | | | | | | | | | |
|---|---|---|---|---|---|---|---|---|---|---|---|
| ANISOU | 1804 | CA | PRO B | 20 | 7685 | 7788 | 7747 | 43 | 9 | −2 | C |

| | | | | | | | | | | |
|---|---|---|---|---|---|---|---|---|---|---|
| ATOM | 1805 | C | PRO B | 20 | 6.859 | 12.823 | 42.221 | 1.00 | 60.40 | C |

| | | | | | | | | | | | |
|---|---|---|---|---|---|---|---|---|---|---|---|
| ANISOU | 1805 | C | PRO B | 20 | 7627 | 7719 | 7601 | 5 | 48 | −25 | C |

| | | | | | | | | | | |
|---|---|---|---|---|---|---|---|---|---|---|
| ATOM | 1806 | O | PRO B | 20 | 7.088 | 12.459 | 43.370 | 1.00 | 60.60 | O |

| | | | | | | | | | | | |
|---|---|---|---|---|---|---|---|---|---|---|---|
| ANISOU | 1806 | O | PRO B | 20 | 7696 | 7700 | 7627 | −3 | 21 | 21 | O |

| | | | | | | | | | | |
|---|---|---|---|---|---|---|---|---|---|---|
| ATOM | 1807 | N | GLU B | 21 | 7.642 | 12.514 | 41.182 | 1.00 | 59.31 | N |

| | | | | | | | | | | | |
|---|---|---|---|---|---|---|---|---|---|---|---|
| ANISOU | 1807 | N | GLU B | 21 | 7454 | 7536 | 7546 | 63 | −23 | 2 | N |

| | | | | | | | | | | |
|---|---|---|---|---|---|---|---|---|---|---|
| ATOM | 1808 | CA | GLU B | 21 | 8.823 | 11.690 | 41.325 | 1.00 | 56.74 | C |

| | | | | | | | | | | | |
|---|---|---|---|---|---|---|---|---|---|---|---|
| ANISOU | 1808 | CA | GLU B | 21 | 7183 | 7225 | 7151 | −114 | 12 | −50 | C |

| | | | | | | | | | | |
|---|---|---|---|---|---|---|---|---|---|---|
| ATOM | 1809 | C | GLU B | 21 | 10.081 | 12.447 | 41.693 | 1.00 | 54.30 | C |

| | | | | | | | | | | | |
|---|---|---|---|---|---|---|---|---|---|---|---|
| ANISOU | 1809 | C | GLU B | 21 | 6933 | 6896 | 6803 | 36 | 102 | 28 | C |

| | | | | | | | | | | |
|---|---|---|---|---|---|---|---|---|---|---|
| ATOM | 1810 | O | GLU B | 21 | 10.419 | 12.521 | 42.878 | 1.00 | 55.02 | O |

| | | | | | | | | | | | |
|---|---|---|---|---|---|---|---|---|---|---|---|
| ANISOU | 1810 | O | GLU B | 21 | 6999 | 7017 | 6888 | 13 | 14 | −39 | O |

| | | | | | | | | | | |
|---|---|---|---|---|---|---|---|---|---|---|
| ATOM | 1811 | N | THR B | 22 | 10.789 | 13.010 | 40.719 | 1.00 | 51.54 | N |

| | | | | | | | | | | | |
|---|---|---|---|---|---|---|---|---|---|---|---|
| ANISOU | 1811 | N | THR B | 22 | 6494 | 6494 | 6594 | −2 | −83 | −100 | N |

| | | | | | | | | | | |
|---|---|---|---|---|---|---|---|---|---|---|
| ATOM | 1812 | CA | THR B | 22 | 12.066 | 13.658 | 40.960 | 1.00 | 48.75 | C |

| | | | | | | | | | | | |
|---|---|---|---|---|---|---|---|---|---|---|---|
| ANISOU | 1812 | CA | THR B | 22 | 6360 | 6038 | 6125 | 63 | 2 | −91 | C |

| | | | | | | | | | | |
|---|---|---|---|---|---|---|---|---|---|---|
| ATOM | 1813 | C | THR B | 22 | 11.996 | 15.039 | 41.602 | 1.00 | 45.57 | C |

| | | | | | | | | | | | |
|---|---|---|---|---|---|---|---|---|---|---|---|
| ANISOU | 1813 | C | THR B | 22 | 5738 | 5853 | 5725 | 79 | 31 | 51 | C |

| | | | | | | | | | | |
|---|---|---|---|---|---|---|---|---|---|---|
| ATOM | 1814 | O | THR B | 22 | 13.004 | 15.512 | 42.136 | 1.00 | 43.92 | O |

| | | | | | | | | | | | |
|---|---|---|---|---|---|---|---|---|---|---|---|
| ANISOU | 1814 | O | THR B | 22 | 5775 | 5404 | 5507 | 115 | 143 | −37 | O |

| | | | | | | | | | | |
|---|---|---|---|---|---|---|---|---|---|---|
| ATOM | 1815 | CB | THR B | 22 | 12.928 | 13.823 | 39.683 | 1.00 | 49.62 | C |

| | | | | | | | | | | | |
|---|---|---|---|---|---|---|---|---|---|---|---|
| ANISOU | 1815 | CB | THR B | 22 | 6344 | 6270 | 6242 | 10 | 12 | 0 | C |

| | | | | | | | | | | |
|---|---|---|---|---|---|---|---|---|---|---|
| ATOM | 1816 | OG1 | THR B | 22 | 12.271 | 14.675 | 38.736 | 1.00 | 49.60 | O |

| | | | | | | | | | | | |
|---|---|---|---|---|---|---|---|---|---|---|---|
| ANISOU | 1816 | OG1 | THR B | 22 | 6338 | 6290 | 6217 | −68 | −66 | 16 | O |

| | | | | | | | | | | |
|---|---|---|---|---|---|---|---|---|---|---|
| ATOM | 1817 | CG2 | THR B | 22 | 13.218 | 12.478 | 39.042 | 1.00 | 50.11 | C |

| | | | | | | | | | | | |
|---|---|---|---|---|---|---|---|---|---|---|---|
| ANISOU | 1817 | CG2 | THR B | 22 | 6447 | 6295 | 6298 | −2 | 3 | −50 | C |

| | | | | | | | | | | |
|---|---|---|---|---|---|---|---|---|---|---|
| ATOM | 1818 | N | HIS B | 23 | 10.849 | 15.690 | 41.493 | 1.00 | 43.04 | N |

| | | | | | | | | | | | |
|---|---|---|---|---|---|---|---|---|---|---|---|
| ANISOU | 1818 | N | HIS B | 23 | 5682 | 5429 | 5241 | −4 | 35 | −57 | N |

| | | | | | | | | | | |
|---|---|---|---|---|---|---|---|---|---|---|
| ATOM | 1819 | CA | HIS B | 23 | 10.705 | 17.037 | 42.018 | 1.00 | 41.20 | C |

| | | | | | | | | | | | |
|---|---|---|---|---|---|---|---|---|---|---|---|
| ANISOU | 1819 | CA | HIS B | 23 | 5248 | 5383 | 5024 | 14 | −135 | 13 | C |

| | | | | | | | | | | |
|---|---|---|---|---|---|---|---|---|---|---|
| ATOM | 1820 | C | HIS B | 23 | 9.853 | 17.065 | 43.283 | 1.00 | 38.16 | C |

| | | | | | | | | | | | |
|---|---|---|---|---|---|---|---|---|---|---|---|
| ANISOU | 1820 | C | HIS B | 23 | 4955 | 4794 | 4752 | 12 | −43 | −40 | C |

| | | | | | | | | | | |
|---|---|---|---|---|---|---|---|---|---|---|
| ATOM | 1821 | O | HIS B | 23 | 9.121 | 16.125 | 43.560 | 1.00 | 36.19 | O |

| | | | | | | | | | | | |
|---|---|---|---|---|---|---|---|---|---|---|---|
| ANISOU | 1821 | O | HIS B | 23 | 4696 | 4817 | 4237 | 58 | 125 | −171 | O |

| | | | | | | | | | | |
|---|---|---|---|---|---|---|---|---|---|---|
| ATOM | 1822 | CB | HIS B | 23 | 10.117 | 17.932 | 40.916 | 1.00 | 43.73 | C |

| | | | | | | | | | | | |
|---|---|---|---|---|---|---|---|---|---|---|---|
| ANISOU | 1822 | CB | HIS B | 23 | 5636 | 5572 | 5405 | 81 | −69 | 83 | C |

| | | | | | | | | | | |
|---|---|---|---|---|---|---|---|---|---|---|
| ATOM | 1823 | CG | HIS B | 23 | 11.122 | 18.006 | 39.795 | 1.00 | 45.32 | C |

| | | | | | | | | | | | |
|---|---|---|---|---|---|---|---|---|---|---|---|
| ANISOU | 1823 | CG | HIS B | 23 | 5754 | 5826 | 5639 | 44 | 32 | 60 | C |

| | | | | | | | | | | |
|---|---|---|---|---|---|---|---|---|---|---|
| ATOM | 1824 | ND1 | HIS B | 23 | 10.988 | 17.306 | 38.613 | 1.00 | 45.91 | N |

| | | | | | | | | | | | |
|---|---|---|---|---|---|---|---|---|---|---|---|
| ANISOU | 1824 | ND1 | HIS B | 23 | 5844 | 5796 | 5803 | 33 | −27 | −28 | N |

| | | | | | | | | | | |
|---|---|---|---|---|---|---|---|---|---|---|
| ATOM | 1825 | CD2 | HIS B | 23 | 12.289 | 18.675 | 39.727 | 1.00 | 45.45 | C |

| | | | | | | | | | | | |
|---|---|---|---|---|---|---|---|---|---|---|---|
| ANISOU | 1825 | CD2 | HIS B | 23 | 5678 | 5868 | 5723 | 47 | −88 | 35 | C |

| | | | | | | | | | | |
|---|---|---|---|---|---|---|---|---|---|---|
| ATOM | 1826 | CE1 | HIS B | 23 | 12.038 | 17.579 | 37.860 | 1.00 | 46.16 | C |

| | | | | | | | | | | | |
|---|---|---|---|---|---|---|---|---|---|---|---|
| ANISOU | 1826 | CE1 | HIS B | 23 | 5833 | 5845 | 5861 | 27 | −14 | 18 | C |

| | | | | | | | | | | |
|---|---|---|---|---|---|---|---|---|---|---|
| ATOM | 1827 | NE2 | HIS B | 23 | 12.853 | 18.398 | 38.510 | 1.00 | 45.79 | N |

| | | | | | | | | | | | |
|---|---|---|---|---|---|---|---|---|---|---|---|
| ANISOU | 1827 | NE2 | HIS B | 23 | 5779 | 5879 | 5742 | 73 | −56 | 12 | N |

| | | | | | | | | | | |
|---|---|---|---|---|---|---|---|---|---|---|
| ATOM | 1828 | N | ILE B | 24 | 10.037 | 18.130 | 44.055 | 1.00 | 35.73 | N |

| | | | | | | | | | | | |
|---|---|---|---|---|---|---|---|---|---|---|---|
| ANISOU | 1828 | N | ILE B | 24 | 4644 | 4712 | 4221 | 46 | 65 | 96 | N |

| | | | | | | | | | | |
|---|---|---|---|---|---|---|---|---|---|---|
| ATOM | 1829 | CA | ILE B | 24 | 9.266 | 18.228 | 45.309 | 1.00 | 34.64 | C |

| | | | | | | | | | | | |
|---|---|---|---|---|---|---|---|---|---|---|---|
| ANISOU | 1829 | CA | ILE B | 24 | 4411 | 4542 | 4207 | 59 | 2 | −16 | C |

| | | | | | | | | | | |
|---|---|---|---|---|---|---|---|---|---|---|
| ATOM | 1830 | C | ILE B | 24 | 8.755 | 19.636 | 45.485 | 1.00 | 33.36 | C |

| | | | | | | | | | | | |
|---|---|---|---|---|---|---|---|---|---|---|---|
| ANISOU | 1830 | C | ILE B | 24 | 4205 | 4508 | 3964 | −13 | 65 | −13 | C |

| | | | | | | | | | | |
|---|---|---|---|---|---|---|---|---|---|---|
| ATOM | 1831 | O | ILE B | 24 | 9.443 | 20.604 | 45.140 | 1.00 | 33.90 | O |

| | | | | | | | | | | | |
|---|---|---|---|---|---|---|---|---|---|---|---|
| ANISOU | 1831 | O | ILE B | 24 | 4417 | 4464 | 4000 | 86 | 238 | −41 | O |

| | | | | | | | | | | |
|---|---|---|---|---|---|---|---|---|---|---|
| ATOM | 1832 | CB | ILE B | 24 | 10.176 | 17.769 | 46.473 | 1.00 | 34.49 | C |

| | | | | | | | | | | | |
|---|---|---|---|---|---|---|---|---|---|---|---|
| ANISOU | 1832 | CB | ILE B | 24 | 4433 | 4445 | 4229 | 47 | −7 | 34 | C |

| | | | | | | | | | | |
|---|---|---|---|---|---|---|---|---|---|---|
| ATOM | 1833 | CG1 | ILE B | 24 | 9.432 | 17.740 | 47.820 | 1.00 | 34.59 | C |

| | | | | | | | | | | | |
|---|---|---|---|---|---|---|---|---|---|---|---|
| ANISOU | 1833 | CG1 | ILE B | 24 | 4397 | 4470 | 4275 | 31 | 11 | −4 | C |

| | | | | | | | | | | |
|---|---|---|---|---|---|---|---|---|---|---|
| ATOM | 1834 | CG2 | ILE B | 24 | 11.431 | 18.624 | 46.600 | 1.00 | 34.31 | C |

| | | | | | | | | | | | |
|---|---|---|---|---|---|---|---|---|---|---|---|
| ANISOU | 1834 | CG2 | ILE B | 24 | 4362 | 4496 | 4177 | 83 | 48 | 35 | C |

| | | | | | | | | | | |
|---|---|---|---|---|---|---|---|---|---|---|
| ATOM | 1835 | CD1 | ILE B | 24 | 10.315 | 17.225 | 48.953 | 1.00 | 34.94 | C |

| | | | | | | | | | | | |
|---|---|---|---|---|---|---|---|---|---|---|---|
| ANISOU | 1835 | CD1 | ILE B | 24 | 4470 | 4561 | 4243 | −54 | −32 | 4 | C |

| | | | | | | | | | | |
|---|---|---|---|---|---|---|---|---|---|---|
| ATOM | 1836 | N | ASN B | 25 | 7.552 | 19.772 | 46.042 | 1.00 | 31.98 | N |

| | | | | | | | | | | | |
|---|---|---|---|---|---|---|---|---|---|---|---|
| ANISOU | 1836 | N | ASN B | 25 | 4085 | 4308 | 3759 | 72 | −28 | 5 | N |

| | | | | | | | | | | |
|---|---|---|---|---|---|---|---|---|---|---|
| ATOM | 1837 | CA | ASN B | 25 | 7.014 | 21.082 | 46.374 | 1.00 | 31.71 | C |

| | | | | | | | | | | | |
|---|---|---|---|---|---|---|---|---|---|---|---|
| ANISOU | 1837 | CA | ASN B | 25 | 4157 | 4165 | 3725 | −46 | −15 | 73 | C |

| | | | | | | | | | | |
|---|---|---|---|---|---|---|---|---|---|---|
| ATOM | 1838 | C | ASN B | 25 | 7.301 | 21.371 | 47.863 | 1.00 | 31.16 | C |

| | | | | | | | | | | | |
|---|---|---|---|---|---|---|---|---|---|---|---|
| ANISOU | 1838 | C | ASN B | 25 | 4094 | 4041 | 3705 | 4 | 67 | 41 | C |

| | | | | | | | | | | |
|---|---|---|---|---|---|---|---|---|---|---|
| ATOM | 1839 | O | ASN B | 25 | 7.057 | 20.526 | 48.734 | 1.00 | 30.08 | O |

| | | | | | | | | | | | |
|---|---|---|---|---|---|---|---|---|---|---|---|
| ANISOU | 1839 | O | ASN B | 25 | 4073 | 4004 | 3352 | 145 | −24 | −10 | O |

| | | | | | | | | | | |
|---|---|---|---|---|---|---|---|---|---|---|
| ATOM | 1840 | CB | ASN B | 25 | 5.512 | 21.160 | 46.129 | 1.00 | 33.14 | C |

| | | | | | | | | | | | |
|---|---|---|---|---|---|---|---|---|---|---|---|
| ANISOU | 1840 | CB | ASN B | 25 | 4208 | 4419 | 3965 | 33 | −43 | 23 | C |

| | | | | | | | | | | |
|---|---|---|---|---|---|---|---|---|---|---|
| ATOM | 1841 | CG | ASN B | 25 | 5.198 | 20.951 | 44.637 | 1.00 | 33.80 | C |

| | | | | | | | | | | | |
|---|---|---|---|---|---|---|---|---|---|---|---|
| ANISOU | 1841 | CG | ASN B | 25 | 4382 | 4484 | 3976 | 27 | −34 | 58 | C |

| | | | | | | | | | | |
|---|---|---|---|---|---|---|---|---|---|---|
| ATOM | 1842 | OD1 | ASN B | 25 | 5.366 | 21.915 | 43.902 | 1.00 | 34.49 | O |

| | | | | | | | | | | | |
|---|---|---|---|---|---|---|---|---|---|---|---|
| ANISOU | 1842 | OD1 | ASN B | 25 | 4398 | 4797 | 3911 | 21 | −100 | 212 | O |

| | | | | | | | | | | |
|---|---|---|---|---|---|---|---|---|---|---|
| ATOM | 1843 | ND2 | ASN B | 25 | 4.800 | 19.774 | 44.226 | 1.00 | 33.52 | N |

| | | | | | | | | | | | |
|---|---|---|---|---|---|---|---|---|---|---|---|
| ANISOU | 1843 | ND2 | ASN B | 25 | 4360 | 4642 | 3736 | −70 | −90 | 18 | N |

| | | | | | | | | | | |
|---|---|---|---|---|---|---|---|---|---|---|
| ATOM | 1844 | N | LEU B | 26 | 7.859 | 22.537 | 48.128 | 1.00 | 29.72 | N |

| | | | | | | | | | | | |
|---|---|---|---|---|---|---|---|---|---|---|---|
| ANISOU | 1844 | N | LEU B | 26 | 3737 | 4092 | 3461 | 10 | 49 | 18 | N |

| | | | | | | | | | | |
|---|---|---|---|---|---|---|---|---|---|---|
| ATOM | 1845 | CA | LEU B | 26 | 8.206 | 22.949 | 49.479 | 1.00 | 29.18 | C |

| | | | | | | | | | | | |
|---|---|---|---|---|---|---|---|---|---|---|---|
| ANISOU | 1845 | CA | LEU B | 26 | 3705 | 3863 | 3519 | −15 | 26 | −39 | C |

| | | | | | | | | | | |
|---|---|---|---|---|---|---|---|---|---|---|
| ATOM | 1846 | C | LEU B | 26 | 7.736 | 24.371 | 49.741 | 1.00 | 30.72 | C |

| | | | | | | | | | | | |
|---|---|---|---|---|---|---|---|---|---|---|---|
| ANISOU | 1846 | C | LEU B | 26 | 4018 | 3969 | 3687 | 89 | 27 | −27 | C |

| | | | | | | | | | | |
|---|---|---|---|---|---|---|---|---|---|---|
| ATOM | 1847 | O | LEU B | 26 | 7.687 | 25.217 | 48.846 | 1.00 | 31.50 | O |

| | | | | | | | | | | | |
|---|---|---|---|---|---|---|---|---|---|---|---|
| ANISOU | 1847 | O | LEU B | 26 | 4314 | 4057 | 3598 | 177 | 171 | −39 | O |

| | | | | | | | | | | |
|---|---|---|---|---|---|---|---|---|---|---|
| ATOM | 1848 | CB | LEU B | 26 | 9.731 | 22.863 | 49.685 | 1.00 | 28.96 | C |

| | | | | | | | | | | | |
|---|---|---|---|---|---|---|---|---|---|---|---|
| ANISOU | 1848 | CB | LEU B | 26 | 3717 | 3871 | 3416 | 18 | 0 | 33 | C |

| | | | | | | | | | | |
|---|---|---|---|---|---|---|---|---|---|---|
| ATOM | 1849 | CG | LEU B | 26 | 10.331 | 21.453 | 49.656 | 1.00 | 28.32 | C |

| | | | | | | | | | | | |
|---|---|---|---|---|---|---|---|---|---|---|---|
| ANISOU | 1849 | CG | LEU B | 26 | 3564 | 3813 | 3382 | −41 | −20 | −55 | C |

| | | | | | | | | | | |
|---|---|---|---|---|---|---|---|---|---|---|
| ATOM | 1850 | CD1 | LEU B | 26 | 11.866 | 21.481 | 49.715 | 1.00 | 29.50 | C |

| | | | | | | | | | | | |
|---|---|---|---|---|---|---|---|---|---|---|---|
| ANISOU | 1850 | CD1 | LEU B | 26 | 3586 | 3992 | 3629 | −51 | −21 | −27 | C |

| | | | | | | | | | | |
|---|---|---|---|---|---|---|---|---|---|---|
| ATOM | 1851 | CD2 | LEU B | 26 | 9.767 | 20.542 | 50.729 | 1.00 | 29.13 | C |

| | | | | | | | | | | | |
|---|---|---|---|---|---|---|---|---|---|---|---|
| ANISOU | 1851 | CD2 | LEU B | 26 | 3629 | 3955 | 3484 | −52 | −64 | 15 | C |

| | | | | | | | | | | |
|---|---|---|---|---|---|---|---|---|---|---|
| ATOM | 1852 | N | LYS B | 27 | 7.401 | 24.629 | 51.014 | 1.00 | 29.78 | N |

| | | | | | | | | | | | |
|---|---|---|---|---|---|---|---|---|---|---|---|
| ANISOU | 1852 | N | LYS B | 27 | 3771 | 3974 | 3572 | −27 | −52 | −9 | N |

| | | | | | | | | | | |
|---|---|---|---|---|---|---|---|---|---|---|
| ATOM | 1853 | CA | LYS B | 27 | 7.012 | 25.998 | 51.363 | 1.00 | 30.79 | C |

| | | | | | | | | | | | |
|---|---|---|---|---|---|---|---|---|---|---|---|
| ANISOU | 1853 | CA | LYS B | 27 | 3892 | 4060 | 3746 | −9 | −95 | −64 | C |

| | | | | | | | | | | |
|---|---|---|---|---|---|---|---|---|---|---|
| ATOM | 1854 | C | LYS B | 27 | 8.038 | 26.495 | 52.384 | 1.00 | 29.56 | C |

| | | | | | | | | | | | |
|---|---|---|---|---|---|---|---|---|---|---|---|
| ANISOU | 1854 | C | LYS B | 27 | 3726 | 3835 | 3668 | 24 | 6 | −100 | C |

| | | | | | | | | | | |
|---|---|---|---|---|---|---|---|---|---|---|
| ATOM | 1855 | O | LYS B | 27 | 8.414 | 25.686 | 53.235 | 1.00 | 29.68 | O |

| | | | | | | | | | | | |
|---|---|---|---|---|---|---|---|---|---|---|---|
| ANISOU | 1855 | O | LYS B | 27 | 3728 | 3844 | 3704 | −22 | −156 | −112 | O |

| | | | | | | | | | | |
|---|---|---|---|---|---|---|---|---|---|---|
| ATOM | 1856 | CB | LYS B | 27 | 5.631 | 26.014 | 51.972 | 1.00 | 32.36 | C |

| | | | | | | | | | | | |
|---|---|---|---|---|---|---|---|---|---|---|---|
| ANISOU | 1856 | CB | LYS B | 27 | 3900 | 4374 | 4022 | −72 | −108 | 22 | C |

| | | | | | | | | | | |
|---|---|---|---|---|---|---|---|---|---|---|
| ATOM | 1857 | CG | LYS B | 27 | 5.156 | 27.280 | 52.653 | 1.00 | 35.05 | C |

| | | | | | | | | | | | |
|---|---|---|---|---|---|---|---|---|---|---|---|
| ANISOU | 1857 | CG | LYS B | 27 | 4381 | 4510 | 4427 | 67 | −44 | −26 | C |

| | | | | | | | | | | |
|---|---|---|---|---|---|---|---|---|---|---|
| ATOM | 1858 | CD | LYS B | 27 | 3.673 | 27.065 | 52.992 | 1.00 | 36.85 | C |

| | | | | | | | | | | | |
|---|---|---|---|---|---|---|---|---|---|---|---|
| ANISOU | 1858 | CD | LYS B | 27 | 4419 | 4881 | 4702 | 53 | −45 | 13 | C |

| | | | | | | | | | | |
|---|---|---|---|---|---|---|---|---|---|---|
| ATOM | 1859 | CB | LYS B | 27 | 3.416 | 27.273 | 54.463 | 1.00 | 38.44 | C |

| | | | | | | | | | | | |
|---|---|---|---|---|---|---|---|---|---|---|---|
| ANISOU | 1859 | CB | LYS B | 27 | 4810 | 5039 | 4757 | 68 | −37 | 18 | C |

| | | | | | | | | | | |
|---|---|---|---|---|---|---|---|---|---|---|
| ATOM | 1860 | NZ | LYS B | 27 | 1.957 | 27.453 | 54.704 | 1.00 | 38.50 | N |

| | | | | | | | | | | | |
|---|---|---|---|---|---|---|---|---|---|---|---|
| ANISOU | 1860 | NZ | LYS B | 27 | 4762 | 5081 | 4784 | −23 | 50 | −3 | N |

| | | | | | | | | | | |
|---|---|---|---|---|---|---|---|---|---|---|
| ATOM | 1861 | N | VAL B | 28 | 8.462 | 27.741 | 52.287 | 1.00 | 28.44 | N |

| | | | | | | | | | | | |
|---|---|---|---|---|---|---|---|---|---|---|---|
| ANISOU | 1861 | N | VAL B | 28 | 3494 | 3850 | 3460 | 101 | 98 | −40 | N |

| | | | | | | | | | | |
|---|---|---|---|---|---|---|---|---|---|---|
| ATOM | 1862 | CA | VAL B | 28 | 9.336 | 28.264 | 53.341 | 1.00 | 28.28 | C |

| | | | | | | | | | | | |
|---|---|---|---|---|---|---|---|---|---|---|---|
| ANISOU | 1862 | CA | VAL B | 28 | 3600 | 3584 | 3560 | 109 | 12 | −89 | C |

| | | | | | | | | | | |
|---|---|---|---|---|---|---|---|---|---|---|
| ATOM | 1863 | C | VAL B | 28 | 8.510 | 29.348 | 54.057 | 1.00 | 28.57 | C |

| | | | | | | | | | | | |
|---|---|---|---|---|---|---|---|---|---|---|---|
| ANISOU | 1863 | C | VAL B | 28 | 3640 | 3685 | 3532 | 117 | 141 | −49 | C |

| | | | | | | | | | | |
|---|---|---|---|---|---|---|---|---|---|---|
| ATOM | 1864 | O | VAL B | 28 | 7.940 | 30.162 | 53.332 | 1.00 | 29.58 | O |

| | | | | | | | | | | | |
|---|---|---|---|---|---|---|---|---|---|---|---|
| ANISOU | 1864 | O | VAL B | 28 | 3832 | 3778 | 3631 | 302 | 89 | −128 | O |

| | | | | | | | | | | |
|---|---|---|---|---|---|---|---|---|---|---|
| ATOM | 1865 | CB | VAL B | 28 | 10.636 | 28.862 | 52.835 | 1.00 | 28.62 | C |

| | | | | | | | | | | | |
|---|---|---|---|---|---|---|---|---|---|---|---|
| ANISOU | 1865 | CB | VAL B | 28 | 3723 | 3645 | 3508 | 26 | −11 | −37 | C |

| | | | | | | | | | | |
|---|---|---|---|---|---|---|---|---|---|---|
| ATOM | 1866 | CG1 | VAL B | 28 | 11.509 | 29.407 | 53.974 | 1.00 | 28.63 | C |

| | | | | | | | | | | | |
|---|---|---|---|---|---|---|---|---|---|---|---|
| ANISOU | 1866 | CG1 | VAL B | 28 | 3864 | 3620 | 3394 | 51 | −71 | 70 | C |

| | | | | | | | | | | |
|---|---|---|---|---|---|---|---|---|---|---|
| ATOM | 1867 | CG2 | VAL B | 28 | 11.430 | 27.758 | 52.137 | 1.00 | 28.83 | C |

| | | | | | | | | | | | |
|---|---|---|---|---|---|---|---|---|---|---|---|
| ANISOU | 1867 | CG2 | VAL B | 28 | 3817 | 3688 | 3451 | 131 | 38 | 46 | C |

| | | | | | | | | | | |
|---|---|---|---|---|---|---|---|---|---|---|
| ATOM | 1888 | N | SER B | 29 | 8.475 | 29.305 | 55.375 | 1.00 | 27.01 | N |

| | | | | | | | | | | | |
|---|---|---|---|---|---|---|---|---|---|---|---|
| ANISOU | 1888 | N | SER B | 29 | 3435 | 3337 | 3488 | 97 | 26 | −78 | N |

| | | | | | | | | | | |
|---|---|---|---|---|---|---|---|---|---|---|
| ATOM | 1869 | CA | SER B | 29 | 7.676 | 30.330 | 56.066 | 1.00 | 27.64 | C |

| | | | | | | | | | | | |
|---|---|---|---|---|---|---|---|---|---|---|---|
| ANISOU | 1869 | CA | SER B | 29 | 3494 | 3515 | 3494 | 39 | 146 | −118 | C |

| | | | | | | | | | | |
|---|---|---|---|---|---|---|---|---|---|---|
| ATOM | 1870 | C | SER B | 29 | 8.450 | 30.928 | 57.238 | 1.00 | 27.95 | C |

| | | | | | | | | | | | |
|---|---|---|---|---|---|---|---|---|---|---|---|
| ANISOU | 1870 | C | SER B | 29 | 3356 | 3719 | 3545 | 60 | 15 | −4 | C |

| | | | | | | | | | | |
|---|---|---|---|---|---|---|---|---|---|---|
| ATOM | 1871 | O | SER B | 29 | 9.036 | 30.132 | 57.935 | 1.00 | 27.98 | O |

| | | | | | | | | | | | |
|---|---|---|---|---|---|---|---|---|---|---|---|
| ANISOU | 1871 | O | SER B | 29 | 3400 | 3826 | 3404 | 5 | 20 | 82 | O |

| | | | | | | | | | | |
|---|---|---|---|---|---|---|---|---|---|---|
| ATOM | 1872 | CB | SER B | 29 | 6.434 | 29.666 | 56.688 | 1.00 | 27.35 | C |

| | | | | | | | | | | | |
|---|---|---|---|---|---|---|---|---|---|---|---|
| ANISOU | 1872 | CB | SER B | 29 | 3348 | 3631 | 3414 | −28 | 5 | −133 | C |

| | | | | | | | | | | |
|---|---|---|---|---|---|---|---|---|---|---|
| ATOM | 1873 | OG | SER B | 29 | 5.618 | 30.700 | 57.245 | 1.00 | 28.47 | O |

| | | | | | | | | | | | |
|---|---|---|---|---|---|---|---|---|---|---|---|
| ANISOU | 1873 | OG | SER B | 29 | 3408 | 3748 | 3660 | 108 | −222 | −241 | O |

| | | | | | | | | | | |
|---|---|---|---|---|---|---|---|---|---|---|
| ATOM | 1874 | N | ASP B | 30 | 8.296 | 32.232 | 57.468 | 1.00 | 29.97 | N |

| | | | | | | | | | | | |
|---|---|---|---|---|---|---|---|---|---|---|---|
| ANISOU | 1874 | N | ASP B | 30 | 3855 | 3745 | 3787 | −8 | 59 | −15 | N |

| | | | | | | | | | | |
|---|---|---|---|---|---|---|---|---|---|---|
| ATOM | 1875 | CA | ASP B | 30 | 8.865 | 32.804 | 58.687 | 1.00 | 31.94 | C |

| | | | | | | | | | | | |
|---|---|---|---|---|---|---|---|---|---|---|---|
| ANISOU | 1875 | CA | ASP B | 30 | 4057 | 4063 | 4016 | 44 | −190 | 1 | C |

| | | | | | | | | | | |
|---|---|---|---|---|---|---|---|---|---|---|
| ATOM | 1876 | C | ASP B | 30 | 7.739 | 33.119 | 59.670 | 1.00 | 32.68 | C |

| | | | | | | | | | | | |
|---|---|---|---|---|---|---|---|---|---|---|---|
| ANISOU | 1876 | C | ASP B | 30 | 4366 | 4094 | 3957 | −35 | −20 | 156 | C |

| | | | | | | | | | | |
|---|---|---|---|---|---|---|---|---|---|---|
| ATOM | 1877 | O | ASP B | 30 | 7.947 | 33.860 | 60.627 | 1.00 | 33.73 | O |

| | | | | | | | | | | | |
|---|---|---|---|---|---|---|---|---|---|---|---|
| ANISOU | 1877 | O | ASP B | 30 | 4556 | 4160 | 4100 | 36 | −107 | 80 | O |

| | | | | | | | | | | |
|---|---|---|---|---|---|---|---|---|---|---|
| ATOM | 1878 | CB | ASP B | 30 | 9.710 | 34.046 | 58.429 | 1.00 | 32.87 | C |

| | | | | | | | | | | | |
|---|---|---|---|---|---|---|---|---|---|---|---|
| ANISOU | 1878 | CB | ASP B | 30 | 4267 | 4038 | 4184 | 63 | −61 | 43 | C |

| | | | | | | | | | | |
|---|---|---|---|---|---|---|---|---|---|---|
| ATOM | 1879 | CG | ASP B | 30 | 8.915 | 35.173 | 57.776 | 1.00 | 33.72 | C |

| | | | | | | | | | | | |
|---|---|---|---|---|---|---|---|---|---|---|---|
| ANISOU | 1879 | CG | ASP B | 30 | 4407 | 4149 | 4259 | 55 | −90 | 120 | C |

| | | | | | | | | | | |
|---|---|---|---|---|---|---|---|---|---|---|
| ATOM | 1880 | OD1 | ASP B | 30 | 7.684 | 35.039 | 57.697 | 1.00 | 33.45 | O |

| | | | | | | | | | | | |
|---|---|---|---|---|---|---|---|---|---|---|---|
| ANISOU | 1880 | OD1 | ASP B | 30 | 4354 | 4133 | 4221 | 268 | −98 | 101 | O |

| | | | | | | | | | | |
|---|---|---|---|---|---|---|---|---|---|---|
| ATOM | 1881 | OD2 | ASP B | 30 | 9.609 | 36.136 | 57.394 | 1.00 | 34.14 | O |

| | | | | | | | | | | | |
|---|---|---|---|---|---|---|---|---|---|---|---|
| ANISOU | 1881 | OD2 | ASP B | 30 | 4575 | 4060 | 4337 | 21 | −154 | 133 | O |

| | | | | | | | | | | |
|---|---|---|---|---|---|---|---|---|---|---|
| ATOM | 1882 | N | GLY B | 31 | 6.523 | 32.650 | 59.366 | 1.00 | 33.99 | N |

| | | | | | | | | | | | |
|---|---|---|---|---|---|---|---|---|---|---|---|
| ANISOU | 1882 | N | GLY B | 31 | 4279 | 4372 | 4263 | 75 | −84 | 201 | N |

| | | | | | | | | | | |
|---|---|---|---|---|---|---|---|---|---|---|
| ATOM | 1883 | CA | GLY B | 31 | 5.377 | 32.943 | 60.232 | 1.00 | 35.82 | C |

| | | | | | | | | | | | |
|---|---|---|---|---|---|---|---|---|---|---|---|
| ANISOU | 1883 | CA | GLY B | 31 | 4501 | 4628 | 4483 | −18 | 78 | 107 | C |

| | | | | | | | | | | |
|---|---|---|---|---|---|---|---|---|---|---|
| ATOM | 1884 | C | GLY B | 31 | 4.545 | 34.087 | 59.654 | 1.00 | 37.63 | C |

| | | | | | | | | | | | |
|---|---|---|---|---|---|---|---|---|---|---|---|
| ANISOU | 1884 | C | GLY B | 31 | 4733 | 4710 | 4856 | 84 | −56 | 16 | C |

| | | | | | | | | | | |
|---|---|---|---|---|---|---|---|---|---|---|
| ATOM | 1885 | O | GLY B | 31 | 3.410 | 34.293 | 60.088 | 1.00 | 38.14 | O |

| | | | | | | | | | | | |
|---|---|---|---|---|---|---|---|---|---|---|---|
| ANISOU | 1885 | O | GLY B | 31 | 4836 | 4778 | 4875 | 178 | 65 | 73 | O |

| | | | | | | | | | | |
|---|---|---|---|---|---|---|---|---|---|---|
| ATOM | 1886 | N | SER B | 32 | 5.075 | 34.839 | 58.696 | 1.00 | 39.48 | N |

| | | | | | | | | | | | |
|---|---|---|---|---|---|---|---|---|---|---|---|
| ANISOU | 1886 | N | SER B | 32 | 5045 | 5039 | 4915 | 24 | 53 | 48 | N |

| | | | | | | | | | | |
|---|---|---|---|---|---|---|---|---|---|---|
| ATOM | 1887 | CA | SER B | 32 | 4.230 | 35.913 | 58.137 | 1.00 | 41.83 | C |

| | | | | | | | | | | | |
|---|---|---|---|---|---|---|---|---|---|---|---|
| ANISOU | 1887 | CA | SER B | 32 | 5291 | 5287 | 5315 | 138 | −36 | 101 | C |

| | | | | | | | | | | |
|---|---|---|---|---|---|---|---|---|---|---|
| ATOM | 1888 | C | SER B | 32 | 4.388 | 35.939 | 56.629 | 1.00 | 41.91 | C |

| | | | | | | | | | | | |
|---|---|---|---|---|---|---|---|---|---|---|---|
| ANISOU | 1888 | C | SER B | 32 | 5324 | 5281 | 5318 | 57 | −33 | 44 | C |

| | | | | | | | | | | |
|---|---|---|---|---|---|---|---|---|---|---|
| ATOM | 1889 | O | SER B | 32 | 3.430 | 36.142 | 55.889 | 1.00 | 42.31 | O |

| | | | | | | | | | | | |
|---|---|---|---|---|---|---|---|---|---|---|---|
| ANISOU | 1889 | O | SER B | 32 | 5320 | 5338 | 5418 | −4 | −88 | −18 | O |

| | | | | | | | | | | |
|---|---|---|---|---|---|---|---|---|---|---|
| ATOM | 1890 | CB | SER B | 32 | 4.483 | 37.238 | 58.820 | 1.00 | 43.48 | C |

| | | | | | | | | | | | |
|---|---|---|---|---|---|---|---|---|---|---|---|
| ANISOU | 1890 | CB | SER B | 32 | 5586 | 5412 | 5522 | 47 | −40 | 17 | C |

| | | | | | | | | | | |
|---|---|---|---|---|---|---|---|---|---|---|
| ATOM | 1891 | OG | SER B | 32 | 5.833 | 37.625 | 58.826 | 1.00 | 45.44 | O |

| | | | | | | | | | | | |
|---|---|---|---|---|---|---|---|---|---|---|---|
| ANISOU | 1891 | OG | SER B | 32 | 5627 | 5812 | 5826 | 43 | −37 | 128 | O |

| | | | | | | | | | | |
|---|---|---|---|---|---|---|---|---|---|---|
| ATOM | 1892 | N | SER B | 33 | 5.603 | 35.698 | 56.179 | 1.00 | 41.29 | N |

| | | | | | | | | | | | |
|---|---|---|---|---|---|---|---|---|---|---|---|
| ANISOU | 1892 | N | SER B | 33 | 5337 | 5177 | 5173 | 70 | −18 | 43 | N |

| | | | | | | | | | | |
|---|---|---|---|---|---|---|---|---|---|---|
| ATOM | 1893 | CA | SER B | 33 | 5.909 | 35.639 | 54.751 | 1.00 | 40.95 | C |

| | | | | | | | | | | | |
|---|---|---|---|---|---|---|---|---|---|---|---|
| ANISOU | 1893 | CA | SER B | 33 | 5275 | 5148 | 5135 | 58 | −77 | −16 | C |

| | | | | | | | | | | |
|---|---|---|---|---|---|---|---|---|---|---|
| ATOM | 1894 | C | SER B | 33 | 6.145 | 34.181 | 54.379 | 1.00 | 39.85 | C |

| | | | | | | | | | | | |
|---|---|---|---|---|---|---|---|---|---|---|---|
| ANISOU | 1894 | C | SER B | 33 | 5060 | 5125 | 4957 | 89 | −75 | −27 | C |

| | | | | | | | | | | |
|---|---|---|---|---|---|---|---|---|---|---|
| ATOM | 1895 | O | SER B | 33 | 6.910 | 33.481 | 55.051 | 1.00 | 38.57 | O |

| | | | | | | | | | | | |
|---|---|---|---|---|---|---|---|---|---|---|---|
| ANISOU | 1895 | O | SER B | 33 | 4927 | 5004 | 4725 | −19 | −47 | −26 | O |

| | | | | | | | | | | |
|---|---|---|---|---|---|---|---|---|---|---|
| ATOM | 1896 | CB | SER B | 33 | 7.114 | 36.520 | 54.479 | 1.00 | 41.78 | C |

| | | | | | | | | | | | |
|---|---|---|---|---|---|---|---|---|---|---|---|
| ANISOU | 1896 | CB | SER B | 33 | 5333 | 5254 | 5287 | 3 | −43 | 24 | C |

| | | | | | | | | | | |
|---|---|---|---|---|---|---|---|---|---|---|
| ATOM | 1897 | OG | SER B | 33 | 7.800 | 36.180 | 53.287 | 1.00 | 43.51 | O |

| | | | | | | | | | | | |
|---|---|---|---|---|---|---|---|---|---|---|---|
| ANISOU | 1897 | OG | SER B | 33 | 5620 | 5484 | 5429 | 64 | 70 | −39 | O |

| | | | | | | | | | | |
|---|---|---|---|---|---|---|---|---|---|---|
| ATOM | 1898 | N | GLU B | 34 | 5.421 | 33.685 | 53.375 | 1.00 | 39.09 | N |

| | | | | | | | | | | | |
|---|---|---|---|---|---|---|---|---|---|---|---|
| ANISOU | 1898 | N | GLU B | 34 | 4914 | 5033 | 4906 | 136 | −64 | 19 | N |

| | | | | | | | | | | |
|---|---|---|---|---|---|---|---|---|---|---|
| ATOM | 1899 | CA | GLU B | 34 | 5.625 | 32.300 | 52.943 | 1.00 | 39.40 | C |

| | | | | | | | | | | | |
|---|---|---|---|---|---|---|---|---|---|---|---|
| ANISOU | 1899 | CA | GLU B | 34 | 5037 | 5061 | 4872 | 143 | −93 | −7 | C |

| | | | | | | | | | | |
|---|---|---|---|---|---|---|---|---|---|---|
| ATOM | 1900 | C | GLU B | 34 | 5.855 | 32.255 | 51.441 | 1.00 | 38.17 | C |

| | | | | | | | | | | | |
|---|---|---|---|---|---|---|---|---|---|---|---|
| ANISOU | 1900 | C | GLU B | 34 | 4759 | 4880 | 4865 | 143 | −9 | 44 | C |

| | | | | | | | | | | |
|---|---|---|---|---|---|---|---|---|---|---|
| ATOM | 1901 | O | GLU B | 34 | 5.247 | 33.013 | 50.670 | 1.00 | 37.97 | O |

| | | | | | | | | | | | |
|---|---|---|---|---|---|---|---|---|---|---|---|
| ANISOU | 1901 | O | GLU B | 34 | 4720 | 4965 | 4740 | 183 | −64 | 30 | O |

| | | | | | | | | | | |
|---|---|---|---|---|---|---|---|---|---|---|
| ATOM | 1902 | CB | GLU B | 34 | 4.538 | 31.363 | 53.413 | 1.00 | 42.32 | C |

| | | | | | | | | | | | |
|---|---|---|---|---|---|---|---|---|---|---|---|
| ANISOU | 1902 | CB | GLU B | 34 | 5384 | 5292 | 5403 | −37 | 18 | 26 | C |

| | | | | | | | | | | |
|---|---|---|---|---|---|---|---|---|---|---|
| ATOM | 1903 | CG | GLU B | 34 | 3.092 | 31.728 | 53.153 | 1.00 | 45.21 | C |

| | | | | | | | | | | | |
|---|---|---|---|---|---|---|---|---|---|---|---|
| ANISOU | 1903 | CG | GLU B | 34 | 5558 | 5793 | 5826 | 55 | −86 | −39 | C |

| | | | | | | | | | | |
|---|---|---|---|---|---|---|---|---|---|---|
| ATOM | 1904 | CD | GLU B | 34 | 2.231 | 31.106 | 54.254 | 1.00 | 47.57 | C |

| | | | | | | | | | | | |
|---|---|---|---|---|---|---|---|---|---|---|---|
| ANISOU | 1904 | CD | GLU B | 34 | 5946 | 6095 | 6033 | −38 | 45 | 64 | C |

| | | | | | | | | | | |
|---|---|---|---|---|---|---|---|---|---|---|
| ATOM | 1905 | OE1 | GLU B | 34 | 1.935 | 31.835 | 55.239 | 1.00 | 49.22 | O |

| | | | | | | | | | | | |
|---|---|---|---|---|---|---|---|---|---|---|---|
| ANISOU | 1905 | OE1 | GLU B | 34 | 6208 | 6370 | 6124 | −7 | 64 | −12 | O |

| | | | | | | | | | | |
|---|---|---|---|---|---|---|---|---|---|---|
| ATOM | 1906 | OE2 | GLU B | 34 | 1.882 | 29.922 | 54.132 | 1.00 | 47.63 | O |

| | | | | | | | | | | | |
|---|---|---|---|---|---|---|---|---|---|---|---|
| ANISOU | 1906 | OE2 | GLU B | 34 | 5936 | 6094 | 6066 | 10 | 45 | −17 | O |

| | | | | | | | | | | |
|---|---|---|---|---|---|---|---|---|---|---|
| ATOM | 1907 | N | ILE B | 35 | 6.854 | 31.475 | 51.029 | 1.00 | 36.43 | N |

| | | | | | | | | | | | |
|---|---|---|---|---|---|---|---|---|---|---|---|
| ANISOU | 1907 | N | ILE B | 35 | 4694 | 4738 | 4411 | 129 | −75 | 44 | N |

| | | | | | | | | | | |
|---|---|---|---|---|---|---|---|---|---|---|
| ATOM | 1908 | CA | ILE B | 35 | 7.209 | 31.364 | 49.611 | 1.00 | 35.38 | C |

| | | | | | | | | | | | |
|---|---|---|---|---|---|---|---|---|---|---|---|
| ANISOU | 1908 | CA | ILE B | 35 | 4485 | 4541 | 4415 | 115 | −44 | 85 | C |

| | | | | | | | | | | |
|---|---|---|---|---|---|---|---|---|---|---|
| ATOM | 1909 | C | ILE B | 35 | 7.201 | 29.890 | 49.233 | 1.00 | 33.87 | C |

| | | | | | | | | | | | |
|---|---|---|---|---|---|---|---|---|---|---|---|
| ANISOU | 1909 | C | ILE B | 35 | 4233 | 4495 | 4139 | 141 | −1 | 101 | C |

| | | | | | | | | | | |
|---|---|---|---|---|---|---|---|---|---|---|
| ATOM | 1910 | O | ILE B | 35 | 7.742 | 29.056 | 49.957 | 1.00 | 32.10 | O |

| | | | | | | | | | | | |
|---|---|---|---|---|---|---|---|---|---|---|---|
| ANISOU | 1910 | O | ILE B | 35 | 4048 | 4560 | 3589 | 101 | 205 | 166 | O |

| | | | | | | | | | | |
|---|---|---|---|---|---|---|---|---|---|---|
| ATOM | 1911 | CB | ILE B | 35 | 8.583 | 31.989 | 49.305 | 1.00 | 35.74 | C |

| | | | | | | | | | | | |
|---|---|---|---|---|---|---|---|---|---|---|---|
| ANISOU | 1911 | CB | ILE B | 35 | 4594 | 4558 | 4428 | 71 | 9 | 70 | C |

| | | | | | | | | | | |
|---|---|---|---|---|---|---|---|---|---|---|
| ATOM | 1912 | CG1 | ILE B | 35 | 8.655 | 33.440 | 49.757 | 1.00 | 35.99 | C |

| | | | | | | | | | | | |
|---|---|---|---|---|---|---|---|---|---|---|---|
| ANISOU | 1912 | CG1 | ILE B | 35 | 4649 | 4557 | 4470 | 20 | −50 | 107 | C |

| | | | | | | | | | | |
|---|---|---|---|---|---|---|---|---|---|---|
| ATOM | 1913 | CG2 | ILE B | 35 | 8.849 | 31.836 | 47.813 | 1.00 | 35.28 | C |

| | | | | | | | | | | | |
|---|---|---|---|---|---|---|---|---|---|---|---|
| ANISOU | 1913 | CG2 | ILE B | 35 | 4494 | 4515 | 4395 | 24 | 16 | 108 | C |

| | | | | | | | | | | |
|---|---|---|---|---|---|---|---|---|---|---|
| ATOM | 1914 | CD1 | ILE B | 35 | 10.009 | 34.086 | 49.738 | 1.00 | 37.60 | C |

| | | | | | | | | | | | |
|---|---|---|---|---|---|---|---|---|---|---|---|
| ANISOU | 1914 | CD1 | ILE B | 35 | 4766 | 4690 | 4831 | −38 | −50 | 51 | C |

| | | | | | | | | | | |
|---|---|---|---|---|---|---|---|---|---|---|
| ATOM | 1915 | N | PHE B | 36 | 6.546 | 29.539 | 48.130 | 1.00 | 33.78 | N |

| | | | | | | | | | | | |
|---|---|---|---|---|---|---|---|---|---|---|---|
| ANISOU | 1915 | N | PHE B | 36 | 4335 | 4443 | 4059 | 122 | 34 | 124 | N |

| | | | | | | | | | | |
|---|---|---|---|---|---|---|---|---|---|---|
| ATOM | 1916 | CA | PHE B | 36 | 6.443 | 28.183 | 47.636 | 1.00 | 34.11 | C |

| | | | | | | | | | | | |
|---|---|---|---|---|---|---|---|---|---|---|---|
| ANISOU | 1916 | CA | PHE B | 36 | 4385 | 4468 | 4107 | 66 | 50 | 100 | C |

| | | | | | | | | | | |
|---|---|---|---|---|---|---|---|---|---|---|
| ATOM | 1917 | C | PHE B | 36 | 7.487 | 27.964 | 46.537 | 1.00 | 33.99 | C |

| | | | | | | | | | | | |
|---|---|---|---|---|---|---|---|---|---|---|---|
| ANISOU | 1917 | C | PHE B | 36 | 4333 | 4388 | 4194 | 96 | 10 | 60 | C |

| | | | | | | | | | | |
|---|---|---|---|---|---|---|---|---|---|---|
| ATOM | 1918 | O | PHE B | 36 | 7.753 | 28.877 | 45.747 | 1.00 | 32.71 | O |

| | | | | | | | | | | | |
|---|---|---|---|---|---|---|---|---|---|---|---|
| ANISOU | 1918 | O | PHE B | 36 | 4170 | 4417 | 3843 | 126 | 108 | −43 | O |

| | | | | | | | | | | |
|---|---|---|---|---|---|---|---|---|---|---|
| ATOM | 1919 | CB | PHE B | 36 | 5.076 | 27.884 | 47.022 | 1.00 | 36.02 | C |

| | | | | | | | | | | | |
|---|---|---|---|---|---|---|---|---|---|---|---|
| ANISOU | 1919 | CB | PHE B | 36 | 4508 | 4717 | 4459 | 27 | −32 | 23 | C |

| | | | | | | | | | | |
|---|---|---|---|---|---|---|---|---|---|---|
| ATOM | 1920 | CG | PHE B | 36 | 3.984 | 27.916 | 48.065 | 1.00 | 37.58 | C |

| | | | | | | | | | | | |
|---|---|---|---|---|---|---|---|---|---|---|---|
| ANISOU | 1920 | CG | PHE B | 36 | 4635 | 4993 | 4653 | 18 | 54 | 10 | C |

| | | | | | | | | | | |
|---|---|---|---|---|---|---|---|---|---|---|
| ATOM | 1921 | CD1 | PHE B | 36 | 3.421 | 29.125 | 48.440 | 1.00 | 38.57 | C |

| | | | | | | | | | | | |
|---|---|---|---|---|---|---|---|---|---|---|---|
| ANISOU | 1921 | CD1 | PHE B | 36 | 4841 | 5031 | 4783 | 50 | −2 | −21 | C |

| | | | | | | | | | | |
|---|---|---|---|---|---|---|---|---|---|---|
| ATOM | 1922 | CD2 | PHE B | 36 | 3.556 | 26.746 | 48.651 | 1.00 | 38.52 | C |

| | | | | | | | | | | | |
|---|---|---|---|---|---|---|---|---|---|---|---|
| ANISOU | 1922 | CD2 | PHE B | 36 | 4818 | 4985 | 4834 | 13 | −1 | 42 | C |

| | | | | | | | | | | |
|---|---|---|---|---|---|---|---|---|---|---|
| ATOM | 1923 | CE1 | PHE B | 36 | 2.404 | 29.155 | 49.389 | 1.00 | 38.81 | C |

| | | | | | | | | | | | |
|---|---|---|---|---|---|---|---|---|---|---|---|
| ANISOU | 1923 | CE1 | PHE B | 36 | 4791 | 5081 | 4875 | 15 | 16 | −17 | C |

| | | | | | | | | | | |
|---|---|---|---|---|---|---|---|---|---|---|
| ATOM | 1924 | CE2 | PHE B | 36 | 2.540 | 26.776 | 49.605 | 1.00 | 39.51 | C |

| | | | | | | | | | | | |
|---|---|---|---|---|---|---|---|---|---|---|---|
| ANISOU | 1924 | CE2 | PHE B | 36 | 4963 | 5111 | 4939 | 25 | 77 | −31 | C |

| | | | | | | | | | | |
|---|---|---|---|---|---|---|---|---|---|---|
| ATOM | 1925 | CZ | PHE B | 36 | 1.981 | 27.980 | 49.968 | 1.00 | 39.14 | C |

| | | | | | | | | | | | |
|---|---|---|---|---|---|---|---|---|---|---|---|
| ANISOU | 1925 | CZ | PHE B | 36 | 4847 | 5070 | 4955 | 36 | 18 | 9 | C |

| | | | | | | | | | | |
|---|---|---|---|---|---|---|---|---|---|---|
| ATOM | 1926 | N | PHE B | 37 | 8.125 | 26.806 | 46.615 | 1.00 | 33.20 | N |

| | | | | | | | | | | | |
|---|---|---|---|---|---|---|---|---|---|---|---|
| ANISOU | 1926 | N | PHE B | 37 | 4255 | 4317 | 4042 | 72 | 31 | 11 | N |

| | | | | | | | | | | |
|---|---|---|---|---|---|---|---|---|---|---|
| ATOM | 1927 | CA | PHE B | 37 | 9.179 | 26.480 | 45.670 | 1.00 | 33.43 | C |

| | | | | | | | | | | | |
|---|---|---|---|---|---|---|---|---|---|---|---|
| ANISOU | 1927 | CA | PHE B | 37 | 4142 | 4413 | 4149 | 38 | 12 | 38 | C |

| | | | | | | | | | | |
|---|---|---|---|---|---|---|---|---|---|---|
| ATOM | 1928 | C | PHE B | 37 | 8.946 | 25.074 | 45.152 | 1.00 | 34.30 | C |

| | | | | | | | | | | | |
|---|---|---|---|---|---|---|---|---|---|---|---|
| ANISOU | 1928 | C | PHE B | 37 | 4313 | 4445 | 4274 | 30 | 3 | 4 | C |

| | | | | | | | | | | |
|---|---|---|---|---|---|---|---|---|---|---|
| ATOM | 1929 | O | PHE B | 37 | 8.320 | 24.252 | 45.809 | 1.00 | 35.32 | O |

| | | | | | | | | | | | |
|---|---|---|---|---|---|---|---|---|---|---|---|
| ANISOU | 1929 | O | PHE B | 37 | 4473 | 4673 | 4273 | −10 | 46 | 105 | O |

| | | | | | | | | | | |
|---|---|---|---|---|---|---|---|---|---|---|
| ATOM | 1930 | CB | PHE B | 37 | 10.555 | 26.496 | 46.355 | 1.00 | 32.86 | C |

| | | | | | | | | | | | |
|---|---|---|---|---|---|---|---|---|---|---|---|
| ANISOU | 1930 | CB | PHE B | 37 | 4077 | 4273 | 4135 | −8 | 53 | 22 | C |

| | | | | | | | | | | |
|---|---|---|---|---|---|---|---|---|---|---|
| ATOM | 1931 | CG | PHE B | 37 | 11.005 | 27.793 | 46.939 | 1.00 | 32.59 | C |

| | | | | | | | | | | | |
|---|---|---|---|---|---|---|---|---|---|---|---|
| ANISOU | 1931 | CG | PHE B | 37 | 3979 | 4294 | 4111 | −2 | 25 | 14 | C |

| | | | | | | | | | | |
|---|---|---|---|---|---|---|---|---|---|---|
| ATOM | 1932 | CD1 | PHE B | 37 | 10.725 | 29.128 | 48.253 | 1.00 | 31.78 | C |

| | | | | | | | | | | | |
|---|---|---|---|---|---|---|---|---|---|---|---|
| ANISOU | 1932 | CD1 | PHE B | 37 | 3870 | 4151 | 4053 | 19 | 14 | 56 | C |

| | | | | | | | | | | |
|---|---|---|---|---|---|---|---|---|---|---|
| ATOM | 1933 | CD2 | PHE B | 37 | 11.752 | 28.680 | 46.171 | 1.00 | 32.67 | C |

| | | | | | | | | | | | |
|---|---|---|---|---|---|---|---|---|---|---|---|
| ANISOU | 1933 | CD2 | PHE B | 37 | 4108 | 4218 | 4086 | −24 | 24 | 65 | C |

| | | | | | | | | | | |
|---|---|---|---|---|---|---|---|---|---|---|
| ATOM | 1934 | CE1 | PHE B | 37 | 11.151 | 29.334 | 48.782 | 1.00 | 32.32 | C |

| | | | | | | | | | | | |
|---|---|---|---|---|---|---|---|---|---|---|---|
| ANISOU | 1934 | CE1 | PHE B | 37 | 3985 | 4134 | 4160 | 36 | −7 | 73 | C |

| | | | | | | | | | | |
|---|---|---|---|---|---|---|---|---|---|---|
| ATOM | 1935 | CE2 | PHE B | 37 | 12.187 | 29.874 | 46.702 | 1.00 | 32.84 | C |

| | | | | | | | | | | | |
|---|---|---|---|---|---|---|---|---|---|---|---|
| ANISOU | 1935 | CE2 | PHE B | 37 | 4095 | 4228 | 4154 | −11 | 45 | 46 | C |

| | | | | | | | | | | |
|---|---|---|---|---|---|---|---|---|---|---|
| ATOM | 1936 | CZ | PHE B | 37 | 11.898 | 30.207 | 48.008 | 1.00 | 32.48 | C |

| | | | | | | | | | | | |
|---|---|---|---|---|---|---|---|---|---|---|---|
| ANISOU | 1936 | CZ | PHE B | 37 | 3993 | 4183 | 4165 | 33 | 37 | 68 | C |

| | | | | | | | | | | |
|---|---|---|---|---|---|---|---|---|---|---|
| ATOM | 1937 | N | LYS B | 38 | 9.514 | 24.772 | 43.986 | 1.00 | 34.78 | N |

| | | | | | | | | | | | |
|---|---|---|---|---|---|---|---|---|---|---|---|
| ANISOU | 1937 | N | LYS B | 38 | 4435 | 4520 | 4258 | −4 | 43 | 75 | N |

| | | | | | | | | | | |
|---|---|---|---|---|---|---|---|---|---|---|
| ATOM | 1938 | CA | LYS B | 38 | 9.412 | 23.419 | 43.418 | 1.00 | 35.03 | C |

| | | | | | | | | | | | |
|---|---|---|---|---|---|---|---|---|---|---|---|
| ANISOU | 1938 | CA | LYS B | 38 | 4434 | 4480 | 4394 | 56 | 77 | 24 | C |

| | | | | | | | | | | |
|---|---|---|---|---|---|---|---|---|---|---|
| ATOM | 1939 | C | LYS B | 38 | 10.835 | 23.151 | 42.930 | 1.00 | 35.27 | C |

| | | | | | | | | | | | |
|---|---|---|---|---|---|---|---|---|---|---|---|
| ANISOU | 1939 | C | LYS B | 38 | 4471 | 4531 | 4398 | 44 | 91 | 27 | C |

| | | | | | | | | | | |
|---|---|---|---|---|---|---|---|---|---|---|
| ATOM | 1940 | O | LYS B | 38 | 11.370 | 23.978 | 42.186 | 1.00 | 35.51 | O |

| | | | | | | | | | | | |
|---|---|---|---|---|---|---|---|---|---|---|---|
| ANISOU | 1940 | O | LYS B | 38 | 4617 | 4626 | 4251 | 185 | 116 | 109 | O |

| | | | | | | | | | | |
|---|---|---|---|---|---|---|---|---|---|---|
| ATOM | 1941 | CB | LYS B | 38 | 8.416 | 23.338 | 42.285 | 1.00 | 35.88 | C |

| | | | | | | | | | | | |
|---|---|---|---|---|---|---|---|---|---|---|---|
| ANISOU | 1941 | CB | LYS B | 38 | 4550 | 4670 | 4412 | 17 | 35 | 33 | C |

| | | | | | | | | | | |
|---|---|---|---|---|---|---|---|---|---|---|
| ATOM | 1942 | CG | LYS B | 38 | 8.187 | 21.954 | 41.684 | 1.00 | 37.63 | C |

| | | | | | | | | | | | |
|---|---|---|---|---|---|---|---|---|---|---|---|
| ANISOU | 1942 | CG | LYS B | 38 | 4886 | 4732 | 4681 | −13 | 59 | −34 | C |

| | | | | | | | | | | |
|---|---|---|---|---|---|---|---|---|---|---|
| ATOM | 1943 | CD | LYS B | 38 | 6.987 | 22.028 | 40.721 | 1.00 | 39.86 | C |

| | | | | | | | | | | | |
|---|---|---|---|---|---|---|---|---|---|---|---|
| ANISOU | 1943 | CD | LYS B | 38 | 5055 | 5177 | 4915 | −37 | −49 | −21 | C |

| | | | | | | | | | | |
|---|---|---|---|---|---|---|---|---|---|---|
| ATOM | 1944 | CE | LYS B | 38 | 6.566 | 20.634 | 40.272 | 1.00 | 40.85 | C |

| | | | | | | | | | | | |
|---|---|---|---|---|---|---|---|---|---|---|---|
| ANISOU | 1944 | CE | LYS B | 38 | 5294 | 5124 | 5104 | −54 | −17 | 44 | C |

| | | | | | | | | | | |
|---|---|---|---|---|---|---|---|---|---|---|
| ATOM | 1945 | NZ | LYS B | 38 | 5.525 | 20.674 | 39.200 | 1.00 | 41.09 | N |

| | | | | | | | | | | | |
|---|---|---|---|---|---|---|---|---|---|---|---|
| ANISOU | 1945 | NZ | LYS B | 38 | 5071 | 5212 | 5330 | −149 | −8 | −29 | N |

| | | | | | | | | | | |
|---|---|---|---|---|---|---|---|---|---|---|
| ATOM | 1946 | N | ILE B | 39 | 11.508 | 22.203 | 43.548 | 1.00 | 35.56 | N |

| | | | | | | | | | | | |
|---|---|---|---|---|---|---|---|---|---|---|---|
| ANISOU | 1946 | N | ILE B | 39 | 4572 | 4566 | 4374 | 74 | 103 | 99 | N |

| | | | | | | | | | | |
|---|---|---|---|---|---|---|---|---|---|---|
| ATOM | 1947 | CA | ILE B | 39 | 12.888 | 21.931 | 43.200 | 1.00 | 36.28 | C |

| | | | | | | | | | | | |
|---|---|---|---|---|---|---|---|---|---|---|---|
| ANISOU | 1947 | CA | ILE B | 39 | 4600 | 4666 | 4519 | 37 | 101 | 72 | C |

| | | | | | | | | | | |
|---|---|---|---|---|---|---|---|---|---|---|
| ATOM | 1948 | C | ILE B | 39 | 13.052 | 20.418 | 43.021 | 1.00 | 37.20 | C |

| | | | | | | | | | | | |
|---|---|---|---|---|---|---|---|---|---|---|---|
| ANISOU | 1948 | C | ILE B | 39 | 4815 | 4709 | 4610 | 40 | 17 | 22 | C |

| | | | | | | | | | | |
|---|---|---|---|---|---|---|---|---|---|---|
| ATOM | 1949 | O | ILE B | 39 | 12.144 | 19.633 | 43.269 | 1.00 | 37.22 | O |

| | | | | | | | | | | | |
|---|---|---|---|---|---|---|---|---|---|---|---|
| ANISOU | 1949 | O | ILE B | 39 | 5005 | 4663 | 4473 | 54 | 143 | 93 | O |

| | | | | | | | | | | |
|---|---|---|---|---|---|---|---|---|---|---|
| ATOM | 1950 | CB | ILE B | 39 | 13.934 | 22.407 | 44.215 | 1.00 | 36.31 | C |

| | | | | | | | | | | | |
|---|---|---|---|---|---|---|---|---|---|---|---|
| ANISOU | 1950 | CB | ILE B | 39 | 4685 | 4600 | 4509 | 50 | 91 | 42 | C |

| | | | | | | | | | | |
|---|---|---|---|---|---|---|---|---|---|---|
| ATOM | 1951 | CG1 | ILE B | 39 | 13.690 | 21.786 | 45.602 | 1.00 | 35.97 | C |

| | | | | | | | | | | | |
|---|---|---|---|---|---|---|---|---|---|---|---|
| ANISOU | 1951 | CG1 | ILE B | 39 | 4604 | 4581 | 4480 | 66 | 85 | 20 | C |

| | | | | | | | | | | |
|---|---|---|---|---|---|---|---|---|---|---|
| ATOM | 1952 | CG2 | ILE B | 39 | 13.984 | 23.926 | 44.280 | 1.00 | 36.35 | C |

| | | | | | | | | | | | |
|---|---|---|---|---|---|---|---|---|---|---|---|
| ANISOU | 1952 | CG2 | ILE B | 39 | 4587 | 4609 | 4617 | 94 | 42 | −2 | C |

| | | | | | | | | | | |
|---|---|---|---|---|---|---|---|---|---|---|
| ATOM | 1953 | CD1 | ILE B | 39 | 14.899 | 21.909 | 46.507 | 1.00 | 35.73 | C |

| | | | | | | | | | | | |
|---|---|---|---|---|---|---|---|---|---|---|---|
| ANISOU | 1953 | CD1 | ILE B | 39 | 4679 | 4488 | 4410 | 78 | 103 | −22 | C |

| | | | | | | | | | | |
|---|---|---|---|---|---|---|---|---|---|---|
| ATOM | 1954 | N | LYS B | 40 | 14.224 | 20.092 | 42.497 | 1.00 | 38.39 | N |

| | | | | | | | | | | | |
|---|---|---|---|---|---|---|---|---|---|---|---|
| ANISOU | 1954 | N | LYS B | 40 | 4885 | 4980 | 4723 | 40 | 64 | 64 | N |

| | | | | | | | | | | |
|---|---|---|---|---|---|---|---|---|---|---|
| ATOM | 1955 | CA | LYS B | 40 | 14.540 | 18.670 | 42.308 | 1.00 | 40.36 | C |

| | | | | | | | | | | | |
|---|---|---|---|---|---|---|---|---|---|---|---|
| ANISOU | 1955 | CA | LYS B | 40 | 5159 | 5087 | 5089 | 88 | 2 | −12 | C |

| | | | | | | | | | | |
|---|---|---|---|---|---|---|---|---|---|---|
| ATOM | 1956 | C | LYS B | 40 | 14.870 | 18.131 | 43.687 | 1.00 | 39.69 | C |

| | | | | | | | | | | | |
|---|---|---|---|---|---|---|---|---|---|---|---|
| ANISOU | 1956 | C | LYS B | 40 | 4975 | 5114 | 4991 | 100 | 91 | −60 | C |

| | | | | | | | | | | |
|---|---|---|---|---|---|---|---|---|---|---|
| ATOM | 1957 | O | LYS B | 40 | 15.482 | 18.878 | 44.467 | 1.00 | 39.08 | O |

| | | | | | | | | | | | |
|---|---|---|---|---|---|---|---|---|---|---|---|
| ANISOU | 1957 | O | LYS B | 40 | 4888 | 5122 | 4840 | 168 | 124 | −20 | O |

| | | | | | | | | | | |
|---|---|---|---|---|---|---|---|---|---|---|
| ATOM | 1958 | CB | LYS B | 40 | 15.692 | 18.623 | 41.310 | 1.00 | 42.50 | C |

| | | | | | | | | | | | |
|---|---|---|---|---|---|---|---|---|---|---|---|
| ANISOU | 1958 | CB | LYS B | 40 | 5274 | 5554 | 5320 | 36 | 107 | 36 | C |

| | | | | | | | | | | |
|---|---|---|---|---|---|---|---|---|---|---|
| ATOM | 1959 | CG | LYS B | 40 | 15.963 | 17.289 | 40.673 | 1.00 | 45.10 | C |

| | | | | | | | | | | | |
|---|---|---|---|---|---|---|---|---|---|---|---|
| ANISOU | 1959 | CG | LYS B | 40 | 5778 | 5595 | 5762 | 41 | 21 | −60 | C |

| | | | | | | | | | | |
|---|---|---|---|---|---|---|---|---|---|---|
| ATOM | 1960 | CD | LYS B | 40 | 16.599 | 17.454 | 39.290 | 1.00 | 47.24 | C |

| | | | | | | | | | | | |
|---|---|---|---|---|---|---|---|---|---|---|---|
| ANISOU | 1960 | CD | LYS B | 40 | 6045 | 6017 | 5888 | −11 | 120 | 38 | C |

| | | | | | | | | | | |
|---|---|---|---|---|---|---|---|---|---|---|
| ATOM | 1961 | CE | LYS B | 40 | 16.925 | 16.080 | 38.717 | 1.00 | 48.47 | C |

| | | | | | | | | | | | |
|---|---|---|---|---|---|---|---|---|---|---|---|
| ANISOU | 1961 | CE | LYS B | 40 | 6184 | 6072 | 6161 | 32 | 75 | −33 | C |

| | | | | | | | | | | |
|---|---|---|---|---|---|---|---|---|---|---|
| ATOM | 1962 | NZ | LYS B | 40 | 17.025 | 16.097 | 37.230 | 1.00 | 49.86 | N |

| | | | | | | | | | | | |
|---|---|---|---|---|---|---|---|---|---|---|---|
| ANISOU | 1962 | NZ | LYS B | 40 | 6345 | 6392 | 6206 | 41 | 27 | −3 | N |

| | | | | | | | | | | |
|---|---|---|---|---|---|---|---|---|---|---|
| ATOM | 1963 | N | LYS B | 41 | 14.557 | 16.882 | 43.983 | 1.00 | 40.13 | N |

| | | | | | | | | | | | |
|---|---|---|---|---|---|---|---|---|---|---|---|
| ANISOU | 1963 | N | LYS B | 41 | 5096 | 5144 | 5006 | 135 | 131 | −35 | N |

| | | | | | | | | | | |
|---|---|---|---|---|---|---|---|---|---|---|
| ATOM | 1964 | CA | LYS B | 41 | 14.899 | 16.318 | 45.286 | 1.00 | 40.89 | C |

| | | | | | | | | | | | |
|---|---|---|---|---|---|---|---|---|---|---|---|
| ANISOU | 1964 | CA | LYS B | 41 | 5280 | 5194 | 5062 | 102 | 45 | −44 | C |

| | | | | | | | | | | |
|---|---|---|---|---|---|---|---|---|---|---|
| ATOM | 1965 | C | LYS B | 41 | 16.389 | 16.230 | 45.541 | 1.00 | 40.76 | C |

| | | | | | | | | | | | |
|---|---|---|---|---|---|---|---|---|---|---|---|
| ANISOU | 1965 | C | LYS B | 41 | 5276 | 5133 | 5079 | 105 | 15 | −35 | C |

| | | | | | | | | | | |
|---|---|---|---|---|---|---|---|---|---|---|
| ATOM | 1966 | O | LYS B | 41 | 16.807 | 16.133 | 46.693 | 1.00 | 40.75 | O |

| | | | | | | | | | | | |
|---|---|---|---|---|---|---|---|---|---|---|---|
| ANISOU | 1966 | O | LYS B | 41 | 5350 | 5144 | 4991 | 235 | 82 | −100 | O |

| | | | | | | | | | | |
|---|---|---|---|---|---|---|---|---|---|---|
| ATOM | 1967 | CB | LYS B | 41 | 14.288 | 14.929 | 45.481 | 1.00 | 41.82 | C |

| | | | | | | | | | | | |
|---|---|---|---|---|---|---|---|---|---|---|---|
| ANISOU | 1967 | CB | LYS B | 41 | 5347 | 5259 | 5285 | 13 | −6 | −29 | C |

| | | | | | | | | | | |
|---|---|---|---|---|---|---|---|---|---|---|
| ATOM | 1968 | CG | LYS B | 41 | 12.773 | 14.935 | 45.556 | 1.00 | 42.86 | C |

| | | | | | | | | | | | |
|---|---|---|---|---|---|---|---|---|---|---|---|
| ANISOU | 1968 | CG | LYS B | 41 | 5413 | 5418 | 5455 | 64 | 58 | −38 | C |

| | | | | | | | | | | |
|---|---|---|---|---|---|---|---|---|---|---|
| ATOM | 1969 | CD | LYS B | 41 | 12.231 | 13.519 | 45.685 | 1.00 | 43.64 | C |

| | | | | | | | | | | | |
|---|---|---|---|---|---|---|---|---|---|---|---|
| ANISOU | 1969 | CD | LYS B | 41 | 5585 | 5463 | 5533 | 9 | 25 | 5 | C |

| | | | | | | | | | | |
|---|---|---|---|---|---|---|---|---|---|---|
| ATOM | 1970 | CE | LYS B | 41 | 10.715 | 13.496 | 45.785 | 1.00 | 44.40 | C |

| | | | | | | | | | | | |
|---|---|---|---|---|---|---|---|---|---|---|---|
| ANISOU | 1970 | CE | LYS B | 41 | 5621 | 5660 | 5588 | 7 | 23 | 23 | C |

| | | | | | | | | | | |
|---|---|---|---|---|---|---|---|---|---|---|
| ATOM | 1971 | NZ | LYS B | 41 | 10.213 | 12.102 | 45.561 | 1.00 | 45.33 | N |

| | | | | | | | | | | | |
|---|---|---|---|---|---|---|---|---|---|---|---|
| ANISOU | 1971 | HZ | LYS B | 41 | 5790 | 5727 | 5707 | −54 | 13 | 3 | N |

| | | | | | | | | | | |
|---|---|---|---|---|---|---|---|---|---|---|
| ATOM | 1972 | N | THR B | 42 | 17.233 | 16.245 | 44.517 | 1.00 | 41.12 | N |

| | | | | | | | | | | | |
|---|---|---|---|---|---|---|---|---|---|---|---|
| ANISOU | 1972 | N | THR B | 42 | 5338 | 5248 | 5039 | 103 | 18 | −9 | N |

| | | | | | | | | | | |
|---|---|---|---|---|---|---|---|---|---|---|
| ATOM | 1973 | CA | THR B | 42 | 18.675 | 16.172 | 44.614 | 1.00 | 42.23 | C |

| | | | | | | | | | | | |
|---|---|---|---|---|---|---|---|---|---|---|---|
| ANISOU | 1973 | CA | THR B | 42 | 5363 | 5471 | 5211 | 96 | 53 | −46 | C |

| | | | | | | | | | | |
|---|---|---|---|---|---|---|---|---|---|---|
| ATOM | 1974 | C | THR B | 42 | 19.353 | 17.517 | 44.488 | 1.00 | 42.63 | C |

| | | | | | | | | | | | |
|---|---|---|---|---|---|---|---|---|---|---|---|
| ANISOU | 1974 | C | THR B | 42 | 5424 | 5538 | 5235 | 64 | 45 | −9 | C |

| | | | | | | | | | | |
|---|---|---|---|---|---|---|---|---|---|---|
| ATOM | 1975 | O | THR B | 42 | 20.585 | 17.660 | 44.487 | 1.00 | 42.26 | O |

| | | | | | | | | | | | |
|---|---|---|---|---|---|---|---|---|---|---|---|
| ANISOU | 1975 | O | THR B | 42 | 5409 | 5601 | 5047 | 127 | 170 | 25 | O |

| | | | | | | | | | | |
|---|---|---|---|---|---|---|---|---|---|---|
| ATOM | 1976 | CB | THR B | 42 | 19.143 | 15.222 | 43.480 | 1.00 | 42.58 | C |

| | | | | | | | | | | | |
|---|---|---|---|---|---|---|---|---|---|---|---|
| ANISOU | 1976 | CB | THR B | 42 | 5404 | 5432 | 5342 | 104 | 47 | −84 | C |

| | | | | | | | | | | |
|---|---|---|---|---|---|---|---|---|---|---|
| ATOM | 1977 | OG1 | THR B | 42 | 18.661 | 15.746 | 42.227 | 1.00 | 43.06 | O |

| | | | | | | | | | | | |
|---|---|---|---|---|---|---|---|---|---|---|---|
| ANISOU | 1977 | OG1 | THR B | 42 | 5516 | 5531 | 5315 | 115 | 29 | −113 | O |

| | | | | | | | | | | |
|---|---|---|---|---|---|---|---|---|---|---|
| ATOM | 1978 | CG2 | THR B | 42 | 18.582 | 13.827 | 43.689 | 1.00 | 42.38 | C |

| | | | | | | | | | | | |
|---|---|---|---|---|---|---|---|---|---|---|---|
| ANISOU | 1978 | CG2 | THR B | 42 | 5284 | 5458 | 5362 | 81 | 37 | −43 | C |

| | | | | | | | | | | |
|---|---|---|---|---|---|---|---|---|---|---|
| ATOM | 1979 | N | THR B | 43 | 18.582 | 18.611 | 44.464 | 1.00 | 43.40 | N |

| | | | | | | | | | | | |
|---|---|---|---|---|---|---|---|---|---|---|---|
| ANISOU | 1979 | N | THR B | 43 | 5545 | 5622 | 5325 | 113 | 79 | −5 | N |

| | | | | | | | | | | |
|---|---|---|---|---|---|---|---|---|---|---|
| ATOM | 1980 | CA | THR B | 43 | 19.131 | 19.963 | 44.394 | 1.00 | 44.00 | C |

| | | | | | | | | | | | |
|---|---|---|---|---|---|---|---|---|---|---|---|
| ANISOU | 1980 | CA | THR B | 43 | 5568 | 5659 | 5491 | 54 | 63 | −25 | C |

| | | | | | | | | | | |
|---|---|---|---|---|---|---|---|---|---|---|
| ATOM | 1981 | C | THR B | 43 | 19.334 | 20.517 | 45.794 | 1.00 | 43.80 | C |

| | | | | | | | | | | | |
|---|---|---|---|---|---|---|---|---|---|---|---|
| ANISOU | 1981 | C | THR B | 43 | 5498 | 5638 | 5508 | 61 | 30 | −3 | C |

| | | | | | | | | | | |
|---|---|---|---|---|---|---|---|---|---|---|
| ATOM | 1982 | O | THR B | 43 | 18.456 | 20.385 | 46.642 | 1.00 | 43.49 | O |

| | | | | | | | | | | | |
|---|---|---|---|---|---|---|---|---|---|---|---|
| ANISOU | 1982 | O | THR B | 43 | 5646 | 5554 | 5325 | 129 | 49 | 53 | O |

| | | | | | | | | | | |
|---|---|---|---|---|---|---|---|---|---|---|
| ATOM | 1983 | CB | THR B | 43 | 18.139 | 20.834 | 43.595 | 1.00 | 45.06 | C |

| | | | | | | | | | | | |
|---|---|---|---|---|---|---|---|---|---|---|---|
| ANISOU | 1983 | CB | THR B | 43 | 5689 | 5745 | 5686 | 38 | −26 | 20 | C |

| | | | | | | | | | | |
|---|---|---|---|---|---|---|---|---|---|---|
| ATOM | 1984 | OG1 | THR B | 43 | 18.178 | 20.410 | 42.221 | 1.00 | 46.72 | O |

| | | | | | | | | | | | |
|---|---|---|---|---|---|---|---|---|---|---|---|
| ANISOU | 1984 | OG1 | THR B | 43 | 6001 | 6030 | 5720 | 23 | 34 | −18 | O |

| | | | | | | | | | | |
|---|---|---|---|---|---|---|---|---|---|---|
| ATOM | 1985 | CD2 | THR B | 43 | 18.425 | 22.317 | 43.673 | 1.00 | 44.21 | C |

| | | | | | | | | | | | |
|---|---|---|---|---|---|---|---|---|---|---|---|
| ANISOU | 1985 | CD2 | THR B | 43 | 5508 | 5690 | 5601 | 102 | −45 | 42 | C |

| | | | | | | | | | | |
|---|---|---|---|---|---|---|---|---|---|---|
| ATOM | 1986 | N | PRO B | 44 | 20.487 | 21.096 | 46.087 | 1.00 | 44.05 | N |

| | | | | | | | | | | | |
|---|---|---|---|---|---|---|---|---|---|---|---|
| ANISOU | 1986 | N | PRO B | 44 | 5599 | 5639 | 5501 | 20 | 7 | −14 | N |

| | | | | | | | | | | |
|---|---|---|---|---|---|---|---|---|---|---|
| ATOM | 1987 | CA | PRO B | 44 | 20.749 | 21.675 | 47.387 | 1.00 | 42.79 | C |

| | | | | | | | | | | | |
|---|---|---|---|---|---|---|---|---|---|---|---|
| ANISOU | 1987 | CA | PRO B | 44 | 5415 | 5444 | 5397 | 114 | 63 | 6 | C |

| | | | | | | | | | | |
|---|---|---|---|---|---|---|---|---|---|---|
| ATOM | 1988 | C | PRO B | 44 | 19.723 | 22.754 | 47.692 | 1.00 | 41.49 | C |

| | | | | | | | | | | | |
|---|---|---|---|---|---|---|---|---|---|---|---|
| ANISOU | 1988 | C | PRO B | 44 | 5222 | 5476 | 5068 | 64 | 74 | 39 | C |

| | | | | | | | | | | |
|---|---|---|---|---|---|---|---|---|---|---|
| ATOM | 1989 | O | PRO B | 44 | 19.317 | 23.491 | 46.786 | 1.00 | 40.84 | O |

| | | | | | | | | | | | |
|---|---|---|---|---|---|---|---|---|---|---|---|
| ANISOU | 1989 | O | PRO B | 44 | 5169 | 5295 | 5052 | 121 | 222 | 40 | O |

| | | | | | | | | | | |
|---|---|---|---|---|---|---|---|---|---|---|
| ATOM | 1990 | CB | PRO B | 44 | 22.131 | 22.311 | 47.261 | 1.00 | 43.55 | C |

| | | | | | | | | | | | |
|---|---|---|---|---|---|---|---|---|---|---|---|
| ANISOU | 1990 | CB | PRO B | 44 | 5485 | 5581 | 5480 | 12 | 22 | 37 | C |

| | | | | | | | | | | |
|---|---|---|---|---|---|---|---|---|---|---|
| ATOM | 1991 | CG | PRO B | 44 | 22.775 | 21.623 | 46.095 | 1.00 | 44.15 | C |

| | | | | | | | | | | | |
|---|---|---|---|---|---|---|---|---|---|---|---|
| ANISOU | 1991 | CG | PRO B | 44 | 5555 | 5630 | 5589 | 41 | 37 | −29 | C |

| | | | | | | | | | | |
|---|---|---|---|---|---|---|---|---|---|---|
| ATOM | 1992 | CD | PRO B | 44 | 21.652 | 21.236 | 45.164 | 1.00 | 44.16 | C |

| | | | | | | | | | | | |
|---|---|---|---|---|---|---|---|---|---|---|---|
| ANISOU | 1992 | CD | PRO B | 44 | 5583 | 5673 | 5521 | 28 | 12 | 8 | C |

| | | | | | | | | | | |
|---|---|---|---|---|---|---|---|---|---|---|
| ATOM | 1993 | N | LEU B | 45 | 19.382 | 22.927 | 48.968 | 1.00 | 39.39 | N |

| | | | | | | | | | | | |
|---|---|---|---|---|---|---|---|---|---|---|---|
| ANISOU | 1993 | N | LEU B | 45 | 4979 | 5050 | 4937 | 141 | −4 | 11 | N |

| | | | | | | | | | | |
|---|---|---|---|---|---|---|---|---|---|---|
| ATOM | 1994 | CA | LEU B | 45 | 18.420 | 23.958 | 49.355 | 1.00 | 37.24 | C |

| | | | | | | | | | | | |
|---|---|---|---|---|---|---|---|---|---|---|---|
| ANISOU | 1994 | CA | LEU B | 45 | 4669 | 4982 | 4497 | 12 | 44 | 69 | C |

| | | | | | | | | | | |
|---|---|---|---|---|---|---|---|---|---|---|
| ATOM | 1995 | C | LEU B | 45 | 19.011 | 25.341 | 49.378 | 1.00 | 37.92 | C |

| | | | | | | | | | | | |
|---|---|---|---|---|---|---|---|---|---|---|---|
| ANISOU | 1995 | C | LEU B | 45 | 4834 | 4960 | 4615 | 48 | 38 | 28 | C |

| | | | | | | | | | | |
|---|---|---|---|---|---|---|---|---|---|---|
| ATOM | 1996 | O | LEU B | 45 | 18.296 | 26.333 | 49.591 | 1.00 | 37.54 | O |

| | | | | | | | | | | | |
|---|---|---|---|---|---|---|---|---|---|---|---|
| ANISOU | 1996 | O | LEU B | 45 | 4637 | 4940 | 4685 | 22 | 55 | 117 | O |

| | | | | | | | | | | |
|---|---|---|---|---|---|---|---|---|---|---|
| ATOM | 1997 | CB | LEU B | 45 | 17.848 | 23.550 | 50.738 | 1.00 | 36.06 | C |

| | | | | | | | | | | | |
|---|---|---|---|---|---|---|---|---|---|---|---|
| ANISOU | 1997 | CB | LEU B | 45 | 4558 | 4675 | 4467 | 87 | 41 | 3 | C |

| | | | | | | | | | | |
|---|---|---|---|---|---|---|---|---|---|---|
| ATOM | 1998 | CG | LEU B | 45 | 17.069 | 22.234 | 50.710 | 1.00 | 35.04 | C |

| | | | | | | | | | | | |
|---|---|---|---|---|---|---|---|---|---|---|---|
| ANISOU | 1998 | CG | LEU B | 45 | 4356 | 4630 | 4328 | 115 | 45 | 7 | C |

| | | | | | | | | | | |
|---|---|---|---|---|---|---|---|---|---|---|
| ATOM | 1999 | CD1 | LEU B | 45 | 16.969 | 21.611 | 52.105 | 1.00 | 35.04 | C |

| | | | | | | | | | | | |
|---|---|---|---|---|---|---|---|---|---|---|---|
| ANISOU | 1999 | CD1 | LEU B | 45 | 4329 | 4589 | 4396 | 112 | 1 | 40 | C |

| | | | | | | | | | | |
|---|---|---|---|---|---|---|---|---|---|---|
| ATOM | 2000 | CD2 | LEU B | 45 | 15.652 | 22.438 | 50.176 | 1.00 | 35.12 | C |

| | | | | | | | | | | | |
|---|---|---|---|---|---|---|---|---|---|---|---|
| ANISOU | 2000 | CD2 | LEU B | 45 | 4371 | 4602 | 4372 | 98 | 18 | 72 | C |

| | | | | | | | | | | |
|---|---|---|---|---|---|---|---|---|---|---|
| ATOM | 2001 | N | ARG B | 46 | 20.324 | 25.505 | 49.161 | 1.00 | 38.69 | N |

| | | | | | | | | | | | |
|---|---|---|---|---|---|---|---|---|---|---|---|
| ANISOU | 2001 | N | ARG B | 46 | 4759 | 5158 | 4785 | 117 | 39 | −5 | N |

| | | | | | | | | | | |
|---|---|---|---|---|---|---|---|---|---|---|
| ATOM | 2002 | CA | ARG B | 46 | 20.959 | 26.808 | 49.231 | 1.00 | 40.79 | C |

| | | | | | | | | | | | |
|---|---|---|---|---|---|---|---|---|---|---|---|
| ANISOU | 2002 | CA | ARG B | 46 | 5198 | 5243 | 5058 | −27 | 37 | 46 | C |

| | | | | | | | | | | |
|---|---|---|---|---|---|---|---|---|---|---|
| ATOM | 2003 | C | ARG B | 46 | 20.201 | 27.889 | 48.481 | 1.00 | 40.42 | C |

| | | | | | | | | | | | |
|---|---|---|---|---|---|---|---|---|---|---|---|
| ANISOU | 2003 | C | ARG B | 46 | 4972 | 5268 | 5117 | −57 | 67 | 60 | C |

| | | | | | | | | | | |
|---|---|---|---|---|---|---|---|---|---|---|
| ATOM | 2004 | O | ARG B | 46 | 19.854 | 28.912 | 49.078 | 1.00 | 40.21 | O |

| | | | | | | | | | | | |
|---|---|---|---|---|---|---|---|---|---|---|---|
| ANISOU | 2004 | O | ARG B | 46 | 5052 | 5273 | 4953 | −45 | −38 | 113 | O |

| | | | | | | | | | | |
|---|---|---|---|---|---|---|---|---|---|---|
| ATOM | 2005 | CB | ARG B | 46 | 22.413 | 26.739 | 48.744 | 1.00 | 43.60 | C |

| | | | | | | | | | | | |
|---|---|---|---|---|---|---|---|---|---|---|---|
| ANISOU | 2005 | CB | ARG B | 46 | 5272 | 5788 | 5504 | 94 | 33 | 25 | C |

| | | | | | | | | | | |
|---|---|---|---|---|---|---|---|---|---|---|
| ATOM | 2006 | CG | ARG B | 46 | 23.143 | 28.074 | 48.696 | 1.00 | 47.01 | C |

| | | | | | | | | | | | |
|---|---|---|---|---|---|---|---|---|---|---|---|
| ANISOU | 2006 | CG | ARG B | 46 | 5940 | 5871 | 6050 | −59 | −84 | 38 | C |

| | | | | | | | | | | |
|---|---|---|---|---|---|---|---|---|---|---|
| ATOM | 2007 | CD | ARG B | 46 | 24.446 | 27.936 | 47.919 | 1.00 | 50.57 | C |

| | | | | | | | | | | | |
|---|---|---|---|---|---|---|---|---|---|---|---|
| ANISOU | 2007 | CD | ARG B | 46 | 6281 | 6533 | 6399 | 66 | 146 | O | C |

| | | | | | | | | | | |
|---|---|---|---|---|---|---|---|---|---|---|
| ATOM | 2008 | NE | ARG B | 46 | 25.391 | 29.034 | 48.116 | 1.00 | 53.51 | N |

| | | | | | | | | | | | |
|---|---|---|---|---|---|---|---|---|---|---|---|
| ANISOU | 2008 | NE | ARG B | 46 | 6766 | 6687 | 6879 | −100 | −6 | −47 | N |

| | | | | | | | | | | |
|---|---|---|---|---|---|---|---|---|---|---|
| ATOM | 2009 | CZ | ARG B | 46 | 26.694 | 28.973 | 47.808 | 1.00 | 55.02 | C |

| | | | | | | | | | | | |
|---|---|---|---|---|---|---|---|---|---|---|---|
| ANISOU | 2009 | CZ | ARG B | 46 | 6858 | 7009 | 7037 | −16 | 60 | −10 | C |

| | | | | | | | | | | |
|---|---|---|---|---|---|---|---|---|---|---|
| ATOM | 2010 | NH1 | ARG B | 46 | 27.223 | 27.861 | 47.288 | 1.00 | 55.70 | N |

| | | | | | | | | | | | |
|---|---|---|---|---|---|---|---|---|---|---|---|
| ANISOU | 2010 | NH1 | ARG B | 46 | 7051 | 7039 | 7073 | 36 | 68 | −9 | N |

| | | | | | | | | | | |
|---|---|---|---|---|---|---|---|---|---|---|
| ATOM | 2011 | NH2 | ARG B | 46 | 27.513 | 30.001 | 48.013 | 1.00 | 55.33 | N |

| | | | | | | | | | | | |
|---|---|---|---|---|---|---|---|---|---|---|---|
| ANISOU | 2011 | NH2 | ARG B | 46 | 6921 | 7021 | 7079 | −29 | 52 | −25 | N |

| | | | | | | | | | | |
|---|---|---|---|---|---|---|---|---|---|---|
| ATOM | 2012 | N | ARG B | 47 | 19.918 | 27.668 | 47.196 | 1.00 | 40.88 | N |

| | | | | | | | | | | | |
|---|---|---|---|---|---|---|---|---|---|---|---|
| ANISOU | 2012 | N | ARG B | 47 | 5065 | 5326 | 5141 | −24 | 49 | 53 | N |

| | | | | | | | | | | |
|---|---|---|---|---|---|---|---|---|---|---|
| ATOM | 2013 | CA | ARG B | 47 | 19.268 | 28.683 | 46.366 | 1.00 | 41.72 | C |

| | | | | | | | | | | | |
|---|---|---|---|---|---|---|---|---|---|---|---|
| ANISOU | 2013 | CA | ARG B | 47 | 5157 | 5397 | 5298 | 61 | 2 | 63 | C |

| | | | | | | | | | | |
|---|---|---|---|---|---|---|---|---|---|---|
| ATOM | 2014 | C | ARG B | 47 | 17.910 | 29.080 | 46.925 | 1.00 | 40.16 | C |

| | | | | | | | | | | | |
|---|---|---|---|---|---|---|---|---|---|---|---|
| ANISOU | 2014 | C | ARG B | 47 | 5061 | 5228 | 4970 | −33 | −47 | 70 | C |

| | | | | | | | | | | |
|---|---|---|---|---|---|---|---|---|---|---|
| ATOM | 2015 | O | ARG B | 47 | 17.553 | 30.253 | 47.068 | 1.00 | 40.12 | O |

| | | | | | | | | | | | |
|---|---|---|---|---|---|---|---|---|---|---|---|
| ANISOU | 2015 | O | ARG B | 47 | 4984 | 5230 | 5032 | −53 | −26 | 146 | O |

| | | | | | | | | | | |
|---|---|---|---|---|---|---|---|---|---|---|
| ATOM | 2016 | CB | ARG B | 47 | 19.150 | 28.197 | 44.918 | 1.00 | 44.11 | C |

| | | | | | | | | | | | |
|---|---|---|---|---|---|---|---|---|---|---|---|
| ANISOU | 2016 | CB | ARG B | 47 | 5675 | 5648 | 5438 | −10 | −30 | −33 | C |

| | | | | | | | | | | |
|---|---|---|---|---|---|---|---|---|---|---|
| ATOM | 2017 | CG | ARG B | 47 | 20.485 | 28.035 | 44.191 | 1.00 | 46.48 | C |

| | | | | | | | | | | | |
|---|---|---|---|---|---|---|---|---|---|---|---|
| ANISOU | 2017 | CG | ARG B | 47 | 5763 | 6055 | 5843 | 24 | 48 | −46 | C |

| | | | | | | | | | | |
|---|---|---|---|---|---|---|---|---|---|---|
| ATOM | 2018 | CD | ARG B | 47 | 20.918 | 29.350 | 43.566 | 1.00 | 49.08 | C |

| | | | | | | | | | | | |
|---|---|---|---|---|---|---|---|---|---|---|---|
| ANISOU | 2018 | CD | ARG B | 47 | 6238 | 6175 | 6236 | −46 | 95 | 60 | C |

| | | | | | | | | | | |
|---|---|---|---|---|---|---|---|---|---|---|
| ATOM | 2019 | NE | ARG B | 47 | 20.086 | 29.649 | 42.391 | 1.00 | 51.08 | N |

| | | | | | | | | | | | |
|---|---|---|---|---|---|---|---|---|---|---|---|
| ANISOU | 2019 | NE | ARG B | 47 | 6421 | 6599 | 6386 | −15 | −45 | −18 | N |

| | | | | | | | | | | |
|---|---|---|---|---|---|---|---|---|---|---|
| ATOM | 2020 | CZ | ARG B | 47 | 20.035 | 30.869 | 41.858 | 1.00 | 52.78 | C |

| | | | | | | | | | | | |
|---|---|---|---|---|---|---|---|---|---|---|---|
| ANISOU | 2020 | CZ | ARG B | 47 | 6701 | 6658 | 6694 | −20 | 6 | 38 | C |

| | | | | | | | | | | |
|---|---|---|---|---|---|---|---|---|---|---|
| ATOM | 2021 | NH1 | ARG B | 47 | 20.751 | 31.839 | 42.427 | 1.00 | 53.44 | N |

| | | | | | | | | | | | |
|---|---|---|---|---|---|---|---|---|---|---|---|
| ANISOU | 2021 | NH1 | ARG B | 47 | 6795 | 6747 | 6764 | −80 | −8 | −6 | N |

| | | | | | | | | | | |
|---|---|---|---|---|---|---|---|---|---|---|
| ATOM | 2022 | NH2 | ARG B | 47 | 19.288 | 31.158 | 40.793 | 1.00 | 53.59 | N |

| | | | | | | | | | | | |
|---|---|---|---|---|---|---|---|---|---|---|---|
| ANISOU | 2022 | NH2 | ARG B | 47 | 6751 | 6903 | 6707 | −38 | −11 | 27 | N |

| | | | | | | | | | | |
|---|---|---|---|---|---|---|---|---|---|---|
| ATOM | 2023 | N | LEU B | 48 | 17.163 | 28.069 | 47.338 | 1.00 | 39.03 | N |

| | | | | | | | | | | | |
|---|---|---|---|---|---|---|---|---|---|---|---|
| ANISOU | 2023 | N | LEU B | 48 | 4892 | 5095 | 4843 | 59 | −62 | −1 | N |

| | | | | | | | | | | |
|---|---|---|---|---|---|---|---|---|---|---|
| ATOM | 2024 | CA | LEU B | 48 | 15.844 | 28.266 | 47.966 | 1.00 | 38.40 | C |

| | | | | | | | | | | | |
|---|---|---|---|---|---|---|---|---|---|---|---|
| ANISOU | 2024 | CA | LEU B | 48 | 4919 | 4986 | 4684 | 3 | −39 | 4 | C |

| | | | | | | | | | | |
|---|---|---|---|---|---|---|---|---|---|---|
| ATOM | 2025 | C | LEU B | 48 | 15.929 | 29.136 | 49.206 | 1.00 | 35.83 | C |

| | | | | | | | | | | | |
|---|---|---|---|---|---|---|---|---|---|---|---|
| ANISOU | 2025 | C | LEU B | 48 | 4434 | 4674 | 4505 | 72 | 25 | 129 | C |

| | | | | | | | | | | |
|---|---|---|---|---|---|---|---|---|---|---|
| ATOM | 2026 | O | LEU B | 48 | 15.225 | 30.127 | 49.423 | 1.00 | 33.87 | O |

| | | | | | | | | | | | |
|---|---|---|---|---|---|---|---|---|---|---|---|
| ANISOU | 2026 | O | LEU B | 48 | 4272 | 4582 | 4015 | −30 | 149 | 170 | O |

| | | | | | | | | | | |
|---|---|---|---|---|---|---|---|---|---|---|
| ATOM | 2027 | CB | LEU B | 48 | 15.423 | 26.831 | 48.250 | 1.00 | 40.15 | C |

| | | | | | | | | | | | |
|---|---|---|---|---|---|---|---|---|---|---|---|
| ANISOU | 2027 | CB | LEU B | 48 | 5145 | 5048 | 5061 | −62 | −38 | 7 | C |

| | | | | | | | | | | |
|---|---|---|---|---|---|---|---|---|---|---|
| ATOM | 2028 | CG | LEU B | 48 | 14.111 | 26.471 | 48.907 | 1.00 | 40.86 | C |

| | | | | | | | | | | | |
|---|---|---|---|---|---|---|---|---|---|---|---|
| ANISOU | 2028 | CG | LEU B | 48 | 5187 | 5210 | 5127 | −14 | −3 | 7 | C |

| | | | | | | | | | | |
|---|---|---|---|---|---|---|---|---|---|---|
| ATOM | 2029 | CD1 | LEU B | 48 | 13.755 | 25.033 | 48.540 | 1.00 | 40.98 | C |

| | | | | | | | | | | | |
|---|---|---|---|---|---|---|---|---|---|---|---|
| ANISOU | 2029 | CD1 | LEU B | 48 | 5215 | 5184 | 5173 | −5 | −20 | 18 | C |

| | | | | | | | | | | |
|---|---|---|---|---|---|---|---|---|---|---|
| ATOM | 2030 | CD2 | LEU B | 48 | 14.216 | 26.648 | 50.412 | 1.00 | 40.80 | C |

| | | | | | | | | | | | |
|---|---|---|---|---|---|---|---|---|---|---|---|
| ANISOU | 2030 | CD2 | LEU B | 48 | 5218 | 5156 | 5129 | 2 | −1 | 11 | C |

| | | | | | | | | | | |
|---|---|---|---|---|---|---|---|---|---|---|
| ATOM | 2031 | N | MET B | 49 | 16.874 | 28.814 | 50.092 | 1.00 | 34.39 | N |

| | | | | | | | | | | | |
|---|---|---|---|---|---|---|---|---|---|---|---|
| ANISOU | 2031 | N | MET B | 49 | 4319 | 4518 | 4228 | 8 | 119 | 80 | N |

| | | | | | | | | | | |
|---|---|---|---|---|---|---|---|---|---|---|
| ATOM | 2032 | CA | MET B | 49 | 17.116 | 29.539 | 51.333 | 1.00 | 34.12 | C |

| | | | | | | | | | | | |
|---|---|---|---|---|---|---|---|---|---|---|---|
| ANISOU | 2032 | CA | MET B | 49 | 4276 | 4389 | 4299 | −3 | 51 | 69 | C |

| | | | | | | | | | | |
|---|---|---|---|---|---|---|---|---|---|---|
| ATOM | 2033 | C | MET B | 49 | 17.583 | 30.969 | 51.101 | 1.00 | 34.71 | C |

| | | | | | | | | | | | |
|---|---|---|---|---|---|---|---|---|---|---|---|
| ANISOU | 2033 | C | MET B | 49 | 4408 | 4407 | 4372 | −6 | −55 | 88 | C |

| | | | | | | | | | | |
|---|---|---|---|---|---|---|---|---|---|---|
| ATOM | 2034 | O | MET B | 49 | 17.141 | 31.906 | 51.768 | 1.00 | 35.24 | O |

| | | | | | | | | | | | |
|---|---|---|---|---|---|---|---|---|---|---|---|
| ANISOU | 2034 | O | MET B | 49 | 4471 | 4591 | 4326 | −68 | −36 | 10 | O |

| | | | | | | | | | | |
|---|---|---|---|---|---|---|---|---|---|---|
| ATOM | 2035 | CB | MET B | 49 | 18.151 | 28.776 | 52.170 | 1.00 | 33.93 | C |

| | | | | | | | | | | | |
|---|---|---|---|---|---|---|---|---|---|---|---|
| ANISOU | 2035 | CB | MET B | 49 | 4280 | 4423 | 4188 | −17 | 33 | 32 | C |

| | | | | | | | | | | |
|---|---|---|---|---|---|---|---|---|---|---|
| ATOM | 2036 | CG | MET B | 49 | 17.684 | 27.370 | 52.538 | 1.00 | 34.25 | C |

| | | | | | | | | | | | |
|---|---|---|---|---|---|---|---|---|---|---|---|
| ANISOU | 2036 | CG | MET B | 49 | 4399 | 4404 | 4211 | 7 | 28 | 64 | C |

| | | | | | | | | | | |
|---|---|---|---|---|---|---|---|---|---|---|
| ATOM | 2037 | SD | MET B | 49 | 19.052 | 26.281 | 53.024 | 1.00 | 34.57 | S |

| | | | | | | | | | | | |
|---|---|---|---|---|---|---|---|---|---|---|---|
| ANISOU | 2037 | SD | MET B | 49 | 4685 | 4519 | 3929 | 117 | −48 | 74 | S |

| | | | | | | | | | | |
|---|---|---|---|---|---|---|---|---|---|---|
| ATOM | 2038 | CE | MET B | 49 | 19.657 | 27.098 | 54.499 | 1.00 | 33.51 | C |

| | | | | | | | | | | | |
|---|---|---|---|---|---|---|---|---|---|---|---|
| ANISOU | 2038 | CE | MET B | 49 | 4492 | 4149 | 4091 | 7 | −7 | 53 | C |

| | | | | | | | | | | |
|---|---|---|---|---|---|---|---|---|---|---|
| ATOM | 2039 | N | GLU B | 50 | 18.483 | 31.134 | 50.133 | 1.00 | 35.37 | N |

| | | | | | | | | | | | |
|---|---|---|---|---|---|---|---|---|---|---|---|
| ANISOU | 2039 | N | GLU B | 50 | 4373 | 4612 | 4455 | −5 | −43 | 48 | N |

| | | | | | | | | | | |
|---|---|---|---|---|---|---|---|---|---|---|
| ATOM | 2040 | CA | GLU B | 50 | 18.973 | 32.483 | 49.819 | 1.00 | 37.33 | C |

| | | | | | | | | | | | |
|---|---|---|---|---|---|---|---|---|---|---|---|
| ANISOU | 2040 | CA | GLU B | 50 | 4760 | 4685 | 4738 | −32 | 39 | 142 | C |

| | | | | | | | | | | |
|---|---|---|---|---|---|---|---|---|---|---|
| ATOM | 2041 | C | GLU B | 50 | 17.862 | 33.318 | 49.214 | 1.00 | 36.13 | C |

| | | | | | | | | | | | |
|---|---|---|---|---|---|---|---|---|---|---|---|
| ANISOU | 2041 | C | GLU B | 50 | 4613 | 4705 | 4408 | −96 | 126 | 158 | C |

| | | | | | | | | | | |
|---|---|---|---|---|---|---|---|---|---|---|
| ATOM | 2042 | O | GLU B | 50 | 17.652 | 34.472 | 49.616 | 1.00 | 36.27 | O |

| | | | | | | | | | | | |
|---|---|---|---|---|---|---|---|---|---|---|---|
| ANISOU | 2042 | O | GLU B | 50 | 4673 | 4633 | 4475 | −69 | 30 | 282 | O |

| | | | | | | | | | | |
|---|---|---|---|---|---|---|---|---|---|---|
| ATOM | 2043 | CB | GLU B | 50 | 20.220 | 32.371 | 48.938 | 1.00 | 40.63 | C |

| | | | | | | | | | | | |
|---|---|---|---|---|---|---|---|---|---|---|---|
| ANISOU | 2043 | CB | GLU B | 50 | 5035 | 5254 | 5147 | −23 | 223 | 27 | C |

| | | | | | | | | | | |
|---|---|---|---|---|---|---|---|---|---|---|
| ATOM | 2044 | CG | GLU B | 50 | 21.395 | 31.710 | 49.659 | 1.00 | 43.80 | C |

| | | | | | | | | | | | |
|---|---|---|---|---|---|---|---|---|---|---|---|
| ANISOU | 2044 | CG | GLU B | 50 | 5464 | 5582 | 5594 | 62 | −106 | 117 | C |

| | | | | | | | | | | |
|---|---|---|---|---|---|---|---|---|---|---|
| ATOM | 2045 | CD | GLU B | 50 | 22.628 | 31.649 | 48.780 | 1.00 | 46.59 | C |

| | | | | | | | | | | | |
|---|---|---|---|---|---|---|---|---|---|---|---|
| ANISOU | 2045 | CD | GLU B | 50 | 5800 | 6013 | 5887 | 62 | 103 | 17 | C |

| | | | | | | | | | | |
|---|---|---|---|---|---|---|---|---|---|---|
| ATOM | 2046 | OE1 | GLU B | 50 | 22.525 | 32.069 | 47.599 | 1.00 | 48.10 | O |

| | | | | | | | | | | | |
|---|---|---|---|---|---|---|---|---|---|---|---|
| ANISOU | 2046 | OE1 | GLU B | 50 | 6083 | 6229 | 5963 | 98 | 32 | 65 | O |

| | | | | | | | | | | |
|---|---|---|---|---|---|---|---|---|---|---|
| ATOM | 2047 | OE2 | GLU B | 50 | 23.706 | 31.205 | 49.241 | 1.00 | 47.29 | O |

| | | | | | | | | | | | |
|---|---|---|---|---|---|---|---|---|---|---|---|
| ANISOU | 2047 | OE2 | GLU B | 50 | 5930 | 6049 | 5988 | 96 | −1 | 66 | O |

| | | | | | | | | | | |
|---|---|---|---|---|---|---|---|---|---|---|
| ATOM | 2048 | N | ALA B | 51 | 17.014 | 32.728 | 48.391 | 1.00 | 36.59 | N |

| | | | | | | | | | | | |
|---|---|---|---|---|---|---|---|---|---|---|---|
| ANISOU | 2048 | N | ALA B | 51 | 4741 | 4700 | 4461 | −48 | 100 | 92 | N |

| | | | | | | | | | | |
|---|---|---|---|---|---|---|---|---|---|---|
| ATOM | 2049 | CA | ALA B | 51 | 15.878 | 33.424 | 47.783 | 1.00 | 36.88 | C |

| | | | | | | | | | | | |
|---|---|---|---|---|---|---|---|---|---|---|---|
| ANISOU | 2049 | CA | ALA B | 51 | 4770 | 4707 | 4537 | −17 | 34 | 75 | C |

| | | | | | | | | | | |
|---|---|---|---|---|---|---|---|---|---|---|
| ATOM | 2050 | C | ALA B | 51 | 14.925 | 33.921 | 48.856 | 1.00 | 37.40 | C |

| | | | | | | | | | | | |
|---|---|---|---|---|---|---|---|---|---|---|---|
| ANISOU | 2050 | C | ALA B | 51 | 4718 | 4807 | 4687 | 0 | 88 | 107 | C |

| | | | | | | | | | | |
|---|---|---|---|---|---|---|---|---|---|---|
| ATOM | 2051 | O | ALA B | 51 | 14.507 | 35.084 | 48.874 | 1.00 | 37.53 | O |

| | | | | | | | | | | | |
|---|---|---|---|---|---|---|---|---|---|---|---|
| ANISOU | 2051 | O | ALA B | 51 | 4832 | 4823 | 4606 | −26 | 115 | 218 | O |

| | | | | | | | | | | |
|---|---|---|---|---|---|---|---|---|---|---|
| ATOM | 2052 | CB | ALA B | 51 | 15.153 | 32.517 | 46.806 | 1.00 | 37.13 | C |

| | | | | | | | | | | | |
|---|---|---|---|---|---|---|---|---|---|---|---|
| ANISOU | 2052 | CB | ALA B | 51 | 4688 | 4770 | 4650 | −54 | 5 | 78 | C |

| | | | | | | | | | | |
|---|---|---|---|---|---|---|---|---|---|---|
| ATOM | 2053 | N | PHE B | 52 | 14.601 | 33.017 | 49.800 | 1.00 | 37.58 | N |

| | | | | | | | | | | | |
|---|---|---|---|---|---|---|---|---|---|---|---|
| ANISOU | 2053 | N | PHE B | 52 | 4743 | 4831 | 4705 | 26 | 62 | 139 | N |

| | | | | | | | | | | |
|---|---|---|---|---|---|---|---|---|---|---|
| ATOM | 2054 | CA | PHE B | 52 | 13.743 | 33.419 | 50.902 | 1.00 | 36.93 | C |

| | | | | | | | | | | | |
|---|---|---|---|---|---|---|---|---|---|---|---|
| ANISOU | 2054 | CA | PHE B | 52 | 4690 | 4678 | 4664 | 30 | 40 | 154 | C |

| | | | | | | | | | | |
|---|---|---|---|---|---|---|---|---|---|---|
| ATOM | 2055 | C | PHE B | 52 | 14.340 | 34.569 | 51.694 | 1.00 | 37.67 | C |

| | | | | | | | | | | | |
|---|---|---|---|---|---|---|---|---|---|---|---|
| ANISOU | 2055 | C | PHE B | 52 | 4818 | 4750 | 4744 | −48 | 48 | 146 | C |

| | | | | | | | | | | |
|---|---|---|---|---|---|---|---|---|---|---|
| ATOM | 2056 | O | PHE B | 52 | 13.624 | 35.538 | 51.975 | 1.00 | 38.33 | O |

| | | | | | | | | | | | |
|---|---|---|---|---|---|---|---|---|---|---|---|
| ANISOU | 2056 | O | PHE B | 52 | 4861 | 4778 | 4924 | 11 | 13 | 161 | O |

| | | | | | | | | | | |
|---|---|---|---|---|---|---|---|---|---|---|
| ATOM | 2057 | CB | PHE B | 52 | 13.465 | 32.237 | 51.841 | 1.00 | 35.85 | C |

| | | | | | | | | | | | |
|---|---|---|---|---|---|---|---|---|---|---|---|
| ANISOU | 2057 | CB | PHE B | 52 | 4469 | 4592 | 4561 | −1 | 42 | 52 | C |

| | | | | | | | | | | |
|---|---|---|---|---|---|---|---|---|---|---|
| ATOM | 2058 | CG | PHE B | 52 | 12.426 | 32.613 | 52.870 | 1.00 | 35.18 | C |

| | | | | | | | | | | | |
|---|---|---|---|---|---|---|---|---|---|---|---|
| ANISOU | 2058 | CG | PHE B | 52 | 4457 | 4478 | 4431 | −25 | −1 | 28 | C |

| | | | | | | | | | | |
|---|---|---|---|---|---|---|---|---|---|---|
| ATOM | 2059 | CD1 | PHE B | 52 | 11.084 | 32.470 | 52.572 | 1.00 | 35.08 | C |

| | | | | | | | | | | | |
|---|---|---|---|---|---|---|---|---|---|---|---|
| ANISOU | 2059 | CD1 | PHE B | 52 | 4447 | 4503 | 4380 | 7 | −25 | 29 | C |

| | | | | | | | | | | |
|---|---|---|---|---|---|---|---|---|---|---|
| ATOM | 2060 | CD2 | PHE B | 52 | 12.780 | 33.118 | 54.107 | 1.00 | 34.44 | C |

| | | | | | | | | | | | |
|---|---|---|---|---|---|---|---|---|---|---|---|
| ANISOU | 2060 | CD2 | PHE B | 52 | 4289 | 4369 | 4427 | −32 | −34 | 47 | C |

| | | | | | | | | | | |
|---|---|---|---|---|---|---|---|---|---|---|
| ATOM | 2061 | CE1 | PHE B | 52 | 10.112 | 32.806 | 53.487 | 1.00 | 34.97 | C |

| | | | | | | | | | | | |
|---|---|---|---|---|---|---|---|---|---|---|---|
| ANISOU | 2061 | CE1 | PHE B | 52 | 4508 | 4384 | 4394 | 13 | 5 | 26 | C |

| | | | | | | | | | | |
|---|---|---|---|---|---|---|---|---|---|---|
| ATOM | 2062 | CE2 | PHE B | 52 | 11.797 | 33.479 | 55.018 | 1.00 | 34.48 | C |

| | | | | | | | | | | | |
|---|---|---|---|---|---|---|---|---|---|---|---|
| ANISOU | 2062 | CE2 | PHE B | 52 | 4399 | 4372 | 4328 | −28 | −33 | 46 | C |

| | | | | | | | | | | |
|---|---|---|---|---|---|---|---|---|---|---|
| ATOM | 2063 | CZ | PHE B | 52 | 10.467 | 33.323 | 54.709 | 1.00 | 34.37 | C |

| | | | | | | | | | | | |
|---|---|---|---|---|---|---|---|---|---|---|---|
| ANISOU | 2063 | CZ | PHE B | 52 | 4434 | 4265 | 4361 | −14 | −1 | 92 | C |

| | | | | | | | | | | |
|---|---|---|---|---|---|---|---|---|---|---|
| ATOM | 2064 | N | ALA B | 53 | 15.604 | 34.495 | 52.090 | 1.00 | 38.42 | N |

| | | | | | | | | | | | |
|---|---|---|---|---|---|---|---|---|---|---|---|
| ANISOU | 2064 | N | ALA B | 53 | 4863 | 4808 | 4926 | 32 | 12 | 237 | N |

| | | | | | | | | | | |
|---|---|---|---|---|---|---|---|---|---|---|
| ATOM | 2065 | CA | ALA B | 53 | 16.237 | 35.536 | 52.894 | 1.00 | 39.93 | C |

| | | | | | | | | | | | |
|---|---|---|---|---|---|---|---|---|---|---|---|
| ANISOU | 2065 | CA | ALA B | 53 | 5008 | 5046 | 5119 | −34 | 19 | 72 | C |

| | | | | | | | | | | |
|---|---|---|---|---|---|---|---|---|---|---|
| ATOM | 2066 | C | ALA B | 53 | 16.258 | 36.900 | 52.208 | 1.00 | 41.09 | C |

| | | | | | | | | | | | |
|---|---|---|---|---|---|---|---|---|---|---|---|
| ANISOU | 2066 | C | ALA B | 53 | 5200 | 5056 | 5356 | −12 | 16 | 88 | C |

| | | | | | | | | | | |
|---|---|---|---|---|---|---|---|---|---|---|
| ATOM | 2067 | O | ALA B | 53 | 15.906 | 37.943 | 52.769 | 1.00 | 40.06 | O |

| | | | | | | | | | | | |
|---|---|---|---|---|---|---|---|---|---|---|---|
| ANISOU | 2067 | O | ALA B | 53 | 5088 | 4979 | 5154 | −216 | 148 | 133 | O |

| | | | | | | | | | | |
|---|---|---|---|---|---|---|---|---|---|---|
| ATOM | 2068 | CB | ALA B | 53 | 17.677 | 35.102 | 53.183 | 1.00 | 40.13 | C |

| | | | | | | | | | | | |
|---|---|---|---|---|---|---|---|---|---|---|---|
| ANISOU | 2068 | CB | ALA B | 53 | 5088 | 5003 | 5155 | 11 | −30 | 134 | C |

| | | | | | | | | | | |
|---|---|---|---|---|---|---|---|---|---|---|
| ATOM | 2069 | N | LYS B | 54 | 16.643 | 36.878 | 50.936 | 1.00 | 43.57 | N |

| | | | | | | | | | | | |
|---|---|---|---|---|---|---|---|---|---|---|---|
| ANISOU | 2069 | N | LYS B | 54 | 5528 | 5564 | 5465 | −12 | 77 | 36 | N |

| | | | | | | | | | | |
|---|---|---|---|---|---|---|---|---|---|---|
| ATOM | 2070 | CA | LYS B | 54 | 16.728 | 38.069 | 50.089 | 1.00 | 45.45 | C |

| | | | | | | | | | | | |
|---|---|---|---|---|---|---|---|---|---|---|---|
| ANISOU | 2070 | CA | LYS B | 54 | 5911 | 5644 | 5712 | −37 | 19 | 96 | C |

| | | | | | | | | | | |
|---|---|---|---|---|---|---|---|---|---|---|
| ATOM | 2071 | C | LYS B | 54 | 15.370 | 38.753 | 50.048 | 1.00 | 47.50 | C |

| | | | | | | | | | | | |
|---|---|---|---|---|---|---|---|---|---|---|---|
| ANISOU | 2071 | C | LYS B | 54 | 6043 | 6021 | 5983 | 98 | 25 | 77 | C |

| | | | | | | | | | | |
|---|---|---|---|---|---|---|---|---|---|---|
| ATOM | 2072 | O | LYS B | 54 | 15.272 | 39.951 | 50.325 | 1.00 | 47.24 | O |

| | | | | | | | | | | | |
|---|---|---|---|---|---|---|---|---|---|---|---|
| ANISOU | 2072 | O | LYS B | 54 | 5943 | 6011 | 5997 | 33 | 6 | 76 | O |

| | | | | | | | | | | |
|---|---|---|---|---|---|---|---|---|---|---|
| ATOM | 2073 | CB | LYS B | 54 | 17.191 | 37.688 | 48.700 | 1.00 | 45.58 | C |

| | | | | | | | | | | | |
|---|---|---|---|---|---|---|---|---|---|---|---|
| ANISOU | 2073 | CB | LYS B | 54 | 5829 | 5754 | 5737 | −27 | 43 | 75 | C |

| | | | | | | | | | | |
|---|---|---|---|---|---|---|---|---|---|---|
| ATOM | 2074 | N | ARG B | 55 | 14.302 | 37.984 | 49.836 | 1.00 | 49.68 | N |

| | | | | | | | | | | | |
|---|---|---|---|---|---|---|---|---|---|---|---|
| ANISOU | 2074 | N | ARG B | 55 | 6363 | 6251 | 6261 | −87 | −40 | 45 | N |

| | | | | | | | | | | |
|---|---|---|---|---|---|---|---|---|---|---|
| ATOM | 2075 | CA | ARG B | 55 | 12.965 | 38.572 | 49.877 | 1.00 | 51.93 | C |

| | | | | | | | | | | | |
|---|---|---|---|---|---|---|---|---|---|---|---|
| ANISOU | 2075 | CA | ARG B | 55 | 6546 | 6594 | 6589 | 90 | 13 | 57 | C |

| | | | | | | | | | | |
|---|---|---|---|---|---|---|---|---|---|---|
| ATOM | 2076 | C | ARG B | 55 | 12.618 | 39.243 | 51.196 | 1.00 | 52.04 | C |

| | | | | | | | | | | | |
|---|---|---|---|---|---|---|---|---|---|---|---|
| ANISOU | 2076 | C | ARG B | 55 | 6571 | 6574 | 6625 | −10 | 30 | 23 | C |

| | | | | | | | | | | |
|---|---|---|---|---|---|---|---|---|---|---|
| ATOM | 2077 | O | ARG B | 55 | 11.855 | 40.222 | 51.179 | 1.00 | 51.46 | O |

| | | | | | | | | | | | |
|---|---|---|---|---|---|---|---|---|---|---|---|
| ANISOU | 2077 | O | ARG B | 55 | 6615 | 6515 | 6424 | 1 | 10 | 63 | O |

| | | | | | | | | | | |
|---|---|---|---|---|---|---|---|---|---|---|
| ATOM | 2078 | CB | ARG B | 55 | 11.887 | 37.564 | 49.499 | 1.00 | 54.25 | C |

| | | | | | | | | | | | |
|---|---|---|---|---|---|---|---|---|---|---|---|
| ANISOU | 2078 | CB | ARG B | 55 | 6811 | 6778 | 7022 | −70 | 25 | 11 | C |

| | | | | | | | | | | |
|---|---|---|---|---|---|---|---|---|---|---|
| ATOM | 2079 | CG | ARG B | 55 | 11.502 | 37.719 | 48.028 | 1.00 | 57.16 | C |

| | | | | | | | | | | | |
|---|---|---|---|---|---|---|---|---|---|---|---|
| ANISOU | 2079 | CG | ARG B | 55 | 7336 | 7253 | 7127 | −23 | −30 | 20 | C |

| | | | | | | | | | | |
|---|---|---|---|---|---|---|---|---|---|---|
| ATOM | 2080 | CD | ARG B | 55 | 9.984 | 37.592 | 47.893 | 1.00 | 59.38 | C |

| | | | | | | | | | | | |
|---|---|---|---|---|---|---|---|---|---|---|---|
| ANISOU | 2080 | CD | ARG B | 55 | 7398 | 7582 | 7583 | −3 | −5 | 61 | C |

| | | | | | | | | | | |
|---|---|---|---|---|---|---|---|---|---|---|
| ATOM | 2081 | NE | ARG B | 55 | 9.710 | 36.806 | 46.692 | 1.00 | 61.03 | N |

| | | | | | | | | | | | |
|---|---|---|---|---|---|---|---|---|---|---|---|
| ANISOU | 2081 | NE | ARG B | 55 | 7771 | 7742 | 7674 | 12 | −24 | −10 | N |

| | | | | | | | | | | |
|---|---|---|---|---|---|---|---|---|---|---|
| ATOM | 2082 | CZ | ARG B | 55 | 8.543 | 36.267 | 46.382 | 1.00 | 62.13 | C |

| | | | | | | | | | | | |
|---|---|---|---|---|---|---|---|---|---|---|---|
| ANISOU | 2082 | CZ | ARG B | 55 | 7830 | 7885 | 7892 | −34 | −52 | 26 | C |

| | | | | | | | | | | |
|---|---|---|---|---|---|---|---|---|---|---|
| ATOM | 2083 | NH1 | ARG B | 55 | 7.503 | 36.433 | 47.191 | 1.00 | 62.74 | N |

| | | | | | | | | | | | |
|---|---|---|---|---|---|---|---|---|---|---|---|
| ANISOU | 2083 | NH1 | ARG B | 55 | 7936 | 7955 | 7948 | 24 | −5 | 19 | N |

| | | | | | | | | | | |
|---|---|---|---|---|---|---|---|---|---|---|
| ATOM | 2084 | NH2 | ARG B | 55 | 8.461 | 35.570 | 45.256 | 1.00 | 62.58 | N |

| | | | | | | | | | | | |
|---|---|---|---|---|---|---|---|---|---|---|---|
| ANISOU | 2084 | NH2 | ARG B | 55 | 7907 | 7940 | 7929 | −12 | 2 | −10 | N |

| | | | | | | | | | | |
|---|---|---|---|---|---|---|---|---|---|---|
| ATOM | 2085 | N | GLN B | 56 | 13.149 | 38.796 | 52.333 | 1.00 | 52.31 | N |

| | | | | | | | | | | | |
|---|---|---|---|---|---|---|---|---|---|---|---|
| ANISOU | 2085 | N | GLN B | 56 | 6603 | 6578 | 6693 | 46 | 0 | 58 | N |

| | | | | | | | | | | |
|---|---|---|---|---|---|---|---|---|---|---|
| ATOM | 2086 | CA | GLN B | 56 | 12.893 | 39.457 | 53.600 | 1.00 | 53.36 | C |

| | | | | | | | | | | | |
|---|---|---|---|---|---|---|---|---|---|---|---|
| ANISOU | 2086 | CA | GLN B | 56 | 6781 | 6761 | 6734 | 53 | −13 | 16 | C |

| | | | | | | | | | | |
|---|---|---|---|---|---|---|---|---|---|---|
| ATOM | 2087 | C | GLN B | 56 | 13.927 | 40.547 | 53.850 | 1.00 | 53.81 | C |

| | | | | | | | | | | | |
|---|---|---|---|---|---|---|---|---|---|---|---|
| ANISOU | 2087 | C | GLN B | 56 | 6817 | 6773 | 6855 | 47 | −41 | 44 | C |

| | | | | | | | | | | |
|---|---|---|---|---|---|---|---|---|---|---|
| ATOM | 2088 | O | GLN B | 56 | 13.934 | 41.194 | 54.894 | 1.00 | 54.10 | O |

| | | | | | | | | | | | |
|---|---|---|---|---|---|---|---|---|---|---|---|
| ANISOU | 2088 | O | GLN B | 56 | 6871 | 6810 | 6874 | 33 | −32 | 32 | O |

| | | | | | | | | | | |
|---|---|---|---|---|---|---|---|---|---|---|
| ATOM | 2089 | CB | GLN B | 56 | 12.924 | 38.482 | 54.781 | 1.00 | 53.69 | C |

| | | | | | | | | | | | |
|---|---|---|---|---|---|---|---|---|---|---|---|
| ANISOU | 2089 | CB | GLN B | 56 | 6799 | 6802 | 6799 | 44 | −3 | 50 | C |

| | | | | | | | | | | |
|---|---|---|---|---|---|---|---|---|---|---|
| ATOM | 2090 | CG | GLN B | 56 | 12.304 | 37.127 | 54.536 | 1.00 | 54.05 | C |

| | | | | | | | | | | | |
|---|---|---|---|---|---|---|---|---|---|---|---|
| ANISOU | 2090 | CG | GLN B | 56 | 6863 | 6836 | 6838 | −1 | 21 | 23 | C |

| | | | | | | | | | | |
|---|---|---|---|---|---|---|---|---|---|---|
| ATOM | 2091 | CD | GLN B | 56 | 10.941 | 37.214 | 53.878 | 1.00 | 54.55 | C |

| | | | | | | | | | | | |
|---|---|---|---|---|---|---|---|---|---|---|---|
| ANISOU | 2091 | CD | GLN B | 56 | 6915 | 6923 | 6888 | 5 | −8 | 24 | C |

| | | | | | | | | | | |
|---|---|---|---|---|---|---|---|---|---|---|
| ATOM | 2092 | OE1 | GLN B | 56 | 10.150 | 38.109 | 54.195 | 1.00 | 55.10 | O |

| | | | | | | | | | | | |
|---|---|---|---|---|---|---|---|---|---|---|---|
| ANISOU | 2092 | OE1 | GLN B | 58 | 6998 | 6968 | 6969 | 38 | 4 | 1 | O |

| | | | | | | | | | | |
|---|---|---|---|---|---|---|---|---|---|---|
| ATOM | 2093 | NE2 | GLN B | 56 | 10.690 | 36.309 | 52.944 | 1.00 | 54.32 | N |

| | | | | | | | | | | | |
|---|---|---|---|---|---|---|---|---|---|---|---|
| ANISOU | 2093 | NE2 | GLN B | 56 | 6879 | 6860 | 6901 | 5 | 6 | 28 | N |

| | | | | | | | | | | |
|---|---|---|---|---|---|---|---|---|---|---|
| ATOM | 2094 | N | GLY B | 57 | 14.899 | 40.680 | 52.958 | 1.00 | 54.34 | N |

| | | | | | | | | | | | |
|---|---|---|---|---|---|---|---|---|---|---|---|
| ANISOU | 2094 | N | GLY B | 57 | 6876 | 6886 | 6885 | 69 | −26 | 51 | N |

| | | | | | | | | | | |
|---|---|---|---|---|---|---|---|---|---|---|
| ATOM | 2095 | CA | GLY B | 57 | 15.988 | 41.638 | 53.073 | 1.00 | 55.13 | C |

| | | | | | | | | | | | |
|---|---|---|---|---|---|---|---|---|---|---|---|
| ANISOU | 2095 | CA | GLY B | 57 | 6995 | 6945 | 7008 | −2 | −5 | 16 | C |

| | | | | | | | | | | |
|---|---|---|---|---|---|---|---|---|---|---|
| ATOM | 2096 | C | GLY B | 57 | 16.907 | 41.276 | 54.241 | 1.00 | 55.51 | C |

| | | | | | | | | | | | |
|---|---|---|---|---|---|---|---|---|---|---|---|
| ANISOU | 2096 | C | GLY B | 57 | 7060 | 7008 | 7025 | 14 | −22 | 39 | C |

| | | | | | | | | | | |
|---|---|---|---|---|---|---|---|---|---|---|
| ATOM | 2097 | O | GLY B | 57 | 17.584 | 42.131 | 54.812 | 1.00 | 56.13 | O |

| | | | | | | | | | | | |
|---|---|---|---|---|---|---|---|---|---|---|---|
| ANISOU | 2097 | O | GLY B | 57 | 7178 | 7006 | 7144 | −24 | −9 | 40 | O |

| | | | | | | | | | | |
|---|---|---|---|---|---|---|---|---|---|---|
| ATOM | 2098 | N | LYS B | 58 | 16.933 | 39.999 | 54.615 | 1.00 | 55.18 | N |

| | | | | | | | | | | | |
|---|---|---|---|---|---|---|---|---|---|---|---|
| ANISOU | 2098 | N | LYS B | 58 | 7016 | 6970 | 6980 | 27 | −38 | 3 | N |

| | | | | | | | | | | |
|---|---|---|---|---|---|---|---|---|---|---|
| ATOM | 2099 | CA | LYS B | 58 | 17.696 | 39.522 | 55.748 | 1.00 | 54.66 | C |

| | | | | | | | | | | | |
|---|---|---|---|---|---|---|---|---|---|---|---|
| ANISOU | 2099 | CA | LYS B | 58 | 6942 | 6883 | 6941 | 16 | −10 | −22 | C |

| | | | | | | | | | | |
|---|---|---|---|---|---|---|---|---|---|---|
| ATOM | 2100 | C | LYS B | 58 | 18.916 | 38.715 | 55.326 | 1.00 | 53.98 | C |

| | | | | | | | | | | | |
|---|---|---|---|---|---|---|---|---|---|---|---|
| ANISOU | 2100 | C | LYS B | 58 | 6864 | 6793 | 6851 | −26 | −23 | 41 | C |

| | | | | | | | | | | |
|---|---|---|---|---|---|---|---|---|---|---|
| ATOM | 2101 | O | LYS B | 58 | 18.947 | 38.065 | 54.287 | 1.00 | 53.68 | O |

| | | | | | | | | | | | |
|---|---|---|---|---|---|---|---|---|---|---|---|
| ANISOU | 2101 | O | LYS B | 58 | 6841 | 6721 | 6833 | −11 | −11 | 67 | O |

| | | | | | | | | | | |
|---|---|---|---|---|---|---|---|---|---|---|
| ATOM | 2102 | CB | LYS B | 58 | 16.824 | 38.670 | 56.683 | 1.00 | 55.17 | C |

| | | | | | | | | | | | |
|---|---|---|---|---|---|---|---|---|---|---|---|
| ANISOU | 2102 | CB | LYS B | 58 | 6977 | 7015 | 6972 | −15 | −29 | 45 | C |

| | | | | | | | | | | |
|---|---|---|---|---|---|---|---|---|---|---|
| ATOM | 2103 | CG | LYS B | 58 | 15.946 | 39.522 | 57.589 | 1.00 | 56.18 | C |

| | | | | | | | | | | | |
|---|---|---|---|---|---|---|---|---|---|---|---|
| ANISOU | 2103 | CG | LYS B | 58 | 7104 | 7131 | 7110 | 30 | 9 | −5 | C |

| | | | | | | | | | | |
|---|---|---|---|---|---|---|---|---|---|---|
| ATOM | 2104 | CD | LYS B | 58 | 14.899 | 38.691 | 58.321 | 1.00 | 56.75 | C |

| | | | | | | | | | | | |
|---|---|---|---|---|---|---|---|---|---|---|---|
| ANISOU | 2104 | CD | LYS B | 58 | 7176 | 7201 | 7188 | −20 | 18 | 14 | C |

| | | | | | | | | | | |
|---|---|---|---|---|---|---|---|---|---|---|
| ATOM | 2105 | CE | LYS B | 58 | 13.769 | 39.572 | 58.836 | 1.00 | 56.84 | C |

| | | | | | | | | | | | |
|---|---|---|---|---|---|---|---|---|---|---|---|
| ANISOU | 2105 | CE | LYS B | 58 | 7180 | 7227 | 7190 | −2 | −2 | 4 | C |

| | | | | | | | | | | |
|---|---|---|---|---|---|---|---|---|---|---|
| ATOM | 2106 | NZ | LYS B | 58 | 12.753 | 38.784 | 59.589 | 1.00 | 56.81 | N |

| | | | | | | | | | | | |
|---|---|---|---|---|---|---|---|---|---|---|---|
| ANISOU | 2106 | NZ | LYS B | 58 | 7186 | 7209 | 7191 | −18 | −19 | −2 | N |

| | | | | | | | | | | |
|---|---|---|---|---|---|---|---|---|---|---|
| ATOM | 2107 | N | GLU B | 59 | 19.912 | 38.785 | 56.202 | 1.00 | 53.55 | N |

| | | | | | | | | | | | |
|---|---|---|---|---|---|---|---|---|---|---|---|
| ANISOU | 2107 | N | GLU B | 59 | 6802 | 6702 | 6841 | 3 | 12 | 26 | N |

| | | | | | | | | | | |
|---|---|---|---|---|---|---|---|---|---|---|
| ATOM | 2108 | CA | GLU B | 59 | 21.151 | 38.052 | 55.964 | 1.00 | 53.20 | C |

| | | | | | | | | | | | |
|---|---|---|---|---|---|---|---|---|---|---|---|
| ANISOU | 2108 | CA | GLU B | 59 | 6759 | 6698 | 6756 | −25 | 1 | 31 | C |

| | | | | | | | | | | |
|---|---|---|---|---|---|---|---|---|---|---|
| ATOM | 2109 | C | GLU B | 59 | 20.927 | 36.584 | 56.321 | 1.00 | 52.61 | C |

| | | | | | | | | | | | |
|---|---|---|---|---|---|---|---|---|---|---|---|
| ANISOU | 2109 | C | GLU B | 59 | 6645 | 6654 | 6691 | −3 | −5 | −6 | C |

| | | | | | | | | | | |
|---|---|---|---|---|---|---|---|---|---|---|
| ATOM | 2110 | O | GLU B | 59 | 20.245 | 36.249 | 57.290 | 1.00 | 52.52 | O |

| | | | | | | | | | | | |
|---|---|---|---|---|---|---|---|---|---|---|---|
| ANISOU | 2110 | O | GLU B | 59 | 6593 | 6603 | 6758 | −72 | −14 | −7 | O |

| | | | | | | | | | | |
|---|---|---|---|---|---|---|---|---|---|---|
| ATOM | 2111 | CB | GLU B | 59 | 22.275 | 38.670 | 56.777 | 1.00 | 53.05 | C |

| | | | | | | | | | | | |
|---|---|---|---|---|---|---|---|---|---|---|---|
| ANISOU | 2111 | CB | GLU B | 59 | 6720 | 6688 | 6749 | 7 | 16 | 6 | C |

| | | | | | | | | | | |
|---|---|---|---|---|---|---|---|---|---|---|
| ATOM | 2112 | N | MET B | 60 | 21.545 | 35.709 | 55.554 | 1.00 | 51.89 | N |

| | | | | | | | | | | | |
|---|---|---|---|---|---|---|---|---|---|---|---|
| ANISOU | 2112 | N | MET B | 60 | 6548 | 6588 | 6579 | −25 | −23 | 35 | N |

| | | | | | | | | | | |
|---|---|---|---|---|---|---|---|---|---|---|
| ATOM | 2113 | CA | MET B | 60 | 21.509 | 34.279 | 55.744 | 1.00 | 51.70 | C |

| | | | | | | | | | | | |
|---|---|---|---|---|---|---|---|---|---|---|---|
| ANISOU | 2113 | CA | MET B | 60 | 6527 | 6576 | 6539 | −31 | 13 | −12 | C |

| | | | | | | | | | | |
|---|---|---|---|---|---|---|---|---|---|---|
| ATOM | 2114 | C | MET B | 60 | 22.019 | 33.785 | 57.084 | 1.00 | 52.12 | C |

| | | | | | | | | | | | |
|---|---|---|---|---|---|---|---|---|---|---|---|
| ANISOU | 2114 | C | MET B | 60 | 6605 | 6602 | 6595 | −25 | −14 | 22 | C |

| | | | | | | | | | | |
|---|---|---|---|---|---|---|---|---|---|---|
| ATOM | 2115 | O | MET B | 60 | 21.512 | 32.787 | 57.592 | 1.00 | 52.65 | O |

| | | | | | | | | | | | |
|---|---|---|---|---|---|---|---|---|---|---|---|
| ANISOU | 2115 | O | MET B | 60 | 6718 | 6658 | 6629 | −47 | 65 | 38 | O |

| | | | | | | | | | | |
|---|---|---|---|---|---|---|---|---|---|---|
| ATOM | 2116 | CB | MET B | 60 | 22.430 | 33.652 | 54.674 | 1.00 | 50.92 | C |

| | | | | | | | | | | | |
|---|---|---|---|---|---|---|---|---|---|---|---|
| ANISOU | 2116 | CB | MET B | 60 | 6441 | 6458 | 6448 | −53 | −52 | −12 | C |

| | | | | | | | | | | |
|---|---|---|---|---|---|---|---|---|---|---|
| ATOM | 2117 | CG | MET B | 60 | 21.681 | 33.554 | 53.340 | 1.00 | 49.61 | C |

| | | | | | | | | | | | |
|---|---|---|---|---|---|---|---|---|---|---|---|
| ANISOU | 2117 | CG | MET B | 60 | 6237 | 6262 | 6349 | −104 | 32 | 22 | C |

| | | | | | | | | | | |
|---|---|---|---|---|---|---|---|---|---|---|
| ATOM | 2118 | SD | MET B | 60 | 20.383 | 32.315 | 53.571 | 1.00 | 47.67 | S |

| | | | | | | | | | | | |
|---|---|---|---|---|---|---|---|---|---|---|---|
| ANISOU | 2118 | SD | MET B | 60 | 6200 | 6065 | 5846 | −53 | 88 | 25 | S |

| | | | | | | | | | | |
|---|---|---|---|---|---|---|---|---|---|---|
| ATOM | 2119 | CE | MET B | 60 | 21.295 | 30.826 | 53.148 | 1.00 | 47.57 | C |

| | | | | | | | | | | | |
|---|---|---|---|---|---|---|---|---|---|---|---|
| ANISOU | 2119 | CE | MET B | 60 | 5978 | 6078 | 6017 | −47 | −1 | 38 | C |

| | | | | | | | | | | |
|---|---|---|---|---|---|---|---|---|---|---|
| ATOM | 2120 | N | ASP B | 61 | 23.008 | 34.447 | 57.670 | 1.00 | 51.70 | N |

| | | | | | | | | | | | |
|---|---|---|---|---|---|---|---|---|---|---|---|
| ANISOU | 2120 | N | ASP B | 61 | 6571 | 6566 | 6506 | 3 | −16 | 42 | N |

| | | | | | | | | | | |
|---|---|---|---|---|---|---|---|---|---|---|
| ATOM | 2121 | CA | ASP B | 61 | 23.620 | 34.033 | 58.924 | 1.00 | 51.25 | C |

| | | | | | | | | | | | |
|---|---|---|---|---|---|---|---|---|---|---|---|
| ANISOU | 2121 | CA | ASP B | 61 | 6528 | 6495 | 6450 | 39 | 41 | −6 | C |

| | | | | | | | | | | |
|---|---|---|---|---|---|---|---|---|---|---|
| ATOM | 2122 | C | ASP B | 61 | 22.831 | 34.462 | 60.151 | 1.00 | 49.03 | C |

| | | | | | | | | | | | |
|---|---|---|---|---|---|---|---|---|---|---|---|
| ANISOU | 2122 | C | ASP B | 61 | 6135 | 6161 | 6334 | 4 | −53 | 87 | C |

| | | | | | | | | | | |
|---|---|---|---|---|---|---|---|---|---|---|
| ATOM | 2123 | O | ASP B | 61 | 23.231 | 34.204 | 61.290 | 1.00 | 49.30 | O |

| | | | | | | | | | | | |
|---|---|---|---|---|---|---|---|---|---|---|---|
| ANISOU | 2123 | O | ASP B | 61 | 6278 | 6190 | 6265 | −37 | −4 | −52 | O |

| | | | | | | | | | | |
|---|---|---|---|---|---|---|---|---|---|---|
| ATOM | 2124 | CB | ASP B | 61 | 25.069 | 34.545 | 58.999 | 1.00 | 53.17 | C |

| | | | | | | | | | | | |
|---|---|---|---|---|---|---|---|---|---|---|---|
| ANISOU | 2124 | CB | ASP B | 61 | 6602 | 6824 | 6775 | −24 | −17 | 31 | C |

| | | | | | | | | | | |
|---|---|---|---|---|---|---|---|---|---|---|
| ATOM | 2125 | CG | ASP B | 61 | 25.258 | 36.036 | 59.112 | 1.00 | 54.52 | C |

| | | | | | | | | | | | |
|---|---|---|---|---|---|---|---|---|---|---|---|
| ANISOU | 2125 | CG | ASP B | 61 | 6875 | 6880 | 6959 | −36 | −7 | 14 | C |

| | | | | | | | | | | |
|---|---|---|---|---|---|---|---|---|---|---|
| ATOM | 2126 | OD1 | ASP B | 61 | 24.273 | 36.810 | 59.043 | 1.00 | 55.35 | O |

| | | | | | | | | | | | |
|---|---|---|---|---|---|---|---|---|---|---|---|
| ANISOU | 2126 | OD1 | ASP B | 61 | 6932 | 6971 | 7126 | 7 | −25 | 47 | O |

| | | | | | | | | | | |
|---|---|---|---|---|---|---|---|---|---|---|
| ATOM | 2127 | OD2 | ASP B | 61 | 26.412 | 36.510 | 59.285 | 1.00 | 55.04 | O |

| | | | | | | | | | | | |
|---|---|---|---|---|---|---|---|---|---|---|---|
| ANISOU | 2127 | OD2 | ASP B | 61 | 6826 | 7093 | 6996 | −10 | 4 | 64 | O |

| | | | | | | | | | | |
|---|---|---|---|---|---|---|---|---|---|---|
| ATOM | 2128 | N | SER B | 62 | 21.732 | 35.163 | 59.923 | 1.00 | 46.10 | N |

| | | | | | | | | | | | |
|---|---|---|---|---|---|---|---|---|---|---|---|
| ANISOU | 2128 | N | SER B | 62 | 5834 | 5852 | 5830 | −146 | 113 | 15 | N |

| | | | | | | | | | | |
|---|---|---|---|---|---|---|---|---|---|---|
| ATOM | 2129 | CA | SER B | 62 | 20.838 | 35.649 | 60.948 | 1.00 | 43.53 | C |

| | | | | | | | | | | | |
|---|---|---|---|---|---|---|---|---|---|---|---|
| ANISOU | 2129 | CA | SER B | 62 | 5380 | 5499 | 5660 | −192 | −71 | 67 | C |

| | | | | | | | | | | |
|---|---|---|---|---|---|---|---|---|---|---|
| ATOM | 2130 | C | SER B | 62 | 19.565 | 34.787 | 60.955 | 1.00 | 39.77 | C |

| | | | | | | | | | | | |
|---|---|---|---|---|---|---|---|---|---|---|---|
| ANISOU | 2130 | C | SER B | 62 | 5122 | 4983 | 5004 | 38 | −49 | 50 | C |

| | | | | | | | | | | |
|---|---|---|---|---|---|---|---|---|---|---|
| ATOM | 2131 | O | SER B | 62 | 18.728 | 35.038 | 61.816 | 1.00 | 38.68 | O |

| | | | | | | | | | | | |
|---|---|---|---|---|---|---|---|---|---|---|---|
| ANISOU | 2131 | O | SER B | 62 | 4909 | 4953 | 4834 | −66 | −104 | 243 | O |

| | | | | | | | | | | |
|---|---|---|---|---|---|---|---|---|---|---|
| ATOM | 2132 | CB | SER B | 62 | 20.446 | 37.109 | 60.705 | 1.00 | 45.00 | C |

| | | | | | | | | | | | |
|---|---|---|---|---|---|---|---|---|---|---|---|
| ANISOU | 2132 | CB | SER B | 62 | 5683 | 5603 | 5811 | −42 | 3 | 59 | C |

| | | | | | | | | | | |
|---|---|---|---|---|---|---|---|---|---|---|
| ATOM | 2133 | OG | SER B | 62 | 19.630 | 37.220 | 59.536 | 1.00 | 47.01 | O |

| | | | | | | | | | | | |
|---|---|---|---|---|---|---|---|---|---|---|---|
| ANISOU | 2133 | OG | SER B | 62 | 6007 | 5955 | 5900 | −19 | −100 | 81 | O |

| | | | | | | | | | | |
|---|---|---|---|---|---|---|---|---|---|---|
| ATOM | 2134 | N | LEU B | 63 | 19.450 | 33.905 | 59.967 | 1.00 | 36.26 | N |

| | | | | | | | | | | | |
|---|---|---|---|---|---|---|---|---|---|---|---|
| ANISOU | 2134 | N | LEU B | 63 | 4488 | 4427 | 4862 | −86 | −40 | 304 | N |

| | | | | | | | | | | |
|---|---|---|---|---|---|---|---|---|---|---|
| ATOM | 2135 | CA | LEU B | 63 | 18.258 | 33.067 | 59.870 | 1.00 | 33.71 | C |

| | | | | | | | | | | | |
|---|---|---|---|---|---|---|---|---|---|---|---|
| ANISOU | 2135 | CA | LEU B | 83 | 4289 | 4044 | 4475 | 62 | 18 | 165 | C |

| | | | | | | | | | | |
|---|---|---|---|---|---|---|---|---|---|---|
| ATOM | 2136 | C | LEU B | 63 | 18.609 | 31.578 | 59.971 | 1.00 | 31.59 | C |

| | | | | | | | | | | | |
|---|---|---|---|---|---|---|---|---|---|---|---|
| ANISOU | 2136 | C | LEU B | 63 | 3950 | 3949 | 4104 | −55 | 90 | 280 | C |

| | | | | | | | | | | |
|---|---|---|---|---|---|---|---|---|---|---|
| ATOM | 2137 | O | LEU B | 63 | 19.568 | 31.099 | 59.371 | 1.00 | 31.78 | O |

| | | | | | | | | | | | |
|---|---|---|---|---|---|---|---|---|---|---|---|
| ANISOU | 2137 | O | LEU B | 63 | 4103 | 3811 | 4158 | 22 | 160 | 417 | O |

| | | | | | | | | | | |
|---|---|---|---|---|---|---|---|---|---|---|
| ATOM | 2138 | CB | LEU B | 63 | 17.522 | 33.173 | 58.529 | 1.00 | 34.61 | C |

| | | | | | | | | | | | |
|---|---|---|---|---|---|---|---|---|---|---|---|
| ANISOU | 2138 | CB | LEU B | 63 | 4385 | 4316 | 4448 | 26 | 33 | 98 | C |

| | | | | | | | | | | |
|---|---|---|---|---|---|---|---|---|---|---|
| ATOM | 2139 | CG | LEU B | 63 | 16.682 | 34.423 | 58.257 | 1.00 | 35.54 | C |

| | | | | | | | | | | | |
|---|---|---|---|---|---|---|---|---|---|---|---|
| ANISOU | 2139 | CG | LEU B | 63 | 4540 | 4433 | 4530 | 127 | −34 | 78 | C |

| | | | | | | | | | | |
|---|---|---|---|---|---|---|---|---|---|---|
| ATOM | 2140 | CD1 | LEU B | 63 | 16.227 | 34.429 | 56.803 | 1.00 | 35.42 | C |

| | | | | | | | | | | | |
|---|---|---|---|---|---|---|---|---|---|---|---|
| ANISOU | 2140 | CD1 | LEU B | 63 | 4525 | 4436 | 4498 | 70 | −11 | 53 | C |

| | | | | | | | | | | |
|---|---|---|---|---|---|---|---|---|---|---|
| ATOM | 2141 | CD2 | LEU B | 63 | 15.482 | 34.503 | 59.180 | 1.00 | 35.92 | C |

| | | | | | | | | | | | |
|---|---|---|---|---|---|---|---|---|---|---|---|
| ANISOU | 2141 | CD2 | LEU B | 63 | 4568 | 4534 | 4546 | 14 | −12 | 126 | C |

| | | | | | | | | | | |
|---|---|---|---|---|---|---|---|---|---|---|
| ATOM | 2142 | N | ARG B | 64 | 17.816 | 30.831 | 60.730 | 1.00 | 28.52 | N |

| | | | | | | | | | | | |
|---|---|---|---|---|---|---|---|---|---|---|---|
| ANISOU | 2142 | N | ARG B | 64 | 3433 | 3671 | 3733 | −28 | −93 | 74 | N |

| | | | | | | | | | | |
|---|---|---|---|---|---|---|---|---|---|---|
| ATOM | 2143 | CA | ARG B | 64 | 18.010 | 29.398 | 60.822 | 1.00 | 25.81 | C |

| | | | | | | | | | | | |
|---|---|---|---|---|---|---|---|---|---|---|---|
| ANISOU | 2143 | CA | ARG B | 64 | 3105 | 3587 | 3114 | 19 | −14 | 56 | C |

| | | | | | | | | | | |
|---|---|---|---|---|---|---|---|---|---|---|
| ATOM | 2144 | C | ARG B | 64 | 16.848 | 28.717 | 60.090 | 1.00 | 26.11 | C |

| | | | | | | | | | | | |
|---|---|---|---|---|---|---|---|---|---|---|---|
| ANISOU | 2144 | C | ARG B | 64 | 3216 | 3542 | 3163 | 33 | −1 | −39 | C |

| | | | | | | | | | | |
|---|---|---|---|---|---|---|---|---|---|---|
| ATOM | 2145 | O | ARG B | 64 | 15.688 | 29.012 | 60.406 | 1.00 | 27.40 | O |

| | | | | | | | | | | | |
|---|---|---|---|---|---|---|---|---|---|---|---|
| ANISOU | 2145 | O | ARG B | 64 | 3283 | 3730 | 3399 | 19 | −51 | −117 | O |

| | | | | | | | | | | |
|---|---|---|---|---|---|---|---|---|---|---|
| ATOM | 2146 | CB | ARG B | 64 | 17.989 | 28.920 | 62.295 | 1.00 | 26.53 | C |

| | | | | | | | | | | | |
|---|---|---|---|---|---|---|---|---|---|---|---|
| ANISOU | 2146 | CB | ARG B | 64 | 3394 | 3612 | 3073 | −34 | 23 | 60 | C |

| | | | | | | | | | | |
|---|---|---|---|---|---|---|---|---|---|---|
| ATOM | 2147 | CG | ARG B | 64 | 18.821 | 29.807 | 63.229 | 1.00 | 26.50 | C |

| | | | | | | | | | | | |
|---|---|---|---|---|---|---|---|---|---|---|---|
| ANISOU | 2147 | CG | ARG B | 64 | 3533 | 3550 | 2984 | −32 | 2 | 113 | C |

| | | | | | | | | | | |
|---|---|---|---|---|---|---|---|---|---|---|
| ATOM | 2148 | CD | ARG B | 64 | 20.293 | 29.729 | 62.903 | 1.00 | 27.50 | C |

| | | | | | | | | | | | |
|---|---|---|---|---|---|---|---|---|---|---|---|
| ANISOU | 2148 | CD | ARG B | 64 | 3535 | 3681 | 3234 | −126 | −63 | 134 | C |

| | | | | | | | | | | |
|---|---|---|---|---|---|---|---|---|---|---|
| ATOM | 2149 | NE | ARG B | 64 | 21.105 | 30.383 | 63.937 | 1.00 | 27.46 | N |

| | | | | | | | | | | | |
|---|---|---|---|---|---|---|---|---|---|---|---|
| ANISOU | 2149 | NE | ARG B | 64 | 3606 | 3654 | 3173 | −64 | −109 | 200 | N |

| | | | | | | | | | | |
|---|---|---|---|---|---|---|---|---|---|---|
| ATOM | 2150 | CZ | ARG B | 64 | 21.834 | 31.451 | 63.767 | 1.00 | 27.33 | C |

| | | | | | | | | | | | |
|---|---|---|---|---|---|---|---|---|---|---|---|
| ANISOU | 2150 | CZ | ARG B | 64 | 3492 | 3567 | 3325 | −93 | −116 | 60 | C |

| | | | | | | | | | | |
|---|---|---|---|---|---|---|---|---|---|---|
| ATOM | 2151 | NH1 | ARG B | 64 | 21.926 | 32.034 | 62.561 | 1.00 | 29.19 | N |

| | | | | | | | | | | | |
|---|---|---|---|---|---|---|---|---|---|---|---|
| ANISOU | 2151 | NH1 | ARG B | 64 | 3723 | 3917 | 3453 | −188 | 17 | 196 | N |

| | | | | | | | | | | |
|---|---|---|---|---|---|---|---|---|---|---|
| ATOM | 2152 | NH2 | ARG B | 64 | 22.509 | 31.932 | 64.798 | 1.00 | 26.04 | N |

| | | | | | | | | | | | |
|---|---|---|---|---|---|---|---|---|---|---|---|
| ANISOU | 2152 | NH2 | ARG B | 64 | 3462 | 3343 | 3088 | 73 | −45 | 219 | N |

| | | | | | | | | | | |
|---|---|---|---|---|---|---|---|---|---|---|
| ATOM | 2153 | N | PHE B | 65 | 17.150 | 27.845 | 59.156 | 1.00 | 24.96 | N |

| | | | | | | | | | | | |
|---|---|---|---|---|---|---|---|---|---|---|---|
| ANISOU | 2153 | N | PHE B | 65 | 3251 | 3243 | 2990 | 14 | −85 | 64 | N |

| | | | | | | | | | | |
|---|---|---|---|---|---|---|---|---|---|---|
| ATOM | 2154 | CA | PHE B | 65 | 16.116 | 27.166 | 58.385 | 1.00 | 25.66 | C |

| | | | | | | | | | | | |
|---|---|---|---|---|---|---|---|---|---|---|---|
| ANISOU | 2154 | CA | PHE B | 65 | 3183 | 3370 | 3197 | −48 | 1 | 93 | C |

| | | | | | | | | | | |
|---|---|---|---|---|---|---|---|---|---|---|
| ATOM | 2155 | C | PHE B | 65 | 15.971 | 25.753 | 58.935 | 1.00 | 25.10 | C |

| | | | | | | | | | | | |
|---|---|---|---|---|---|---|---|---|---|---|---|
| ANISOU | 2155 | C | PHE B | 65 | 3260 | 3368 | 2909 | −7 | −5 | 87 | C |

| | | | | | | | | | | |
|---|---|---|---|---|---|---|---|---|---|---|
| ATOM | 2156 | O | PHE B | 65 | 16.918 | 24.959 | 58.899 | 1.00 | 25.00 | O |

| | | | | | | | | | | | |
|---|---|---|---|---|---|---|---|---|---|---|---|
| ANISOU | 2156 | O | PHE B | 65 | 3165 | 3822 | 2513 | 70 | 148 | 188 | O |

| | | | | | | | | | | |
|---|---|---|---|---|---|---|---|---|---|---|
| ATOM | 2157 | CB | PHE B | 65 | 16.467 | 27.129 | 56.892 | 1.00 | 27.19 | C |

| | | | | | | | | | | | |
|---|---|---|---|---|---|---|---|---|---|---|---|
| ANISOU | 2157 | CB | PHE B | 65 | 3405 | 3704 | 3222 | 30 | 21 | −8 | C |

| | | | | | | | | | | |
|---|---|---|---|---|---|---|---|---|---|---|
| ATOM | 2158 | CG | PHE B | 65 | 16.501 | 28.473 | 56.236 | 1.00 | 29.68 | C |

| | | | | | | | | | | | |
|---|---|---|---|---|---|---|---|---|---|---|---|
| ANISOU | 2158 | CG | PHE B | 65 | 3870 | 3847 | 3558 | 2 | 8 | 92 | C |

| | | | | | | | | | | |
|---|---|---|---|---|---|---|---|---|---|---|
| ATOM | 2159 | CD1 | PHE B | 65 | 17.643 | 29.247 | 56.227 | 1.00 | 30.70 | C |

| | | | | | | | | | | | |
|---|---|---|---|---|---|---|---|---|---|---|---|
| ANISOU | 2159 | CD1 | PHE B | 65 | 3780 | 3994 | 3889 | −17 | −18 | 3 | C |

| | | | | | | | | | | |
|---|---|---|---|---|---|---|---|---|---|---|
| ATOM | 2160 | CD2 | PHE B | 65 | 15.366 | 28.933 | 55.565 | 1.00 | 30.32 | C |

| | | | | | | | | | | | |
|---|---|---|---|---|---|---|---|---|---|---|---|
| ANISOU | 2160 | CD2 | PHE B | 65 | 3722 | 3977 | 3823 | 50 | 50 | 55 | C |

| | | | | | | | | | | |
|---|---|---|---|---|---|---|---|---|---|---|
| ATOM | 2161 | CE1 | PHE B | 65 | 17.645 | 30.478 | 55.601 | 1.00 | 31.72 | C |

| | | | | | | | | | | | |
|---|---|---|---|---|---|---|---|---|---|---|---|
| ANISOU | 2161 | CE1 | PHE B | 65 | 3904 | 4119 | 4030 | −64 | 34 | 119 | C |

| | | | | | | | | | | |
|---|---|---|---|---|---|---|---|---|---|---|
| ATOM | 2162 | CE2 | PHE B | 65 | 15.369 | 30.171 | 54.941 | 1.00 | 31.21 | C |

| | | | | | | | | | | | |
|---|---|---|---|---|---|---|---|---|---|---|---|
| ANISOU | 2162 | CE2 | PHE B | 65 | 3981 | 4020 | 3859 | −30 | −13 | 63 | C |

| | | | | | | | | | | |
|---|---|---|---|---|---|---|---|---|---|---|
| ATOM | 2163 | CZ | PHE B | 65 | 16.518 | 30.945 | 54.947 | 1.00 | 31.82 | C |

| | | | | | | | | | | | |
|---|---|---|---|---|---|---|---|---|---|---|---|
| ANISOU | 2163 | CZ | PHE B | 65 | 4038 | 4097 | 3956 | −43 | −27 | 62 | C |

| | | | | | | | | | | |
|---|---|---|---|---|---|---|---|---|---|---|
| ATOM | 2164 | N | LEU B | 66 | 14.805 | 25.470 | 59.517 | 1.00 | 24.98 | N |

| | | | | | | | | | | | |
|---|---|---|---|---|---|---|---|---|---|---|---|
| ANISOU | 2164 | N | LEU B | 66 | 3149 | 3442 | 2899 | 96 | −32 | 17 | N |

| | | | | | | | | | | |
|---|---|---|---|---|---|---|---|---|---|---|
| ATOM | 2165 | CA | LEU B | 66 | 14.597 | 24.165 | 60.144 | 1.00 | 25.89 | C |

| | | | | | | | | | | | |
|---|---|---|---|---|---|---|---|---|---|---|---|
| ANISOU | 2165 | CA | LEU B | 66 | 3215 | 3385 | 3239 | −43 | −104 | −26 | C |

| | | | | | | | | | | |
|---|---|---|---|---|---|---|---|---|---|---|
| ATOM | 2166 | C | LEU B | 66 | 13.529 | 23.314 | 59.446 | 1.00 | 26.86 | C |

| | | | | | | | | | | | |
|---|---|---|---|---|---|---|---|---|---|---|---|
| ANSOU | 2166 | C | LEU B | 66 | 3401 | 3441 | 3366 | 11 | −94 | −46 | C |

| | | | | | | | | | | |
|---|---|---|---|---|---|---|---|---|---|---|
| ATOM | 2167 | O | LEU B | 66 | 12.525 | 23.841 | 58.991 | 1.00 | 25.08 | O |

| | | | | | | | | | | | |
|---|---|---|---|---|---|---|---|---|---|---|---|
| ANISOU | 2167 | O | LEU B | 66 | 3185 | 3287 | 3056 | 168 | 90 | 32 | O |

| | | | | | | | | | | |
|---|---|---|---|---|---|---|---|---|---|---|
| ATOM | 2168 | CB | LEU B | 66 | 14.094 | 24.367 | 61.583 | 1.00 | 27.45 | C |

| | | | | | | | | | | | |
|---|---|---|---|---|---|---|---|---|---|---|---|
| ANISOU | 2168 | CB | LEU B | 66 | 3450 | 3620 | 3359 | −57 | 33 | 12 | C |

| | | | | | | | | | | |
|---|---|---|---|---|---|---|---|---|---|---|
| ATOM | 2169 | CG | LEU B | 66 | 14.853 | 25.302 | 62.513 | 1.00 | 28.71 | C |

| | | | | | | | | | | | |
|---|---|---|---|---|---|---|---|---|---|---|---|
| ANISOU | 2169 | CG | LUU B | 66 | 3449 | 3948 | 3512 | −73 | −71 | −65 | C |

| | | | | | | | | | | |
|---|---|---|---|---|---|---|---|---|---|---|
| ATOM | 2170 | CD1 | LEU B | 66 | 14.350 | 25.151 | 63.959 | 1.00 | 29.40 | C |

| | | | | | | | | | | | |
|---|---|---|---|---|---|---|---|---|---|---|---|
| ANISOU | 2170 | CD1 | LEU B | 66 | 3581 | 3947 | 3641 | −168 | 134 | −118 | C |

| | | | | | | | | | | |
|---|---|---|---|---|---|---|---|---|---|---|
| ATOM | 2171 | CD2 | LEU B | 66 | 16.353 | 25.078 | 62.517 | 1.00 | 28.52 | C |

| | | | | | | | | | | | |
|---|---|---|---|---|---|---|---|---|---|---|---|
| ANISOU | 2171 | CD2 | LEU B | 66 | 3518 | 3884 | 3435 | 90 | −10 | −5 | C |

| | | | | | | | | | | |
|---|---|---|---|---|---|---|---|---|---|---|
| ATOM | 2172 | N | TYR B | 67 | 13.776 | 22.012 | 59.487 | 1.00 | 26.50 | N |

| | | | | | | | | | | | |
|---|---|---|---|---|---|---|---|---|---|---|---|
| ANISOU | 2172 | N | TYR B | 67 | 3492 | 3391 | 3184 | −11 | 32 | −14 | N |

| | | | | | | | | | | |
|---|---|---|---|---|---|---|---|---|---|---|
| ATOM | 2173 | CA | TYR B | 67 | 12.813 | 21.076 | 58.923 | 1.00 | 27.08 | C |

| | | | | | | | | | | | |
|---|---|---|---|---|---|---|---|---|---|---|---|
| ANISOU | 2173 | CA | TYR B | 67 | 3458 | 3523 | 3306 | −66 | 6 | −29 | C |

| | | | | | | | | | | |
|---|---|---|---|---|---|---|---|---|---|---|
| ATOM | 2174 | C | TYR B | 67 | 12.511 | 20.057 | 60.012 | 1.00 | 25.68 | C |

| | | | | | | | | | | | |
|---|---|---|---|---|---|---|---|---|---|---|---|
| ANISOU | 2174 | C | TYR B | 67 | 3271 | 3392 | 3096 | −78 | −94 | −146 | C |

| | | | | | | | | | | |
|---|---|---|---|---|---|---|---|---|---|---|
| ATOM | 2175 | O | TYR B | 67 | 13.345 | 19.256 | 60.402 | 1.00 | 24.58 | O |

| | | | | | | | | | | | |
|---|---|---|---|---|---|---|---|---|---|---|---|
| ANISOU | 2175 | O | TYR B | 67 | 3156 | 3434 | 2748 | −63 | 55 | −352 | O |

| | | | | | | | | | | |
|---|---|---|---|---|---|---|---|---|---|---|
| ATOM | 2176 | CB | TYR B | 67 | 13.360 | 20.413 | 57.648 | 1.00 | 28.18 | C |

| | | | | | | | | | | | |
|---|---|---|---|---|---|---|---|---|---|---|---|
| ANISOU | 2176 | CB | TYR B | 67 | 3622 | 3669 | 3416 | 30 | 83 | −76 | C |

| | | | | | | | | | | |
|---|---|---|---|---|---|---|---|---|---|---|
| ATOM | 2177 | CG | TYR B | 67 | 12.490 | 19.221 | 57.259 | 1.00 | 28.18 | C |

| | | | | | | | | | | | |
|---|---|---|---|---|---|---|---|---|---|---|---|
| ANISOU | 2177 | CG | TYR B | 67 | 3741 | 3612 | 3353 | 12 | 110 | −29 | C |

| | | | | | | | | | | |
|---|---|---|---|---|---|---|---|---|---|---|
| ATOM | 2178 | CD1 | TYR B | 67 | 11.175 | 19.433 | 56.866 | 1.00 | 28.95 | C |

| | | | | | | | | | | | |
|---|---|---|---|---|---|---|---|---|---|---|---|
| ANISOU | 2178 | CD1 | TYR B | 67 | 3756 | 3712 | 3532 | −84 | 35 | −42 | C |

| | | | | | | | | | | |
|---|---|---|---|---|---|---|---|---|---|---|
| ATOM | 2179 | CD2 | TYR B | 67 | 12.989 | 17.925 | 57.312 | 1.00 | 27.79 | C |

| | | | | | | | | | | | |
|---|---|---|---|---|---|---|---|---|---|---|---|
| ANISOU | 2179 | CD2 | TYR B | 67 | 3770 | 3595 | 3195 | 3 | 78 | 49 | C |

| | | | | | | | | | | |
|---|---|---|---|---|---|---|---|---|---|---|
| ATOM | 2180 | CE1 | TYR B | 67 | 10.352 | 18.367 | 56.521 | 1.00 | 29.96 | C |

| | | | | | | | | | | | |
|---|---|---|---|---|---|---|---|---|---|---|---|
| ANISOU | 2180 | CE1 | TYR B | 67 | 4031 | 3710 | 3643 | −82 | −40 | −93 | C |

| | | | | | | | | | | |
|---|---|---|---|---|---|---|---|---|---|---|
| ATOM | 2181 | CE2 | TYR B | 67 | 12.179 | 16.855 | 56.979 | 1.00 | 29.12 | C |

| | | | | | | | | | | | |
|---|---|---|---|---|---|---|---|---|---|---|---|
| ANISOU | 2181 | CE2 | TYR B | 67 | 3882 | 3755 | 3428 | −85 | 41 | 11 | C |

| | | | | | | | | | | |
|---|---|---|---|---|---|---|---|---|---|---|
| ATOM | 2182 | CZ | TYR B | 67 | 10.879 | 17.091 | 56.599 | 1.00 | 29.69 | C |

| | | | | | | | | | | | |
|---|---|---|---|---|---|---|---|---|---|---|---|
| ANISOU | 2182 | CZ | TYR B | 67 | 3880 | 3765 | 3635 | −19 | −29 | 15 | C |

| | | | | | | | | | | |
|---|---|---|---|---|---|---|---|---|---|---|
| ATOM | 2183 | OH | TYR B | 67 | 10.053 | 16.050 | 56.259 | 1.00 | 32.14 | O |

| | | | | | | | | | | | |
|---|---|---|---|---|---|---|---|---|---|---|---|
| ANISOU | 2183 | OH | TYR B | 67 | 4228 | 3907 | 4076 | −207 | −125 | −40 | O |

| | | | | | | | | | | |
|---|---|---|---|---|---|---|---|---|---|---|
| ATOM | 2184 | N | ASP B | 68 | 11.328 | 20.184 | 60.624 | 1.00 | 25.56 | N |

| | | | | | | | | | | | |
|---|---|---|---|---|---|---|---|---|---|---|---|
| ANISOU | 2184 | N | ASP B | 68 | 3323 | 3377 | 3013 | −64 | −85 | −220 | N |

| | | | | | | | | | | |
|---|---|---|---|---|---|---|---|---|---|---|
| ATOM | 2185 | CA | ASP B | 68 | 10.923 | 19.276 | 61.701 | 1.00 | 27.04 | C |

| | | | | | | | | | | | |
|---|---|---|---|---|---|---|---|---|---|---|---|
| ANISOU | 2185 | CA | ASP B | 68 | 3416 | 3539 | 3318 | −89 | −189 | −6 | C |

| | | | | | | | | | | |
|---|---|---|---|---|---|---|---|---|---|---|
| ATOM | 2186 | C | ASP B | 68 | 11.935 | 19.318 | 62.854 | 1.00 | 25.55 | C |

| | | | | | | | | | | | |
|---|---|---|---|---|---|---|---|---|---|---|---|
| ANISOU | 2186 | C | ASP B | 68 | 3276 | 3275 | 3155 | −13 | −99 | 12 | C |

| | | | | | | | | | | |
|---|---|---|---|---|---|---|---|---|---|---|
| ATOM | 2187 | O | ASP B | 68 | 12.308 | 18.287 | 63.430 | 1.00 | 24.29 | O |

| | | | | | | | | | | | |
|---|---|---|---|---|---|---|---|---|---|---|---|
| ANISOU | 2187 | O | ASP B | 68 | 2889 | 3283 | 3057 | −89 | −100 | 81 | O |

| | | | | | | | | | | |
|---|---|---|---|---|---|---|---|---|---|---|
| ATOM | 2188 | CB | ASP B | 68 | 10.787 | 17.848 | 61.173 | 1.00 | 29.17 | C |

| | | | | | | | | | | | |
|---|---|---|---|---|---|---|---|---|---|---|---|
| ANISOU | 2188 | CB | ASP B | 68 | 3729 | 3609 | 3746 | 2 | −118 | −136 | C |

| | | | | | | | | | | |
|---|---|---|---|---|---|---|---|---|---|---|
| ATOM | 2189 | CG | ASP B | 68 | 9.492 | 17.622 | 60.395 | 1.00 | 31.97 | C |

| | | | | | | | | | | | |
|---|---|---|---|---|---|---|---|---|---|---|---|
| ANISOU | 2189 | CG | ASP B | 68 | 3862 | 4198 | 4086 | −31 | −185 | −96 | C |

| | | | | | | | | | | |
|---|---|---|---|---|---|---|---|---|---|---|
| ATOM | 2190 | OD1 | ASP B | 68 | 8.669 | 18.547 | 60.309 | 1.00 | 31.80 | O |

| | | | | | | | | | | | |
|---|---|---|---|---|---|---|---|---|---|---|---|
| ANISOU | 2190 | OD1 | ASP B | 68 | 4010 | 4054 | 4019 | −47 | −294 | −70 | O |

| | | | | | | | | | | |
|---|---|---|---|---|---|---|---|---|---|---|
| ATOM | 2191 | OD2 | ASP B | 68 | 9.343 | 16.483 | 59.886 | 1.00 | 33.94 | O |

| | | | | | | | | | | | |
|---|---|---|---|---|---|---|---|---|---|---|---|
| ANISOU | 2191 | OD2 | ASP B | 68 | 4243 | 4375 | 4279 | −212 | −207 | −225 | O |

| | | | | | | | | | | |
|---|---|---|---|---|---|---|---|---|---|---|
| ATOM | 2192 | N | GLY B | 69 | 12.460 | 20.510 | 63.090 | 1.00 | 24.44 | N |

| | | | | | | | | | | |
|---|---|---|---|---|---|---|---|---|---|---|
| ANISOU | 2192 | N | GLY B | 69 | 3073 | 3243 | 2968 | −80 | −149 | 62 N |

| | | | | | | | | | | |
|---|---|---|---|---|---|---|---|---|---|---|
| ATOM | 2193 | CA | GLY B | 69 | 13.428 | 20.760 | 64.143 | 1.00 | 23.71 | C |

| | | | | | | | | | | | |
|---|---|---|---|---|---|---|---|---|---|---|---|
| ANISOU | 2193 | CA | GLY B | 69 | 3004 | 3167 | 2839 | 112 | −78 | −53 | C |

| | | | | | | | | | | |
|---|---|---|---|---|---|---|---|---|---|---|
| ATOM | 2194 | C | GLY B | 69 | 14.873 | 20.590 | 63.761 | 1.00 | 23.88 | C |

| | | | | | | | | | | | |
|---|---|---|---|---|---|---|---|---|---|---|---|
| ANISOU | 2194 | C | GLY B | 69 | 2884 | 3229 | 2962 | −103 | −124 | −147 | C |

| | | | | | | | | | | |
|---|---|---|---|---|---|---|---|---|---|---|
| ATOM | 2195 | O | GLY B | 69 | 15.755 | 20.896 | 64.594 | 1.00 | 24.06 | O |

| | | | | | | | | | | | |
|---|---|---|---|---|---|---|---|---|---|---|---|
| ANISOU | 2195 | O | GLY B | 69 | 2885 | 3344 | 2914 | 186 | −167 | −406 | O |

| | | | | | | | | | | |
|---|---|---|---|---|---|---|---|---|---|---|
| ATOM | 2196 | N | ILE B | 70 | 15.170 | 20.055 | 62.592 | 1.00 | 23.99 | N |

| | | | | | | | | | | | |
|---|---|---|---|---|---|---|---|---|---|---|---|
| ANISOU | 2196 | N | ILE B | 70 | 2900 | 3280 | 2936 | −39 | −44 | −185 | N |

| | | | | | | | | | | |
|---|---|---|---|---|---|---|---|---|---|---|
| ATOM | 2197 | CA | ILE B | 70 | 16.563 | 19.855 | 62.146 | 1.00 | 25.40 | C |

| | | | | | | | | | | | |
|---|---|---|---|---|---|---|---|---|---|---|---|
| ANISOU | 2197 | CA | ILE B | 70 | 3104 | 3353 | 3194 | −34 | 8 | −114 | C |

| | | | | | | | | | | |
|---|---|---|---|---|---|---|---|---|---|---|
| ATOM | 2198 | C | ILE B | 70 | 17.077 | 21.096 | 61.443 | 1.00 | 25.08 | C |

| | | | | | | | | | | | |
|---|---|---|---|---|---|---|---|---|---|---|---|
| ANISOU | 2198 | C | ILE B | 70 | 2947 | 3416 | 3167 | −13 | −60 | 2 | C |

| | | | | | | | | | | |
|---|---|---|---|---|---|---|---|---|---|---|
| ATOM | 2199 | O | ILE B | 70 | 16.453 | 21.601 | 60.493 | 1.00 | 24.99 | O |

| | | | | | | | | | | | |
|---|---|---|---|---|---|---|---|---|---|---|---|
| ANISOU | 2199 | O | ILE B | 70 | 2896 | 3652 | 2949 | −181 | −243 | −174 | O |

| | | | | | | | | | | |
|---|---|---|---|---|---|---|---|---|---|---|
| ATOM | 2200 | CB | ILE B | 70 | 16.623 | 18.674 | 61.143 | 1.00 | 26.14 | C |

| | | | | | | | | | | | |
|---|---|---|---|---|---|---|---|---|---|---|---|
| ANISOU | 2200 | CB | ILE B | 70 | 3251 | 3398 | 3282 | 58 | 108 | −158 | C |

| | | | | | | | | | | |
|---|---|---|---|---|---|---|---|---|---|---|
| ATOM | 2201 | CG1 | ILE B | 70 | 16.156 | 17.377 | 61.801 | 1.00 | 27.39 | C |

| | | | | | | | | | | | |
|---|---|---|---|---|---|---|---|---|---|---|---|
| ANISOU | 2201 | CG1 | ILE B | 70 | 3550 | 3439 | 3417 | 39 | 14 | −133 | C |

| | | | | | | | | | | |
|---|---|---|---|---|---|---|---|---|---|---|
| ATOM | 2202 | CG2 | ILE B | 70 | 18.054 | 18.550 | 60.587 | 1.00 | 25.18 | C |

| | | | | | | | | | | | |
|---|---|---|---|---|---|---|---|---|---|---|---|
| ANISOU | 2202 | CG2 | ILE B | 70 | 3176 | 3286 | 3105 | 180 | −12 | −222 | C |

| | | | | | | | | | | |
|---|---|---|---|---|---|---|---|---|---|---|
| ATOM | 2203 | CD1 | ILE B | 70 | 15.876 | 16.352 | 60.695 | 1.00 | 28.46 | C |

| | | | | | | | | | | | |
|---|---|---|---|---|---|---|---|---|---|---|---|
| ANISOU | 2203 | CD1 | ILE B | 70 | 3730 | 3619 | 3462 | 7 | 7 | −215 | C |

| | | | | | | | | | | |
|---|---|---|---|---|---|---|---|---|---|---|
| ATOM | 2204 | N | ARG B | 71 | 18.229 | 21.606 | 61.881 | 1.00 | 23.27 | N |

| | | | | | | | | | | | |
|---|---|---|---|---|---|---|---|---|---|---|---|
| ANISOU | 2204 | N | ARG B | 71 | 2979 | 2921 | 2943 | 12 | −39 | −168 | N |

| | | | | | | | | | | |
|---|---|---|---|---|---|---|---|---|---|---|
| ATOM | 2205 | CA | ARG B | 71 | 18.819 | 22.806 | 61.317 | 1.00 | 25.13 | C |

| | | | | | | | | | | | |
|---|---|---|---|---|---|---|---|---|---|---|---|
| ANISOU | 2205 | CA | ARG B | 71 | 3253 | 3205 | 3091 | −57 | −37 | −42 | C |

| | | | | | | | | | | |
|---|---|---|---|---|---|---|---|---|---|---|
| ATOM | 2206 | C | ARG B | 71 | 19.495 | 22.455 | 59.998 | 1.00 | 26.72 | C |

| | | | | | | | | | | | |
|---|---|---|---|---|---|---|---|---|---|---|---|
| ANISOU | 2206 | C | ARG B | 71 | 3226 | 3613 | 3314 | −28 | 150 | 8 | C |

| | | | | | | | | | | |
|---|---|---|---|---|---|---|---|---|---|---|
| ATOM | 2207 | O | ARG B | 71 | 20.522 | 21.782 | 59.974 | 1.00 | 27.96 | O |

| | | | | | | | | | | | |
|---|---|---|---|---|---|---|---|---|---|---|---|
| ANISOU | 2207 | O | ARG B | 71 | 3497 | 3836 | 3291 | 231 | −78 | −32 | O |

| | | | | | | | | | | |
|---|---|---|---|---|---|---|---|---|---|---|
| ATOM | 2208 | CB | ARG B | 71 | 19.868 | 23.329 | 62.302 | 1.00 | 24.76 | C |

| | | | | | | | | | | | |
|---|---|---|---|---|---|---|---|---|---|---|---|
| ANISOU | 2208 | CB | ARG B | 71 | 3195 | 3105 | 3109 | −10 | −99 | 2 | C |

| | | | | | | | | | | |
|---|---|---|---|---|---|---|---|---|---|---|
| ATOM | 2209 | CG | ARG B | 71 | 20.440 | 24.691 | 61.899 | 1.00 | 25.29 | C |

| | | | | | | | | | | | |
|---|---|---|---|---|---|---|---|---|---|---|---|
| ANISOU | 2209 | CG | ARG B | 71 | 3258 | 3194 | 3159 | −82 | 44 | −72 | C |

| | | | | | | | | | | |
|---|---|---|---|---|---|---|---|---|---|---|
| ATOM | 2210 | CD | ARG B | 71 | 21.710 | 24.989 | 62.720 | 1.00 | 26.18 | C |

| | | | | | | | | | | | |
|---|---|---|---|---|---|---|---|---|---|---|---|
| ANISOU | 2210 | CD | ARG B | 71 | 3244 | 3466 | 3238 | −64 | −10 | −74 | C |

| | | | | | | | | | | |
|---|---|---|---|---|---|---|---|---|---|---|
| ATOM | 2211 | NE | ARG B | 71 | 21.245 | 25.193 | 64.120 | 1.00 | 26.72 | N |

| | | | | | | | | | | | |
|---|---|---|---|---|---|---|---|---|---|---|---|
| ANISOU | 2211 | NE | ARG B | 71 | 3435 | 3475 | 3244 | 80 | −3 | −111 | N |

| | | | | | | | | | | |
|---|---|---|---|---|---|---|---|---|---|---|
| ATOM | 2212 | CZ | ARG B | 71 | 21.488 | 26.324 | 64.774 | 1.00 | 26.12 | C |

| | | | | | | | | | | | |
|---|---|---|---|---|---|---|---|---|---|---|---|
| ANISOU | 2212 | CZ | ARG B | 71 | 3232 | 3521 | 3173 | 32 | −2 | −72 | C |

| | | | | | | | | | | |
|---|---|---|---|---|---|---|---|---|---|---|
| ATOM | 2213 | NH1 | ARG B | 71 | 22.159 | 27.310 | 64.198 | 1.00 | 27.62 | N |

| | | | | | | | | | | | |
|---|---|---|---|---|---|---|---|---|---|---|---|
| ANISOU | 2213 | NH1 | ARG B | 71 | 3751 | 3409 | 3336 | −10 | −90 | 20 | N |

| | | | | | | | | | | |
|---|---|---|---|---|---|---|---|---|---|---|
| ATOM | 2214 | NH2 | ARG B | 71 | 21.009 | 26.447 | 66.019 | 1.00 | 24.03 | N |

| | | | | | | | | | | | |
|---|---|---|---|---|---|---|---|---|---|---|---|
| ANISOU | 2214 | NH2 | ARG B | 71 | 3088 | 3164 | 2879 | 212 | −289 | −99 | N |

| | | | | | | | | | | |
|---|---|---|---|---|---|---|---|---|---|---|
| ATOM | 2215 | N | ILE B | 72 | 18.888 | 22.889 | 58.917 | 1.00 | 27.53 | N |

| | | | | | | | | | | | |
|---|---|---|---|---|---|---|---|---|---|---|---|
| ANISOU | 2215 | N | ILE B | 72 | 3315 | 3727 | 3418 | 6 | 71 | 57 | N |

| | | | | | | | | | | |
|---|---|---|---|---|---|---|---|---|---|---|
| ATOM | 2216 | CA | ILE B | 72 | 19.378 | 22.637 | 57.571 | 1.00 | 28.63 | C |

| | | | | | | | | | | | |
|---|---|---|---|---|---|---|---|---|---|---|---|
| ANISOU | 2216 | CA | ILE B | 72 | 3465 | 3869 | 3542 | 84 | 92 | −7 | C |

| | | | | | | | | | | |
|---|---|---|---|---|---|---|---|---|---|---|
| ATOM | 2217 | C | ILE B | 72 | 20.679 | 23.392 | 57.304 | 1.00 | 30.30 | C |

| | | | | | | | | | | | |
|---|---|---|---|---|---|---|---|---|---|---|---|
| ANISOU | 2217 | C | ILE B | 72 | 3693 | 3999 | 3819 | −34 | 73 | 45 | C |

| | | | | | | | | | | |
|---|---|---|---|---|---|---|---|---|---|---|
| ATOM | 2218 | O | ILE B | 72 | 20.893 | 24.548 | 57.640 | 1.00 | 29.09 | O |

| | | | | | | | | | | | |
|---|---|---|---|---|---|---|---|---|---|---|---|
| ANISOU | 2218 | O | ILE B | 72 | 3254 | 3987 | 3811 | 24 | 185 | 70 | O |

| | | | | | | | | | | |
|---|---|---|---|---|---|---|---|---|---|---|
| ATOM | 2219 | CB | ILE B | 72 | 18.333 | 23.078 | 56.537 | 1.00 | 29.16 | C |

| | | | | | | | | | | | |
|---|---|---|---|---|---|---|---|---|---|---|---|
| ANISOU | 2219 | CB | ILE B | 72 | 3555 | 3934 | 3592 | −32 | 10 | 44 | C |

| | | | | | | | | | | |
|---|---|---|---|---|---|---|---|---|---|---|
| ATOM | 2220 | CG1 | ILE B | 72 | 17.010 | 22.329 | 56.770 | 1.00 | 29.08 | C |

| | | | | | | | | | | | |
|---|---|---|---|---|---|---|---|---|---|---|---|
| ANISOU | 2220 | CG1 | ILE B | 72 | 3377 | 3904 | 3489 | 20 | −65 | 29 | C |

| | | | | | | | | | | |
|---|---|---|---|---|---|---|---|---|---|---|
| ATOM | 2221 | CG2 | ILE B | 72 | 18.784 | 22.783 | 55.120 | 1.00 | 29.87 | C |

| | | | | | | | | | | | |
|---|---|---|---|---|---|---|---|---|---|---|---|
| ANISOU | 2221 | CG2 | ILE B | 72 | 3734 | 4025 | 3590 | 121 | −2 | 6 | C |

| | | | | | | | | | | |
|---|---|---|---|---|---|---|---|---|---|---|
| ATOM | 2222 | CD1 | ILE B | 72 | 15.864 | 23.035 | 56.009 | 1.00 | 27.23 | C |

| | | | | | | | | | | | |
|---|---|---|---|---|---|---|---|---|---|---|---|
| ANISOU | 2222 | CD1 | ILE B | 72 | 3428 | 3606 | 3314 | 22 | −74 | 35 | C |

| | | | | | | | | | | |
|---|---|---|---|---|---|---|---|---|---|---|
| ATOM | 2223 | N | GLN B | 73 | 21.553 | 22.631 | 56.658 | 1.00 | 32.41 | N |

| | | | | | | | | | | | |
|---|---|---|---|---|---|---|---|---|---|---|---|
| ANISOU | 2223 | N | GLN B | 73 | 3967 | 4343 | 4005 | 225 | 82 | 46 | N |

| | | | | | | | | | | |
|---|---|---|---|---|---|---|---|---|---|---|
| ATOM | 2224 | CA | GLN B | 73 | 22.830 | 23.207 | 56.198 | 1.00 | 35.61 | C |

| | | | | | | | | | | | |
|---|---|---|---|---|---|---|---|---|---|---|---|
| ANISOU | 2224 | CA | GLN B | 73 | 4338 | 4637 | 4556 | −118 | 143 | 98 | C |

| | | | | | | | | | | |
|---|---|---|---|---|---|---|---|---|---|---|
| ATOM | 2225 | C | GLN B | 73 | 22.603 | 23.430 | 54.694 | 1.00 | 37.25 | C |

| | | | | | | | | | | | |
|---|---|---|---|---|---|---|---|---|---|---|---|
| ANISOU | 2225 | C | GLN B | 73 | 4631 | 4894 | 4628 | 0 | 2 | 29 | C |

| | | | | | | | | | | |
|---|---|---|---|---|---|---|---|---|---|---|
| ATOM | 2226 | O | GLN B | 73 | 22.109 | 22.545 | 53.991 | 1.00 | 36.37 | O |

| | | | | | | | | | | | |
|---|---|---|---|---|---|---|---|---|---|---|---|
| ANISOU | 2226 | O | GLN B | 73 | 4495 | 4824 | 4500 | −11 | 144 | 56 | O |

| | | | | | | | | | | |
|---|---|---|---|---|---|---|---|---|---|---|
| ATOM | 2227 | CB | GLN B | 73 | 23.977 | 22.265 | 56.532 | 1.00 | 37.02 | C |

| | | | | | | | | | | | |
|---|---|---|---|---|---|---|---|---|---|---|---|
| ANISOU | 2227 | CB | GLN B | 73 | 4602 | 4820 | 4645 | 71 | 59 | 36 | C |

| | | | | | | | | | | |
|---|---|---|---|---|---|---|---|---|---|---|
| ATOM | 2228 | CG | GLN B | 73 | 25.375 | 22.878 | 56.447 | 1.00 | 38.88 | C |

| | | | | | | | | | | | |
|---|---|---|---|---|---|---|---|---|---|---|---|
| ANISOU | 2228 | CG | GLN B | 73 | 4790 | 5080 | 4901 | −61 | 33 | −20 | C |

| | | | | | | | | | | |
|---|---|---|---|---|---|---|---|---|---|---|
| ATOM | 2229 | CD | GLN B | 73 | 25.906 | 22.962 | 55.028 | 1.00 | 39.95 | C |

| | | | | | | | | | | | |
|---|---|---|---|---|---|---|---|---|---|---|---|
| ANISOU | 2229 | CD | GLN B | 73 | 5035 | 5183 | 4960 | −9 | 63 | 6 | C |

| | | | | | | | | | | |
|---|---|---|---|---|---|---|---|---|---|---|
| ATOM | 2230 | OE1 | GLN B | 73 | 25.656 | 22.086 | 54.197 | 1.00 | 40.10 | O |

| | | | | | | | | | | | |
|---|---|---|---|---|---|---|---|---|---|---|---|
| ANISOU | 2230 | OE1 | GLN B | 73 | 5044 | 5390 | 4803 | −70 | 19 | −15 | O |

| | | | | | | | | | | |
|---|---|---|---|---|---|---|---|---|---|---|
| ATOM | 2231 | NE2 | GLN B | 73 | 26.645 | 24.024 | 54.736 | 1.00 | 40.37 | N |

| | | | | | | | | | | | |
|---|---|---|---|---|---|---|---|---|---|---|---|
| ANISOU | 2231 | NE2 | GLN B | 73 | 5030 | 5299 | 5011 | −66 | 62 | 43 | N |

| | | | | | | | | | | |
|---|---|---|---|---|---|---|---|---|---|---|
| ATOM | 2232 | N | ALA B | 74 | 22.920 | 24.646 | 54.235 | 1.00 | 39.00 | N |

| | | | | | | | | | | | |
|---|---|---|---|---|---|---|---|---|---|---|---|
| ANISOU | 2232 | N | ALA B | 74 | 4856 | 4933 | 5027 | −14 | 76 | 81 | N |

| | | | | | | | | | | |
|---|---|---|---|---|---|---|---|---|---|---|
| ATOM | 2233 | CA | ALA B | 74 | 22.697 | 24.979 | 52.817 | 1.00 | 41.23 | C |

| | | | | | | | | | | | |
|---|---|---|---|---|---|---|---|---|---|---|---|
| ANISOU | 2233 | CA | ALA B | 74 | 5221 | 5314 | 5130 | −36 | −33 | 86 | C |

| | | | | | | | | | | |
|---|---|---|---|---|---|---|---|---|---|---|
| ATOM | 2234 | C | ALA B | 74 | 23.427 | 23.938 | 52.003 | 1.00 | 42.80 | C |

| | | | | | | | | | | | |
|---|---|---|---|---|---|---|---|---|---|---|---|
| ANISOU | 2234 | C | ALA B | 74 | 5445 | 5453 | 5364 | −52 | 95 | −26 | C |

| | | | | | | | | | | |
|---|---|---|---|---|---|---|---|---|---|---|
| ATOM | 2235 | O | ALA B | 74 | 24.478 | 23.548 | 52.524 | 1.00 | 44.84 | O |

| | | | | | | | | | | | |
|---|---|---|---|---|---|---|---|---|---|---|---|
| ANISOU | 2235 | O | ALA B | 74 | 5616 | 5781 | 5640 | 86 | 77 | 32 | O |

| | | | | | | | | | | |
|---|---|---|---|---|---|---|---|---|---|---|
| ATOM | 2236 | CB | ALA B | 74 | 23.220 | 26.371 | 52.507 | 1.00 | 41.18 | C |

| | | | | | | | | | | | |
|---|---|---|---|---|---|---|---|---|---|---|---|
| ANISOU | 2236 | CB | ALA B | 74 | 5213 | 5260 | 5175 | −30 | −10 | 2 | C |

| | | | | | | | | | | |
|---|---|---|---|---|---|---|---|---|---|---|
| ATOM | 2237 | N | ASP B | 75 | 22.984 | 23.374 | 50.882 | 1.00 | 43.38 | N |

| | | | | | | | | | | | |
|---|---|---|---|---|---|---|---|---|---|---|---|
| ANISOU | 2237 | N | ASP B | 75 | 5569 | 5523 | 5393 | −47 | 117 | −63 | N |

| | | | | | | | | | | |
|---|---|---|---|---|---|---|---|---|---|---|
| ATOM | 2238 | CA | ASP B | 75 | 23.920 | 22.331 | 50.362 | 1.00 | 42.67 | C |

| | | | | | | | | | | | |
|---|---|---|---|---|---|---|---|---|---|---|---|
| ANISOU | 2238 | CA | ASP B | 75 | 5413 | 5411 | 5387 | −48 | 64 | 26 | C |

| | | | | | | | | | | |
|---|---|---|---|---|---|---|---|---|---|---|
| ATOM | 2239 | C | ASP B | 75 | 23.413 | 20.947 | 50.702 | 1.00 | 40.94 | C |

| | | | | | | | | | | | |
|---|---|---|---|---|---|---|---|---|---|---|---|
| ANISOU | 2239 | C | ASP B | 75 | 5140 | 5349 | 5066 | 20 | 121 | 18 | C |

| | | | | | | | | | | |
|---|---|---|---|---|---|---|---|---|---|---|
| ATOM | 2240 | O | ASP B | 75 | 23.780 | 19.968 | 50.046 | 1.00 | 41.04 | O |

| | | | | | | | | | | | |
|---|---|---|---|---|---|---|---|---|---|---|---|
| ANISOU | 2240 | O | ASP B | 75 | 5068 | 5417 | 5109 | 74 | 92 | −28 | O |

| | | | | | | | | | | |
|---|---|---|---|---|---|---|---|---|---|---|
| ATOM | 2241 | N | GLN B | 76 | 22.576 | 20.843 | 51.735 | 1.00 | 39.10 | N |

| | | | | | | | | | | | |
|---|---|---|---|---|---|---|---|---|---|---|---|
| ANISOU | 2241 | N | GLN B | 76 | 4907 | 4999 | 4949 | 51 | 47 | −5 | N |

| | | | | | | | | | | |
|---|---|---|---|---|---|---|---|---|---|---|
| ATOM | 2242 | CA | GLN B | 76 | 21.884 | 19.598 | 52.000 | 1.00 | 36.70 | C |

| | | | | | | | | | | | |
|---|---|---|---|---|---|---|---|---|---|---|---|
| ANISOU | 2242 | CA | GLN B | 76 | 4653 | 4850 | 4442 | 122 | 23 | −44 | C |

| | | | | | | | | | | |
|---|---|---|---|---|---|---|---|---|---|---|
| ATOM | 2243 | C | GLN B | 76 | 20.705 | 19.672 | 50.993 | 1.00 | 35.71 | C |

| | | | | | | | | | | | |
|---|---|---|---|---|---|---|---|---|---|---|---|
| ANISOU | 2243 | C | GLN B | 76 | 4500 | 4682 | 4386 | 58 | 117 | −28 | C |

| | | | | | | | | | | |
|---|---|---|---|---|---|---|---|---|---|---|
| ATOM | 2244 | O | GLN B | 76 | 20.273 | 20.782 | 50.645 | 1.00 | 35.70 | O |

| | | | | | | | | | | | |
|---|---|---|---|---|---|---|---|---|---|---|---|
| ANISOU | 2244 | O | GLN B | 76 | 4506 | 4670 | 4389 | 73 | 157 | −26 | O |

| | | | | | | | | | | |
|---|---|---|---|---|---|---|---|---|---|---|
| ATOM | 2245 | CB | GLN B | 76 | 21.275 | 19.429 | 53.390 | 1.00 | 36.16 | C |

| | | | | | | | | | | | |
|---|---|---|---|---|---|---|---|---|---|---|---|
| ANISOU | 2245 | CB | GLN B | 76 | 4548 | 4751 | 4439 | 72 | 19 | −11 | C |

| | | | | | | | | | | |
|---|---|---|---|---|---|---|---|---|---|---|
| ATOM | 2246 | CG | GLN B | 76 | 22.308 | 19.265 | 54.507 | 1.00 | 35.85 | C |

| | | | | | | | | | | | |
|---|---|---|---|---|---|---|---|---|---|---|---|
| ANISOU | 2246 | CG | GLN B | 76 | 4452 | 4742 | 4428 | 93 | 68 | 9 | C |

| | | | | | | | | | | |
|---|---|---|---|---|---|---|---|---|---|---|
| ATOM | 2247 | CD | GLN B | 76 | 21.613 | 18.955 | 55.829 | 1.00 | 35.73 | C |

| | | | | | | | | | | | |
|---|---|---|---|---|---|---|---|---|---|---|---|
| ANISOU | 2247 | CD | GLN B | 76 | 4666 | 4592 | 4316 | 66 | −48 | 21 | C |

| | | | | | | | | | | |
|---|---|---|---|---|---|---|---|---|---|---|
| ATOM | 2248 | OE1 | GLN B | 76 | 21.081 | 19.855 | 56.481 | 1.00 | 34.71 | O |

| | | | | | | | | | | | |
|---|---|---|---|---|---|---|---|---|---|---|---|
| ANISOU | 2248 | OE1 | GLN B | 76 | 4189 | 4657 | 4344 | 147 | 14 | 78 | O |

| | | | | | | | | | | |
|---|---|---|---|---|---|---|---|---|---|---|
| ATOM | 2249 | NE2 | GLN B | 76 | 21.633 | 17.669 | 56.182 | 1.00 | 35.30 | N |

| | | | | | | | | | | | |
|---|---|---|---|---|---|---|---|---|---|---|---|
| ANISOU | 2249 | NE2 | GLN B | 76 | 4580 | 4521 | 4309 | −50 | −84 | −22 | N |

| | | | | | | | | | | |
|---|---|---|---|---|---|---|---|---|---|---|
| ATOM | 2250 | N | THR B | 77 | 20.251 | 18.533 | 50.552 | 1.00 | 35.00 | N |

| | | | | | | | | | | | |
|---|---|---|---|---|---|---|---|---|---|---|---|
| ANISOU | 2250 | N | THR B | 77 | 4352 | 4621 | 4325 | 111 | 170 | −59 | N |

| | | | | | | | | | | |
|---|---|---|---|---|---|---|---|---|---|---|
| ATOM | 2251 | CA | THR B | 77 | 19.118 | 18.487 | 49.624 | 1.00 | 35.59 | C |

| | | | | | | | | | | | |
|---|---|---|---|---|---|---|---|---|---|---|---|
| ANISOU | 2251 | CA | THR B | 77 | 4537 | 4588 | 4398 | 78 | 43 | −34 | C |

| | | | | | | | | | | |
|---|---|---|---|---|---|---|---|---|---|---|
| ATOM | 2252 | C | THR B | 77 | 17.887 | 17.990 | 50.355 | 1.00 | 35.67 | C |

| | | | | | | | | | | | |
|---|---|---|---|---|---|---|---|---|---|---|---|
| ANISOU | 2252 | C | THR B | 77 | 4566 | 4511 | 4477 | 94 | 61 | −58 | C |

| | | | | | | | | | | |
|---|---|---|---|---|---|---|---|---|---|---|
| ATOM | 2253 | O | THR B | 77 | 18.005 | 17.366 | 51.415 | 1.00 | 35.20 | O |

| | | | | | | | | | | | |
|---|---|---|---|---|---|---|---|---|---|---|---|
| ANISOU | 2253 | O | THR B | 77 | 4453 | 4601 | 4320 | 146 | 77 | −125 | O |

| | | | | | | | | | | |
|---|---|---|---|---|---|---|---|---|---|---|
| ATOM | 2254 | CB | THR B | 77 | 19.423 | 17.436 | 48.530 | 1.00 | 36.24 | C |

| | | | | | | | | | | | |
|---|---|---|---|---|---|---|---|---|---|---|---|
| ANISOU | 2254 | CB | THR B | 77 | 4633 | 4648 | 4486 | 39 | 39 | −72 | C |

| | | | | | | | | | | |
|---|---|---|---|---|---|---|---|---|---|---|
| ATOM | 2255 | OG1 | THR B | 77 | 19.518 | 16.150 | 49.150 | 1.00 | 35.57 | O |

| | | | | | | | | | | | |
|---|---|---|---|---|---|---|---|---|---|---|---|
| ANISOU | 2255 | OG1 | THR B | 77 | 4498 | 4650 | 4369 | 101 | 88 | −112 | O |

| | | | | | | | | | | |
|---|---|---|---|---|---|---|---|---|---|---|
| ATOM | 2256 | CD2 | THR B | 77 | 20.714 | 17.763 | 47.809 | 1.00 | 36.61 | C |

| | | | | | | | | | | | |
|---|---|---|---|---|---|---|---|---|---|---|---|
| ANISOU | 2256 | CD2 | THR B | 77 | 4565 | 4790 | 4555 | 48 | 30 | −88 | C |

| | | | | | | | | | | |
|---|---|---|---|---|---|---|---|---|---|---|
| ATOM | 2257 | N | PHE B | 78 | 16.715 | 18.068 | 49.742 | 1.00 | 36.39 | N |

| | | | | | | | | | | | |
|---|---|---|---|---|---|---|---|---|---|---|---|
| ANISOU | 2257 | N | PHE B | 78 | 4685 | 4648 | 4495 | 129 | −2 | −20 | N |

| | | | | | | | | | | |
|---|---|---|---|---|---|---|---|---|---|---|
| ATOM | 2258 | CA | PHE B | 78 | 15.493 | 17.506 | 50.292 | 1.00 | 37.02 | C |

| | | | | | | | | | | | |
|---|---|---|---|---|---|---|---|---|---|---|---|
| ANISOU | 2258 | CA | PHE B | 78 | 4681 | 4770 | 4615 | 65 | 7 | −26 | C |

| | | | | | | | | | | |
|---|---|---|---|---|---|---|---|---|---|---|
| ATOM | 2259 | C | PHE B | 78 | 15.638 | 16.001 | 50.513 | 1.00 | 39.10 | C |

| | | | | | | | | | | | |
|---|---|---|---|---|---|---|---|---|---|---|---|
| ANISOU | 2259 | C | PHE B | 78 | 5054 | 4873 | 4928 | 100 | 20 | 44 | C |

| | | | | | | | | | | |
|---|---|---|---|---|---|---|---|---|---|---|
| ATOM | 2260 | O | PHE B | 78 | 15.197 | 15.402 | 51.502 | 1.00 | 38.80 | O |

| | | | | | | | | | | | |
|---|---|---|---|---|---|---|---|---|---|---|---|
| ANISOU | 2260 | O | PHE B | 78 | 4976 | 4916 | 4851 | 114 | 77 | −16 | O |

| | | | | | | | | | | |
|---|---|---|---|---|---|---|---|---|---|---|
| ATOM | 2261 | CB | PHE B | 78 | 14.450 | 17.807 | 49.222 | 1.00 | 36.04 | C |

| | | | | | | | | | | | |
|---|---|---|---|---|---|---|---|---|---|---|---|
| ANISOU | 2261 | CB | PHE B | 78 | 4715 | 4563 | 4416 | 58 | 55 | −56 | C |

| | | | | | | | | | | |
|---|---|---|---|---|---|---|---|---|---|---|
| ATOM | 2262 | CG | PHE B | 78 | 14.964 | 19.057 | 48.580 | 1.00 | 34.93 | C |

| | | | | | | | | | | | |
|---|---|---|---|---|---|---|---|---|---|---|---|
| ANISOU | 2262 | CG | PHE B | 78 | 4446 | 4572 | 4255 | 66 | 22 | −74 | C |

| | | | | | | | | | | |
|---|---|---|---|---|---|---|---|---|---|---|
| ATOM | 2263 | CD | PHE B | 78 | 16.464 | 18.800 | 48.464 | 1.00 | 36.11 | C |

| | | | | | | | | | | | |
|---|---|---|---|---|---|---|---|---|---|---|---|
| ANISOU | 2263 | CD | PHE B | 78 | 4519 | 4732 | 4469 | 124 | −26 | −47 | C |

| | | | | | | | | | | |
|---|---|---|---|---|---|---|---|---|---|---|
| ATOM | 2264 | N | GLU B | 79 | 16.292 | 15.329 | 49.551 | 1.00 | 40.36 | N |

| | | | | | | | | | | | |
|---|---|---|---|---|---|---|---|---|---|---|---|
| ANISOU | 2264 | N | GLU B | 79 | 5193 | 5200 | 4942 | 148 | −52 | 52 | N |

| | | | | | | | | | | |
|---|---|---|---|---|---|---|---|---|---|---|
| ATOM | 2265 | CA | GLU B | 79 | 16.553 | 13.892 | 49.693 | 1.00 | 42.01 | C |

| | | | | | | | | | | | |
|---|---|---|---|---|---|---|---|---|---|---|---|
| ANISOU | 2265 | CA | GLU B | 79 | 5425 | 5273 | 5265 | 121 | −12 | −10 | C |

| | | | | | | | | | | |
|---|---|---|---|---|---|---|---|---|---|---|
| ATOM | 2266 | C | GLU B | 79 | 17.416 | 13.645 | 50.928 | 1.00 | 41.29 | C |

| | | | | | | | | | | | |
|---|---|---|---|---|---|---|---|---|---|---|---|
| ANISOU | 2266 | C | GLU B | 79 | 5264 | 5245 | 5180 | 136 | 77 | −70 | C |

| | | | | | | | | | | |
|---|---|---|---|---|---|---|---|---|---|---|
| ATOM | 2267 | O | GLU B | 79 | 17.142 | 12.732 | 51.718 | 1.00 | 41.38 | O |

| | | | | | | | | | | | |
|---|---|---|---|---|---|---|---|---|---|---|---|
| ANISOU | 2267 | O | GLU B | 79 | 5311 | 5316 | 5095 | 188 | 71 | −61 | O |

| | | | | | | | | | | |
|---|---|---|---|---|---|---|---|---|---|---|
| ATOM | 2268 | CB | GLU B | 79 | 17.197 | 13.360 | 48.410 | 1.00 | 43.78 | C |

| | | | | | | | | | | | |
|---|---|---|---|---|---|---|---|---|---|---|---|
| ANISOU | 2268 | CB | GLU B | 79 | 5575 | 5681 | 5380 | 98 | 56 | −94 | C |

| | | | | | | | | | | |
|---|---|---|---|---|---|---|---|---|---|---|
| ATOM | 2269 | CG | GLU B | 79 | 17.521 | 11.876 | 48.491 | 1.00 | 46.12 | C |

| | | | | | | | | | | | |
|---|---|---|---|---|---|---|---|---|---|---|---|
| ANISOU | 2269 | CG | GLU B | 79 | 5937 | 5750 | 5836 | 60 | −7 | −31 | C |

| | | | | | | | | | | |
|---|---|---|---|---|---|---|---|---|---|---|
| ATOM | 2270 | CD | GLU B | 79 | 18.321 | 11.381 | 47.292 | 1.00 | 47.70 | C |

| | | | | | | | | | | | |
|---|---|---|---|---|---|---|---|---|---|---|---|
| ANISOU | 2270 | CD | GLU B | 79 | 6102 | 6017 | 6006 | 83 | 100 | −94 | C |

| | | | | | | | | | | |
|---|---|---|---|---|---|---|---|---|---|---|
| ATOM | 2271 | OE1 | GLU B | 79 | 19.353 | 11.964 | 46.902 | 1.00 | 47.99 | O |

| | | | | | | | | | | | |
|---|---|---|---|---|---|---|---|---|---|---|---|
| ANISOU | 2271 | OE1 | GLU B | 79 | 6138 | 6021 | 6076 | 100 | 147 | −99 | O |

| | | | | | | | | | | |
|---|---|---|---|---|---|---|---|---|---|---|
| ATOM | 2272 | OE2 | GLU B | 79 | 17.879 | 10.357 | 46.742 | 1.00 | 48.39 | O |

| | | | | | | | | | | | |
|---|---|---|---|---|---|---|---|---|---|---|---|
| ANISOU | 2272 | OE2 | GLU B | 79 | 6172 | 6163 | 6052 | 17 | 62 | −147 | O |

| | | | | | | | | | | |
|---|---|---|---|---|---|---|---|---|---|---|
| ATOM | 2273 | N | ASP B | 80 | 18.438 | 14.456 | 51.169 | 1.00 | 40.72 | N |

| | | | | | | | | | | | |
|---|---|---|---|---|---|---|---|---|---|---|---|
| ANISOU | 2273 | N | ASP B | 80 | 5242 | 5181 | 5047 | 177 | 86 | −87 | N |

| | | | | | | | | | | |
|---|---|---|---|---|---|---|---|---|---|---|
| ATOM | 2274 | CA | ASP B | 80 | 19.270 | 14.336 | 52.362 | 1.00 | 40.90 | C |

| | | | | | | | | | | | |
|---|---|---|---|---|---|---|---|---|---|---|---|
| ANISOU | 2274 | CA | ASP B | 80 | 5181 | 5183 | 5177 | 92 | 27 | −13 | C |

| | | | | | | | | | | |
|---|---|---|---|---|---|---|---|---|---|---|
| ATOM | 2275 | C | ASP B | 80 | 18.447 | 14.311 | 53.655 | 1.00 | 41.39 | C |

| | | | | | | | | | | | |
|---|---|---|---|---|---|---|---|---|---|---|---|
| ANISOU | 2275 | C | ASP B | 80 | 5277 | 5258 | 5190 | 120 | 47 | −7 | C |

| | | | | | | | | | | |
|---|---|---|---|---|---|---|---|---|---|---|
| ATOM | 2276 | O | ASP B | 80 | 18.662 | 13.494 | 54.561 | 1.00 | 41.76 | O |

| | | | | | | | | | | | |
|---|---|---|---|---|---|---|---|---|---|---|---|
| ANISOU | 2276 | O | ASP B | 80 | 5337 | 5353 | 5177 | 202 | 6 | 4 | O |

| | | | | | | | | | | |
|---|---|---|---|---|---|---|---|---|---|---|
| ATOM | 2277 | CB | ASP B | 80 | 20.255 | 15.505 | 52.434 | 1.00 | 40.50 | C |

| | | | | | | | | | | | |
|---|---|---|---|---|---|---|---|---|---|---|---|
| ANISOU | 2277 | CB | ASP B | 80 | 5105 | 5202 | 5082 | 79 | 58 | −7 | C |

| | | | | | | | | | | |
|---|---|---|---|---|---|---|---|---|---|---|
| ATOM | 2278 | CG | ASP B | 80 | 21.458 | 15.369 | 51.531 | 1.00 | 39.97 | C |

| | | | | | | | | | | | |
|---|---|---|---|---|---|---|---|---|---|---|---|
| ANISOU | 2278 | CG | ASP B | 80 | 5073 | 5096 | 5017 | 71 | 11 | −31 | C |

| | | | | | | | | | | |
|---|---|---|---|---|---|---|---|---|---|---|
| ATOM | 2279 | OD1 | ASP B | 80 | 21.891 | 14.227 | 51.288 | 1.00 | 39.37 | O |

| | | | | | | | | | | | |
|---|---|---|---|---|---|---|---|---|---|---|---|
| ANISOU | 2279 | OD1 | ASP B | 80 | 4979 | 5115 | 4866 | 95 | 39 | −8 | O |

| | | | | | | | | | | |
|---|---|---|---|---|---|---|---|---|---|---|
| ATOM | 2280 | OD2 | ASP B | 80 | 22.002 | 16.390 | 51.058 | 1.00 | 40.19 | O |

| | | | | | | | | | | | |
|---|---|---|---|---|---|---|---|---|---|---|---|
| ANISOU | 2280 | OD2 | ASP B | 80 | 5059 | 5128 | 5083 | 139 | 87 | −18 | O |

| | | | | | | | | | | |
|---|---|---|---|---|---|---|---|---|---|---|
| ATOM | 2281 | N | LEU B | 81 | 17.452 | 15.193 | 53.752 | 1.00 | 40.72 | N |

| | | | | | | | | | | | |
|---|---|---|---|---|---|---|---|---|---|---|---|
| ANISOU | 2281 | N | LEU B | 81 | 5205 | 5182 | 5085 | 94 | 35 | 6 | N |

| | | | | | | | | | | |
|---|---|---|---|---|---|---|---|---|---|---|
| ATOM | 2282 | CA | LEU B | 81 | 16.604 | 15.338 | 54.931 | 1.00 | 39.97 | C |

| | | | | | | | | | | | |
|---|---|---|---|---|---|---|---|---|---|---|---|
| ANISOU | 2282 | CA | LEU B | 81 | 5058 | 5090 | 5039 | 54 | −28 | 48 | C |

| | | | | | | | | | | |
|---|---|---|---|---|---|---|---|---|---|---|
| ATOM | 2283 | C | LEU B | 81 | 15.333 | 14.528 | 54.960 | 1.00 | 39.64 | C |

| | | | | | | | | | | | |
|---|---|---|---|---|---|---|---|---|---|---|---|
| ANISOU | 2283 | C | LEU B | 81 | 5037 | 5034 | 4991 | 88 | 4 | 70 | C |

| | | | | | | | | | | |
|---|---|---|---|---|---|---|---|---|---|---|
| ATOM | 2284 | O | LEU B | 81 | 14.504 | 14.683 | 55.869 | 1.00 | 38.32 | O |

| | | | | | | | | | | | |
|---|---|---|---|---|---|---|---|---|---|---|---|
| ANISOU | 2284 | O | LEU B | 81 | 4836 | 4787 | 4938 | −92 | 92 | 109 | O |

| | | | | | | | | | | |
|---|---|---|---|---|---|---|---|---|---|---|
| ATOM | 2285 | CB | LEU B | 81 | 16.325 | 16.856 | 55.064 | 1.00 | 39.74 | C |

| | | | | | | | | | | | |
|---|---|---|---|---|---|---|---|---|---|---|---|
| ANISOU | 2285 | CB | LEU B | 81 | 5066 | 5109 | 4926 | 13 | 51 | −42 | C |

| | | | | | | | | | | |
|---|---|---|---|---|---|---|---|---|---|---|
| ATOM | 2286 | CG | LEU B | 81 | 17.616 | 17.650 | 55.337 | 1.00 | 39.96 | C |

| | | | | | | | | | | | |
|---|---|---|---|---|---|---|---|---|---|---|---|
| ANISOU | 2286 | CG | LEU B | 81 | 5045 | 5143 | 4994 | −2 | 59 | 18 | C |

| | | | | | | | | | | |
|---|---|---|---|---|---|---|---|---|---|---|
| ATOM | 2287 | CD1 | LEU B | 81 | 17.503 | 19.114 | 54.972 | 1.00 | 40.36 | C |

| | | | | | | | | | | | |
|---|---|---|---|---|---|---|---|---|---|---|---|
| ANISOU | 2287 | CD1 | LEU B | 81 | 5140 | 5111 | 5084 | 44 | 46 | −41 | C |

| | | | | | | | | | | |
|---|---|---|---|---|---|---|---|---|---|---|
| ATOM | 2288 | CD2 | LEU B | 81 | 17.999 | 17.511 | 56.809 | 1.00 | 40.56 | C |

| | | | | | | | | | | | |
|---|---|---|---|---|---|---|---|---|---|---|---|
| ANISOU | 2288 | CD2 | LEU B | 81 | 5128 | 5270 | 5015 | −23 | 15 | −53 | C |

| | | | | | | | | | | |
|---|---|---|---|---|---|---|---|---|---|---|
| ATOM | 2289 | N | ASP B | 82 | 15.093 | 13.654 | 53.978 | 1.00 | 40.45 | N |

| | | | | | | | | | | | |
|---|---|---|---|---|---|---|---|---|---|---|---|
| ANISOU | 2289 | N | ASP B | 82 | 5185 | 5067 | 5115 | 74 | −35 | 28 | N |

| | | | | | | | | | | |
|---|---|---|---|---|---|---|---|---|---|---|
| ATOM | 2290 | CA | ASP B | 82 | 13.890 | 12.823 | 53.960 | 1.00 | 41.27 | C |

| | | | | | | | | | | | |
|---|---|---|---|---|---|---|---|---|---|---|---|
| ANISOU | 2290 | CA | ASP B | 82 | 5147 | 5240 | 5295 | 55 | −43 | −68 | C |

| | | | | | | | | | | |
|---|---|---|---|---|---|---|---|---|---|---|
| ATOM | 2291 | C | ASP B | 82 | 12.610 | 13.641 | 53.846 | 1.00 | 38.94 | C |

| | | | | | | | | | | | |
|---|---|---|---|---|---|---|---|---|---|---|---|
| ANISOU | 2291 | C | ASP B | 82 | 5091 | 4877 | 4825 | −39 | −5 | 1 | C |

| | | | | | | | | | | |
|---|---|---|---|---|---|---|---|---|---|---|
| ATOM | 2292 | O | ASP B | 82 | 11.569 | 13.350 | 54.437 | 1.00 | 38.51 | O |

| | | | | | | | | | | | |
|---|---|---|---|---|---|---|---|---|---|---|---|
| ANISOU | 2292 | O | ASP B | 82 | 5005 | 4892 | 4733 | −45 | −35 | −82 | O |

| | | | | | | | | | | |
|---|---|---|---|---|---|---|---|---|---|---|
| ATOM | 2293 | CB | ASP B | 82 | 13.903 | 11.949 | 55.217 | 1.00 | 44.41 | C |

| | | | | | | | | | | | |
|---|---|---|---|---|---|---|---|---|---|---|---|
| ANISOU | 2293 | CB | ASP B | 82 | 5763 | 5606 | 5506 | 71 | −26 | 88 | C |

| | | | | | | | | | | |
|---|---|---|---|---|---|---|---|---|---|---|
| ATOM | 2294 | CG | ASP B | 82 | 12.787 | 10.949 | 55.365 | 1.00 | 47.24 | C |

| | | | | | | | | | | | |
|---|---|---|---|---|---|---|---|---|---|---|---|
| ANISOU | 2294 | CG | ASP B | 82 | 5952 | 5986 | 6012 | −102 | 2 | 16 | C |

| | | | | | | | | | | |
|---|---|---|---|---|---|---|---|---|---|---|
| ATOM | 2295 | OD1 | ASP B | 82 | 12.202 | 10.534 | 54.333 | 1.00 | 48.41 | O |

| | | | | | | | | | | | |
|---|---|---|---|---|---|---|---|---|---|---|---|
| ANISOU | 2295 | OD1 | ASP B | 82 | 6186 | 6236 | 5971 | −45 | −5 | −23 | O |

| | | | | | | | | | | |
|---|---|---|---|---|---|---|---|---|---|---|
| ATOM | 2296 | OD2 | ASP B | 82 | 12.442 | 10.561 | 56.505 | 1.00 | 48.88 | O |

| | | | | | | | | | | | |
|---|---|---|---|---|---|---|---|---|---|---|---|
| ANISOU | 2296 | OD2 | ASP B | 82 | 6341 | 6238 | 5993 | −28 | 15 | 14 | O |

| | | | | | | | | | | |
|---|---|---|---|---|---|---|---|---|---|---|
| ATOM | 2297 | N | MET B | 83 | 12.661 | 14.741 | 53.092 | 1.00 | 36.91 | N |

| | | | | | | | | | | | |
|---|---|---|---|---|---|---|---|---|---|---|---|
| ANISOU | 2297 | N | MET B | 83 | 4787 | 4781 | 4457 | 75 | −6 | −95 | N |

| | | | | | | | | | | |
|---|---|---|---|---|---|---|---|---|---|---|
| ATOM | 2298 | CA | MET B | 83 | 11.470 | 15.569 | 52.898 | 1.00 | 35.33 | C |

| | | | | | | | | | | | |
|---|---|---|---|---|---|---|---|---|---|---|---|
| ANISOU | 2298 | CA | MET B | 83 | 4651 | 4482 | 4292 | −7 | 90 | −43 | C |

| | | | | | | | | | | |
|---|---|---|---|---|---|---|---|---|---|---|
| ATOM | 2299 | C | MET B | 83 | 10.453 | 14.882 | 51.998 | 1.00 | 36.41 | C |

| | | | | | | | | | | | |
|---|---|---|---|---|---|---|---|---|---|---|---|
| ANISOU | 2299 | C | MET B | 83 | 4689 | 4652 | 4493 | 29 | 10 | −71 | C |

| | | | | | | | | | | |
|---|---|---|---|---|---|---|---|---|---|---|
| ATOM | 2300 | O | MET B | 83 | 10.818 | 14.005 | 51.200 | 1.00 | 37.09 | O |

| | | | | | | | | | | | |
|---|---|---|---|---|---|---|---|---|---|---|---|
| ANISOU | 2300 | O | MET B | 83 | 4971 | 4677 | 4445 | 1 | 1 | −132 | O |

| | | | | | | | | | | |
|---|---|---|---|---|---|---|---|---|---|---|
| ATOM | 2301 | CB | MET B | 83 | 11.882 | 16.932 | 52.333 | 1.00 | 33.81 | C |

| | | | | | | | | | | | |
|---|---|---|---|---|---|---|---|---|---|---|---|
| ANISOU | 2301 | CB | MET B | 83 | 4343 | 4369 | 4133 | 84 | −44 | −86 | C |

| | | | | | | | | | | |
|---|---|---|---|---|---|---|---|---|---|---|
| ATOM | 2302 | CG | MET B | 83 | 12.881 | 17.677 | 53.217 | 1.00 | 31.70 | C |

| | | | | | | | | | | | |
|---|---|---|---|---|---|---|---|---|---|---|---|
| ANISOU | 2302 | CG | MET B | 83 | 4195 | 4009 | 3839 | 140 | 80 | −29 | C |

| | | | | | | | | | | |
|---|---|---|---|---|---|---|---|---|---|---|
| ATOM | 2303 | SD | MET B | 83 | 13.259 | 19.326 | 52.606 | 1.00 | 31.16 | 5 |

| | | | | | | | | | | | |
|---|---|---|---|---|---|---|---|---|---|---|---|
| ANISOU | 2303 | SD | MET B | 83 | 4237 | 3999 | 3603 | 191 | 77 | −73 | 5 |

| | | | | | | | | | | |
|---|---|---|---|---|---|---|---|---|---|---|
| ATOM | 2304 | CE | MET B | 83 | 13.994 | 20.038 | 54.074 | 1.00 | 31.37 | C |

| | | | | | | | | | | | |
|---|---|---|---|---|---|---|---|---|---|---|---|
| ANISOU | 2304 | CE | MET B | 83 | 4141 | 4011 | 3766 | 96 | 11 | −50 | C |

| | | | | | | | | | | |
|---|---|---|---|---|---|---|---|---|---|---|
| ATOM | 2305 | N | GLU B | 84 | 9.183 | 15.224 | 52.135 | 1.00 | 36.04 | N |

| | | | | | | | | | | | |
|---|---|---|---|---|---|---|---|---|---|---|---|
| ANISOU | 2305 | N | GLU B | 84 | 4662 | 4593 | 4439 | −52 | −18 | −109 | N |

| | | | | | | | | | | |
|---|---|---|---|---|---|---|---|---|---|---|
| ATOM | 2306 | CA | GLU B | 84 | 8.084 | 14.752 | 51.318 | 1.00 | 35.35 | C |

| | | | | | | | | | | | |
|---|---|---|---|---|---|---|---|---|---|---|---|
| ANISOU | 2306 | CA | GLU B | 84 | 4504 | 4572 | 4356 | 29 | −38 | −2 | C |

| | | | | | | | | | | |
|---|---|---|---|---|---|---|---|---|---|---|
| ATOM | 2307 | C | GLU B | 84 | 7.364 | 15.973 | 50.736 | 1.00 | 35.04 | C |

| | | | | | | | | | | | |
|---|---|---|---|---|---|---|---|---|---|---|---|
| ANISOU | 2307 | C | GLU B | 84 | 4491 | 4498 | 4322 | −60 | −45 | −51 | C |

| | | | | | | | | | | |
|---|---|---|---|---|---|---|---|---|---|---|
| ATOM | 2308 | O | GLU B | 84 | 7.447 | 17.076 | 51.253 | 1.00 | 33.52 | O |

| | | | | | | | | | | | |
|---|---|---|---|---|---|---|---|---|---|---|---|
| ANISOU | 2308 | O | GLU B | 84 | 4285 | 4465 | 3988 | 54 | −109 | 42 | O |

| | | | | | | | | | | |
|---|---|---|---|---|---|---|---|---|---|---|
| ATOM | 2309 | CB | GLU B | 84 | 7.055 | 13.905 | 52.049 | 1.00 | 35.95 | C |

| | | | | | | | | | | | |
|---|---|---|---|---|---|---|---|---|---|---|---|
| ANISOU | 2309 | CB | GLU B | 84 | 4539 | 4620 | 4501 | −45 | −58 | −23 | C |

| | | | | | | | | | | |
|---|---|---|---|---|---|---|---|---|---|---|
| ATOM | 2310 | N | ASP B | 85 | 6.648 | 15.721 | 49.639 | 1.00 | 34.60 | N |

| | | | | | | | | | | | |
|---|---|---|---|---|---|---|---|---|---|---|---|
| ANISOU | 2310 | N | ASP B | 85 | 4265 | 4627 | 4253 | −3 | 2 | −18 | N |

| | | | | | | | | | | |
|---|---|---|---|---|---|---|---|---|---|---|
| ATOM | 2311 | CA | ASP B | 85 | 5.945 | 16.778 | 48.935 | 1.00 | 35.80 | C |

| | | | | | | | | | | | |
|---|---|---|---|---|---|---|---|---|---|---|---|
| ANISOU | 2311 | CA | ASP B | 85 | 4574 | 4602 | 4427 | 14 | −8 | −40 | C |

| | | | | | | | | | | |
|---|---|---|---|---|---|---|---|---|---|---|
| ATOM | 2312 | C | ASP B | 85 | 5.078 | 17.603 | 49.870 | 1.00 | 34.39 | C |

| | | | | | | | | | | | |
|---|---|---|---|---|---|---|---|---|---|---|---|
| ANISOU | 2312 | C | ASP B | 85 | 4290 | 4555 | 4222 | −54 | −43 | 26 | C |

| | | | | | | | | | | |
|---|---|---|---|---|---|---|---|---|---|---|
| ATOM | 2313 | O | ASP B | 85 | 4.333 | 17.101 | 50.719 | 1.00 | 33.99 | O |

| | | | | | | | | | | | |
|---|---|---|---|---|---|---|---|---|---|---|---|
| ANISOU | 2313 | O | ASP B | 85 | 4317 | 4566 | 4030 | −146 | −69 | −109 | O |

| | | | | | | | | | | |
|---|---|---|---|---|---|---|---|---|---|---|
| ATOM | 2314 | CB | ASP B | 85 | 5.139 | 16.090 | 47.819 | 1.00 | 37.12 | C |

| | | | | | | | | | | | |
|---|---|---|---|---|---|---|---|---|---|---|---|
| ANISOU | 2314 | CB | ASP B | 85 | 4608 | 4868 | 4626 | −82 | −71 | −81 | C |

| | | | | | | | | | | |
|---|---|---|---|---|---|---|---|---|---|---|
| ATOM | 2315 | CG | ASP B | 85 | 4.674 | 17.003 | 46.726 | 1.00 | 38.28 | C |

| | | | | | | | | | | | |
|---|---|---|---|---|---|---|---|---|---|---|---|
| ANISOU | 2315 | CG | ASP B | 85 | 4901 | 4993 | 4651 | −26 | −13 | −12 | C |

| | | | | | | | | | | |
|---|---|---|---|---|---|---|---|---|---|---|
| ATOM | 2316 | OD1 | ASP B | 85 | 5.322 | 18.024 | 46.401 | 1.00 | 37.59 | O |

| | | | | | | | | | | | |
|---|---|---|---|---|---|---|---|---|---|---|---|
| ANISOU | 2316 | OD1 | ASP B | 85 | 4871 | 5044 | 4569 | −58 | −83 | −118 | O |

| | | | | | | | | | | |
|---|---|---|---|---|---|---|---|---|---|---|
| ATOM | 2317 | OD2 | ASP B | 85 | 3.599 | 16.684 | 46.158 | 1.00 | 39.56 | O |

| | | | | | | | | | | | |
|---|---|---|---|---|---|---|---|---|---|---|---|
| ANISOU | 2317 | OD2 | ASP B | 85 | 4901 | 5201 | 4928 | −143 | 13 | −79 | O |

| | | | | | | | | | | |
|---|---|---|---|---|---|---|---|---|---|---|
| ATOM | 2318 | N | ASN B | 86 | 5.200 | 18.915 | 49.750 | 1.00 | 33.19 | N |

| | | | | | | | | | | | |
|---|---|---|---|---|---|---|---|---|---|---|---|
| ANISOU | 2318 | N | ASN B | 86 | 4126 | 4521 | 3966 | −24 | −112 | −63 | N |

| | | | | | | | | | | |
|---|---|---|---|---|---|---|---|---|---|---|
| ATOM | 2319 | CA | ASN B | 86 | 4.486 | 19.959 | 50.444 | 1.00 | 33.64 | C |

| | | | | | | | | | | | |
|---|---|---|---|---|---|---|---|---|---|---|---|
| ANISOU | 2319 | CA | ASN B | 86 | 4286 | 4460 | 4035 | −3 | −110 | −50 | C |

| | | | | | | | | | | |
|---|---|---|---|---|---|---|---|---|---|---|
| ATOM | 2320 | C | ASN B | 86 | 4.971 | 20.168 | 51.884 | 1.00 | 32.91 | C |

| | | | | | | | | | | | |
|---|---|---|---|---|---|---|---|---|---|---|---|
| ANISOU | 2320 | C | ASN B | 86 | 4150 | 4355 | 3998 | 33 | −46 | −96 | C |

| | | | | | | | | | | |
|---|---|---|---|---|---|---|---|---|---|---|
| ATOM | 2321 | O | ASN B | 86 | 4.292 | 20.835 | 52.672 | 1.00 | 31.61 | O |

| | | | | | | | | | | | |
|---|---|---|---|---|---|---|---|---|---|---|---|
| ANISOU | 2321 | O | ASN B | 86 | 3942 | 4476 | 3592 | −18 | −182 | −83 | O |

| | | | | | | | | | | |
|---|---|---|---|---|---|---|---|---|---|---|
| ATOM | 2322 | CB | ASN B | 86 | 2.966 | 19.715 | 50.429 | 1.00 | 35.28 | C |

| | | | | | | | | | | | |
|---|---|---|---|---|---|---|---|---|---|---|---|
| ANISOU | 2322 | CB | ASN B | 86 | 4367 | 4624 | 4415 | 1 | −63 | −2 | C |

| | | | | | | | | | | |
|---|---|---|---|---|---|---|---|---|---|---|
| ATOM | 2323 | CG | ASN B | 86 | 2.442 | 19.686 | 48.992 | 1.00 | 36.76 | C |

| | | | | | | | | | | | |
|---|---|---|---|---|---|---|---|---|---|---|---|
| ANISOU | 2323 | CG | ASN B | 86 | 4645 | 4833 | 4487 | −8 | −96 | −37 | C |

| | | | | | | | | | | |
|---|---|---|---|---|---|---|---|---|---|---|
| ATOM | 2324 | OD1 | ASN B | 86 | 2.767 | 20.542 | 48.182 | 1.00 | 37.26 | O |

| | | | | | | | | | | | |
|---|---|---|---|---|---|---|---|---|---|---|---|
| ANISOU | 2324 | OD1 | ASN B | 86 | 4795 | 4929 | 4432 | 38 | −42 | −24 | O |

| | | | | | | | | | | |
|---|---|---|---|---|---|---|---|---|---|---|
| ATOM | 2325 | ND2 | ASN B | 86 | 1.642 | 18.663 | 48.737 | 1.00 | 37.64 | N |

| | | | | | | | | | | | |
|---|---|---|---|---|---|---|---|---|---|---|---|
| ANISOU | 2325 | ND2 | ASN B | 86 | 4948 | 4773 | 4579 | −50 | −59 | 2 | N |

| | | | | | | | | | | |
|---|---|---|---|---|---|---|---|---|---|---|
| ATOM | 2326 | N | ASP B | 87 | 6.158 | 19.644 | 52.196 | 1.00 | 32.15 | N |

| | | | | | | | | | | | |
|---|---|---|---|---|---|---|---|---|---|---|---|
| ANISOU | 2326 | N | ASP B | 87 | 4132 | 4238 | 3843 | −31 | −77 | −18 | N |

| | | | | | | | | | | |
|---|---|---|---|---|---|---|---|---|---|---|
| ATOM | 2327 | CA | ASP B | 87 | 6.704 | 19.866 | 53.527 | 1.00 | 31.06 | C |

| | | | | | | | | | | | |
|---|---|---|---|---|---|---|---|---|---|---|---|
| ANISOU | 2327 | CA | ASP B | 87 | 4030 | 3998 | 3774 | −112 | 20 | −75 | C |

| | | | | | | | | | | |
|---|---|---|---|---|---|---|---|---|---|---|
| ATOM | 2328 | C | ASP B | 87 | 7.021 | 21.344 | 53.685 | 1.00 | 30.65 | C |

| | | | | | | | | | | | |
|---|---|---|---|---|---|---|---|---|---|---|---|
| ANISOU | 2328 | C | ASP B | 87 | 4040 | 3998 | 3606 | −34 | −20 | −63 | C |

| | | | | | | | | | | |
|---|---|---|---|---|---|---|---|---|---|---|
| ATOM | 2329 | O | ASP B | 87 | 7.159 | 22.090 | 52.725 | 1.00 | 29.16 | O |

| | | | | | | | | | | | |
|---|---|---|---|---|---|---|---|---|---|---|---|
| ANISOU | 2329 | O | ASP B | 87 | 3780 | 3974 | 3324 | −105 | −114 | −234 | O |

| | | | | | | | | | | |
|---|---|---|---|---|---|---|---|---|---|---|
| ATOM | 2330 | CB | ASP B | 87 | 7.928 | 19.025 | 53.815 | 1.00 | 31.79 | C |

| | | | | | | | | | | | |
|---|---|---|---|---|---|---|---|---|---|---|---|
| ANISOU | 2330 | CB | ASP B | 87 | 4130 | 4088 | 3861 | −62 | −38 | 16 | C |

| | | | | | | | | | | |
|---|---|---|---|---|---|---|---|---|---|---|
| ATOM | 2331 | CG | ASP B | 87 | 7.537 | 17.641 | 54.327 | 1.00 | 32.05 | C |

| | | | | | | | | | | | |
|---|---|---|---|---|---|---|---|---|---|---|---|
| ANISOU | 2331 | CG | ASP B | 87 | 4249 | 4050 | 3879 | −68 | −87 | −69 | C |

| | | | | | | | | | | |
|---|---|---|---|---|---|---|---|---|---|---|
| ATOM | 2332 | OD1 | ASP B | 87 | 6.361 | 17.459 | 54.698 | 1.00 | 33.61 | O |

| | | | | | | | | | | | |
|---|---|---|---|---|---|---|---|---|---|---|---|
| ANISOU | 2332 | OD1 | ASP B | 87 | 4358 | 4432 | 3980 | −148 | 14 | −89 | O |

| | | | | | | | | | | |
|---|---|---|---|---|---|---|---|---|---|---|
| ATOM | 2333 | OD2 | ASP B | 87 | 8.421 | 16.783 | 54.313 | 1.00 | 32.01 | O |

| | | | | | | | | | | | |
|---|---|---|---|---|---|---|---|---|---|---|---|
| ANISOU | 2333 | OD2 | ASP B | 87 | 4192 | 4111 | 3859 | −4 | −251 | −30 | O |

| | | | | | | | | | | |
|---|---|---|---|---|---|---|---|---|---|---|
| ATOM | 2334 | N | ILE B | 88 | 7.179 | 21.725 | 54.967 | 1.00 | 29.57 | N |

| | | | | | | | | | | | |
|---|---|---|---|---|---|---|---|---|---|---|---|
| ANISOU | 2334 | N | ILE B | 88 | 3743 | 3903 | 3590 | −9 | 58 | −186 | N |

| | | | | | | | | | | |
|---|---|---|---|---|---|---|---|---|---|---|
| ATOM | 2335 | CA | ILE B | 88 | 7.429 | 23.118 | 55.277 | 1.00 | 28.05 | C |

| | | | | | | | | | | | |
|---|---|---|---|---|---|---|---|---|---|---|---|
| ANISOU | 2335 | CA | ILE B | 88 | 3481 | 3766 | 3412 | −14 | 133 | 39 | C |

| | | | | | | | | | | |
|---|---|---|---|---|---|---|---|---|---|---|
| ATOM | 2336 | C | ILE B | 88 | 8.797 | 23.270 | 55.931 | 1.00 | 27.26 | C |

| | | | | | | | | | | | |
|---|---|---|---|---|---|---|---|---|---|---|---|
| ANISOU | 2336 | C | ILE B | 88 | 3530 | 3525 | 3302 | 14 | 67 | −88 | C |

| | | | | | | | | | | |
|---|---|---|---|---|---|---|---|---|---|---|
| ATOM | 2337 | O | ILE B | 88 | 9.174 | 22.471 | 56.778 | 1.00 | 28.03 | O |

| | | | | | | | | | | | |
|---|---|---|---|---|---|---|---|---|---|---|---|
| ANISOU | 2337 | O | ILE B | 88 | 3315 | 3848 | 3486 | 117 | 212 | 115 | O |

| | | | | | | | | | | |
|---|---|---|---|---|---|---|---|---|---|---|
| ATOM | 2338 | CB | ILE B | 88 | 6.396 | 23.659 | 56.288 | 1.00 | 28.41 | C |

| | | | | | | | | | | | |
|---|---|---|---|---|---|---|---|---|---|---|---|
| ANISOU | 2338 | CB | ILE B | 88 | 3428 | 3859 | 3507 | 42 | 99 | −37 | C |

| | | | | | | | | | | |
|---|---|---|---|---|---|---|---|---|---|---|
| ATOM | 2339 | CG1 | ILE B | 88 | 4.990 | 23.528 | 55.688 | 1.00 | 28.52 | C |

| | | | | | | | | | | | |
|---|---|---|---|---|---|---|---|---|---|---|---|
| ANISOU | 2339 | CG1 | ILE B | 88 | 3543 | 3988 | 3307 | −27 | 23 | −25 | C |

| | | | | | | | | | | |
|---|---|---|---|---|---|---|---|---|---|---|
| ATOM | 2340 | CG2 | ILE B | 88 | 6.675 | 25.103 | 56.656 | 1.00 | 28.14 | C |

| | | | | | | | | | | | |
|---|---|---|---|---|---|---|---|---|---|---|---|
| ANISOU | 2340 | CG2 | ILE B | 88 | 3510 | 3821 | 3361 | 6 | 127 | 43 | C |

| | | | | | | | | | | |
|---|---|---|---|---|---|---|---|---|---|---|
| ATOM | 2341 | CD1 | ILE B | 88 | 3.877 | 23.820 | 56.676 | 1.00 | 29.56 | C |

| | | | | | | | | | | | |
|---|---|---|---|---|---|---|---|---|---|---|---|
| ANISOU | 2341 | CD1 | ILE B | 88 | 3529 | 4144 | 3559 | 69 | 95 | −17 | C |

| | | | | | | | | | | |
|---|---|---|---|---|---|---|---|---|---|---|
| ATOM | 2342 | N | ILE B | 89 | 9.483 | 24.324 | 55.524 | 1.00 | 26.67 | N |

| | | | | | | | | | | | |
|---|---|---|---|---|---|---|---|---|---|---|---|
| ANISOU | 2342 | N | ILE B | 89 | 3350 | 3743 | 3041 | 77 | 156 | 60 | N |

| | | | | | | | | | | |
|---|---|---|---|---|---|---|---|---|---|---|
| ATOM | 2343 | CA | ILE B | 89 | 10.741 | 24.724 | 56.107 | 1.00 | 26.26 | C |

| | | | | | | | | | | | |
|---|---|---|---|---|---|---|---|---|---|---|---|
| ANISOU | 2343 | CA | ILE B | 89 | 3344 | 3526 | 3106 | −45 | 110 | −24 | C |

| | | | | | | | | | | |
|---|---|---|---|---|---|---|---|---|---|---|
| ATOM | 2344 | C | ILE B | 89 | 10.468 | 25.988 | 56.897 | 1.00 | 26.67 | C |

| | | | | | | | | | | | |
|---|---|---|---|---|---|---|---|---|---|---|---|
| ANISOU | 2344 | C | ILE B | 89 | 3488 | 3568 | 3080 | 26 | 4 | 1 | C |

| | | | | | | | | | | |
|---|---|---|---|---|---|---|---|---|---|---|
| ATOM | 2345 | O | ILE B | 89 | 9.939 | 26.938 | 56.324 | 1.00 | 27.18 | O |

| | | | | | | | | | | | |
|---|---|---|---|---|---|---|---|---|---|---|---|
| ANISOU | 2345 | O | ILE B | 89 | 3685 | 3667 | 2977 | 124 | −46 | −68 | O |

| | | | | | | | | | | |
|---|---|---|---|---|---|---|---|---|---|---|
| ATOM | 2346 | CB | ILE B | 89 | 11.821 | 24.953 | 55.026 | 1.00 | 26.40 | C |

| | | | | | | | | | | | |
|---|---|---|---|---|---|---|---|---|---|---|---|
| ANISOU | 2346 | CB | ILE B | 89 | 3318 | 3507 | 3205 | −31 | 57 | −9 | C |

| | | | | | | | | | | |
|---|---|---|---|---|---|---|---|---|---|---|
| ATOM | 2347 | CG1 | ILE B | 89 | 12.107 | 23.586 | 54.379 | 1.00 | 26.98 | C |

| | | | | | | | | | | | |
|---|---|---|---|---|---|---|---|---|---|---|---|
| ANISOU | 2347 | CG1 | ILE B | 89 | 3411 | 3604 | 3237 | 46 | 116 | −72 | C |

| | | | | | | | | | | |
|---|---|---|---|---|---|---|---|---|---|---|
| ATOM | 2348 | CG2 | ILE B | 89 | 13.054 | 25.557 | 55.646 | 1.00 | 24.42 | C |

| | | | | | | | | | | | |
|---|---|---|---|---|---|---|---|---|---|---|---|
| ANISOU | 2348 | CG2 | ILE B | 89 | 3131 | 3182 | 2964 | 51 | 97 | −16 | C |

| | | | | | | | | | | |
|---|---|---|---|---|---|---|---|---|---|---|
| ATOM | 2349 | CD1 | ILE B | 89 | 12.906 | 23.744 | 53.075 | 1.00 | 28.07 | C |

| | | | | | | | | | | | |
|---|---|---|---|---|---|---|---|---|---|---|---|
| ANISOU | 2349 | CD1 | ILE B | 89 | 3552 | 3886 | 3226 | −60 | 89 | 28 | C |

| | | | | | | | | | | |
|---|---|---|---|---|---|---|---|---|---|---|
| ATOM | 2350 | N | GLU B | 90 | 10.839 | 25.973 | 58.188 | 1.00 | 24.16 | N |

| | | | | | | | | | | | |
|---|---|---|---|---|---|---|---|---|---|---|---|
| ANISOU | 2350 | N | GLU B | 90 | 3025 | 3221 | 2935 | −2 | 33 | 23 | N |

| | | | | | | | | | | |
|---|---|---|---|---|---|---|---|---|---|---|
| ATOM | 2351 | CA | GLU B | 90 | 10.607 | 27.175 | 58.989 | 1.00 | 24.49 | C |

| | | | | | | | | | | | |
|---|---|---|---|---|---|---|---|---|---|---|---|
| ANISOU | 2351 | CA | GLU B | 90 | 3225 | 3207 | 2871 | −85 | 66 | 68 | C |

| | | | | | | | | | | |
|---|---|---|---|---|---|---|---|---|---|---|
| ATOM | 2352 | C | GLU B | 90 | 11.857 | 28.035 | 59.003 | 1.00 | 24.52 | C |

| | | | | | | | | | | | |
|---|---|---|---|---|---|---|---|---|---|---|---|
| ANISOU | 2352 | C | GLU B | 90 | 3213 | 3201 | 2902 | −45 | −60 | −23 | C |

| | | | | | | | | | | |
|---|---|---|---|---|---|---|---|---|---|---|
| ATOM | 2353 | O | GLU B | 90 | 12.955 | 27.534 | 59.231 | 1.00 | 27.01 | O |

| | | | | | | | | | | | |
|---|---|---|---|---|---|---|---|---|---|---|---|
| ANISOU | 2353 | O | GLU B | 90 | 3227 | 3607 | 3429 | 8 | 69 | −18 | O |

| | | | | | | | | | | |
|---|---|---|---|---|---|---|---|---|---|---|
| ATOM | 2354 | CB | GLU B | 90 | 10.329 | 26.734 | 60.450 | 1.00 | 24.61 | C |

| | | | | | | | | | | | |
|---|---|---|---|---|---|---|---|---|---|---|---|
| ANISOU | 2354 | CB | GLU B | 90 | 3184 | 3296 | 2871 | −32 | 61 | 48 | C |

| | | | | | | | | | | |
|---|---|---|---|---|---|---|---|---|---|---|
| ATOM | 2355 | CG | GLU B | 90 | 9.076 | 25.871 | 60.526 | 1.00 | 25.59 | C |

| | | | | | | | | | | | |
|---|---|---|---|---|---|---|---|---|---|---|---|
| ANISOU | 2355 | CG | GLU B | 90 | 3121 | 3430 | 3170 | −104 | −87 | −8 | C |

| | | | | | | | | | | |
|---|---|---|---|---|---|---|---|---|---|---|
| ATOM | 2356 | CD | GLU B | 90 | 8.774 | 25.403 | 61.928 | 1.00 | 25.96 | C |

| | | | | | | | | | | | |
|---|---|---|---|---|---|---|---|---|---|---|---|
| ANISOU | 2356 | CD | GLU B | 90 | 3274 | 3335 | 3253 | −120 | −62 | 21 | C |

| | | | | | | | | | | |
|---|---|---|---|---|---|---|---|---|---|---|
| ATOM | 2357 | OE1 | GLU B | 90 | 9.683 | 25.438 | 62.787 | 1.00 | 26.78 | O |

| | | | | | | | | | | | |
|---|---|---|---|---|---|---|---|---|---|---|---|
| ANISOU | 2357 | OE1 | GLU B | 90 | 3320 | 3461 | 3397 | −99 | −209 | −65 | O |

| | | | | | | | | | | |
|---|---|---|---|---|---|---|---|---|---|---|
| ATOM | 2358 | OE2 | GLU B | 90 | 7.637 | 24.958 | 62.195 | 1.00 | 25.52 | O |

| | | | | | | | | | | | |
|---|---|---|---|---|---|---|---|---|---|---|---|
| ANISOU | 2358 | OE2 | GLU B | 90 | 2889 | 3639 | 3166 | −38 | −142 | 9 | O |

| | | | | | | | | | | |
|---|---|---|---|---|---|---|---|---|---|---|
| ATOM | 2359 | N | ALA B | 91 | 11.681 | 29.330 | 58.791 | 1.00 | 24.70 | N |

| | | | | | | | | | | | |
|---|---|---|---|---|---|---|---|---|---|---|---|
| ANISOU | 2359 | N | ALA B | 91 | 3094 | 3241 | 3049 | −108 | 23 | 84 | N |

| | | | | | | | | | | |
|---|---|---|---|---|---|---|---|---|---|---|
| ATOM | 2360 | CA | ALA B | 91 | 12.813 | 30.258 | 58.866 | 1.00 | 25.40 | C |

| | | | | | | | | | | | |
|---|---|---|---|---|---|---|---|---|---|---|---|
| ANISOU | 2360 | CA | ALA B | 91 | 3151 | 3299 | 3201 | −140 | −149 | 81 | C |

| | | | | | | | | | | |
|---|---|---|---|---|---|---|---|---|---|---|
| ATOM | 2361 | C | ALA B | 91 | 12.744 | 30.993 | 60.200 | 1.00 | 26.48 | C |

| | | | | | | | | | | | |
|---|---|---|---|---|---|---|---|---|---|---|---|
| ANISOU | 2361 | C | ALA B | 91 | 3404 | 3548 | 3110 | 33 | −80 | 128 | C |

| | | | | | | | | | | |
|---|---|---|---|---|---|---|---|---|---|---|
| ATOM | 2362 | O | ALA B | 91 | 11.819 | 31.808 | 60.384 | 1.00 | 27.71 | O |

| | | | | | | | | | | | |
|---|---|---|---|---|---|---|---|---|---|---|---|
| ANISOU | 2362 | O | ALA B | 91 | 3475 | 3734 | 3321 | 126 | −220 | 56 | O |

| | | | | | | | | | | |
|---|---|---|---|---|---|---|---|---|---|---|
| ATOM | 2363 | CB | ALA B | 91 | 12.689 | 31.289 | 57.742 | 1.00 | 26.82 | C |

| | | | | | | | | | | | |
|---|---|---|---|---|---|---|---|---|---|---|---|
| ANISOU | 2363 | CB | ALA B | 91 | 3429 | 3610 | 3151 | −21 | −123 | 192 | C |

| | | | | | | | | | | |
|---|---|---|---|---|---|---|---|---|---|---|
| ATOM | 2364 | N | HIS B | 92 | 13.708 | 30.727 | 61.091 | 1.00 | 25.06 | N |

| | | | | | | | | | | | |
|---|---|---|---|---|---|---|---|---|---|---|---|
| ANISOU | 2364 | N | HIS B | 92 | 3007 | 3421 | 3094 | −168 | −69 | 96 | N |

| | | | | | | | | | | |
|---|---|---|---|---|---|---|---|---|---|---|
| ATOM | 2365 | CA | HIS B | 92 | 13.679 | 31.375 | 62.395 | 1.00 | 24.54 | C |

| | | | | | | | | | | | |
|---|---|---|---|---|---|---|---|---|---|---|---|
| ANISOU | 2365 | CA | HIS B | 92 | 3116 | 3121 | 3086 | 7 | −76 | 165 | C |

| | | | | | | | | | | |
|---|---|---|---|---|---|---|---|---|---|---|
| ATOM | 2366 | C | HIS B | 92 | 14.823 | 32.382 | 62.580 | 1.00 | 23.10 | C |

| | | | | | | | | | | | |
|---|---|---|---|---|---|---|---|---|---|---|---|
| ANISOU | 2366 | C | HIS B | 92 | 3187 | 3221 | 3130 | −61 | −92 | 160 | C |

| | | | | | | | | | | |
|---|---|---|---|---|---|---|---|---|---|---|
| ATOM | 2367 | O | HIS B | 92 | 15.915 | 32.126 | 62.119 | 1.00 | 26.43 | O |

| | | | | | | | | | | | |
|---|---|---|---|---|---|---|---|---|---|---|---|
| ANISOU | 2367 | O | HIS B | 92 | 3207 | 3463 | 3374 | −105 | −109 | 265 | O |

| | | | | | | | | | | |
|---|---|---|---|---|---|---|---|---|---|---|
| ATOM | 2368 | CB | HIS B | 92 | 13.949 | 30.292 | 63.471 | 1.00 | 24.59 | C |

| | | | | | | | | | | | |
|---|---|---|---|---|---|---|---|---|---|---|---|
| ANISOU | 2368 | CB | HIS B | 92 | 3147 | 3187 | 3007 | −35 | −22 | 205 | C |

| | | | | | | | | | | |
|---|---|---|---|---|---|---|---|---|---|---|
| ATOM | 2369 | CG | HIS B | 92 | 12.860 | 29.284 | 63.631 | 1.00 | 23.34 | C |

| | | | | | | | | | | | |
|---|---|---|---|---|---|---|---|---|---|---|---|
| ANISOU | 2369 | CG | HIS B | 92 | 2871 | 3030 | 2966 | 90 | −82 | 45 | C |

| | | | | | | | | | | |
|---|---|---|---|---|---|---|---|---|---|---|
| ATOM | 2370 | ND1 | HIS B | 92 | 11.964 | 29.387 | 64.667 | 1.00 | 23.67 | N |

| | | | | | | | | | | | |
|---|---|---|---|---|---|---|---|---|---|---|---|
| ANISOU | 2370 | ND1 | HIS B | 92 | 2897 | 3055 | 3043 | −29 | −1 | 212 | N |

| | | | | | | | | | | |
|---|---|---|---|---|---|---|---|---|---|---|
| ATOM | 2371 | CD2 | HIS H | 92 | 12.531 | 28.172 | 62.948 | 1.00 | 22.65 | C |

| | | | | | | | | | | | |
|---|---|---|---|---|---|---|---|---|---|---|---|
| ANISOU | 2371 | CD2 | HIS H | 92 | 2812 | 2915 | 2879 | 18 | −113 | 157 | C |

| | | | | | | | | | | |
|---|---|---|---|---|---|---|---|---|---|---|
| ATOM | 2372 | CE1 | HIS B | 92 | 11.097 | 28.402 | 64.606 | 1.00 | 23.25 | C |

| | | | | | | | | | | | |
|---|---|---|---|---|---|---|---|---|---|---|---|
| ANISOU | 2372 | CE1 | HIS B | 92 | 2936 | 3001 | 2894 | −43 | −101 | −47 | C |

| | | | | | | | | | | |
|---|---|---|---|---|---|---|---|---|---|---|
| ATOM | 2373 | NE2 | HIS B | 92 | 11.407 | 27.627 | 63.574 | 1.00 | 23.09 | H |

| | | | | | | | | | | | |
|---|---|---|---|---|---|---|---|---|---|---|---|
| ANISOU | 2373 | NE2 | HIS B | 92 | 2752 | 3110 | 2912 | 70 | 119 | 11 | N |

| | | | | | | | | | | |
|---|---|---|---|---|---|---|---|---|---|---|
| ATOM | 2374 | N | ARG B | 93 | 14.566 | 33.443 | 63.319 | 1.00 | 25.17 | N |

| | | | | | | | | | | | |
|---|---|---|---|---|---|---|---|---|---|---|---|
| ANISOU | 2374 | N | ARG B | 93 | 3261 | 3157 | 3147 | 16 | −168 | 226 | N |

| | | | | | | | | | | |
|---|---|---|---|---|---|---|---|---|---|---|
| ATOM | 2375 | CA | ARG B | 93 | 15.564 | 34.440 | 63.679 | 1.00 | 26.29 | C |

| | | | | | | | | | | | |
|---|---|---|---|---|---|---|---|---|---|---|---|
| ANISOU | 2375 | CA | ARG B | 93 | 3264 | 3397 | 3329 | −71 | −179 | 285 | C |

| | | | | | | | | | | |
|---|---|---|---|---|---|---|---|---|---|---|
| ATOM | 2376 | C | ARG B | 93 | 16.560 | 33.818 | 64.671 | 1.00 | 25.15 | C |

| | | | | | | | | | | | |
|---|---|---|---|---|---|---|---|---|---|---|---|
| ANISOU | 2376 | C | ARG B | 93 | 3218 | 3209 | 3128 | −100 | −80 | 232 | C |

| | | | | | | | | | | |
|---|---|---|---|---|---|---|---|---|---|---|
| ATOM | 2377 | O | ARG B | 93 | 16.153 | 33.028 | 65.553 | 1.00 | 24.62 | O |

| | | | | | | | | | | | |
|---|---|---|---|---|---|---|---|---|---|---|---|
| ANISOU | 2377 | O | ARG B | 93 | 3068 | 3166 | 3121 | −26 | −56 | 327 | O |

| | | | | | | | | | | |
|---|---|---|---|---|---|---|---|---|---|---|
| ATOM | 2378 | CB | ARG B | 93 | 14.765 | 35.572 | 64.320 | 1.00 | 30.02 | C |

| | | | | | | | | | | | |
|---|---|---|---|---|---|---|---|---|---|---|---|
| ANISOU | 2378 | CB | ARG B | 93 | 3881 | 3772 | 3754 | 103 | 38 | 150 | C |

| | | | | | | | | | | |
|---|---|---|---|---|---|---|---|---|---|---|
| ATOM | 2379 | CG | ARG B | 93 | 15.472 | 36.824 | 64.747 | 1.00 | 35.79 | C |

| | | | | | | | | | | | |
|---|---|---|---|---|---|---|---|---|---|---|---|
| ANISOU | 2379 | CG | ARG B | 93 | 4658 | 4347 | 4596 | −231 | −169 | 0 | C |

| | | | | | | | | | | |
|---|---|---|---|---|---|---|---|---|---|---|
| ATOM | 2380 | CD | ARG B | 93 | 14.488 | 37.735 | 65.519 | 1.00 | 40.21 | C |

| | | | | | | | | | | | |
|---|---|---|---|---|---|---|---|---|---|---|---|
| ANISOU | 2380 | CD | ARG B | 93 | 5143 | 5015 | 5120 | 142 | 61 | 6 | C |

| | | | | | | | | | | |
|---|---|---|---|---|---|---|---|---|---|---|
| ATOM | 2381 | NE | ARG B | 93 | 15.227 | 38.722 | 66.283 | 1.00 | 43.42 | N |

| | | | | | | | | | | | |
|---|---|---|---|---|---|---|---|---|---|---|---|
| ANISOU | 2381 | NE | ARG B | 93 | 5588 | 5389 | 5542 | −81 | −138 | −12 | N |

| | | | | | | | | | | |
|---|---|---|---|---|---|---|---|---|---|---|
| ATOM | 2382 | CZ | ARG B | 93 | 14.867 | 39.568 | 67.228 | 1.00 | 45.51 | C |

| | | | | | | | | | | | |
|---|---|---|---|---|---|---|---|---|---|---|---|
| ANISOU | 2382 | CZ | ARG B | 93 | 5870 | 5677 | 5744 | 49 | 7 | −78 | C |

| | | | | | | | | | | |
|---|---|---|---|---|---|---|---|---|---|---|
| ATOM | 2383 | NH1 | ARG B | 93 | 13.612 | 39.655 | 67.659 | 1.00 | 46.68 | N |

| | | | | | | | | | | | |
|---|---|---|---|---|---|---|---|---|---|---|---|
| ANISOU | 2383 | NH1 | ARG B | 93 | 5909 | 5935 | 5893 | −9 | 45 | 14 | N |

| | | | | | | | | | | |
|---|---|---|---|---|---|---|---|---|---|---|
| ATOM | 2384 | NH2 | ARG B | 93 | 15.802 | 40.364 | 67.762 | 1.00 | 46.46 | N |

| | | | | | | | | | | | |
|---|---|---|---|---|---|---|---|---|---|---|---|
| ANISOU | 2384 | NH2 | ARG B | 93 | 5921 | 5795 | 5937 | −29 | −27 | −69 | N |

| | | | | | | | | | | |
|---|---|---|---|---|---|---|---|---|---|---|
| ATOM | 2385 | N | GLU B | 94 | 17.835 | 34.154 | 64.532 | 1.00 | 24.37 | N |

| | | | | | | | | | | | |
|---|---|---|---|---|---|---|---|---|---|---|---|
| ANISOU | 2385 | N | GLU B | 94 | 3122 | 3107 | 3031 | 77 | −9 | 220 | N |

| | | | | | | | | | | |
|---|---|---|---|---|---|---|---|---|---|---|
| ATOM | 2386 | CA | GLU B | 94 | 18.881 | 33.732 | 65.481 | 1.00 | 23.40 | C |

| | | | | | | | | | | | |
|---|---|---|---|---|---|---|---|---|---|---|---|
| ANISOU | 2386 | CA | GLU B | 94 | 3098 | 2916 | 2878 | 4 | 20 | 217 | C |

| | | | | | | | | | | |
|---|---|---|---|---|---|---|---|---|---|---|
| ATOM | 2387 | C | GLU B | 94 | 18.391 | 34.161 | 66.860 | 1.00 | 21.74 | C |

| | | | | | | | | | | | |
|---|---|---|---|---|---|---|---|---|---|---|---|
| ANISOU | 2387 | C | GLU B | 94 | 2883 | 2678 | 2697 | −79 | −97 | 290 | C |

| | | | | | | | | | | |
|---|---|---|---|---|---|---|---|---|---|---|
| ATOM | 2388 | O | GLU B | 94 | 17.644 | 35.152 | 67.020 | 1.00 | 21.06 | O |

| | | | | | | | | | | | |
|---|---|---|---|---|---|---|---|---|---|---|---|
| ANISOU | 2388 | O | GLU B | 94 | 3010 | 2628 | 2365 | −63 | −18 | 350 | O |

| | | | | | | | | | | |
|---|---|---|---|---|---|---|---|---|---|---|
| ATOM | 2389 | CB | GLU B | 94 | 20.226 | 34.432 | 65.247 | 1.00 | 27.46 | C |

| | | | | | | | | | | | |
|---|---|---|---|---|---|---|---|---|---|---|---|
| ANISOU | 2389 | CB | GLU B | 94 | 3263 | 3537 | 3633 | −91 | 195 | 207 | C |

| | | | | | | | | | | |
|---|---|---|---|---|---|---|---|---|---|---|
| ATOM | 2390 | CG | GLU B | 94 | 21.258 | 34.449 | 66.351 | 1.00 | 32.08 | C |

| | | | | | | | | | | | |
|---|---|---|---|---|---|---|---|---|---|---|---|
| ANISOU | 2390 | CG | GLU B | 94 | 4019 | 4147 | 4023 | −45 | −150 | 167 | C |

| | | | | | | | | | | |
|---|---|---|---|---|---|---|---|---|---|---|
| ATOM | 2391 | CD | GLU B | 94 | 22.579 | 35.128 | 66.043 | 1.00 | 35.29 | C |

| | | | | | | | | | | | |
|---|---|---|---|---|---|---|---|---|---|---|---|
| ANISOU | 2391 | CD | GLU B | 94 | 4167 | 4527 | 4715 | −120 | 74 | 160 | C |

| | | | | | | | | | | |
|---|---|---|---|---|---|---|---|---|---|---|
| ATOM | 2392 | OE1 | GLU B | 94 | 22.562 | 36.224 | 65.424 | 1.00 | 36.54 | O |

| | | | | | | | | | | | |
|---|---|---|---|---|---|---|---|---|---|---|---|
| ANISOU | 2392 | OE1 | GLU B | 94 | 4394 | 4616 | 4872 | −37 | 125 | 272 | O |

| | | | | | | | | | | |
|---|---|---|---|---|---|---|---|---|---|---|
| ATOM | 2393 | OE2 | GLU B | 94 | 23.695 | 34.650 | 66.419 | 1.00 | 36.86 | O |

| | | | | | | | | | | | |
|---|---|---|---|---|---|---|---|---|---|---|---|
| ANISOU | 2393 | OE2 | GLU B | 94 | 4582 | 4548 | 4876 | 95 | 21 | 165 | O |

| | | | | | | | | | | |
|---|---|---|---|---|---|---|---|---|---|---|
| ATOM | 2394 | N | GLN B | 95 | 18.801 | 33.366 | 67.875 | 1.00 | 19.64 | N |

| | | | | | | | | | | | |
|---|---|---|---|---|---|---|---|---|---|---|---|
| ANISOU | 2394 | N | GLN B | 95 | 2618 | 2349 | 2494 | −82 | 46 | 174 | N |

| | | | | | | | | | | |
|---|---|---|---|---|---|---|---|---|---|---|
| ATOM | 2395 | CA | GLN B | 95 | 18.424 | 33.720 | 69.241 | 1.00 | 18.66 | C |

| | | | | | | | | | | | |
|---|---|---|---|---|---|---|---|---|---|---|---|
| ANISOU | 2395 | CA | GLN B | 95 | 2531 | 2133 | 2427 | −190 | −26 | 154 | C |

| | | | | | | | | | | |
|---|---|---|---|---|---|---|---|---|---|---|
| ATOM | 2396 | C | GLN B | 95 | 19.717 | 33.972 | 70.027 | 1.00 | 17.79 | C |

| | | | | | | | | | | | |
|---|---|---|---|---|---|---|---|---|---|---|---|
| ANISOU | 2396 | C | GLN B | 95 | 2290 | 2271 | 2199 | −4 | 17 | 200 | C |

| | | | | | | | | | | |
|---|---|---|---|---|---|---|---|---|---|---|
| ATOM | 2397 | O | GLN B | 95 | 20.686 | 33.195 | 69.891 | 1.00 | 19.08 | O |

| | | | | | | | | | | | |
|---|---|---|---|---|---|---|---|---|---|---|---|
| ANISOU | 2397 | O | GLN B | 95 | 2482 | 2421 | 2346 | 31 | −243 | 66 | O |

| | | | | | | | | | | |
|---|---|---|---|---|---|---|---|---|---|---|
| ATOM | 2398 | CB | GLN B | 95 | 17.664 | 32.611 | 70.007 | 1.00 | 17.94 | C |

| | | | | | | | | | | | |
|---|---|---|---|---|---|---|---|---|---|---|---|
| ANISOU | 2398 | CB | GLN B | 95 | 2313 | 2129 | 2376 | −120 | −69 | 166 | C |

| | | | | | | | | | | |
|---|---|---|---|---|---|---|---|---|---|---|
| ATOM | 2399 | CG | GLN B | 95 | 16.305 | 32.372 | 69.311 | 1.00 | 18.66 | C |

| | | | | | | | | | | | |
|---|---|---|---|---|---|---|---|---|---|---|---|
| ANISOU | 2399 | CG | GLN B | 95 | 2334 | 2316 | 2438 | −32 | −176 | 240 | C |

| | | | | | | | | | | |
|---|---|---|---|---|---|---|---|---|---|---|
| ATOM | 2400 | CD | GLN B | 95 | 15.559 | 31.171 | 69.883 | 1.00 | 20.08 | C |

| | | | | | | | | | | | |
|---|---|---|---|---|---|---|---|---|---|---|---|
| ANISOU | 2400 | CD | GLN B | 95 | 2645 | 2434 | 2552 | −216 | −34 | 7 | C |

| | | | | | | | | | | |
|---|---|---|---|---|---|---|---|---|---|---|
| ATOM | 2401 | OE1 | GLN B | 95 | 16.210 | 30.391 | 70.569 | 1.00 | 21.94 | O |

| | | | | | | | | | | | |
|---|---|---|---|---|---|---|---|---|---|---|---|
| ANISOU | 2401 | OE1 | GLN B | 95 | 2934 | 2518 | 2883 | −71 | 1 | 136 | O |

| | | | | | | | | | | |
|---|---|---|---|---|---|---|---|---|---|---|
| ATOM | 2402 | NE2 | GLN B | 95 | 14.280 | 31.092 | 69.613 | 1.00 | 20.24 | N |

| | | | | | | | | | | | |
|---|---|---|---|---|---|---|---|---|---|---|---|
| ANISOU | 2402 | NE2 | GLN B | 95 | 2640 | 2405 | 2647 | 77 | −81 | 173 | N |

| | | | | | | | | | | |
|---|---|---|---|---|---|---|---|---|---|---|
| ATOM | 2403 | N | ILE B | 96 | 19.701 | 35.005 | 70.871 | 1.00 | 19.29 | N |

| | | | | | | | | | | | |
|---|---|---|---|---|---|---|---|---|---|---|---|
| ANISOU | 2403 | N | ILE B | 96 | 2494 | 2188 | 2646 | −118 | −79 | 139 | N |

| | | | | | | | | | | |
|---|---|---|---|---|---|---|---|---|---|---|
| ATOM | 2404 | CA | ILE B | 96 | 20.823 | 35.279 | 71.764 | 1.00 | 19.53 | C |

| | | | | | | | | | | | |
|---|---|---|---|---|---|---|---|---|---|---|---|
| ANISOU | 2404 | CA | ILE B | 96 | 2611 | 2384 | 2424 | −68 | 5 | 85 | C |

| | | | | | | | | | | |
|---|---|---|---|---|---|---|---|---|---|---|
| ATOM | 2405 | C | ILE B | 96 | 20.254 | 35.486 | 73.178 | 1.00 | 18.46 | C |

| | | | | | | | | | | | |
|---|---|---|---|---|---|---|---|---|---|---|---|
| ANISOU | 2405 | C | ILE B | 96 | 2689 | 1956 | 2370 | 16 | 23 | 129 | C |

| | | | | | | | | | | |
|---|---|---|---|---|---|---|---|---|---|---|
| ATOM | 2406 | O | ILE B | 96 | 19.038 | 35.698 | 73.361 | 1.00 | 17.67 | O |

| | | | | | | | | | | | |
|---|---|---|---|---|---|---|---|---|---|---|---|
| ANISOU | 2406 | O | ILE B | 96 | 2559 | 2087 | 2067 | 37 | 57 | 79 | O |

| | | | | | | | | | | |
|---|---|---|---|---|---|---|---|---|---|---|
| ATOM | 2407 | CB | ILE B | 96 | 21.644 | 36.547 | 71.412 | 1.00 | 21.68 | C |

| | | | | | | | | | | | |
|---|---|---|---|---|---|---|---|---|---|---|---|
| ANISOU | 2407 | CB | ILE B | 96 | 2942 | 2501 | 2793 | −144 | 82 | 117 | C |

| | | | | | | | | | | |
|---|---|---|---|---|---|---|---|---|---|---|
| ATOM | 2408 | CG1 | ILE B | 96 | 20.722 | 37.767 | 71.378 | 1.00 | 22.68 | C |

| | | | | | | | | | | | |
|---|---|---|---|---|---|---|---|---|---|---|---|
| ANISOU | 2408 | CG1 | ILE B | 96 | 3119 | 2624 | 2873 | −66 | 13 | 167 | C |

| | | | | | | | | | | |
|---|---|---|---|---|---|---|---|---|---|---|
| ATOM | 2409 | CG2 | ILE B | 96 | 22.299 | 36.340 | 70.046 | 1.00 | 24.38 | C |

| | | | | | | | | | | | |
|---|---|---|---|---|---|---|---|---|---|---|---|
| ANISOU | 2409 | CG2 | ILE B | 96 | 3177 | 3023 | 3063 | −232 | 295 | 23 | C |

| | | | | | | | | | | |
|---|---|---|---|---|---|---|---|---|---|---|
| ATOM | 2410 | CD1 | ILE B | 96 | 21.459 | 39.090 | 71.209 | 1.00 | 24.56 | C |

| | | | | | | | | | | | |
|---|---|---|---|---|---|---|---|---|---|---|---|
| ANISOU | 2410 | CD1 | ILE B | 96 | 3550 | 2749 | 3034 | −222 | 56 | 68 | C |

| | | | | | | | | | | |
|---|---|---|---|---|---|---|---|---|---|---|
| ATOM | 2411 | N | GLY B | 97 | 21.127 | 35.430 | 74.181 | 1.00 | 17.64 | N |

| | | | | | | | | | | | |
|---|---|---|---|---|---|---|---|---|---|---|---|
| ANISOU | 2411 | N | GLY B | 97 | 2402 | 1926 | 2374 | −78 | 97 | 56 | N |

| | | | | | | | | | | |
|---|---|---|---|---|---|---|---|---|---|---|
| ATOM | 2412 | CA | GLY B | 97 | 20.667 | 35.692 | 75.527 | 1.00 | 16.66 | C |

| | | | | | | | | | | | |
|---|---|---|---|---|---|---|---|---|---|---|---|
| ANISOU | 2412 | CA | GLY B | 97 | 2257 | 1869 | 2203 | 40 | −122 | 173 | C |

| | | | | | | | | | | |
|---|---|---|---|---|---|---|---|---|---|---|
| ATOM | 2413 | C | GLY B | 97 | 21.833 | 35.730 | 76.508 | 1.00 | 16.37 | C |

| | | | | | | | | | | | |
|---|---|---|---|---|---|---|---|---|---|---|---|
| ANISOU | 2413 | C | GLY B | 97 | 2346 | 1846 | 2027 | −48 | 15 | 65 | C |

| | | | | | | | | | | |
|---|---|---|---|---|---|---|---|---|---|---|
| ATOM | 2414 | O | GLY B | 97 | 22.844 | 35.095 | 76.262 | 1.00 | 15.60 | O |

| | | | | | | | | | | | |
|---|---|---|---|---|---|---|---|---|---|---|---|
| ANISOU | 2414 | 0 | GLY B | 97 | 2294 | 1889 | 1744 | 22 | 100 | 33 | O |

| | | | | | | | | | | |
|---|---|---|---|---|---|---|---|---|---|---|
| ATOM | 2415 | N | GLY B | 98 | 21.589 | 36.477 | 77.568 | 1.00 | 15.68 | N |

| | | | | | | | | | | | |
|---|---|---|---|---|---|---|---|---|---|---|---|
| ANISOU | 2415 | N | GLY B | 98 | 2453 | 1617 | 1888 | −159 | 135 | 180 | N |

| | | | | | | | | | | |
|---|---|---|---|---|---|---|---|---|---|---|
| ATOM | 2416 | CA | GLY B | 98 | 22.595 | 36.730 | 78.598 | 1.00 | 16.42 | C |

| | | | | | | | | | | | |
|---|---|---|---|---|---|---|---|---|---|---|---|
| ANISOU | 2416 | CA | GLY B | 98 | 2469 | 1719 | 2050 | −46 | 21 | 75 | C |

| | | | | | | | | | | |
|---|---|---|---|---|---|---|---|---|---|---|
| ATOM | 2417 | C | GLY B | 98 | 22.176 | 35.984 | 79.863 | 1.00 | 16.46 | C |

| | | | | | | | | | | | |
|---|---|---|---|---|---|---|---|---|---|---|---|
| ANISOU | 2417 | C | GLY B | 98 | 2600 | 1615 | 2039 | −96 | −27 | −56 | C |

| | | | | | | | | | | |
|---|---|---|---|---|---|---|---|---|---|---|
| ATOM | 2418 | O | GLY B | 98 | 23.034 | 36.071 | 80.909 | 1.00 | 17.02 | O |

| | | | | | | | | | | | |
|---|---|---|---|---|---|---|---|---|---|---|---|
| ANISOU | 2418 | O | GLY B | 98 | 2479 | 1915 | 2071 | 132 | −111 | 62 | O |
| TER | 2419 | | GLY B | 98 | | | | | | | |

| | | | | | | | | | | |
|---|---|---|---|---|---|---|---|---|---|---|
| HETATM | 2420 | O | HOH | 1 | 19.651 | 30.058 | 83.232 | 1.00 | 17.25 | O |

| | | | | | | | | | | | |
|---|---|---|---|---|---|---|---|---|---|---|---|
| ANISOU | 2420 | O | HOH | 1 | 2472 | 1924 | 2157 | −142 | 353 | −122 | O |

| | | | | | | | | | | |
|---|---|---|---|---|---|---|---|---|---|---|
| HETATM | 2421 | O | HOH | 2 | 19.243 | 26.284 | 69.954 | 1.00 | 19.68 | O |

| | | | | | | | | | | | |
|---|---|---|---|---|---|---|---|---|---|---|---|
| ANISOU | 2421 | O | HOH | 2 | 2524 | 2156 | 2796 | −140 | 74 | 198 | O |

| | | | | | | | | | | |
|---|---|---|---|---|---|---|---|---|---|---|
| HETATM | 2422 | O | HOH | 3 | 24.806 | 33.659 | 77.583 | 1.00 | 16.28 | O |

| | | | | | | | | | | | |
|---|---|---|---|---|---|---|---|---|---|---|---|
| ANIBON | 2422 | O | HOH | 3 | 2295 | 1881 | 2009 | −78 | 238 | 120 | O |

| | | | | | | | | | | |
|---|---|---|---|---|---|---|---|---|---|---|
| BETATH | 2423 | O | HOH | 4 | 35.539 | 13.854 | 89.780 | 1.00 | 27.42 | O |

| | | | | | | | | | | | |
|---|---|---|---|---|---|---|---|---|---|---|---|
| ANISOU | 2423 | O | HOH | 4 | 3329 | 2782 | 4306 | −453 | −173 | −252 | O |

| | | | | | | | | | | |
|---|---|---|---|---|---|---|---|---|---|---|
| HETATM | 2424 | O | HOH | 5 | 13.626 | 24.057 | 73.516 | 1.00 | 16.34 | O |

| | | | | | | | | | | | |
|---|---|---|---|---|---|---|---|---|---|---|---|
| ANISOU | 2424 | O | HOH | 5 | 2197 | 1908 | 2103 | −56 | 25 | 16 | O |

| | | | | | | | | | | |
|---|---|---|---|---|---|---|---|---|---|---|
| HETATM | 2425 | O | HOH | 6 | 19.889 | 20.307 | 63.946 | 1.00 | 24.27 | O |

| | | | | | | | | | | | |
|---|---|---|---|---|---|---|---|---|---|---|---|
| ANISOU | 2425 | O | HOH | 6 | 3109 | 3221 | 2889 | 93 | −240 | 84 | O |

| | | | | | | | | | | |
|---|---|---|---|---|---|---|---|---|---|---|
| HETATM | 2426 | O | HOH | 7 | 19.459 | 27.209 | 83.447 | 1.00 | 19.36 | O |

| | | | | | | | | | | | |
|---|---|---|---|---|---|---|---|---|---|---|---|
| ANISOU | 2426 | O | HOH | 7 | 3087 | 2146 | 2122 | −11 | 454 | 219 | O |

| | | | | | | | | | | |
|---|---|---|---|---|---|---|---|---|---|---|
| HETATM | 2427 | O | HOH | 8 | 36.110 | 32.059 | 76.728 | 1.00 | 20.80 | O |

| | | | | | | | | | | | |
|---|---|---|---|---|---|---|---|---|---|---|---|
| ANISOU | 2427 | O | HOH | 8 | 2587 | 2271 | 3045 | −111 | −177 | 46 | O |

| | | | | | | | | | | |
|---|---|---|---|---|---|---|---|---|---|---|
| HETATM | 2428 | O | HOH | 9 | 39.603 | 34.562 | 74.994 | 1.00 | 25.42 | O |

| | | | | | | | | | | | |
|---|---|---|---|---|---|---|---|---|---|---|---|
| ANISOU | 2428 | O | HOH | 9 | 3037 | 2989 | 3634 | −155 | 271 | −117 | O |

| | | | | | | | | | | |
|---|---|---|---|---|---|---|---|---|---|---|
| HETATM | 2429 | O | HOH | 10 | 7.319 | 15.790 | 68.932 | 1.00 | 23.05 | O |

| | | | | | | | | | | | |
|---|---|---|---|---|---|---|---|---|---|---|---|
| ANISOU | 2429 | O | HOH | 10 | 2891 | 3023 | 2845 | −235 | 121 | −301 | O |

| | | | | | | | | | | |
|---|---|---|---|---|---|---|---|---|---|---|
| HETATM | 2430 | O | HOH | 11 | 36.269 | 31.500 | 79.570 | 1.00 | 24.80 | O |

| | | | | | | | | | | | |
|---|---|---|---|---|---|---|---|---|---|---|---|
| ANISOU | 2430 | O | HOH | 11 | 3213 | 3256 | 2953 | −161 | −343 | −87 | O |

| | | | | | | | | | | |
|---|---|---|---|---|---|---|---|---|---|---|
| HETATM | 2431 | O | HOH | 12 | 28.941 | 41.240 | 78.512 | 1.00 | 23.16 | O |

| | | | | | | | | | | | |
|---|---|---|---|---|---|---|---|---|---|---|---|
| ANISOU | 2431 | O | HOH | 12 | 3083 | 2195 | 3523 | −76 | 314 | 91 | O |

| | | | | | | | | | | |
|---|---|---|---|---|---|---|---|---|---|---|
| HETATM | 2432 | O | HOH | 13 | 13.566 | 17.047 | 65.780 | 1.00 | 26.41 | O |

| | | | | | | | | | | | |
|---|---|---|---|---|---|---|---|---|---|---|---|
| ANISOU | 2432 | O | HOH | 13 | 3433 | 3556 | 3045 | −340 | −104 | −348 | O |

| | | | | | | | | | | |
|---|---|---|---|---|---|---|---|---|---|---|
| HETATM | 2433 | O | HOH | 14 | 26.963 | 30.646 | 90.501 | 1.00 | 22.12 | O |

| | | | | | | | | | | | |
|---|---|---|---|---|---|---|---|---|---|---|---|
| ANISOU | 2433 | O | HOH | 14 | 3325 | 2856 | 2223 | −319 | −245 | −104 | O |

| | | | | | | | | | | |
|---|---|---|---|---|---|---|---|---|---|---|
| HETATM | 2434 | O | HOH | 15 | 37.785 | 24.360 | 80.585 | 1.00 | 24.25 | O |

| | | | | | | | | | | | |
|---|---|---|---|---|---|---|---|---|---|---|---|
| ANISOU | 2434 | O | HOH | 15 | 2761 | 3370 | 3083 | 482 | −101 | 86 | O |

| | | | | | | | | | | |
|---|---|---|---|---|---|---|---|---|---|---|
| HETATM | 2435 | O | HOH | 16 | 19.263 | 19.400 | 67.772 | 1.00 | 30.56 | O |

| | | | | | | | | | | | |
|---|---|---|---|---|---|---|---|---|---|---|---|
| ANISOU | 2435 | O | HOH | 16 | 3589 | 3688 | 4336 | −126 | −231 | 86 | O |

| | | | | | | | | | | |
|---|---|---|---|---|---|---|---|---|---|---|
| HETATM | 2436 | O | HOH | 17 | 33.444 | 35.183 | 80.828 | 1.00 | 22.86 | O |

| | | | | | | | | | | | |
|---|---|---|---|---|---|---|---|---|---|---|---|
| ANISOU | 2436 | O | HOH | 17 | 3448 | 2497 | 2742 | 316 | −292 | 219 | O |

| | | | | | | | | | | |
|---|---|---|---|---|---|---|---|---|---|---|
| HETATM | 2437 | O | HOH | 18 | 19.956 | 21.117 | 86.302 | 1.00 | 23.06 | O |

| | | | | | | | | | | | |
|---|---|---|---|---|---|---|---|---|---|---|---|
| ANISOU | 2437 | O | HOH | 18 | 3394 | 2177 | 3190 | 43 | 28 | 175 | O |

| | | | | | | | | | | |
|---|---|---|---|---|---|---|---|---|---|---|
| HETATM | 2438 | O | HOH | 19 | 12.619 | 17.491 | 86.136 | 1.00 | 29.62 | O |

| | | | | | | | | | | | |
|---|---|---|---|---|---|---|---|---|---|---|---|
| ANISOU | 2438 | O | HOH | 19 | 3541 | 3718 | 3996 | −50 | 55 | −99 | O |

| | | | | | | | | | | |
|---|---|---|---|---|---|---|---|---|---|---|
| HETATM | 2439 | O | HOH | 20 | 13.054 | 28.919 | 73.370 | 1.00 | 20.76 | O |

| | | | | | | | | | | | |
|---|---|---|---|---|---|---|---|---|---|---|---|
| ANISOU | 2439 | O | HOH | 20 | 2517 | 2230 | 3140 | 18 | −17 | −64 | O |

| | | | | | | | | | | |
|---|---|---|---|---|---|---|---|---|---|---|
| HETATM | 2440 | O | HOH | 21 | 35.696 | 38.599 | 74.730 | 1.00 | 22.85 | O |

| | | | | | | | | | | | |
|---|---|---|---|---|---|---|---|---|---|---|---|
| ANISOU | 2440 | O | HOH | 21 | 3062 | 2488 | 3132 | −290 | 295 | −364 | O |

| | | | | | | | | | | |
|---|---|---|---|---|---|---|---|---|---|---|
| HETATM | 2441 | O | HOH | 22 | 24.135 | 9.921 | 89.518 | 1.00 | 30.44 | O |

| | | | | | | | | | | | |
|---|---|---|---|---|---|---|---|---|---|---|---|
| ANISOU | 2441 | O | HOH | 22 | 3960 | 3382 | 4226 | 285 | 12 | −53 | O |

| | | | | | | | | | | |
|---|---|---|---|---|---|---|---|---|---|---|
| HETATM | 2442 | O | HOH | 23 | 2.463 | 9.690 | 84.033 | 1.00 | 28.47 | O |

| | | | | | | | | | | | |
|---|---|---|---|---|---|---|---|---|---|---|---|
| ANISOU | 2442 | O | HOH | 23 | 3535 | 3243 | 4038 | −675 | −51 | 93 | O |

| | | | | | | | | | | |
|---|---|---|---|---|---|---|---|---|---|---|
| HETATM | 2443 | O | HOH | 24 | 31.799 | 39.656 | 85.633 | 1.00 | 23.50 | O |

| | | | | | | | | | | | |
|---|---|---|---|---|---|---|---|---|---|---|---|
| ANISOU | 2443 | O | HOH | 24 | 3441 | 2251 | 3238 | −157 | −71 | −8 | O |

| | | | | | | | | | | |
|---|---|---|---|---|---|---|---|---|---|---|
| HETATM | 2444 | O | HOH | 25 | 32.112 | 33.143 | 32.096 | 1.00 | 23.76 | O |

| | | | | | | | | | | | |
|---|---|---|---|---|---|---|---|---|---|---|---|
| ANISOU | 2444 | O | HOH | 25 | 3332 | 2950 | 2745 | 595 | 247 | 387 | O |

| | | | | | | | | | | |
|---|---|---|---|---|---|---|---|---|---|---|
| HETATM | 2445 | O | HOH | 26 | 7.693 | 22.413 | 63.301 | 1.00 | 29.01 | O |

| | | | | | | | | | | | |
|---|---|---|---|---|---|---|---|---|---|---|---|
| ANISOU | 2445 | O | HOH | 26 | 3742 | 3973 | 3307 | 63 | −5 | 279 | O |

| | | | | | | | | | | |
|---|---|---|---|---|---|---|---|---|---|---|
| HETATM | 2446 | O | HOH | 27 | 14.534 | 34.761 | 35.038 | 1.00 | 26.28 | O |

| | | | | | | | | | | | |
|---|---|---|---|---|---|---|---|---|---|---|---|
| ANISOU | 2446 | O | HOH | 27 | 3462 | 2768 | 3757 | 473 | 100 | −20 | O |

| | | | | | | | | | | |
|---|---|---|---|---|---|---|---|---|---|---|
| HETATM | 2447 | O | HOH | 28 | 11.023 | 22.853 | 61.991 | 1.00 | 25.33 | O |

| | | | | | | | | | | | |
|---|---|---|---|---|---|---|---|---|---|---|---|
| ANISOU | 2447 | O | HOH | 28 | 3027 | 3163 | 3434 | −78 | 96 | 41 | O |

| | | | | | | | | | | |
|---|---|---|---|---|---|---|---|---|---|---|
| HETATM | 2448 | O | HOH | 29 | 22.313 | 30.321 | 59.892 | 1.00 | 39.42 | O |

| | | | | | | | | | | | |
|---|---|---|---|---|---|---|---|---|---|---|---|
| ANISOU | 2449 | O | HOH | 29 | 5046 | 5136 | 4797 | −61 | 77 | −36 | O |

| | | | | | | | | | | |
|---|---|---|---|---|---|---|---|---|---|---|
| HETATM | 2449 | O | HOH | 30 | 31.490 | 17.263 | 66.262 | 1.00 | 24.59 | O |

| | | | | | | | | | | | |
|---|---|---|---|---|---|---|---|---|---|---|---|
| ANISOU | 2449 | O | HOH | 30 | 2982 | 3006 | 3355 | −67 | 237 | −316 | O |

| | | | | | | | | | | |
|---|---|---|---|---|---|---|---|---|---|---|
| HETATM | 2450 | O | HOH | 31 | 27.364 | 41.768 | 84.190 | 1.00 | 26.32 | O |

| | | | | | | | | | | | |
|---|---|---|---|---|---|---|---|---|---|---|---|
| ANISOU | 2450 | O | HOH | 31 | 3256 | 2898 | 3847 | −75 | 105 | 106 | O |

| | | | | | | | | | | |
|---|---|---|---|---|---|---|---|---|---|---|
| HETATM | 2451 | O | HOH | 32 | 12.813 | 33.953 | 71.853 | 1.00 | 27.15 | O |

| | | | | | | | | | | | |
|---|---|---|---|---|---|---|---|---|---|---|---|
| ANISOU | 2451 | O | HOH | 32 | 3106 | 3587 | 3623 | 23 | −590 | −124 | O |

| | | | | | | | | | | |
|---|---|---|---|---|---|---|---|---|---|---|
| HETATM | 2452 | O | HOH | 33 | 30.540 | 20.936 | 90.768 | 1.00 | 29.41 | O |

| | | | | | | | | | | | |
|---|---|---|---|---|---|---|---|---|---|---|---|
| ANISOU | 2452 | O | HOH | 33 | 3725 | 4009 | 3443 | −67 | −314 | 263 | O |

| | | | | | | | | | | |
|---|---|---|---|---|---|---|---|---|---|---|
| HETATM | 2453 | O | HOH | 34 | 29.469 | 9.066 | 77.394 | 1.00 | 31.55 | O |

| | | | | | | | | | | | |
|---|---|---|---|---|---|---|---|---|---|---|---|
| ANISOU | 2453 | O | HOH | 34 | 4090 | 3174 | 4725 | −10 | −76 | 67 | O |

| | | | | | | | | | | |
|---|---|---|---|---|---|---|---|---|---|---|
| HETATM | 2454 | O | HOH | 35 | 7.046 | 9.814 | 70.879 | 1.00 | 27.40 | O |

| | | | | | | | | | | | |
|---|---|---|---|---|---|---|---|---|---|---|---|
| ANISOU | 2454 | O | HOH | 35 | 3554 | 3351 | 3507 | −30 | −102 | −126 | O |

| | | | | | | | | | | |
|---|---|---|---|---|---|---|---|---|---|---|
| HETATM | 2455 | O | HOH | 36 | 11.781 | 34.052 | 64.099 | 1.00 | 26.97 | O |

| | | | | | | | | | | | |
|---|---|---|---|---|---|---|---|---|---|---|---|
| ANISOU | 2455 | O | HOH | 36 | 3606 | 3451 | 3191 | −7 | −188 | 104 | O |

| | | | | | | | | | | |
|---|---|---|---|---|---|---|---|---|---|---|
| HETATM | 2456 | O | HOH | 37 | 13.747 | 32.917 | 67.074 | 1.00 | 25.84 | O |

| | | | | | | | | | | | |
|---|---|---|---|---|---|---|---|---|---|---|---|
| ANISOU | 2456 | O | HOH | 37 | 3150 | 3070 | 3599 | 138 | −234 | 29 | O |

| | | | | | | | | | | |
|---|---|---|---|---|---|---|---|---|---|---|
| HETATM | 2457 | O | HOH | 38 | 40.989 | 22.800 | 71.958 | 1.00 | 30.02 | O |

| | | | | | | | | | | | |
|---|---|---|---|---|---|---|---|---|---|---|---|
| ANISOU | 2457 | O | HOH | 38 | 3543 | 3952 | 3909 | 370 | 253 | −161 | O |

| | | | | | | | | | | |
|---|---|---|---|---|---|---|---|---|---|---|
| HETATM | 2458 | O | HOH | 39 | 35.724 | 20.786 | 64.481 | 1.00 | 31.70 | O |

| | | | | | | | | | | | |
|---|---|---|---|---|---|---|---|---|---|---|---|
| ANISOU | 2458 | O | HOH | 39 | 3747 | 4130 | 4169 | 41 | 54 | −302 | O |

| | | | | | | | | | | |
|---|---|---|---|---|---|---|---|---|---|---|
| HETATM | 2459 | O | HOH | 40 | 29.248 | 31.177 | 92.034 | 1.00 | 27.04 | O |

| | | | | | | | | | | | |
|---|---|---|---|---|---|---|---|---|---|---|---|
| ANISOU | 2459 | O | HOH | 40 | 3561 | 3525 | 3189 | 41 | 106 | −52 | O |

| | | | | | | | | | | |
|---|---|---|---|---|---|---|---|---|---|---|
| HETATM | 2460 | O | HOH | 41 | 16.731 | 19.308 | 68.657 | 1.00 | 23.24 | O |

| | | | | | | | | | | | |
|---|---|---|---|---|---|---|---|---|---|---|---|
| ANISOU | 2460 | O | HOH | 41 | 2932 | 2937 | 2962 | −35 | 18 | −260 | O |

| | | | | | | | | | | |
|---|---|---|---|---|---|---|---|---|---|---|
| HETATM | 2461 | O | HOH | 42 | 11.592 | 31.817 | 65.888 | 1.00 | 29.99 | O |

| | | | | | | | | | | | |
|---|---|---|---|---|---|---|---|---|---|---|---|
| ANISOU | 2461 | O | HOH | 42 | 3548 | 4075 | 3733 | 54 | 315 | 103 | O |

| | | | | | | | | | | |
|---|---|---|---|---|---|---|---|---|---|---|
| HETATM | 2462 | O | HOH | 43 | 9.517 | 22.269 | 59.558 | 1.00 | 24.82 | O |

| | | | | | | | | | | | |
|---|---|---|---|---|---|---|---|---|---|---|---|
| ANISOU | 2462 | O | HOH | 43 | 3149 | 3622 | 2660 | 111 | −171 | −88 | O |

| | | | | | | | | | | |
|---|---|---|---|---|---|---|---|---|---|---|
| HETATM | 2463 | O | HOH | 44 | 15.935 | 10.353 | 84.557 | 1.00 | 30.61 | O |

| | | | | | | | | | | | |
|---|---|---|---|---|---|---|---|---|---|---|---|
| ANISOU | 2463 | O | HOH | 44 | 3701 | 3624 | 4304 | −88 | −58 | 18 | O |

| | | | | | | | | | | |
|---|---|---|---|---|---|---|---|---|---|---|
| HETATM | 2464 | O | HOH | 45 | 28.604 | 41.191 | 88.587 | 1.00 | 28.52 | O |

| | | | | | | | | | | | |
|---|---|---|---|---|---|---|---|---|---|---|---|
| ANISOU | 2464 | O | HOH | 45 | 3662 | 3098 | 4075 | −223 | 172 | 91 | O |

| | | | | | | | | | | |
|---|---|---|---|---|---|---|---|---|---|---|
| HETATM | 2465 | O | HOH | 46 | 35.793 | 33.851 | 80.461 | 1.00 | 29.90 | O |

| | | | | | | | | | | | |
|---|---|---|---|---|---|---|---|---|---|---|---|
| ANISOU | 2465 | O | HOH | 46 | 3664 | 3710 | 3987 | 59 | −40 | 237 | O |

| | | | | | | | | | | |
|---|---|---|---|---|---|---|---|---|---|---|
| HETATM | 2466 | O | HOH | 47 | 40.058 | 25.649 | 81.260 | 1.00 | 33.89 | O |

| | | | | | | | | | | | |
|---|---|---|---|---|---|---|---|---|---|---|---|
| ANISOU | 2466 | O | HOH | 47 | 3757 | 4632 | 4485 | −46 | −155 | 80 | O |

| | | | | | | | | | | |
|---|---|---|---|---|---|---|---|---|---|---|
| HETATM | 2467 | O | HOH | 48 | 17.335 | 25.487 | 46.362 | 1.00 | 36.44 | O |

| | | | | | | | | | | | |
|---|---|---|---|---|---|---|---|---|---|---|---|
| ANISOU | 2467 | O | HOH | 48 | 4492 | 4989 | 4366 | 92 | 344 | 122 | O |

| | | | | | | | | | | |
|---|---|---|---|---|---|---|---|---|---|---|
| HETATM | 2468 | O | HOH | 49 | 35.363 | 39.486 | 72.192 | 1.00 | 33.90 | O |

| | | | | | | | | | | | |
|---|---|---|---|---|---|---|---|---|---|---|---|
| ANISOU | 2468 | O | HOH | 49 | 4422 | 4305 | 4152 | −46 | −124 | −128 | O |

| | | | | | | | | | | |
|---|---|---|---|---|---|---|---|---|---|---|
| HETATM | 2469 | O | HOH | 50 | 31.905 | 33.410 | 85.105 | 1.00 | 28.47 | O |

| | | | | | | | | | | | |
|---|---|---|---|---|---|---|---|---|---|---|---|
| ANISOU | 2469 | O | HOH | 50 | 4198 | 2777 | 3842 | 162 | −364 | −37 | O |

| | | | | | | | | | | |
|---|---|---|---|---|---|---|---|---|---|---|
| HETATM | 2470 | O | HOH | 51 | 20.232 | 8.433 | 81.394 | 1.00 | 37.25 | O |

| | | | | | | | | | | | |
|---|---|---|---|---|---|---|---|---|---|---|---|
| ANISOU | 2470 | O | HOH | 51 | 4842 | 4543 | 4767 | 23 | 177 | −76 | O |

| | | | | | | | | | | |
|---|---|---|---|---|---|---|---|---|---|---|
| HETATM | 2471 | O | HOH | 52 | 23.343 | 22.748 | 60.568 | 1.00 | 30.09 | O |

| | | | | | | | | | | | |
|---|---|---|---|---|---|---|---|---|---|---|---|
| ANISOU | 2471 | O | HOH | 52 | 3582 | 4361 | 3489 | 160 | −88 | −116 | O |

| | | | | | | | | | | |
|---|---|---|---|---|---|---|---|---|---|---|
| HETATM | 2472 | O | HOH | 53 | 22.966 | 9.616 | 83.135 | 1.00 | 31.32 | O |

| | | | | | | | | | | | |
|---|---|---|---|---|---|---|---|---|---|---|---|
| ANISOU | 2472 | O | HOH | 53 | 4181 | 3906 | 3815 | 76 | 59 | −1 | O |

| | | | | | | | | | | |
|---|---|---|---|---|---|---|---|---|---|---|
| HETATM | 2473 | O | HOH | 54 | 41.448 | 27.414 | 79.808 | 1.00 | 31.69 | O |

| | | | | | | | | | | | |
|---|---|---|---|---|---|---|---|---|---|---|---|
| ANISOU | 2473 | O | HOH | 54 | 3778 | 3902 | 4360 | 216 | −132 | 74 | O |

| | | | | | | | | | | |
|---|---|---|---|---|---|---|---|---|---|---|
| HETATM | 2474 | O | HOH | 55 | 18.977 | 18.912 | 87.579 | 1.00 | 27.87 | O |

| | | | | | | | | | | | |
|---|---|---|---|---|---|---|---|---|---|---|---|
| ANISOU | 2474 | O | HOH | 55 | 3544 | 3404 | 3643 | −56 | 83 | 537 | O |

| | | | | | | | | | | |
|---|---|---|---|---|---|---|---|---|---|---|
| HETATM | 2475 | O | HOH | 56 | 19.846 | 27.021 | 58.540 | 1.00 | 28.71 | O |

| | | | | | | | | | | | |
|---|---|---|---|---|---|---|---|---|---|---|---|
| ANISOU | 2475 | O | HOH | 56 | 3192 | 4150 | 3565 | −46 | 62 | −102 | O |

| | | | | | | | | | | |
|---|---|---|---|---|---|---|---|---|---|---|
| HETATM | 2476 | O | HOH | 57 | 18.893 | 38.381 | 68.300 | 1.00 | 39.18 | O |

| | | | | | | | | | | | |
|---|---|---|---|---|---|---|---|---|---|---|---|
| ANISOU | 2478 | O | HOH | 57 | 4835 | 5065 | 4988 | −169 | −131 | 302 | O |

| | | | | | | | | | | |
|---|---|---|---|---|---|---|---|---|---|---|
| HETATM | 2477 | O | HOH | 58 | 6.902 | 8.651 | 82.704 | 1.00 | 28.45 | O |

| | | | | | | | | | | | |
|---|---|---|---|---|---|---|---|---|---|---|---|
| ANISOU | 2477 | O | HOH | 58 | 3411 | 3330 | 4067 | −392 | −302 | 113 | O |

| | | | | | | | | | | |
|---|---|---|---|---|---|---|---|---|---|---|
| HETATM | 2478 | O | HOH | 59 | 17.849 | 16.715 | 86.154 | 1.00 | 29.54 | O |

| | | | | | | | | | | | |
|---|---|---|---|---|---|---|---|---|---|---|---|
| ANISOU | 2478 | O | HOH | 59 | 4120 | 3029 | 4077 | −328 | −270 | 384 | O |

| | | | | | | | | | | |
|---|---|---|---|---|---|---|---|---|---|---|
| HETATM | 2479 | O | HOH | 60 | 33.565 | 14.842 | 76.735 | 1.00 | 27.80 | O |

| | | | | | | | | | | | |
|---|---|---|---|---|---|---|---|---|---|---|---|
| ANISOU | 2479 | O | HOH | 60 | 3718 | 2775 | 4069 | 220 | 139 | 28 | O |

| | | | | | | | | | | |
|---|---|---|---|---|---|---|---|---|---|---|
| HETATM | 2480 | O | HOH | 61 | 31.434 | 37.317 | 86.923 | 1.00 | 29.54 | O |

| | | | | | | | | | | | |
|---|---|---|---|---|---|---|---|---|---|---|---|
| ANISOU | 2480 | O | HOH | 61 | 3467 | 4223 | 3531 | −245 | 94 | −71 | O |

| | | | | | | | | | | |
|---|---|---|---|---|---|---|---|---|---|---|
| HETATM | 2481 | O | HOH | 63 | 8.212 | 24.757 | 92.578 | 1.00 | 33.84 | O |

| | | | | | | | | | | | |
|---|---|---|---|---|---|---|---|---|---|---|---|
| ANISOU | 2481 | O | HOH | 63 | 4422 | 4549 | 3887 | −317 | 446 | 186 | O |

| | | | | | | | | | | |
|---|---|---|---|---|---|---|---|---|---|---|
| HETATM | 2482 | O | HOH | 64 | 0.341 | 17.389 | 87.409 | 1.00 | 34.12 | O |

| | | | | | | | | | | | |
|---|---|---|---|---|---|---|---|---|---|---|---|
| ANISOU | 2482 | O | HOH | 64 | 4047 | 4436 | 4479 | 74 | 64 | 41 | O |

| | | | | | | | | | | |
|---|---|---|---|---|---|---|---|---|---|---|
| HETATM | 2483 | O | HOH | 65 | 10.728 | 32.643 | 83.333 | 1.00 | 37.65 | O |

| | | | | | | | | | | | |
|---|---|---|---|---|---|---|---|---|---|---|---|
| ANISOU | 2483 | O | HOH | 65 | 4696 | 4580 | 5031 | 119 | 45 | −165 | O |

| | | | | | | | | | | |
|---|---|---|---|---|---|---|---|---|---|---|
| HETATM | 2484 | O | HOH | 66 | 25.977 | 41.295 | 79.529 | 1.00 | 38.48 | O |

| | | | | | | | | | | | |
|---|---|---|---|---|---|---|---|---|---|---|---|
| ANISOU | 2484 | O | HOH | 66 | 5182 | 4385 | 5052 | 10 | −70 | −13 | O |

| | | | | | | | | | | |
|---|---|---|---|---|---|---|---|---|---|---|
| HETATM | 2485 | O | HOH | 67 | 0.519 | 21.247 | 80.668 | 1.00 | 31.77 | O |

| | | | | | | | | | | | |
|---|---|---|---|---|---|---|---|---|---|---|---|
| ANISOU | 2485 | O | HOH | 67 | 3778 | 4088 | 4207 | −6 | 141 | −23 | O |

| | | | | | | | | | | |
|---|---|---|---|---|---|---|---|---|---|---|
| HETATM | 2486 | O | HOH | 68 | 33.932 | 36.444 | 83.268 | 1.00 | 36.90 | O |

| | | | | | | | | | | | |
|---|---|---|---|---|---|---|---|---|---|---|---|
| ANISOU | 2486 | O | HOH | 68 | 4722 | 4842 | 4454 | 22 | −181 | −382 | O |

| | | | | | | | | | | |
|---|---|---|---|---|---|---|---|---|---|---|
| HETATM | 2487 | O | HOH | 69 | 32.361 | 16.072 | 82.512 | 1.00 | 27.55 | O |

| | | | | | | | | | | | |
|---|---|---|---|---|---|---|---|---|---|---|---|
| ANISOU | 2487 | O | HOH | 69 | 3814 | 3115 | 3538 | 166 | −85 | −130 | O |

| | | | | | | | | | | |
|---|---|---|---|---|---|---|---|---|---|---|
| HETATM | 2488 | O | HOH | 70 | 27.479 | 37.182 | 71.425 | 1.00 | 37.05 | O |

| | | | | | | | | | | | |
|---|---|---|---|---|---|---|---|---|---|---|---|
| ANISOU | 2488 | O | HOH | 70 | 5266 | 4168 | 4644 | −49 | 94 | 223 | O |

| | | | | | | | | | | |
|---|---|---|---|---|---|---|---|---|---|---|
| HETATM | 2489 | O | HOH | 71 | 5.892 | 25.721 | 64.244 | 1.00 | 31.68 | O |

| | | | | | | | | | | | |
|---|---|---|---|---|---|---|---|---|---|---|---|
| ANISOU | 2489 | O | HOH | 71 | 3672 | 4354 | 4009 | −46 | 27 | 158 | O |

| | | | | | | | | | | |
|---|---|---|---|---|---|---|---|---|---|---|
| HETATM | 2490 | O | HOH | 72 | 0.863 | 30.898 | 72.470 | 1.00 | 29.17 | O |

| | | | | | | | | | | | |
|---|---|---|---|---|---|---|---|---|---|---|---|
| ANISOU | 2490 | O | HOH | 72 | 3748 | 3817 | 3519 | 230 | −173 | −465 | O |

| | | | | | | | | | | |
|---|---|---|---|---|---|---|---|---|---|---|
| HETATM | 2491 | O | HOH | 73 | 13.237 | 26.756 | 96.337 | 1.00 | 44.81 | O |

| | | | | | | | | | | | |
|---|---|---|---|---|---|---|---|---|---|---|---|
| ANISOU | 2491 | O | HOH | 73 | 5592 | 5719 | 5716 | −119 | 125 | 4 | O |

| | | | | | | | | | | |
|---|---|---|---|---|---|---|---|---|---|---|
| HETATM | 2492 | O | HOH | 74 | 25.614 | 12.403 | 81.897 | 1.00 | 32.99 | O |

| | | | | | | | | | | | |
|---|---|---|---|---|---|---|---|---|---|---|---|
| ANISOU | 2492 | O | HOH | 74 | 4047 | 4097 | 4391 | 273 | −49 | −168 | O |

| | | | | | | | | | | |
|---|---|---|---|---|---|---|---|---|---|---|
| HETATM | 2493 | O | HOH | 75 | 0.392 | 7.960 | 83.650 | 1.00 | 44.90 | O |

| | | | | | | | | | | | |
|---|---|---|---|---|---|---|---|---|---|---|---|
| ANISOU | 2493 | O | HOH | 75 | 5506 | 5674 | 5881 | −144 | 20 | −128 | O |

| | | | | | | | | | | |
|---|---|---|---|---|---|---|---|---|---|---|
| HETATM | 2494 | O | HOH | 76 | 2.842 | 20.053 | 90.559 | 1.00 | 33.42 | O |

| | | | | | | | | | | | |
|---|---|---|---|---|---|---|---|---|---|---|---|
| ANISOU | 2494 | O | HOH | 76 | 3790 | 4365 | 4544 | −40 | 373 | 370 | O |

| | | | | | | | | | | |
|---|---|---|---|---|---|---|---|---|---|---|
| HETATM | 2495 | O | HOH | 77 | 25.802 | 32.864 | 63.405 | 1.00 | 46.37 | O |

| | | | | | | | | | | | |
|---|---|---|---|---|---|---|---|---|---|---|---|
| ANISOU | 2495 | O | HOH | 77 | 6051 | 5780 | 5787 | −48 | 51 | 212 | O |

| | | | | | | | | | | |
|---|---|---|---|---|---|---|---|---|---|---|
| HETATM | 2496 | O | HOH | 78 | 20.698 | 17.182 | 89.026 | 1.00 | 28.05 | O |

| | | | | | | | | | | | |
|---|---|---|---|---|---|---|---|---|---|---|---|
| ANISOU | 2496 | O | HOH | 78 | 3497 | 3500 | 3659 | 76 | 72 | 370 | O |

| | | | | | | | | | | |
|---|---|---|---|---|---|---|---|---|---|---|
| HETATM | 2497 | O | HOH | 79 | 27.986 | 17.672 | 91.656 | 1.00 | 35.19 | O |

| | | | | | | | | | | | |
|---|---|---|---|---|---|---|---|---|---|---|---|
| ANISOU | 2497 | O | HOH | 79 | 4901 | 3943 | 4528 | 90 | −56 | 268 | O |

| | | | | | | | | | | |
|---|---|---|---|---|---|---|---|---|---|---|
| HETATM | 2498 | O | HOH | 80 | 11.471 | 31.449 | 72.256 | 1.00 | 33.07 | O |

| | | | | | | | | | | | |
|---|---|---|---|---|---|---|---|---|---|---|---|
| ANISOU | 2498 | O | HOH | 80 | 3849 | 4128 | 4589 | −305 | −439 | −386 | O |

| | | | | | | | | | | |
|---|---|---|---|---|---|---|---|---|---|---|
| HETATM | 2499 | O | HOH | 81 | 17.541 | 19.065 | 35.838 | 1.00 | 36.53 | O |

| | | | | | | | | | | | |
|---|---|---|---|---|---|---|---|---|---|---|---|
| ANISOU | 2499 | O | HOH | 81 | 4680 | 4456 | 4742 | −25 | 134 | 234 | O |

| | | | | | | | | | | |
|---|---|---|---|---|---|---|---|---|---|---|
| HETATM | 2500 | O | HOH | 82 | 7.860 | 6.828 | 80.738 | 1.00 | 33.81 | O |

| | | | | | | | | | | | |
|---|---|---|---|---|---|---|---|---|---|---|---|
| ANISOU | 2500 | O | HOH | 82 | 4453 | 3990 | 4403 | −357 | −60 | 289 | O |

| | | | | | | | | | | |
|---|---|---|---|---|---|---|---|---|---|---|
| HETATM | 2501 | O | HOH | 83 | 21.768 | 27.779 | 60.354 | 1.00 | 40.60 | O |

| | | | | | | | | | | | |
|---|---|---|---|---|---|---|---|---|---|---|---|
| ANISOU | 2501 | O | HOH | 83 | 4890 | 5315 | 5222 | 82 | −54 | −47 | O |

| | | | | | | | | | | |
|---|---|---|---|---|---|---|---|---|---|---|
| HETATM | 2502 | O | HOH | 84 | 32.064 | 37.367 | 89.481 | 1.00 | 41.76 | O |

| | | | | | | | | | | | |
|---|---|---|---|---|---|---|---|---|---|---|---|
| ANISOU | 2502 | O | HOH | 84 | 5354 | 5268 | 5244 | −7 | −99 | 147 | O |

| | | | | | | | | | | |
|---|---|---|---|---|---|---|---|---|---|---|
| HETATM | 2503 | O | HOH | 85 | 8.503 | 9.586 | 67.648 | 1.00 | 35.02 | O |

| | | | | | | | | | | | |
|---|---|---|---|---|---|---|---|---|---|---|---|
| ANISOU | 2503 | O | HOH | 85 | 4560 | 4198 | 4548 | −151 | −92 | −68 | O |

| | | | | | | | | | | |
|---|---|---|---|---|---|---|---|---|---|---|
| HETATM | 2504 | O | HOH | 86 | 35.055 | 16.029 | 80.808 | 1.00 | 30.32 | O |

| | | | | | | | | | | | |
|---|---|---|---|---|---|---|---|---|---|---|---|
| ANISOU | 2504 | O | HOH | 86 | 4584 | 3294 | 3640 | −45 | 233 | −30 | O |

| | | | | | | | | | | |
|---|---|---|---|---|---|---|---|---|---|---|
| HETATM | 2505 | O | HOH | 87 | 6.989 | 13.177 | 66.266 | 1.00 | 33.80 | O |

| | | | | | | | | | | | |
|---|---|---|---|---|---|---|---|---|---|---|---|
| ANISOU | 2505 | O | HOH | 87 | 4125 | 3818 | 4898 | −251 | −63 | −11 | O |

| | | | | | | | | | | |
|---|---|---|---|---|---|---|---|---|---|---|
| HETATM | 2506 | O | HOH | 88 | 15.003 | 28.045 | 97.998 | 1.00 | 51.17 | O |

| | | | | | | | | | | | |
|---|---|---|---|---|---|---|---|---|---|---|---|
| ANISOU | 2506 | O | HOH | 88 | 6357 | 6627 | 6457 | 16 | −15 | −2 | O |

| | | | | | | | | | | |
|---|---|---|---|---|---|---|---|---|---|---|
| HETATM | 2507 | O | HOH | 89 | 29.907 | 36.459 | 67.348 | 1.00 | 31.26 | O |

| | | | | | | | | | | | |
|---|---|---|---|---|---|---|---|---|---|---|---|
| ANISOU | 2507 | O | HOH | 89 | 4265 | 3612 | 3999 | −117 | 198 | −44 | O |

| | | | | | | | | | | |
|---|---|---|---|---|---|---|---|---|---|---|
| HETATM | 2508 | O | HOH | 90 | 10.674 | 27.500 | 42.469 | 1.00 | 43.25 | O |

| | | | | | | | | | | | |
|---|---|---|---|---|---|---|---|---|---|---|---|
| ANISOU | 2508 | O | HOH | 90 | 5645 | 5598 | 5190 | 95 | 86 | −41 | O |

| | | | | | | | | | | |
|---|---|---|---|---|---|---|---|---|---|---|
| HETATM | 2509 | O | HOH | 92 | 20.274 | 18.178 | 70.165 | 1.00 | 30.26 | O |

| | | | | | | | | | | | |
|---|---|---|---|---|---|---|---|---|---|---|---|
| ANISOU | 2509 | O | HOH | 92 | 3601 | 4289 | 3606 | −159 | 23 | −276 | O |

| | | | | | | | | | | |
|---|---|---|---|---|---|---|---|---|---|---|
| HETATM | 2510 | O | HOH | 93 | 32.384 | 22.940 | 91.171 | 1.00 | 32.02 | O |

| | | | | | | | | | | | |
|---|---|---|---|---|---|---|---|---|---|---|---|
| ANISOU | 2510 | O | HOH | 93 | 3973 | 4057 | 4137 | −113 | −116 | 510 | O |

| | | | | | | | | | | |
|---|---|---|---|---|---|---|---|---|---|---|
| HETATM | 2511 | O | HOH | 94 | 12.207 | 36.458 | 58.100 | 1.00 | 45.11 | O |

| | | | | | | | | | | | |
|---|---|---|---|---|---|---|---|---|---|---|---|
| ANISOU | 2511 | O | HOH | 94 | 5761 | 5563 | 5814 | −38 | 46 | −39 | O |

| | | | | | | | | | | |
|---|---|---|---|---|---|---|---|---|---|---|
| HETATM | 2512 | O | HOH | 95 | 27.430 | 27.136 | 93.699 | 1.00 | 40.85 | O |

| | | | | | | | | | | | |
|---|---|---|---|---|---|---|---|---|---|---|---|
| ANISOU | 2512 | O | HOH | 95 | 5177 | 5242 | 5102 | 21 | 50 | 42 | O |

| | | | | | | | | | | |
|---|---|---|---|---|---|---|---|---|---|---|
| HETATM | 2513 | O | HOH | 97 | 37.831 | 29.448 | 86.376 | 1.00 | 30.88 | O |

| | | | | | | | | | | | |
|---|---|---|---|---|---|---|---|---|---|---|---|
| ANISOU | 2513 | O | HOH | 97 | 4017 | 3710 | 4008 | −175 | −67 | 35 | O |

| | | | | | | | | | | |
|---|---|---|---|---|---|---|---|---|---|---|
| HETATM | 2514 | O | HOH | 98 | 15.240 | 18.925 | 66.709 | 1.00 | 38.00 | O |

| | | | | | | | | | | | |
|---|---|---|---|---|---|---|---|---|---|---|---|
| ANISOU | 2514 | O | HOH | 98 | 4994 | 4738 | 4706 | −7 | −242 | 176 | O |

| | | | | | | | | | | |
|---|---|---|---|---|---|---|---|---|---|---|
| HETATM | 2515 | O | HOH | 99 | 5.829 | 19.721 | 64.048 | 1.00 | 35.79 | O |

| | | | | | | | | | | | |
|---|---|---|---|---|---|---|---|---|---|---|---|
| ANISOU | 2515 | O | HOH | 99 | 4801 | 4416 | 4380 | −274 | 19 | 66 | O |

| | | | | | | | | | | |
|---|---|---|---|---|---|---|---|---|---|---|
| HETATM | 2516 | O | HOH | 101 | 6.090 | 6.593 | 72.934 | 1.00 | 39.21 | O |

| | | | | | | | | | | | |
|---|---|---|---|---|---|---|---|---|---|---|---|
| ANISOU | 2516 | O | HOH | 101 | 5096 | 4609 | 5195 | −199 | −38 | −118 | O |

| | | | | | | | | | | |
|---|---|---|---|---|---|---|---|---|---|---|
| HETATM | 2517 | O | HOH | 102 | 26.208 | 34.093 | 92.270 | 1.00 | 34.48 | O |

| | | | | | | | | | | | |
|---|---|---|---|---|---|---|---|---|---|---|---|
| ANISOU | 2517 | O | HOH | 102 | 4658 | 4274 | 4169 | 10 | −114 | −190 | O |

| | | | | | | | | | | |
|---|---|---|---|---|---|---|---|---|---|---|
| HETATM | 2518 | O | HOH | 103 | 19.304 | 37.782 | 81.584 | 1.00 | 42.00 | O |

| | | | | | | | | | | | |
|---|---|---|---|---|---|---|---|---|---|---|---|
| ANISOU | 2518 | O | HOH | 103 | 5191 | 5317 | 5451 | 114 | 35 | O | O |

| | | | | | | | | | | |
|---|---|---|---|---|---|---|---|---|---|---|
| HETATM | 2519 | O | HOH | 104 | 10.583 | 32.305 | 86.587 | 1.00 | 39.73 | O |

| | | | | | | | | | | | |
|---|---|---|---|---|---|---|---|---|---|---|---|
| ANISOU | 2519 | O | HOH | 104 | 4880 | 4948 | 5265 | 115 | 208 | −302 | O |

| | | | | | | | | | | |
|---|---|---|---|---|---|---|---|---|---|---|
| HETATM | 2520 | O | HOH | 105 | 4.086 | 7.006 | 74.602 | 1.00 | 42.19 | O |

| | | | | | | | | | | | |
|---|---|---|---|---|---|---|---|---|---|---|---|
| ANISOU | 2520 | O | HOH | 105 | 5191 | 5231 | 5610 | −135 | −123 | −43 | O |

| | | | | | | | | | | |
|---|---|---|---|---|---|---|---|---|---|---|
| HETATM | 2521 | O | HOH | 106 | 36.699 | 40.392 | 83.292 | 1.00 | 44.11 | O |

| | | | | | | | | | | | |
|---|---|---|---|---|---|---|---|---|---|---|---|
| ANISOU | 2521 | O | HOH | 106 | 5779 | 5365 | 5616 | 119 | 20 | 287 | O |

| | | | | | | | | | | |
|---|---|---|---|---|---|---|---|---|---|---|
| HETATM | 2522 | O | HOH | 107 | 17.276 | 37.421 | 90.299 | 1.00 | 40.27 | O |

| | | | | | | | | | | | |
|---|---|---|---|---|---|---|---|---|---|---|---|
| ANISOU | 2522 | O | HOH | 107 | 5148 | 4767 | 5385 | −33 | −7 | −97 | O |

| | | | | | | | | | | |
|---|---|---|---|---|---|---|---|---|---|---|
| HETATM | 2523 | O | HOH | 108 | 30.148 | 37.356 | 69.897 | 1.00 | 35.36 | O |

| | | | | | | | | | | | |
|---|---|---|---|---|---|---|---|---|---|---|---|
| ANISOU | 2523 | O | HOH | 108 | 4787 | 4293 | 4354 | −46 | 184 | 242 | O |

| | | | | | | | | | | |
|---|---|---|---|---|---|---|---|---|---|---|
| HETATM | 2524 | O | HOH | 109 | 20.050 | 11.270 | 82.593 | 1.00 | 30.97 | O |

| | | | | | | | | | | | |
|---|---|---|---|---|---|---|---|---|---|---|---|
| ANISOU | 2524 | O | HOH | 109 | 3907 | 3736 | 4126 | −397 | −192 | −331 | O |

| | | | | | | | | | | |
|---|---|---|---|---|---|---|---|---|---|---|
| HETATM | 2525 | O | HOH | 110 | 32.982 | 18.621 | 61.866 | 1.00 | 40.10 | O |

| | | | | | | | | | | | |
|---|---|---|---|---|---|---|---|---|---|---|---|
| ANISOU | 2525 | O | HOH | 110 | 5006 | 5145 | 5083 | −75 | −7 | −109 | O |

| | | | | | | | | | | |
|---|---|---|---|---|---|---|---|---|---|---|
| HETATM | 2526 | O | HOH | 111 | 31.183 | 44.426 | 71.705 | 1.00 | 38.92 | O |

| | | | | | | | | | | | |
|---|---|---|---|---|---|---|---|---|---|---|---|
| ANISOU | 2526 | O | HOH | 111 | 5834 | 4043 | 4912 | 117 | −199 | −112 | O |

| | | | | | | | | | | |
|---|---|---|---|---|---|---|---|---|---|---|
| HETATM | 2527 | O | HOH | 112 | 19.410 | 35.437 | 91.026 | 1.00 | 31.27 | O |

| | | | | | | | | | | | |
|---|---|---|---|---|---|---|---|---|---|---|---|
| ANISOU | 2527 | O | HOH | 112 | 4105 | 3674 | 4102 | 20 | −307 | −1 | O |

| | | | | | | | | | | |
|---|---|---|---|---|---|---|---|---|---|---|
| HETATM | 2528 | O | HOH | 113 | 10.508 | 34.816 | 74.376 | 1.00 | 35.06 | O |

| | | | | | | | | | | | |
|---|---|---|---|---|---|---|---|---|---|---|---|
| ANISOU | 2528 | O | HOH | 113 | 4113 | 4123 | 5085 | 46 | −303 | 5 | O |

| | | | | | | | | | | |
|---|---|---|---|---|---|---|---|---|---|---|
| HETATM | 2529 | O | HOH | 114 | 1.049 | 11.092 | 82.134 | 1.00 | 40.12 | O |

| | | | | | | | | | | | |
|---|---|---|---|---|---|---|---|---|---|---|---|
| ANISOU | 2529 | O | HOH | 114 | 4844 | 4782 | 5618 | −315 | 69 | −108 | O |

| | | | | | | | | | | |
|---|---|---|---|---|---|---|---|---|---|---|
| HETATM | 2530 | O | HOH | 115 | 19.897 | 5.671 | 74.532 | 1.00 | 36.57 | O |

| | | | | | | | | | | | |
|---|---|---|---|---|---|---|---|---|---|---|---|
| ANISOU | 2530 | O | HOH | 115 | 5109 | 4121 | 4665 | 66 | 99 | −313 | O |

| | | | | | | | | | | |
|---|---|---|---|---|---|---|---|---|---|---|
| HETATM | 2531 | O | HOH | 116 | 12.797 | 36.998 | 68.353 | 1.00 | 44.20 | O |

| | | | | | | | | | | | |
|---|---|---|---|---|---|---|---|---|---|---|---|
| ANISOU | 2531 | O | HOH | 116 | 5407 | 5654 | 5732 | −9 | 11 | −23 | O |

| | | | | | | | | | | |
|---|---|---|---|---|---|---|---|---|---|---|
| HETATM | 2532 | O | HOH | 117 | 37.204 | 16.913 | 77.312 | 1.00 | 42.42 | O |

| | | | | | | | | | | | |
|---|---|---|---|---|---|---|---|---|---|---|---|
| ANISOU | 2532 | O | HOH | 117 | 5547 | 5241 | 5330 | 35 | 136 | −64 | O |

| | | | | | | | | | | |
|---|---|---|---|---|---|---|---|---|---|---|
| HETATM | 2533 | O | HOH | 118 | 24.098 | 26.614 | 55.883 | 1.00 | 46.04 | O |

| | | | | | | | | | | | |
|---|---|---|---|---|---|---|---|---|---|---|---|
| ANISOU | 2533 | O | HOH | 118 | 5820 | 5869 | 5803 | −112 | −31 | 92 | O |

| | | | | | | | | | | |
|---|---|---|---|---|---|---|---|---|---|---|
| HETATM | 2534 | O | HOH | 119 | 9.740 | 7.380 | 78.924 | 1.00 | 35.49 | O |

| | | | | | | | | | | | |
|---|---|---|---|---|---|---|---|---|---|---|---|
| ANISOU | 2534 | O | HOH | 119 | 4494 | 4049 | 4943 | −286 | −98 | −193 | O |

| | | | | | | | | | | |
|---|---|---|---|---|---|---|---|---|---|---|
| HETATM | 2535 | O | HOH | 120 | 26.325 | 10.188 | 80.789 | 1.00 | 36.65 | O |

| | | | | | | | | | | | |
|---|---|---|---|---|---|---|---|---|---|---|---|
| ANISOU | 2535 | O | HOH | 120 | 4396 | 4867 | 4661 | −85 | 174 | −265 | O |

| | | | | | | | | | | |
|---|---|---|---|---|---|---|---|---|---|---|
| HETATM | 2536 | O | HOH | 121 | 24.800 | 8.376 | 81.628 | 1.00 | 36.20 | O |

| | | | | | | | | | | | |
|---|---|---|---|---|---|---|---|---|---|---|---|
| ANISOU | 2536 | O | HOH | 121 | 4413 | 4540 | 4803 | 33 | −114 | −123 | O |

| | | | | | | | | | | |
|---|---|---|---|---|---|---|---|---|---|---|
| HETATM | 2537 | O | HOH | 122 | 0.529 | 25.300 | 65.097 | 1.00 | 30.84 | O |

| | | | | | | | | | | | |
|---|---|---|---|---|---|---|---|---|---|---|---|
| ANISOU | 2537 | O | HOH | 122 | 4482 | 3825 | 3412 | −21 | 85 | 5 | O |

| | | | | | | | | | | |
|---|---|---|---|---|---|---|---|---|---|---|
| HETATM | 2538 | O | HOH | 123 | 16.009 | 22.094 | 95.421 | 1.00 | 37.78 | O |

| | | | | | | | | | | | |
|---|---|---|---|---|---|---|---|---|---|---|---|
| ANISOU | 2538 | O | HOH | 123 | 4667 | 5104 | 4586 | −25 | 39 | 87 | O |

| | | | | | | | | | | |
|---|---|---|---|---|---|---|---|---|---|---|
| HETATM | 2539 | O | HOH | 124 | 42.684 | 19.628 | 77.757 | 1.00 | 34.07 | O |

| | | | | | | | | | | | |
|---|---|---|---|---|---|---|---|---|---|---|---|
| ANISOU | 2539 | O | HOH | 124 | 4309 | 4247 | 4388 | 254 | −218 | 92 | O |

| | | | | | | | | | | |
|---|---|---|---|---|---|---|---|---|---|---|
| HETATM | 2540 | O | HOH | 125 | 35.783 | 23.733 | 86.601 | 1.00 | 36.64 | O |

| | | | | | | | | | | | |
|---|---|---|---|---|---|---|---|---|---|---|---|
| ANISOU | 2540 | O | HOH | 125 | 4533 | 4858 | 4530 | 67 | −40 | −85 | O |

| | | | | | | | | | | |
|---|---|---|---|---|---|---|---|---|---|---|
| HETATM | 2541 | O | HOH | 126 | 21.796 | 40.661 | 81.020 | 1.00 | 29.22 | O |

| | | | | | | | | | | | |
|---|---|---|---|---|---|---|---|---|---|---|---|
| ANISOU | 2541 | O | HOH | 126 | 4192 | 3615 | 3294 | 121 | −111 | −252 | O |

| | | | | | | | | | | |
|---|---|---|---|---|---|---|---|---|---|---|
| HETATM | 2542 | O | HOH | 127 | 19.560 | 40.153 | 79.738 | 1.00 | 34.02 | O |

| | | | | | | | | | | | |
|---|---|---|---|---|---|---|---|---|---|---|---|
| ANISOU | 2542 | O | HOH | 127 | 4065 | 4328 | 4535 | 11 | 243 | −240 | O |

| | | | | | | | | | | |
|---|---|---|---|---|---|---|---|---|---|---|
| HETATM | 2543 | O | HOH | 128 | 24.074 | 40.355 | 87.308 | 1.00 | 36.13 | O |

| | | | | | | | | | | | |
|---|---|---|---|---|---|---|---|---|---|---|---|
| ANISOU | 2543 | O | HOH | 128 | 4930 | 4116 | 4679 | −9 | 40 | −138 | O |

| | | | | | | | | | | |
|---|---|---|---|---|---|---|---|---|---|---|
| HETATM | 2544 | O | HOH | 129 | 9.039 | 32.249 | 66.343 | 1.00 | 32.45 | O |

| | | | | | | | | | | | |
|---|---|---|---|---|---|---|---|---|---|---|---|
| ANISOU | 2544 | O | HOH | 129 | 3876 | 4369 | 4085 | 79 | 91 | 152 | O |

| | | | | | | | | | | |
|---|---|---|---|---|---|---|---|---|---|---|
| HETATM | 2545 | O | HOH | 130 | 12.440 | 34.199 | 86.784 | 1.00 | 39.42 | O |

| | | | | | | | | | | | |
|---|---|---|---|---|---|---|---|---|---|---|---|
| ANISOU | 2545 | O | HOH | 130 | 4746 | 5219 | 5013 | 65 | 187 | −163 | O |

| | | | | | | | | | | |
|---|---|---|---|---|---|---|---|---|---|---|
| HETATM | 2546 | O | HOH | 132 | 18.577 | 41.151 | 72.469 | 1.00 | 40.69 | O |

| | | | | | | | | | | | |
|---|---|---|---|---|---|---|---|---|---|---|---|
| ANISOU | 2546 | O | HOH | 132 | 5132 | 5116 | 5212 | −131 | 38 | 175 | O |

| | | | | | | | | | | |
|---|---|---|---|---|---|---|---|---|---|---|
| HETATM | 2547 | O | HOH | 133 | 26.035 | 17.547 | 59.645 | 1.00 | 37.43 | O |

| | | | | | | | | | | | |
|---|---|---|---|---|---|---|---|---|---|---|---|
| ANISOU | 2547 | O | HOH | 133 | 4588 | 4901 | 4733 | 41 | −7 | −48 | O |

| | | | | | | | | | | |
|---|---|---|---|---|---|---|---|---|---|---|
| HETATM | 2548 | O | HOH | 134 | 6.841 | 27.863 | 88.767 | 1.00 | 43.13 | O |

| | | | | | | | | | | | |
|---|---|---|---|---|---|---|---|---|---|---|---|
| ANISOU | 2548 | O | HOH | 134 | 5586 | 5488 | 5312 | −99 | −198 | 85 | O |

| | | | | | | | | | | |
|---|---|---|---|---|---|---|---|---|---|---|
| HETATM | 2549 | O | HOH | 135 | 5.747 | 24.462 | 60.265 | 1.00 | 31.64 | O |

| | | | | | | | | | | | |
|---|---|---|---|---|---|---|---|---|---|---|---|
| ANISOU | 2549 | O | HOH | 135 | 3794 | 4365 | 3864 | −157 | −132 | −29 | O |

| | | | | | | | | | | |
|---|---|---|---|---|---|---|---|---|---|---|
| HETATM | 2550 | O | HOH | 136 | 5.347 | 31.806 | 46.511 | 1.00 | 34.07 | O |

| | | | | | | | | | | | |
|---|---|---|---|---|---|---|---|---|---|---|---|
| ANISOU | 2550 | O | HOH | 136 | 4493 | 4356 | 4098 | 306 | −297 | 88 | O |

| | | | | | | | | | | |
|---|---|---|---|---|---|---|---|---|---|---|
| HETATM | 2551 | O | HOH | 137 | 22.102 | 8.719 | 85.540 | 1.00 | 35.29 | O |

| | | | | | | | | | | | |
|---|---|---|---|---|---|---|---|---|---|---|---|
| ANISOU | 2551 | O | HOH | 137 | 4212 | 4737 | 4457 | −50 | 118 | −7 | O |

| | | | | | | | | | | |
|---|---|---|---|---|---|---|---|---|---|---|
| HETATM | 2552 | O | HOH | 138 | 21.348 | 33.060 | 45.448 | 1.00 | 50.48 | O |

| | | | | | | | | | | | |
|---|---|---|---|---|---|---|---|---|---|---|---|
| ANISOU | 2552 | O | HOH | 138 | 6358 | 6409 | 6412 | −24 | 89 | −14 | O |

| | | | | | | | | | | |
|---|---|---|---|---|---|---|---|---|---|---|
| HETATM | 2553 | O | HOH | 139 | 21.196 | 38.672 | 66.903 | 1.00 | 38.48 | O |

| | | | | | | | | | | | |
|---|---|---|---|---|---|---|---|---|---|---|---|
| ANISOU | 2553 | O | HOH | 139 | 4950 | 4534 | 5139 | −44 | 180 | 282 | O |

| | | | | | | | | | | |
|---|---|---|---|---|---|---|---|---|---|---|
| HETATM | 2554 | O | HOH | 140 | 19.595 | 17.890 | 37.318 | 1.00 | 42.21 | O |

| | | | | | | | | | | | |
|---|---|---|---|---|---|---|---|---|---|---|---|
| ANISOU | 2554 | O | HOH | 140 | 5649 | 5133 | 5254 | −91 | 9 | 18 | O |

| | | | | | | | | | | |
|---|---|---|---|---|---|---|---|---|---|---|
| HETATM | 2555 | O | HOH | 141 | 17.506 | 17.570 | 66.106 | 1.00 | 46.57 | O |

| | | | | | | | | | | | |
|---|---|---|---|---|---|---|---|---|---|---|---|
| ANISOU | 2555 | O | HOH | 141 | 5703 | 6043 | 5950 | −156 | −14 | −25 | O |

| | | | | | | | | | | |
|---|---|---|---|---|---|---|---|---|---|---|
| HETATM | 2556 | O | HOH | 142 | 37.768 | 20.451 | 86.955 | 1.00 | 39.70 | O |

| | | | | | | | | | | | |
|---|---|---|---|---|---|---|---|---|---|---|---|
| ANISOU | 2556 | O | HOH | 142 | 4699 | 5282 | 5103 | 57 | −225 | 95 | O |

| | | | | | | | | | | |
|---|---|---|---|---|---|---|---|---|---|---|
| HETATM | 2557 | O | HOH | 144 | 20.596 | 25.321 | 45.223 | 1.00 | 40.73 | O |

| | | | | | | | | | | | |
|---|---|---|---|---|---|---|---|---|---|---|---|
| ANISOU | 2557 | O | HOH | 144 | 5073 | 5370 | 5033 | 60 | 83 | 12 | O |

| | | | | | | | | | | |
|---|---|---|---|---|---|---|---|---|---|---|
| HETATM | 2558 | O | HOH | 145 | 24.915 | 20.661 | 59.526 | 1.00 | 32.65 | O |

| | | | | | | | | | | | |
|---|---|---|---|---|---|---|---|---|---|---|---|
| ANISOU | 2558 | O | HOH | 145 | 4431 | 4051 | 3925 | −44 | −70 | −36 | O |

| | | | | | | | | | | |
|---|---|---|---|---|---|---|---|---|---|---|
| HETATM | 2559 | O | HOH | 146 | 23.130 | 25.120 | 59.370 | 1.00 | 32.05 | O |

| | | | | | | | | | | | |
|---|---|---|---|---|---|---|---|---|---|---|---|
| ANISOU | 2559 | O | HOH | 146 | 3775 | 4339 | 4063 | 220 | 207 | 83 | O |

| | | | | | | | | | | |
|---|---|---|---|---|---|---|---|---|---|---|
| HETATM | 2560 | O | HOH | 147 | 15.211 | 22.046 | 92.930 | 1.00 | 38.03 | O |

| | | | | | | | | | | | |
|---|---|---|---|---|---|---|---|---|---|---|---|
| ANISOU | 2560 | O | HOH | 147 | 4653 | 4976 | 4819 | 5 | −16 | 221 | O |

| | | | | | | | | | | |
|---|---|---|---|---|---|---|---|---|---|---|
| HETATM | 2561 | O | HOH | 148 | 3.874 | 5.766 | 77.070 | 1.00 | 45.93 | O |

| | | | | | | | | | | | |
|---|---|---|---|---|---|---|---|---|---|---|---|
| ANISOU | 2561 | O | HOH | 148 | 5735 | 5794 | 5921 | 146 | −74 | 72 | O |

| | | | | | | | | | | |
|---|---|---|---|---|---|---|---|---|---|---|
| HETATM | 2562 | O | HOH | 149 | 39.692 | 16.148 | 81.822 | 1.00 | 42.50 | O |

| | | | | | | | | | | | |
|---|---|---|---|---|---|---|---|---|---|---|---|
| ANISOU | 2562 | O | HOH | 149 | 5244 | 5204 | 5700 | 150 | −53 | 7 | O |

| | | | | | | | | | | |
|---|---|---|---|---|---|---|---|---|---|---|
| HETATM | 2563 | O | HOH | 150 | 28.544 | 29.691 | 94.309 | 1.00 | 45.60 | O |

| | | | | | | | | | | | |
|---|---|---|---|---|---|---|---|---|---|---|---|
| ANISOU | 2563 | O | HOH | 150 | 5856 | 5761 | 5709 | −69 | 12 | 11 | O |

| | | | | | | | | | | |
|---|---|---|---|---|---|---|---|---|---|---|
| HETATM | 2564 | O | HOH | 151 | 15.761 | 28.212 | 88.984 | 1.00 | 33.46 | O |

| | | | | | | | | | | | |
|---|---|---|---|---|---|---|---|---|---|---|---|
| ANISOU | 2564 | O | HOH | 151 | 4658 | 4153 | 3901 | −163 | 268 | 362 | O |

| | | | | | | | | | | |
|---|---|---|---|---|---|---|---|---|---|---|
| HETATM | 2565 | O | HOH | 152 | 3.456 | 35.442 | 52.303 | 1.00 | 40.94 | O |

| | | | | | | | | | | | |
|---|---|---|---|---|---|---|---|---|---|---|---|
| ANISOU | 2565 | O | HOH | 152 | 5075 | 5208 | 5272 | 260 | −207 | 13 | O |

| | | | | | | | | | | |
|---|---|---|---|---|---|---|---|---|---|---|
| HETATM | 2566 | O | HOH | 153 | 38.432 | 31.309 | 63.732 | 1.00 | 54.84 | O |

| | | | | | | | | | | | |
|---|---|---|---|---|---|---|---|---|---|---|---|
| ANISOU | 2566 | O | HOH | 153 | 6983 | 6977 | 6877 | 87 | −54 | −132 | O |

| | | | | | | | | | | |
|---|---|---|---|---|---|---|---|---|---|---|
| HETATM | 2567 | O | HOH | 154 | 45.567 | 30.031 | 75.704 | 1.00 | 48.83 | O |

| | | | | | | | | | | | |
|---|---|---|---|---|---|---|---|---|---|---|---|
| ANISOU | 2567 | O | HOH | 154 | 6039 | 6212 | 6304 | −60 | 37 | −69 | O |

| | | | | | | | | | | |
|---|---|---|---|---|---|---|---|---|---|---|
| HETATM | 2568 | O | HOH | 155 | 5.351 | 7.890 | 70.467 | 1.00 | 34.90 | O |

| | | | | | | | | | | | |
|---|---|---|---|---|---|---|---|---|---|---|---|
| ANISOU | 2568 | O | HOH | 155 | 4391 | 4202 | 4666 | −41 | −141 | −86 | O |

| | | | | | | | | | | |
|---|---|---|---|---|---|---|---|---|---|---|
| HETATM | 2569 | O | HOH | 156 | 5.248 | 12.467 | 63.713 | 1.00 | 49.93 | O |

| | | | | | | | | | | | |
|---|---|---|---|---|---|---|---|---|---|---|---|
| ANISOU | 2569 | O | HOH | 156 | 6398 | 6210 | 6364 | −98 | −91 | −20 | O |

| | | | | | | | | | | |
|---|---|---|---|---|---|---|---|---|---|---|
| HETATM | 2570 | O | HOH | 157 | 19.804 | 12.771 | 80.652 | 1.00 | 38.88 | O |

| | | | | | | | | | | | |
|---|---|---|---|---|---|---|---|---|---|---|---|
| ANISOU | 2570 | O | HOH | 157 | 4912 | 4876 | 4986 | −111 | −2 | −195 | O |

| | | | | | | | | | | |
|---|---|---|---|---|---|---|---|---|---|---|
| HETATM | 2571 | O | HOH | 158 | 24.298 | 27.085 | 62.174 | 1.00 | 37.61 | O |

| | | | | | | | | | | | |
|---|---|---|---|---|---|---|---|---|---|---|---|
| ANISOU | 2571 | O | HOH | 158 | 5032 | 4699 | 4558 | 209 | 198 | 5 | O |

| | | | | | | | | | | |
|---|---|---|---|---|---|---|---|---|---|---|
| HETATM | 2572 | O | HOH | 159 | −2.754 | 18.937 | 70.168 | 1.00 | 49.15 | O |

| | | | | | | | | | | | |
|---|---|---|---|---|---|---|---|---|---|---|---|
| ANISOU | 2572 | O | HOH | 159 | 6055 | 6297 | 6322 | 135 | −27 | −66 | O |

| | | | | | | | | | | |
|---|---|---|---|---|---|---|---|---|---|---|
| HETATM | 2573 | O | HOH | 160 | 20.325 | 9.737 | 74.178 | 1.00 | 33.76 | O |

| | | | | | | | | | | | |
|---|---|---|---|---|---|---|---|---|---|---|---|
| ANISOU | 2573 | O | HOH | 160 | 4095 | 4137 | 4594 | −44 | 180 | −237 | O |

| | | | | | | | | | | |
|---|---|---|---|---|---|---|---|---|---|---|
| HETATM | 2574 | O | HOH | 161 | 4.663 | 27.190 | 59.648 | 1.00 | 44.33 | O |

| | | | | | | | | | | | |
|---|---|---|---|---|---|---|---|---|---|---|---|
| ANISOU | 2574 | O | HOH | 161 | 5691 | 5924 | 5227 | 128 | −4 | 114 | O |

| | | | | | | | | | | |
|---|---|---|---|---|---|---|---|---|---|---|
| HETATM | 2575 | O | HOH | 162 | 36.688 | 13.560 | 67.532 | 1.00 | 45.15 | O |

| | | | | | | | | | | | |
|---|---|---|---|---|---|---|---|---|---|---|---|
| ANISOU | 2575 | O | HOH | 162 | 5819 | 5609 | 5729 | −39 | 5 | 64 | O |

| | | | | | | | | | | |
|---|---|---|---|---|---|---|---|---|---|---|
| HETATM | 2576 | O | HOH | 163 | 17.051 | 29.122 | 91.358 | 1.00 | 43.44 | O |

| | | | | | | | | | | | |
|---|---|---|---|---|---|---|---|---|---|---|---|
| ANISOU | 2576 | O | HOH | 163 | 5359 | 5484 | 5663 | −185 | 60 | 22 | O |

| | | | | | | | | | | |
|---|---|---|---|---|---|---|---|---|---|---|
| HETATM | 2577 | O | HOH | 164 | 19.304 | 17.699 | 64.312 | 1.00 | 34.62 | O |

| | | | | | | | | | | | |
|---|---|---|---|---|---|---|---|---|---|---|---|
| ANISOU | 2577 | O | HOH | 164 | 4486 | 4139 | 4530 | 8 | 46 | −91 | O |

| | | | | | | | | | | |
|---|---|---|---|---|---|---|---|---|---|---|
| HETATM | 2578 | O | HOH | 165 | 4.437 | 33.730 | 48.186 | 1.00 | 44.78 | O |

| | | | | | | | | | | | |
|---|---|---|---|---|---|---|---|---|---|---|---|
| ANISOU | 2578 | O | HOH | 165 | 5530 | 5789 | 5697 | 192 | −103 | 47 | O |

| | | | | | | | | | | |
|---|---|---|---|---|---|---|---|---|---|---|
| HETATM | 2579 | O | HOH | 166 | 19.582 | 15.633 | 67.366 | 1.00 | 39.27 | O |

| | | | | | | | | | | | |
|---|---|---|---|---|---|---|---|---|---|---|---|
| ANISOU | 2579 | O | HOH | 166 | 5095 | 4943 | 4884 | 41 | 307 | −86 | O |

| | | | | | | | | | | |
|---|---|---|---|---|---|---|---|---|---|---|
| HETATM | 2580 | O | HOH | 167 | 3.313 | 25.443 | 63.514 | 1.00 | 35.90 | O |

| | | | | | | | | | | | |
|---|---|---|---|---|---|---|---|---|---|---|---|
| ANISOU | 2580 | O | HOH | 167 | 4498 | 4592 | 4549 | 4 | −51 | 116 | O |

| | | | | | | | | | | |
|---|---|---|---|---|---|---|---|---|---|---|
| HETATM | 2581 | O | HOH | 168 | 29.036 | 33.803 | 92.215 | 1.00 | 37.42 | O |

| | | | | | | | | | | | |
|---|---|---|---|---|---|---|---|---|---|---|---|
| ANISOU | 2581 | O | HOH | 168 | 4880 | 4260 | 5077 | 229 | −77 | 84 | O |

| | | | | | | | | | | |
|---|---|---|---|---|---|---|---|---|---|---|
| HETATM | 2582 | O | HOH | 169 | 27.439 | 37.535 | 67.031 | 1.00 | 49.94 | O |

| | | | | | | | | | | | |
|---|---|---|---|---|---|---|---|---|---|---|---|
| ANISOU | 2582 | O | HOH | 169 | 6490 | 6199 | 6285 | 67 | 81 | 45 | O |

| | | | | | | | | | | |
|---|---|---|---|---|---|---|---|---|---|---|
| HETATM | 2583 | O | HOH | 170 | 39.359 | 22.141 | 64.730 | 1.00 | 39.59 | O |

| | | | | | | | | | | | |
|---|---|---|---|---|---|---|---|---|---|---|---|
| ANISOU | 2583 | O | HOH | 170 | 4994 | 4958 | 5090 | −66 | 172 | 89 | O |

| | | | | | | | | | | |
|---|---|---|---|---|---|---|---|---|---|---|
| HETATM | 2584 | O | HOH | 171 | 2.618 | 29.140 | 76.067 | 1.00 | 40.90 | O |

| | | | | | | | | | | | |
|---|---|---|---|---|---|---|---|---|---|---|---|
| ANISOU | 2584 | O | HOH | 171 | 5201 | 5034 | 5304 | 72 | −24 | 42 | O |

| | | | | | | | | | | |
|---|---|---|---|---|---|---|---|---|---|---|
| HETATM | 2585 | O | HOH | 172 | 19.141 | 42.727 | 78.598 | 1.00 | 44.77 | O |

| | | | | | | | | | | | |
|---|---|---|---|---|---|---|---|---|---|---|---|
| ANISOU | 2585 | O | HOH | 172 | 5768 | 5454 | 5789 | 104 | 93 | −108 | O |

| | | | | | | | | | | |
|---|---|---|---|---|---|---|---|---|---|---|
| HETATM | 2586 | O | HOH | 173 | 26.708 | 12.693 | 66.306 | 1.00 | 51.60 | O |

| | | | | | | | | | | | |
|---|---|---|---|---|---|---|---|---|---|---|---|
| ANISOU | 2586 | O | HOH | 173 | 6466 | 6618 | 6521 | −185 | −11 | −61 | O |

| | | | | | | | | | | |
|---|---|---|---|---|---|---|---|---|---|---|
| HETATM | 2587 | O | HOH | 174 | 14.550 | 4.591 | 79.369 | 1.00 | 54.16 | O |

| | | | | | | | | | | | |
|---|---|---|---|---|---|---|---|---|---|---|---|
| ANISOU | 2587 | O | HOH | 174 | 6803 | 6773 | 7001 | 24 | −63 | 39 | O |

| | | | | | | | | | | |
|---|---|---|---|---|---|---|---|---|---|---|
| HETATM | 2588 | O | HOH | 175 | 3.502 | 23.110 | 48.771 | 1.00 | 45.77 | O |

| | | | | | | | | | | | |
|---|---|---|---|---|---|---|---|---|---|---|---|
| ANISOU | 2588 | O | HOH | 175 | 5770 | 5943 | 5677 | −13 | −56 | −2 | O |

| | | | | | | | | | | |
|---|---|---|---|---|---|---|---|---|---|---|
| HETATM | 2589 | O | HOH | 176 | 21.028 | 19.774 | 93.260 | 1.00 | 35.01 | O |

| | | | | | | | | | | | |
|---|---|---|---|---|---|---|---|---|---|---|---|
| ANISOU | 2589 | O | HOH | 176 | 4493 | 4631 | 4180 | −114 | 312 | 224 | O |

| | | | | | | | | | | |
|---|---|---|---|---|---|---|---|---|---|---|
| HETATM | 2590 | O | HOH | 177 | 37.317 | 15.925 | 74.887 | 1.00 | 47.41 | O |

| | | | | | | | | | | | |
|---|---|---|---|---|---|---|---|---|---|---|---|
| ANISOU | 2590 | O | HOH | 177 | 6292 | 5883 | 5849 | 146 | −6 | −112 | O |

| | | | | | | | | | | |
|---|---|---|---|---|---|---|---|---|---|---|
| HETATM | 2591 | O | HOH | 178 | 5.970 | 20.098 | 57.147 | 1.00 | 43.65 | O |

| | | | | | | | | | | | |
|---|---|---|---|---|---|---|---|---|---|---|---|
| ANISOU | 2591 | O | HOH | 178 | 5579 | 5670 | 5334 | 112 | −43 | 188 | O |

| | | | | | | | | | | |
|---|---|---|---|---|---|---|---|---|---|---|
| HETATM | 2592 | O | HOH | 180 | 31.762 | 9.526 | 70.915 | 1.00 | 42.55 | O |

| | | | | | | | | | | | |
|---|---|---|---|---|---|---|---|---|---|---|---|
| ANISOU | 2592 | O | HOH | 180 | 5672 | 5064 | 5430 | −172 | 83 | 105 | O |

| | | | | | | | | | | |
|---|---|---|---|---|---|---|---|---|---|---|
| HETATM | 2593 | O | HOH | 181 | 47.822 | 24.554 | 70.761 | 1.00 | 41.72 | O |

| | | | | | | | | | | | |
|---|---|---|---|---|---|---|---|---|---|---|---|
| ANISOU | 2593 | O | HOH | 181 | 5177 | 5233 | 5440 | 136 | 63 | −20 | O |

| | | | | | | | | | | |
|---|---|---|---|---|---|---|---|---|---|---|
| HETATM | 2594 | O | HOH | 182 | 26.734 | 24.919 | 50.172 | 1.00 | 52.79 | O |

| | | | | | | | | | | | |
|---|---|---|---|---|---|---|---|---|---|---|---|
| ANISOU | 2594 | O | HOH | 182 | 6486 | 6848 | 6723 | 28 | 117 | 23 | O |

| | | | | | | | | | | |
|---|---|---|---|---|---|---|---|---|---|---|
| HETATM | 2595 | O | HOH | 183 | 32.925 | 11.685 | 69.753 | 1.00 | 42.43 | O |

| | | | | | | | | | | | |
|---|---|---|---|---|---|---|---|---|---|---|---|
| ANISOU | 2595 | O | HOH | 183 | 5774 | 4874 | 5474 | 65 | −12 | −10 | O |

| | | | | | | | | | | |
|---|---|---|---|---|---|---|---|---|---|---|
| HETATM | 2596 | O | HOH | 184 | 18.971 | 15.202 | 87.889 | 1.00 | 38.16 | O |

| | | | | | | | | | | | |
|---|---|---|---|---|---|---|---|---|---|---|---|
| ANISOU | 2596 | O | HOH | 184 | 4719 | 4617 | 5163 | 37 | 175 | −86 | O |

| | | | | | | | | | | |
|---|---|---|---|---|---|---|---|---|---|---|
| HETATM | 2597 | O | HOH | 185 | 35.218 | 26.224 | 90.859 | 1.00 | 49.60 | O |

| | | | | | | | | | | | |
|---|---|---|---|---|---|---|---|---|---|---|---|
| ANISOU | 2597 | O | HOH | 185 | 6211 | 6225 | 6412 | −62 | 76 | 80 | O |

| | | | | | | | | | | |
|---|---|---|---|---|---|---|---|---|---|---|
| HETATM | 2598 | O | HOH | 186 | 5.227 | 24.542 | 47.195 | 1.00 | 43.45 | O |

| | | | | | | | | | | | |
|---|---|---|---|---|---|---|---|---|---|---|---|
| ANISOU | 2598 | O | HOH | 186 | 5439 | 5733 | 5336 | 59 | 4 | 7 | O |

| | | | | | | | | | | |
|---|---|---|---|---|---|---|---|---|---|---|
| HETATM | 2599 | O | HOH | 187 | 39.009 | 31.723 | 80.027 | 1.00 | 34.64 | O |

| | | | | | | | | | | | |
|---|---|---|---|---|---|---|---|---|---|---|---|
| ANISOU | 2599 | O | HOH | 187 | 4598 | 4576 | 3987 | −233 | −35 | −224 | O |

| | | | | | | | | | | |
|---|---|---|---|---|---|---|---|---|---|---|
| HETATM | 2600 | O | HOH | 188 | 42.214 | 26.523 | 66.397 | 1.00 | 42.66 | O |

| | | | | | | | | | | | |
|---|---|---|---|---|---|---|---|---|---|---|---|
| ANISOU | 2600 | O | HOH | 188 | 5557 | 5491 | 5160 | 179 | 231 | −83 | O |

| | | | | | | | | | | |
|---|---|---|---|---|---|---|---|---|---|---|
| HETATM | 2601 | O | HOH | 189 | 17.465 | 32.770 | 93.454 | 1.00 | 51.95 | O |

| | | | | | | | | | | | |
|---|---|---|---|---|---|---|---|---|---|---|---|
| ANISOU | 2601 | O | HOH | 189 | 6708 | 6623 | 6405 | 16 | 138 | 95 | O |

| | | | | | | | | | | |
|---|---|---|---|---|---|---|---|---|---|---|
| HETATM | 2602 | O | HOH | 190 | 8.735 | 15.447 | 39.420 | 1.00 | 54.50 | O |

| | | | | | | | | | | | |
|---|---|---|---|---|---|---|---|---|---|---|---|
| ANISOU | 2602 | O | HOH | 190 | 6832 | 6858 | 7019 | 0 | −35 | 6 | O |

| | | | | | | | | | | |
|---|---|---|---|---|---|---|---|---|---|---|
| HETATM | 2603 | O | HOH | 191 | −2.607 | 20.239 | 72.368 | 1.00 | 40.07 | O |

| | | | | | | | | | | | |
|---|---|---|---|---|---|---|---|---|---|---|---|
| ANISOU | 2603 | O | HOH | 191 | 4840 | 5063 | 5323 | 14 | 141 | 81 | O |

| | | | | | | | | | | |
|---|---|---|---|---|---|---|---|---|---|---|
| HETATM | 2604 | O | HOH | 193 | 33.794 | 37.511 | 70.614 | 1.00 | 42.65 | O |

| | | | | | | | | | | | |
|---|---|---|---|---|---|---|---|---|---|---|---|
| ANISOU | 2604 | O | HOH | 193 | 5608 | 5288 | 5308 | 145 | −43 | −58 | O |

| | | | | | | | | | | |
|---|---|---|---|---|---|---|---|---|---|---|
| HETATM | 2605 | O | HOH | 195 | 3.507 | 13.927 | 50.971 | 1.00 | 59.90 | O |

| | | | | | | | | | | | |
|---|---|---|---|---|---|---|---|---|---|---|---|
| ANISOU | 2605 | O | HOH | 195 | 7570 | 7678 | 7511 | O | 2 | 8 | O |

| | | | | | | | | | | |
|---|---|---|---|---|---|---|---|---|---|---|
| HETATM | 2606 | O | HOH | 196 | −4.736 | 20.639 | 73.512 | 1.00 | 40.93 | O |

| | | | | | | | | | | | |
|---|---|---|---|---|---|---|---|---|---|---|---|
| ANISOU | 2606 | O | HOH | 196 | 5209 | 4993 | 5352 | −36 | −222 | 85 | O |

| | | | | | | | | | | |
|---|---|---|---|---|---|---|---|---|---|---|
| HETATM | 2607 | O | HOH | 197 | 26.092 | 19.633 | 55.216 | 1.00 | 51.00 | O |

| | | | | | | | | | | | |
|---|---|---|---|---|---|---|---|---|---|---|---|
| ANISOU | 2607 | O | HOH | 197 | 6169 | 6535 | 6675 | 44 | −10 | −122 | O |

| | | | | | | | | | | |
|---|---|---|---|---|---|---|---|---|---|---|
| HETATM | 2608 | O | HOH | 198 | 23.781 | 30.889 | 93.836 | 1.00 | 32.97 | O |

| | | | | | | | | | | | |
|---|---|---|---|---|---|---|---|---|---|---|---|
| ANISOU | 2608 | O | HOH | 198 | 4468 | 4241 | 3820 | −47 | −78 | 114 | O |

| | | | | | | | | | | |
|---|---|---|---|---|---|---|---|---|---|---|
| HETATM | 2609 | O | HOH | 200 | 21.093 | 30.159 | 56.807 | 1.00 | 52.30 | O |

| | | | | | | | | | | | |
|---|---|---|---|---|---|---|---|---|---|---|---|
| ANISOU | 2609 | O | HOH | 200 | 6449 | 6810 | 6614 | 70 | 103 | −34 | O |

| | | | | | | | | | | |
|---|---|---|---|---|---|---|---|---|---|---|
| HETATM | 2610 | O | HOH | 201 | 6.287 | 28.496 | 61.060 | 1.00 | 47.40 | O |

| | | | | | | | | | | | |
|---|---|---|---|---|---|---|---|---|---|---|---|
| ANISOU | 2610 | O | HOH | 201 | 6270 | 5946 | 5795 | 178 | 56 | −66 | O |

| | | | | | | | | | | |
|---|---|---|---|---|---|---|---|---|---|---|
| HETATM | 2611 | O | HOH | 202 | −1.544 | 13.525 | 70.641 | 1.00 | 37.09 | O |

| | | | | | | | | | | | |
|---|---|---|---|---|---|---|---|---|---|---|---|
| ANISOU | 2611 | O | HOH | 202 | 4297 | 4780 | 5016 | −377 | −349 | −40 | O |

| | | | | | | | | | | |
|---|---|---|---|---|---|---|---|---|---|---|
| HETATM | 2612 | O | HOH | 203 | 7.553 | 20.917 | 60.941 | 1.00 | 47.92 | O |

| | | | | | | | | | | | |
|---|---|---|---|---|---|---|---|---|---|---|---|
| ANISOU | 2612 | O | HOH | 203 | 5823 | 6106 | 6279 | −110 | 66 | −11 | O |

| | | | | | | | | | | |
|---|---|---|---|---|---|---|---|---|---|---|
| HETATM | 2613 | O | HOH | 204 | 17.614 | 21.404 | 91.766 | 1.00 | 39.24 | O |

| | | | | | | | | | | | |
|---|---|---|---|---|---|---|---|---|---|---|---|
| ANISOU | 2613 | O | HOH | 204 | 4982 | 4967 | 4958 | −68 | 115 | 139 | O |

| | | | | | | | | | | |
|---|---|---|---|---|---|---|---|---|---|---|
| HETATM | 2614 | O | HOH | 205 | 3.511 | 31.591 | 63.721 | 1.00 | 50.48 | O |

| | | | | | | | | | | | |
|---|---|---|---|---|---|---|---|---|---|---|---|
| ANISOU | 2614 | O | HOH | 205 | 6487 | 6369 | 6325 | 132 | −63 | 48 | O |

| | | | | | | | | | | |
|---|---|---|---|---|---|---|---|---|---|---|
| HETATM | 2615 | O | HOH | 206 | 32.461 | 39.367 | 68.538 | 1.00 | 60.12 | O |

| | | | | | | | | | | | |
|---|---|---|---|---|---|---|---|---|---|---|---|
| ANISOU | 2615 | O | HOH | 206 | 7617 | 7556 | 7670 | 25 | −55 | −82 | O |

| | | | | | | | | | | |
|---|---|---|---|---|---|---|---|---|---|---|
| HETATM | 2616 | O | HOH | 207 | 27.566 | 32.440 | 60.770 | 1.00 | 45.93 | O |

| | | | | | | | | | | | |
|---|---|---|---|---|---|---|---|---|---|---|---|
| ANISOU | 2616 | O | HOH | 207 | 5774 | 5890 | 5788 | −5 | −119 | 49 | O |

| | | | | | | | | | | |
|---|---|---|---|---|---|---|---|---|---|---|
| HETATM | 2617 | O | HOH | 208 | 17.698 | 37.711 | 66.116 | 1.00 | 40.56 | O |

| | | | | | | | | | | | |
|---|---|---|---|---|---|---|---|---|---|---|---|
| ANISOU | 2617 | O | HOH | 208 | 5267 | 4922 | 5224 | 87 | 46 | 245 | O |

| | | | | | | | | | | |
|---|---|---|---|---|---|---|---|---|---|---|
| HETATM | 2618 | O | HOH | 209 | 1.483 | 26.616 | 79.265 | 1.00 | 54.08 | O |

| | | | | | | | | | | | |
|---|---|---|---|---|---|---|---|---|---|---|---|
| ANISOU | 2618 | O | HOH | 209 | 6873 | 6913 | 6760 | −24 | 39 | 66 | O |

| | | | | | | | | | | |
|---|---|---|---|---|---|---|---|---|---|---|
| HETATM | 2619 | O | HOH | 210 | 42.034 | 27.866 | 63.967 | 1.00 | 56.30 | O |

| | | | | | | | | | | | |
|---|---|---|---|---|---|---|---|---|---|---|---|
| ANISOU | 2619 | O | HOH | 210 | 7077 | 7232 | 7083 | 108 | 36 | −14 | O |

| | | | | | | | | | | |
|---|---|---|---|---|---|---|---|---|---|---|
| HETATM | 2620 | O | HOH | 211 | 43.429 | 19.917 | 70.829 | 1.00 | 50.34 | O |

| | | | | | | | | | | | |
|---|---|---|---|---|---|---|---|---|---|---|---|
| ANISOU | 2620 | O | HOH | 211 | 6362 | 6386 | 6381 | 14 | −97 | −26 | O |

| | | | | | | | | | | |
|---|---|---|---|---|---|---|---|---|---|---|
| HETATM | 2621 | O | HOH | 212 | 37.808 | 23.533 | 62.993 | 1.00 | 40.32 | O |

| | | | | | | | | | | | |
|---|---|---|---|---|---|---|---|---|---|---|---|
| ANISOU | 2621 | O | HOH | 212 | 4939 | 5295 | 5086 | −122 | 154 | −65 | O |

| | | | | | | | | | | |
|---|---|---|---|---|---|---|---|---|---|---|
| HETATM | 2622 | O | HOH | 213 | 43.082 | 31.381 | 78.491 | 1.00 | 56.01 | O |

| | | | | | | | | | | | |
|---|---|---|---|---|---|---|---|---|---|---|---|
| ANISOU | 2622 | O | HOH | 213 | 7107 | 7033 | 7141 | −44 | −49 | −85 | O |

| | | | | | | | | | | |
|---|---|---|---|---|---|---|---|---|---|---|
| HETATM | 2623 | O | HOH | 215 | 11.830 | 4.624 | 80.523 | 1.00 | 55.43 | O |

| | | | | | | | | | | | |
|---|---|---|---|---|---|---|---|---|---|---|---|
| ANISOU | 2623 | O | HOH | 215 | 6901 | 6996 | 7162 | −35 | −13 | 76 | O |

| | | | | | | | | | | |
|---|---|---|---|---|---|---|---|---|---|---|
| HETATM | 2624 | O | HOH | 216 | 15.166 | 16.314 | 86.736 | 1.00 | 38.36 | O |

| | | | | | | | | | | | |
|---|---|---|---|---|---|---|---|---|---|---|---|
| ANISOU | 2624 | O | HOH | 216 | 4438 | 5215 | 4921 | −232 | 43 | 30 | O |

| | | | | | | | | | | |
|---|---|---|---|---|---|---|---|---|---|---|
| HETATM | 2625 | O | HOH | 217 | 36.377 | 16.002 | 72.427 | 1.00 | 35.99 | O |

| | | | | | | | | | | | |
|---|---|---|---|---|---|---|---|---|---|---|---|
| ANISOU | 2625 | O | HOH | 217 | 5335 | 3766 | 4575 | −14 | −14 | 33 | O |

| | | | | | | | | | | |
|---|---|---|---|---|---|---|---|---|---|---|
| HETATM | 2626 | O | HOH | 218 | −1.373 | 11.869 | 79.645 | 1.00 | 41.53 | O |

| | | | | | | | | | | | |
|---|---|---|---|---|---|---|---|---|---|---|---|
| ANISOU | 2626 | O | HOH | 218 | 4912 | 5117 | 5751 | −296 | −120 | 20 | O |

| | | | | | | | | | | |
|---|---|---|---|---|---|---|---|---|---|---|
| HETATM | 2627 | O | HOH | 219 | −3.379 | 16.353 | 86.986 | 1.00 | 45.74 | O |

| | | | | | | | | | | | |
|---|---|---|---|---|---|---|---|---|---|---|---|
| ANISOU | 2627 | O | HOH | 219 | 5715 | 5830 | 5833 | 85 | 17 | −43 | O |

| | | | | | | | | | | |
|---|---|---|---|---|---|---|---|---|---|---|
| HETATM | 2628 | O | HOH | 220 | 2.091 | 6.405 | 72.737 | 1.00 | 50.82 | O |

| | | | | | | | | | | | |
|---|---|---|---|---|---|---|---|---|---|---|---|
| ANISOU | 2628 | O | HOH | 220 | 6364 | 6358 | 6589 | −22 | −48 | 13 | O |

| | | | | | | | | | | |
|---|---|---|---|---|---|---|---|---|---|---|
| HETATM | 2629 | O | HOH | 221 | 2.920 | 10.850 | 69.175 | 1.00 | 45.38 | O |

| | | | | | | | | | | | |
|---|---|---|---|---|---|---|---|---|---|---|---|
| ANISOU | 2629 | O | HOH | 221 | 5809 | 5558 | 5877 | −19 | −81 | −71 | O |

| | | | | | | | | | | |
|---|---|---|---|---|---|---|---|---|---|---|
| HETATM | 2630 | O | HOH | 222 | 5.981 | 25.400 | 90.850 | 1.00 | 57.36 | O |

| | | | | | | | | | | | |
|---|---|---|---|---|---|---|---|---|---|---|---|
| ANISOU | 2630 | O | HOH | 222 | 7302 | 7230 | 7264 | 43 | 55 | −27 | O |

| | | | | | | | | | | |
|---|---|---|---|---|---|---|---|---|---|---|
| HETATM | 2631 | O | HOH | 224 | 16.947 | 25.759 | 97.908 | 1.00 | 45.97 | O |

| | | | | | | | | | | | |
|---|---|---|---|---|---|---|---|---|---|---|---|
| ANISOU | 2631 | O | HOH | 224 | 5585 | 6066 | 5815 | 81 | 93 | −5 | O |

| | | | | | | | | | | |
|---|---|---|---|---|---|---|---|---|---|---|
| HETATM | 2632 | O | HOH | 225 | 19.414 | 21.824 | 39.526 | 1.00 | 53.64 | O |

| | | | | | | | | | | | |
|---|---|---|---|---|---|---|---|---|---|---|---|
| ANISOU | 2632 | O | HOH | 225 | 6777 | 6754 | 6848 | −44 | 50 | −87 | O |

| | | | | | | | | | | |
|---|---|---|---|---|---|---|---|---|---|---|
| HETATM | 2633 | O | HOH | 226 | 1.800 | 25.637 | 74.624 | 1.00 | 40.30 | O |

| | | | | | | | | | | | |
|---|---|---|---|---|---|---|---|---|---|---|---|
| ANISOU | 2633 | O | HOH | 226 | 4930 | 5253 | 5128 | −148 | 144 | −70 | O |

| | | | | | | | | | | |
|---|---|---|---|---|---|---|---|---|---|---|
| HETATM | 2634 | O | HOH | 227 | 23.269 | 14.956 | 55.180 | 1.00 | 48.49 | O |

| | | | | | | | | | | | |
|---|---|---|---|---|---|---|---|---|---|---|---|
| ANISOU | 2634 | O | HOH | 227 | 6018 | 6304 | 6101 | 89 | −65 | −63 | O |

| | | | | | | | | | | |
|---|---|---|---|---|---|---|---|---|---|---|
| HETATM | 2635 | O | HOH | 228 | 18.568 | 32.106 | 45.086 | 1.00 | 45.28 | O |

| | | | | | | | | | | | |
|---|---|---|---|---|---|---|---|---|---|---|---|
| ANISOU | 2635 | O | HOH | 228 | 5629 | 5870 | 5706 | 135 | 119 | 94 | O |

| | | | | | | | | | | |
|---|---|---|---|---|---|---|---|---|---|---|
| HETATM | 2636 | O | HOH | 229 | 18.157 | 28.244 | 94.243 | 1.00 | 43.77 | O |

| | | | | | | | | | | | |
|---|---|---|---|---|---|---|---|---|---|---|---|
| ANISOU | 2636 | O | HOH | 229 | 5417 | 5624 | 5591 | 14 | −48 | −195 | O |

| | | | | | | | | | | |
|---|---|---|---|---|---|---|---|---|---|---|
| HETATM | 2637 | O | HOH | 231 | 3.941 | 5.319 | 83.232 | 1.00 | 58.77 | O |

| | | | | | | | | | | | |
|---|---|---|---|---|---|---|---|---|---|---|---|
| ANISOU | 2637 | O | HOH | 231 | 7480 | 7292 | 7557 | −68 | −12 | 33 | O |

| | | | | | | | | | | |
|---|---|---|---|---|---|---|---|---|---|---|
| HETATM | 2638 | O | HOH | 233 | 23.872 | 43.243 | 83.729 | 1.00 | 49.18 | O |

| | | | | | | | | | | | |
|---|---|---|---|---|---|---|---|---|---|---|---|
| ANISOU | 2638 | O | HOH | 233 | 6397 | 6031 | 6259 | −26 | 20 | 45 | O |

| | | | | | | | | | | |
|---|---|---|---|---|---|---|---|---|---|---|
| HETATM | 2639 | O | HOH | 234 | 5.807 | 35.953 | 86.478 | 1.00 | 39.50 | O |

| | | | | | | | | | | | |
|---|---|---|---|---|---|---|---|---|---|---|---|
| ANISOU | 2639 | O | HOH | 234 | 5214 | 4636 | 5158 | 168 | −47 | −181 | O |

| | | | | | | | | | | |
|---|---|---|---|---|---|---|---|---|---|---|
| HETATM | 2640 | O | HOH | 235 | −1.318 | 9.821 | 84.486 | 1.00 | 62.05 | O |

| | | | | | | | | | | | |
|---|---|---|---|---|---|---|---|---|---|---|---|
| ANISOU | 2640 | O | HOH | 235 | 7811 | 7836 | 7927 | −63 | −30 | 13 | O |

| | | | | | | | | | | |
|---|---|---|---|---|---|---|---|---|---|---|
| HETATM | 2641 | O | HOH | 236 | 1.804 | 32.375 | 90.178 | 1.00 | 56.49 | O |

| | | | | | | | | | | | |
|---|---|---|---|---|---|---|---|---|---|---|---|
| ANISOU | 2641 | O | HOH | 236 | 7043 | 7190 | 7232 | −12 | −52 | 5 | O |

| | | | | | | | | | | |
|---|---|---|---|---|---|---|---|---|---|---|
| HETATM | 2642 | O | HOH | 237 | 0.925 | 21.448 | 82.987 | 1.00 | 39.99 | O |

| | | | | | | | | | | | |
|---|---|---|---|---|---|---|---|---|---|---|---|
| ANISOU | 2642 | O | HOH | 237 | 4850 | 5256 | 5088 | −36 | −37 | 75 | O |

| | | | | | | | | | | |
|---|---|---|---|---|---|---|---|---|---|---|
| HETATM | 2643 | O | HOH | 239 | 2.879 | 8.457 | 71.154 | 1.00 | 47.58 | O |

| | | | | | | | | | | | |
|---|---|---|---|---|---|---|---|---|---|---|---|
| ANISOU | 2643 | O | HOH | 239 | 6000 | 5881 | 6197 | −102 | −52 | −53 | O |

| | | | | | | | | | | |
|---|---|---|---|---|---|---|---|---|---|---|
| HETATM | 2644 | O | HOH | 240 | 18.732 | 39.891 | 89.712 | 1.00 | 45.92 | O |

| | | | | | | | | | | | |
|---|---|---|---|---|---|---|---|---|---|---|---|
| ANISOU | 2644 | O | HOH | 240 | 5949 | 5499 | 5997 | 25 | −30 | −26 | O |

| | | | | | | | | | | |
|---|---|---|---|---|---|---|---|---|---|---|
| HETATM | 2645 | O | HOH | 241 | 25.660 | 28.109 | 50.609 | 1.00 | 51.03 | O |

| | | | | | | | | | | | |
|---|---|---|---|---|---|---|---|---|---|---|---|
| ANISOU | 2645 | O | HOH | 241 | 6333 | 6478 | 6578 | −120 | 99 | 67 | O |

| | | | | | | | | | | |
|---|---|---|---|---|---|---|---|---|---|---|
| HETATM | 2646 | O | HOH | 244 | 38.699 | 32.712 | 88.082 | 1.00 | 56.52 | O |

| | | | | | | | | | | | |
|---|---|---|---|---|---|---|---|---|---|---|---|
| ANISOU | 2646 | O | HOH | 244 | 7097 | 7278 | 7100 | −62 | 6 | 26 | O |

| | | | | | | | | | | |
|---|---|---|---|---|---|---|---|---|---|---|
| HETATM | 2647 | O | HOH | 245 | 18.200 | 11.032 | 69.905 | 1.00 | 55.67 | O |

| | | | | | | | | | | | |
|---|---|---|---|---|---|---|---|---|---|---|---|
| ANISOU | 2647 | O | HOH | 245 | 7037 | 7069 | 7046 | 47 | −6 | −62 | O |

| | | | | | | | | | | |
|---|---|---|---|---|---|---|---|---|---|---|
| HETATM | 2648 | O | HOH | 247 | 6.266 | 30.766 | 77.520 | 1.00 | 35.27 | O |

| | | | | | | | | | | | |
|---|---|---|---|---|---|---|---|---|---|---|---|
| ANISOU | 2648 | O | HOH | 247 | 4116 | 4315 | 4970 | 335 | 103 | −7 | O |

| | | | | | | | | | | |
|---|---|---|---|---|---|---|---|---|---|---|
| HETATM | 2649 | O | HOH | 248 | 10.443 | 35.367 | 90.850 | 1.00 | 38.88 | O |

| | | | | | | | | | | | |
|---|---|---|---|---|---|---|---|---|---|---|---|
| ANISOU | 2649 | O | HOH | 248 | 4926 | 4982 | 4866 | −35 | 46 | −99 | O |

| | | | | | | | | | | |
|---|---|---|---|---|---|---|---|---|---|---|
| HETATM | 2650 | O | HOH | 249 | 34.849 | 11.886 | 63.661 | 1.00 | 58.13 | O |

| | | | | | | | | | | | |
|---|---|---|---|---|---|---|---|---|---|---|---|
| ANISOU | 2650 | O | HOH | 249 | 7371 | 7310 | 7407 | 12 | −53 | −1 | O |

| | | | | | | | | | | |
|---|---|---|---|---|---|---|---|---|---|---|
| HETATM | 2651 | O | HOH | 250 | −0.751 | 29.875 | 55.099 | 1.00 | 52.77 | O |

| | | | | | | | | | | | |
|---|---|---|---|---|---|---|---|---|---|---|---|
| ANISOU | 2651 | O | HOH | 250 | 6498 | 6850 | 6703 | −17 | 61 | 16 | O |

| | | | | | | | | | | |
|---|---|---|---|---|---|---|---|---|---|---|
| HETATM | 2652 | O | HOH | 251 | 39.775 | 29.228 | 84.737 | 1.00 | 44.55 | O |

| | | | | | | | | | | | |
|---|---|---|---|---|---|---|---|---|---|---|---|
| ANISOU | 2652 | O | HOH | 251 | 5394 | 5624 | 5909 | −51 | −95 | 5 | O |

| | | | | | | | | | | |
|---|---|---|---|---|---|---|---|---|---|---|
| HETATM | 2653 | O | HOH | 252 | 8.905 | 31.517 | 73.237 | 1.00 | 31.68 | O |

| | | | | | | | | | | | |
|---|---|---|---|---|---|---|---|---|---|---|---|
| ANISOU | 2653 | O | HOH | 252 | 3599 | 4161 | 4276 | −13 | −92 | 199 | O |

| | | | | | | | | | | |
|---|---|---|---|---|---|---|---|---|---|---|
| HETATM | 2654 | O | HOH | 253 | 5.904 | 5.392 | 79.575 | 1.00 | 44.36 | O |

| | | | | | | | | | | | |
|---|---|---|---|---|---|---|---|---|---|---|---|
| ANISOU | 2654 | O | HOH | 253 | 5611 | 5491 | 5754 | −110 | 117 | −17 | O |

| | | | | | | | | | | |
|---|---|---|---|---|---|---|---|---|---|---|
| HETATM | 2655 | O | HOH | 254 | 2.593 | 27.491 | 57.553 | 1.00 | 61.05 | O |

| | | | | | | | | | | | |
|---|---|---|---|---|---|---|---|---|---|---|---|
| ANISOU | 2655 | O | HOH | 254 | 7747 | 7802 | 7645 | 56 | −70 | 19 | O |

| | | | | | | | | | | |
|---|---|---|---|---|---|---|---|---|---|---|
| HETATM | 2656 | O | HOH | 255 | −3.034 | 6.388 | 73.651 | 1.00 | 41.01 | O |

| | | | | | | | | | | | |
|---|---|---|---|---|---|---|---|---|---|---|---|
| ANISOU | 2656 | O | HOH | 255 | 5096 | 5113 | 5373 | −260 | −71 | 37 | O |

| | | | | | | | | | | |
|---|---|---|---|---|---|---|---|---|---|---|
| HETATM | 2657 | O | HOH | 256 | 15.248 | 11.098 | 45.922 | 1.00 | 49.92 | O |

| | | | | | | | | | | | |
|---|---|---|---|---|---|---|---|---|---|---|---|
| ANISOU | 2657 | O | HOH | 256 | 6452 | 6259 | 6256 | −26 | −12 | −3 | O |

| | | | | | | | | | | |
|---|---|---|---|---|---|---|---|---|---|---|
| HETATM | 2658 | O | HOH | 257 | 20.586 | 23.871 | 40.837 | 1.00 | 47.02 | O |

| | | | | | | | | | | | |
|---|---|---|---|---|---|---|---|---|---|---|---|
| ANISOU | 2658 | O | HOH | 257 | 5988 | 6052 | 5825 | −38 | −22 | −76 | O |

| | | | | | | | | | | |
|---|---|---|---|---|---|---|---|---|---|---|
| HETATM | 2659 | O | HOH | 258 | 10.220 | 32.449 | 75.430 | 1.00 | 44.35 | O |

| | | | | | | | | | | | |
|---|---|---|---|---|---|---|---|---|---|---|---|
| ANISOU | 2659 | O | HOH | 258 | 5310 | 5676 | 5867 | −52 | 22 | −83 | O |

| | | | | | | | | | | |
|---|---|---|---|---|---|---|---|---|---|---|
| HETATM | 2660 | O | HOH | 259 | 20.623 | 15.671 | 38.604 | 1.00 | 48.71 | O |

| | | | | | | | | | | | |
|---|---|---|---|---|---|---|---|---|---|---|---|
| ANISOU | 2660 | O | HOH | 259 | 6276 | 6190 | 6043 | −78 | 24 | 29 | O |

| | | | | | | | | | | |
|---|---|---|---|---|---|---|---|---|---|---|
| HETATM | 2661 | O | HOH | 260 | 49.148 | 22.270 | 79.979 | 1.00 | 47.07 | O |

| | | | | | | | | | | | |
|---|---|---|---|---|---|---|---|---|---|---|---|
| ANISOU | 2661 | O | HOH | 260 | 5736 | 6174 | 5974 | 40 | −50 | 112 | O |

| | | | | | | | | | | |
|---|---|---|---|---|---|---|---|---|---|---|
| HETATM | 2662 | O | HOH | 261 | 9.316 | 33.141 | 94.966 | 1.00 | 57.50 | O |

| | | | | | | | | | | | |
|---|---|---|---|---|---|---|---|---|---|---|---|
| ANISOU | 2662 | O | HOH | 261 | 7324 | 7233 | 7291 | 58 | 115 | −26 | O |

| | | | | | | | | | | |
|---|---|---|---|---|---|---|---|---|---|---|
| HETATM | 2663 | O | HOH | 262 | 29.350 | 38.956 | 63.364 | 1.00 | 53.03 | O |

| | | | | | | | | | | | |
|---|---|---|---|---|---|---|---|---|---|---|---|
| ANISOU | 2663 | O | HOH | 262 | 6704 | 6705 | 6739 | −9 | 35 | 82 | O |

| | | | | | | | | | | |
|---|---|---|---|---|---|---|---|---|---|---|
| HETATM | 2664 | O | HOH | 263 | 49.220 | 20.731 | 77.265 | 1.00 | 54.08 | O |

| | | | | | | | | | | | |
|---|---|---|---|---|---|---|---|---|---|---|---|
| ANISOU | 2664 | O | HOH | 263 | 6622 | 6961 | 6967 | 62 | −33 | −45 | O |

| | | | | | | | | | | |
|---|---|---|---|---|---|---|---|---|---|---|
| HETATM | 2665 | O | HOH | 265 | 4.082 | 31.314 | 76.389 | 1.00 | 43.10 | O |

| | | | | | | | | | | | |
|---|---|---|---|---|---|---|---|---|---|---|---|
| ANISOU | 2665 | O | HOH | 265 | 5426 | 5400 | 5548 | 40 | 86 | −68 | O |

| | | | | | | | | | | |
|---|---|---|---|---|---|---|---|---|---|---|
| HETATM | 2666 | O | HOH | 266 | 8.276 | 32.415 | 76.974 | 1.00 | 48.92 | O |

| | | | | | | | | | | | |
|---|---|---|---|---|---|---|---|---|---|---|---|
| ANISOU | 2666 | O | HOH | 266 | 5978 | 6294 | 6317 | −38 | 21 | 23 | O |

| | | | | | | | | | | |
|---|---|---|---|---|---|---|---|---|---|---|
| HETATM | 2667 | O | HOH | 267 | 8.287 | 33.166 | 63.785 | 1.00 | 45.99 | O |

| | | | | | | | | | | | |
|---|---|---|---|---|---|---|---|---|---|---|---|
| ANISOU | 2667 | O | HOH | 267 | 5603 | 6034 | 5836 | 13 | −41 | 21 | O |

| | | | | | | | | | | |
|---|---|---|---|---|---|---|---|---|---|---|
| HETATM | 2668 | O | HOH | 269 | −3.983 | 4.553 | 75.511 | 1.00 | 45.68 | O |

| | | | | | | | | | | | |
|---|---|---|---|---|---|---|---|---|---|---|---|
| ANISOU | 2668 | O | HOH | 269 | 5671 | 5698 | 5986 | −289 | 4 | −5 | O |

| | | | | | | | | | | |
|---|---|---|---|---|---|---|---|---|---|---|
| HETATM | 2669 | O | HOH | 271 | 13.110 | 14.292 | 49.307 | 1.00 | 45.73 | O |

| | | | | | | | | | | | |
|---|---|---|---|---|---|---|---|---|---|---|---|
| ANISOU | 2669 | O | HOH | 271 | 5983 | 5656 | 5736 | −77 | −50 | 33 | O |

| | | | | | | | | | | |
|---|---|---|---|---|---|---|---|---|---|---|
| HETATM | 2670 | O | HOH | 272 | 42.217 | 40.987 | 61.064 | 1.00 | 53.96 | O |

| | | | | | | | | | | | |
|---|---|---|---|---|---|---|---|---|---|---|---|
| ANISOU | 2670 | O | HOH | 272 | 6816 | 6807 | 6881 | −17 | 122 | 51 | O |

| | | | | | | | | | | |
|---|---|---|---|---|---|---|---|---|---|---|
| HETATM | 2671 | O | HOH | 275 | 12.958 | 16.271 | 89.687 | 1.00 | 43.25 | O |

| | | | | | | | | | | | |
|---|---|---|---|---|---|---|---|---|---|---|---|
| ANISOU | 2671 | O | HOH | 275 | 5298 | 5959 | 5177 | −38 | −58 | 56 | O |

| | | | | | | | | | | |
|---|---|---|---|---|---|---|---|---|---|---|
| HETATM | 2672 | O | HOH | 276 | 11.965 | 39.155 | 72.079 | 1.00 | 50.64 | O |

| | | | | | | | | | | | |
|---|---|---|---|---|---|---|---|---|---|---|---|
| ANISOU | 2672 | O | HOH | 276 | 6286 | 6367 | 6587 | 112 | −100 | 64 | O |

| | | | | | | | | | | |
|---|---|---|---|---|---|---|---|---|---|---|
| HETATM | 2673 | O | HOH | 277 | 9.049 | 30.831 | 62.655 | 1.00 | 47.51 | O |

| | | | | | | | | | | | |
|---|---|---|---|---|---|---|---|---|---|---|---|
| ANISOU | 2673 | O | HOH | 277 | 6061 | 6117 | 5874 | 153 | 5 | 102 | O |

| | | | | | | | | | | |
|---|---|---|---|---|---|---|---|---|---|---|
| HETATM | 2674 | O | HOH | 278 | 9.061 | 6.885 | 65.879 | 1.00 | 43.84 | O |

| | | | | | | | | | | | |
|---|---|---|---|---|---|---|---|---|---|---|---|
| ANISOU | 2674 | O | HOH | 278 | 5708 | 5580 | 5369 | 54 | 26 | 18 | O |

| | | | | | | | | | | |
|---|---|---|---|---|---|---|---|---|---|---|
| HETATM | 2675 | O | HOH | 279 | 1.893 | 21.504 | 45.817 | 1.00 | 48.78 | O |

| | | | | | | | | | | | |
|---|---|---|---|---|---|---|---|---|---|---|---|
| ANISOU | 2675 | O | HOH | 279 | 6098 | 6288 | 6147 | 7 | −27 | −3 | O |

| | | | | | | | | | | |
|---|---|---|---|---|---|---|---|---|---|---|
| HETATM | 2676 | O | HOH | 280 | 1.013 | 12.641 | 69.012 | 1.00 | 49.73 | O |

| | | | | | | | | | | | |
|---|---|---|---|---|---|---|---|---|---|---|---|
| ANISOU | 2676 | O | HOH | 280 | 6300 | 6303 | 6293 | −126 | −43 | −107 | O |

| | | | | | | | | | | |
|---|---|---|---|---|---|---|---|---|---|---|
| HETATM | 2677 | O | HOH | 281 | 14.132 | 37.787 | 79.490 | 1.00 | 49.34 | O |

| | | | | | | | | | | | |
|---|---|---|---|---|---|---|---|---|---|---|---|
| ANISOU | 2677 | O | HOH | 281 | 6249 | 6187 | 6310 | −48 | −42 | −24 | O |

| | | | | | | | | | | |
|---|---|---|---|---|---|---|---|---|---|---|
| HETATM | 2678 | O | HOH | 282 | 7.234 | 0.526 | 73.102 | 1.00 | 51.30 | O |

| | | | | | | | | | | | |
|---|---|---|---|---|---|---|---|---|---|---|---|
| ANISOU | 2678 | O | HOH | 282 | 6480 | 6538 | 6475 | −60 | 31 | −25 | O |

| | | | | | | | | | | |
|---|---|---|---|---|---|---|---|---|---|---|
| HETATM | 2679 | O | HOH | 283 | 22.529 | 36.575 | 90.833 | 1.00 | 47.07 | O |

| | | | | | | | | | | | |
|---|---|---|---|---|---|---|---|---|---|---|---|
| ANISOU | 2679 | O | HOH | 283 | 6104 | 5905 | 5876 | 48 | 110 | −60 | O |

| | | | | | | | | | | |
|---|---|---|---|---|---|---|---|---|---|---|
| HETATM | 2680 | O | HOH | 286 | 6.203 | 3.972 | 71.037 | 1.00 | 52.76 | O |

| | | | | | | | | | | | |
|---|---|---|---|---|---|---|---|---|---|---|---|
| ANISOU | 2680 | O | HOH | 286 | 6773 | 6651 | 6623 | −66 | −34 | 43 | O |

| | | | | | | | | | | |
|---|---|---|---|---|---|---|---|---|---|---|
| HETATM | 2681 | O | HOH | 289 | −0.581 | 5.056 | 73.095 | 1.00 | 50.24 | O |

| | | | | | | | | | | | |
|---|---|---|---|---|---|---|---|---|---|---|---|
| ANISOU | 2681 | O | HOH | 289 | 6408 | 6252 | 6429 | −101 | 43 | −4 | O |

| | | | | | | | | | | |
|---|---|---|---|---|---|---|---|---|---|---|
| HETATM | 2682 | O | HOH | 291 | −0.277 | 21.778 | 87.867 | 1.00 | 37.37 | O |

| | | | | | | | | | | | |
|---|---|---|---|---|---|---|---|---|---|---|---|
| ANISOU | 2682 | O | HOH | 291 | 4444 | 4901 | 4852 | 104 | 38 | 69 | O |

| | | | | | | | | | | |
|---|---|---|---|---|---|---|---|---|---|---|
| HETATM | 2683 | O | HOH | 292 | 41.325 | 35.485 | 71.099 | 1.00 | 49.29 | O |

| | | | | | | | | | | | |
|---|---|---|---|---|---|---|---|---|---|---|---|
| ANISOU | 2683 | O | HOH | 292 | 6371 | 6092 | 6266 | −77 | −46 | −19 | O |

| | | | | | | | | | | |
|---|---|---|---|---|---|---|---|---|---|---|
| HETATM | 2684 | O | HOH | 293 | 22.034 | 25.987 | 94.557 | 1.00 | 51.03 | O |

| | | | | | | | | | | | |
|---|---|---|---|---|---|---|---|---|---|---|---|
| ANISOU | 2684 | O | HOH | 293 | 6425 | 6680 | 6286 | 29 | 7 | 63 | O |

| | | | | | | | | | | |
|---|---|---|---|---|---|---|---|---|---|---|
| HETATM | 2685 | O | HOH | 296 | 13.954 | 18.398 | 88.090 | 1.00 | 61.76 | O |

| | | | | | | | | | | | |
|---|---|---|---|---|---|---|---|---|---|---|---|
| ANISOU | 2685 | O | HOH | 296 | 7894 | 7807 | 7766 | −32 | −1 | 7 | O |

| | | | | | | | | | | |
|---|---|---|---|---|---|---|---|---|---|---|
| HETATM | 2686 | O | HOH | 297 | 5.168 | 21.726 | 59.978 | 1.00 | 59.61 | O |

| | | | | | | | | | | | |
|---|---|---|---|---|---|---|---|---|---|---|---|
| ANISOU | 2686 | O | HOH | 297 | 7643 | 7504 | 7501 | 22 | −82 | 51 | O |

| | | | | | | | | | | |
|---|---|---|---|---|---|---|---|---|---|---|
| HETATM | 2687 | O | HOH | 298 | 26.854 | 21.908 | 50.588 | 1.00 | 61.24 | O |

| | | | | | | | | | | | |
|---|---|---|---|---|---|---|---|---|---|---|---|
| ANISOU | 2687 | O | HOH | 298 | 7736 | 7772 | 7759 | 80 | 49 | −68 | O |

| | | | | | | | | | | |
|---|---|---|---|---|---|---|---|---|---|---|
| HETATM | 2688 | O | HOH | 299 | 27.672 | 38.514 | 93.272 | 1.00 | 49.59 | O |

| | | | | | | | | | | | |
|---|---|---|---|---|---|---|---|---|---|---|---|
| ANISOU | 2688 | O | HOH | 299 | 6503 | 6079 | 6259 | 5 | 4 | −47 | O |

| | | | | | | | | | | |
|---|---|---|---|---|---|---|---|---|---|---|
| HETATM | 2689 | O | HOH | 301 | 21.230 | 36.993 | 82.820 | 1.00 | 47.70 | O |

| | | | | | | | | | | | |
|---|---|---|---|---|---|---|---|---|---|---|---|
| ANISOU | 2689 | O | HOH | 301 | 6164 | 5938 | 6023 | 54 | 134 | −6 | O |

| | | | | | | | | | | |
|---|---|---|---|---|---|---|---|---|---|---|
| HETATM | 2690 | O | HOH | 302 | 15.478 | 14.274 | 42.122 | 1.00 | 43.47 | O |

| | | | | | | | | | | | |
|---|---|---|---|---|---|---|---|---|---|---|---|
| ANISOU | 2690 | O | HOH | 302 | 5474 | 5495 | 5548 | 66 | 18 | −21 | O |

| | | | | | | | | | | |
|---|---|---|---|---|---|---|---|---|---|---|
| HETATM | 2691 | O | HOH | 303 | −5.338 | 15.977 | 71.954 | 1.00 | 60.72 | O |

| | | | | | | | | | | | |
|---|---|---|---|---|---|---|---|---|---|---|---|
| ANISOU | 2691 | O | HOH | 303 | 7619 | 7722 | 7728 | −3 | −6 | 49 | O |

| | | | | | | | | | | |
|---|---|---|---|---|---|---|---|---|---|---|
| HETATM | 2692 | O | HOH | 304 | 42.752 | 16.703 | 75.054 | 1.00 | 48.53 | O |

| | | | | | | | | | | | |
|---|---|---|---|---|---|---|---|---|---|---|---|
| ANISOU | 2692 | O | HOH | 304 | 6214 | 6081 | 6144 | 106 | −20 | −78 | O |

| | | | | | | | | | | |
|---|---|---|---|---|---|---|---|---|---|---|
| HETATM | 2693 | O | HOH | 305 | 23.884 | 14.130 | 69.347 | 1.00 | 48.88 | O |

| | | | | | | | | | | | |
|---|---|---|---|---|---|---|---|---|---|---|---|
| ANISOU | 2693 | O | HOH | 305 | 6421 | 5890 | 6261 | 43 | −144 | −18 | O |

| | | | | | | | | | | |
|---|---|---|---|---|---|---|---|---|---|---|
| HETATM | 2694 | O | HOH | 306 | 12.506 | 36.009 | 89.034 | 1.00 | 47.42 | O |

| | | | | | | | | | | | |
|---|---|---|---|---|---|---|---|---|---|---|---|
| ANISOU | 2694 | O | HOH | 306 | 5895 | 5874 | 6249 | −27 | 41 | −55 | O |

| | | | | | | | | | | |
|---|---|---|---|---|---|---|---|---|---|---|
| HETATM | 2695 | O | HOH | 307 | 25.553 | 43.200 | 77.760 | 1.00 | 57.49 | O |

| | | | | | | | | | | | |
|---|---|---|---|---|---|---|---|---|---|---|---|
| ANISOU | 2695 | O | HOH | 307 | 7410 | 7119 | 7314 | 63 | 91 | 7 | O |

| | | | | | | | | | | |
|---|---|---|---|---|---|---|---|---|---|---|
| HETATM | 2696 | O | HOH | 308 | 25.024 | 31.642 | 61.021 | 1.00 | 47.12 | O |

| | | | | | | | | | | | |
|---|---|---|---|---|---|---|---|---|---|---|---|
| ANISOU | 2696 | O | HOH | 308 | 5903 | 6069 | 5933 | 67 | 50 | 51 | O |

| | | | | | | | | | | |
|---|---|---|---|---|---|---|---|---|---|---|
| HETATM | 2697 | O | HOH | 309 | 18.987 | 13.289 | 90.654 | 1.00 | 56.32 | O |

| | | | | | | | | | | | |
|---|---|---|---|---|---|---|---|---|---|---|---|
| ANISOU | 2697 | O | HOH | 309 | 7081 | 7156 | 7162 | −43 | 33 | −52 | O |

| | | | | | | | | | | |
|---|---|---|---|---|---|---|---|---|---|---|
| HETATM | 2698 | O | HOH | 311 | 12.450 | 34.857 | 60.508 | 1.00 | 47.50 | O |

| | | | | | | | | | | | |
|---|---|---|---|---|---|---|---|---|---|---|---|
| ANISOU | 2698 | O | HOH | 311 | 6100 | 6005 | 5941 | −22 | −64 | −27 | O |

| | | | | | | | | | | |
|---|---|---|---|---|---|---|---|---|---|---|
| HETATM | 2699 | O | HOH | 313 | 6.929 | 37.243 | 50.936 | 1.00 | 53.63 | O |

| | | | | | | | | | | | |
|---|---|---|---|---|---|---|---|---|---|---|---|
| ANISOU | 2699 | O | HOH | 313 | 6873 | 6781 | 6723 | −7 | 5 | 50 | O |

| | | | | | | | | | | |
|---|---|---|---|---|---|---|---|---|---|---|
| HETATM | 2700 | O | HOH | 314 | 33.831 | 42.307 | 68.854 | 1.00 | 53.16 | O |

| | | | | | | | | | | | |
|---|---|---|---|---|---|---|---|---|---|---|---|
| ANISOU | 2700 | O | HOH | 314 | 6806 | 6825 | 6567 | 92 | 7 | −31 | O |

| | | | | | | | | | | |
|---|---|---|---|---|---|---|---|---|---|---|
| HETATM | 2701 | O | HOH | 315 | 34.967 | 27.827 | 55.837 | 1.00 | 56.21 | O |

| | | | | | | | | | | | |
|---|---|---|---|---|---|---|---|---|---|---|---|
| ANISOU | 2701 | O | HOH | 315 | 7214 | 7170 | 6972 | −42 | 114 | −22 | O |

| | | | | | | | | | | |
|---|---|---|---|---|---|---|---|---|---|---|
| HETATM | 2702 | O | HOH | 317 | 19.424 | 25.271 | 42.768 | 1.00 | 42.58 | O |

| | | | | | | | | | | | |
|---|---|---|---|---|---|---|---|---|---|---|---|
| ANISOU | 2702 | O | HOH | 317 | 5366 | 5677 | 5137 | 74 | −53 | 53 | O |

| | | | | | | | | | | |
|---|---|---|---|---|---|---|---|---|---|---|
| HETATM | 2703 | O | HOH | 318 | 40.816 | 32.777 | 78.372 | 1.00 | 46.80 | O |

| | | | | | | | | | | | |
|---|---|---|---|---|---|---|---|---|---|---|---|
| ANISOU | 2703 | O | HOH | 318 | 6113 | 5558 | 6113 | −105 | 7 | −5 | O |

| | | | | | | | | | | |
|---|---|---|---|---|---|---|---|---|---|---|
| HETATM | 2704 | O | HOH | 319 | 2.161 | 14.590 | 46.878 | 1.00 | 59.54 | O |

| | | | | | | | | | | | |
|---|---|---|---|---|---|---|---|---|---|---|---|
| ANISOU | 2704 | O | HOH | 319 | 7579 | 7458 | 7584 | −25 | −34 | −65 | O |

| | | | | | | | | | | |
|---|---|---|---|---|---|---|---|---|---|---|
| HETATM | 2705 | O | HOH | 321 | 3.078 | 17.075 | 42.925 | 1.00 | 62.62 | O |

| | | | | | | | | | | | |
|---|---|---|---|---|---|---|---|---|---|---|---|
| ANISOU | 2705 | O | HOH | 321 | 7933 | 7873 | 7985 | −12 | 38 | −32 | O |

| | | | | | | | | | | |
|---|---|---|---|---|---|---|---|---|---|---|
| HETATM | 2706 | O | HOH | 322 | 4.695 | 27.583 | 93.438 | 1.00 | 67.19 | O |

| | | | | | | | | | | | |
|---|---|---|---|---|---|---|---|---|---|---|---|
| ANISOU | 2706 | O | HOH | 322 | 8512 | 8561 | 8457 | −20 | 23 | 17 | O |

| | | | | | | | | | | |
|---|---|---|---|---|---|---|---|---|---|---|
| HETATM | 2707 | O | HOH | 323 | 6.865 | 14.509 | 55.845 | 1.00 | 56.89 | O |

| | | | | | | | | | | | |
|---|---|---|---|---|---|---|---|---|---|---|---|
| ANISOU | 2707 | O | HOH | 323 | 7303 | 7201 | 7110 | −18 | 3 | −10 | O |

| | | | | | | | | | | |
|---|---|---|---|---|---|---|---|---|---|---|
| HETATM | 2708 | O | HOH | 324 | 15.415 | 4.434 | 85.045 | 1.00 | 55.66 | O |

| | | | | | | | | | | | |
|---|---|---|---|---|---|---|---|---|---|---|---|
| ANISOU | 2708 | O | HOH | 324 | 7140 | 7025 | 6984 | 63 | −43 | 21 | O |

| | | | | | | | | | | |
|---|---|---|---|---|---|---|---|---|---|---|
| HETATM | 2709 | O | HOH | 326 | 16.129 | 6.582 | 72.587 | 1.00 | 49.49 | O |

| | | | | | | | | | | | |
|---|---|---|---|---|---|---|---|---|---|---|---|
| ANISOU | 2709 | O | HOH | 326 | 6188 | 6233 | 6383 | 168 | 0 | −78 | O |

| | | | | | | | | | | |
|---|---|---|---|---|---|---|---|---|---|---|
| HETATM | 2710 | O | HOH | 327 | 33.441 | 36.723 | 62.911 | 1.00 | 55.80 | O |

| | | | | | | | | | | | |
|---|---|---|---|---|---|---|---|---|---|---|---|
| ANISOU | 2710 | O | HOH | 327 | 7211 | 7012 | 6980 | −43 | 8 | 61 | O |

| | | | | | | | | | | |
|---|---|---|---|---|---|---|---|---|---|---|
| HETATM | 2711 | O | HOH | 330 | −0.292 | 24.854 | 78.344 | 1.00 | 56.12 | O |

| | | | | | | | | | | | |
|---|---|---|---|---|---|---|---|---|---|---|---|
| ANISOU | 2711 | O | HOH | 330 | 7181 | 7159 | 6983 | 58 | −61 | 63 | O |

| | | | | | | | | | | |
|---|---|---|---|---|---|---|---|---|---|---|
| HETATM | 2712 | O | HOH | 331 | −2.262 | 16.030 | 67.431 | 1.00 | 43.84 | O |

| | | | | | | | | | | | |
|---|---|---|---|---|---|---|---|---|---|---|---|
| ANISOU | 2712 | O | HOH | 331 | 5600 | 5401 | 5657 | −48 | −107 | −143 | O |

| | | | | | | | | | | |
|---|---|---|---|---|---|---|---|---|---|---|
| HETATM | 2713 | O | HOH | 332 | 3.329 | 33.043 | 78.532 | 1.00 | 49.89 | O |

| | | | | | | | | | | | |
|---|---|---|---|---|---|---|---|---|---|---|---|
| ANISOU | 2713 | O | HOH | 332 | 6511 | 6227 | 6216 | 58 | 99 | −24 | O |

| | | | | | | | | | | |
|---|---|---|---|---|---|---|---|---|---|---|
| HETATM | 2714 | O | HOH | 335 | 15.236 | 11.644 | 43.279 | 1.00 | 45.72 | O |

| | | | | | | | | | | | |
|---|---|---|---|---|---|---|---|---|---|---|---|
| ANISOU | 2714 | O | HOH | 335 | 5693 | 5772 | 5905 | 95 | 33 | −36 | O |

| | | | | | | | | | | |
|---|---|---|---|---|---|---|---|---|---|---|
| HETATM | 2715 | O | HOH | 336 | 5.226 | 24.476 | 44.257 | 1.00 | 46.11 | O |

| | | | | | | | | | | | |
|---|---|---|---|---|---|---|---|---|---|---|---|
| ANISOU | 2715 | O | HOH | 336 | 5835 | 6003 | 5682 | −2 | 47 | 52 | O |

| | | | | | | | | | | |
|---|---|---|---|---|---|---|---|---|---|---|
| HETATM | 2716 | O | HOH | 338 | 37.716 | 19.152 | 63.872 | 1.00 | 47.85 | O |

| | | | | | | | | | | | |
|---|---|---|---|---|---|---|---|---|---|---|---|
| ANISOU | 2716 | O | HOH | 338 | 6149 | 5815 | 6217 | −10 | 83 | −36 | O |

| | | | | | | | | | | |
|---|---|---|---|---|---|---|---|---|---|---|
| HETATM | 2717 | O | HOH | 340 | −0.279 | 14.811 | 65.962 | 1.00 | 55.82 | O |

| | | | | | | | | | | | |
|---|---|---|---|---|---|---|---|---|---|---|---|
| ANISOU | 2717 | O | HOH | 340 | 7087 | 7100 | 7020 | 49 | −30 | −30 | O |

| | | | | | | | | | | |
|---|---|---|---|---|---|---|---|---|---|---|
| HETATM | 2718 | O | HOH | 341 | 17.272 | 10.150 | 42.135 | 1.00 | 46.09 | O |

| | | | | | | | | | | | |
|---|---|---|---|---|---|---|---|---|---|---|---|
| ANISOU | 2718 | O | HOH | 341 | 5845 | 5850 | 5817 | −109 | −6 | 3 | O |

| | | | | | | | | | | |
|---|---|---|---|---|---|---|---|---|---|---|
| HETATM | 2719 | O | HOH | 342 | 33.731 | 21.964 | 59.151 | 1.00 | 48.16 | O |

| | | | | | | | | | | | |
|---|---|---|---|---|---|---|---|---|---|---|---|
| ANISOU | 2719 | O | HOH | 342 | 6050 | 6261 | 5987 | −82 | 99 | −143 | O |

| | | | | | | | | | | |
|---|---|---|---|---|---|---|---|---|---|---|
| HETATM | 2720 | O | HOH | 343 | 3.315 | 13.263 | 67.070 | 1.00 | 54.80 | O |

| | | | | | | | | | | | |
|---|---|---|---|---|---|---|---|---|---|---|---|
| ANISOU | 2720 | O | HOH | 343 | 6862 | 6874 | 7087 | −87 | 87 | −19 | O |

| | | | | | | | | | | |
|---|---|---|---|---|---|---|---|---|---|---|
| HETATM | 2721 | O | HOH | 344 | 9.552 | 29.955 | 43.445 | 1.00 | 47.43 | O |

| | | | | | | | | | | | |
|---|---|---|---|---|---|---|---|---|---|---|---|
| ANISOU | 2721 | O | HOH | 344 | 6003 | 5977 | 6040 | 39 | −96 | −3 | O |

| | | | | | | | | | | |
|---|---|---|---|---|---|---|---|---|---|---|
| HETATM | 2722 | O | HOH | 345 | 31.518 | 37.945 | 65.887 | 1.00 | 47.47 | O |

| | | | | | | | | | | | |
|---|---|---|---|---|---|---|---|---|---|---|---|
| ANISOU | 2722 | O | HOH | 345 | 6008 | 5995 | 6035 | −80 | 135 | 28 | O |

| | | | | | | | | | | |
|---|---|---|---|---|---|---|---|---|---|---|
| HETATM | 2723 | O | HOH | 347 | 11.233 | 7.838 | 64.305 | 1.00 | 53.52 | O |

| | | | | | | | | | | | |
|---|---|---|---|---|---|---|---|---|---|---|---|
| ANISOU | 2723 | O | HOH | 347 | 6849 | 6681 | 6806 | 53 | −13 | −110 | O |

| | | | | | | | | | | |
|---|---|---|---|---|---|---|---|---|---|---|
| HETATM | 2724 | O | HOH | 350 | 13.328 | 38.241 | 87.887 | 1.00 | 47.64 | O |

| | | | | | | | | | | | |
|---|---|---|---|---|---|---|---|---|---|---|---|
| ANISOU | 2724 | O | HOH | 350 | 5962 | 5927 | 6213 | −116 | 46 | −19 | O |

| | | | | | | | | | | |
|---|---|---|---|---|---|---|---|---|---|---|
| HETATM | 2725 | O | HOH | 351 | 25.523 | 36.758 | 93.170 | 1.00 | 60.03 | O |

| | | | | | | | | | | | |
|---|---|---|---|---|---|---|---|---|---|---|---|
| ANISOU | 2725 | O | HOH | 351 | 7656 | 7563 | 7589 | −5 | 13 | 29 | O |

| | | | | | | | | | | |
|---|---|---|---|---|---|---|---|---|---|---|
| HETATM | 2726 | O | HOH | 352 | 26.921 | 40.234 | 91.104 | 1.00 | 51.06 | O |

| | | | | | | | | | | | |
|---|---|---|---|---|---|---|---|---|---|---|---|
| ANISOU | 2726 | O | HOH | 352 | 6818 | 5989 | 6592 | −117 | −54 | 15 | O |

| | | | | | | | | | | |
|---|---|---|---|---|---|---|---|---|---|---|
| HETATM | 2727 | O | HOH | 353 | 13.055 | 2.690 | 69.900 | 1.00 | 62.11 | O |

| | | | | | | | | | | | |
|---|---|---|---|---|---|---|---|---|---|---|---|
| ANISOU | 2727 | O | HOH | 353 | 7940 | 7809 | 7849 | 21 | −46 | −47 | O |

| | | | | | | | | | | |
|---|---|---|---|---|---|---|---|---|---|---|
| HETATM | 2728 | O | HOH | 355 | 2.495 | 34.790 | 83.626 | 1.00 | 48.14 | O |

| | | | | | | | | | | | |
|---|---|---|---|---|---|---|---|---|---|---|---|
| ANISOU | 2728 | O | HOH | 355 | 6003 | 5987 | 6301 | 77 | −181 | 11 | O |

| | | | | | | | | | | |
|---|---|---|---|---|---|---|---|---|---|---|
| HETATM | 2729 | O | HOH | 357 | 8.408 | 13.997 | 61.499 | 1.00 | 47.34 | O |

| | | | | | | | | | | | |
|---|---|---|---|---|---|---|---|---|---|---|---|
| ANISOU | 2729 | O | HOH | 357 | 6049 | 5853 | 6087 | −82 | 27 | −128 | O |

| | | | | | | | | | | |
|---|---|---|---|---|---|---|---|---|---|---|
| HETATM | 2730 | O | HOH | 358 | 17.978 | 9.188 | 71.905 | 1.00 | 59.37 | O |

| | | | | | | | | | | | |
|---|---|---|---|---|---|---|---|---|---|---|---|
| ANISOU | 2730 | O | HOH | 358 | 7436 | 7550 | 7574 | 47 | −55 | −21 | O |

| | | | | | | | | | | |
|---|---|---|---|---|---|---|---|---|---|---|
| HETATM | 2731 | O | HOH | 359 | 25.220 | 24.855 | 47.581 | 1.00 | 56.34 | O |

| | | | | | | | | | | | |
|---|---|---|---|---|---|---|---|---|---|---|---|
| ANISOU | 2731 | O | HOH | 359 | 7003 | 7178 | 7226 | −35 | 49 | 48 | O |

| | | | | | | | | | | |
|---|---|---|---|---|---|---|---|---|---|---|
| HETATM | 2732 | O | HOH | 360 | 8.365 | 39.797 | 52.554 | 1.00 | 57.70 | O |

| | | | | | | | | | | | |
|---|---|---|---|---|---|---|---|---|---|---|---|
| ANISOU | 2732 | O | HOH | 360 | 7369 | 7257 | 7299 | 24 | 14 | 30 | O |

| | | | | | | | | | | |
|---|---|---|---|---|---|---|---|---|---|---|
| HETATM | 2733 | O | HOH | 362 | 8.453 | 6.194 | 85.366 | 1.00 | 43.29 | O |

| | | | | | | | | | | | |
|---|---|---|---|---|---|---|---|---|---|---|---|
| ANISOU | 2733 | O | HOH | 362 | 5448 | 5321 | 5681 | −170 | 22 | 39 | O |

| | | | | | | | | | | |
|---|---|---|---|---|---|---|---|---|---|---|
| HETATM | 2734 | O | HOH | 364 | 3.892 | 23.307 | 62.053 | 1.00 | 52.89 | O |

| | | | | | | | | | | | |
|---|---|---|---|---|---|---|---|---|---|---|---|
| ANISOU | 2734 | O | HOH | 364 | 6740 | 6637 | 6718 | 13 | −39 | −1 | O |

| | | | | | | | | | | |
|---|---|---|---|---|---|---|---|---|---|---|
| HETATM | 2735 | O | HOH | 365 | 22.092 | 41.480 | 85.519 | 1.00 | 59.49 | O |

| | | | | | | | | | | | |
|---|---|---|---|---|---|---|---|---|---|---|---|
| ANISOU | 2735 | O | HOH | 365 | 7576 | 7466 | 7560 | −20 | −9 | −41 | O |

| | | | | | | | | | | |
|---|---|---|---|---|---|---|---|---|---|---|
| HETATM | 2736 | O | HOH | 366 | 14.864 | 41.955 | 74.200 | 1.00 | 52.35 | O |

| | | | | | | | | | | | |
|---|---|---|---|---|---|---|---|---|---|---|---|
| ANISOU | 2736 | O | HOH | 366 | 6698 | 6451 | 6742 | 35 | 3 | 6 | O |

| | | | | | | | | | | |
|---|---|---|---|---|---|---|---|---|---|---|
| HETATM | 2737 | O | HOH | 367 | 3.269 | 23.566 | 51.675 | 1.00 | 54.55 | O |

| | | | | | | | | | | | |
|---|---|---|---|---|---|---|---|---|---|---|---|
| ANISOU | 2737 | O | HOH | 367 | 6765 | 6914 | 7048 | 39 | −82 | −15 | O |

| | | | | | | | | | | |
|---|---|---|---|---|---|---|---|---|---|---|
| HETATM | 2738 | O | HOH | 369 | 7.946 | 3.294 | 79.018 | 1.00 | 48.66 | O |

| | | | | | | | | | | | |
|---|---|---|---|---|---|---|---|---|---|---|---|
| ANISOU | 2738 | O | HOH | 369 | 6277 | 5968 | 6242 | −38 | 46 | 23 | O |

| | | | | | | | | | | |
|---|---|---|---|---|---|---|---|---|---|---|
| HETATM | 2739 | O | HOH | 371 | −5.312 | 7.469 | 77.838 | 1.00 | 56.64 | O |

| | | | | | | | | | | | |
|---|---|---|---|---|---|---|---|---|---|---|---|
| ANISOU | 2739 | O | HOH | 371 | 7159 | 7120 | 7242 | −88 | −16 | 44 | O |

| | | | | | | | | | | |
|---|---|---|---|---|---|---|---|---|---|---|
| HETATM | 2740 | O | HOH | 372 | 12.502 | 34.453 | 69.116 | 1.00 | 50.93 | O |

| | | | | | | | | | | | |
|---|---|---|---|---|---|---|---|---|---|---|---|
| ANISOU | 2740 | O | HOH | 372 | 6395 | 6446 | 6509 | 39 | −96 | −23 | O |

| | | | | | | | | | | |
|---|---|---|---|---|---|---|---|---|---|---|
| HETATM | 2741 | O | HOH | 373 | 23.748 | 29.185 | 56.362 | 1.00 | 51.90 | O |

| | | | | | | | | | | | |
|---|---|---|---|---|---|---|---|---|---|---|---|
| ANISOU | 2741 | O | HOH | 373 | 6496 | 6645 | 6580 | 42 | 77 | 20 | O |

| | | | | | | | | | | |
|---|---|---|---|---|---|---|---|---|---|---|
| HETATM | 2742 | O | HOH | 374 | 25.326 | 36.263 | 68.141 | 1.00 | 45.43 | O |

| | | | | | | | | | | | |
|---|---|---|---|---|---|---|---|---|---|---|---|
| ANISOU | 2742 | O | HOH | 374 | 5757 | 5923 | 5580 | 50 | 35 | 159 | O |

| | | | | | | | | | | |
|---|---|---|---|---|---|---|---|---|---|---|
| HETATM | 2743 | O | HOH | 375 | 4.034 | 36.060 | 89.188 | 1.00 | 52.68 | O |

| | | | | | | | | | | | |
|---|---|---|---|---|---|---|---|---|---|---|---|
| ANISOU | 2743 | O | HOH | 375 | 6664 | 6524 | 6830 | 108 | 32 | −119 | O |

| | | | | | | | | | | |
|---|---|---|---|---|---|---|---|---|---|---|
| HETATM | 2744 | O | HOH | 376 | 8.774 | 37.413 | 89.385 | 1.00 | 58.93 | O |

| | | | | | | | | | | | |
|---|---|---|---|---|---|---|---|---|---|---|---|
| ANISOU | 2744 | O | HOH | 376 | 7501 | 7465 | 7425 | −42 | 34 | −78 | O |

| | | | | | | | | | | |
|---|---|---|---|---|---|---|---|---|---|---|
| HETATM | 2745 | O | HOH | 377 | 39.698 | 40.850 | 59.792 | 1.00 | 56.29 | O |

| | | | | | | | | | | | |
|---|---|---|---|---|---|---|---|---|---|---|---|
| ANISOU | 2745 | O | HOH | 377 | 7140 | 7108 | 7139 | −63 | 16 | −48 | O |

| | | | | | | | | | | |
|---|---|---|---|---|---|---|---|---|---|---|
| HETATM | 2746 | O | HOH | 378 | 13.215 | 24.355 | 40.367 | 1.00 | 49.52 | O |

| | | | | | | | | | | | |
|---|---|---|---|---|---|---|---|---|---|---|---|
| ANISOU | 2746 | O | HOH | 378 | 6422 | 6371 | 6024 | −84 | 96 | 147 | O |

| | | | | | | | | | | |
|---|---|---|---|---|---|---|---|---|---|---|
| HETATM | 2747 | O | HOH | 380 | 20.951 | 39.755 | 83.302 | 1.00 | 54.05 | O |

| | | | | | | | | | | | |
|---|---|---|---|---|---|---|---|---|---|---|---|
| ANISOU | 2747 | O | HOH | 380 | 6742 | 6918 | 6877 | 8 | −16 | 36 | O |

| | | | | | | | | | | |
|---|---|---|---|---|---|---|---|---|---|---|
| HETATM | 2748 | O | HOH | 381 | 16.135 | 12.472 | 61.601 | 1.00 | 56.44 | O |

| | | | | | | | | | | | |
|---|---|---|---|---|---|---|---|---|---|---|---|
| ANISOU | 2748 | O | HOH | 381 | 7190 | 7125 | 7131 | 7 | 55 | −11 | O |

| | | | | | | | | | | |
|---|---|---|---|---|---|---|---|---|---|---|
| HETATM | 2749 | O | HOH | 382 | 21.722 | 14.220 | 67.972 | 1.00 | 63.83 | O |

| | | | | | | | | | | | |
|---|---|---|---|---|---|---|---|---|---|---|---|
| ANISOU | 2749 | O | HOH | 382 | 8090 | 8138 | 8024 | −41 | −10 | 16 | O |

| | | | | | | | | | | |
|---|---|---|---|---|---|---|---|---|---|---|
| HETATM | 2750 | O | HOH | 384 | 6.011 | 11.185 | 67.542 | 1.00 | 57.41 | O |

| | | | | | | | | | | | |
|---|---|---|---|---|---|---|---|---|---|---|---|
| ANISOU | 2750 | O | HOH | 384 | 7324 | 7183 | 7305 | 45 | −5 | 86 | O |

| | | | | | | | | | | |
|---|---|---|---|---|---|---|---|---|---|---|
| HETATM | 2751 | O | HOH | 385 | 17.439 | 40.675 | 87.096 | 1.00 | 58.27 | O |

| | | | | | | | | | | | |
|---|---|---|---|---|---|---|---|---|---|---|---|
| ANISOU | 2751 | O | HOH | 385 | 7270 | 7407 | 7463 | 107 | 29 | −36 | O |

| | | | | | | | | | | |
|---|---|---|---|---|---|---|---|---|---|---|
| HETATM | 2752 | O | HOH | 386 | 45.716 | 33.327 | 69.519 | 1.00 | 49.83 | O |

| | | | | | | | | | | | |
|---|---|---|---|---|---|---|---|---|---|---|---|
| ANISOU | 2752 | O | HOH | 386 | 6362 | 6243 | 6329 | −120 | 2 | −24 | O |

| | | | | | | | | | | |
|---|---|---|---|---|---|---|---|---|---|---|
| HETATM | 2753 | O | HOH | 394 | 14.879 | 4.605 | 70.173 | 1.00 | 55.59 | O |

| | | | | | | | | | | | |
|---|---|---|---|---|---|---|---|---|---|---|---|
| ANISOU | 2753 | O | HOH | 394 | 7098 | 6966 | 7058 | 28 | −55 | 68 | O |

| | | | | | | | | | | |
|---|---|---|---|---|---|---|---|---|---|---|
| HETATM | 2754 | O | HOH | 396 | 9.997 | 11.781 | 91.683 | 1.00 | 57.00 | O |

| | | | | | | | | | | | |
|---|---|---|---|---|---|---|---|---|---|---|---|
| ANISOU | 2754 | O | HOH | 396 | 6965 | 7380 | 7314 | −10 | −7 | −10 | O |

| | | | | | | | | | | |
|---|---|---|---|---|---|---|---|---|---|---|
| HETATM | 2755 | O | HOH | 398 | 0.496 | 16.727 | 50.862 | 1.00 | 58.43 | O |

| | | | | | | | | | | | |
|---|---|---|---|---|---|---|---|---|---|---|---|
| ANISOU | 2755 | O | HOH | 398 | 7313 | 7415 | 7474 | −20 | −5 | −24 | O |

| | | | | | | | | | | |
|---|---|---|---|---|---|---|---|---|---|---|
| HETATM | 2756 | O | HOH | 399 | 2.187 | 20.776 | 54.264 | 1.00 | 51.45 | O |

| | | | | | | | | | | | |
|---|---|---|---|---|---|---|---|---|---|---|---|
| ANISOU | 2756 | O | HOH | 399 | 6482 | 6690 | 6376 | −10 | 91 | −100 | O |

| | | | | | | | | | | |
|---|---|---|---|---|---|---|---|---|---|---|
| HETATM | 2757 | O | HOH | 400 | 31.245 | 20.318 | 56.035 | 1.00 | 54.12 | O |

| | | | | | | | | | | | |
|---|---|---|---|---|---|---|---|---|---|---|---|
| ANISOU | 2757 | O | HOH | 400 | 6761 | 7015 | 6787 | −19 | −21 | −87 | O |

| | | | | | | | | | | |
|---|---|---|---|---|---|---|---|---|---|---|
| HETATM | 2758 | O | HOH | 401 | 3.637 | 13.527 | 60.726 | 1.00 | 64.33 | O |

| | | | | | | | | | | | |
|---|---|---|---|---|---|---|---|---|---|---|---|
| ANISOU | 2758 | O | HOH | 401 | 8139 | 8215 | 8087 | −60 | −19 | −6 | O |

| | | | | | | | | | | |
|---|---|---|---|---|---|---|---|---|---|---|
| HETATM | 2759 | O | HOH | 403 | 3.546 | 4.378 | 70.862 | 1.00 | 61.55 | O |

| | | | | | | | | | | | |
|---|---|---|---|---|---|---|---|---|---|---|---|
| ANISOU | 2759 | O | HOH | 403 | 7951 | 7653 | 7782 | −64 | 16 | −30 | O |

| | | | | | | | | | | |
|---|---|---|---|---|---|---|---|---|---|---|
| HETATM | 2760 | O | HOH | 413 | 41.302 | 18.266 | 82.469 | 1.00 | 51.62 | O |

| | | | | | | | | | | | |
|---|---|---|---|---|---|---|---|---|---|---|---|
| ANISOU | 2760 | O | HOH | 413 | 6539 | 6611 | 6463 | 67 | −113 | −36 | O |

| | | | | | | | | | | |
|---|---|---|---|---|---|---|---|---|---|---|
| HETATM | 2761 | O | HOH | 414 | 37.305 | 36.992 | 70.772 | 1.00 | 55.58 | O |

| | | | | | | | | | | | |
|---|---|---|---|---|---|---|---|---|---|---|---|
| ANISOU | 2761 | O | HOH | 414 | 7055 | 7074 | 6990 | −12 | 73 | 29 | O |

| | | | | | | | | | | |
|---|---|---|---|---|---|---|---|---|---|---|
| HETATM | 2762 | O | HOH | 416 | −0.907 | 21.900 | 85.346 | 1.00 | 41.86 | O |

| | | | | | | | | | | | |
|---|---|---|---|---|---|---|---|---|---|---|---|
| ANISOU | 2762 | O | HOH | 416 | 5163 | 5398 | 5342 | 49 | 72 | 5 | O |

| | | | | | | | | | | |
|---|---|---|---|---|---|---|---|---|---|---|
| HETATM | 2763 | O | HOH | 417 | 6.864 | 32.422 | 79.415 | 1.00 | 52.77 | O |

| | | | | | | | | | | | |
|---|---|---|---|---|---|---|---|---|---|---|---|
| ANISOU | 2763 | O | HOH | 417 | 6687 | 6550 | 6815 | 172 | −1 | 17 | O |

| | | | | | | | | | | |
|---|---|---|---|---|---|---|---|---|---|---|
| HETATM | 2764 | O | HOH | 418 | 7.481 | 39.091 | 75.571 | 1.00 | 59.45 | O |

| | | | | | | | | | | | |
|---|---|---|---|---|---|---|---|---|---|---|---|
| ANISOU | 2764 | O | HOH | 418 | 7482 | 7522 | 7584 | 0 | 0 | −13 | O |

| | | | | | | | | | | |
|---|---|---|---|---|---|---|---|---|---|---|
| HETATM | 2785 | O | HOH | 419 | 27.524 | 39.365 | 73.684 | 1.00 | 25.45 | O |

| | | | | | | | | | | | |
|---|---|---|---|---|---|---|---|---|---|---|---|
| ANISOU | 2765 | O | HOH | 419 | 2857 | 3298 | 3514 | −125 | 281 | 584 | O |

| | | | | | | | | | | |
|---|---|---|---|---|---|---|---|---|---|---|
| HETATM | 2766 | O | HOH | 420 | −0.025 | 28.677 | 90.592 | 1.00 | 65.13 | O |

| | | | | | | | | | | | |
|---|---|---|---|---|---|---|---|---|---|---|---|
| ANISOU | 2766 | O | HOH | 420 | 8228 | 8291 | 8227 | −27 | 90 | −41 | O |

| | | | | | | | | | | |
|---|---|---|---|---|---|---|---|---|---|---|
| HETATM | 2767 | O | HOH | 425 | 40.888 | 16.331 | 73.180 | 1.00 | 61.78 | O |

| | | | | | | | | | | | |
|---|---|---|---|---|---|---|---|---|---|---|---|
| ANISOU | 2767 | O | HOH | 425 | 7832 | 7717 | 7926 | 9 | −4 | 26 | O |

| | | | | | | | | | | |
|---|---|---|---|---|---|---|---|---|---|---|
| HETATM | 2768 | O | HOH | 426 | 28.444 | 41.518 | 73.525 | 1.00 | 37.04 | O |

| | | | | | | | | | | | |
|---|---|---|---|---|---|---|---|---|---|---|---|
| ANISOU | 2768 | O | HOH | 426 | 4734 | 4158 | 5182 | −53 | 20 | −25 | O |

| | | | | | | | | | | |
|---|---|---|---|---|---|---|---|---|---|---|
| HETATM | 2769 | O | HOH | 427 | 16.016 | 3.824 | 82.316 | 1.00 | 57.82 | O |

| | | | | | | | | | | | |
|---|---|---|---|---|---|---|---|---|---|---|---|
| ANISOU | 2769 | O | HOH | 427 | 7247 | 7319 | 7403 | −26 | −41 | 16 | O |

| | | | | | | | | | | |
|---|---|---|---|---|---|---|---|---|---|---|
| HETATM | 2770 | O | HOH | 428 | 23.420 | 41.821 | 73.723 | 1.00 | 45.39 | O |

| | | | | | | | | | | | |
|---|---|---|---|---|---|---|---|---|---|---|---|
| ANISOU | 2770 | O | HOH | 428 | 5489 | 5889 | 5866 | −26 | −7 | 109 | O |

| | | | | | | | | | | |
|---|---|---|---|---|---|---|---|---|---|---|
| HETATM | 2771 | O | HOH | 429 | 12.812 | 4.190 | 66.701 | 1.00 | 59.20 | O |

| | | | | | | | | | | | |
|---|---|---|---|---|---|---|---|---|---|---|---|
| ANISOU | 2771 | O | HOH | 429 | 7463 | 7442 | 7588 | −30 | 30 | 2 | O |

| | | | | | | | | | | |
|---|---|---|---|---|---|---|---|---|---|---|
| HETATM | 2772 | O | HOH | 430 | 6.360 | 35.019 | 76.713 | 1.00 | 59.09 | O |

| | | | | | | | | | | | |
|---|---|---|---|---|---|---|---|---|---|---|---|
| ANISOU | 2772 | O | HOH | 430 | 7361 | 7558 | 7532 | −11 | 1 | −48 | O |

| | | | | | | | | | | |
|---|---|---|---|---|---|---|---|---|---|---|
| HETATM | 2773 | O | HOH | 432 | 4.119 | 32.067 | 92.209 | 1.00 | 51.99 | O |

| | | | | | | | | | | | |
|---|---|---|---|---|---|---|---|---|---|---|---|
| ANISOU | 2773 | O | HOH | 432 | 6557 | 6663 | 6534 | 4 | −80 | 80 | O |

| | | | | | | | | | | |
|---|---|---|---|---|---|---|---|---|---|---|
| HETATM | 2774 | O | HOH | 437 | 6.623 | 12.969 | 48.482 | 1.00 | 56.87 | O |

| | | | | | | | | | | | |
|---|---|---|---|---|---|---|---|---|---|---|---|
| ANISOU | 2774 | O | HOH | 437 | 7364 | 7116 | 7127 | 44 | −1 | −48 | O |

| | | | | | | | | | | |
|---|---|---|---|---|---|---|---|---|---|---|
| HETATM | 2775 | O | HOH | 440 | 12.750 | 40.608 | 70.163 | 1.00 | 55.18 | O |

| | | | | | | | | | | | |
|---|---|---|---|---|---|---|---|---|---|---|---|
| ANISOU | 2775 | O | HOH | 440 | 6954 | 6998 | 7015 | −28 | 47 | 23 | O |

| | | | | | | | | | | |
|---|---|---|---|---|---|---|---|---|---|---|
| HETATM | 2776 | O | HOH | 441 | 7.126 | 2.530 | 67.794 | 1.00 | 59.18 | O |

| | | | | | | | | | | | |
|---|---|---|---|---|---|---|---|---|---|---|---|
| ANISOU | 2776 | O | HOH | 441 | 7546 | 7460 | 7480 | −61 | −36 | −48 | O |

| | | | | | | | | | | |
|---|---|---|---|---|---|---|---|---|---|---|
| HETATM | 2777 | O | HOH | 443 | 46.670 | 25.492 | 68.517 | 1.00 | 61.08 | O |

| | | | | | | | | | | | |
|---|---|---|---|---|---|---|---|---|---|---|---|
| ANISOU | 2777 | O | HOH | 443 | 7683 | 7754 | 7769 | 27 | 72 | 13 | O |

| | | | | | | | | | | |
|---|---|---|---|---|---|---|---|---|---|---|
| HETATM | 2778 | O | HOH | 445 | 6.871 | 30.879 | 95.965 | 1.00 | 55.71 | O |

| | | | | | | | | | | | |
|---|---|---|---|---|---|---|---|---|---|---|---|
| ANISOU | 2778 | O | HOH | 445 | 7018 | 7146 | 7002 | 12 | −10 | 46 | O |

| | | | | | | | | | | |
|---|---|---|---|---|---|---|---|---|---|---|
| HETATM | 2779 | O | HOH | 451 | 15.622 | 12.679 | 58.773 | 1.00 | 61.40 | O |

| | | | | | | | | | | | |
|---|---|---|---|---|---|---|---|---|---|---|---|
| ANISOU | 2779 | O | HOH | 451 | 7791 | 7823 | 7716 | 9 | −88 | −11 | O |

| | | | | | | | | | | |
|---|---|---|---|---|---|---|---|---|---|---|
| HETATM | 2780 | O | HOH | 452 | 25.689 | 28.519 | 54.413 | 1.00 | 62.21 | O |

| | | | | | | | | | | | |
|---|---|---|---|---|---|---|---|---|---|---|---|
| ANISOU | 2780 | O | HOH | 452 | 7785 | 7966 | 7887 | −41 | 14 | 26 | O |

| | | | | | | | | | | |
|---|---|---|---|---|---|---|---|---|---|---|
| HETATM | 2781 | O | HOH | 457 | 41.314 | 16.986 | 88.613 | 1.00 | 48.52 | O |

| | | | | | | | | | | | |
|---|---|---|---|---|---|---|---|---|---|---|---|
| ANISOU | 2781 | O | HOH | 457 | 6011 | 6194 | 6231 | 92 | 5 | 47 | O |

| | | | | | | | | | | |
|---|---|---|---|---|---|---|---|---|---|---|
| HETATM | 2782 | O | HOH | 465 | 24.829 | 40.922 | 69.886 | 1.00 | 44.56 | O |

| | | | | | | | | | | | |
|---|---|---|---|---|---|---|---|---|---|---|---|
| ANISOU | 2782 | O | HOH | 465 | 5771 | 5485 | 5673 | 12 | 15 | 34 | O |

| | | | | | | | | | | |
|---|---|---|---|---|---|---|---|---|---|---|
| HETATM | 2783 | O | HOH | 467 | 6.762 | 36.046 | 61.809 | 1.00 | 56.72 | O |

| | | | | | | | | | | | |
|---|---|---|---|---|---|---|---|---|---|---|---|
| ANISOU | 2783 | O | HOH | 467 | 7186 | 7159 | 7205 | 4 | 3 | 8 | O |

| | | | | | | | | | | |
|---|---|---|---|---|---|---|---|---|---|---|
| HETATM | 2784 | O | HOH | 472 | 11.158 | 39.890 | 87.944 | 1.00 | 57.13 | O |

| | | | | | | | | | | | |
|---|---|---|---|---|---|---|---|---|---|---|---|
| ANISOU | 2784 | O | HOH | 472 | 7284 | 7170 | 7253 | −3 | 28 | 31 | O |

| | | | | | | | | | | |
|---|---|---|---|---|---|---|---|---|---|---|
| HETATM | 2785 | O | HOH | 473 | 31.538 | 12.268 | 75.175 | 1.00 | 51.92 | O |

| | | | | | | | | | | | |
|---|---|---|---|---|---|---|---|---|---|---|---|
| ANISOU | 2785 | O | HOH | 473 | 6705 | 6525 | 6496 | −25 | 51 | −14 | O |

| | | | | | | | | | | |
|---|---|---|---|---|---|---|---|---|---|---|
| HETATM | 2786 | O | HOH | 476 | 26.979 | 13.822 | 60.648 | 1.00 | 42.36 | O |

| | | | | | | | | | | | |
|---|---|---|---|---|---|---|---|---|---|---|---|
| ANISOU | 2786 | O | HOH | 476 | 5511 | 5199 | 5384 | 9 | 105 | 7 | O |

| | | | | | | | | | | |
|---|---|---|---|---|---|---|---|---|---|---|
| HETATM | 2787 | O | HOH | 477 | 21.273 | 37.456 | 63.586 | 1.00 | 54.55 | O |

| | | | | | | | | | | | |
|---|---|---|---|---|---|---|---|---|---|---|---|
| ANISOU | 2787 | O | HOH | 477 | 6835 | 6933 | 6957 | −34 | 40 | 48 | O |

| | | | | | | | | | | |
|---|---|---|---|---|---|---|---|---|---|---|
| HETATM | 2788 | O | HOH | 481 | 20.412 | 35.837 | 50.187 | 1.00 | 51.97 | O |

| | | | | | | | | | | | |
|---|---|---|---|---|---|---|---|---|---|---|---|
| ANISOU | 2788 | O | HOH | 481 | 6452 | 6741 | 6555 | −93 | 48 | 2 | O |

| | | | | | | | | | | |
|---|---|---|---|---|---|---|---|---|---|---|
| HETATM | 2789 | O | HOH | 482 | 39.521 | 42.897 | 65.639 | 1.00 | 58.39 | O |

| | | | | | | | | | | | |
|---|---|---|---|---|---|---|---|---|---|---|---|
| ANISOU | 2789 | O | HOH | 482 | 7504 | 7396 | 7285 | −33 | 69 | −7 | O |

| | | | | | | | | | | |
|---|---|---|---|---|---|---|---|---|---|---|
| HETATM | 2790 | O | HOH | 483 | 24.823 | 8.540 | 69.442 | 1.00 | 59.90 | O |

| | | | | | | | | | | | |
|---|---|---|---|---|---|---|---|---|---|---|---|
| ANISOU | 2790 | O | HOH | 483 | 7700 | 7472 | 7588 | −65 | −2 | 3 | O |

| | | | | | | | | | | |
|---|---|---|---|---|---|---|---|---|---|---|
| HETATM | 2791 | O | HOH | 487 | 5.944 | 34.999 | 92.476 | 1.00 | 56.11 | O |

| | | | | | | | | | | | |
|---|---|---|---|---|---|---|---|---|---|---|---|
| ANISOU | 2791 | O | HOH | 487 | 7054 | 7203 | 7062 | −16 | 42 | −19 | O |

| | | | | | | | | | | |
|---|---|---|---|---|---|---|---|---|---|---|
| HETATM | 2792 | O | HOH | 488 | 37.642 | 14.987 | 63.395 | 1.00 | 47.50 | O |

| | | | | | | | | | | | |
|---|---|---|---|---|---|---|---|---|---|---|---|
| ANISOU | 2792 | O | HOH | 488 | 5941 | 5990 | 6117 | 98 | −102 | 11 | O |

| | | | | | | | | | | |
|---|---|---|---|---|---|---|---|---|---|---|
| HETATM | 2793 | O | HOH | 489 | 41.015 | 38.881 | 64.470 | 1.00 | 55.54 | O |

| | | | | | | | | | | | |
|---|---|---|---|---|---|---|---|---|---|---|---|
| ANISOU | 2793 | O | HOH | 489 | 7016 | 6923 | 7165 | −8 | 23 | −31 | O |

| | | | | | | | | | | |
|---|---|---|---|---|---|---|---|---|---|---|
| HETATM | 2794 | O | HOH | 491 | 32.734 | 18.593 | 58.165 | 1.00 | 53.69 | O |

| | | | | | | | | | | | |
|---|---|---|---|---|---|---|---|---|---|---|---|
| ANISOU | 2794 | O | HOH | 491 | 6728 | 6899 | 6773 | 47 | −24 | −98 | O |

| | | | | | | | | | | |
|---|---|---|---|---|---|---|---|---|---|---|
| HETATM | 2795 | O | HOH | 493 | 8.740 | 7.839 | 89.511 | 1.00 | 58.03 | O |

| | | | | | | | | | | | |
|---|---|---|---|---|---|---|---|---|---|---|---|
| ANISOU | 2795 | O | HOH | 493 | 7350 | 7327 | 7372 | −11 | −45 | 70 | O |

| | | | | | | | | | | |
|---|---|---|---|---|---|---|---|---|---|---|
| HETATM | 2796 | O | HOH | 495 | 23.604 | 25.929 | 44.812 | 1.00 | 56.41 | O |

| | | | | | | | | | | | |
|---|---|---|---|---|---|---|---|---|---|---|---|
| ANISOU | 2796 | O | HOH | 495 | 7030 | 7253 | 7153 | 98 | 63 | −21 | O |

| | | | | | | | | | | |
|---|---|---|---|---|---|---|---|---|---|---|
| HETATM | 2797 | O | HOH | 500 | 0.474 | 27.705 | 86.600 | 1.00 | 48.77 | O |

| | | | | | | | | | | | |
|---|---|---|---|---|---|---|---|---|---|---|---|
| ANISOU | 2797 | O | HOH | 500 | 6246 | 6507 | 5777 | 331 | −175 | −101 | O |

| | | | | | | | | | | |
|---|---|---|---|---|---|---|---|---|---|---|
| HETATM | 2798 | O | HOH | 504 | 25.631 | 32.751 | 48.064 | 1.00 | 65.67 | O |

| | | | | | | | | | | | |
|---|---|---|---|---|---|---|---|---|---|---|---|
| ANISOU | 2798 | O | HOH | 504 | 8285 | 8326 | 8342 | −8 | 25 | 18 | O |

| | | | | | | | | | | |
|---|---|---|---|---|---|---|---|---|---|---|
| HETATM | 2799 | O | HOH | 506 | 19.513 | 4.664 | 81.284 | 1.00 | 62.16 | O |

| | | | | | | | | | | | |
|---|---|---|---|---|---|---|---|---|---|---|---|
| ANISOU | 2799 | O | HOH | 506 | 7807 | 7850 | 7961 | −7 | −67 | −14 | O |

| | | | | | | | | | | |
|---|---|---|---|---|---|---|---|---|---|---|
| HETATM | 2800 | O | HOH | 509 | 21.454 | 17.148 | 40.997 | 1.00 | 56.69 | O |

| | | | | | | | | | | | |
|---|---|---|---|---|---|---|---|---|---|---|---|
| ANISOU | 2800 | O | HOH | 509 | 6992 | 7285 | 7262 | 26 | −33 | 3 | O |

| | | | | | | | | | | |
|---|---|---|---|---|---|---|---|---|---|---|
| HETATM | 2801 | O | HOH | 514 | 18.388 | 40.807 | 69.422 | 1.00 | 59.00 | O |

| | | | | | | | | | | | |
|---|---|---|---|---|---|---|---|---|---|---|---|
| ANISOU | 2801 | O | HOH | 514 | 7435 | 7516 | 7467 | 9 | −10 | 19 | O |

| | | | | | | | | | | |
|---|---|---|---|---|---|---|---|---|---|---|
| HETATM | 2802 | O | HOH | 515 | 8.190 | 38.470 | 57.852 | 1.00 | 59.17 | O |

| | | | | | | | | | | | |
|---|---|---|---|---|---|---|---|---|---|---|---|
| ANISOU | 2802 | O | HOH | 515 | 7415 | 7446 | 7622 | −3 | −72 | 88 | O |

| | | | | | | | | | | |
|---|---|---|---|---|---|---|---|---|---|---|
| HETATM | 2803 | O | HOH | 517 | 43.777 | 32.298 | 64.059 | 1.00 | 61.31 | O |

| | | | | | | | | | | | |
|---|---|---|---|---|---|---|---|---|---|---|---|
| ANISOU | 2803 | O | HOH | 517 | 7705 | 7778 | 7813 | −33 | 72 | 75 | O |

| | | | | | | | | | | |
|---|---|---|---|---|---|---|---|---|---|---|
| HETATM | 2804 | O | HOH | 519 | 25.135 | 36.655 | 71.139 | 1.00 | 50.29 | O |

| | | | | | | | | | | | |
|---|---|---|---|---|---|---|---|---|---|---|---|
| ANISOU | 2804 | O | HOH | 519 | 6713 | 6286 | 6111 | 241 | −9 | 45 | O |

| | | | | | | | | | | |
|---|---|---|---|---|---|---|---|---|---|---|
| HETATM | 2805 | O | HOH | 522 | 16.923 | 7.483 | 41.655 | 1.00 | 52.27 | O |

| | | | | | | | | | | | |
|---|---|---|---|---|---|---|---|---|---|---|---|
| ANISOU | 2805 | O | HOH | 522 | 6598 | 6563 | 6700 | −41 | 48 | −17 | O |

| | | | | | | | | | | |
|---|---|---|---|---|---|---|---|---|---|---|
| HETATM | 2806 | O | HOH | 523 | 11.324 | 2.417 | 78.141 | 1.00 | 62.13 | O |

| | | | | | | | | | | | |
|---|---|---|---|---|---|---|---|---|---|---|---|
| ANISOU | 2806 | O | HOH | 523 | 7828 | 7925 | 7853 | 21 | 21 | 50 | O |

| | | | | | | | | | | |
|---|---|---|---|---|---|---|---|---|---|---|
| HETATM | 2807 | O | HOH | 526 | 33.616 | 13.704 | 92.622 | 1.00 | 52.56 | O |

| | | | | | | | | | | | |
|---|---|---|---|---|---|---|---|---|---|---|---|
| ANISOU | 2807 | O | HOH | 526 | 6856 | 6504 | 6609 | 2 | −19 | 32 | O |

| | | | | | | | | | | |
|---|---|---|---|---|---|---|---|---|---|---|
| HETATM | 2808 | O | HOH | 530 | 40.594 | 19.720 | 85.115 | 1.00 | 59.54 | O |

| | | | | | | | | | | | |
|---|---|---|---|---|---|---|---|---|---|---|---|
| ANISOU | 2808 | O | HOH | 530 | 7343 | 7643 | 7637 | 6 | −50 | 2 | O |

| | | | | | | | | | | |
|---|---|---|---|---|---|---|---|---|---|---|
| HETATM | 2809 | O | HOH | 532 | 15.076 | 31.487 | 94.187 | 1.00 | 54.80 | O |

| | | | | | | | | | | | |
|---|---|---|---|---|---|---|---|---|---|---|---|
| ANISOU | 2809 | O | HOH | 532 | 6794 | 7049 | 6979 | −22 | 16 | 3 | O |

| | | | | | | | | | | |
|---|---|---|---|---|---|---|---|---|---|---|
| HETATM | 2810 | O | HOH | 533 | 29.531 | 25.159 | 95.169 | 1.00 | 60.30 | O |

| | | | | | | | | | | | |
|---|---|---|---|---|---|---|---|---|---|---|---|
| ANISOU | 2810 | O | HOH | 533 | 7650 | 7631 | 7631 | −35 | −35 | 34 | O |

| | | | | | | | | | | |
|---|---|---|---|---|---|---|---|---|---|---|
| HETATM | 2811 | O | HOH | 537 | 28.865 | 20.427 | 53.190 | 1.00 | 61.62 | O |

| | | | | | | | | | | | |
|---|---|---|---|---|---|---|---|---|---|---|---|
| ANISOU | 2811 | O | HOH | 537 | 7694 | 7884 | 7833 | 7 | 63 | 7 | O |

| | | | | | | | | | | |
|---|---|---|---|---|---|---|---|---|---|---|
| HETATM | 2812 | O | HOH | 540 | 3.488 | 19.765 | 56.474 | 1.00 | 55.51 | O |

| | | | | | | | | | | | |
|---|---|---|---|---|---|---|---|---|---|---|---|
| ANISOU | 2812 | O | HOH | 540 | 6849 | 7169 | 7075 | 77 | 1 | 13 | O |

| | | | | | | | | | | |
|---|---|---|---|---|---|---|---|---|---|---|
| HETATM | 2813 | O | HOH | 544 | 1.976 | 26.521 | 93.596 | 1.00 | 55.30 | O |

| | | | | | | | | | | | |
|---|---|---|---|---|---|---|---|---|---|---|---|
| ANISOU | 2813 | O | HOH | 544 | 6950 | 7025 | 7035 | 50 | 74 | 42 | O |

| | | | | | | | | | | |
|---|---|---|---|---|---|---|---|---|---|---|
| HETATM | 2814 | O | HOH | 545 | 6.232 | 26.717 | 42.963 | 1.00 | 61.67 | O |

| | | | | | | | | | | | |
|---|---|---|---|---|---|---|---|---|---|---|---|
| ANISOU | 2814 | O | HOH | 545 | 7843 | 7854 | 7736 | 41 | −36 | −18 | O |

| | | | | | | | | | | |
|---|---|---|---|---|---|---|---|---|---|---|
| HETATM | 2815 | O | HOH | 547 | 15.978 | 43.004 | 78.376 | 1.00 | 54.93 | O |

| | | | | | | | | | | | |
|---|---|---|---|---|---|---|---|---|---|---|---|
| ANISOU | 2815 | O | HOH | 547 | 6775 | 6976 | 7120 | 29 | −47 | −37 | O |

| | | | | | | | | | | |
|---|---|---|---|---|---|---|---|---|---|---|
| HETATM | 2816 | O | HOH | 552 | 23.478 | 11.461 | 69.512 | 1.00 | 48.39 | O |

| | | | | | | | | | | | |
|---|---|---|---|---|---|---|---|---|---|---|---|
| ANISOU | 2816 | O | HOH | 552 | 6368 | 5943 | 6074 | 34 | 34 | −38 | O |

| | | | | | | | | | | |
|---|---|---|---|---|---|---|---|---|---|---|
| HETATM | 2817 | O | HOH | 564 | 31.768 | 13.802 | 72.785 | 1.00 | 50.67 | O |

| | | | | | | | | | | | |
|---|---|---|---|---|---|---|---|---|---|---|---|
| ANISOU | 2817 | O | HOH | 564 | 6430 | 6380 | 6444 | 37 | −36 | −82 | O |

| | | | | | | | | | | |
|---|---|---|---|---|---|---|---|---|---|---|
| HETATM | 2818 | O | HOH | 565 | 43.097 | 17.088 | 70.385 | 1.00 | 48.92 | O |

| | | | | | | | | | | | |
|---|---|---|---|---|---|---|---|---|---|---|---|
| ANISOU | 2818 | O | HOH | 565 | 6012 | 6253 | 6323 | 209 | 41 | −81 | O |

| | | | | | | | | | | |
|---|---|---|---|---|---|---|---|---|---|---|
| HETATM | 2819 | O | HOH | 578 | 25.824 | 12.068 | 89.747 | 1.00 | 49.43 | O |

| | | | | | | | | | | | |
|---|---|---|---|---|---|---|---|---|---|---|---|
| ANISOU | 2819 | O | HOH | 578 | 6241 | 6143 | 6397 | 115 | 23 | 18 | O |

| | | | | | | | | | | |
|---|---|---|---|---|---|---|---|---|---|---|
| HETATM | 2820 | O | HOH | 579 | 13.391 | 34.227 | 97.272 | 1.00 | 63.68 | O |

| | | | | | | | | | | | |
|---|---|---|---|---|---|---|---|---|---|---|---|
| ANISOU | 2820 | O | HOH | 579 | 8088 | 8000 | 8106 | −32 | 0 | −28 | O |

| | | | | | | | | | | |
|---|---|---|---|---|---|---|---|---|---|---|
| HETATM | 2821 | O | HOH | 583 | 16.532 | 18.164 | 87.980 | 1.00 | 51.16 | O |

| | | | | | | | | | | | |
|---|---|---|---|---|---|---|---|---|---|---|---|
| ANISOU | 2821 | O | HOH | 583 | 6539 | 6406 | 6493 | −38 | 73 | 105 | O |

| | | | | | | | | | | |
|---|---|---|---|---|---|---|---|---|---|---|
| HETATM | 2822 | O | HOH | 591 | 24.443 | 15.964 | 52.058 | 1.00 | 50.29 | O |

| | | | | | | | | | | | |
|---|---|---|---|---|---|---|---|---|---|---|---|
| ANISOU | 2822 | O | HOH | 591 | 6313 | 6394 | 6401 | −2 | 10 | 8 | O |

| | | | | | | | | | | |
|---|---|---|---|---|---|---|---|---|---|---|
| HETATM | 2823 | O | HOH | 592 | 11.355 | 36.376 | 65.514 | 1.00 | 52.43 | O |

| | | | | | | | | | | | |
|---|---|---|---|---|---|---|---|---|---|---|---|
| ANISOU | 2823 | O | HOH | 592 | 6660 | 6474 | 6786 | 67 | −21 | 4 | O |

| | | | | | | | | | | |
|---|---|---|---|---|---|---|---|---|---|---|
| HETATM | 2824 | O | HOH | 593 | 37.256 | 22.800 | 90.212 | 1.00 | 43.47 | O |

| | | | | | | | | | | | |
|---|---|---|---|---|---|---|---|---|---|---|---|
| ANISOU | 2824 | O | HOH | 593 | 5247 | 5701 | 5566 | −27 | −83 | 130 | O |

| | | | | | | | | | | |
|---|---|---|---|---|---|---|---|---|---|---|
| HETATM | 2825 | O | HOH | 600 | 18.701 | 9.992 | 44.274 | 1.00 | 55.16 | O |

| | | | | | | | | | | | |
|---|---|---|---|---|---|---|---|---|---|---|---|
| ANISOU | 2825 | O | HOH | 600 | 6999 | 7033 | 6926 | 47 | 14 | −113 | O |

| | | | | | | | | | | |
|---|---|---|---|---|---|---|---|---|---|---|
| HETATM | 2826 | O | HOH | 615 | 25.210 | 19.141 | 47.805 | 1.00 | 57.78 | O |

| | | | | | | | | | | | |
|---|---|---|---|---|---|---|---|---|---|---|---|
| ANISOU | 2826 | O | HOH | 615 | 7249 | 7324 | 7380 | 79 | 76 | 19 | O |

| | | | | | | | | | | |
|---|---|---|---|---|---|---|---|---|---|---|
| HETATM | 2827 | O | HOH | 616 | 22.084 | 11.729 | 51.994 | 1.00 | 58.99 | O |

| | | | | | | | | | | | |
|---|---|---|---|---|---|---|---|---|---|---|---|
| ANISOU | 2827 | O | HOH | 616 | 7480 | 7441 | 7493 | 60 | 2 | −88 | O |

What is claimed is:

1. A composition comprising a polypeptide comprising a SUMO protease catalytic domain in a trapped proteolytic deacylation intermediate complex with its substrate.

2. The composition of claim 1, wherein the complexed molecules have a crystalline structure.

3. The composition of claim 1, wherein the SUMO protease is Ulp1.

4. The composition of claim 3, wherein the substrate is Smt3.

5. The composition of claim 3, wherein the polypeptide consists of the catalytic domain of Ulp1.

6. The composition of claim 2, wherein the complex has crystal structure coordinates as shown in Table 1.

7. A method of forming a complex of a polypeptide comprising a catalytic domain of a protease with its substrate, which method comprises:
(a) combining the protease with its substrate in a molar ratio to produce a mixture;
(b) adding a reducing agent capable of trapping a proteolytic deacylation intermediate complex of the protease and the substrate in amount effective to trap an isolatable amount of the complex; and
(c) adjusting the pH of the mixture to about 7.0.

8. The method of claim 7, wherein steps (a) and (b) are performed simultaneously.

9. The method of claim 7, wherein the pH is adjusted to about 7.0 by dialyzing the mixture.

10. The method of claim 9, further comprising isolating the complex after dialysis.

11. The method of claim 9, wherein the protease is a SUMO protease.

12. The method of claim 7 where the protease is a polypeptide comprising a catalytic domain of Ulp1.

13. The method of claim 12 where the substrate is Smt3.

14. The method of claim 13 where Ulp1 and Smt3 are present in a molar ratio from 1:1 to 1:5.

15. The method of claim 14 where Ulp1 and Smt3 are present in a molar ratio of 1:3.

16. The method of claim 7, wherein the reducing agent is sodium borohydride.

17. A polynucleotide comprising a sequence, wherein the sequence encodes a mutant Ulp1.

18. The polynucleotide of claim 17, wherein the mutant Ulp1 comprises the amino acid sequence shown in SEQ ID NO:2, but contains an amino acid substitution at a position selected from the group consisting of 432, 448, 451, 455, 472, 474, 489, 490, 493, and 515.

19. A recombinant vector comprising the polynucleotide sequence of claim 17.

20. The recombinant vector of claim 19, where the polynucleotide encodes a mutant Ulp1 comprising an amino acid substitution at position 432, 448, 451, 455, 472, 474, 489, 490, 493, and/or 515.

21. A host cell comprising the recombinant vector of claim 19.

22. A host cell comprising the recombinant vector of claim 20.

23. A process of producing mutant Ulp1, comprising culturing the host cell of claim 21 under conditions whereby the polynucleotide encoding the mutant Ulp1 is expressed.

24. A mutant Ulp1 polypeptide.

25. The polypeptide of claim 24, wherein the mutant Ulp1 comprising an amino acid substitution at position 432, 448, 451, 455, 472, 474, 489, 490, 493, and/or 515 of SEQ ID NO: 2.

26. A method of identifying potential substrates of a cysteine protease by rational drug design, which method comprises designing candidate substrates that would form interactions with catalytic site amino acids identified from computer modeling based on the crystal structure of the composition of claim 2.

27. The method according to claim 26, wherein the crystal structure has a catalytic site having crystal structure coordinates as shown in Table 1.

28. The method according to claim 26, which further comprises synthesizing the candidate substrate and determining whether the candidate substrate interacts with the cysteine protease.

29. The method according to claim 26, wherein the candidate substrate is a cysteine protease inhibitor.

30. The method of claim 26, wherein the protease is Ulp1.


SEQUENCE LISTING (2 sequences)

SEQ ID NO: 1 - DNA, 1866 bp, Saccharomyces cerevisiae
atgtcagttgaagtagataagcaccggaacacactacagtatcataaaaagaacccttactcccctttattctccccaatttctacatataggtgttatcctcgggtattgaacaatccctccgagtctagaagatcagccagttttagtggtatttataaaaaaagaaccaatacgtcaagattcaattatttaaacgaccgccgtgttttatcaatggaagaatcaatgaaagatgggtcagatagagctagtaaagctggttttataggaggcataagagaaactctttggaactcaggtaagtacttatggcacacatttgtgaaaaacgaacctcgcaattttgatggttctgaagtagaagcaagtggtaacagcgacgttgagagcagaagttctggaagtaggagcagtgacgtaccatatggtctacgtgaaaattattcctcggatacaagaaaacacaaattcgatacgtcgacgtgggccttaccaaataaaaggagaagaatcgaaagtgaaggtgtggggacaccttcaacctcaccaatcagctctttggcttctcaaaaaagcaattgtgatagtgacaatagcataactttttcgagagatccttttggttggaataagtggaaaacaagtgctattggttctaactcagaaaataacacttctgatcagaaaaatagctacgacaggcgacagtatgggacagcctttattagaaagaaaaaagttgcaaaacagaacattaacaatactaaactggtgtcgagagcacaatctgaggaagtaacatatttacgacaaatattcaacggagaatataaagttccaaaaatattaaaagaagaaagagaaagacagttaaaattaatggatatggataaggaaaaagacactggcttaaaaaagtctataatcgacttaactgaaaagatcaaaacaattttaattgagaacaacaagaacagactacagacaagaaatgaaaatgatgatgatttagtatttgttaaagaaaaaaagatatcttctttggaaaggaaacataaggattacttaaatcaaaagttgaagtttgatagatctatattagagtttgagaaagacttcaaaagatataacgaaattttaaatgaaagaaagaagattcaagaagatcttaaaaaaaagaaagaacaattggccaagaagaaacttgttcctgaattaaatgaaaaagacgatgaccaagtacaaaaagctttggcatctagagaaaatactcagttaatgaatagagataatatagagataacagtacgtgattttaagaccttggcaccacgaagatggctaaatgacactatcattgagttttttatgaaatacattgaaaaatctacccctaatacagtggcgtttaattcatttttctataccaatttatcagaaaggggttatcaaggcgtccggaggtggatgaagagaaagaagacacaaattgataaacttgataaaatctttacaccaataaatttgaaccaatcccactgggcgttgggcataattgatttaaaaaagaaaactataggttacgtagattcattatcgaatggtccaaatgctatgagtttcgctatactgactgacttgcaaaaatatgttatggaggaaagtaagcatacaataggagaagactttgatttgattcatttagattgtccgcagcaaccaaatggctacgactgtggaatatatgtttgtatgaatactctctatggaagtgcagatgcgccattggattttgattataaagatgcgattaggatgagaagatttattgcccatttgattttaaccgacgctttaaaatag

SEQ ID NO: 2 - protein, 621 aa, Saccharomyces cerevisiae
MSVEVDKHRNTLQYHKKNPYSPLFSPISTYRCYPRVLNNPSESRRSASFSGIYKKRTNTSRFNYLNDRRVLSMEESMKDGSDRASKAGFIGGIRETLWNSGKYLWHTFVKNEPRNFDGSEVEASGNSDVESRSSGSRSSDVPYGLRENYSSDTRKHKFDTSTWALPNKRRRIESEGVGTPSTSPISSLASQKSNCDSDNSITFSRDPFGWNKWKTSAIGSNSENNTSDQKNSYDRRQYGTAFIRKKKVAKQNINNTKLVSRAQSEEVTYLRQIFNGEYKVPKILKEERERQLKLMDMDKEKDTGLKKSIIDLTEKIKTILIENNKNRLQTRNENDDDLVFVKEKKISSLERKHKDYLNQKLKFDRSILEFEKDFKRYNEILNERKKIQEDLKKKKEQLAKKKLVPELNEKDDDQVQKALASRENTQLMNRDNIEITVRDFKTLAPRRWLNDTIIEFFMKYIEKSTPNTVAFNSFFYTNLSERGYQGVRRWMKRKKTQIDKLDKIFTPINLNQSHWALGIIDLKKKTIGYVDSLSNGPNAMSFAILTDLQKYVMEESKHTIGEDFDLIHLDCPQQPNGYDCGIYVCMNTLYGSADAPLDFDYKDAIRMRRFIAHLILTDALK